(19) United States
(12) Patent Application Publication
(10) Pub. No.: US 2026/0015337 A1
(43) Pub. Date: Jan. 15, 2026

(54) COMPOUNDS FOR TREATMENT A CORONAVIRUS INFECTION

(71) Applicant: Trawsfynydd Therapeutics, Inc., San Diego, CA (US)

(72) Inventors: Boris Rogovoy, Needham, MA (US); Volodymyr Kysil, San Diego, CA (US); Vladimir Berishvili, Mölndal (SE); Charles David Pauza, Gaithersburg, MD (US); Juan Carlos Zapata, Ijamsville, MD (US); Sandra Margarita Medina Moreno, Rockville, MD (US); Haishan Li, North Potomac, MD (US); Andrew Orry, San Diego, CA (US); Polo Chun-Hung Lam, San Diego, CA (US); Ruben Abagyan, La Jolla, CA (US); Nikolay Savchuk, San Diego, CA (US)

(21) Appl. No.: 18/875,724

(22) PCT Filed: Jun. 16, 2023
(86) PCT No.: PCT/US2023/068578

§ 371 Date: Dec. 16, 2024

Related U.S. Application Data
(60) Provisional application No. 63/353,354, filed on Jun. 17, 2022.
(60) Provisional application No. 63/414,287, filed on Oct. 7, 2022.

(51) Int. Cl. C07D 401/12; A61K 31/436; A61K 31/4375; A61K 31/4439; A61K 31/4704; A61K 31/4709; A61K 31/498; A61K 31/506; C07D 215/227; C07D 401/14; C07D 403/12; C07D 405/14; C07D 413/14; C07D 417/14; C07D 471/04; C07D 491/048; C07D 491/052

(52) U.S. Cl. CPC C07D 401/12; A61K 31/436; A61K 31/4375; A61K 31/4439; A61K 31/4704; A61K 31/4709; A61K 31/498; A61K 31/506; C07D 215/227; C07D 401/14; C07D 403/12; C07D 405/14; C07D 413/14; C07D 417/14; C07D 471/04; C07D 491/048; C07D 491/052

(57) ABSTRACT

The present invention is generally directed to inhibitors of SARS-CoV-2-related 3C-like protease (Mpro) useful in the treatment of coronavirus infection and having the Formula (A):

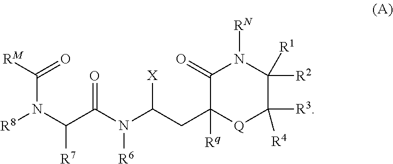

### CROSS-REFERENCE TO RELATED APPLICATIONS

This application claims priority to and benefit of U.S. Provisional Patent Application Ser. No. 63/353,354 filed Jun. 17, 2022, entitled “Compounds for treatment of a coronavirus infection”, and U.S. Provisional Patent Application Ser. No. 63/414,287 filed Oct. 7, 2022, entitled “Compounds for treatment of a coronavirus infection” the disclosure of which are incorporated by reference in its entirety for all purposes.

### FIELD OF INVENTION

The present invention is directed to inhibitors of coronavirus replication activity. The inhibitors described herein can be useful for the treatment of diseases caused by coronavirus infection including COVID-19 resulting from SARS-CoV-2 infection. In particular, the invention is concerned with compounds and pharmaceutical compositions that inhibit the SARS-CoV-2-related 3C-like protease, methods of treating coronavirus infection, and methods of synthesizing these compounds.

### BACKGROUND

Coronaviruses are a large family of viruses that usually cause mild to moderate upper-respiratory tract illnesses in humans. However, three coronaviruses have caused more serious and fatal diseases in people: SARS coronavirus (SARS-CoV), which emerged in November 2002 and causes severe acute respiratory syndrome (SARS); MERS coronavirus (MERS-CoV), which emerged in 2012 and causes Middle East respiratory syndrome (MERS); and SARS-CoV-2, which emerged in 2019 and causes coronavirus disease 2019 (COVID-19).

Infection with severe acute respiratory syndrome coronavirus 2, or SARS-CoV-2, causes coronavirus disease 2019 (COVID-19).

The virus that causes COVID-19 spreads easily among people. Data has shown that the COVID-19 virus spreads mainly from person to person among those in close contact (within about 6 feet, or 2 meters). The COVID-19 virus can spread from someone who is infected but has no symptoms. This is called asymptomatic transmission. The COVID-19 virus can also spread from someone who is infected but hasn't developed symptoms yet. This is called presymptomatic transmission. It's possible to contract COVID-19 upon reinfection with SARS-CoV-2 and this may happen more than once.

Whole genome sequencing showed that SARS-CoV-2 shares 79.6% of sequence identical to SARS-CoV. SARS-CoV-2 appears to have a relatively high transmission rate among humans and causes severe and fatal pneumonia and other damages, threatening people at all ages, especially seniors and persons with pre-existing conditions including immune deficiency, lung disease, metabolic disease and others. As the number of infections and deaths are increasing rapidly, there is an urgent call for drug development against COVID-19.

SARS-CoV-2 is a complex virus consisting of 3 open reading frames expressing polyproteins. The polyproteins under proteolytic processing into their mature, active forms. While the spike glycoprotein is subject to cleavage by hoist proteases Cathepsin L and TMPRSS2, the remaining proteolytic processing steps are mediated by one of two virally-encoded proteases designated Main protease (Mpro or 3C-like protease (3CLpro)) and the papain-like protease PLpro. Mpro plays a vital role in maturation of viral proteins require for RNA replication and is a preferred target for the development of direct acting antiviral drugs capable of inhibiting SARS-CoV-2 infection. The Mpro enzyme of SARS-CoV-2 is similar to SARS-CoV by about 96%.

In view of the ongoing SARS-CoV-2 spread that has caused the current worldwide COVID-19 outbreak, it is desirable to have new methods of inhibiting SARS-CoV-2 viral replication and of treating COVID-19 in patients.

### SUMMARY

The first aspect of the invention relates to compound of Formula (A):

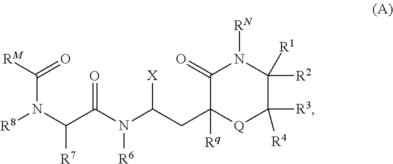

or a pharmaceutically acceptable salt, stereoisomer, solvate, prodrug, or tautomer thereof, wherein:
   each R^{N} is independently selected from H, C₁-C₆ alkyl, cycloalkyl;
   Q is CR⁵R^{Q} or NR^{Q};
   R^{q} is H;
   R^{Q} is R^{N}; or
   R^{q} and R^{Q} together form a bond;
   R^{M} is selected from

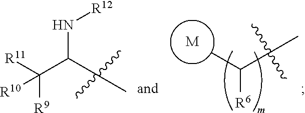

M is selected from 5-10 membered heterocyclyl or 5-10 membered heteroaryl, wherein the heterocyclyl or heteroaryl is optionally substituted with one or more R¹⁶;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R² together are oxo;
or R² and R³ together form a double bond;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
each R⁶ is independently selected from H, C₁-C₆ alkyl, cycloalkyl, —CH₂-aryl, wherein the alkyl, cycloalkyl, or aryl is optionally substituted with one or more R¹⁶;
R⁷ is selected from —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
or R⁷ and R⁸ together with the atoms to which they are attached and any intervening atoms, form 5-7 membered monocyclic or bicyclic heterocyclyl optionally substituted with one or more R³;
R⁹, R¹⁰, R¹¹ are each independently selected from C₁-C₆ alkyl, cycloalkyl, halogen, —CN;
R¹² is selected from R⁶—S(O)₂—, R⁶—C(O)—, R⁶—O—C(O)—, heteroaryl, wherein the heteroaryl is optionally substituted with one or more R¹⁶;
each R¹³ is independently selected from halogen, —OH, —CN, —C₁-C₆ alkyl, —C₁-C₆ halogenalkyl;
two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
R¹⁴ and R¹⁵ are each independently selected from H, C₁-C₆ alkyl, cycloalkyl;
or R¹⁴ and R¹⁵ together with the atoms to which they are attached and any intervening atoms, form heterocyclyl optionally substituted with one or more halogen, —OH, —CN, C₁-C₆ alkyl;
each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
n is an integer selected from 0, 1, 2;
m is an integer selected from 0 and 1;
provided that when R^{q} is H then R¹ and R⁴ together with the atoms to which they are attached and any intervening atoms, form a 3-10 membered cycloalkyl, a 6-10 membered aryl, a 3-14 membered heterocycle, or a 5-6 membered heteroaryl;
wherein,
   cycloalkyl is a mono or polycyclic saturated carbon rings containing 3-18 carbon atoms;
   aryl is a cyclic, aromatic hydrocarbon groups that have 1 to 3 aromatic rings;
   heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
   heteroaryl is a monovalent monocyclic or a polycyclic aromatic radical of 5 to 24 ring atoms, containing one or more ring heteroatoms selected from N, O, S, P, or B, the remaining ring atoms being C.

In another aspect the invention relates to compounds of Formula (I):

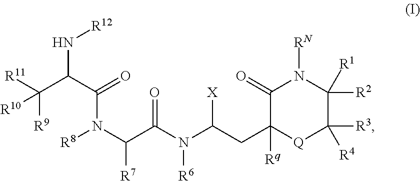

or a pharmaceutically acceptable salt, stereoisomer, solvate, prodrug, or tautomer thereof, wherein:
   each R^{N} is independently selected from H, C₁-C₆ alkyl, cycloalkyl;
   Q is CR⁵R^{Q} or NR^{Q};
   R^{q} is H;
   R^{Q} is R^{N}; or
   R^{q} and R^{Q} together form a bond;
   R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   or R¹ and R² together are oxo;
   or R² and R³ together form a double bond;
   R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
   R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   each R⁶ is independently selected from H, C₁-C₆ alkyl, cycloalkyl, —CH₂-aryl, wherein the alkyl, cycloalkyl, or aryl is optionally substituted with one or more R¹⁶;
   R⁷ is selected from —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
   R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
   or R⁷ and R⁸ together with the atoms to which they are attached and any intervening atoms, form 5-7 membered monocyclic or bicyclic heterocyclyl optionally substituted with one or more R¹³;
   R⁹, R¹⁰, R¹¹ are each independently selected from C₁-C₆ alkyl, cycloalkyl, halogen, —CN;
   R¹² is selected from R⁶—S(O)₂—, R⁶—C(O)—, R⁶—O—C(O)—, heteroaryl, wherein the heteroaryl is optionally substituted with one or more R¹⁶;
   each R¹³ is independently selected from halogen, —OH, —CN, —C₁-C₆ alkyl, —C₁-C₆ halogenalkyl;
   two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
   R¹⁴ and R¹⁵ are each independently selected from H, C₁-C₆ alkyl, cycloalkyl;
   or R¹⁴ and R¹⁵ together with the atoms to which they are attached and any intervening atoms, form heterocyclyl optionally substituted with one or more halogen, —OH, —CN, C₁-C₆ alkyl;
   each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
   two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
   X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
   n is an integer selected from 0, 1, 2;
   provided that when R^{q} is H then R¹ and R⁴ together with the atoms to which they are attached and any intervening atoms, form a 3-10 membered cycloalkyl, a 6-10 membered aryl, a 3-14 membered heterocycle, or a 5-6 membered heteroaryl;
wherein,
   cycloalkyl is a mono or polycyclic saturated carbon rings containing 3-18 carbon atoms;
   aryl is a cyclic, aromatic hydrocarbon groups that have 1 to 3 aromatic rings;
   heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
   heteroaryl is a monovalent monocyclic or a polycyclic aromatic radical of 5 to 24 ring atoms, containing one or more ring heteroatoms selected from N, O, S, P, or B, the remaining ring atoms being C.

In another aspect the invention relates to compounds of Formula (II):

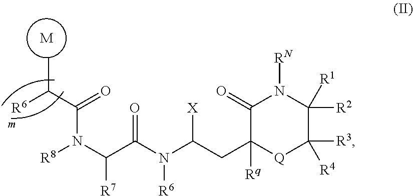

or a pharmaceutically acceptable salt, stereoisomer, solvate, prodrug, or tautomer thereof, wherein:
   each R^{N} is independently selected from H, C₁-C₆ alkyl, cycloalkyl;
   Q is CR⁵R^{Q} or NR^{Q};
   R^{q} is H;
   R^{Q} is R^{N}; or
   R^{q} and R^{Q} together form a bond;
   M is selected from 5-10 membered heterocyclyl or 5-10 membered heteroaryl, wherein the heterocyclyl or heteroaryl is optionally substituted with one or more R¹⁶;
   R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   or R¹ and R² together are oxo;
   or R² and R³ together form a double bond;
   R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
   R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O— cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   each R⁶ is independently selected from H, C₁-C₆ alkyl, cycloalkyl, —CH₂-aryl, wherein the alkyl, cycloalkyl, or aryl is optionally substituted with one or more R¹⁶;
   R⁷ is selected from —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
   R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
   or R⁷ and R⁸ together with the atoms to which they are attached and any intervening atoms, form 5-7 membered monocyclic or bicyclic heterocyclyl optionally substituted with one or more R¹³;
   each R¹³ is independently selected from halogen, —OH, —CN, —C₁-C₆ alkyl, —C₁-C₆ halogenalkyl;
   two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
   R¹⁴ and R¹⁵ are each independently selected from H, C₁-C₆ alkyl, cycloalkyl;
   or R¹⁴ and R¹⁵ together with the atoms to which they are attached and any intervening atoms, form heterocyclyl optionally substituted with one or more halogen, —OH, —CN, C₁-C₆ alkyl;
   each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
   two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
   X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
   m is an integer selected from 0 and 1;
   n is an integer selected from 0, 1, 2;
   provided that when R^{q} is H then R¹ and R⁴ together with the atoms to which they are attached and any intervening atoms, form a 3-10 membered cycloalkyl, a 6-10 membered aryl, a 3-14 membered heterocycle, or a 5-6 membered heteroaryl;
wherein,
   cycloalkyl is a mono or polycyclic saturated carbon rings containing 3-18 carbon atoms;
   aryl is a cyclic, aromatic hydrocarbon groups that have 1 to 3 aromatic rings;
   heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
   heteroaryl is a monovalent monocyclic or a polycyclic aromatic radical of 5 to 24 ring atoms, containing one or more ring heteroatoms selected from N, O, S, P, or B, the remaining ring atoms being C.

Another aspect of the invention is directed to pharmaceutical compositions comprising a compound of Formula (A), or a pharmaceutically acceptable salt, hydrate, solvate, prodrug, stereoisomer, or tautomer thereof and a pharmaceutically acceptable carrier.

The pharmaceutically acceptable carrier may further include an excipient, diluent, or surfactant.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier.

The pharmaceutically acceptable carrier may further include an excipient, diluent, or surfactant.

Another aspect of the invention is directed to pharmaceutical compositions comprising a compound of Formula (II), or a pharmaceutically acceptable salt, hydrate, solvate, prodrug, stereoisomer, or tautomer thereof and a pharmaceutically acceptable carrier.

The pharmaceutically acceptable carrier may further include an excipient, diluent, or surfactant.

Another aspect of the invention relates to a method of treating coronavirus infection such as COVID-19. The method comprises administering to a patient in need of a treatment coronavirus infection such as COVID-19 an effective amount of a compound of Formula (A) or a pharmaceutically acceptable salt, hydrate, solvate, prodrug, stereoisomer, tautomer, or pharmaceutical composition thereof.

Another aspect of the invention relates to a method of treating coronavirus infection such as COVID-19. The method comprises administering to a patient in need of a treatment coronavirus infection such as COVID-19 an effective amount of a compound of Formula (I) or a pharmaceutically acceptable salt, hydrate, solvate, prodrug, stereoisomer, tautomer, or pharmaceutical composition thereof.

Another aspect of the invention relates to a method of treating coronavirus infection such as COVID-19. The method comprises administering to a patient in need of a treatment coronavirus infection such as COVID-19 an effective amount of a compound of Formula (II) or a pharmaceutically acceptable salt, hydrate, solvate, prodrug, stereoisomer, tautomer, or pharmaceutical composition thereof.

Another aspect of the invention is directed to a method of inhibiting or preventing SARS-CoV-2 viral replication. The method involves administering to a patient in need thereof an effective amount of a compound of Formula (A) or a pharmaceutically acceptable salt, hydrate, solvate, prodrug, stereoisomer, tautomer, or pharmaceutical composition thereof.

Another aspect of the invention is directed to a method of inhibiting or preventing SARS-CoV-2 viral replication. The method involves administering to a patient in need thereof an effective amount of a compound of Formula (I) or a pharmaceutically acceptable salt, hydrate, solvate, prodrug, stereoisomer, tautomer, or pharmaceutical composition thereof.

Another aspect of the invention is directed to a method of inhibiting or preventing SARS-CoV-2 viral replication. The method involves administering to a patient in need thereof an effective amount of a compound of Formula (II) or a pharmaceutically acceptable salt, hydrate, solvate, prodrug, stereoisomer, tautomer, or pharmaceutical composition thereof.

Another aspect of the present invention relates to compounds of Formula (A) or pharmaceutically acceptable salts, hydrates, solvates, prodrugs, stereoisomers, tautomers, or pharmaceutical compositions thereof, for use in the manufacture of a medicament for inhibiting or preventing SARS-CoV-2 viral replication.

Another aspect of the present invention relates to compounds of Formula (I) or pharmaceutically acceptable salts, hydrates, solvates, prodrugs, stereoisomers, tautomers, or pharmaceutical compositions thereof, for use in the manufacture of a medicament for inhibiting or preventing SARS-CoV-2 viral replication.

Another aspect of the present invention relates to compounds of Formula (II) or pharmaceutically acceptable salts, hydrates, solvates, prodrugs, stereoisomers, tautomers, or pharmaceutical compositions thereof, for use in the manufacture of a medicament for inhibiting or preventing SARS-CoV-2 viral replication.

Another aspect of the present invention relates to the use of compounds of Formula (A) or pharmaceutically acceptable salts, hydrates, solvates, prodrugs, stereoisomers, tautomers, or pharmaceutical compositions thereof, in the treatment of a disease or disorder associated with SARS-CoV-2 viral infection.

Another aspect of the present invention relates to the use of compounds of Formula (I) or pharmaceutically acceptable salts, hydrates, solvates, prodrugs, stereoisomers, tautomers, or pharmaceutical compositions thereof, in the treatment of a disease or disorder associated with SARS-CoV-2 viral infection.

Another aspect of the present invention relates to the use of compounds of Formula (II) or pharmaceutically acceptable salts, hydrates, solvates, prodrugs, stereoisomers, tautomers, or pharmaceutical compositions thereof, in the treatment of a disease or disorder associated with SARS-CoV-2 viral infection.

Another aspect of the present invention relates to compounds of Formula (A) or pharmaceutically acceptable salts, hydrates, solvates, prodrugs, stereoisomers, tautomers, or pharmaceutical compositions thereof, for use in the manufacture of a medicament for treating or preventing a viral infection disclosed herein.

Another aspect of the present invention relates to compounds of Formula (I) or pharmaceutically acceptable salts, hydrates, solvates, prodrugs, stereoisomers, tautomers, or pharmaceutical compositions thereof, for use in the manufacture of a medicament for treating or preventing a viral infection disclosed herein.

Another aspect of the present invention relates to compounds of Formula (II) or pharmaceutically acceptable salts, hydrates, solvates, prodrugs, stereoisomers, tautomers, or pharmaceutical compositions thereof, for use in the manufacture of a medicament for treating or preventing a viral infection disclosed herein.

Another aspect of the invention is directed to a method of treating or preventing a coronavirus infection such as COVID-19 in a subject in need thereof. The method involves administering to a patient in need of the treatment an effective amount of a compound of Formula (A) or a pharmaceutically acceptable salt, hydrate, solvate, prodrug, stereoisomer, tautomer, or pharmaceutical composition thereof.

Another aspect of the invention is directed to a method of treating or preventing a coronavirus infection such as COVID-19 in a subject in need thereof. The method involves administering to a patient in need of the treatment an effective amount of a compound of Formula (I) or a pharmaceutically acceptable salt, hydrate, solvate, prodrug, stereoisomer, tautomer, or pharmaceutical composition thereof.

Another aspect of the invention is directed to a method of treating or preventing a coronavirus infection such as COVID-19 in a subject in need thereof. The method involves administering to a patient in need of the treatment an effective amount of a compound of Formula (II) or a pharmaceutically acceptable salt, hydrate, solvate, prodrug, stereoisomer, tautomer, or pharmaceutical composition thereof.

Another aspect of the present invention relates to the use of compounds of Formula (A), or pharmaceutically acceptable salts, hydrates, solvates, prodrugs, stereoisomers, tautomers, or pharmaceutical compositions thereof, in the treatment of a coronavirus infection.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a coronavirus infection.

Another aspect of the present invention relates to the use of compounds of Formula (II), or pharmaceutically acceptable salts, hydrates, solvates, prodrugs, stereoisomers, tautomers, or pharmaceutical compositions thereof, in the treatment of a coronavirus infection.

The present invention further provides methods of treating a coronavirus infection, comprising administering to a patient suffering from a coronavirus infection a compound of Formula (A), or a pharmaceutically acceptable salt, hydrate, solvate, prodrug, stereoisomer, tautomer, or pharmaceutical composition thereof.

The present invention further provides methods of treating a coronavirus infection, comprising administering to a patient suffering from a coronavirus infection a compound of Formula (I), or a pharmaceutically acceptable salt, hydrate, solvate, prodrug, stereoisomer, tautomer, or pharmaceutical composition thereof.

The present invention further provides methods of treating a coronavirus infection, comprising administering to a patient suffering from a coronavirus infection a compound of Formula (II), or a pharmaceutically acceptable salt, hydrate, solvate, prodrug, stereoisomer, tautomer, or pharmaceutical composition thereof.

The present invention provides inhibitors of viral replication activity that are therapeutic agents in the treatment of a coronavirus infection.

The present invention provides SARS-CoV-2-related 3C-like protease inhibitors that are therapeutic agents in the treatment of a coronavirus infection.

The present invention provides SARS-CoV-2-related Mpro protease inhibitors that are therapeutic agents in the treatment of a coronavirus infection.

The present invention further provides compounds and compositions with an improved efficacy and safety profile relative to known inhibitors SARS-CoV-2-related 3C-like protease.

The present invention further provides methods of treating a disease or disorder associated with coronavirus infection, comprising administering to a patient suffering from coronavirus infection a compound of Formula (A) or a pharmaceutically acceptable salt, hydrate, solvate, prodrug, stereoisomer, tautomer, or pharmaceutical composition thereof.

The present invention further provides methods of treating a disease or disorder associated with coronavirus infection, comprising administering to a patient suffering from coronavirus infection a compound of Formula (I) or a pharmaceutically acceptable salt, hydrate, solvate, prodrug, stereoisomer, tautomer, or pharmaceutical composition thereof.

The present invention further provides methods of treating a disease or disorder associated with coronavirus infection, comprising administering to a patient suffering from coronavirus infection a compound of Formula (II) or a pharmaceutically acceptable salt, hydrate, solvate, prodrug, stereoisomer, tautomer, or pharmaceutical composition thereof.

The present invention provides inhibitors of SARS-CoV-2-related 3C-like protease that are therapeutic agents in the treatment of coronavirus infection.

The present invention provides inhibitors of SARS-CoV-2-related Mpro protease that are therapeutic agents in the treatment of coronavirus infection.

The present invention further provides compounds and compositions with an improved efficacy and safety profile relative to known SARS-CoV-2-related 3C-like protease inhibitors. The present disclosure also provides agents with novel mechanisms of action toward SARS-CoV-2-related 3C-like protease in the treatment of coronavirus infection.

The present invention further provides compounds and compositions with an improved efficacy and safety profile relative to known SARS-CoV-2-related Mpro protease inhibitors. The present disclosure also provides agents with novel mechanisms of action toward SARS-CoV-2-related Mpro protease in the treatment of coronavirus infection.

The present invention further provides methods of preventing, treating, or ameliorating a coronavirus infection.

The present invention further provides methods of treating a disease, disorder, or condition selected from cold; pneumonia (either direct viral pneumonia or secondary bacterial pneumonia); bronchitis (either direct viral bronchitis or secondary bacterial bronchitis); severe acute respiratory syndrome (SARS); Middle East respiratory syndrome (MERS); Coronavirus disease 2019 (COVID-19); Coronavirus HuPn-2018 comprising administering to a patient suffering from at least one of said diseases or disorders a compound of Formula (A), or a pharmaceutically acceptable salt, hydrate, solvate, prodrug, stereoisomer, tautomer, or pharmaceutical composition thereof.

The present invention further provides methods of treating a disease, disorder, or condition selected from cold; pneumonia (either direct viral pneumonia or secondary bacterial pneumonia); bronchitis (either direct viral bronchitis or secondary bacterial bronchitis); severe acute respiratory syndrome (SARS); Middle East respiratory syndrome (MERS); Coronavirus disease 2019 (COVID-19); Coronavirus HuPn-2018 comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease, disorder, or condition selected from cold; pneumonia (either direct viral pneumonia or secondary bacterial pneumonia); bronchitis (either direct viral bronchitis or secondary bacterial bronchitis); severe acute respiratory syndrome (SARS); Middle East respiratory syndrome (MERS); Coronavirus disease 2019 (COVID-19); Coronavirus HuPn-2018 comprising administering to a patient suffering from at least one of said diseases or disorders a compound of Formula (II), or a pharmaceutically acceptable salt, hydrate, solvate, prodrug, stereoisomer, tautomer, or pharmaceutical composition thereof.

In some aspects, the present disclosure provides a compound obtainable by, or obtained by, a method for preparing compounds described herein (e.g., a method comprising one or more steps described in General Procedures).

In some aspects, the present disclosure provides an intermediate as described herein, being suitable for use in a method for preparing a compound as described herein (e.g., the intermediate is selected from the intermediates described in Preparative part—Preparations 1-278).

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims

### DETAILED DESCRIPTION

The present disclosure provides methods of treating, preventing, or ameliorating a viral infection associated with coronavirus by administering to a subject in need thereof a therapeutically effective amount of a compound as disclosed herein.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have one or more substituents different from hydrogen. For instance, it can, at any point along the chain be bonded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —NH₂, —NO₂, —COOH, —CH₂CN, —O—(C₁-C₆) alkyl, (C₁-C₆) alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OP(O)(OH)₂, —OC(O)(C₁-C₆) alkyl, —C(O)(C₁-C₆) alkyl, —OC(O)O(C₁-C₆) alkyl, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆)alkyl, and —S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆)alkyl, (C₁-C₆)alkyl, —O—(C₂-C₆)alkenyl, —O—(C₂-C₆) alkynyl, (C₂-C₆)alkenyl, (C₂-C₆)alkynyl, —OH, —OP(O)(OH)₂, —OC(O)(C₁-C₆)alkyl, —C(O)(C₁-C₆) alkyl, —OC(O)O(C₁-C₆)alkyl, —NH₂, —NH((C₁-C₆)alkyl), —N((C₁-C₆)alkyl)₂, —S(O)₂—(C₁-C₆) alkyl, —S(O)NH(C₁-C₆)alkyl, and —S(O)N((C₁-C₆)alkyl)₂. The substituents can themselves be optionally substituted. Furthermore, when containing two fused rings the aryl groups herein defined may have one or more saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or a polycyclic aromatic radical of 5 to 24 ring atoms, containing one or more ring heteroatoms selected from N, O, S, P, or B, the remaining ring atoms being C. A polycyclic aromatic radical includes two or more fused rings and may further include two or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B. Heteroaryl as herein defined also means a tricyclic heteroaromatic group containing one or more ring heteroatoms selected from N, O, S, P, or B. Heteroaryl as herein defined also means a tetracyclic heteroaromatic group containing one or more ring heteroatoms selected from N, O, S, P, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo [1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo [1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with one or more fully unsaturated aromatic ring. In heteroaryl ring systems containing more than two fused rings, a saturated or partially unsaturated ring may further be fused with a saturated or partially unsaturated ring described herein. Furthermore, when containing three or more fused rings, the heteroaryl groups defined herein may have one or more saturated or partially unsaturated ring spiro-fused. Any saturated or partially unsaturated ring described herein is optionally substituted with one or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]puyrrolo[1,2-d][1,4]oxazin-9-onyl, or 6a′,7′-dihydro-6′H,9′H-spiro[cyclopropane-1,8′-pyrido[2,3-b]pyrrolo[1,2-d][1,4]oxazin]-9′-onyl.

Halogen or “halo” refers to fluorine, chlorine, bromine, or iodine.

Alkyl refers to a straight or branched chain saturated hydrocarbon containing 1-12 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” refers to a straight or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, tert-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkynyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means mono or polycyclic saturated carbon rings containing 3-18 carbon atoms. Polycyclic cycloalkyl may be fused bicyclic cycloalkyl, bridged bicyclic cycloalkyl, or spiro-fused bicyclic cycloalkyl. A polycyclic cycloalkyl comprises at least one non-aromatic ring. Examples of cycloalkyl groups include, without limitations, cyclopropyl, cyclobutyl, cyclopentyl, cyclohexyl, cycloheptanyl, cyclooctanyl, norbornyl, norbornenyl, 1,2,3,4-tetrahydronaphthyl, 2,3-dihydro-1H-indenyl, spiro[3.5]nonyl, spiro [5.5]undecyl, bicyclo[1.1.1]pentanyl, bicyclo[2.2.2]octanyl, or bicyclo[2.2.2]octenyl.

“Heterocyclyl”, “heterocycle” or “heterocycloalkyl” mono or polycyclic rings containing 3-24 atoms which include carbon and one or more heteroatoms selected from N, O, S, P, or B and wherein the rings are not aromatic. The heterocycloalkyl ring structure may be substituted by one or more substituents. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “aromatic” means a planar ring having 4n+2 electrons in a conjugated system. As used herein, “conjugated system” means a system of connected p-orbitals with delocalized electrons, and the system may include lone electron pairs.

The term “halogenalkyl” as used herein refers to an alkyl group, as defined herein, which is substituted one or more halogen. Examples of halogenalkyl groups include, but are not limited to, trifluoromethyl, difluoromethyl, pentafluoroethyl, trichloromethyl, etc.

The term “halogenalkoxy” as used herein refers to an alkoxy group, as defined herein, which is substituted with one or more halogen. Examples of haloalkyl groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C≡N.

“Spirocycloalkyl” or “spirocyclyl” means carbogenic bicyclic ring systems with both rings connected through a single atom. The ring can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing between 3 and 12 carbon atoms. One or more of the carbon atoms can be substituted with a heteroatom.

The term “spiroheterocycloalkyl”, “spiroheterocycle”, or “spiroheterocyclyl” is understood to mean a spirocycle wherein at least one of the rings is a heterocycle (e.g., at least one of the rings is furanyl, morpholinyl, or piperidinyl).

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (A) may have one or more asymmetric carbon atom and may occur as racemates, racemic mixtures and as individual enantiomers or diastereomers.

The present disclosure also contemplates isotopically-labelled compounds of Formula (A) (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising a therapeutically effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents, and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound.

The term “salt” refers to pharmaceutically acceptable salts.

The term “pharmaceutically acceptable salt” also refers to a salt of the compositions of the present disclosure having an acidic functional group, such as a carboxylic acid functional group, and a base.

“SARS-CoV-2-related 3C-like protease inhibitor” as used herein refer to compounds of Formula (A), Formula (I) or Formula (II) and/or compositions comprising a compound of Formula (A), Formula (I), or Formula (II) which inhibit of SARS-CoV-2-related 3C-like protease.

“SARS-CoV-2 viral replication inhibitor” as used herein refer to compounds of Formula (A), Formula (I), or Formula (II) and/or compositions comprising a compound of Formula (A), Formula (I), or Formula (II) which inhibit of SARS-CoV-2 viral replication.

The term “SARS-CoV-2 inhibiting agent” means any SARS-CoV-2-related coronavirus 3C-like protease inhibitor compound described herein or a pharmaceutically acceptable salt, hydrate, prodrug, active metabolite or solvate thereof or a compound which inhibits replication of SARS-CoV-2 in any manner.

The amount of compound or composition described herein needed for achieving a therapeutic effect may be determined empirically in accordance with conventional procedures for the particular purpose. Generally, for administering therapeutic agents (e.g. compounds or compositions of the disclosure (and/or additional agents) described herein) for therapeutic purposes, the therapeutic agents are given at a pharmacologically effective dose.

A “pharmacologically effective amount”, “pharmacologically effective dose”, “therapeutically effective amount”, or “effective amount” refers to an amount sufficient to produce the desired physiological effect or amount capable of achieving the desired result, particularly for treating the coronavirus infection. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

### Compounds of the Present Disclosure

In one aspect, the present disclosure provides compounds of Formula (A) and pharmaceutically acceptable salts, stereoisomers, solvates, prodrugs, isotopic derivatives, and tautomers thereof:

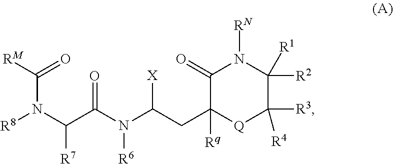

Wherein R^{N}, R^{q}, R^{M}, R¹, R², R³, R⁴, R⁶, R⁷, R⁸, Q, and X are as described herein.

It is understood that, for a compound of Formula (A), R^{N}, R^{q}, R^{M}, R¹, R², R³, R⁴, R⁶, R⁷, R⁸, Q, and X can each be, where applicable, selected from the groups described herein, and any group described herein for any of R^{N}, R^{q}, R^{M}, R¹, R², R³, R⁴, R⁶, R⁷, R⁸, Q, and X can be combined, where applicable, with any group described herein for one or more of the remainder of R^{N}, R^{q}, R^{M}, R¹, R², R³, R⁴, R⁶, R⁷, R⁸, Q, and X.

In another aspect, the present disclosure provides compounds of Formula (I) and pharmaceutically acceptable salts, stereoisomers, solvates, prodrugs, isotopic derivatives, and tautomers thereof:

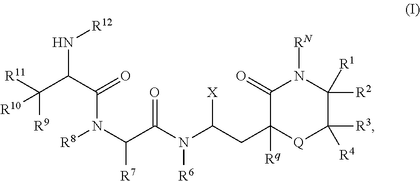

Wherein R^{N}, R^{q}, R¹, R², R³, R⁴, R⁵, R⁶, R⁷, R⁸, R⁹, R¹⁰, R¹¹, R¹², Q, and X are as described herein.

It is understood that, for a compound of Formula (I), R^{N}, R^{q}, R¹, R², R³, R⁴, R⁵, R⁶, R⁷, R⁸, R⁹, R¹⁰, R¹¹, R¹², Q, and X can each be, where applicable, selected from the groups described herein, and any group described herein for any of R^{N}, R^{q}, R¹, R², R³, R⁴, R⁵, R⁶, R⁷, R⁸, R⁹, R¹⁰, R¹¹, R¹², Q, and X can be combined, where applicable, with any group described herein for one or more of the remainder of R^{N}, R^{q}, R¹, R², R³, R⁴, R⁵, R⁶, R⁷, R⁸, R⁹, R¹⁰, R¹¹, R¹², Q, and X.

In another aspect, the present disclosure provides compounds of Formula (II) and pharmaceutically acceptable salts, stereoisomers, solvates, prodrugs, isotopic derivatives, and tautomers thereof:

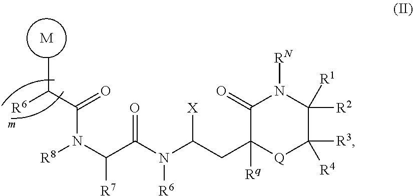

Wherein R^{N}, R^{q}, R¹, R², R³, R⁴, R⁶, R⁷, R⁸, Q, M, m, and X are as described herein.

It is understood that, for a compound of Formula (II), R^{N}, R^{q}, R¹, R², R³, R⁴, R⁶, R⁷, R⁸, Q, M, m, and X can each be, where applicable, selected from the groups described herein, and any group described herein for any of R^{N}, R^{q}, R¹, R², R³, R⁴, R⁶, R⁷, R⁸, Q, M, m, and X can be combined, where applicable, with any group described herein for one or more of the remainder of R^{N}, R^{q}, R¹, R², R³, R⁴, R⁶, R⁷, R⁸, Q, M, m, and X.

In some embodiments,
each R^{N} is independently selected from H, C₁-C₆ alkyl, cycloalkyl;
Q is CR⁵R^{Q} or NR^{Q};
R^{q} is H;
R^{Q} is R^{N}; or
R^{q} and R^{Q} together form a bond;
R^{M} is selected from

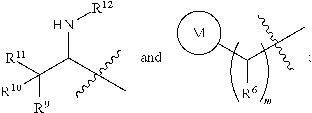

M is selected from 5-10 membered heterocyclyl or 5-10 membered heteroaryl, wherein the heterocyclyl or heteroaryl is optionally substituted with one or more R¹⁶;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R² together are oxo;
or R² and R³ together form a double bond;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
each R⁶ is independently selected from H, C₁-C₆ alkyl, cycloalkyl, —CH₂-Aryl, wherein the alkyl, cycloalkyl or aryl is optionally substituted with one or more R¹⁶;
R⁷ is selected from —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
or R⁷ and R⁸ together with the atoms to which they are attached and any intervening atoms, form 5-7 membered monocyclic or bicyclic heterocyclyl optionally substituted with one or more R³;
R⁹, R¹⁰, R¹¹ are each independently selected from C₁-C₆ alkyl, cycloalkyl, halogen, —CN;
R¹² is selected from R⁶—S(O)₂—, R⁶—C(O)—, R⁶—O—C(O)—, heteroaryl, wherein the heteroaryl is optionally substituted with one or more R¹⁶;
each R¹³ is independently selected from halogen, —OH, —CN, —C₁-C₆ alkyl, —C₁-C₆ halogenalkyl; or
two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
R¹⁴ and R¹⁵ are each independently selected from H, C₁-C₆ alkyl, cycloalkyl;
or R¹⁴ and R¹⁵ together with the atoms to which they are attached and any intervening atoms, form heterocyclyl optionally substituted with one or more halogen, —OH, —CN, C₁-C₆ alkyl;
each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
n is an integer selected from 0, 1, 2;
m is an integer selected from 0 and 1.

In some embodiments, Q is selected from CR⁵R^{Q} and NR^{Q}.

In some embodiments, Q is CR⁵R^{Q}.

In some embodiments, Q is NR^{Q}.

In some embodiments, R^{Q} is R^{N}.

In some embodiments, R^{Q} is H.

In some embodiments, R^{q} is H.

In some embodiments, R^{q} and R^{Q} together form a bond.

In some embodiments, R^{q} and R^{Q} together form an additional bond between C(R^{q}) and Q.

In some embodiments, R^{q} and R^{Q} together form a second bond between C(R^{q}) and Q.

In some embodiments, R^{N} is selected from H, C₁-C₆ alkyl, cycloalkyl.

In some embodiments, R^{N} is H.

In some embodiments, R^{N} is C₁-C₆ alkyl.

In some embodiments, R^{N} is CH₃.

In some embodiments, R^{N} is cycloalkyl.

In some embodiments, R^{N} is cyclopropyl.

In some embodiments, R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R¹ is hydrogen.

In some embodiments, R¹ and R² together are oxo.

In some embodiments, R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶.

In some embodiments, R¹ and R⁴ together with the atoms to which they are attached and any intervening atoms, form a 3-10 membered cycloalkyl optionally substituted with one or more R¹⁶.

In some embodiments, R¹ and R⁴ together with the atoms to which they are attached and any intervening atoms, form a 6 membered cycloalkyl optionally substituted with one or more R¹⁶.

In some embodiments, R¹ and R⁴ together with the atoms to which they are attached and any intervening atoms, form a 3-10 membered cycloalkyl optionally substituted with one or more R¹⁶ and R² and R³ together form a double bond.

In some embodiments, R¹ and R⁴ together with the atoms to which they are attached and any intervening atoms, form a 3-10 membered unsubstituted cycloalkyl and R² and R³ together form a double bond.

In some embodiments, R¹ and R⁴ together with the atoms to which they are attached and any intervening atoms, form a 6 membered cycloalkyl optionally substituted with one or more R¹⁶ and R² and R³ together form a double bond.

In some embodiments, R¹ and R⁴ together with the atoms to which they are attached and any intervening atoms, form a 6 membered unsubstituted cycloalkyl and R² and R³ together form a double bond.

In some embodiments, R¹ and R⁴ together with the atoms to which they are attached and any intervening atoms, form a 6 membered cycloalkyl, R² and R³ together form a double bond and the 6 membered cycloalkyl is substituted with two C₁-C₆ alkyl.

In some embodiments, R¹ and R⁴ together with the atoms to which they are attached and any intervening atoms, form a 6 membered cycloalkyl, R² and R³ together form a double bond and the 6 membered cycloalkyl is substituted with two methyl.

In some embodiments, R¹ and R⁴ together with the atoms to which they are attached and any intervening atoms, form a 6 membered cycloalkyl, R² and R³ together form a double bond and the 6 membered cycloalkyl is substituted with two halogens.

In some embodiments, R¹ and R⁴ together with the atoms to which they are attached and any intervening atoms, form a 6 membered cycloalkyl, R² and R³ together form a double bond and the 6 membered cycloalkyl is substituted with two fluorine.

In some embodiments, R¹ and R⁴ together with the atoms to which they are attached and any intervening atoms, form a 6 membered aryl optionally substituted with one or more R¹⁶.

In some embodiments, R¹ and R⁴ together with the atoms to which they are attached and any intervening atoms, form a 6 membered unsubstituted aryl.

In some embodiments, R¹ and R⁴ together with the atoms to which they are attached and any intervening atoms, form a 6 membered aryl substituted with one R¹⁶.

In some embodiments, R¹ and R⁴ together with the atoms to which they are attached and any intervening atoms, form a 6 membered aryl substituted with two R¹⁶.

In some embodiments, R¹ and R⁴ together with the atoms to which they are attached and any intervening atoms, form a 6 membered aryl substituted with three R¹⁶.

In some embodiments, R¹ and R⁴ together with the atoms to which they are attached and any intervening atoms, form a 6 membered aryl substituted with four R¹⁶.

In some embodiments, R¹ and R⁴ together with the atoms to which they are attached and any intervening atoms, form a 6 membered aryl substituted with one or more halogen.

In some embodiments, R¹ and R⁴ together with the atoms to which they are attached and any intervening atoms, form a 6 membered aryl substituted with one or more F.

In some embodiments, R¹ and R⁴ together with the atoms to which they are attached and any intervening atoms, form a 6 membered aryl substituted with one F.

In some embodiments, R¹ and R⁴ together with the atoms to which they are attached and any intervening atoms, form a 6 membered aryl substituted with one or more Cl.

In some embodiments, R¹ and R⁴ together with the atoms to which they are attached and any intervening atoms, form a 6 membered aryl substituted with one Cl.

In some embodiments, R¹ and R⁴ together with the atoms to which they are attached and any intervening atoms, form a 6 membered aryl optionally with one or more C₁-C₆ alkyl.

In some embodiments, R¹ and R⁴ together with the atoms to which they are attached and any intervening atoms, form a 6 membered aryl substituted with one or more methyl.

In some embodiments, R¹ and R⁴ together with the atoms to which they are attached and any intervening atoms, form a 6 membered aryl substituted with one methyl.

In some embodiments, R¹ and R⁴ together with the atoms to which they are attached and any intervening atoms, form a 6 membered aryl substituted with two methyl.

In some embodiments, R¹ and R⁴ together with the atoms to which they are attached and any intervening atoms, form a 6 membered aryl optionally with one or more C₁-C₆ alkoxy.

In some embodiments, R¹ and R⁴ together with the atoms to which they are attached and any intervening atoms, form a 6 membered aryl substituted with one or more methoxy.

In some embodiments, R¹ and R⁴ together with the atoms to which they are attached and any intervening atoms, form a 6 membered aryl substituted with one methoxy.

In some embodiments, R¹ and R⁴ together with the atoms to which they are attached and any intervening atoms, form a 3-14 membered heterocycle optionally substituted with one or more R¹⁶.

In some embodiments, R¹ and R⁴ together with the atoms to which they are attached and any intervening atoms, form a 6 membered heterocycle optionally substituted with one or more R¹⁶.

In some embodiments, R¹ and R⁴ together with the atoms to which they are attached and any intervening atoms, form a 6 membered heterocycle comprising nitrogen as a heteroatom optionally substituted with one or more R¹⁶.

In some embodiments, R¹ and R⁴ together with the atoms to which they are attached and any intervening atoms, form a 6 membered heterocycle comprising oxygen as a heteroatom optionally substituted with one or more R¹⁶.

In some embodiments, R¹ and R⁴ together with the atoms to which they are attached and any intervening atoms, form a 6 membered heterocycle comprising sulfur as a heteroatom optionally substituted with one or more R¹⁶.

In some embodiments, R¹ and R⁴ together with the atoms to which they are attached and any intervening atoms, form a 5-6 membered heteroaryl optionally substituted with one or more R¹⁶.

In some embodiments, R¹ and R⁴ together with the atoms to which they are attached and any intervening atoms, form a 6 membered heteroaryl optionally substituted with one or more R¹⁶.

In some embodiments, R¹ and R⁴ together with the atoms to which they are attached and any intervening atoms, form a 6 membered heteroaryl comprising nitrogen as a heteroatom optionally substituted with one or more R¹⁶.

In some embodiments, R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R² is H.

In some embodiments, R² and R¹ together are oxo.

In some embodiments, R² and R³ together form a double bond.

In some embodiments, R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, cycloalkyl, aryl, heterocyclyl, or heteroaryl is optionally substituted with one or more R¹⁶.

In some embodiments, R³ and R² together form a double bond.

In some embodiments, R³ is H.

In some embodiments, R⁴ is selected from hydrogen, halogen, —OH, —CN, —NO₂, —NR¹³R¹⁴, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R⁴ is H.

In some embodiments, R⁴ is —OH.

In some embodiments, R⁴ is halogen.

In some embodiments, R⁴ is F.

In some embodiments, R⁴ is C₁-C₆ alkyl.

In some embodiments, R⁴ is methyl. In some embodiments, R⁴ is ethyl. In some embodiments, R⁴ is propyl. In some embodiments, R⁴ is n-propyl. In some embodiments, R⁴ is iso-propyl. In some embodiments, R⁴ is butyl. In some embodiments, R⁴ is n-butyl. In some embodiments, R⁴ is iso-butyl. In some embodiments, R⁴ is n-butyl. In some embodiments, R⁴ is sec-butyl. In some embodiments, R⁴ is tert-butyl. In some embodiments, R⁴ is pentyl. In some embodiments, R⁴ is n-pentyl. In some embodiments, R⁴ is n-hexyl.

In some embodiments, R⁴ is CH₃.

In some embodiments, R⁴ is C₁-C₆ alkoxy.

In some embodiments, R⁴ is methoxy. In some embodiments, R⁴ is ethoxy. In some embodiments, R⁴ is propoxy. In some embodiments, R⁴ is —OCH₂CH₂CH₃. In some embodiments, R⁴ is —OCH(CH₃)₂. In some embodiments, R⁴ is butoxy. In some embodiments, R⁴ is —OCH₂CH₂CH₂CH₃. In some embodiments, R⁴ is —OCH₂CH(CH₃)₂. In some embodiments, R⁴ is —OC(CH₃)₃. In some embodiments, R⁴ is pentoxy. In some embodiments, R⁴ is hexoxy.

In some embodiments, R⁴ is —OCH₃.

In some embodiments, R⁴ is —OC₂H₅.

In some embodiments, R⁴ is —O—CH₂CH₂CH₃.

In some embodiments, R⁴ is

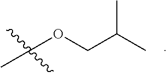 [SMILES: *OCC(C)C]

In some embodiments, R⁴ is

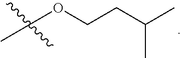 [SMILES: *OCCC(C)C]

In some embodiments, R⁴ is —NR⁶R⁷.

In some embodiments, R⁴ is —NH₂.

In some embodiments, R⁴ is —NHCH₃.

In some embodiments, R⁴ is —N(CH₃)₂.

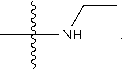 [SMILES: *NCC]

In some embodiments, R⁴ is

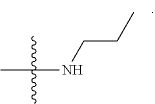 [SMILES: *NCCC]

In some embodiments, R⁴ is

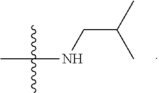 [SMILES: *NCC(C)C]

In some embodiments, R⁴ is

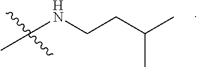 [SMILES: *NCCC(C)C]

In some embodiments, R⁴ is —N(CH₃)₂.

In some embodiments, R⁴ is

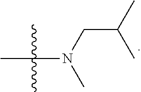 [SMILES: *N(C)CC(C)C]

In some embodiments, R⁴ is

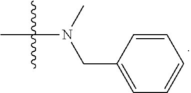 [SMILES: *N(C)Cc1ccccc1]

In some embodiments, R⁴ is heterocyclyl.

In some embodiments, R⁴ is

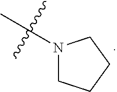 [SMILES: *N1CCCC1]

In some embodiments, R⁴ is

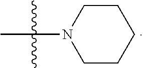 [SMILES: *N1CCCCC1]

In some embodiments, R⁴ is C₁-C₆ alkoxy substituted with one group R⁵.

In some embodiments, R⁴ is C₁-C₆ alkoxy substituted with one cycloalkyl.

In some embodiments, R⁴ is

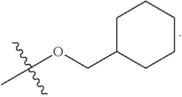 [SMILES: *OCC1CCCCC1]

In some embodiments, R⁴ and R¹ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶.

In some embodiments, R⁴ and R¹ together with the atoms to which they are attached and any intervening atoms, form a 6 membered aryl is optionally substituted with one or more R¹⁶.

In some embodiments, R⁴ and R¹ together with the atoms to which they are attached and any intervening atoms, form a 6 membered unsubstituted aryl.

In some embodiments, R⁴ and R¹ together with the atoms to which they are attached and any intervening atoms, form a 6 membered aryl substituted with one or more halogen.

In some embodiments, R⁴ and R¹ together with the atoms to which they are attached and any intervening atoms, form a 6 membered aryl substituted with one or more F.

In some embodiments, R⁴ and R¹ together with the atoms to which they are attached and any intervening atoms, form a 6 membered aryl substituted with one F.

In some embodiments, R⁴ and R¹ together with the atoms to which they are attached and any intervening atoms, form a 6 membered aryl substituted with one or more Cl.

In some embodiments, R⁴ and R¹ together with the atoms to which they are attached and any intervening atoms, form a 6 membered aryl substituted with one Cl.

In some embodiments, R⁴ and R¹ together with the atoms to which they are attached and any intervening atoms, form a 6 membered aryl optionally with one or more C₁-C₆ alkyl.

In some embodiments, R⁴ and R¹ together with the atoms to which they are attached and any intervening atoms, form a 6 membered aryl substituted with one or more methyl.

In some embodiments, R⁴ and R¹ together with the atoms to which they are attached and any intervening atoms, form a 6 membered aryl substituted with one methyl.

In some embodiments, R⁴ and R¹ together with the atoms to which they are attached and any intervening atoms, form a 6 membered aryl optionally with one or more C₁-C₆ alkoxy.

In some embodiments, R⁴ and R¹ together with the atoms to which they are attached and any intervening atoms, form a 6 membered aryl substituted with one or more methoxy.

In some embodiments, R⁴ and R¹ together with the atoms to which they are attached and any intervening atoms, form a 6 membered aryl substituted with one methoxy.

In some embodiments, R⁵ is selected from hydrogen, deuterium, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹³, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R⁵ is hydrogen.

In some embodiments, R⁵ is C₁-C₆ alkyl.

In some embodiments, R⁶ is selected from H, C₁-C₆ alkyl, cycloalkyl, —CH₂-Aryl.

In some embodiments, R⁶ is H.

In some embodiments, R⁶ is C₁-C₆ alkyl.

In some embodiments, R⁶ is methyl.

In some embodiments, R⁶ is ethyl.

In some embodiments, R⁶ is propyl.

In some embodiments, R⁶ is n-propyl.

In some embodiments, R⁶ is iso-propyl.

In some embodiments, R⁶ is cycloalkyl.

In some embodiments, R⁷ is selected from —(CH₂)ₙaryl, wherein aryl is optionally substituted with one or more substitutents independently selected from halogen, —OH, —CN, —NO₂, —NR¹³R¹⁴, C₁-C₆ alkyl, C₂-C₆ alkenyl, C₂-C₆ alkynyl, C₁-C₆ alkoxy, cycloalkyl, aryl, heterocyclyl, and heteroaryl.

In some embodiments, n is an integer selected from 0, 1, 2.

In some embodiments, n is 2.

In some embodiments, n is 1.

In some embodiments, n is 0.

In some embodiments, R⁷ is —(CH₂)ₙaryl, wherein aryl is optionally substituted with one or more substitutents independently selected from halogen, —OH, —CN, —NO₂, —NR¹³R¹⁴, C₁-C₆ alkyl, C₂-C₆ alkenyl, C₂-C₆ alkynyl, C₁-C₆ alkoxy, cycloalkyl, aryl, heterocyclyl, and heteroaryl.

In some embodiments, R⁷ is —CH₂-aryl, wherein aryl is optionally substituted with one or more substitutents independently selected from halogen, —OH, —CN, —NO₂, —NR¹³R¹⁴, C₁-C₆ alkyl, C₂-C₆ alkenyl, C₂-C₆ alkynyl, C₁-C₆ alkoxy, cycloalkyl, aryl, heterocyclyl, and heteroaryl.

In some embodiments, R⁷ is —CH₂-phenyl.

In some embodiments, R⁷ is —CH₂-aryl, wherein aryl is optionally substituted with one or more halogen.

In some embodiments, R⁷ is

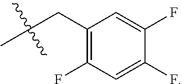 [SMILES: *Cc1cc(F)c(F)cc1F]

In some embodiments, R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl.

In some embodiments, R⁸ is hydrogen.

In some embodiments, R⁸ is C₁-C₆ alkyl.

In some embodiments, R⁸ is methyl.

In some embodiments, R⁷ and R⁸ together with the atoms to which they are attached and any intervening atoms, form 5-7 membered monocyclic or bicyclic heterocyclyl optionally substituted with one or more R¹³.

In some embodiments, R⁷ and R⁸ together with the atoms to which they are attached and any intervening atoms, form 5 membered monocyclic heterocyclyl optionally substituted with one or more R¹³.

In some embodiments, R⁷ and R⁸ together with the atoms to which they are attached and any intervening atoms, form 6 membered monocyclic heterocyclyl optionally substituted with one or more R¹³.

In some embodiments, R⁷ and R⁸ together with the atoms to which they are attached and any intervening atoms, form 7 membered monocyclic heterocyclyl optionally substituted with one or more R¹³.

In some embodiments, R⁷ and R⁸ together with the atoms to which they are attached and any intervening atoms, form 6 membered bicyclic heterocyclyl optionally substituted with one or more R¹³.

In some embodiments, R⁷ and R⁸ together with the atoms to which they are attached and any intervening atoms, form 7 membered bicyclic heterocyclyl optionally substituted with one or more R¹³.

In some embodiments, R⁷ and R⁸ together with the atoms to which they are attached and any intervening atoms, form

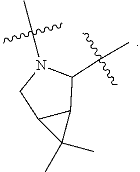 [SMILES: *C1C2C(CN1*)C2(C)C]

In some embodiments, R⁷ and R⁸ together with the atoms to which they are attached and any intervening atoms, form

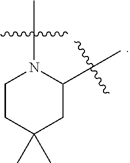 [SMILES: *C1CC(C)(C)CCN1*]

In some embodiments R⁹, R¹⁰, R¹¹ are each independently selected from C₁-C₆ alkyl, cycloalkyl, halogen, —CN.

In some embodiments R⁹, R¹⁰, R¹¹ are each C₁-C₆ alkyl.

In some embodiments R⁹, R¹⁰, R¹¹ are each methyl.

In some embodiments R⁹, R¹⁰, R¹¹ are each halogen.

In some embodiments R⁹, R¹⁰, R¹¹ are each F.

In some embodiments R⁹ is methyl, R¹⁰ and R¹¹ are each F.

In some embodiments R⁹ is F, R¹⁰ and R¹¹ are each methyl.

In some embodiments, R¹² is selected from R⁶—S(O)₂—, R⁶—C(O)—, R⁶—O—C(O)—, heteroaryl, wherein the heteroaryl is optionally substituted with one or more R¹⁶.

In some embodiments, R¹² is C₁-C₆ alkyl-S(O)₂—, wherein the alkyl is optionally substituted with one or more R¹⁶.

In some embodiments, R¹² is CH₃S(O)₂—.

In some embodiments, R¹² is CF₃S(O)₂—.

In some embodiments, R¹² is CF₃CH₂S(O)₂—.

In some embodiments, R¹² is cycloalkyl-S(O)₂—, wherein the alkyl is optionally substituted with one or more R¹⁶.

In some embodiments, R¹² is

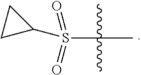 [SMILES: *S(=O)(=O)C1CC1]

In some embodiments, R¹² is C₁-C₆ alkyl-C(O)—, wherein the alkyl is optionally substituted with one or more R¹⁶.

In some embodiments, R¹² is CF₃C(O)—.

In some embodiments, R¹² is (CH₃)₂CHC(O)—.

In some embodiments, R¹² is C₁-C₆ alkyl-O—C(O)—, wherein the alkyl is optionally substituted with one or more R¹⁶.

In some embodiments, R¹² is (CH₃)₃C—O—C(O)—.

In some embodiments, R¹² is CH₃OC(O)—.

In some embodiments, R¹² is CH₃CH₂OC(O)—.

In some embodiments, R¹² is (CH₃)₂CH—OC(O)—.

In some embodiments, R¹² is heteroaryl.

In some embodiments, R¹² is 5 membered heteroaryl.

In some embodiments, R¹² is 6 membered heteroaryl.

In some embodiments, R¹² is 5 membered heteroaryl comprising 1 heteroatom.

In some embodiments, R¹² is 5 membered heteroaryl comprising 2 heteroatoms.

In some embodiments, R¹² is 5 membered heteroaryl comprising 3 heteroatoms.

In some embodiments, R¹² is 6 membered heteroaryl comprising 1 heteroatom.

In some embodiments, R¹² is 6 membered heteroaryl comprising 2 heteroatoms.

In some embodiments, R¹² is 6 membered heteroaryl comprising 3 heteroatoms.

In some embodiments, R¹² is 5 membered heteroaryl comprising 1 heteroatom selected from N, O, S.

In some embodiments, R¹² is 5 membered heteroaryl comprising 2 heteroatoms, independently selected from N, O, S.

In some embodiments, R¹² is 5 membered heteroaryl comprising 3 heteroatoms, independently selected from N, O, S.

In some embodiments, R¹² is 6 membered heteroaryl comprising 1 heteroatom, selected from N, O, S.

In some embodiments, R¹² is 6 membered heteroaryl comprising 2 heteroatoms, independently selected from N, O, S.

In some embodiments, R¹² is 6 membered heteroaryl comprising 3 heteroatoms, independently selected from N, O, S.

In some embodiments, R¹² is 5 membered heteroaryl comprising 1 heteroatom selected from N, O, S, substituted with one R¹⁶.

In some embodiments, R¹² is 5 membered heteroaryl comprising 2 heteroatoms, independently selected from N, O, S, substituted with one R¹⁶.

In some embodiments, R¹² is 5 membered heteroaryl comprising 3 heteroatoms, independently selected from N, O, S, substituted with one R¹⁶.

In some embodiments, R¹² is 6 membered heteroaryl comprising 1 heteroatom, selected from N, O, S, substituted with one R¹⁶.

In some embodiments, R¹² is 6 membered heteroaryl comprising 2 heteroatoms, independently selected from N, O, S, substituted with one R¹⁶.

In some embodiments, R¹² is 6 membered heteroaryl comprising 3 heteroatoms, independently selected from N, O, S, substituted with one R¹⁶.

In some embodiments, R¹² is

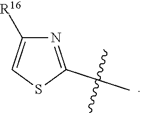

In some embodiments, R¹² is

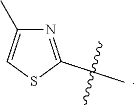 [SMILES: *c1nc(C)cs1]

In some embodiments, R¹² is

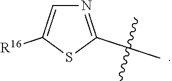

In some embodiments, R¹² is

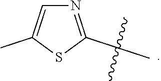 [SMILES: *c1ncc(C)s1]

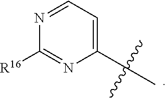

In some embodiments, R¹² is

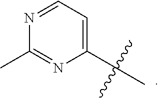 [SMILES: *c1ccnc(C)n1]

In some embodiments, R¹² is

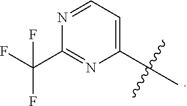 [SMILES: *c1ccnc(C(F)(F)F)n1]

In some embodiments, R¹² is

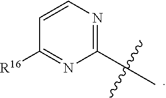

In some embodiments, R¹² is

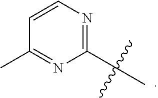 [SMILES: *c1nccc(C)n1]

In some embodiments, R¹² is

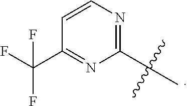 [SMILES: *c1nccc(C(F)(F)F)n1]

In some embodiments, each R¹³ is independently selected from halogen, —OH, —CN, —C₁-C₆ alkyl, —C₁-C₆ halogenalkyl.

In some embodiments, R¹³ is —C₁-C₆ alkyl.

In some embodiments, the compound of Formula (I) comprises two R¹³ which are —C₁-C₆ alkyl.

In some embodiments, the compound of Formula (I) comprises two R¹³ which are methyl.

In some embodiments, two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl.

In some embodiments, two R¹³ together with the atoms to which they are attached and any intervening atoms, form a 5-8 membered cycloalkyl, wherein cycloalkyl is optionally substituted with one or more substituents independently selected from halogen, —OH, —CN, C₁-C₆ alkyl, C₁-C₆ halogenalkyl.

In some embodiments, two R¹³ together with the atoms to which they are attached and any intervening atoms, form a 6 membered aryl, wherein aryl is optionally substituted with one or more substituents independently selected from halogen, —OH, —CN, C₁-C₆ alkyl, C₁-C₆ halogenalkyl.

In some embodiments, two R¹³ together with the atoms to which they are attached and any intervening atoms, form a 5-8 membered heterocycle, wherein heterocycle is optionally substituted with one or more substituents independently selected from halogen, —OH, —CN, C₁-C₆ alkyl, C₁-C₆ halogenalkyl.

In some embodiments, two R¹³ together with the atoms to which they are attached and any intervening atoms, form a 5-6 membered heteroaryl, wherein heteroaryl is optionally substituted with one or more substituents independently selected from halogen, —OH, —CN, C₁-C₆ alkyl, C₁-C₆ halogenalkyl.

In some embodiments, two R¹³ together with the atoms to which they are attached and any intervening atoms, form

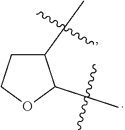 [SMILES: *C1CCOC1*]

In some embodiments, R¹⁴ and R¹⁵ are each independently selected from H, C₁-C₆ alkyl, cycloalkyl.

In some embodiments, R¹⁴ and R¹⁵ are each H.

In some embodiments, R¹⁴ and R¹⁵ are each methyl.

In some embodiments, R¹⁴ and R¹⁵ together with the atoms to which they are attached and any intervening atoms, form heterocyclyl optionally substituted with one or more halogen, —OH, —CN, C₁-C₆ alkyl.

In some embodiments, each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl.

In some embodiments, R¹⁶ is halogen.

In some embodiments, R¹⁶ is F.

In some embodiments, R¹⁶ is Cl.

In some embodiments, R¹⁶ is C₁-C₆ alkyl.

In some embodiments, R¹⁶ is methyl.

In some embodiments, R¹⁶ is ethyl.

In some embodiments, R¹⁶ is propyl.

In some embodiments, R¹⁶ is n-propyl.

In some embodiments, R¹⁶ is iso-propyl.

In some embodiments, R¹⁶ is C₁-C₆ alkoxy.

In some embodiments, R¹⁶ is methoxy.

In some embodiments, R¹⁶ is —C(O)R¹⁴.

In some embodiments, R¹⁶ is —C(O)CH₃.

In some embodiments, X is selected from hydrogen, deuterium, halogen, OH, CN, NO₂, CONR¹⁴R¹⁵, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl.

In some embodiments, X is —CN.

In some embodiments, X is CONR¹⁴R¹⁵.

In some embodiments, X is CONH₂.

In some embodiments, X is selected from C₁-C₆ alkyl optionally substituted with cycloalkyl, heterocyclyl, aryl, and heteroaryl.

In some embodiments, X is selected from C₁-C₆ alkyl substituted with heterocyclyl.

In some embodiments, X is

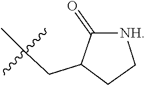 [SMILES: *CC1CCNC1=O]

In some embodiments, X is

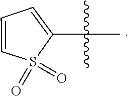 [SMILES: *C1=CC=CS1(=O)=O]

In some embodiments, R^{M} is selected from

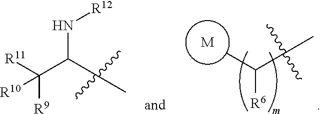

In some embodiments, R^{M} is

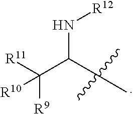

In some embodiments, R^{M} is

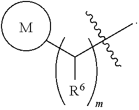

In some embodiments, M is selected from 5-10 membered heterocyclyl or 5-10 membered heteroaryl, wherein the heterocyclyl or heteroaryl is optionally substituted with one or more R¹⁶.

In some embodiments, M is 5-10 membered heterocyclyl optionally substituted with one or more R¹⁶.

In some embodiments, M is 5-10 membered heteroaryl optionally substituted with one or more R¹⁶.

In some embodiments, M is

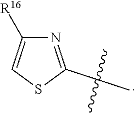

In some embodiments, M is

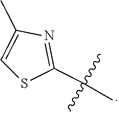 [SMILES: *c1nc(C)cs1]

In some embodiments, M is

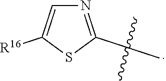

In some embodiments, M is

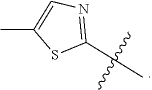 [SMILES: *c1ncc(C)s1]

In some embodiments, M is

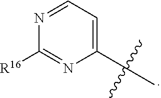

In some embodiments, M is

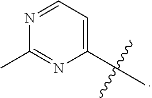 [SMILES: *c1ccnc(C)n1]

In some embodiments, M is

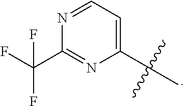 [SMILES: *c1ccnc(C(F)(F)F)n1]

In some embodiments, M is

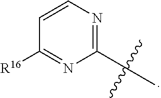

In some embodiments, M is

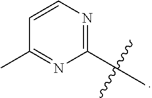 [SMILES: *c1nccc(C)n1]

In some embodiments, M is

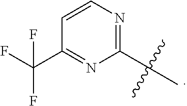 [SMILES: *c1nccc(C(F)(F)F)n1]

In some embodiments, M is

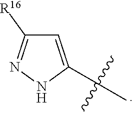

In some embodiments, M is

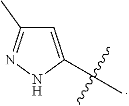 [SMILES: *c1cc(C)n[nH]1]

In some embodiments, M is

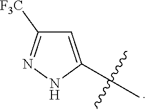 [SMILES: *c1cc(C(F)(F)F)n[nH]1]

In some embodiments, M is

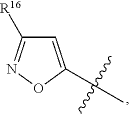

In some embodiments, M is

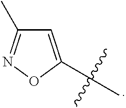 [SMILES: *c1cc(C)no1]

In some embodiments, M is

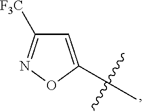 [SMILES: *c1cc(C(F)(F)F)no1]

In some embodiments, M is

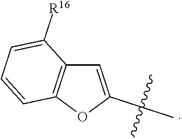

In some embodiments, M is

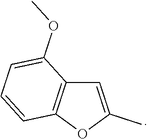

In some embodiments, m is an integer selected from 0 and 1.

In some embodiments, m is 0.

In some embodiments, m is 1.

In some embodiments, the compound is of Formula (I-I):

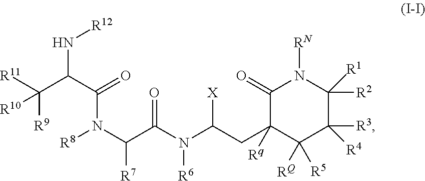

or a pharmaceutically acceptable salt, stereoisomer, solvate, prodrug, isotopic derivative, or tautomer thereof wherein all variables are as defined herein.

In some embodiments, the compound is of Formula (I-II):

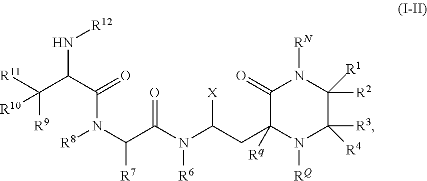

or a pharmaceutically acceptable salt, stereoisomer, solvate, prodrug, isotopic derivative, or tautomer thereof wherein all variables are as defined herein.

In some embodiments, the compound is of Formula (I-I-I):

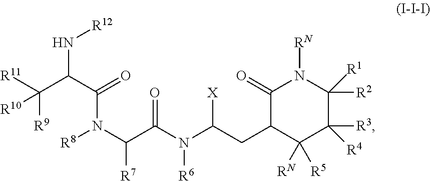

or a pharmaceutically acceptable salt, stereoisomer, solvate, prodrug, isotopic derivative, or tautomer thereof wherein all variables are as defined herein.

In some embodiments, the compound is of Formula (I-I-II):

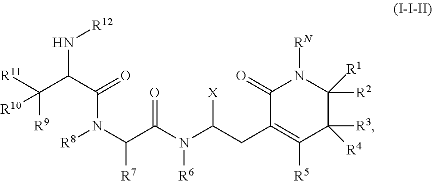

or a pharmaceutically acceptable salt, stereoisomer, solvate, prodrug, isotopic derivative, or tautomer thereof wherein all variables are as defined herein.

In some embodiments, the compound is of Formula (I-II-I):

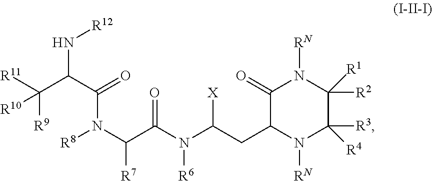

or a pharmaceutically acceptable salt, stereoisomer, solvate, prodrug, isotopic derivative, or tautomer thereof wherein all variables are as defined herein.

In some embodiments, the compound is of Formula (I-II-II):

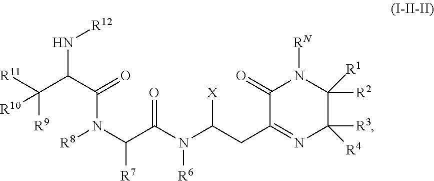

or a pharmaceutically acceptable salt, stereoisomer, solvate, prodrug, isotopic derivative, or tautomer thereof wherein all variables are as defined herein.

In some embodiments, the compound is of Formula (I-I-I-A):

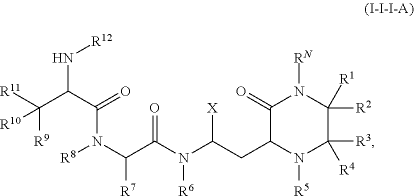

or a pharmaceutically acceptable salt, stereoisomer, solvate, prodrug, isotopic derivative, or tautomer thereof wherein all variables are as defined herein.

In some embodiments, the compound is of Formula (I-I-I-A-I):

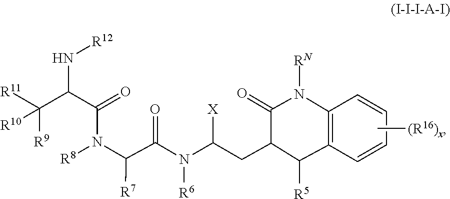

or a pharmaceutically acceptable salt, stereoisomer, solvate, prodrug, isotopic derivative, or tautomer thereof wherein x is an integer selected from 0, 1, 2, 3, and 4 and all other variables are as defined herein.

In some embodiments, the compound is of Formula (I-I-I-A-I-a):

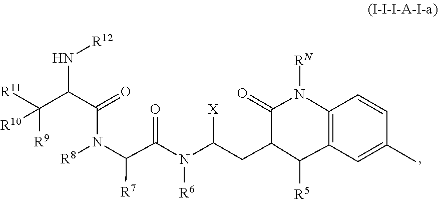

or a pharmaceutically acceptable salt, stereoisomer, solvate, prodrug, isotopic derivative, or tautomer thereof.

In some embodiments, the compound is of Formula (I-I-I-A-I-1):

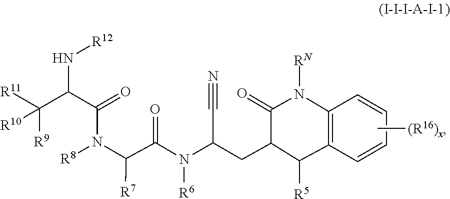

or a pharmaceutically acceptable salt, stereoisomer, solvate, prodrug, isotopic derivative, or tautomer thereof wherein x is an integer selected from 0, 1, 2, 3, 4 and all other variables are as defined herein.

In some embodiments, the compound is of Formula (I-I-I-A-I-1-a):

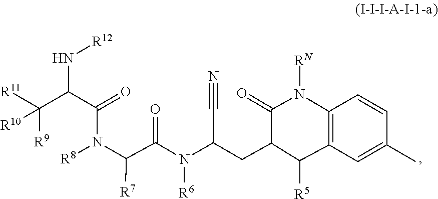

or a pharmaceutically acceptable salt, stereoisomer, solvate, prodrug, isotopic derivative, or tautomer thereof.

In some embodiments, the compound is of Formula (I-I-I-A-I-1-A):

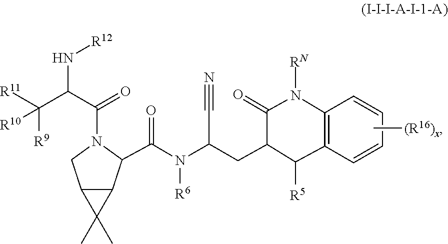

or a pharmaceutically acceptable salt, stereoisomer, solvate, prodrug, isotopic derivative, or tautomer thereof wherein x is an integer selected from 0, 1, 2, 3, 4 and all other variables are as defined herein.

In some embodiments, the compound is of Formula (I-I-I-A-I-1-A*):

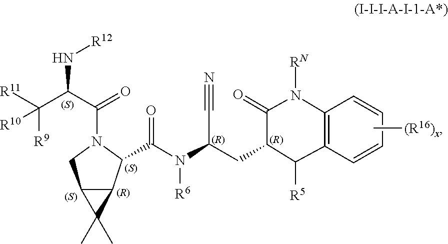

or a pharmaceutically acceptable salt, stereoisomer, solvate, prodrug, isotopic derivative, or tautomer thereof wherein x is an integer selected from 0, 1, 2, 3, 4 and all other variables are as defined herein.

In some embodiments, the compound is of Formula (I-I-I-A-I-1-A**):

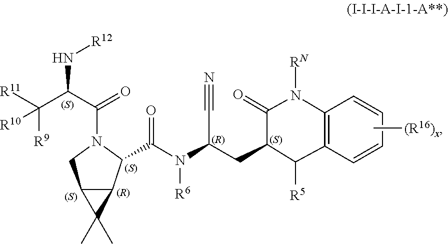

or a pharmaceutically acceptable salt, stereoisomer, solvate, prodrug, isotopic derivative, or tautomer thereof wherein x is an integer selected from 0, 1, 2, 3, 4 and all other variables are as defined herein.

In some embodiments, the compound is of Formula (I-A-I-1-A***):

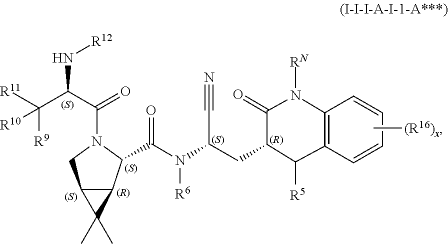

or a pharmaceutically acceptable salt, stereoisomer, solvate, prodrug, isotopic derivative, or tautomer thereof wherein x is an integer selected from 0, 1, 2, 3, 4 and all other variables are as defined herein.

In some embodiments, the compound is of Formula (I-I-I-A-I-1-A****):

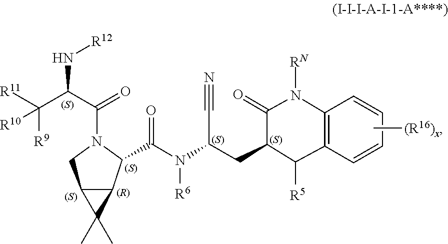

or a pharmaceutically acceptable salt, stereoisomer, solvate, prodrug, isotopic derivative, or tautomer thereof wherein x is an integer selected from 0, 1, 2, 3, 4 and all other variables are as defined herein.

In some embodiments, the compound is of Formula (I-I-I-A-I-1-A-a):

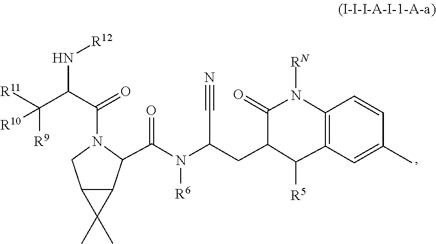

or a pharmaceutically acceptable salt, stereoisomer, solvate, prodrug, isotopic derivative, or tautomer thereof.

In some embodiments, the compound is of Formula (I-I-I-A-I-1-A-a*):

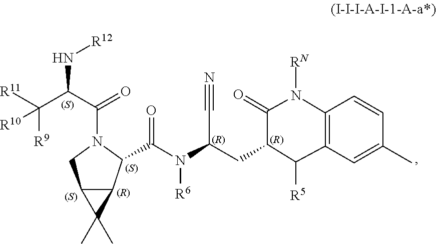

or a pharmaceutically acceptable salt, stereoisomer, solvate, prodrug, isotopic derivative, or tautomer thereof wherein all variables are as defined herein.

In some embodiments, the compound is of Formula (I-I-I-A-I-1-A-a**):

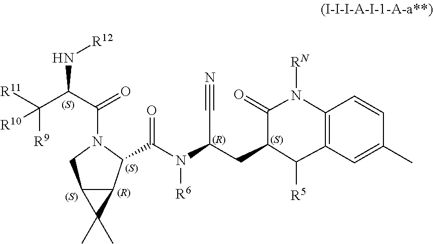

or a pharmaceutically acceptable salt, stereoisomer, solvate, prodrug, isotopic derivative, or tautomer thereof wherein all variables are as defined herein.

In some embodiments, the compound is of Formula (I-I-I-A-I-1-A-a***):

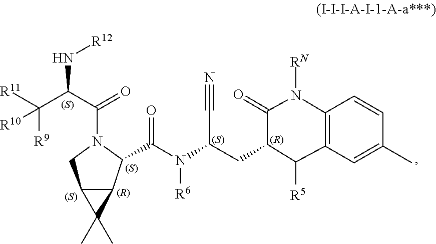

or a pharmaceutically acceptable salt, stereoisomer, solvate, prodrug, isotopic derivative, or tautomer thereof wherein all variables are as defined herein.

In some embodiments, the compound is of Formula (I-I-I-A-I-1-A-a****):

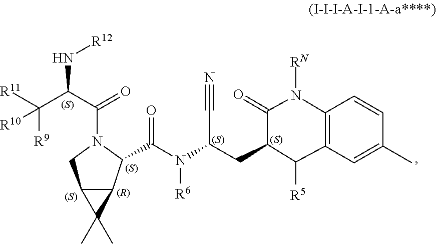

or a pharmaceutically acceptable salt, stereoisomer, solvate, prodrug, isotopic derivative, or tautomer thereof wherein all variables are as defined herein.

In some embodiments, the compound is of Formula (I-I-I-A-I-a-1-A-1):

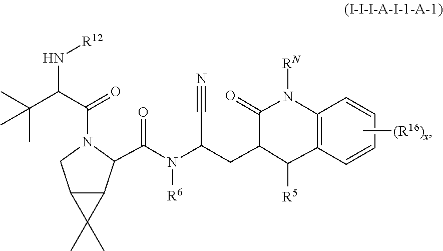

or a pharmaceutically acceptable salt, stereoisomer, solvate, prodrug, isotopic derivative, or tautomer thereof wherein x is an integer selected from 0, 1, 2, 3, 4 and all other variables are as defined herein.

In some embodiments, the compound is of Formula (I-I-I-A-I-a-1-A-1-a):

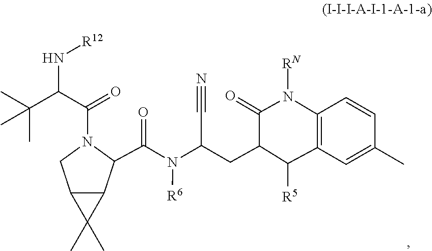

or a pharmaceutically acceptable salt, stereoisomer, solvate, prodrug, isotopic derivative, or tautomer thereof wherein all variables are as defined herein.

In some embodiments, the compound is of Formula (I-I-I-A-I-a-1-A-1-H):

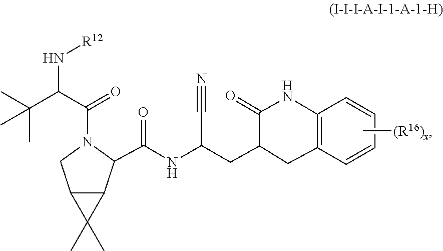

or a pharmaceutically acceptable salt, stereoisomer, solvate, prodrug, isotopic derivative, or tautomer thereof wherein x is an integer selected from 0, 1, 2, 3, 4 and all other variables are as defined herein.

In some embodiments, the compound is of Formula (I-A-I-a-1-A-1-H-a):

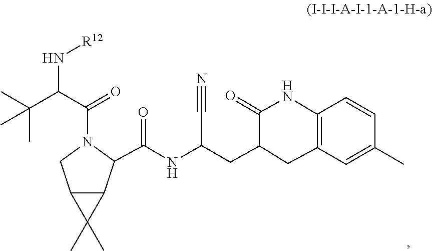

or a pharmaceutically acceptable salt, stereoisomer, solvate, prodrug, isotopic derivative, or tautomer thereof.

In some embodiments, the compound is of Formula (I-I-II):

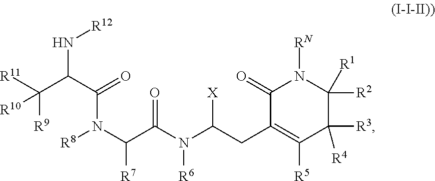

or a pharmaceutically acceptable salt, stereoisomer, solvate, prodrug, isotopic derivative, or tautomer thereof wherein all variables are as defined herein.

In some embodiments, the compound is of Formula (I-I-II-I):

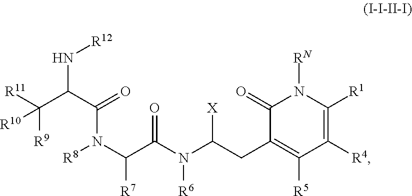

or a pharmaceutically acceptable salt, stereoisomer, solvate, prodrug, isotopic derivative, or tautomer thereof wherein all variables are as defined herein.

In some embodiments, the compound is of Formula (I-I-II-I-a):

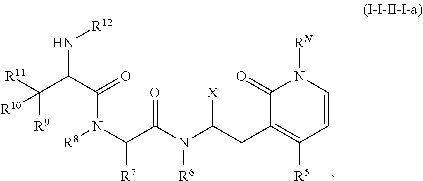

or a pharmaceutically acceptable salt, stereoisomer, solvate, prodrug, isotopic derivative, or tautomer thereof wherein all variables are as defined herein.

In some embodiments, the compound is of Formula (I-I-II-I-a-1):

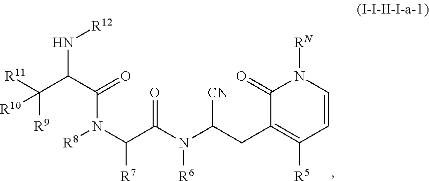

or a pharmaceutically acceptable salt, stereoisomer, solvate, prodrug, isotopic derivative, or tautomer thereof wherein all variables are as defined herein.

In some embodiments, the compound is of Formula (I-I-II-I-a-1-A):

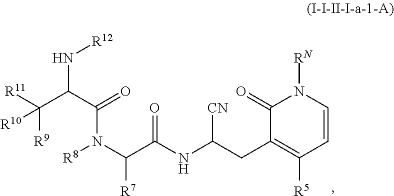

or a pharmaceutically acceptable salt, stereoisomer, solvate, prodrug, isotopic derivative, or tautomer thereof wherein all variables are as defined herein.

In some embodiments, the compound is of Formula (I-I-II-I-a-1-A-I):

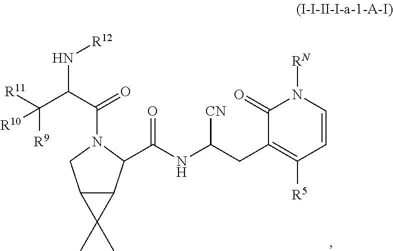

or a pharmaceutically acceptable salt, stereoisomer, solvate, prodrug, isotopic derivative, or tautomer thereof wherein all variables are as defined herein.

In some embodiments, the compound is of Formula (I-I-II-I-a-1-A-I-a):

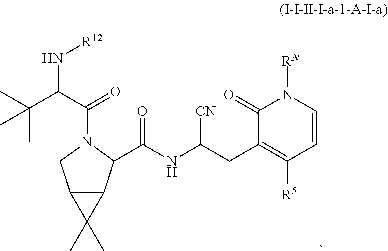

or a pharmaceutically acceptable salt, stereoisomer, solvate, prodrug, isotopic derivative, or tautomer thereof wherein all variables are as defined herein.

In some embodiments, the compound is of Formula (I-I-II-I-a-1-A-I-a*):

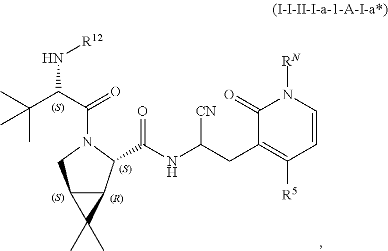

or a pharmaceutically acceptable salt, stereoisomer, solvate, prodrug, isotopic derivative, or tautomer thereof wherein all variables are as defined herein.

In some embodiments, the compound is of Formula (I-I-II-II):

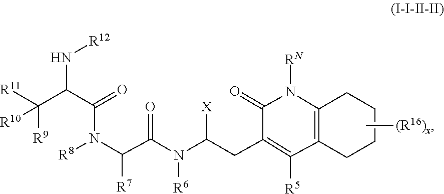

or a pharmaceutically acceptable salt, stereoisomer, solvate, prodrug, isotopic derivative, or tautomer thereof wherein x is an integer selected from 0, 1, 2, 3, 4 and all other variables are as defined herein.

In some embodiments, the compound is of Formula (I-I-II-II-a):

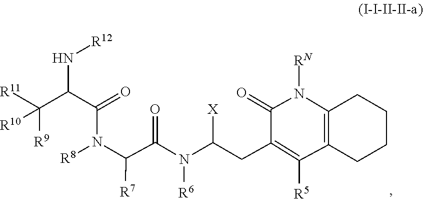

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1):

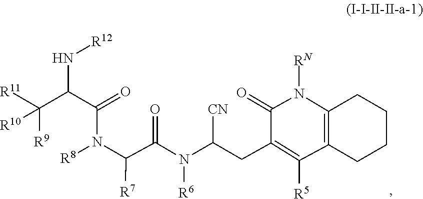

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2):

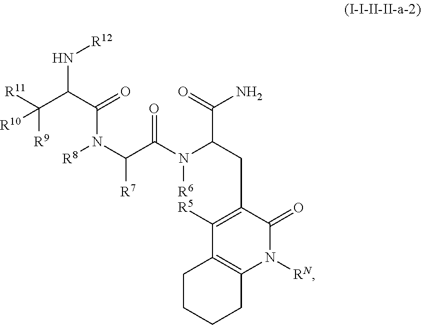

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-A):

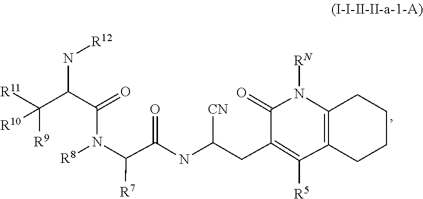

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-B):

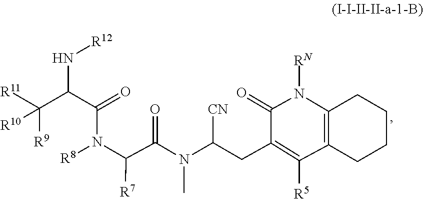

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2-A):

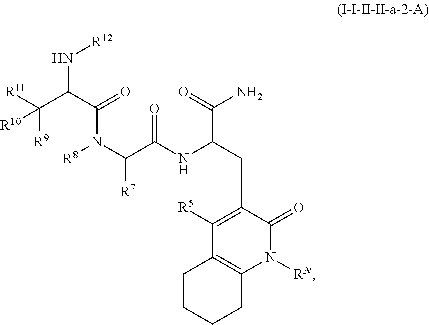

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2-B):

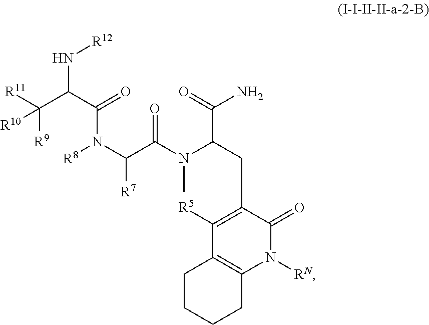

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-A-I):

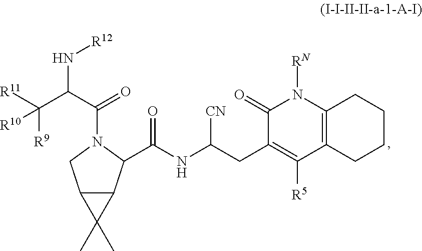

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2-A-I):

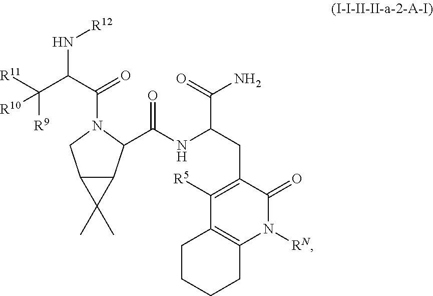

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-B-I):

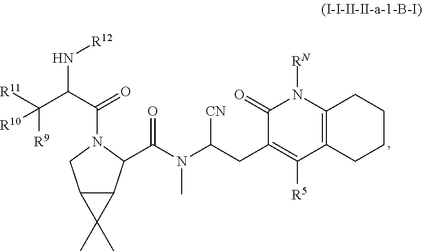

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2-B-I):

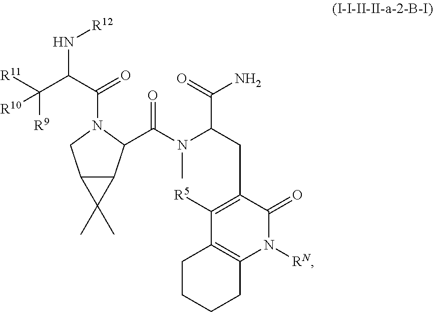

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-A-II):

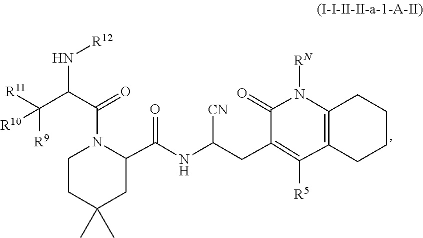

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2-A-II):

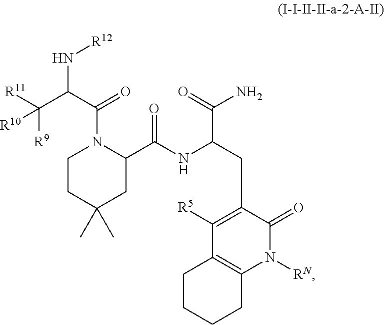

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-B-II):

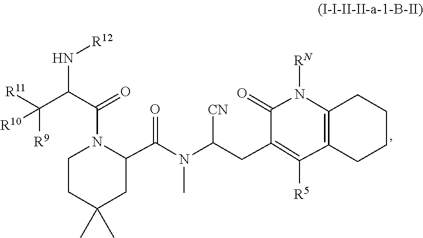

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2-B-II):

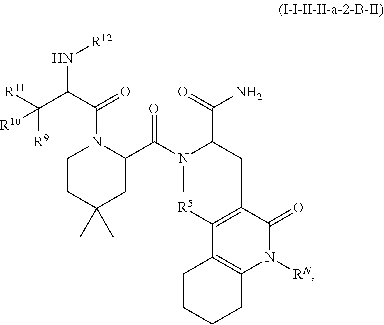

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-A-III):

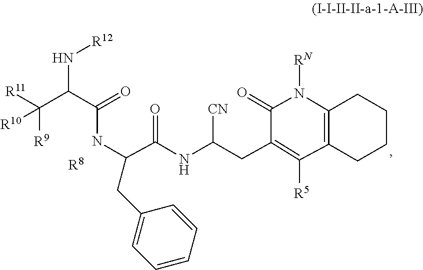

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2-A-III):

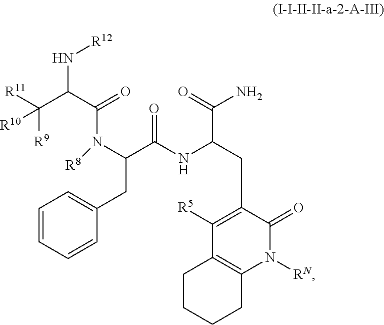

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-B-III):

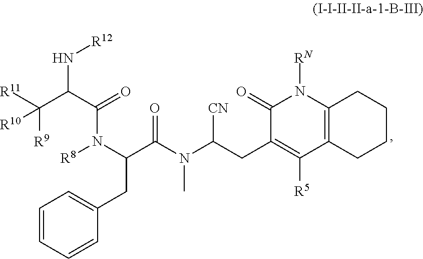

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2-B-III):

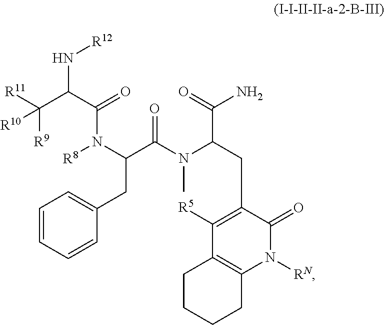

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-A-IV):

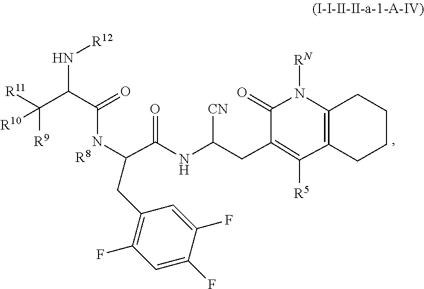

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2-A-IV):

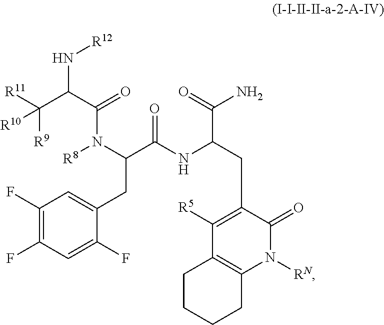

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-B-IV):

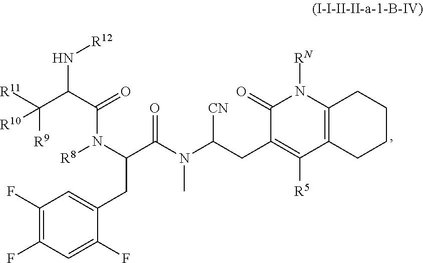

or a pharmaceutically acceptable salt, stereoisomer, solvate, prodrug, isotopic derivative, or tautomer thereof wherein all variables are as defined herein.

In some embodiments, the compound is of Formula (I-I-II-II-a-2-B-IV):

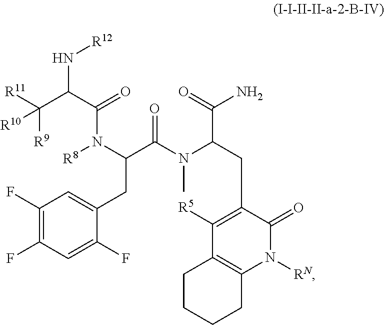

or a pharmaceutically acceptable salt, stereoisomer, solvate, prodrug, isotopic derivative, or tautomer thereof wherein all variables are as defined herein.

In some embodiments, the compound is of Formula (I-I-II-II-a-i-A-I-a):

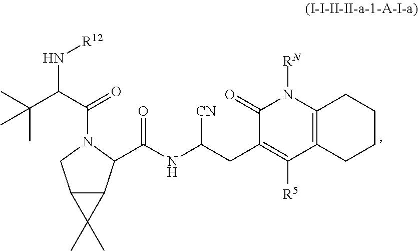

or a pharmaceutically acceptable salt, stereoisomer, solvate, prodrug, isotopic derivative, or tautomer thereof wherein all variables are as defined herein.

In some embodiments, the compound is of Formula (I-I-II-II-a-1-A-I-a*):

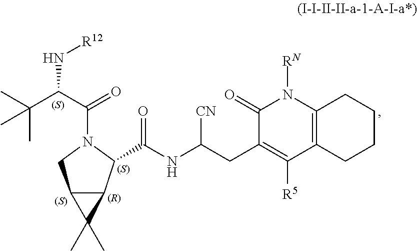

or a pharmaceutically acceptable salt, stereoisomer, solvate, prodrug, isotopic derivative, or tautomer thereof wherein all variables are as defined herein.

In some embodiments, the compound is of Formula (I-I-II-II-b):

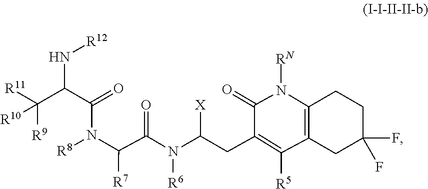

or a pharmaceutically acceptable salt, stereoisomer, solvate, prodrug, isotopic derivative, or tautomer thereof wherein all variables are as defined herein.

In some embodiments, the compound is of Formula (I-I-II-II-b-1):

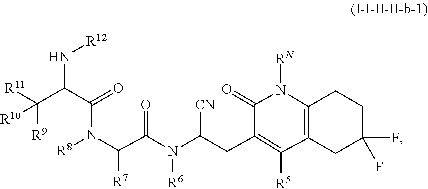

or a pharmaceutically acceptable salt, stereoisomer, solvate, prodrug, isotopic derivative, or tautomer thereof wherein all variables are as defined herein.

In some embodiments, the compound is of Formula (I-I-II-II-b-1-A):

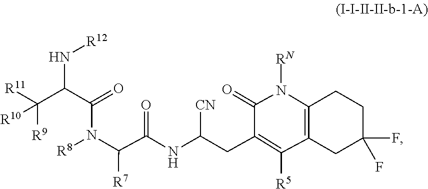

or a pharmaceutically acceptable salt, stereoisomer, solvate, prodrug, isotopic derivative, or tautomer thereof wherein all variables are as defined herein.

In some embodiments, the compound is of Formula (I-I-II-II-b-1-A-I):

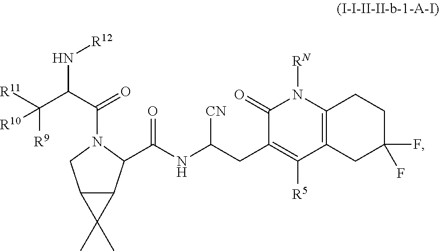

or a pharmaceutically acceptable salt, stereoisomer, solvate, prodrug, isotopic derivative, or tautomer thereof wherein all variables are as defined herein.

In some embodiments, the compound is of Formula (I-I-II-II-b-1-A-I-a):

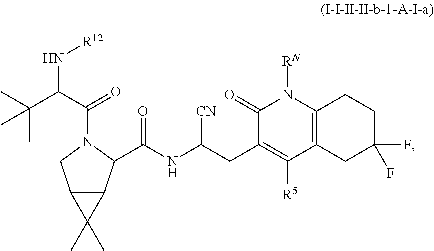

or a pharmaceutically acceptable salt, stereoisomer, solvate, prodrug, isotopic derivative, or tautomer thereof wherein all variables are as defined herein.

In some embodiments, the compound is of Formula (I-I-II-II-b-1-A-I-a*):

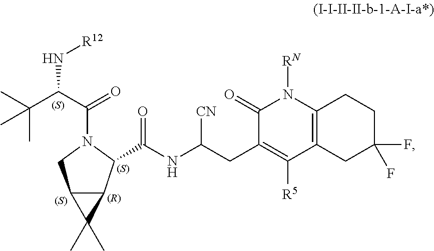

or a pharmaceutically acceptable salt, stereoisomer, solvate, prodrug, isotopic derivative, or tautomer thereof wherein all variables are as defined herein.

In some embodiments, the compound is of Formula (I-I-II-II-c):

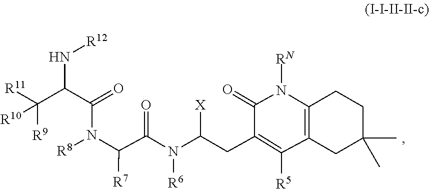

or a pharmaceutically acceptable salt, stereoisomer, solvate, prodrug, isotopic derivative, or tautomer thereof wherein all variables are as defined herein.

In some embodiments, the compound is of Formula (I-I-II-II-c-1):

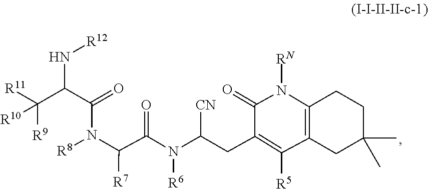

or a pharmaceutically acceptable salt, stereoisomer, solvate, prodrug, isotopic derivative, or tautomer thereof wherein all variables are as defined herein.

In some embodiments, the compound is of Formula (I-I-II-II-c-1-A):

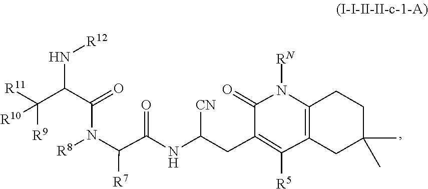

or a pharmaceutically acceptable salt, stereoisomer, solvate, prodrug, isotopic derivative, or tautomer thereof wherein all variables are as defined herein.

In some embodiments, the compound is of Formula (I-I-II-II-c-1-A-I):

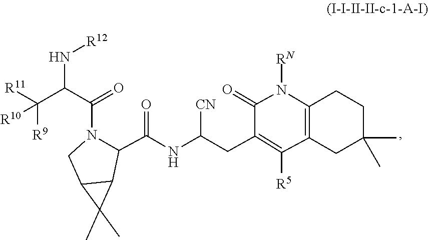

or a pharmaceutically acceptable salt, stereoisomer, solvate, prodrug, isotopic derivative, or tautomer thereof wherein all variables are as defined herein.

In some embodiments, the compound is of Formula (I-I-II-II-c-1-A-I-a):

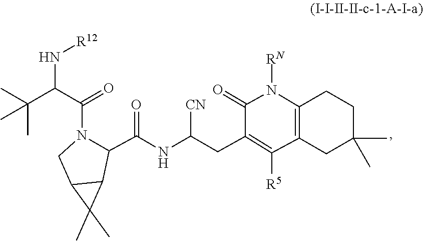

or a pharmaceutically acceptable salt, stereoisomer, solvate, prodrug, isotopic derivative, or tautomer thereof wherein all variables are as defined herein.

In some embodiments, the compound is of Formula (I-I-II-II-c-1-A-I-a*):

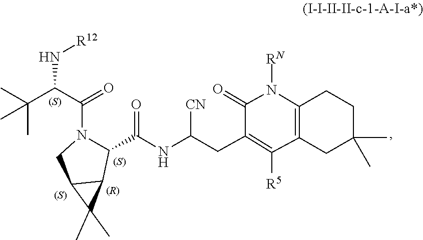

or a pharmaceutically acceptable salt, stereoisomer, solvate, prodrug, isotopic derivative, or tautomer thereof wherein all variables are as defined herein.

In some embodiments, the compound is of Formula (I-I-II-III):

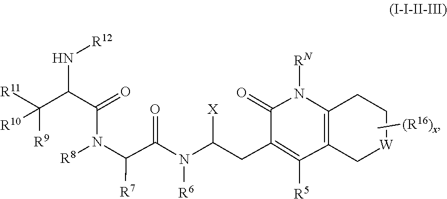

or a pharmaceutically acceptable salt, stereoisomer, solvate, prodrug, isotopic derivative, or tautomer thereof wherein x is an integer selected from 0, 1, 2, 3, 4, 5; W is selected from O, S, S(O), S(O)₂, NR^{16′}; R^{16′} is selected from H, C₁-C₆ alkyl, C₃-C₇ cycloalkyl, —C(O)C₁-C₆ alkyl, and other variables are as defined herein.

In some embodiments, the compound is of Formula (I-I-II-III-a):

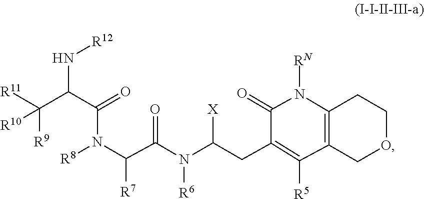

or a pharmaceutically acceptable salt, stereoisomer, solvate, prodrug, isotopic derivative, or tautomer thereof wherein all variables are as defined herein.

In some embodiments, the compound is of Formula (I-I-II-III-a-1):

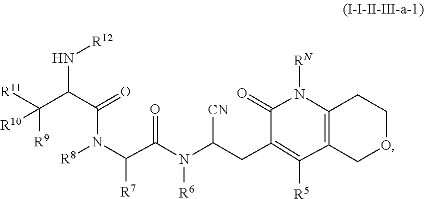

or a pharmaceutically acceptable salt, stereoisomer, solvate, prodrug, isotopic derivative, or tautomer thereof wherein all variables are as defined herein.

In some embodiments, the compound is of Formula (I-I-II-III-a-1-A):

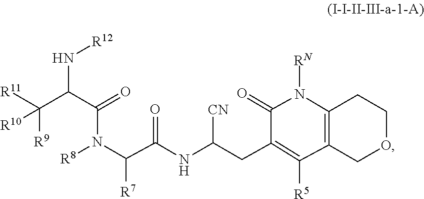

or a pharmaceutically acceptable salt, stereoisomer, solvate, prodrug, isotopic derivative, or tautomer thereof wherein all variables are as defined herein.

In some embodiments, the compound is of Formula (I-I-II-III-a-1-A-I):

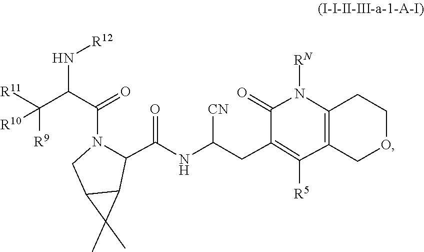

or a pharmaceutically acceptable salt, stereoisomer, solvate, prodrug, isotopic derivative, or tautomer thereof wherein all variables are as defined herein.

In some embodiments, the compound is of Formula (I-I-II-III-a-1-A-I-a):

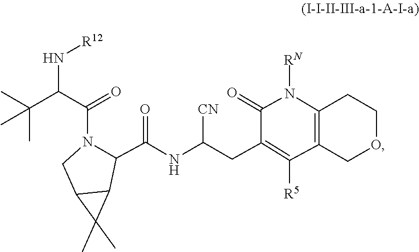

or a pharmaceutically acceptable salt, stereoisomer, solvate, prodrug, isotopic derivative, or tautomer thereof wherein all variables are as defined herein.

In some embodiments, the compound is of Formula (I-I-II-III-a-1-A-I-a*):

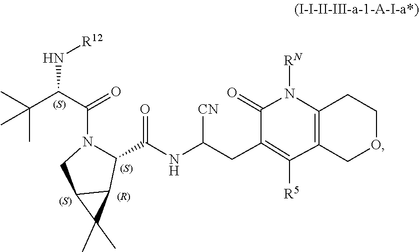

or a pharmaceutically acceptable salt, stereoisomer, solvate, prodrug, isotopic derivative, or tautomer thereof wherein all variables are as defined herein.

In some embodiments, the compound is of Formula (I-I-II-III-b):

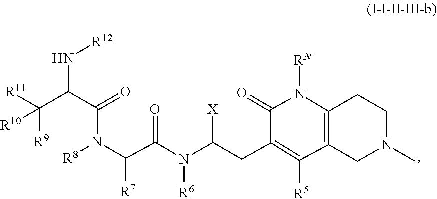

or a pharmaceutically acceptable salt, stereoisomer, solvate, prodrug, isotopic derivative, or tautomer thereof wherein all variables are as defined herein.

In some embodiments, the compound is of Formula (I-I-II-III-b-1):

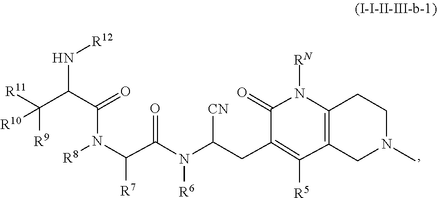

or a pharmaceutically acceptable salt, stereoisomer, solvate, prodrug, isotopic derivative, or tautomer thereof wherein all variables are as defined herein.

In some embodiments, the compound is of Formula (I-I-II-III-b-1-A):

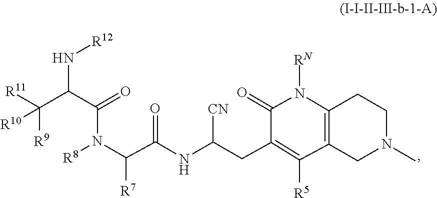

or a pharmaceutically acceptable salt, stereoisomer, solvate, prodrug, isotopic derivative, or tautomer thereof wherein all variables are as defined herein.

In some embodiments, the compound is of Formula (I-I-II-III-b-1-A-I):

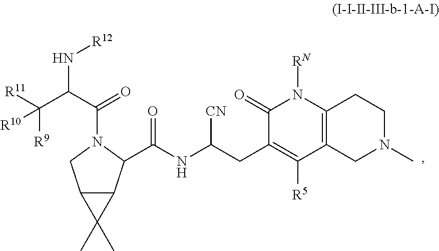

or a pharmaceutically acceptable salt, stereoisomer, solvate, prodrug, isotopic derivative, or tautomer thereof wherein all variables are as defined herein.

In some embodiments, the compound is of Formula (I-I-II-III-b-1-A-I-a):

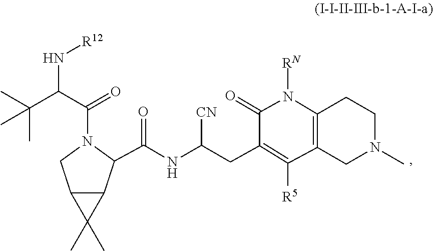

or a pharmaceutically acceptable salt, stereoisomer, solvate, prodrug, isotopic derivative, or tautomer thereof wherein all variables are as defined herein.

In some embodiments, the compound is of Formula (I-I-II-III-b-1-A-I-a*):

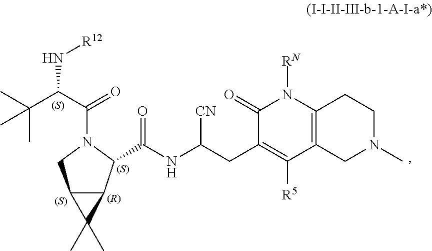

or a pharmaceutically acceptable salt, stereoisomer, solvate, prodrug, isotopic derivative, or tautomer thereof wherein all variables are as defined herein.

In some embodiments, the compound is of Formula (I-I-II-III-c):

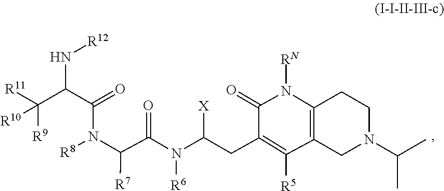

or a pharmaceutically acceptable salt, stereoisomer, solvate, prodrug, isotopic derivative, or tautomer thereof wherein all variables are as defined herein.

In some embodiments, the compound is of Formula (I-I-II-III-c-1):

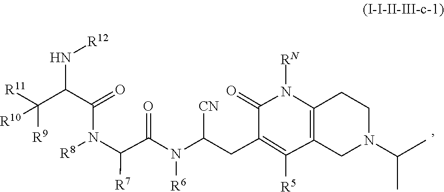

or a pharmaceutically acceptable salt, stereoisomer, solvate, prodrug, isotopic derivative, or tautomer thereof wherein all variables are as defined herein.

In some embodiments, the compound is of Formula (I-I-II-III-c-1-A):

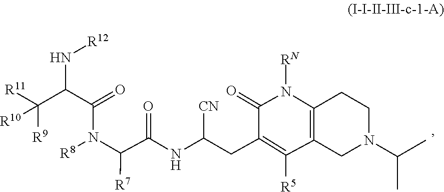

or a pharmaceutically acceptable salt, stereoisomer, solvate, prodrug, isotopic derivative, or tautomer thereof wherein all variables are as defined herein.

In some embodiments, the compound is of Formula (I-I-II-III-c-1-A-I):

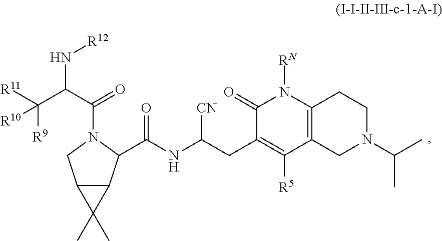

or a pharmaceutically acceptable salt, stereoisomer, solvate, prodrug, isotopic derivative, or tautomer thereof wherein all variables are as defined herein.

In some embodiments, the compound is of Formula (I-I-II-III-c-1-A-I-a):

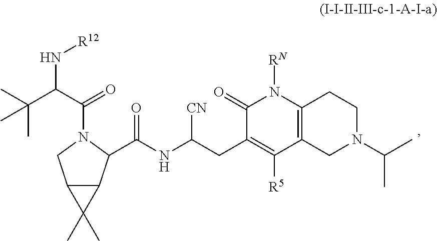

or a pharmaceutically acceptable salt, stereoisomer, solvate, prodrug, isotopic derivative, or tautomer thereof wherein all variables are as defined herein.

In some embodiments, the compound is of Formula (I-I-II-III-c-1-A-I-a*):

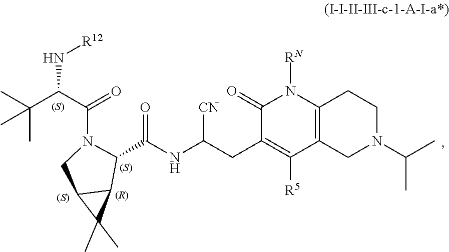

or a pharmaceutically acceptable salt, stereoisomer, solvate, prodrug, isotopic derivative, or tautomer thereof wherein all variables are as defined herein.

In some embodiments, the compound is of Formula (I-I-II-III-d):

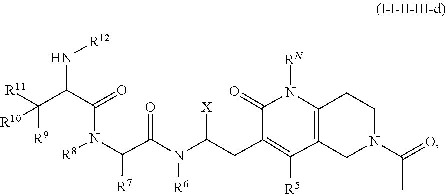

or a pharmaceutically acceptable salt, stereoisomer, solvate, prodrug, isotopic derivative, or tautomer thereof wherein all variables are as defined herein.

In some embodiments, the compound is of Formula (I-I-II-III-d-1):

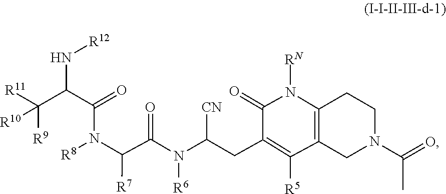

or a pharmaceutically acceptable salt, stereoisomer, solvate, prodrug, isotopic derivative, or tautomer thereof wherein all variables are as defined herein.

In some embodiments, the compound is of Formula (I-I-II-III-d-1-A):

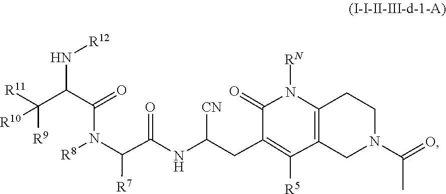

or a pharmaceutically acceptable salt, stereoisomer, solvate, prodrug, isotopic derivative, or tautomer thereof wherein all variables are as defined herein.

In some embodiments, the compound is of Formula (I-I-II-III-d-1-A-I):

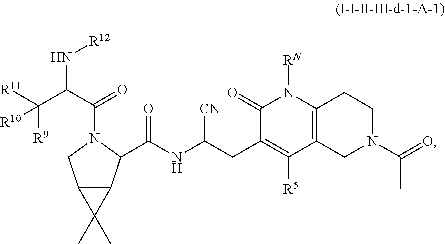

or a pharmaceutically acceptable salt, stereoisomer, solvate, prodrug, isotopic derivative, or tautomer thereof wherein all variables are as defined herein.

In some embodiments, the compound is of Formula (I-I-II-III-d-1-A-I-a):

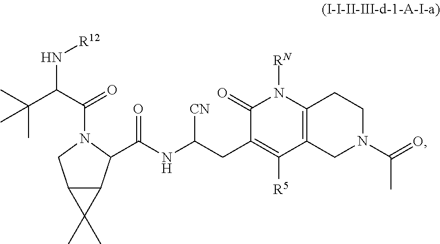

or a pharmaceutically acceptable salt, stereoisomer, solvate, prodrug, isotopic derivative, or tautomer thereof wherein all variables are as defined herein.

In some embodiments, the compound is of Formula (I-I-II-III-d-1-A-I-a*):

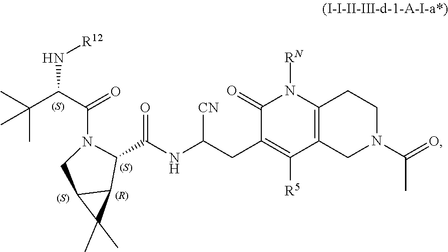

or a pharmaceutically acceptable salt, stereoisomer, solvate, prodrug, isotopic derivative, or tautomer thereof wherein all variables are as defined herein.

In some embodiments, the compound is of Formula (I-I-II-IV):

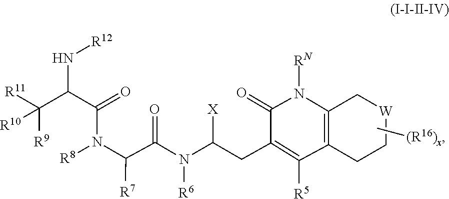

or a pharmaceutically acceptable salt, stereoisomer, solvate, prodrug, isotopic derivative, or tautomer thereof wherein x is an integer selected from 0, 1, 2, 3, 4; W is selected from O, S, S(O), S(O)₂, NR^{16′}; R^{16′} is selected from H, C₁-C₆ alkyl, C₃-C₇ cycloalkyl, —C(O)C₁-C₆ alkyl, and other variables are as defined herein.

In some embodiments, the compound is of Formula (I-I-II-IV-a):

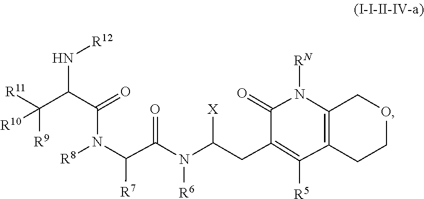

or a pharmaceutically acceptable salt, stereoisomer, solvate, prodrug, isotopic derivative, or tautomer thereof wherein all variables are as defined herein.

In some embodiments, the compound is of Formula (I-I-II-IV-a-1):

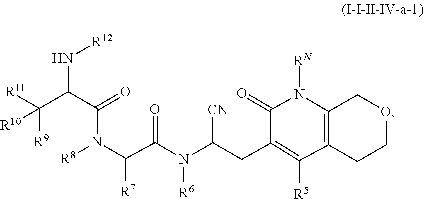

or a pharmaceutically acceptable salt, stereoisomer, solvate, prodrug, isotopic derivative, or tautomer thereof wherein all variables are as defined herein.

In some embodiments, the compound is of Formula (I-I-II-IV-a-1-A):

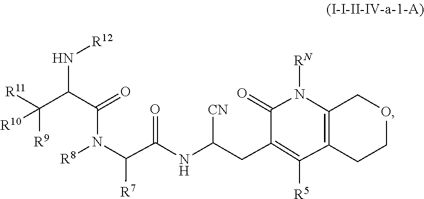

or a pharmaceutically acceptable salt, stereoisomer, solvate, prodrug, isotopic derivative, or tautomer thereof wherein all variables are as defined herein.

In some embodiments, the compound is of Formula (I-I-II-IV-a-1-A-I):

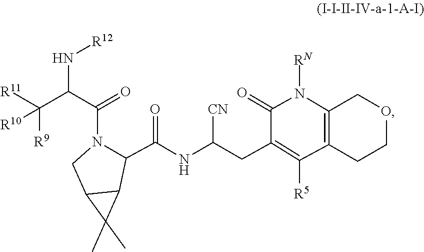

or a pharmaceutically acceptable salt, stereoisomer, solvate, prodrug, isotopic derivative, or tautomer thereof wherein all variables are as defined herein.

In some embodiments, the compound is of Formula (I-I-II-IV-a-1-A-I-a):

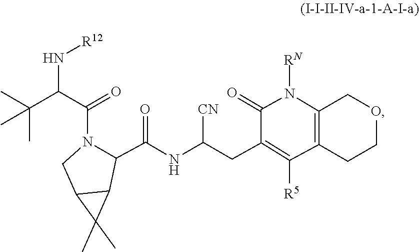

or a pharmaceutically acceptable salt, stereoisomer, solvate, prodrug, isotopic derivative, or tautomer thereof wherein all variables are as defined herein.

In some embodiments, the compound is of Formula (I-I-II-IV-a-1-A-I-a*):

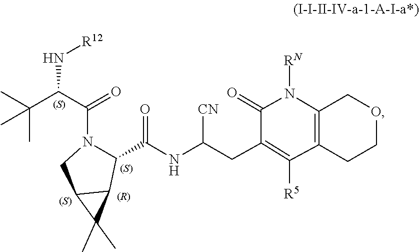

or a pharmaceutically acceptable salt, stereoisomer, solvate, prodrug, isotopic derivative, or tautomer thereof wherein all variables are as defined herein.

In some embodiments, the compound is of Formula (I-I-II-III-b):

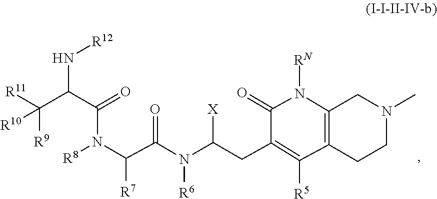

or a pharmaceutically acceptable salt, stereoisomer, solvate, prodrug, isotopic derivative, or tautomer thereof wherein all variables are as defined herein.

In some embodiments, the compound is of Formula (I-I-II-III-b-1):

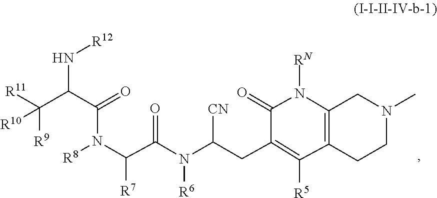

or a pharmaceutically acceptable salt, stereoisomer, solvate, prodrug, isotopic derivative, or tautomer thereof wherein all variables are as defined herein.

In some embodiments, the compound is of Formula (I-I-II-III-b-I-A):

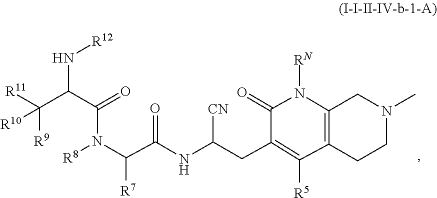

or a pharmaceutically acceptable salt, stereoisomer, solvate, prodrug, isotopic derivative, or tautomer thereof wherein all variables are as defined herein.

In some embodiments, the compound is of Formula (I-I-II-III-b-1-A-I):

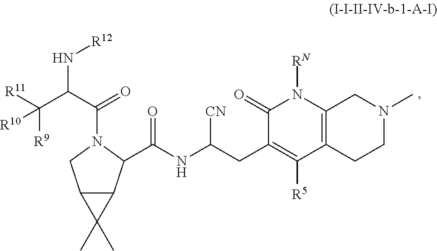

or a pharmaceutically acceptable salt, stereoisomer, solvate, prodrug, isotopic derivative, or tautomer thereof wherein all variables are as defined herein.

In some embodiments, the compound is of Formula (I-I-II-III-b-I-A-I-a):

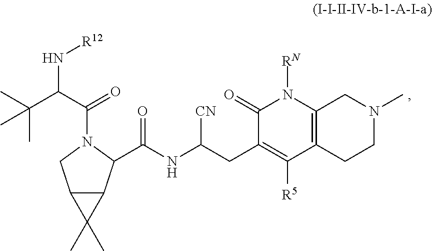

or a pharmaceutically acceptable salt, stereoisomer, solvate, prodrug, isotopic derivative, or tautomer thereof wherein all variables are as defined herein.

In some embodiments, the compound is of Formula (I-I-II-III-b-1-A-I-a*):

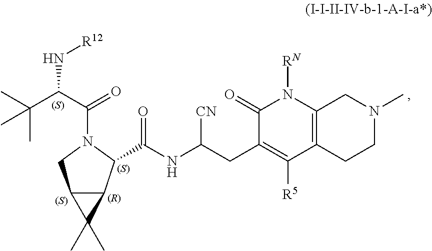

or a pharmaceutically acceptable salt, stereoisomer, solvate, prodrug, isotopic derivative, or tautomer thereof wherein all variables are as defined herein.

In some embodiments, the compound is of Formula (I-I-II-III-c):

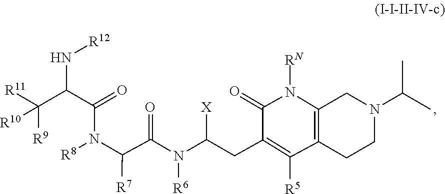

or a pharmaceutically acceptable salt, stereoisomer, solvate, prodrug, isotopic derivative, or tautomer thereof wherein all variables are as defined herein.

In some embodiments, the compound is of Formula (I-I-II-III-c-1):

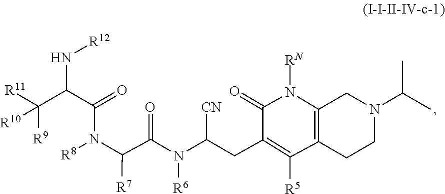

or a pharmaceutically acceptable salt, stereoisomer, solvate, prodrug, isotopic derivative, or tautomer thereof wherein all variables are as defined herein.

In some embodiments, the compound is of Formula (I-I-II-III-c-i-A):

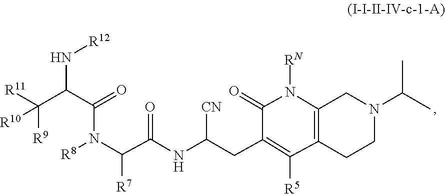

or a pharmaceutically acceptable salt, stereoisomer, solvate, prodrug, isotopic derivative, or tautomer thereof wherein all variables are as defined herein.

In some embodiments, the compound is of Formula (I-I-II-III-c-i-A-I):

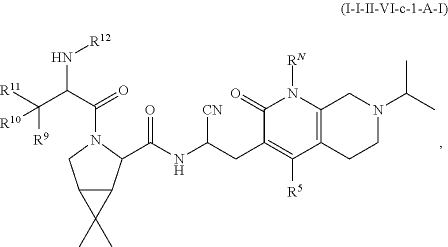

or a pharmaceutically acceptable salt, stereoisomer, solvate, prodrug, isotopic derivative, or tautomer thereof wherein all variables are as defined herein.

In some embodiments, the compound is of Formula (I-I-II-III-c-i-A-I-a):

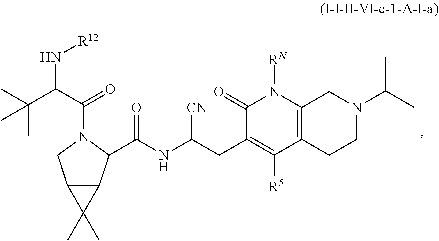

or a pharmaceutically acceptable salt, stereoisomer, solvate, prodrug, isotopic derivative, or tautomer thereof wherein all variables are as defined herein.

In some embodiments, the compound is of Formula (I-I-II-III-c-1-A-I-a*):

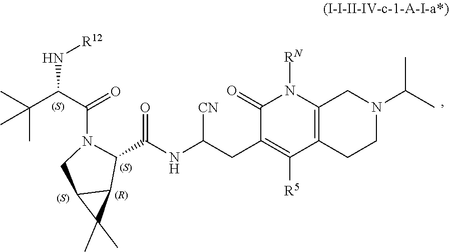

or a pharmaceutically acceptable salt, stereoisomer, solvate, prodrug, isotopic derivative, or tautomer thereof wherein all variables are as defined herein.

In some embodiments, the compound is of Formula (I-I-II-III-d):

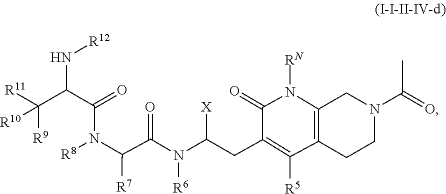

or a pharmaceutically acceptable salt, stereoisomer, solvate, prodrug, isotopic derivative, or tautomer thereof wherein all variables are as defined herein.

In some embodiments, the compound is of Formula (I-I-II-III-d-1):

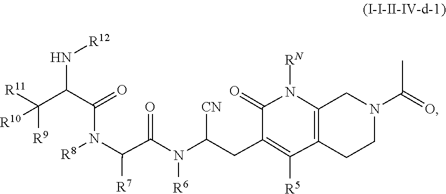

or a pharmaceutically acceptable salt, stereoisomer, solvate, prodrug, isotopic derivative, or tautomer thereof wherein all variables are as defined herein.

In some embodiments, the compound is of Formula (I-I-II-III-d-1-A):

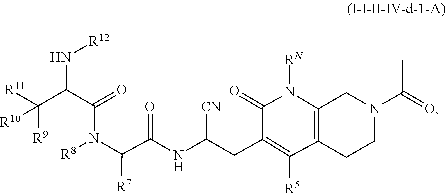

or a pharmaceutically acceptable salt, stereoisomer, solvate, prodrug, isotopic derivative, or tautomer thereof wherein all variables are as defined herein.

In some embodiments, the compound is of Formula (I-I-II-III-d-1-A-I):

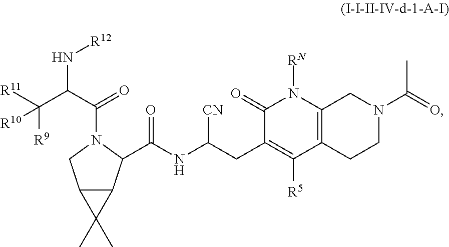

or a pharmaceutically acceptable salt, stereoisomer, solvate, prodrug, isotopic derivative, or tautomer thereof wherein all variables are as defined herein.

In some embodiments, the compound is of Formula (I-I-II-III-d-1-A-I-a):

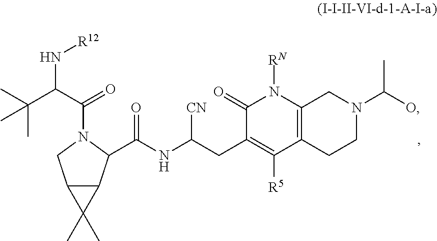

or a pharmaceutically acceptable salt, stereoisomer, solvate, prodrug, isotopic derivative, or tautomer thereof wherein all variables are as defined herein.

In some embodiments, the compound is of Formula (I-I-II-III-d-1-A-I-a*):

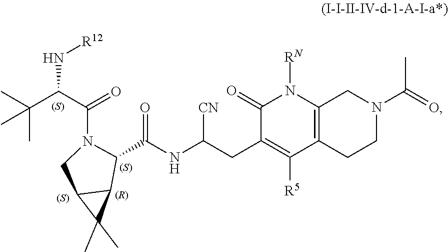

or a pharmaceutically acceptable salt, stereoisomer, solvate, prodrug, isotopic derivative, or tautomer thereof wherein all variables are as defined herein.

In some embodiments, the compound is of Formula (I-I-II-V):

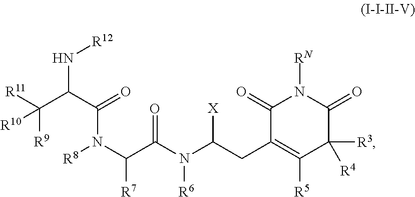

or a pharmaceutically acceptable salt, stereoisomer, solvate, prodrug, isotopic derivative, or tautomer thereof wherein all variables are as defined herein.

In some embodiments, the compound is of Formula (I-I-II-V-a):

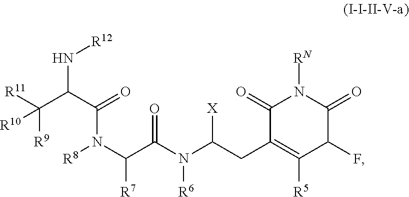

or a pharmaceutically acceptable salt, stereoisomer, solvate, prodrug, isotopic derivative, or tautomer thereof wherein all variables are as defined herein.

In some embodiments, the compound is of Formula (I-I-II-V-a-1):

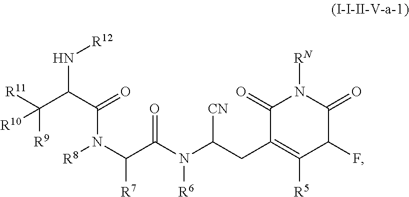

or a pharmaceutically acceptable salt, stereoisomer, solvate, prodrug, isotopic derivative, or tautomer thereof wherein all variables are as defined herein.

In some embodiments, the compound is of Formula (I-I-II-V-a-1-A):

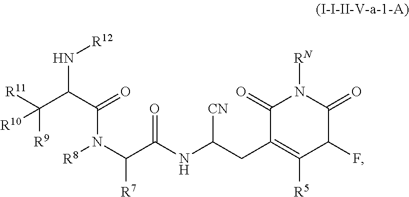

or a pharmaceutically acceptable salt, stereoisomer, solvate, prodrug, isotopic derivative, or tautomer thereof wherein all variables are as defined herein.

In some embodiments, the compound is of Formula (I-I-II-V-a-1-A-I):

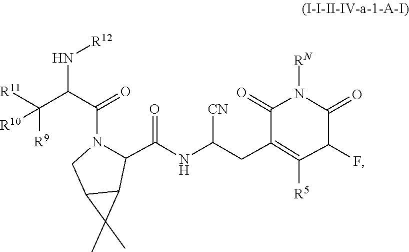

or a pharmaceutically acceptable salt, stereoisomer, solvate, prodrug, isotopic derivative, or tautomer thereof wherein all variables are as defined herein.

In some embodiments, the compound is of Formula (I-I-II-V-a-1-A-I-a):

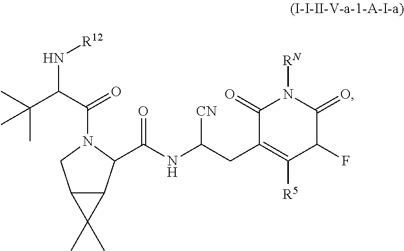

or a pharmaceutically acceptable salt, stereoisomer, solvate, prodrug, isotopic derivative, or tautomer thereof wherein all variables are as defined herein.

In some embodiments, the compound is of Formula (I-I-II-V-a-1-A-I-a*):

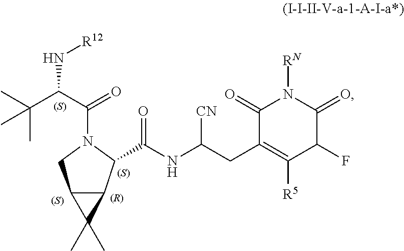

or a pharmaceutically acceptable salt, stereoisomer, solvate, prodrug, isotopic derivative, or tautomer thereof wherein all variables are as defined herein.

In some embodiments, the compound is of Formula (I-I-II-VI):

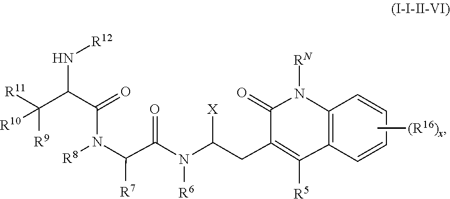

or a pharmaceutically acceptable salt, stereoisomer, solvate, prodrug, isotopic derivative, or tautomer thereof wherein x is an integer selected from 0, 1, 2, 3, 4 and all other variables are as defined herein.

In some embodiments, the compound is of Formula (I-I-II-VI-a):

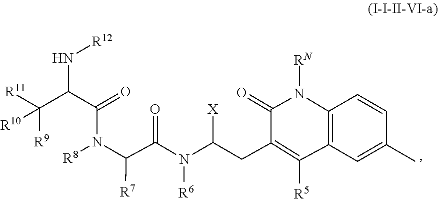

or a pharmaceutically acceptable salt, stereoisomer, solvate, prodrug, isotopic derivative, or tautomer thereof.

In some embodiments, the compound is of Formula (I-I-II-VI-a-1):

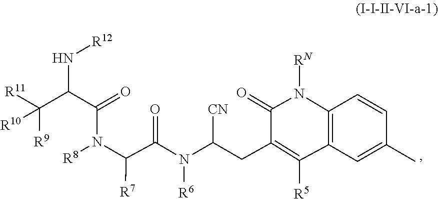

or a pharmaceutically acceptable salt, stereoisomer, solvate, prodrug, isotopic derivative, or tautomer thereof.

In some embodiments, the compound is of Formula (I-I-II-VI-a-1-A):

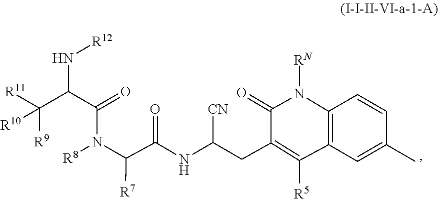

or a pharmaceutically acceptable salt, stereoisomer, solvate, prodrug, isotopic derivative, or tautomer thereof.

In some embodiments, the compound is of Formula (I-I-II-VI-a-1-B):

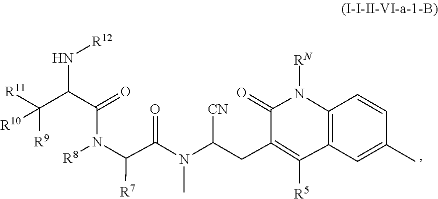

or a pharmaceutically acceptable salt, stereoisomer, solvate, prodrug, isotopic derivative, or tautomer thereof.

In some embodiments, the compound is of Formula (I-I-II-VI-a-1-A-I):

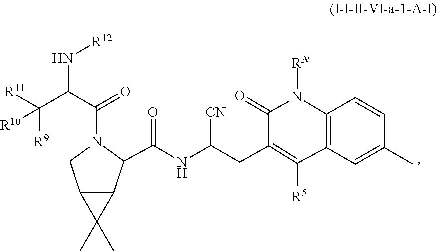

or a pharmaceutically acceptable salt, stereoisomer, solvate, prodrug, isotopic derivative, or tautomer thereof.

In some embodiments, the compound is of Formula (I-I-II-VI-a-1-A-II):

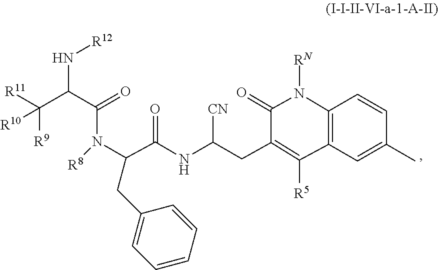

or a pharmaceutically acceptable salt, stereoisomer, solvate, prodrug, isotopic derivative, or tautomer thereof.

In some embodiments, the compound is of Formula (I-I-II-VI-a-1-A-III):

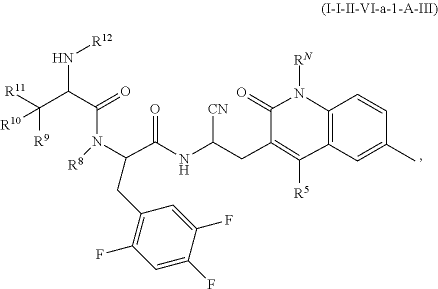

or a pharmaceutically acceptable salt, stereoisomer, solvate, prodrug, isotopic derivative, or tautomer thereof.

In some embodiments, the compound is of Formula (I-I-II-VI-a-1-A-IV):

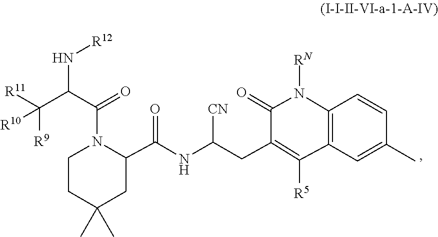

or a pharmaceutically acceptable salt, stereoisomer, solvate, prodrug, isotopic derivative, or tautomer thereof.

In some embodiments, the compound is of Formula (I-I-II-VI-a-1-A-I-a):

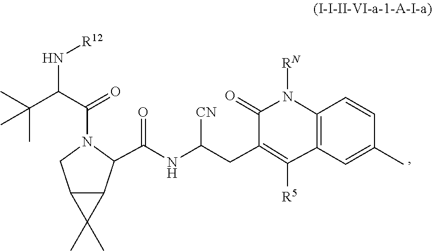

or a pharmaceutically acceptable salt, stereoisomer, solvate, prodrug, isotopic derivative, or tautomer thereof.

In some embodiments, the compound is of Formula (I-I-II-VI-a-1-A-I-a*):

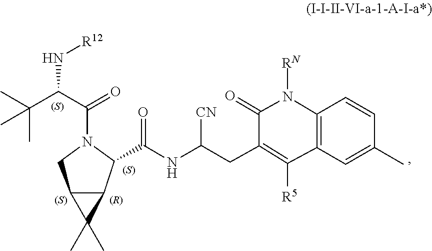

or a pharmaceutically acceptable salt, stereoisomer, solvate, prodrug, isotopic derivative, or tautomer thereof.

In some embodiments, the compound is of Formula (I-I-II-VI-b):

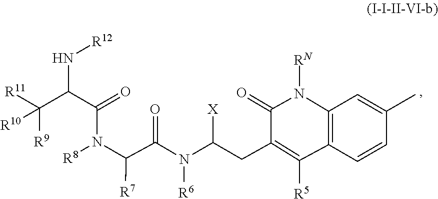

or a pharmaceutically acceptable salt, stereoisomer, solvate, prodrug, isotopic derivative, or tautomer thereof.

In some embodiments, the compound is of Formula (I-I-II-VI-b-1):

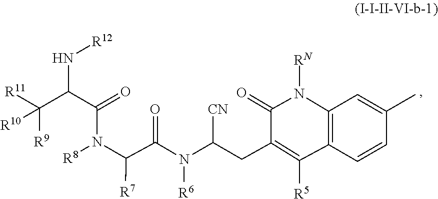

or a pharmaceutically acceptable salt, stereoisomer, solvate, prodrug, isotopic derivative, or tautomer thereof.

In some embodiments, the compound is of Formula (I-I-II-VI-b-1-A):

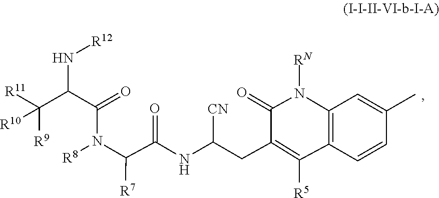

or a pharmaceutically acceptable salt, stereoisomer, solvate, prodrug, isotopic derivative, or tautomer thereof.

In some embodiments, the compound is of Formula (I-I-II-VI-b-1-A-I):

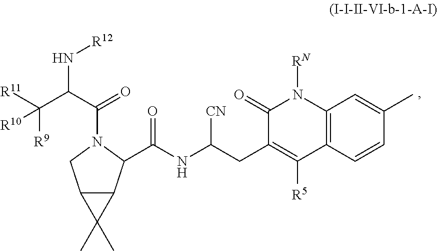

or a pharmaceutically acceptable salt, stereoisomer, solvate, prodrug, isotopic derivative, or tautomer thereof.

In some embodiments, the compound is of Formula (I-I-II-VI-b-1-A-I-a):

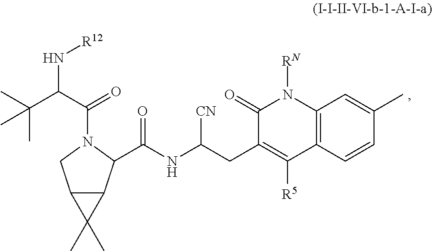

or a pharmaceutically acceptable salt, stereoisomer, solvate, prodrug, isotopic derivative, or tautomer thereof.

In some embodiments, the compound is of Formula (I-I-II-VI-b-1-A-I-a*):

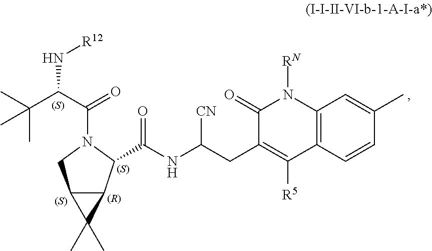

or a pharmaceutically acceptable salt, stereoisomer, solvate, prodrug, isotopic derivative, or tautomer thereof.

In some embodiments, the compound is of Formula (I-I-II-VI-c):

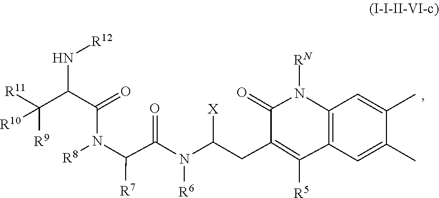

or a pharmaceutically acceptable salt, stereoisomer, solvate, prodrug, isotopic derivative, or tautomer thereof.

In some embodiments, the compound is of Formula (I-I-II-VI-c-1):

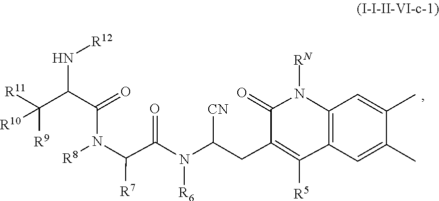

or a pharmaceutically acceptable salt, stereoisomer, solvate, prodrug, isotopic derivative, or tautomer thereof.

In some embodiments, the compound is of Formula (I-I-II-VI-c-1-A):

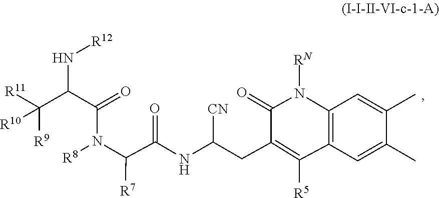

or a pharmaceutically acceptable salt, stereoisomer, solvate, prodrug, isotopic derivative, or tautomer thereof.

In some embodiments, the compound is of Formula (I-I-II-VI-c-1-A-I):

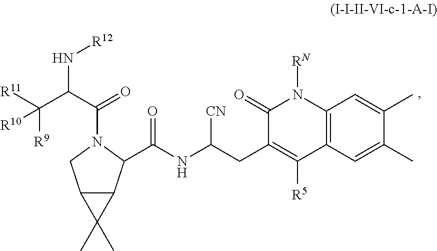

or a pharmaceutically acceptable salt, stereoisomer, solvate, prodrug, isotopic derivative, or tautomer thereof.

In some embodiments, the compound is of Formula (I-I-II-VI-c-1-A-I-a):

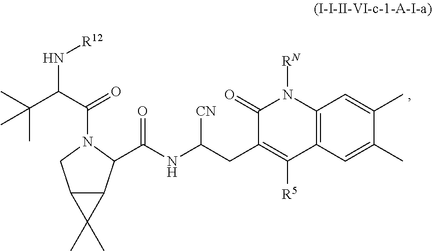

or a pharmaceutically acceptable salt, stereoisomer, solvate, prodrug, isotopic derivative, or tautomer thereof.

In some embodiments, the compound is of Formula (I-I-II-VI-c-1-A-I-a*):

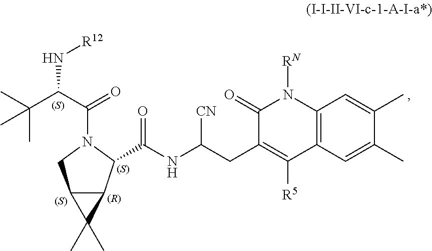

or a pharmaceutically acceptable salt, stereoisomer, solvate, prodrug, isotopic derivative, or tautomer thereof.

In some embodiments, the compound is of Formula (I-I-II-VI-d):

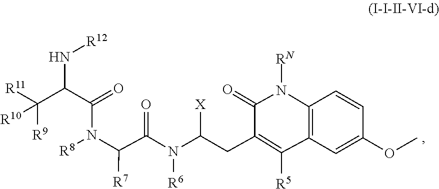

or a pharmaceutically acceptable salt, stereoisomer, solvate, prodrug, isotopic derivative, or tautomer thereof.

In some embodiments, the compound is of Formula (I-I-II-VI-d-1):

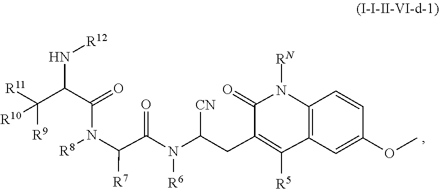

or a pharmaceutically acceptable salt, stereoisomer, solvate, prodrug, isotopic derivative, or tautomer thereof.

In some embodiments, the compound is of Formula (I-I-II-VI-d-1-A):

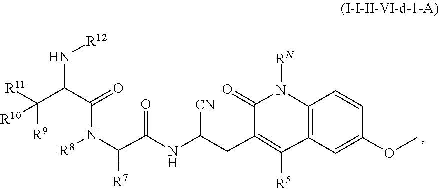

or a pharmaceutically acceptable salt, stereoisomer, solvate, prodrug, isotopic derivative, or tautomer thereof.

In some embodiments, the compound is of Formula (I-I-II-VI-d-1-A-I):

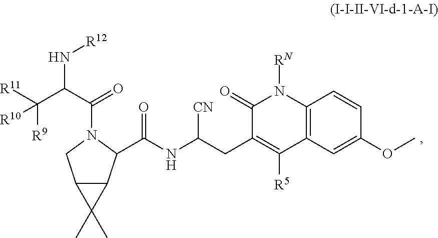

or a pharmaceutically acceptable salt, stereoisomer, solvate, prodrug, isotopic derivative, or tautomer thereof.

In some embodiments, the compound is of Formula (I-I-II-VI-d-1-A-I-a):

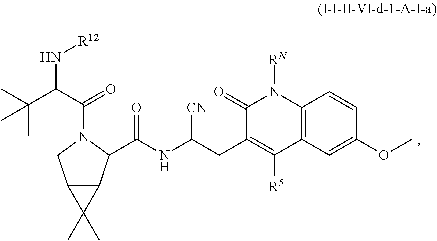

or a pharmaceutically acceptable salt, stereoisomer, solvate, prodrug, isotopic derivative, or tautomer thereof.

In some embodiments, the compound is of Formula (I-I-II-VI-d-1-A-I-a*):

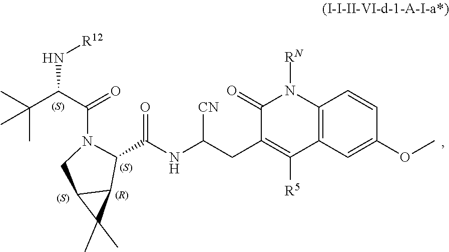

or a pharmaceutically acceptable salt, stereoisomer, solvate, prodrug, isotopic derivative, or tautomer thereof.

In some embodiments, the compound is of Formula (I-I-II-VI-e):

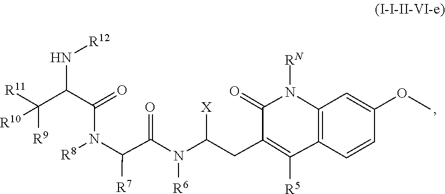

or a pharmaceutically acceptable salt, stereoisomer, solvate, prodrug, isotopic derivative, or tautomer thereof.

In some embodiments, the compound is of Formula (I-I-II-VI-e-1):

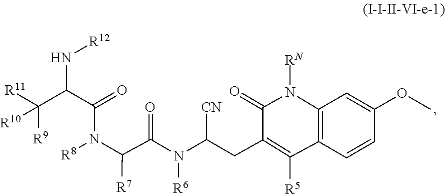

or a pharmaceutically acceptable salt, stereoisomer, solvate, prodrug, isotopic derivative, or tautomer thereof.

In some embodiments, the compound is of Formula (I-I-II-VI-e-1-A):

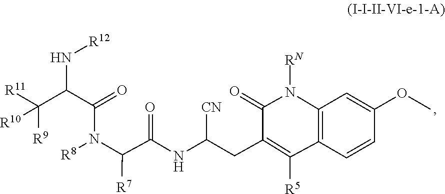

or a pharmaceutically acceptable salt, stereoisomer, solvate, prodrug, isotopic derivative, or tautomer thereof.

In some embodiments, the compound is of Formula (I-I-II-VI-e-1-A-I):

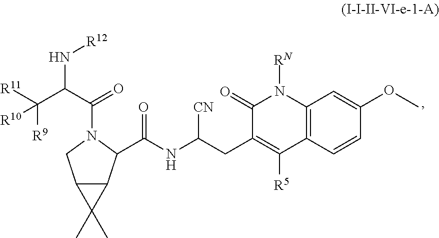

or a pharmaceutically acceptable salt, stereoisomer, solvate, prodrug, isotopic derivative, or tautomer thereof.

In some embodiments, the compound is of Formula (I-I-II-VI-e-1-A-I-a):

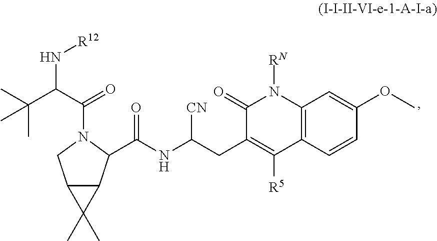

or a pharmaceutically acceptable salt, stereoisomer, solvate, prodrug, isotopic derivative, or tautomer thereof.

In some embodiments, the compound is of Formula (I-I-II-VI-e-1-A-I-a*):

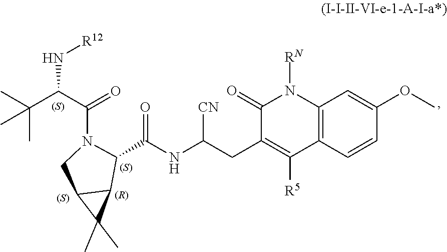

or a pharmaceutically acceptable salt, stereoisomer, solvate, prodrug, isotopic derivative, or tautomer thereof.

In some embodiments, the compound is of Formula (I-I-II-VI-f):

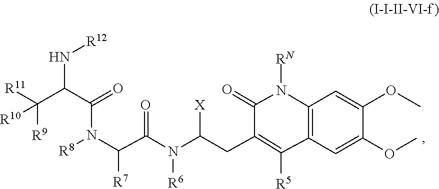

or a pharmaceutically acceptable salt, stereoisomer, solvate, prodrug, isotopic derivative, or tautomer thereof.

In some embodiments, the compound is of Formula (I-I-II-VI-f-1):

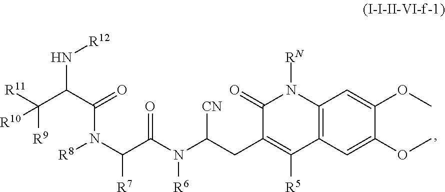

or a pharmaceutically acceptable salt, stereoisomer, solvate, prodrug, isotopic derivative, or tautomer thereof.

In some embodiments, the compound is of Formula (I-I-II-VI-f-1-A):

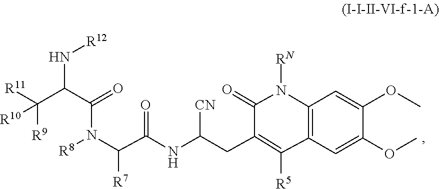

or a pharmaceutically acceptable salt, stereoisomer, solvate, prodrug, isotopic derivative, or tautomer thereof.

In some embodiments, the compound is of Formula (I-I-II-VI-f-1-A-I):

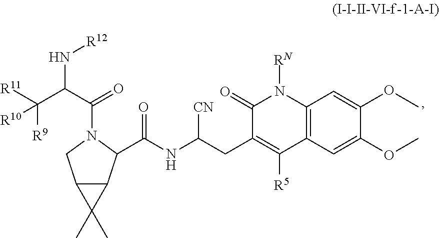

or a pharmaceutically acceptable salt, stereoisomer, solvate, prodrug, isotopic derivative, or tautomer thereof.

In some embodiments, the compound is of Formula (I-I-II-VI-f-1-A-I-a):

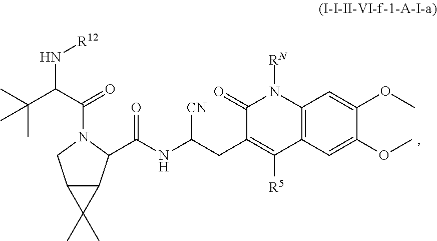

or a pharmaceutically acceptable salt, stereoisomer, solvate, prodrug, isotopic derivative, or tautomer thereof.

In some embodiments, the compound is of Formula (I-I-II-VI-f-1-A-I-a*):

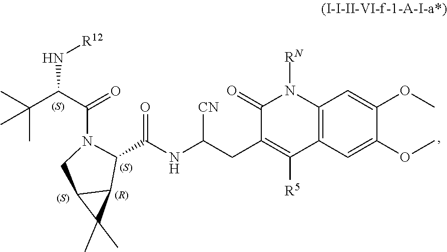

or a pharmaceutically acceptable salt, stereoisomer, solvate, prodrug, isotopic derivative, or tautomer thereof.

In some embodiments, the compound is of Formula (I-I-II-VI-g):

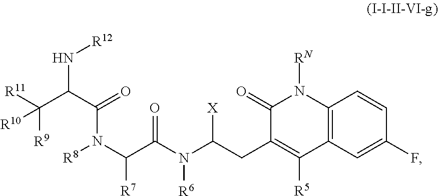

or a pharmaceutically acceptable salt, stereoisomer, solvate, prodrug, isotopic derivative, or tautomer thereof.

In some embodiments, the compound is of Formula (I-I-II-VI-g-1):

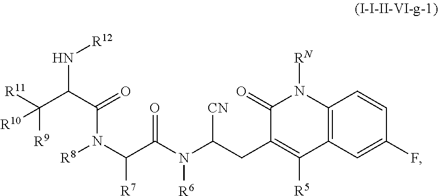

or a pharmaceutically acceptable salt, stereoisomer, solvate, prodrug, isotopic derivative, or tautomer thereof.

In some embodiments, the compound is of Formula (I-I-II-VI-g-1-A):

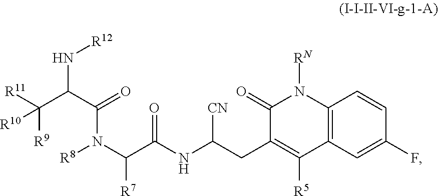

or a pharmaceutically acceptable salt, stereoisomer, solvate, prodrug, isotopic derivative, or tautomer thereof.

In some embodiments, the compound is of Formula (I-I-II-VI-g-1-A-I):

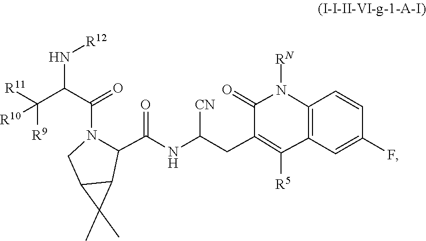

or a pharmaceutically acceptable salt, stereoisomer, solvate, prodrug, isotopic derivative, or tautomer thereof.

In some embodiments, the compound is of Formula (I-I-II-VI-g-1-A-I-a):

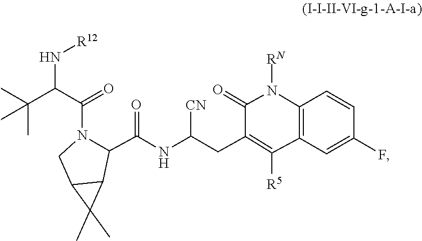

or a pharmaceutically acceptable salt, stereoisomer, solvate, prodrug, isotopic derivative, or tautomer thereof.

In some embodiments, the compound is of Formula (I-I-II-VI-g-1-A-I-a*):

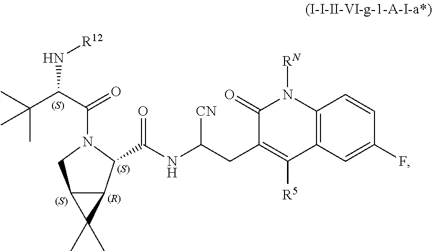

or a pharmaceutically acceptable salt, stereoisomer, solvate, prodrug, isotopic derivative, or tautomer thereof.

In some embodiments, the compound is of Formula (I-I-II-VI-h):

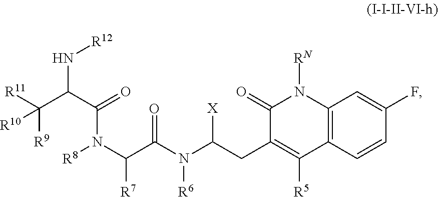

or a pharmaceutically acceptable salt, stereoisomer, solvate, prodrug, isotopic derivative, or tautomer thereof.

In some embodiments, the compound is of Formula (I-I-II-VI-h-1):

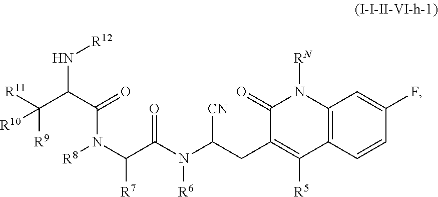

or a pharmaceutically acceptable salt, stereoisomer, solvate, prodrug, isotopic derivative, or tautomer thereof.

In some embodiments, the compound is of Formula (I-I-II-VI-h-1-A):

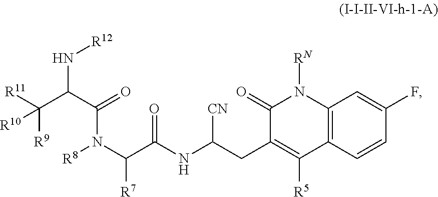

or a pharmaceutically acceptable salt, stereoisomer, solvate, prodrug, isotopic derivative, or tautomer thereof.

In some embodiments, the compound is of Formula (I-I-II-VI-h-1-A-I):

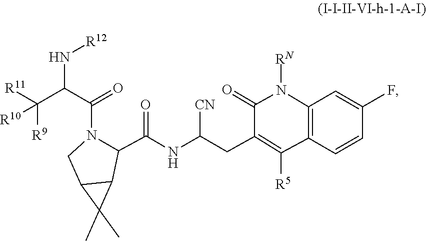

or a pharmaceutically acceptable salt, stereoisomer, solvate, prodrug, isotopic derivative, or tautomer thereof.

In some embodiments, the compound is of Formula (I-I-II-VI-h-1-A-I-a):

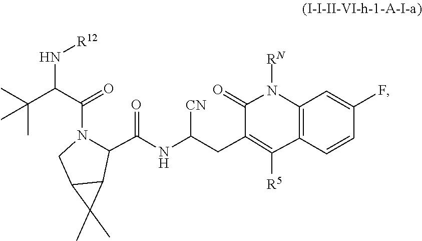

or a pharmaceutically acceptable salt, stereoisomer, solvate, prodrug, isotopic derivative, or tautomer thereof.

In some embodiments, the compound is of Formula (I-I-II-VI-h-1-A-I-a*):

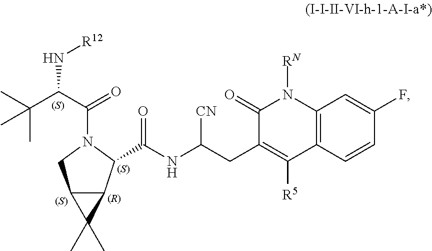

or a pharmaceutically acceptable salt, stereoisomer, solvate, prodrug, isotopic derivative, or tautomer thereof.

In some embodiments, the compound is of Formula (I-I-II-VI-i):

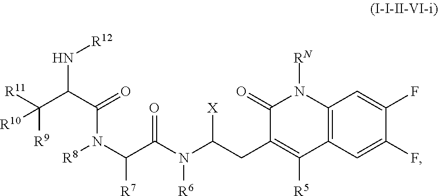

or a pharmaceutically acceptable salt, stereoisomer, solvate, prodrug, isotopic derivative, or tautomer thereof.

In some embodiments, the compound is of Formula (I-I-II-VI-i-1):

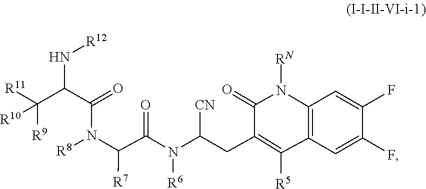

or a pharmaceutically acceptable salt, stereoisomer, solvate, prodrug, isotopic derivative, or tautomer thereof.

In some embodiments, the compound is of Formula (I-I-II-VI-i-1-A):

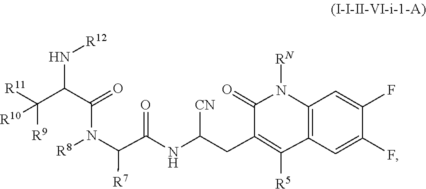

or a pharmaceutically acceptable salt, stereoisomer, solvate, prodrug, isotopic derivative, or tautomer thereof.

In some embodiments, the compound is of Formula (I-I-II-VI-i-1-A-I):

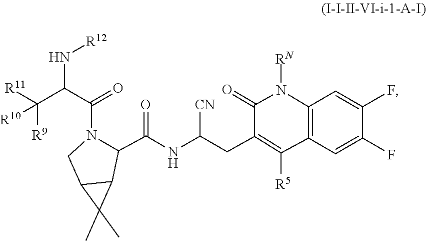

or a pharmaceutically acceptable salt, stereoisomer, solvate, prodrug, isotopic derivative, or tautomer thereof.

In some embodiments, the compound is of Formula (I-I-II-VI-i-1-A-I-a):

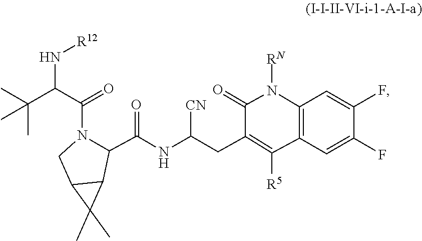

or a pharmaceutically acceptable salt, stereoisomer, solvate, prodrug, isotopic derivative, or tautomer thereof.

In some embodiments, the compound is of Formula (I-I-II-VI-i-1-A-I-a*):

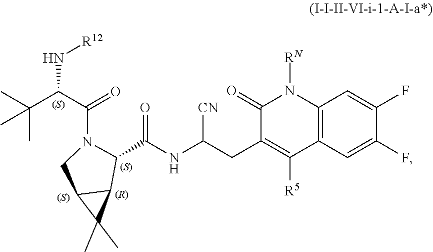

or a pharmaceutically acceptable salt, stereoisomer, solvate, prodrug, isotopic derivative, or tautomer thereof.

In some embodiments, the compound is of Formula (I-I-II-VI-j):

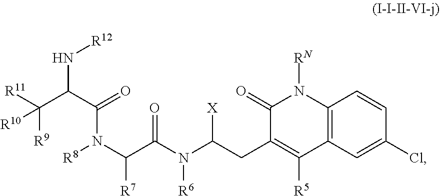

or a pharmaceutically acceptable salt, stereoisomer, solvate, prodrug, isotopic derivative, or tautomer thereof.

In some embodiments, the compound is of Formula (I-I-II-VI-j-1):

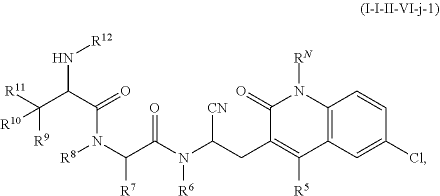

or a pharmaceutically acceptable salt, stereoisomer, solvate, prodrug, isotopic derivative, or tautomer thereof.

In some embodiments, the compound is of Formula (I-I-II-VI-j-1-A):

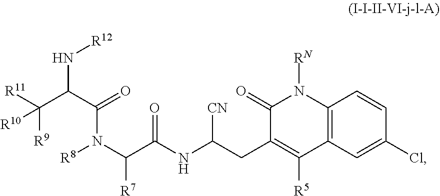

or a pharmaceutically acceptable salt, stereoisomer, solvate, prodrug, isotopic derivative, or tautomer thereof.

In some embodiments, the compound is of Formula (I-I-II-VI-j-1-A-I):

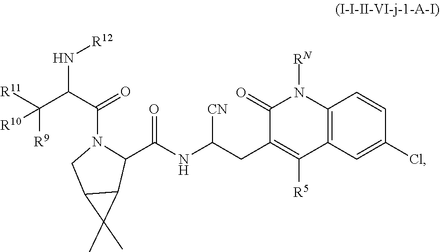

or a pharmaceutically acceptable salt, stereoisomer, solvate, prodrug, isotopic derivative, or tautomer thereof.

In some embodiments, the compound is of Formula (I-I-II-VI-j-1-A-I-a):

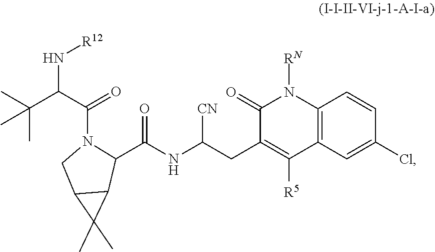

or a pharmaceutically acceptable salt, stereoisomer, solvate, prodrug, isotopic derivative, or tautomer thereof.

In some embodiments, the compound is of Formula (I-I-II-VI-j-1-A-I-a*):

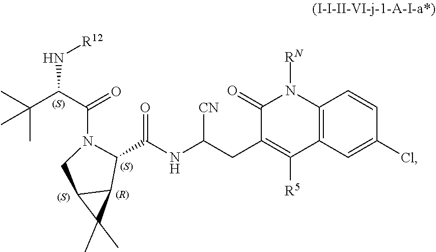

or a pharmaceutically acceptable salt, stereoisomer, solvate, prodrug, isotopic derivative, or tautomer thereof.

In some embodiments, the compound is of Formula (I-I-II-VI-k):

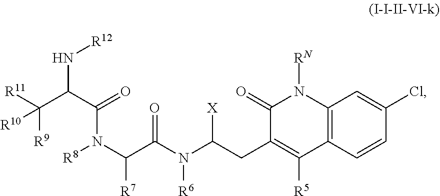

or a pharmaceutically acceptable salt, stereoisomer, solvate, prodrug, isotopic derivative, or tautomer thereof.

In some embodiments, the compound is of Formula (I-I-II-VI-k-1):

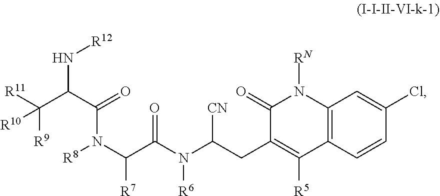

or a pharmaceutically acceptable salt, stereoisomer, solvate, prodrug, isotopic derivative, or tautomer thereof.

In some embodiments, the compound is of Formula (I-I-II-VI-k-1-A):

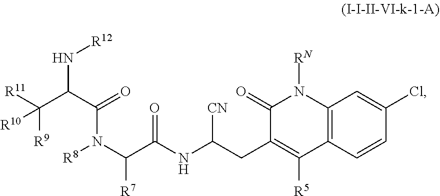

or a pharmaceutically acceptable salt, stereoisomer, solvate, prodrug, isotopic derivative, or tautomer thereof.

In some embodiments, the compound is of Formula (I-I-II-VI-k-1-A-I):

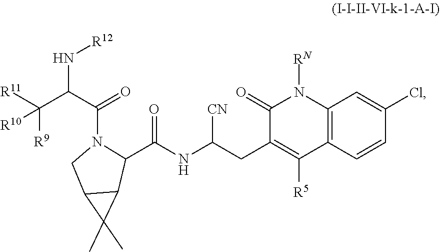

or a pharmaceutically acceptable salt, stereoisomer, solvate, prodrug, isotopic derivative, or tautomer thereof.

In some embodiments, the compound is of Formula (I-I-II-VI-k-1-A-I-a):

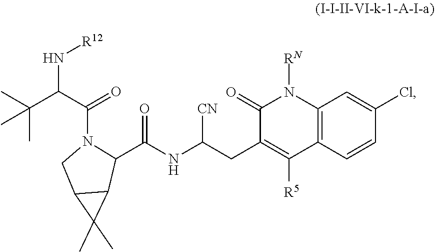

or a pharmaceutically acceptable salt, stereoisomer, solvate, prodrug, isotopic derivative, or tautomer thereof.

In some embodiments, the compound is of Formula (I-I-II-VI-k-1-A-I-a*):

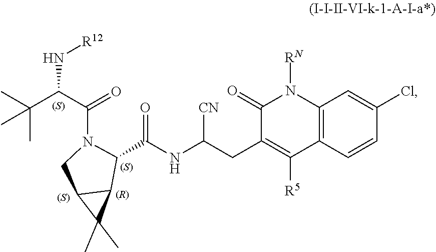

or a pharmaceutically acceptable salt, stereoisomer, solvate, prodrug, isotopic derivative, or tautomer thereof.

In some embodiments, the compound is of Formula (I-I-II-VI-1):

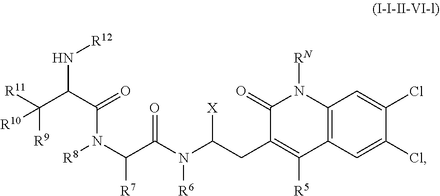

or a pharmaceutically acceptable salt, stereoisomer, solvate, prodrug, isotopic derivative, or tautomer thereof.

In some embodiments, the compound is of Formula (I-I-II-VI-1-1):

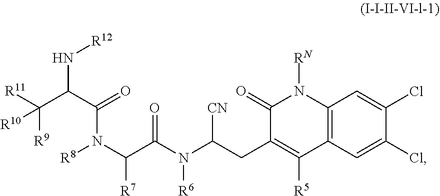

or a pharmaceutically acceptable salt, stereoisomer, solvate, prodrug, isotopic derivative, or tautomer thereof.

In some embodiments, the compound is of Formula (I-I-II-VI-1-1-A):

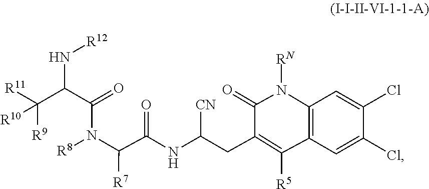

or a pharmaceutically acceptable salt, stereoisomer, solvate, prodrug, isotopic derivative, or tautomer thereof.

In some embodiments, the compound is of Formula (I-I-II-VI-1-1-A-I):

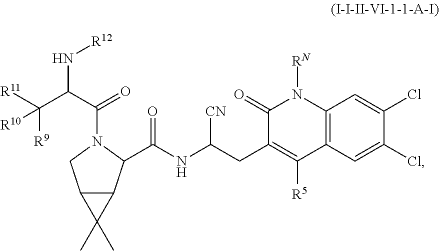

or a pharmaceutically acceptable salt, stereoisomer, solvate, prodrug, isotopic derivative, or tautomer thereof.

In some embodiments, the compound is of Formula (I-I-II-VI-1-1-A-I-a):

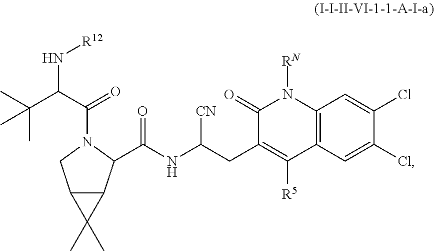

or a pharmaceutically acceptable salt, stereoisomer, solvate, prodrug, isotopic derivative, or tautomer thereof.

In some embodiments, the compound is of Formula (I-I-II-VI-1-1-A-I-a*):

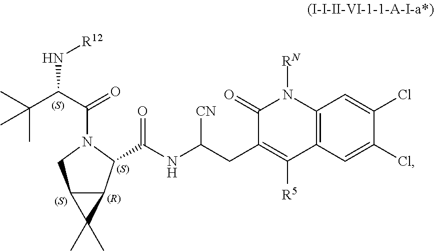

or a pharmaceutically acceptable salt, stereoisomer, solvate, prodrug, isotopic derivative, or tautomer thereof.

In some embodiments, the compound is of Formula (I-I-II-VII):

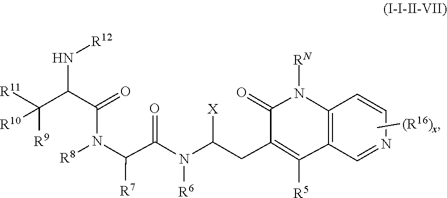

or a pharmaceutically acceptable salt, stereoisomer, solvate, prodrug, isotopic derivative, or tautomer thereof wherein x is an integer selected from 0, 1, 2, 3 and other variables are as defined herein.

In some embodiments, the compound is of Formula (I-I-II-VII-a):

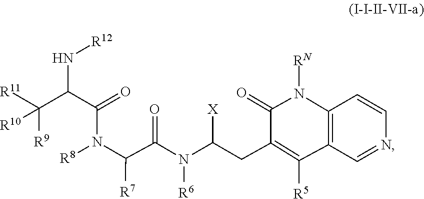

or a pharmaceutically acceptable salt, stereoisomer, solvate, prodrug, isotopic derivative, or tautomer thereof.

In some embodiments, the compound is of Formula (I-I-II-VII-a-1):

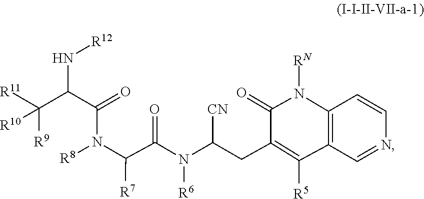

or a pharmaceutically acceptable salt, stereoisomer, solvate, prodrug, isotopic derivative, or tautomer thereof.

In some embodiments, the compound is of Formula (I-I-II-VII-a-1-A):

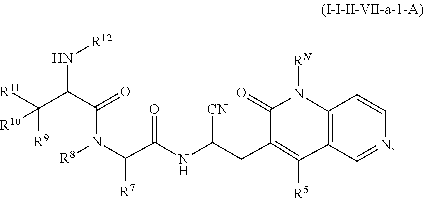

or a pharmaceutically acceptable salt, stereoisomer, solvate, prodrug, isotopic derivative, or tautomer thereof.

In some embodiments, the compound is of Formula (I-I-II-VII-a-1-A-I):

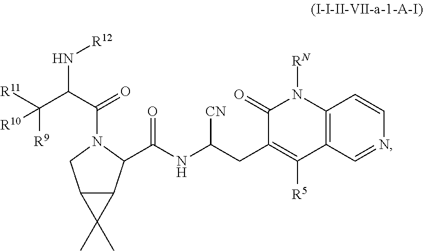

or a pharmaceutically acceptable salt, stereoisomer, solvate, prodrug, isotopic derivative, or tautomer thereof.

In some embodiments, the compound is of Formula (I-I-II-VII-a-1-A-I-a):

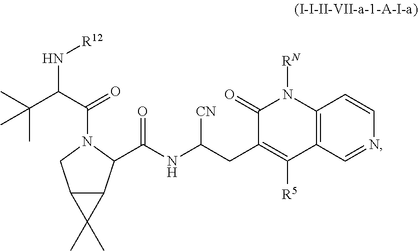

or a pharmaceutically acceptable salt, stereoisomer, solvate, prodrug, isotopic derivative, or tautomer thereof.

In some embodiments, the compound is of Formula (I-I-II-VII-a-1-A-I-a*):

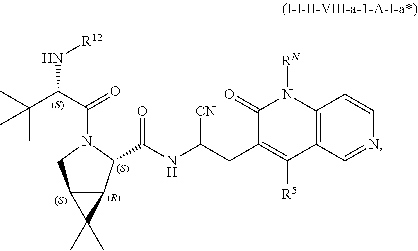

or a pharmaceutically acceptable salt, stereoisomer, solvate, prodrug, isotopic derivative, or tautomer thereof.

In some embodiments, the compound is of Formula (I-I-II-VIII):

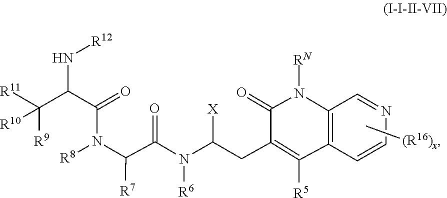

or a pharmaceutically acceptable salt, stereoisomer, solvate, prodrug, isotopic derivative, or tautomer thereof wherein x is an integer selected from 0, 1, 2, 3 and all other variables are as defined herein.

In some embodiments, the compound is of Formula (I-I-II-VIII-a):

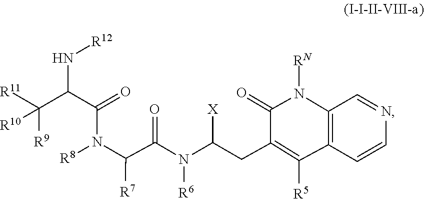

or a pharmaceutically acceptable salt, stereoisomer, solvate, prodrug, isotopic derivative, or tautomer thereof.

In some embodiments, the compound is of Formula (I-I-II-VIII-a-1):

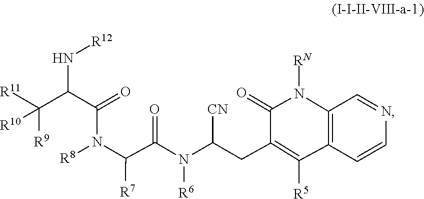

or a pharmaceutically acceptable salt, stereoisomer, solvate, prodrug, isotopic derivative, or tautomer thereof.

In some embodiments, the compound is of Formula (I-I-II-VIII-a-1-A):

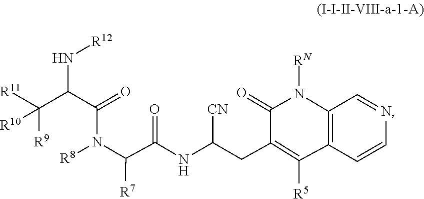

or a pharmaceutically acceptable salt, stereoisomer, solvate, prodrug, isotopic derivative, or tautomer thereof.

In some embodiments, the compound is of Formula (I-I-II-VIII-a-1-A-I):

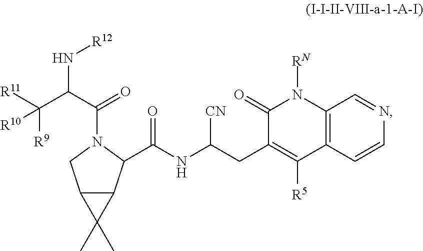

or a pharmaceutically acceptable salt, stereoisomer, solvate, prodrug, isotopic derivative, or tautomer thereof.

In some embodiments, the compound is of Formula (I-I-II-VIII-a-1-A-I-a):

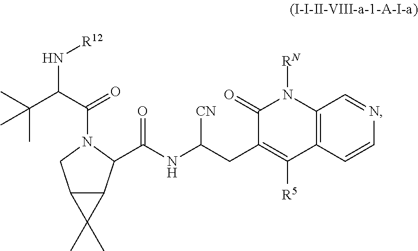

or a pharmaceutically acceptable salt, stereoisomer, solvate, prodrug, isotopic derivative, or tautomer thereof.

In some embodiments, the compound is of Formula (I-I-II-VIII-a-1-A-I-a*):

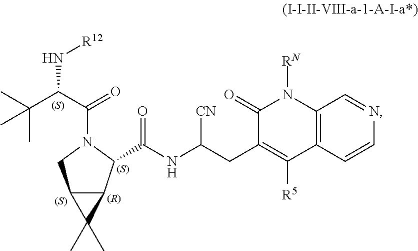

or a pharmaceutically acceptable salt, stereoisomer, solvate, prodrug, isotopic derivative, or tautomer thereof.

In some embodiments, the compound is of Formula (I-A-A):

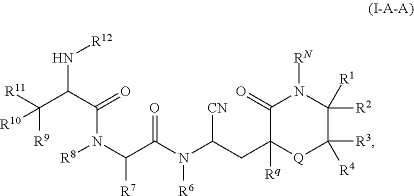

or a pharmaceutically acceptable salt, stereoisomer, solvate, prodrug, isotopic derivative, or tautomer thereof wherein all variables are as defined herein.

In some embodiments, the compound is of Formula (I-A-A-1):

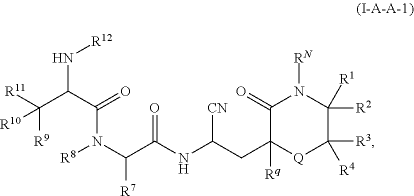

or a pharmaceutically acceptable salt, stereoisomer, solvate, prodrug, isotopic derivative, or tautomer thereof wherein all variables are as defined herein.

In some embodiments, the compound is of Formula (I-A-A-2):

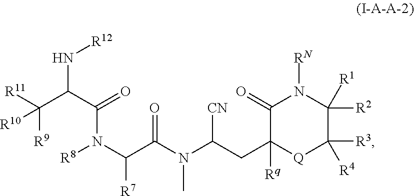

or a pharmaceutically acceptable salt, stereoisomer, solvate, prodrug, isotopic derivative, or tautomer thereof wherein all variables are as defined herein.

In some embodiments, the compound is of Formula (I-A-A-3):

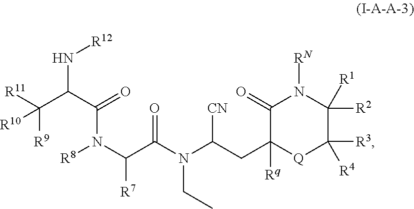

or a pharmaceutically acceptable salt, stereoisomer, solvate, prodrug, isotopic derivative, or tautomer thereof wherein all variables are as defined herein.

In some embodiments, the compound is of Formula (I-A-A-1-1):

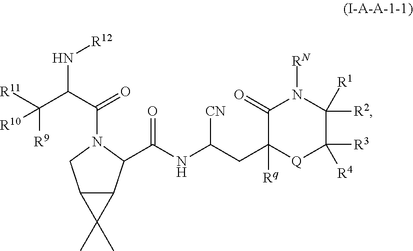

or a pharmaceutically acceptable salt, stereoisomer, solvate, prodrug, isotopic derivative, or tautomer thereof wherein all variables are as defined herein.

In some embodiments, the compound is of Formula (I-A-A-2-1):

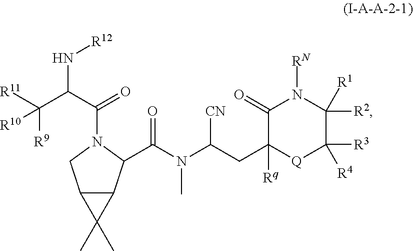

or a pharmaceutically acceptable salt, stereoisomer, solvate, prodrug, isotopic derivative, or tautomer thereof wherein all variables are as defined herein.

In some embodiments, the compound is of Formula (I-A-A-3-1):

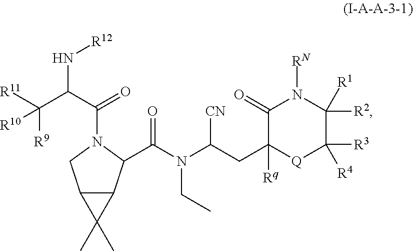

or a pharmaceutically acceptable salt, stereoisomer, solvate, prodrug, isotopic derivative, or tautomer thereof wherein all variables are as defined herein.

In some embodiments, the compound is of Formula (I-A-A-1-1-a):

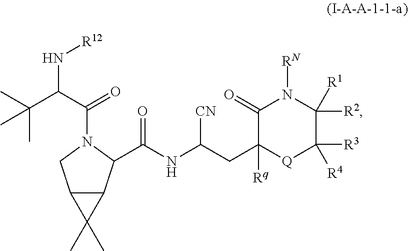

or a pharmaceutically acceptable salt, stereoisomer, solvate, prodrug, isotopic derivative, or tautomer thereof wherein all variables are as defined herein.

In some embodiments, the compound is of Formula (I-A-A-2-1-a):

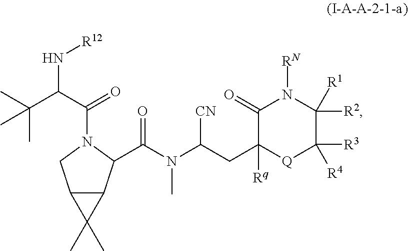

or a pharmaceutically acceptable salt, stereoisomer, solvate, prodrug, isotopic derivative, or tautomer thereof wherein all variables are as defined herein.

In some embodiments, the compound is of Formula (I-A-A-3-1-a):

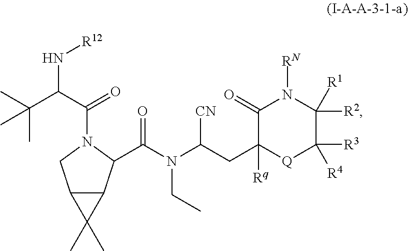

or a pharmaceutically acceptable salt, stereoisomer, solvate, prodrug, isotopic derivative, or tautomer thereof wherein all variables are as defined herein.

In some embodiments, the compound is of Formula (I-A-A-1-1-a*):

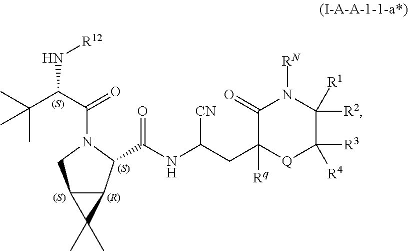

or a pharmaceutically acceptable salt, stereoisomer, solvate, prodrug, isotopic derivative, or tautomer thereof wherein all variables are as defined herein.

In some embodiments, the compound is of Formula (I-A-A-2-1-a*):

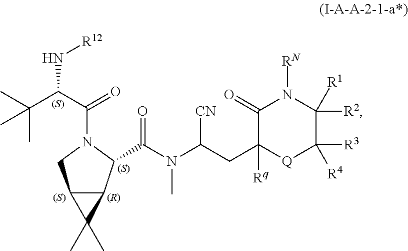

or a pharmaceutically acceptable salt, stereoisomer, solvate, prodrug, isotopic derivative, or tautomer thereof wherein all variables are as defined herein.

In some embodiments, the compound is of Formula (I-A-A-3-1-a*):

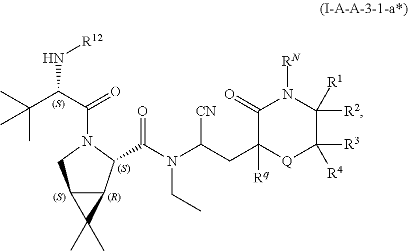

or a pharmaceutically acceptable salt, stereoisomer, solvate, prodrug, isotopic derivative, or tautomer thereof wherein all variables are as defined herein.

In some embodiments, the compound is of Formula (I-A-A-1-2):

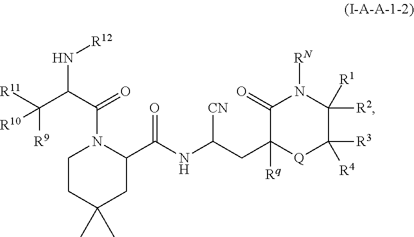

or a pharmaceutically acceptable salt, stereoisomer, solvate, prodrug, isotopic derivative, or tautomer thereof wherein all variables are as defined herein.

In some embodiments, the compound is of Formula (I-A-A-2-2):

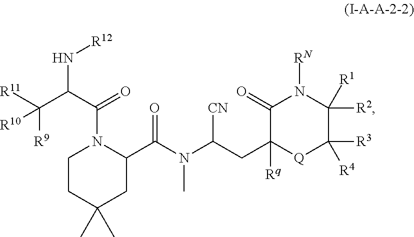

or a pharmaceutically acceptable salt, stereoisomer, solvate, prodrug, isotopic derivative, or tautomer thereof wherein all variables are as defined herein.

In some embodiments, the compound is of Formula (I-A-A-3-2):

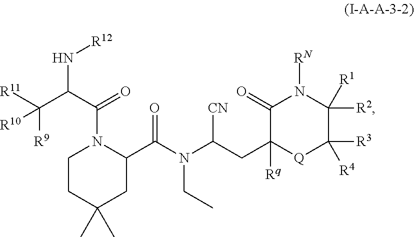

or a pharmaceutically acceptable salt, stereoisomer, solvate, prodrug, isotopic derivative, or tautomer thereof wherein all variables are as defined herein.

In some embodiments, the compound is of Formula (I-A-A-1-2-a):

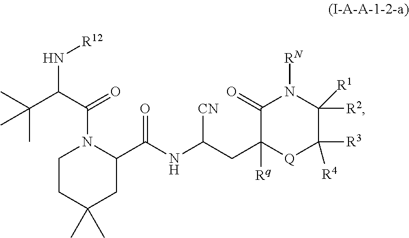

or a pharmaceutically acceptable salt, stereoisomer, solvate, prodrug, isotopic derivative, or tautomer thereof wherein all variables are as defined herein.

In some embodiments, the compound is of Formula (I-A-A-2-2-a):

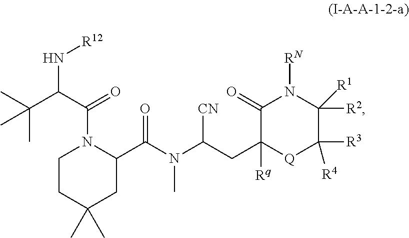

or a pharmaceutically acceptable salt, stereoisomer, solvate, prodrug, isotopic derivative, or tautomer thereof wherein all variables are as defined herein.

In some embodiments, the compound is of Formula (I-A-A-3-2-a):

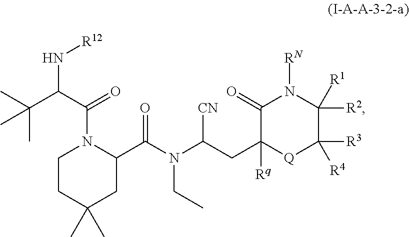

or a pharmaceutically acceptable salt, stereoisomer, solvate, prodrug, isotopic derivative, or tautomer thereof wherein all variables are as defined herein.

In some embodiments, the compound is of Formula (I-A-A-1-2-a*):

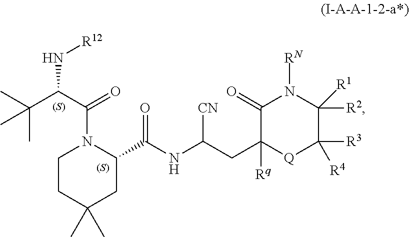

or a pharmaceutically acceptable salt, stereoisomer, solvate, prodrug, isotopic derivative, or tautomer thereof wherein all variables are as defined herein.

In some embodiments, the compound is of Formula (I-A-A-2-2-a*):

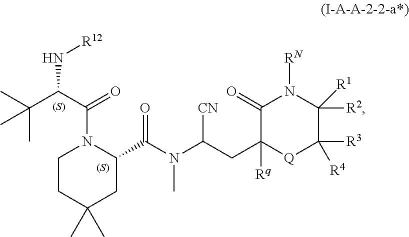

or a pharmaceutically acceptable salt, stereoisomer, solvate, prodrug, isotopic derivative, or tautomer thereof wherein all variables are as defined herein.

In some embodiments, the compound is of Formula (I-A-A-3-2-a*)

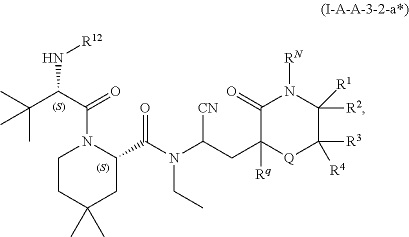

or a pharmaceutically acceptable salt, stereoisomer, solvate, prodrug, isotopic derivative, or tautomer thereof wherein all variables are as defined herein.

In some embodiments, the compound is of Formula (I-A-A-1-3):

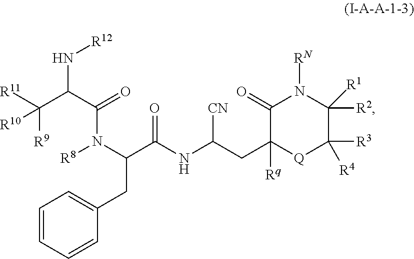

or a pharmaceutically acceptable salt, stereoisomer, solvate, prodrug, isotopic derivative, or tautomer thereof wherein all variables are as defined herein.

In some embodiments, the compound is of Formula (I-A-A-2-3):

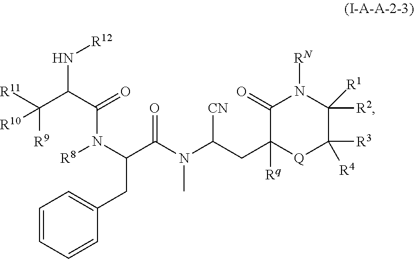

or a pharmaceutically acceptable salt, stereoisomer, solvate, prodrug, isotopic derivative, or tautomer thereof wherein all variables are as defined herein.

In some embodiments, the compound is of Formula (I-A-A-3-3):

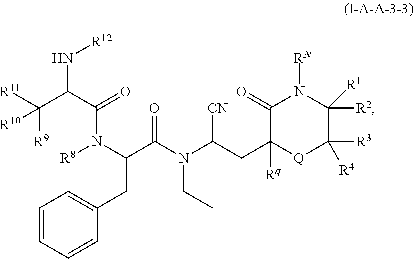

or a pharmaceutically acceptable salt, stereoisomer, solvate, prodrug, isotopic derivative, or tautomer thereof wherein all variables are as defined herein.

In some embodiments, the compound is of Formula (I-A-A-1-3-a):

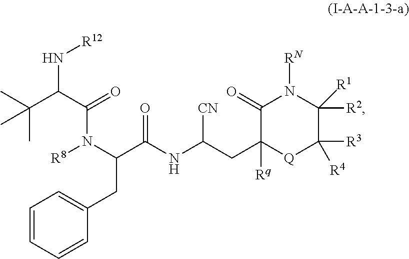

or a pharmaceutically acceptable salt, stereoisomer, solvate, prodrug, isotopic derivative, or tautomer thereof wherein all variables are as defined herein.

In some embodiments, the compound is of Formula (I-A-A-2-3-a):

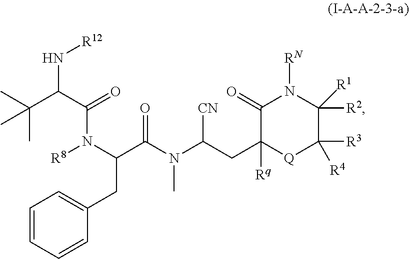

or a pharmaceutically acceptable salt, stereoisomer, solvate, prodrug, isotopic derivative, or tautomer thereof wherein all variables are as defined herein.

In some embodiments, the compound is of Formula (I-A-A-3-3-a):

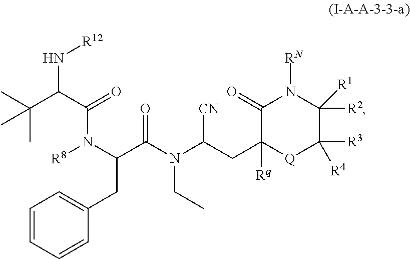

or a pharmaceutically acceptable salt, stereoisomer, solvate, prodrug, isotopic derivative, or tautomer thereof wherein all variables are as defined herein.

In some embodiments, the compound is of Formula (I-A-A-1-3-a*):

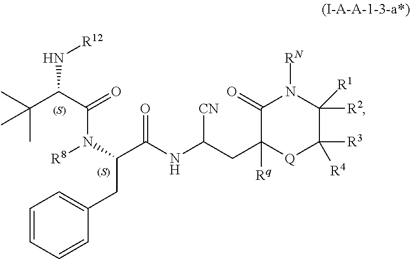

or a pharmaceutically acceptable salt, stereoisomer, solvate, prodrug, isotopic derivative, or tautomer thereof wherein all variables are as defined herein.

In some embodiments, the compound is of Formula (I-A-A-2-3-a*):

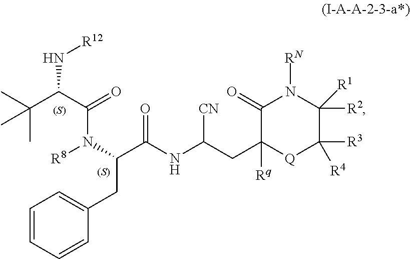

or a pharmaceutically acceptable salt, stereoisomer, solvate, prodrug, isotopic derivative, or tautomer thereof wherein all variables are as defined herein.

In some embodiments, the compound is of Formula (I-A-A-3-3-a*):

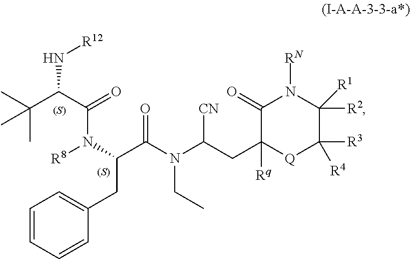

or a pharmaceutically acceptable salt, stereoisomer, solvate, prodrug, isotopic derivative, or tautomer thereof wherein all variables are as defined herein.

In some embodiments, the compound is of Formula (I-A-A-1-4):

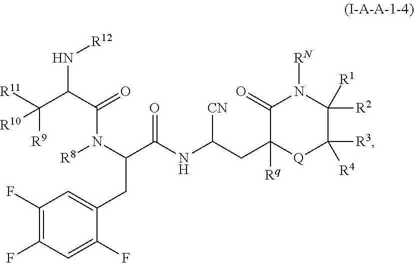

or a pharmaceutically acceptable salt, stereoisomer, solvate, prodrug, isotopic derivative, or tautomer thereof wherein all variables are as defined herein.

In some embodiments, the compound is of Formula (I-A-A-2-4):

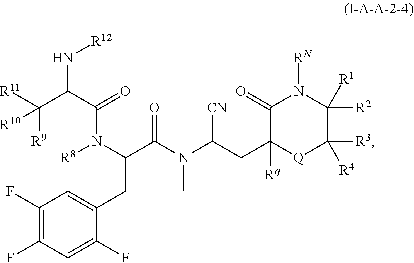

or a pharmaceutically acceptable salt, stereoisomer, solvate, prodrug, isotopic derivative, or tautomer thereof wherein all variables are as defined herein.

In some embodiments, the compound is of Formula (I-A-A-3-4):

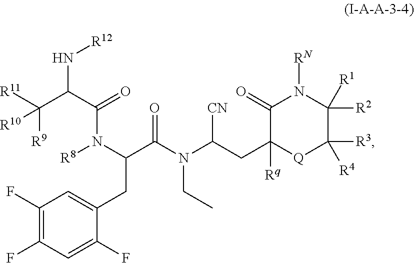

or a pharmaceutically acceptable salt, stereoisomer, solvate, prodrug, isotopic derivative, or tautomer thereof wherein all variables are as defined herein.

In some embodiments, the compound is of Formula (I-A-A-1-4-a):

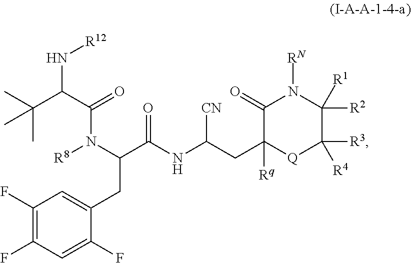

or a pharmaceutically acceptable salt, stereoisomer, solvate, prodrug, isotopic derivative, or tautomer thereof wherein all variables are as defined herein.

In some embodiments, the compound is of Formula (I-A-A-2-4-a):

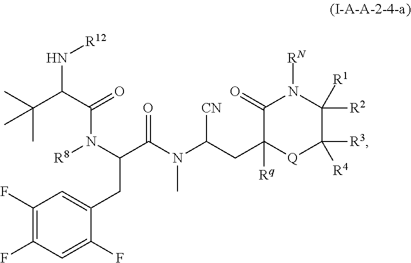

or a pharmaceutically acceptable salt, stereoisomer, solvate, prodrug, isotopic derivative, or tautomer thereof wherein all variables are as defined herein.

In some embodiments, the compound is of Formula (I-A-A-3-4-a):

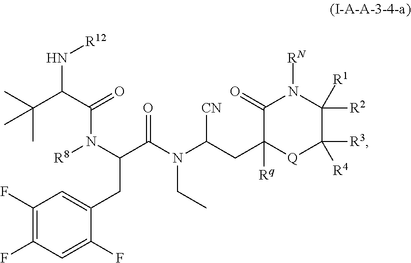

or a pharmaceutically acceptable salt, stereoisomer, solvate, prodrug, isotopic derivative, or tautomer thereof wherein all variables are as defined herein.

In some embodiments, the compound is of Formula (I-A-A-1-4-a*):

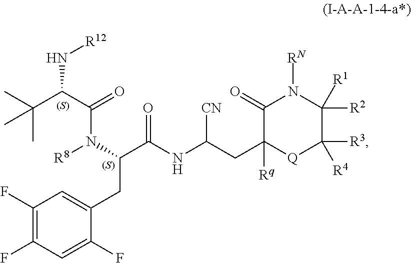

or a pharmaceutically acceptable salt, stereoisomer, solvate, prodrug, isotopic derivative, or tautomer thereof wherein all variables are as defined herein.

In some embodiments, the compound is of Formula (I-A-A-2-4-a*):

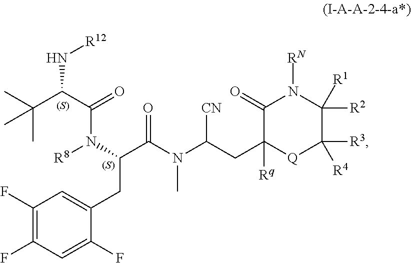

or a pharmaceutically acceptable salt, stereoisomer, solvate, prodrug, isotopic derivative, or tautomer thereof wherein all variables are as defined herein.

In some embodiments, the compound is of Formula (I-A-A-3-4-a*):

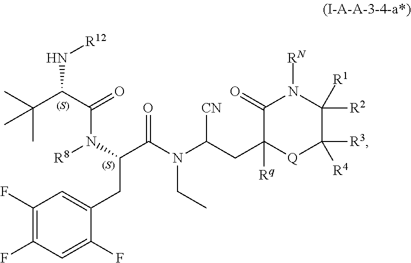

or a pharmaceutically acceptable salt, stereoisomer, solvate, prodrug, isotopic derivative, or tautomer thereof wherein all variables are as defined herein.

In some embodiments, the compound is of Formula (I-A-B):

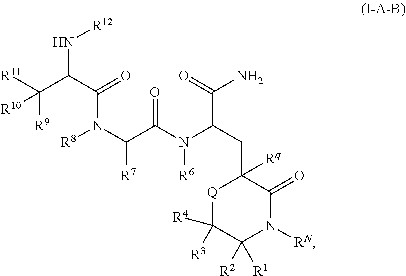

or a pharmaceutically acceptable salt, stereoisomer, solvate, prodrug, isotopic derivative, or tautomer thereof wherein all variables are as defined herein.

In some embodiments, the compound is of Formula (I-A-B-1):

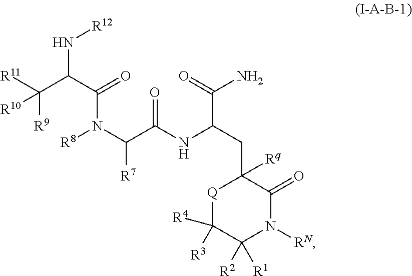

or a pharmaceutically acceptable salt, stereoisomer, solvate, prodrug, isotopic derivative, or tautomer thereof wherein all variables are as defined herein.

In some embodiments, the compound is of Formula (I-A-B-2):

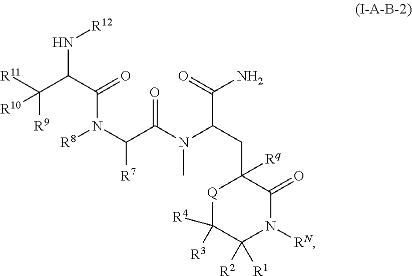

or a pharmaceutically acceptable salt, stereoisomer, solvate, prodrug, isotopic derivative, or tautomer thereof wherein all variables are as defined herein.

In some embodiments, the compound is of Formula (I-A-B-3):

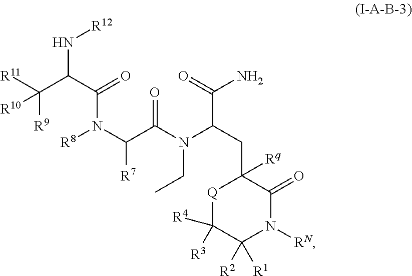

or a pharmaceutically acceptable salt, stereoisomer, solvate, prodrug, isotopic derivative, or tautomer thereof wherein all variables are as defined herein.

In some embodiments, the compound is of Formula (I-A-B-1-1):

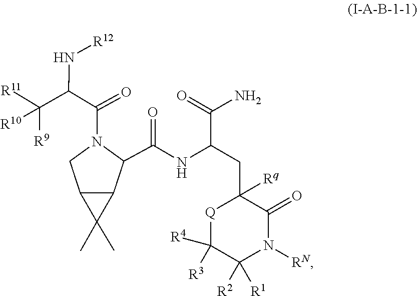

or a pharmaceutically acceptable salt, stereoisomer, solvate, prodrug, isotopic derivative, or tautomer thereof wherein all variables are as defined herein.

In some embodiments, the compound is of Formula (I-A-B-2-1):

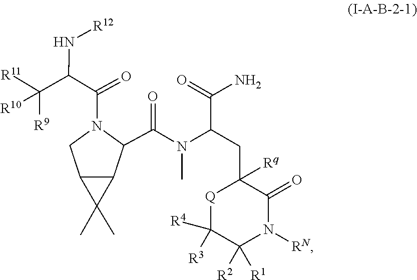

or a pharmaceutically acceptable salt, stereoisomer, solvate, prodrug, isotopic derivative, or tautomer thereof wherein all variables are as defined herein.

In some embodiments, the compound is of Formula (I-A-B-3-1):

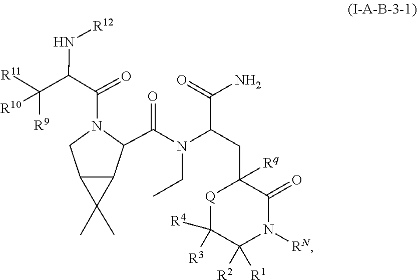

or a pharmaceutically acceptable salt, stereoisomer, solvate, prodrug, isotopic derivative, or tautomer thereof wherein all variables are as defined herein.

In some embodiments, the compound is of Formula (I-A-B-1-1-a):

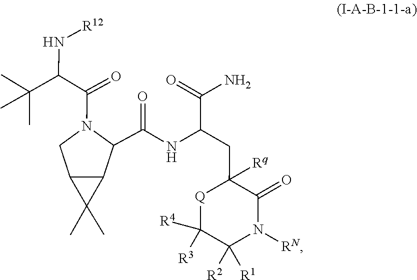

or a pharmaceutically acceptable salt, stereoisomer, solvate, prodrug, isotopic derivative, or tautomer thereof wherein all variables are as defined herein.

In some embodiments, the compound is of Formula (I-A-B-2-1-a):

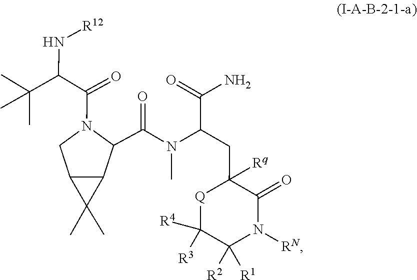

or a pharmaceutically acceptable salt, stereoisomer, solvate, prodrug, isotopic derivative, or tautomer thereof wherein all variables are as defined herein.

In some embodiments, the compound is of Formula (I-A-B-3-1-a):

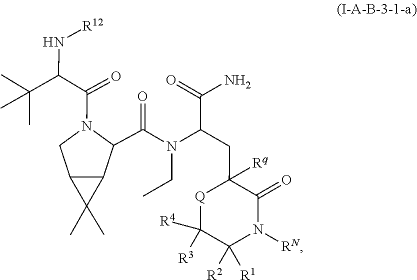

or a pharmaceutically acceptable salt, stereoisomer, solvate, prodrug, isotopic derivative, or tautomer thereof wherein all variables are as defined herein.

In some embodiments, the compound is of Formula (I-A-B-1-1-a*):

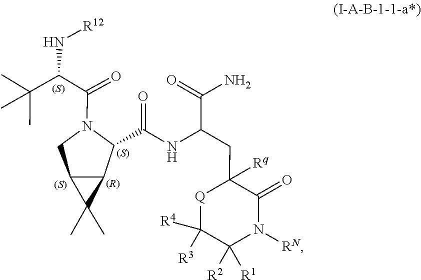

or a pharmaceutically acceptable salt, stereoisomer, solvate, prodrug, isotopic derivative, or tautomer thereof wherein all variables are as defined herein.

In some embodiments, the compound is of Formula (I-A-B-2-1-a*):

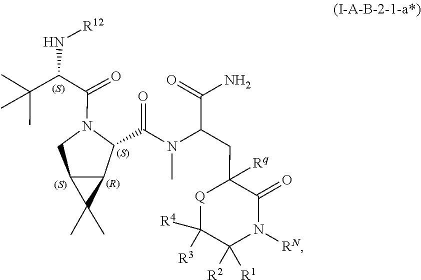

or a pharmaceutically acceptable salt, stereoisomer, solvate, prodrug, isotopic derivative, or tautomer thereof wherein all variables are as defined herein.

In some embodiments, the compound is of Formula (I-A-B-3-1-a*):

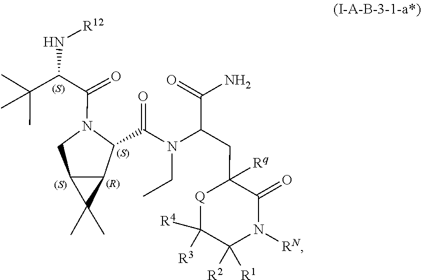

or a pharmaceutically acceptable salt, stereoisomer, solvate, prodrug, isotopic derivative, or tautomer thereof wherein all variables are as defined herein.

In some embodiments, the compound is of Formula (I-A-B-1-2):

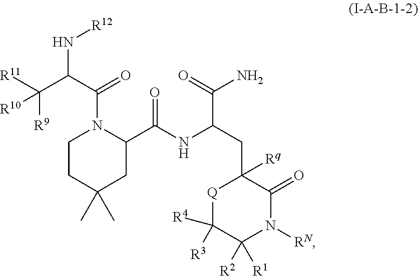

or a pharmaceutically acceptable salt, stereoisomer, solvate, prodrug, isotopic derivative, or tautomer thereof wherein all variables are as defined herein.

In some embodiments, the compound is of Formula (I-A-B-2-2):

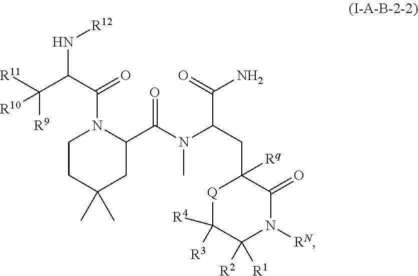

or a pharmaceutically acceptable salt, stereoisomer, solvate, prodrug, isotopic derivative, or tautomer thereof wherein all variables are as defined herein.

In some embodiments, the compound is of Formula (I-A-B-3-2):

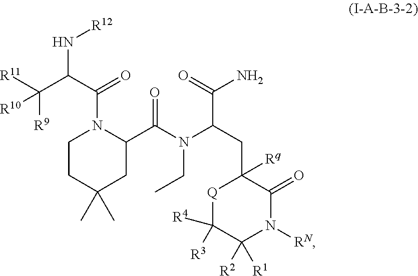

or a pharmaceutically acceptable salt, stereoisomer, solvate, prodrug, isotopic derivative, or tautomer thereof wherein all variables are as defined herein.

In some embodiments, the compound is of Formula (I-A-B-1-2-a):

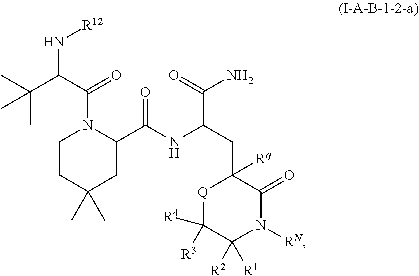

or a pharmaceutically acceptable salt, stereoisomer, solvate, prodrug, isotopic derivative, or tautomer thereof wherein all variables are as defined herein.

In some embodiments, the compound is of Formula (I-A-B-2-2-a):

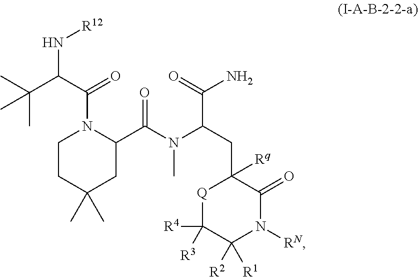

or a pharmaceutically acceptable salt, stereoisomer, solvate, prodrug, isotopic derivative, or tautomer thereof wherein all variables are as defined herein.

In some embodiments, the compound is of Formula (I-A-B-3-2-a):

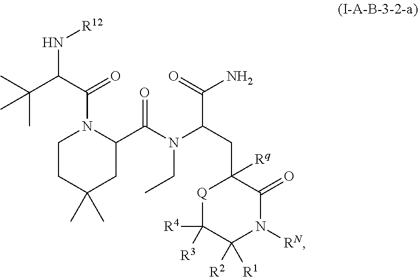

or a pharmaceutically acceptable salt, stereoisomer, solvate, prodrug, isotopic derivative, or tautomer thereof wherein all variables are as defined herein.

In some embodiments, the compound is of Formula (I-A-B-1-2-a*):

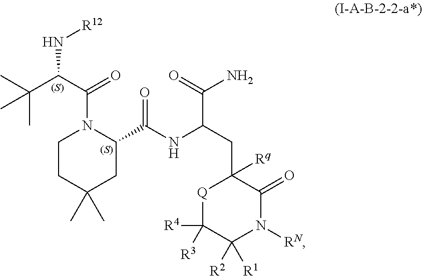

or a pharmaceutically acceptable salt, stereoisomer, solvate, prodrug, isotopic derivative, or tautomer thereof wherein all variables are as defined herein.

In some embodiments, the compound is of Formula (I-A-B-2-2-a*):

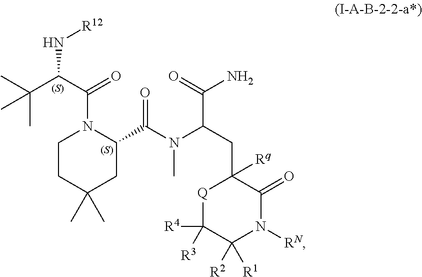

or a pharmaceutically acceptable salt, stereoisomer, solvate, prodrug, isotopic derivative, or tautomer thereof wherein all variables are as defined herein.

In some embodiments, the compound is of Formula (I-A-B-3-2-a*):

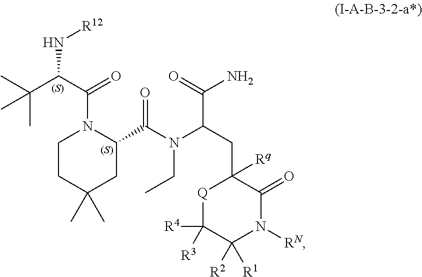

or a pharmaceutically acceptable salt, stereoisomer, solvate, prodrug, isotopic derivative, or tautomer thereof wherein all variables are as defined herein.

In some embodiments, the compound is of Formula (I-A-B-1-3):

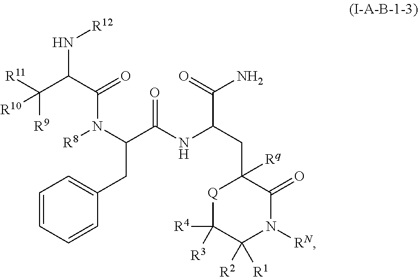

or a pharmaceutically acceptable salt, stereoisomer, solvate, prodrug, isotopic derivative, or tautomer thereof wherein all variables are as defined herein.

In some embodiments, the compound is of Formula (I-A-B-2-3):

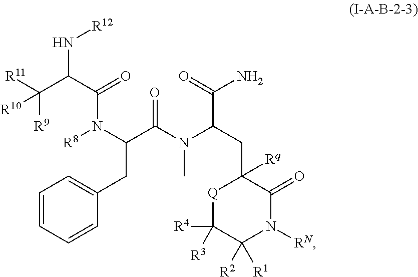

or a pharmaceutically acceptable salt, stereoisomer, solvate, prodrug, isotopic derivative, or tautomer thereof wherein all variables are as defined herein.

In some embodiments, the compound is of Formula (I-A-B-3-3):

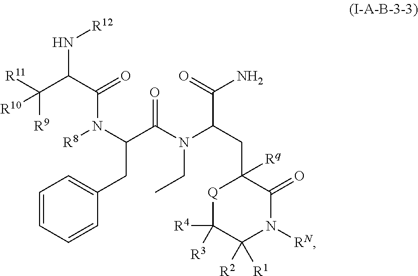

or a pharmaceutically acceptable salt, stereoisomer, solvate, prodrug, isotopic derivative, or tautomer thereof wherein all variables are as defined herein.

In some embodiments, the compound is of Formula (I-A-B-1-3-a):

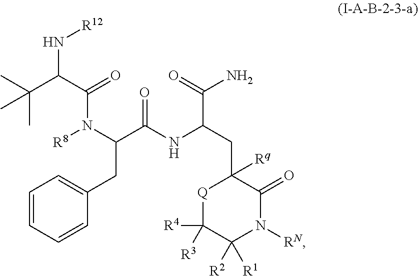

or a pharmaceutically acceptable salt, stereoisomer, solvate, prodrug, isotopic derivative, or tautomer thereof wherein all variables are as defined herein.

In some embodiments, the compound is of Formula (I-A-B-2-3-a):

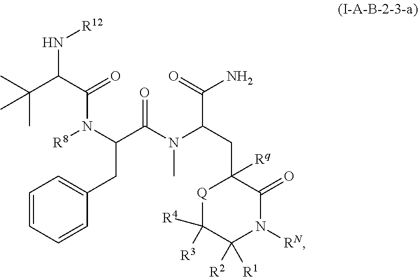

or a pharmaceutically acceptable salt, stereoisomer, solvate, prodrug, isotopic derivative, or tautomer thereof wherein all variables are as defined herein.

In some embodiments, the compound is of Formula (I-A-B-3-3-a):

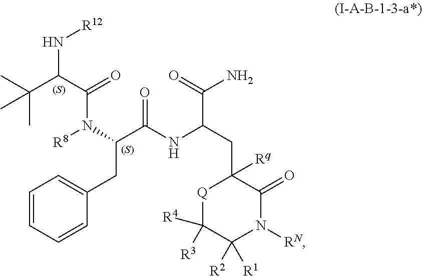

or a pharmaceutically acceptable salt, stereoisomer, solvate, prodrug, isotopic derivative, or tautomer thereof wherein all variables are as defined herein.

In some embodiments, the compound is of Formula (I-A-B-1-3-a*):

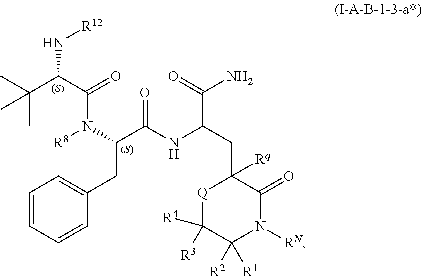

or a pharmaceutically acceptable salt, stereoisomer, solvate, prodrug, isotopic derivative, or tautomer thereof wherein all variables are as defined herein.

In some embodiments, the compound is of Formula (I-A-B-2-3-a*):

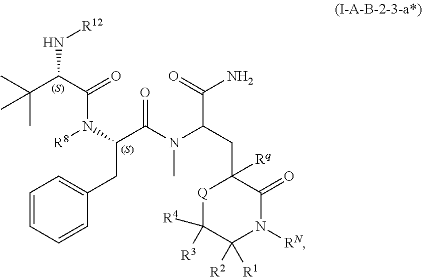

or a pharmaceutically acceptable salt, stereoisomer, solvate, prodrug, isotopic derivative, or tautomer thereof wherein all variables are as defined herein.

In some embodiments, the compound is of Formula (I-A-B-3-3-a*):

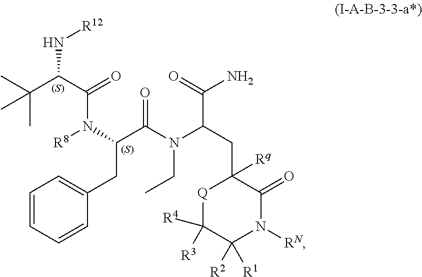

or a pharmaceutically acceptable salt, stereoisomer, solvate, prodrug, isotopic derivative, or tautomer thereof wherein all variables are as defined herein.

In some embodiments, the compound is of Formula (I-A-B-1-4):

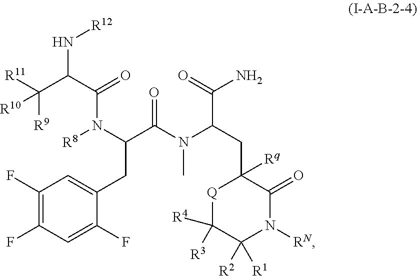

or a pharmaceutically acceptable salt, stereoisomer, solvate, prodrug, isotopic derivative, or tautomer thereof wherein all variables are as defined herein.

In some embodiments, the compound is of Formula (I-A-B-2-4):

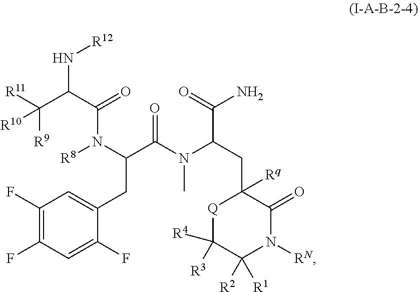

or a pharmaceutically acceptable salt, stereoisomer, solvate, prodrug, isotopic derivative, or tautomer thereof wherein all variables are as defined herein.

In some embodiments, the compound is of Formula (I-A-B-3-4):

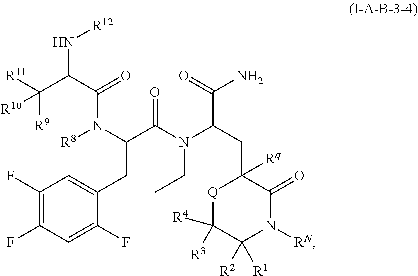

or a pharmaceutically acceptable salt, stereoisomer, solvate, prodrug, isotopic derivative, or tautomer thereof wherein all variables are as defined herein.

In some embodiments, the compound is of Formula (I-A-B-1-4-a):

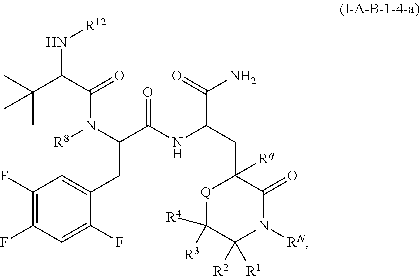

or a pharmaceutically acceptable salt, stereoisomer, solvate, prodrug, isotopic derivative, or tautomer thereof wherein all variables are as defined herein.

In some embodiments, the compound is of Formula (I-A-B-2-4-a):

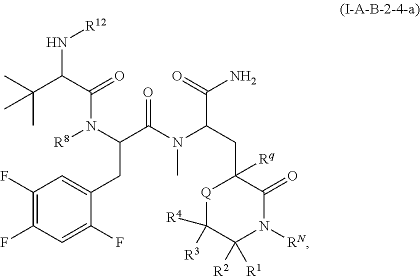

or a pharmaceutically acceptable salt, stereoisomer, solvate, prodrug, isotopic derivative, or tautomer thereof wherein all variables are as defined herein.

In some embodiments, the compound is of Formula (I-A-B-3-4-a):

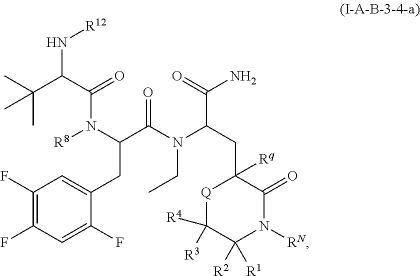

or a pharmaceutically acceptable salt, stereoisomer, solvate, prodrug, isotopic derivative, or tautomer thereof wherein all variables are as defined herein.

In some embodiments, the compound is of Formula (I-A-B-1-4-a*):

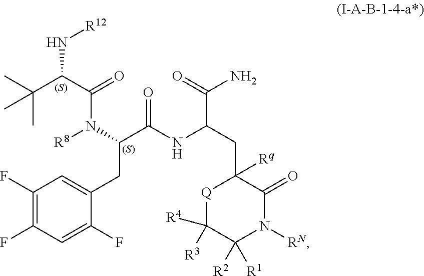

or a pharmaceutically acceptable salt, stereoisomer, solvate, prodrug, isotopic derivative, or tautomer thereof wherein all variables are as defined herein.

In some embodiments, the compound is of Formula (I-A-B-2-4-a*):

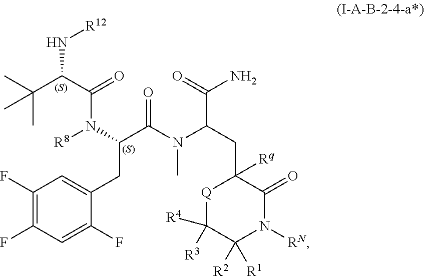

or a pharmaceutically acceptable salt, stereoisomer, solvate, prodrug, isotopic derivative, or tautomer thereof wherein all variables are as defined herein.

In some embodiments, the compound is of Formula (I-A-B-3-4-a*):

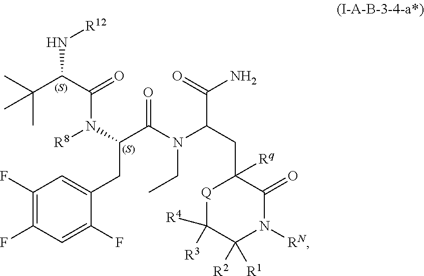

or a pharmaceutically acceptable salt, stereoisomer, solvate, prodrug, isotopic derivative, or tautomer thereof wherein all variables are as defined herein.

In some embodiments, the compound is of Formula (I-I-I-A):

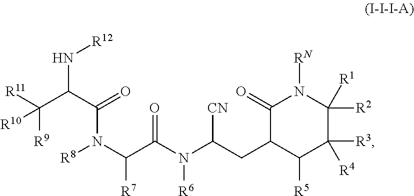

or a pharmaceutically acceptable salt, stereoisomer, solvate, prodrug, isotopic derivative, or tautomer thereof wherein all variables are as defined herein.

In some embodiments, the compound is of Formula (I-I-I-A-1):

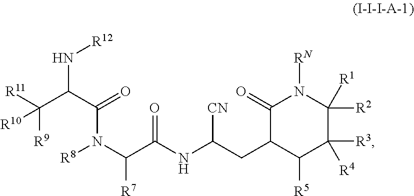

or a pharmaceutically acceptable salt, stereoisomer, solvate, prodrug, isotopic derivative, or tautomer thereof wherein all variables are as defined herein.

In some embodiments, the compound is of Formula (I-I-I-A-2):

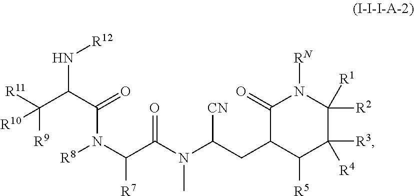

or a pharmaceutically acceptable salt, stereoisomer, solvate, prodrug, isotopic derivative, or tautomer thereof wherein all variables are as defined herein.

In some embodiments, the compound is of Formula (I-I-I-A-1-1):

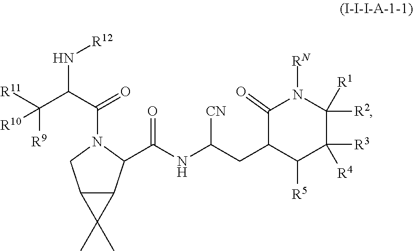

or a pharmaceutically acceptable salt, stereoisomer, solvate, prodrug, isotopic derivative, or tautomer thereof wherein all variables are as defined herein.

In some embodiments, the compound is of Formula (I-I-I-A-2-1):

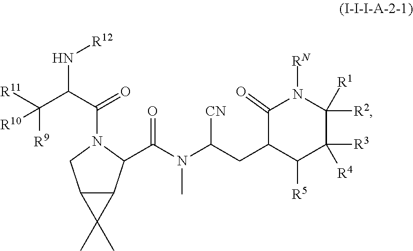

or a pharmaceutically acceptable salt, stereoisomer, solvate, prodrug, isotopic derivative, or tautomer thereof wherein all variables are as defined herein.

In some embodiments, the compound is of Formula (I-I-I-A-1-1-a):

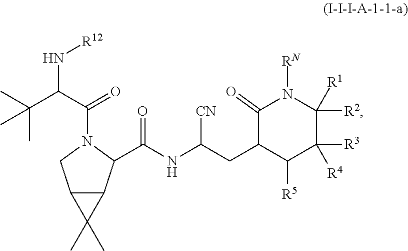

or a pharmaceutically acceptable salt, stereoisomer, solvate, prodrug, isotopic derivative, or tautomer thereof wherein all variables are as defined herein.

In some embodiments, the compound is of Formula (I-I-I-A-2-1-a):

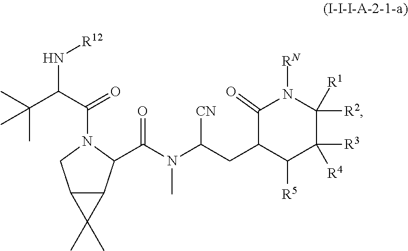

or a pharmaceutically acceptable salt, stereoisomer, solvate, prodrug, isotopic derivative, or tautomer thereof wherein all variables are as defined herein.

In some embodiments, the compound is of Formula (I-I-I-A-1-1-a*):

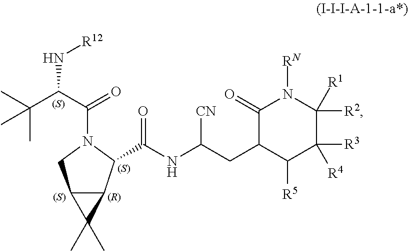

or a pharmaceutically acceptable salt, stereoisomer, solvate, prodrug, isotopic derivative, or tautomer thereof wherein all variables are as defined herein.

In some embodiments, the compound is of Formula (I-I-I-A-2-1-a*):

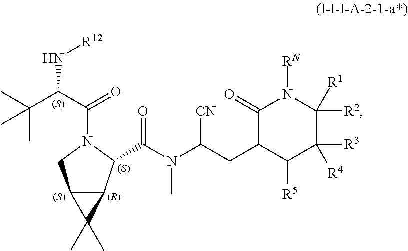

or a pharmaceutically acceptable salt, stereoisomer, solvate, prodrug, isotopic derivative, or tautomer thereof wherein all variables are as defined herein.

In some embodiments, the compound is of Formula (I-I-I-A-1-2):

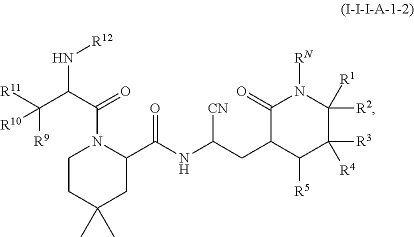

or a pharmaceutically acceptable salt, stereoisomer, solvate, prodrug, isotopic derivative, or tautomer thereof wherein all variables are as defined herein.

In some embodiments, the compound is of Formula (I-I-I-A-2-2):

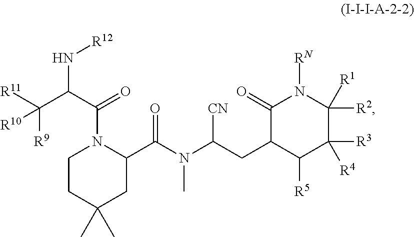

or a pharmaceutically acceptable salt, stereoisomer, solvate, prodrug, isotopic derivative, or tautomer thereof wherein all variables are as defined herein.

In some embodiments, the compound is of Formula (I-I-I-A-1-2-a):

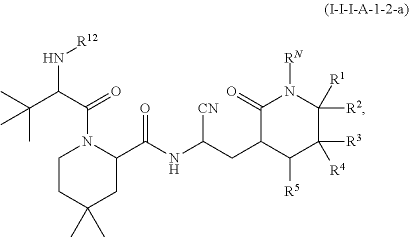

or a pharmaceutically acceptable salt, stereoisomer, solvate, prodrug, isotopic derivative, or tautomer thereof wherein all variables are as defined herein.

In some embodiments, the compound is of Formula (I-I-I-A-2-2-a):

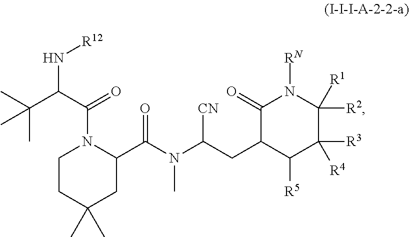

or a pharmaceutically acceptable salt, stereoisomer, solvate, prodrug, isotopic derivative, or tautomer thereof wherein all variables are as defined herein.

In some embodiments, the compound is of Formula (I-I-I-A-1-2-a*):

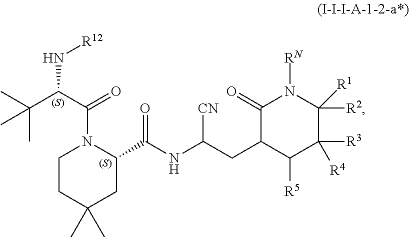

or a pharmaceutically acceptable salt, stereoisomer, solvate, prodrug, isotopic derivative, or tautomer thereof wherein all variables are as defined herein.

In some embodiments, the compound is of Formula (I-I-I-A-2-2-a*):

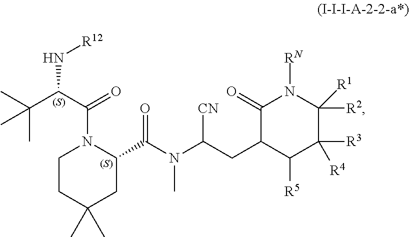

or a pharmaceutically acceptable salt, stereoisomer, solvate, prodrug, isotopic derivative, or tautomer thereof wherein all variables are as defined herein.

In some embodiments, the compound is of Formula (I-I-I-A-1-3):

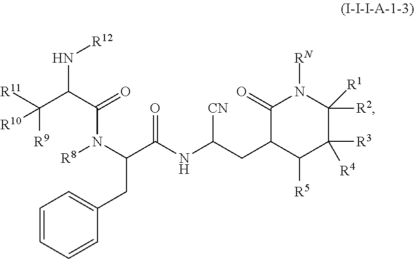

or a pharmaceutically acceptable salt, stereoisomer, solvate, prodrug, isotopic derivative, or tautomer thereof wherein all variables are as defined herein.

In some embodiments, the compound is of Formula (I-I-I-A-2-3):

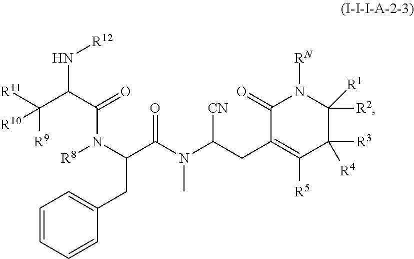

or a pharmaceutically acceptable salt, stereoisomer, solvate, prodrug, isotopic derivative, or tautomer thereof wherein all variables are as defined herein.

In some embodiments, the compound is of Formula (I-I-I-A-1-3-a):

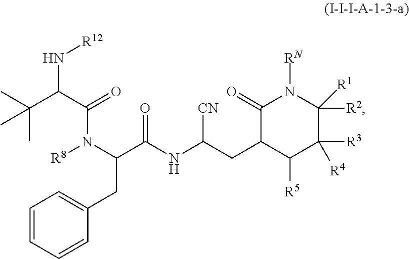

or a pharmaceutically acceptable salt, stereoisomer, solvate, prodrug, isotopic derivative, or tautomer thereof wherein all variables are as defined herein.

In some embodiments, the compound is of Formula (I-I-I-A-2-3-a):

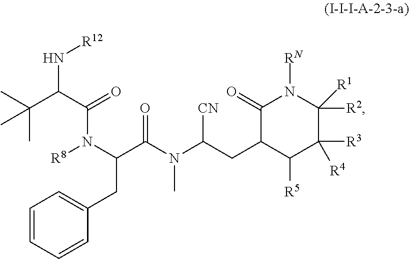

or a pharmaceutically acceptable salt, stereoisomer, solvate, prodrug, isotopic derivative, or tautomer thereof wherein all variables are as defined herein.

In some embodiments, the compound is of Formula (I-I-I-A-1-3-a*):

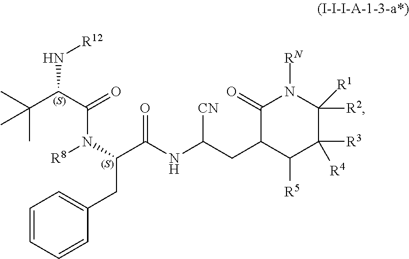

or a pharmaceutically acceptable salt, stereoisomer, solvate, prodrug, isotopic derivative, or tautomer thereof wherein all variables are as defined herein.

In some embodiments, the compound is of Formula (I-I-I-A-2-3-a*):

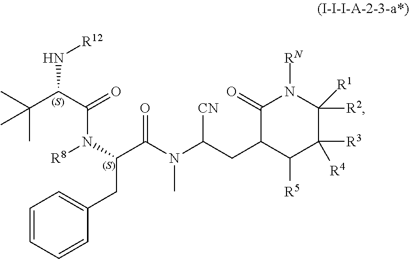

or a pharmaceutically acceptable salt, stereoisomer, solvate, prodrug, isotopic derivative, or tautomer thereof wherein all variables are as defined herein.

In some embodiments, the compound is of Formula (I-I-I-A-1-4):

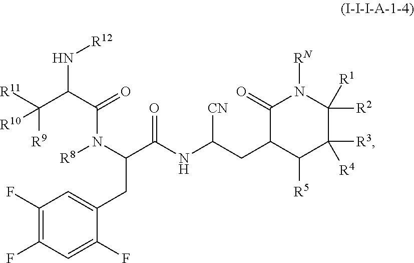

or a pharmaceutically acceptable salt, stereoisomer, solvate, prodrug, isotopic derivative, or tautomer thereof wherein all variables are as defined herein.

In some embodiments, the compound is of Formula (I-I-I-A-2-4):

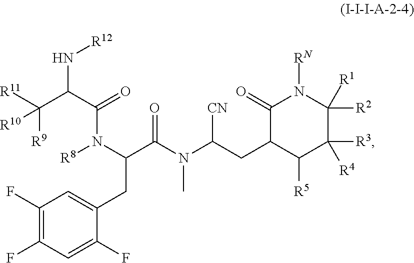

or a pharmaceutically acceptable salt, stereoisomer, solvate, prodrug, isotopic derivative, or tautomer thereof wherein all variables are as defined herein.

In some embodiments, the compound is of Formula (I-I-I-A-1-4-a):

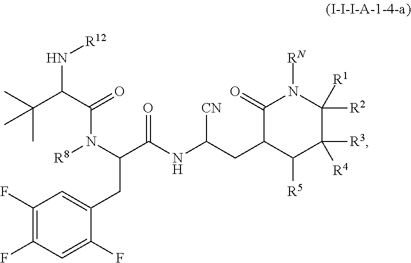

or a pharmaceutically acceptable salt, stereoisomer, solvate, prodrug, isotopic derivative, or tautomer thereof wherein all variables are as defined herein.

In some embodiments, the compound is of Formula (I-I-I-A-2-4-a):

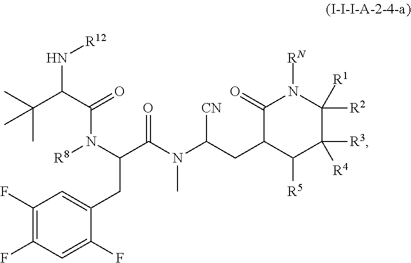

or a pharmaceutically acceptable salt, stereoisomer, solvate, prodrug, isotopic derivative, or tautomer thereof wherein all variables are as defined herein.

In some embodiments, the compound is of Formula (I-I-I-A-1-4-a*):

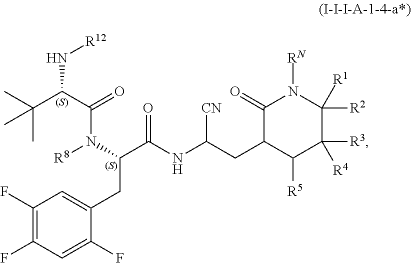

or a pharmaceutically acceptable salt, stereoisomer, solvate, prodrug, isotopic derivative, or tautomer thereof wherein all variables are as defined herein.

In some embodiments, the compound is of Formula (I-I-I-A-2-4-a*):

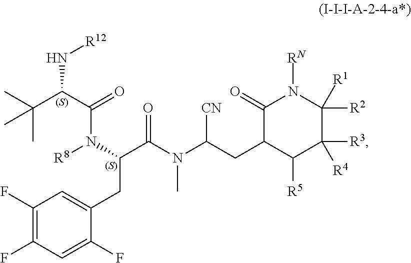

or a pharmaceutically acceptable salt, stereoisomer, solvate, prodrug, isotopic derivative, or tautomer thereof wherein all variables are as defined herein.

In some embodiments, the compound is of Formula (I-I-II-A):

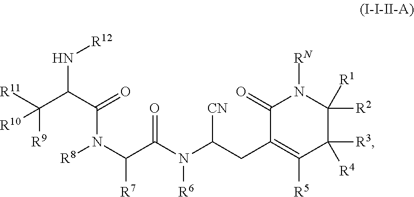

or a pharmaceutically acceptable salt, stereoisomer, solvate, prodrug, isotopic derivative, or tautomer thereof wherein all variables are as defined herein.

In some embodiments, the compound is of Formula (I-I-II-A-1):

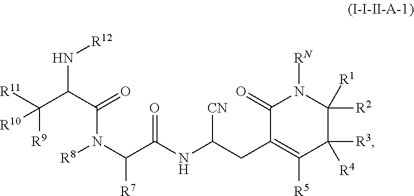

or a pharmaceutically acceptable salt, stereoisomer, solvate, prodrug, isotopic derivative, or tautomer thereof wherein all variables are as defined herein.

In some embodiments, the compound is of Formula (I-I-II-A-2):

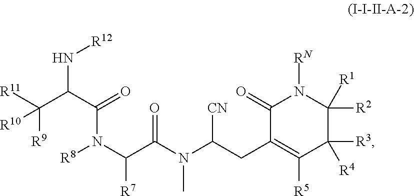

or a pharmaceutically acceptable salt, stereoisomer, solvate, prodrug, isotopic derivative, or tautomer thereof wherein all variables are as defined herein.

In some embodiments, the compound is of Formula (I-A-A-1-1):

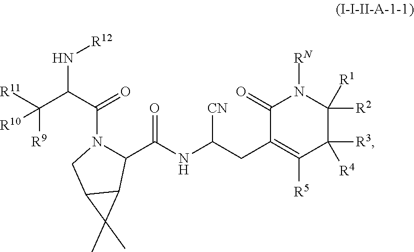

or a pharmaceutically acceptable salt, stereoisomer, solvate, prodrug, isotopic derivative, or tautomer thereof wherein all variables are as defined herein.

In some embodiments, the compound is of Formula (I-A-A-2-1):

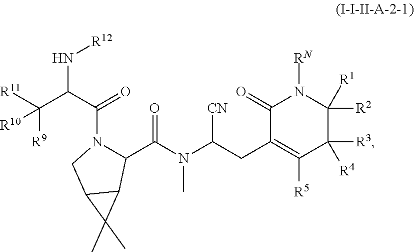

or a pharmaceutically acceptable salt, stereoisomer, solvate, prodrug, isotopic derivative, or tautomer thereof wherein all variables are as defined herein.

In some embodiments, the compound is of Formula (I-I-II-A-1-1-a):

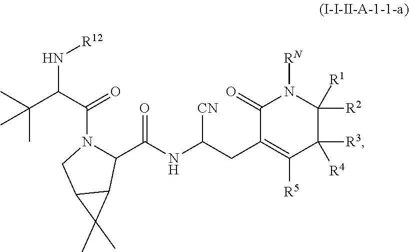

or a pharmaceutically acceptable salt, stereoisomer, solvate, prodrug, isotopic derivative, or tautomer thereof wherein all variables are as defined herein.

In some embodiments, the compound is of Formula (I-I-II-A-2-1-a):

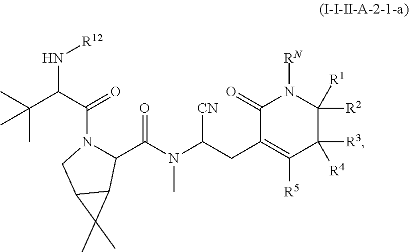

or a pharmaceutically acceptable salt, stereoisomer, solvate, prodrug, isotopic derivative, or tautomer thereof wherein all variables are as defined herein.

In some embodiments, the compound is of Formula (I-I-II-A-1-1-a*):

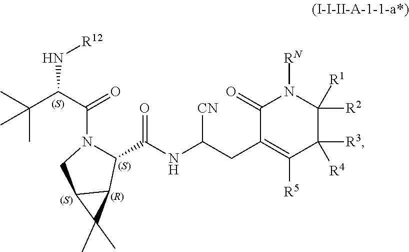

or a pharmaceutically acceptable salt, stereoisomer, solvate, prodrug, isotopic derivative, or tautomer thereof wherein all variables are as defined herein.

In some embodiments, the compound is of Formula (I-I-II-A-2-1-a*):

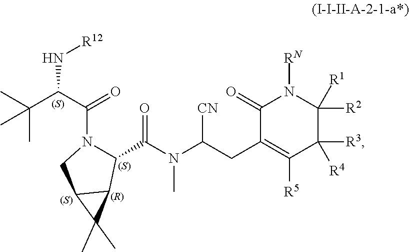

or a pharmaceutically acceptable salt, stereoisomer, solvate, prodrug, isotopic derivative, or tautomer thereof wherein all variables are as defined herein.

In some embodiments, the compound is of Formula (I-I-II-A-1-2):

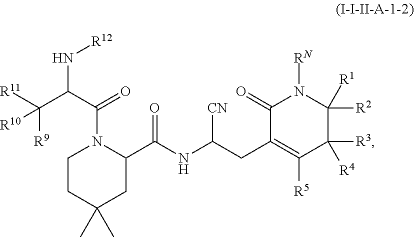

or a pharmaceutically acceptable salt, stereoisomer, solvate, prodrug, isotopic derivative, or tautomer thereof wherein all variables are as defined herein.

In some embodiments, the compound is of Formula (I-I-II-A-2-2):

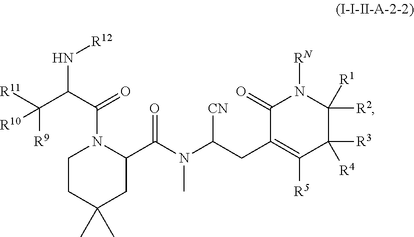

or a pharmaceutically acceptable salt, stereoisomer, solvate, prodrug, isotopic derivative, or tautomer thereof wherein all variables are as defined herein.

In some embodiments, the compound is of Formula (I-I-II-A-1-2-a):

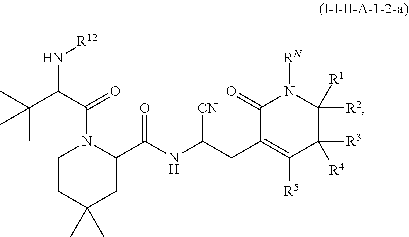

or a pharmaceutically acceptable salt, stereoisomer, solvate, prodrug, isotopic derivative, or tautomer thereof wherein all variables are as defined herein.

In some embodiments, the compound is of Formula (I-I-II-A-2-2-a):

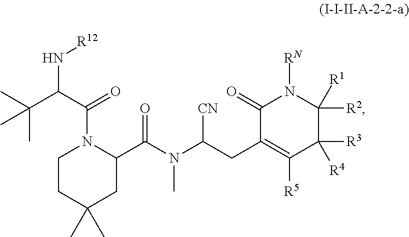

or a pharmaceutically acceptable salt, stereoisomer, solvate, prodrug, isotopic derivative, or tautomer thereof wherein all variables are as defined herein.

In some embodiments, the compound is of Formula I-I-II-A-1-2-a*)

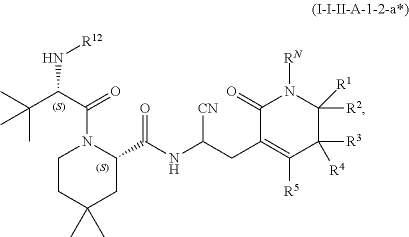

or a pharmaceutically acceptable salt, stereoisomer, solvate, prodrug, isotopic derivative, or tautomer thereof wherein all variables are as defined herein.

In some embodiments, the compound is of Formula (I-I-II-A-2-2-a*):

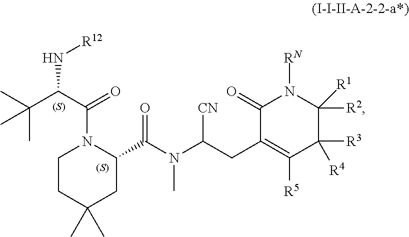

or a pharmaceutically acceptable salt, stereoisomer, solvate, prodrug, isotopic derivative, or tautomer thereof wherein all variables are as defined herein.

In some embodiments, the compound is of Formula (I-I-II-A-1-3):

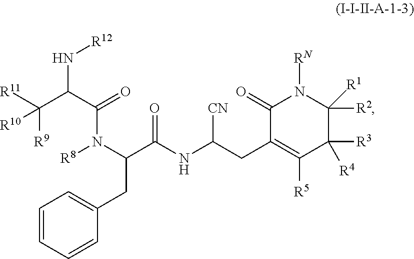

or a pharmaceutically acceptable salt, stereoisomer, solvate, prodrug, isotopic derivative, or tautomer thereof wherein all variables are as defined herein.

In some embodiments, the compound is of Formula (I-I-II-A-2-3):

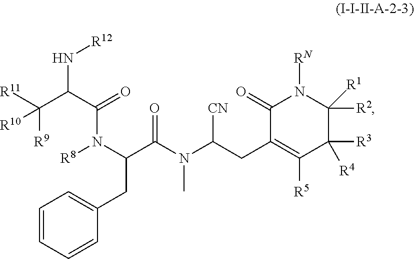

or a pharmaceutically acceptable salt, stereoisomer, solvate, prodrug, isotopic derivative, or tautomer thereof wherein all variables are as defined herein.

In some embodiments, the compound is of Formula (I-I-II-A-1-3-a):

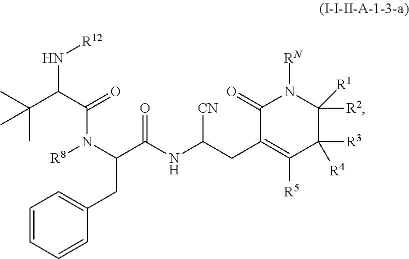

or a pharmaceutically acceptable salt, stereoisomer, solvate, prodrug, isotopic derivative, or tautomer thereof wherein all variables are as defined herein.

In some embodiments, the compound is of Formula (I-I-II-A-2-3-a):

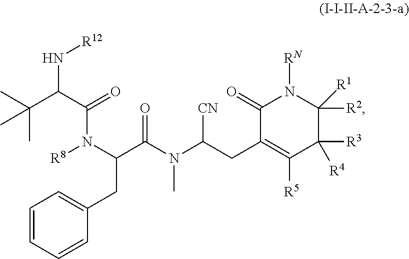

or a pharmaceutically acceptable salt, stereoisomer, solvate, prodrug, isotopic derivative, or tautomer thereof wherein all variables are as defined herein.

In some embodiments, the compound is of Formula (I-I-II-A-1-3-a*):

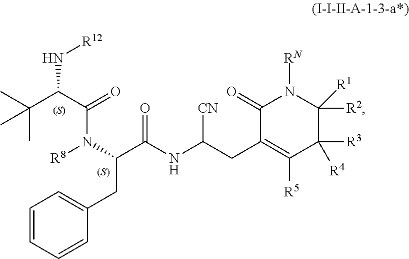

or a pharmaceutically acceptable salt, stereoisomer, solvate, prodrug, isotopic derivative, or tautomer thereof wherein all variables are as defined herein.

In some embodiments, the compound is of Formula (I-I-II-A-2-3-a*):

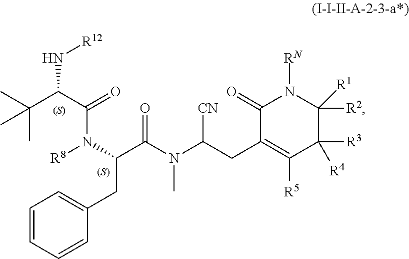

or a pharmaceutically acceptable salt, stereoisomer, solvate, prodrug, isotopic derivative, or tautomer thereof wherein all variables are as defined herein.

In some embodiments, the compound is of Formula (I-I-II-A-1-4):

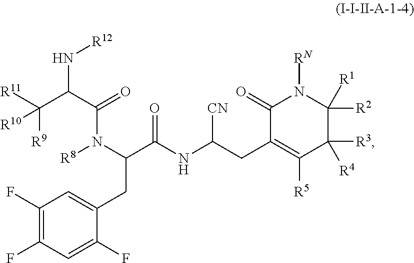

or a pharmaceutically acceptable salt, stereoisomer, solvate, prodrug, isotopic derivative, or tautomer thereof wherein all variables are as defined herein.

In some embodiments, the compound is of Formula (I-I-II-A-2-4):

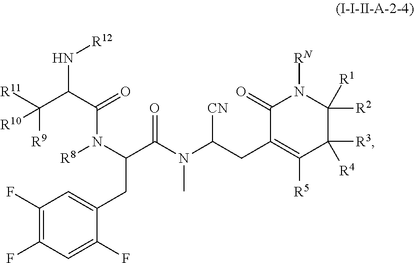

or a pharmaceutically acceptable salt, stereoisomer, solvate, prodrug, isotopic derivative, or tautomer thereof wherein all variables are as defined herein.

In some embodiments, the compound is of Formula (I-I-II-A-1-4-a):

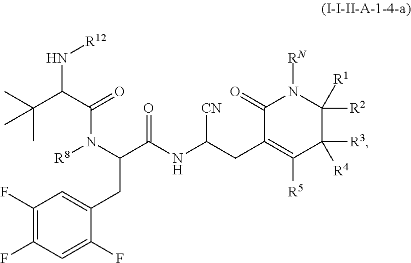

or a pharmaceutically acceptable salt, stereoisomer, solvate, prodrug, isotopic derivative, or tautomer thereof wherein all variables are as defined herein.

In some embodiments, the compound is of Formula (I-I-II-A-2-4-a):

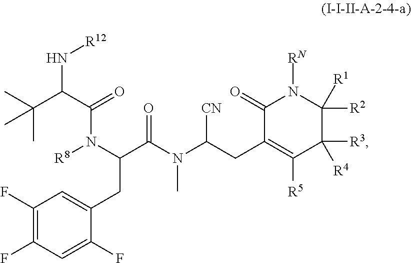

or a pharmaceutically acceptable salt, stereoisomer, solvate, prodrug, isotopic derivative, or tautomer thereof wherein all variables are as defined herein.

In some embodiments, the compound is of Formula (I-I-II-A-1-4-a*):

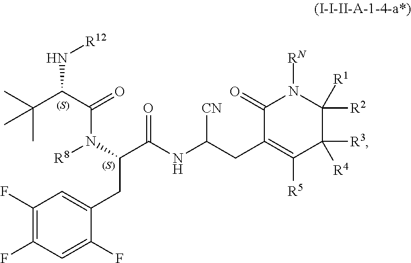

or a pharmaceutically acceptable salt, stereoisomer, solvate, prodrug, isotopic derivative, or tautomer thereof wherein all variables are as defined herein.

In some embodiments, the compound is of Formula (I-I-II-A-2-4-a*):

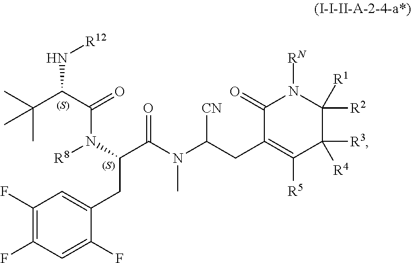

or a pharmaceutically acceptable salt, stereoisomer, solvate, prodrug, isotopic derivative, or tautomer thereof wherein all variables are as defined herein.

In some embodiments, the compound is of Formula (I-II-I-A):

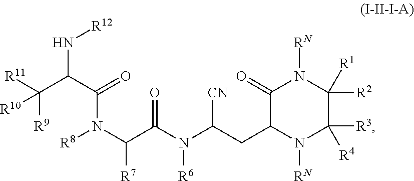

or a pharmaceutically acceptable salt, stereoisomer, solvate, prodrug, isotopic derivative, or tautomer thereof wherein all variables are as defined herein.

In some embodiments, the compound is of Formula (I-II-I-A-1):

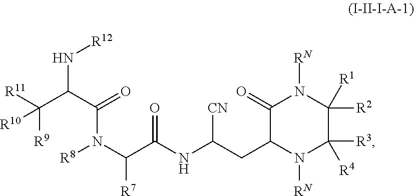

or a pharmaceutically acceptable salt, stereoisomer, solvate, prodrug, isotopic derivative, or tautomer thereof wherein all variables are as defined herein.

In some embodiments, the compound is of Formula (I-II-I-A-2):

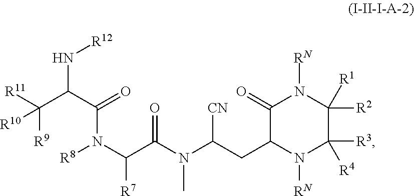

or a pharmaceutically acceptable salt, stereoisomer, solvate, prodrug, isotopic derivative, or tautomer thereof wherein all variables are as defined herein.

In some embodiments, the compound is of Formula (I-II-I-A-1-1):

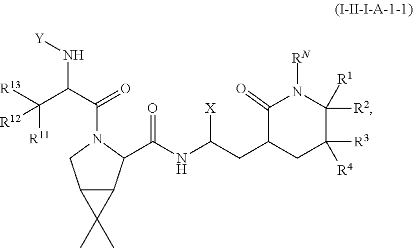

or a pharmaceutically acceptable salt, stereoisomer, solvate, prodrug, isotopic derivative, or tautomer thereof wherein all variables are as defined herein.

In some embodiments, the compound is of Formula (I-II-I-A-2-1):

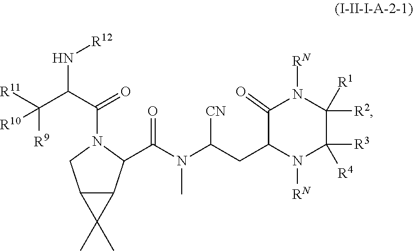

or a pharmaceutically acceptable salt, stereoisomer, solvate, prodrug, isotopic derivative, or tautomer thereof wherein all variables are as defined herein.

In some embodiments, the compound is of Formula (I-II-I-A-1-1-a):

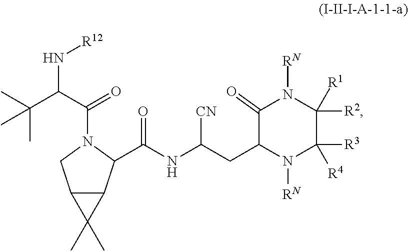

or a pharmaceutically acceptable salt, stereoisomer, solvate, prodrug, isotopic derivative, or tautomer thereof wherein all variables are as defined herein.

In some embodiments, the compound is of Formula (I-II-I-A-2-1-a):

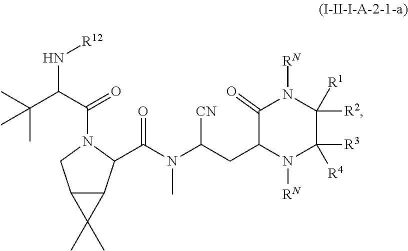

or a pharmaceutically acceptable salt, stereoisomer, solvate, prodrug, isotopic derivative, or tautomer thereof wherein all variables are as defined herein.

In some embodiments, the compound is of Formula (I-II-I-A-1-1-a*):

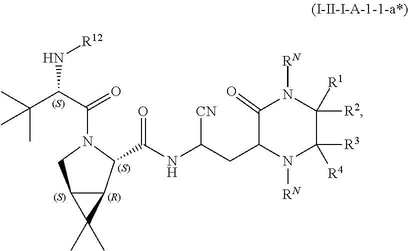

or a pharmaceutically acceptable salt, stereoisomer, solvate, prodrug, isotopic derivative, or tautomer thereof wherein all variables are as defined herein.

In some embodiments, the compound is of Formula (I-II-I-A-2-1-a*):

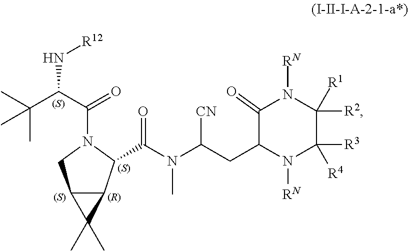

or a pharmaceutically acceptable salt, stereoisomer, solvate, prodrug, isotopic derivative, or tautomer thereof wherein all variables are as defined herein.

In some embodiments, the compound is of Formula (I-II-I-A-1-2):

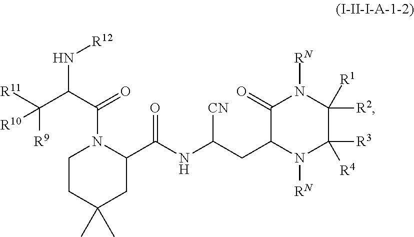

or a pharmaceutically acceptable salt, stereoisomer, solvate, prodrug, isotopic derivative, or tautomer thereof wherein all variables are as defined herein.

In some embodiments, the compound is of Formula (I-II-I-A-2-2):

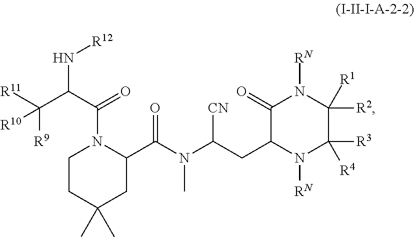

or a pharmaceutically acceptable salt, stereoisomer, solvate, prodrug, isotopic derivative, or tautomer thereof wherein all variables are as defined herein.

In some embodiments, the compound is of Formula (I-II-I-A-1-2-a):

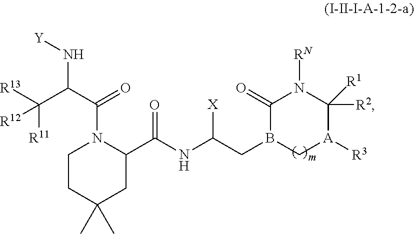

or a pharmaceutically acceptable salt, stereoisomer, solvate, prodrug, isotopic derivative, or tautomer thereof wherein all variables are as defined herein.

In some embodiments, the compound is of Formula (I-II-I-A-2-2-a):

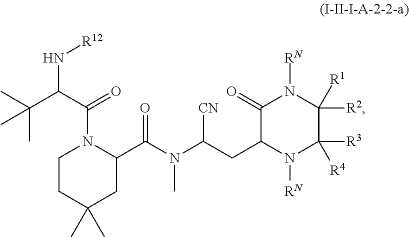

or a pharmaceutically acceptable salt, stereoisomer, solvate, prodrug, isotopic derivative, or tautomer thereof wherein all variables are as defined herein.

In some embodiments, the compound is of Formula (I-II-I-A-1-2-a*):

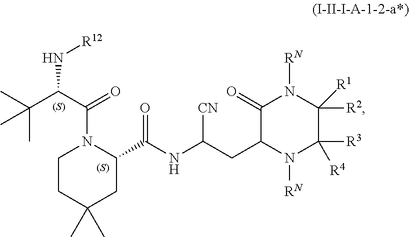

or a pharmaceutically acceptable salt, stereoisomer, solvate, prodrug, isotopic derivative, or tautomer thereof wherein all variables are as defined herein.

In some embodiments, the compound is of Formula (I-II-I-A-2-2-a*):

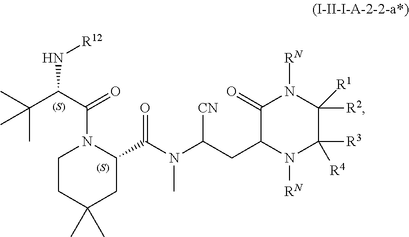

or a pharmaceutically acceptable salt, stereoisomer, solvate, prodrug, isotopic derivative, or tautomer thereof wherein all variables are as defined herein.

In some embodiments, the compound is of Formula (I-II-I-A-1-3):

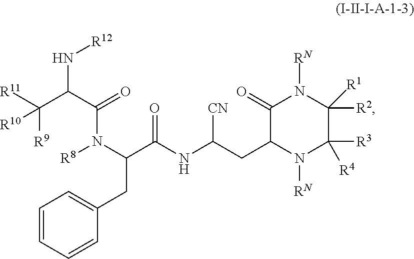

or a pharmaceutically acceptable salt, stereoisomer, solvate, prodrug, isotopic derivative, or tautomer thereof wherein all variables are as defined herein.

In some embodiments, the compound is of Formula (I-II-I-A-2-3):

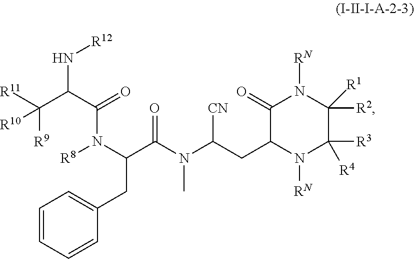

or a pharmaceutically acceptable salt, stereoisomer, solvate, prodrug, isotopic derivative, or tautomer thereof wherein all variables are as defined herein.

In some embodiments, the compound is of Formula (I-II-I-A-1-3-a):

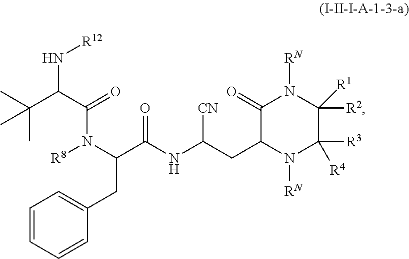

or a pharmaceutically acceptable salt, stereoisomer, solvate, prodrug, isotopic derivative, or tautomer thereof wherein all variables are as defined herein.

In some embodiments, the compound is of Formula (I-II-I-A-2-3-a):

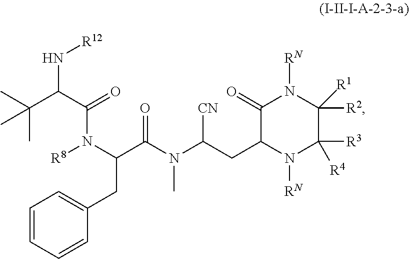

or a pharmaceutically acceptable salt, stereoisomer, solvate, prodrug, isotopic derivative, or tautomer thereof wherein all variables are as defined herein.

In some embodiments, the compound is of Formula (I-II-I-A-1-3-a*):

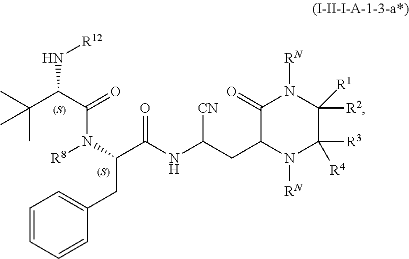

or a pharmaceutically acceptable salt, stereoisomer, solvate, prodrug, isotopic derivative, or tautomer thereof wherein all variables are as defined herein.

In some embodiments, the compound is of Formula (I-II-I-A-2-3-a*):

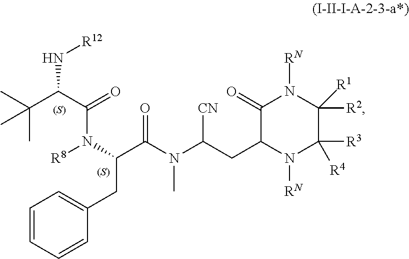

or a pharmaceutically acceptable salt, stereoisomer, solvate, prodrug, isotopic derivative, or tautomer thereof wherein all variables are as defined herein.

In some embodiments, the compound is of Formula (I-II-I-A-1-4):

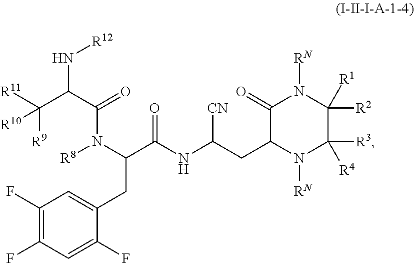

or a pharmaceutically acceptable salt, stereoisomer, solvate, prodrug, isotopic derivative, or tautomer thereof wherein all variables are as defined herein.

In some embodiments, the compound is of Formula (I-II-I-A-2-4):

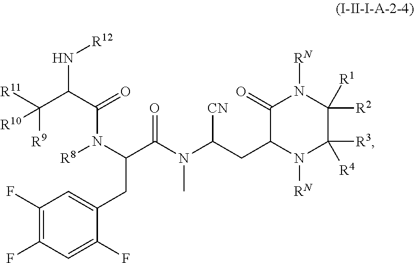

or a pharmaceutically acceptable salt, stereoisomer, solvate, prodrug, isotopic derivative, or tautomer thereof wherein all variables are as defined herein.

In some embodiments, the compound is of Formula (I-II-I-A-1-4-a):

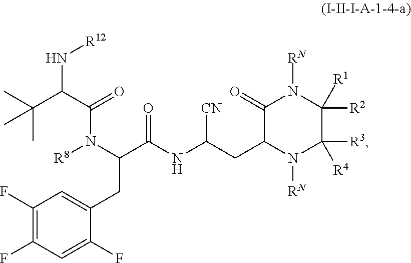

or a pharmaceutically acceptable salt, stereoisomer, solvate, prodrug, isotopic derivative, or tautomer thereof wherein all variables are as defined herein.

In some embodiments, the compound is of Formula (I-II-I-A-2-4-a):

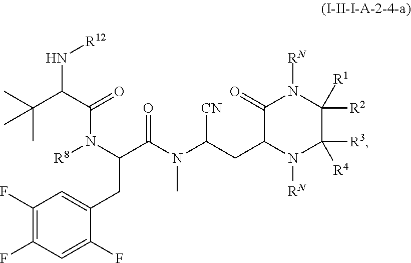

or a pharmaceutically acceptable salt, stereoisomer, solvate, prodrug, isotopic derivative, or tautomer thereof wherein all variables are as defined herein.

In some embodiments, the compound is of Formula (I-II-I-A-1-4-a*):

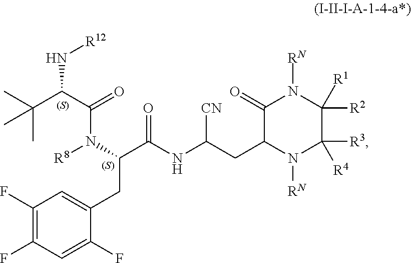

or a pharmaceutically acceptable salt, stereoisomer, solvate, prodrug, isotopic derivative, or tautomer thereof wherein all variables are as defined herein.

In some embodiments, the compound is of Formula (I-II-I-A-2-4-a*):

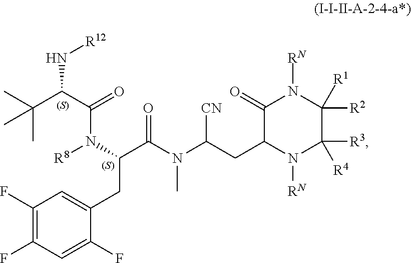

or a pharmaceutically acceptable salt, stereoisomer, solvate, prodrug, isotopic derivative, or tautomer thereof wherein all variables are as defined herein.

In some embodiments, the compound is of Formula (I-II-II-A):

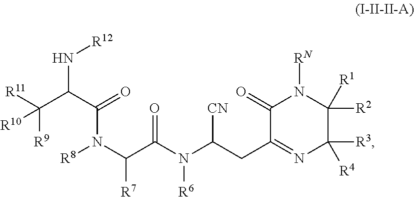

or a pharmaceutically acceptable salt, stereoisomer, solvate, prodrug, isotopic derivative, or tautomer thereof wherein all variables are as defined herein.

In some embodiments, the compound is of Formula (I-II-II-A-1):

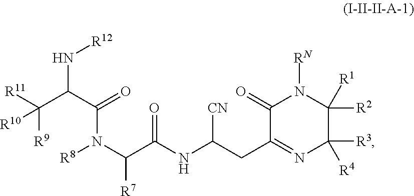

or a pharmaceutically acceptable salt, stereoisomer, solvate, prodrug, isotopic derivative, or tautomer thereof wherein all variables are as defined herein.

In some embodiments, the compound is of Formula (I-II-II-A-2):

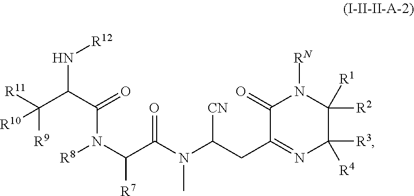

or a pharmaceutically acceptable salt, stereoisomer, solvate, prodrug, isotopic derivative, or tautomer thereof wherein all variables are as defined herein.

In some embodiments, the compound is of Formula (I-II-II-A-A):

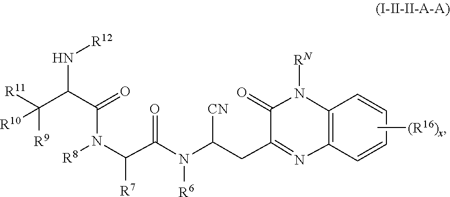

or a pharmaceutically acceptable salt, stereoisomer, solvate, prodrug, isotopic derivative, or tautomer thereof wherein x is an integer selected from 0, 1, 2, 3 and 4; and all other variables are as defined herein.

In some embodiments, the compound is of Formula (I-II-II-A-1-1):

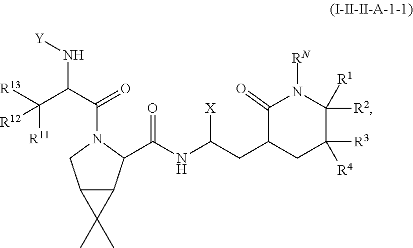

or a pharmaceutically acceptable salt, stereoisomer, solvate, prodrug, isotopic derivative, or tautomer thereof wherein all variables are as defined herein.

In some embodiments, the compound is of Formula (I-II-II-A-2-1):

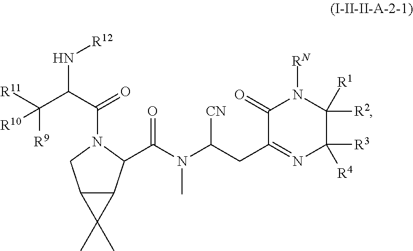

or a pharmaceutically acceptable salt, stereoisomer, solvate, prodrug, isotopic derivative, or tautomer thereof wherein all variables are as defined herein.

In some embodiments, the compound is of Formula (I-II-II-A-1-1-a):

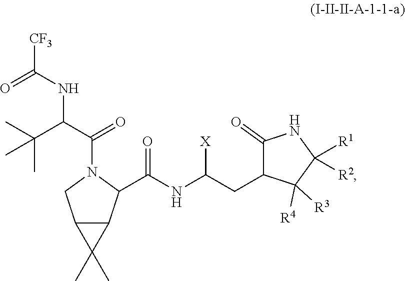

or a pharmaceutically acceptable salt, stereoisomer, solvate, prodrug, isotopic derivative, or tautomer thereof wherein all variables are as defined herein.

In some embodiments, the compound is of Formula (I-II-II-A-2-1-a):

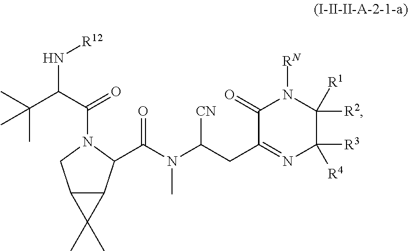

or a pharmaceutically acceptable salt, stereoisomer, solvate, prodrug, isotopic derivative, or tautomer thereof wherein all variables are as defined herein.

In some embodiments, the compound is of Formula (I-II-II-A-1-1-a*):

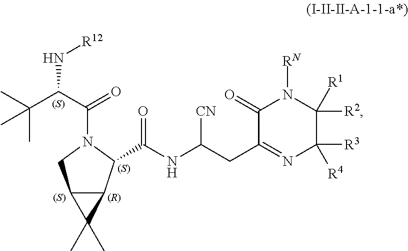

or a pharmaceutically acceptable salt, stereoisomer, solvate, prodrug, isotopic derivative, or tautomer thereof wherein all variables are as defined herein.

In some embodiments, the compound is of Formula (I-II-II-A-2-1-a*):

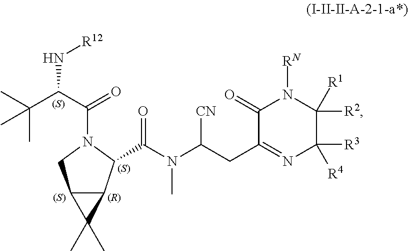

or a pharmaceutically acceptable salt, stereoisomer, solvate, prodrug, isotopic derivative, or tautomer thereof wherein all variables are as defined herein.

In some embodiments, the compound is of Formula (I-II-II-A-1-2):

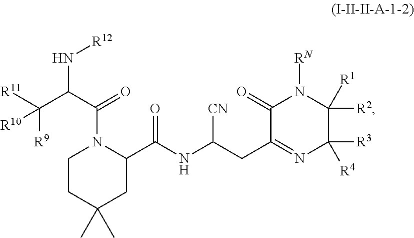

or a pharmaceutically acceptable salt, stereoisomer, solvate, prodrug, isotopic derivative, or tautomer thereof wherein all variables are as defined herein.

In some embodiments, the compound is of Formula (I-II-II-A-2-2):

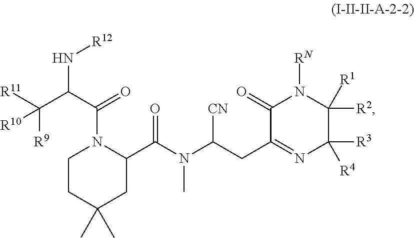

or a pharmaceutically acceptable salt, stereoisomer, solvate, prodrug, isotopic derivative, or tautomer thereof wherein all variables are as defined herein.

In some embodiments, the compound is of Formula (I-II-II-A-1-2-a):

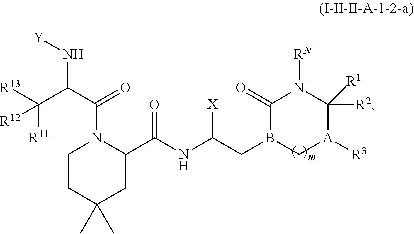

or a pharmaceutically acceptable salt, stereoisomer, solvate, prodrug, isotopic derivative, or tautomer thereof wherein all variables are as defined herein.

In some embodiments, the compound is of Formula (I-II-II-A-2-2-a):

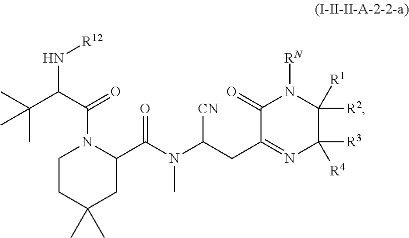

or a pharmaceutically acceptable salt, stereoisomer, solvate, prodrug, isotopic derivative, or tautomer thereof wherein all variables are as defined herein.

In some embodiments, the compound is of Formula (I-II-II-A-1-2-a*):

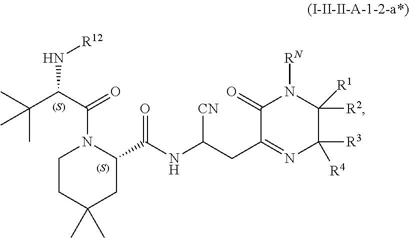

or a pharmaceutically acceptable salt, stereoisomer, solvate, prodrug, isotopic derivative, or tautomer thereof wherein all variables are as defined herein.

In some embodiments, the compound is of Formula (I-II-II-A-2-2-a*):

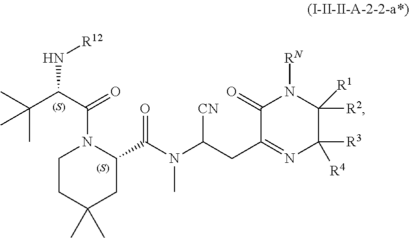

or a pharmaceutically acceptable salt, stereoisomer, solvate, prodrug, isotopic derivative, or tautomer thereof wherein all variables are as defined herein.

In some embodiments, the compound is of Formula (I-II-II-A-1-3):

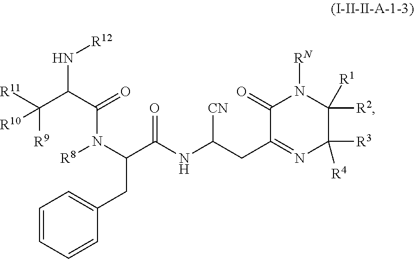

or a pharmaceutically acceptable salt, stereoisomer, solvate, prodrug, isotopic derivative, or tautomer thereof wherein all variables are as defined herein.

In some embodiments, the compound is of Formula (I-II-II-A-2-3):

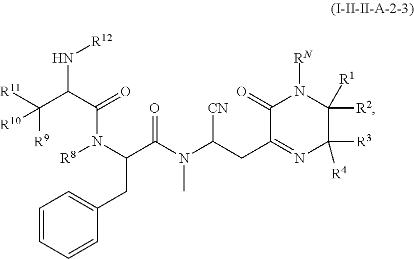

or a pharmaceutically acceptable salt, stereoisomer, solvate, prodrug, isotopic derivative, or tautomer thereof wherein all variables are as defined herein.

In some embodiments, the compound is of Formula (I-II-II-A-1-3-a):

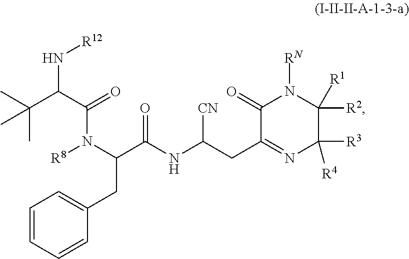

or a pharmaceutically acceptable salt, stereoisomer, solvate, prodrug, isotopic derivative, or tautomer thereof wherein all variables are as defined herein.

In some embodiments, the compound is of Formula (I-II-II-A-2-3-a):

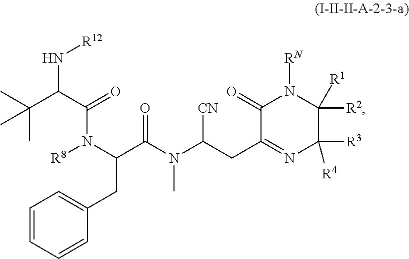

or a pharmaceutically acceptable salt, stereoisomer, solvate, prodrug, isotopic derivative, or tautomer thereof wherein all variables are as defined herein.

In some embodiments, the compound is of Formula (I-II-II-A-1-3-a*):

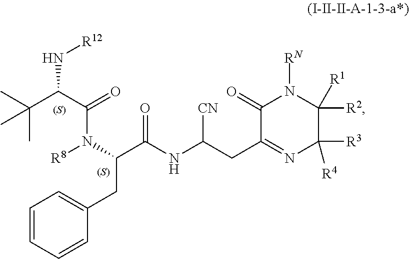

or a pharmaceutically acceptable salt, stereoisomer, solvate, prodrug, isotopic derivative, or tautomer thereof wherein all variables are as defined herein.

In some embodiments, the compound is of Formula (I-II-II-A-2-3-a*):

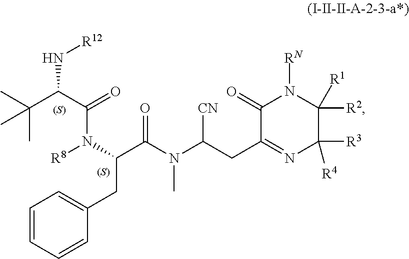

or a pharmaceutically acceptable salt, stereoisomer, solvate, prodrug, isotopic derivative, or tautomer thereof wherein all variables are as defined herein.

In some embodiments, the compound is of Formula (I-II-II-A-1-4):

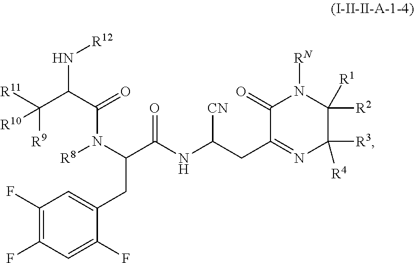

or a pharmaceutically acceptable salt, stereoisomer, solvate, prodrug, isotopic derivative, or tautomer thereof wherein all variables are as defined herein.

In some embodiments, the compound is of Formula (I-II-II-A-2-4):

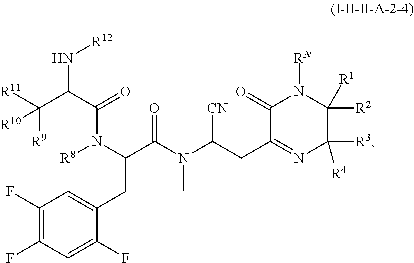

or a pharmaceutically acceptable salt, stereoisomer, solvate, prodrug, isotopic derivative, or tautomer thereof wherein all variables are as defined herein.

In some embodiments, the compound is of Formula (I-II-II-A-1-4-a):

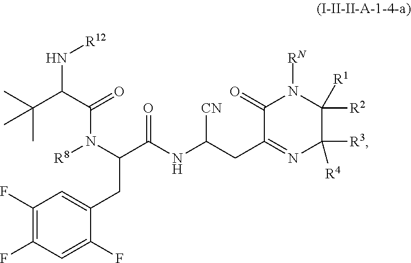

or a pharmaceutically acceptable salt, stereoisomer, solvate, prodrug, isotopic derivative, or tautomer thereof wherein all variables are as defined herein.

In some embodiments, the compound is of Formula (I-II-II-A-2-4-a):

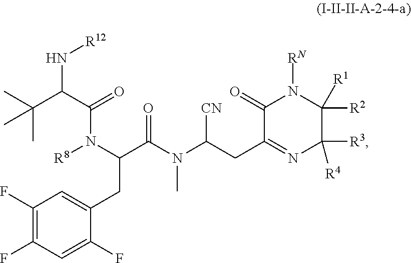

or a pharmaceutically acceptable salt, stereoisomer, solvate, prodrug, isotopic derivative, or tautomer thereof wherein all variables are as defined herein.

In some embodiments, the compound is of Formula (I-II-II-A-1-4-a*):

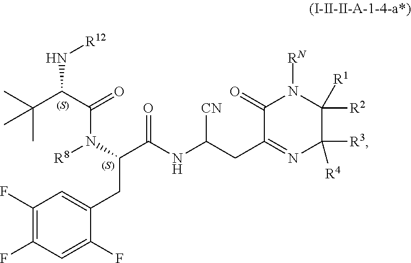

or a pharmaceutically acceptable salt, stereoisomer, solvate, prodrug, isotopic derivative, or tautomer thereof wherein all variables are as defined herein.

In some embodiments, the compound is of Formula (I-II-II-A-2-4-a*):

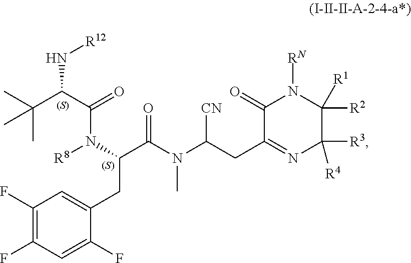

or a pharmaceutically acceptable salt, stereoisomer, solvate, prodrug, isotopic derivative, or tautomer thereof wherein all variables are as defined herein.

In some embodiments, the compound is of Formula (I-I-II-B):

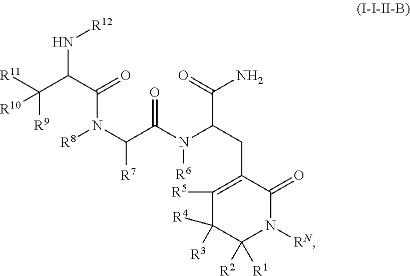

or a pharmaceutically acceptable salt, stereoisomer, solvate, prodrug, isotopic derivative, or tautomer thereof wherein all variables are as defined herein.

In some embodiments, the compound is of Formula (I-I-II-B-1):

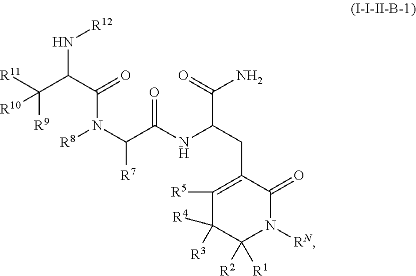

or a pharmaceutically acceptable salt, stereoisomer, solvate, prodrug, isotopic derivative, or tautomer thereof wherein all variables are as defined herein.

In some embodiments, the compound is of Formula (I-I-II-B-2):

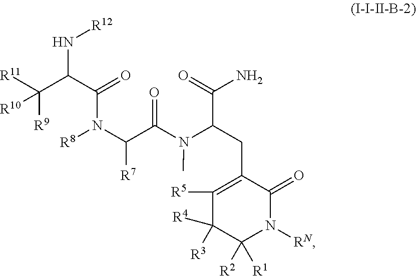

or a pharmaceutically acceptable salt, stereoisomer, solvate, prodrug, isotopic derivative, or tautomer thereof wherein all variables are as defined herein.

In some embodiments, the compound is of Formula (I-I-II-B-1-1):

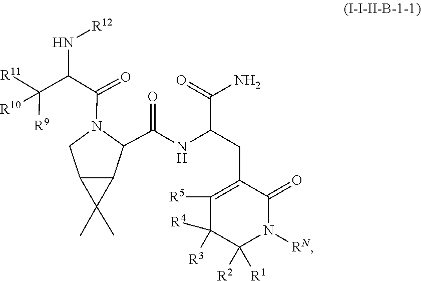

or a pharmaceutically acceptable salt, stereoisomer, solvate, prodrug, isotopic derivative, or tautomer thereof wherein all variables are as defined herein.

In some embodiments, the compound is of Formula (I-I-II-B-2-1):

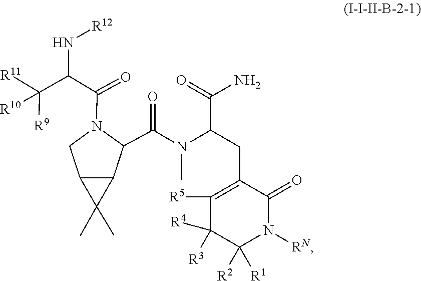

or a pharmaceutically acceptable salt, stereoisomer, solvate, prodrug, isotopic derivative, or tautomer thereof wherein all variables are as defined herein.

In some embodiments, the compound is of Formula (I-I-II-B-1-1-a):

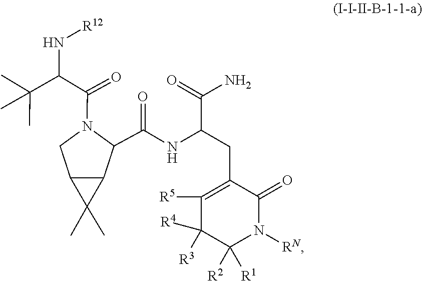

or a pharmaceutically acceptable salt, stereoisomer, solvate, prodrug, isotopic derivative, or tautomer thereof wherein all variables are as defined herein.

In some embodiments, the compound is of Formula (I-I-II-B-2-1-a):

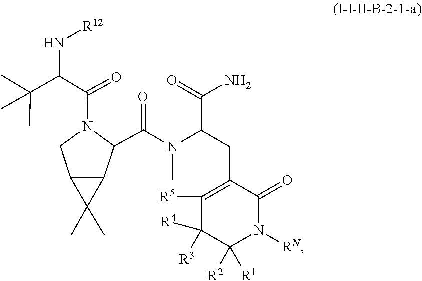

or a pharmaceutically acceptable salt, stereoisomer, solvate, prodrug, isotopic derivative, or tautomer thereof wherein all variables are as defined herein.

In some embodiments, the compound is of Formula (I-I-II-B-1-1-a*):

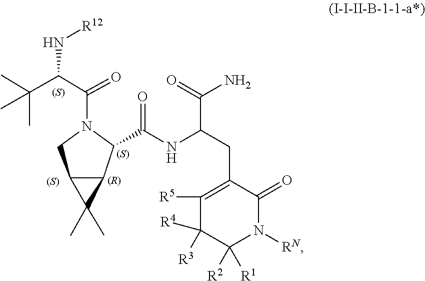

or a pharmaceutically acceptable salt, stereoisomer, solvate, prodrug, isotopic derivative, or tautomer thereof wherein all variables are as defined herein.

In some embodiments, the compound is of Formula (I-I-II-B-2-1-a*):

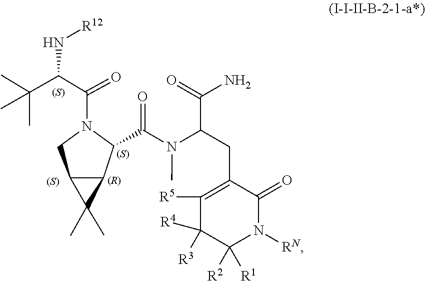

or a pharmaceutically acceptable salt, stereoisomer, solvate, prodrug, isotopic derivative, or tautomer thereof wherein all variables are as defined herein.

In some embodiments, the compound is of Formula (I-I-II-B-1-2):

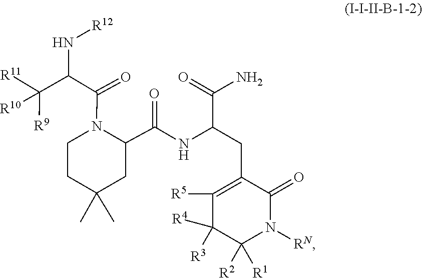

or a pharmaceutically acceptable salt, stereoisomer, solvate, prodrug, isotopic derivative, or tautomer thereof wherein all variables are as defined herein.

In some embodiments, the compound is of Formula (I-I-II-B-2-2):

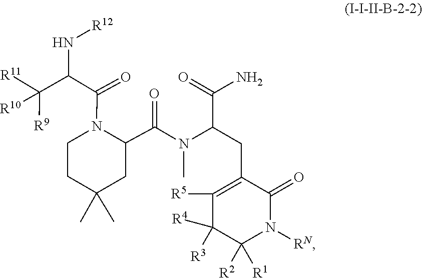

or a pharmaceutically acceptable salt, stereoisomer, solvate, prodrug, isotopic derivative, or tautomer thereof wherein all variables are as defined herein.

In some embodiments, the compound is of Formula (I-I-II-B-1-2-a):

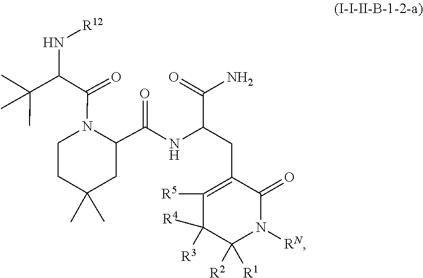

or a pharmaceutically acceptable salt, stereoisomer, solvate, prodrug, isotopic derivative, or tautomer thereof wherein all variables are as defined herein.

In some embodiments, the compound is of Formula (I-I-II-B-2-2-a):

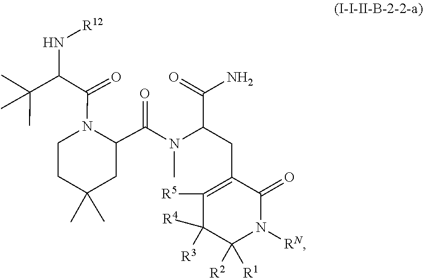

or a pharmaceutically acceptable salt, stereoisomer, solvate, prodrug, isotopic derivative, or tautomer thereof wherein all variables are as defined herein.

In some embodiments, the compound is of Formula (I-I-II-B-1-2-a*):

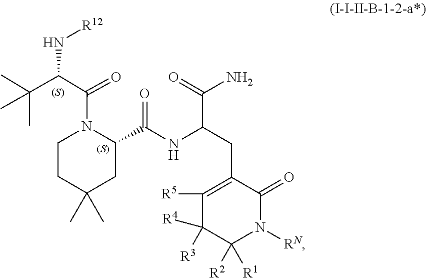

or a pharmaceutically acceptable salt, stereoisomer, solvate, prodrug, isotopic derivative, or tautomer thereof wherein all variables are as defined herein.

In some embodiments, the compound is of Formula (I-I-II-B-2-2-a*):

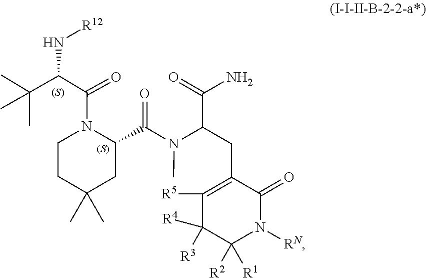

or a pharmaceutically acceptable salt, stereoisomer, solvate, prodrug, isotopic derivative, or tautomer thereof wherein all variables are as defined herein.

In some embodiments, the compound is of Formula (I-I-II-B-1-3):

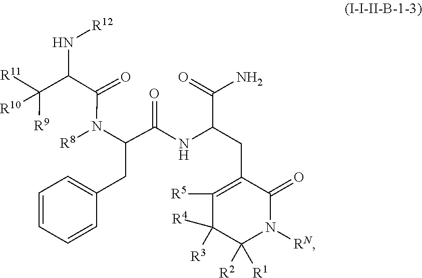

or a pharmaceutically acceptable salt, stereoisomer, solvate, prodrug, isotopic derivative, or tautomer thereof wherein all variables are as defined herein.

In some embodiments, the compound is of Formula (I-I-II-B-2-3):

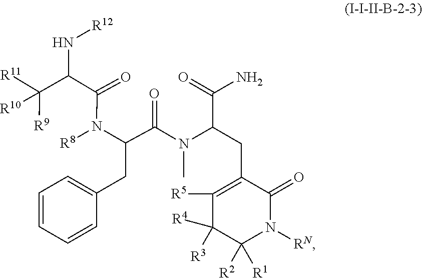

or a pharmaceutically acceptable salt, stereoisomer, solvate, prodrug, isotopic derivative, or tautomer thereof wherein all variables are as defined herein.

In some embodiments, the compound is of Formula (I-I-II-B-1-3-a):

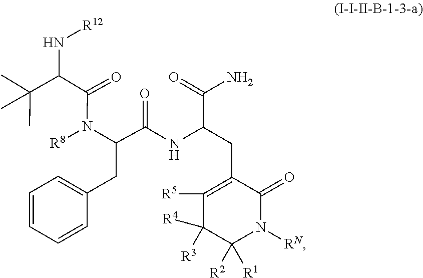

or a pharmaceutically acceptable salt, stereoisomer, solvate, prodrug, isotopic derivative, or tautomer thereof wherein all variables are as defined herein.

In some embodiments, the compound is of Formula (I-I-II-B-2-3-a):

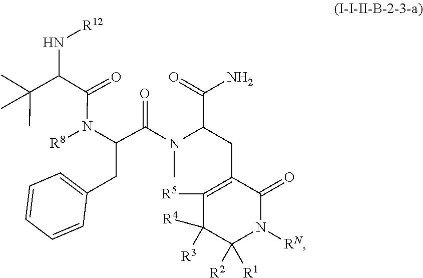

or a pharmaceutically acceptable salt, stereoisomer, solvate, prodrug, isotopic derivative, or tautomer thereof wherein all variables are as defined herein.

In some embodiments, the compound is of Formula (I-I-II-B-1-3-a*):

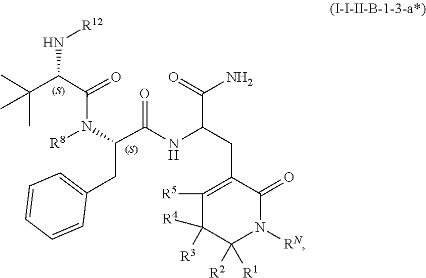

or a pharmaceutically acceptable salt, stereoisomer, solvate, prodrug, isotopic derivative, or tautomer thereof wherein all variables are as defined herein.

In some embodiments, the compound is of Formula (I-I-II-B-2-3-a*):

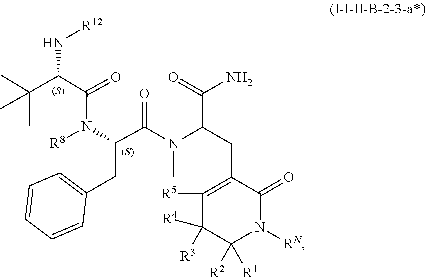

or a pharmaceutically acceptable salt, stereoisomer, solvate, prodrug, isotopic derivative, or tautomer thereof wherein all variables are as defined herein.

In some embodiments, the compound is of Formula (I-I-II-B-1-4):

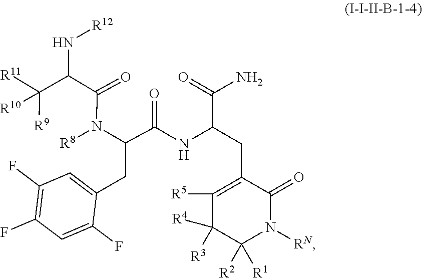

or a pharmaceutically acceptable salt, stereoisomer, solvate, prodrug, isotopic derivative, or tautomer thereof wherein all variables are as defined herein.

In some embodiments, the compound is of Formula (I-I-II-B-2-4):

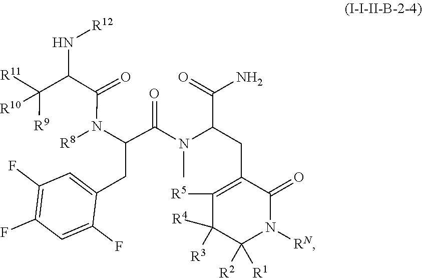

or a pharmaceutically acceptable salt, stereoisomer, solvate, prodrug, isotopic derivative, or tautomer thereof wherein all variables are as defined herein.

In some embodiments, the compound is of Formula (I-I-II-B-1-4-a):

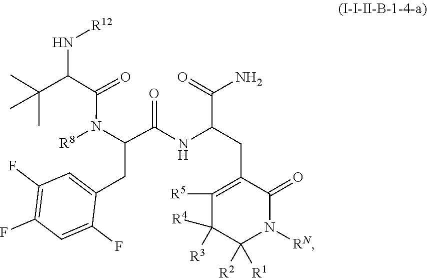

or a pharmaceutically acceptable salt, stereoisomer, solvate, prodrug, isotopic derivative, or tautomer thereof wherein all variables are as defined herein.

In some embodiments, the compound is of Formula (I-I-II-B-2-4-a):

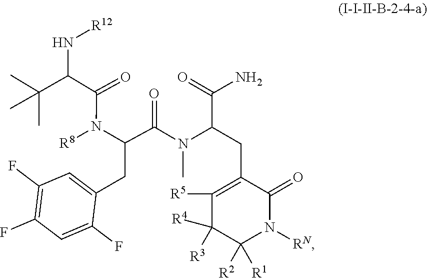

or a pharmaceutically acceptable salt, stereoisomer, solvate, prodrug, isotopic derivative, or tautomer thereof wherein all variables are as defined herein.

In some embodiments, the compound is of Formula (I-I-II-B-1-4-a*):

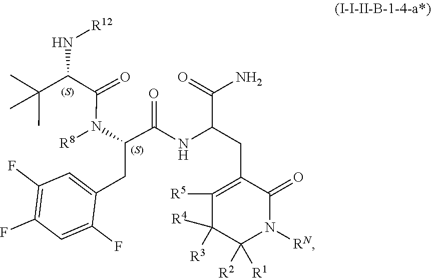

or a pharmaceutically acceptable salt, stereoisomer, solvate, prodrug, isotopic derivative, or tautomer thereof wherein all variables are as defined herein.

In some embodiments, the compound is of Formula (I-I-II-B-2-4-a*):

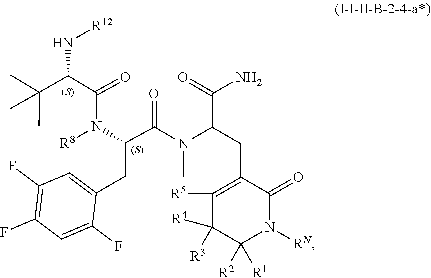

or a pharmaceutically acceptable salt, stereoisomer, solvate, prodrug, isotopic derivative, or tautomer thereof wherein all variables are as defined herein.

In some embodiments, the compound is of Formula (I-G-A):

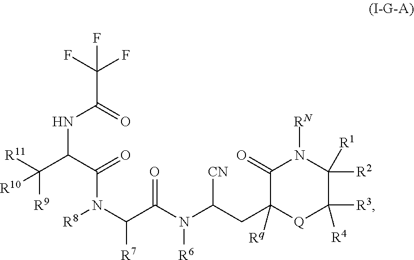

or a pharmaceutically acceptable salt, stereoisomer, solvate, prodrug, isotopic derivative, or tautomer thereof.

In some embodiments, the compound is of Formula (I-G-B-G):

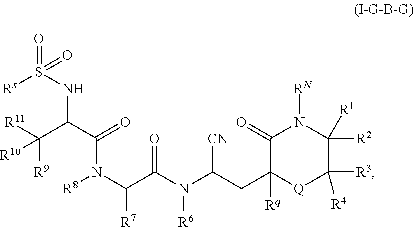

or a pharmaceutically acceptable salt, stereoisomer, solvate, prodrug, isotopic derivative, or tautomer thereof, wherein R^{s} is C₁-C₆ alkyl, or C₃-C₇ cycloalkyl is optionally substituted with one or more halogen.

In some embodiments, the compound is of Formula (I-G-B):

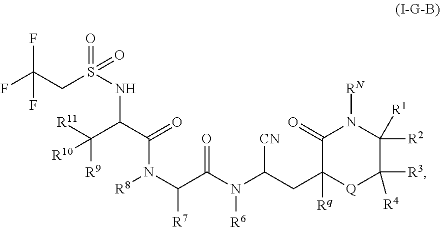

or a pharmaceutically acceptable salt, stereoisomer, solvate, prodrug, isotopic derivative, or tautomer thereof.

In some embodiments, the compound is of Formula (I-G-C):

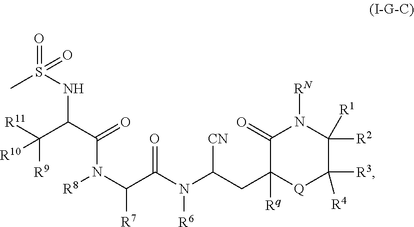

or a pharmaceutically acceptable salt, stereoisomer, solvate, prodrug, isotopic derivative, or tautomer thereof.

In some embodiments, the compound is of Formula (I-A-A-D-G):

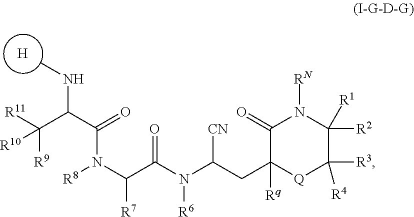

or a pharmaceutically acceptable salt, stereoisomer, solvate, prodrug, isotopic derivative, or tautomer thereof, wherein H is a 5-6 membered heteroaryl, comprising 1-3 heteroatom selected from O, N, S optionally substituted with one or more R¹⁶.

In some embodiments, the compound is of Formula (I-G-D):

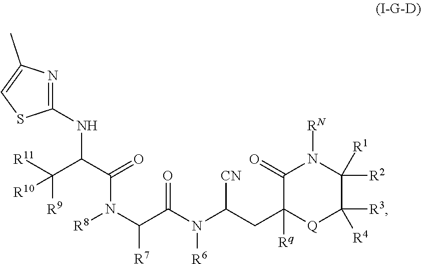

or a pharmaceutically acceptable salt, stereoisomer, solvate, prodrug, isotopic derivative, or tautomer thereof.

In some embodiments, the compound is of Formula (I-G-E):

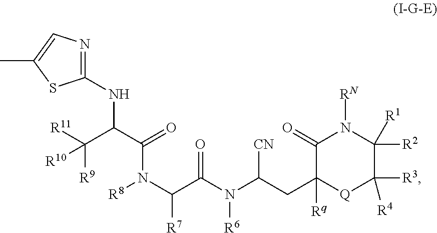

or a pharmaceutically acceptable salt, stereoisomer, solvate, prodrug, isotopic derivative, or tautomer thereof.

In some embodiments, the compound is of Formula (I-G-F):

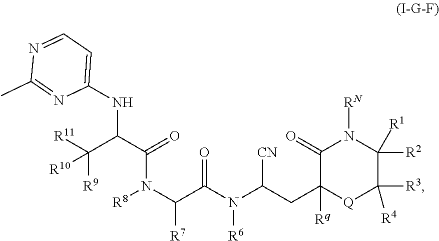

or a pharmaceutically acceptable salt, stereoisomer, solvate, prodrug, isotopic derivative, or tautomer thereof.

In some embodiments, the compound is of Formula (I-G-G):

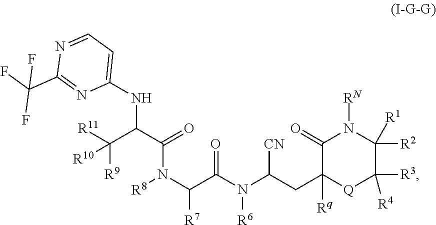

or a pharmaceutically acceptable salt, stereoisomer, solvate, prodrug, isotopic derivative, or tautomer thereof.

In some embodiments, the compound is of Formula (I-G-H):

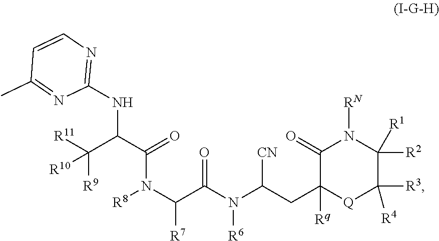

or a pharmaceutically acceptable salt, stereoisomer, solvate, prodrug, isotopic derivative, or tautomer thereof.

In some embodiments, the compound is of Formula (I-G-J):

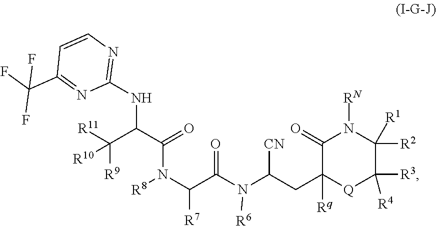

or a pharmaceutically acceptable salt, stereoisomer, solvate, prodrug, isotopic derivative, or tautomer thereof.

In some embodiments, the compound is of Formula (I-G-K):

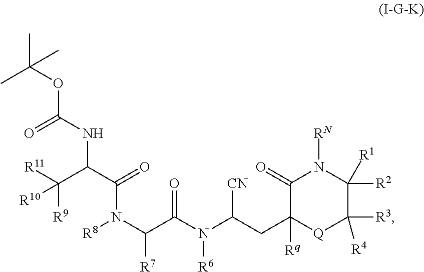

or a pharmaceutically acceptable salt, stereoisomer, solvate, prodrug, isotopic derivative, or tautomer thereof.

In some embodiments, the compound is of Formula (I-A-A-A):

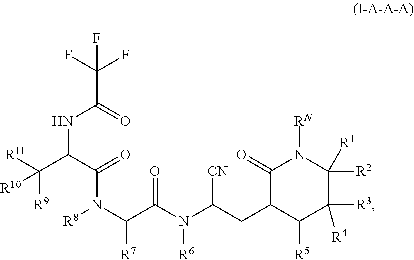

or a pharmaceutically acceptable salt, stereoisomer, solvate, prodrug, isotopic derivative, or tautomer thereof.

In some embodiments, the compound is of Formula (I-A-A-B-G):

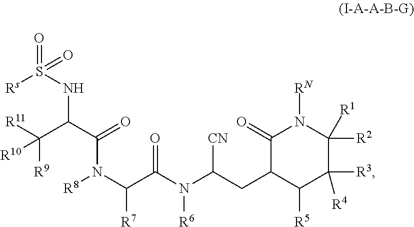

or a pharmaceutically acceptable salt, stereoisomer, solvate, prodrug, isotopic derivative, or tautomer thereof, wherein R^{s} is C₁-C₆ alkyl is optionally substituted with one or more halogen.

In some embodiments, the compound is of Formula (I-A-A-B):

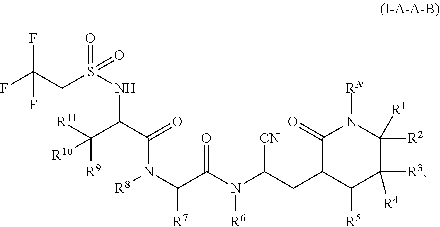

or a pharmaceutically acceptable salt, stereoisomer, solvate, prodrug, isotopic derivative, or tautomer thereof.

In some embodiments, the compound is of Formula (I-A-A-C):

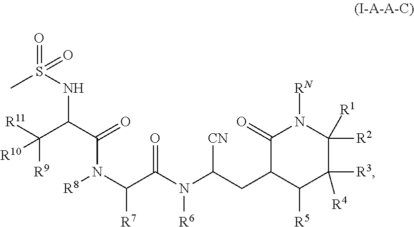

or a pharmaceutically acceptable salt, stereoisomer, solvate, prodrug, isotopic derivative, or tautomer thereof.

In some embodiments, the compound is of Formula (I-A-A-D-G):

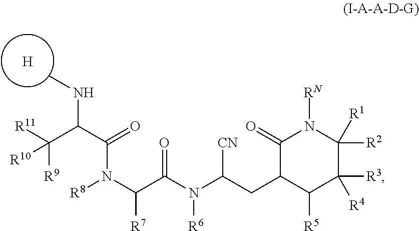

or a pharmaceutically acceptable salt, stereoisomer, solvate, prodrug, isotopic derivative, or tautomer thereof, wherein H is a 5-6 membered heteroaryl, comprising 1-3 heteroatom selected from O, N, S optionally substituted with one or more R¹⁶.

In some embodiments, the compound is of Formula (I-A-A-D):

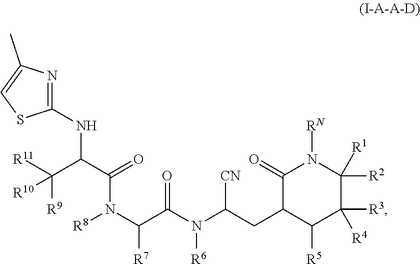

or a pharmaceutically acceptable salt, stereoisomer, solvate, prodrug, isotopic derivative, or tautomer thereof.

In some embodiments, the compound is of Formula (I-A-A-E):

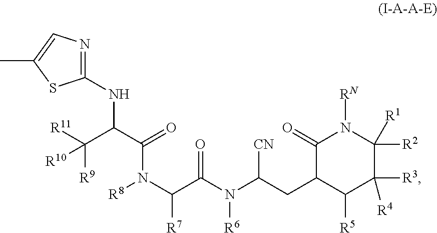

or a pharmaceutically acceptable salt, stereoisomer, solvate, prodrug, isotopic derivative, or tautomer thereof.

In some embodiments, the compound is of Formula (I-A-A-F):

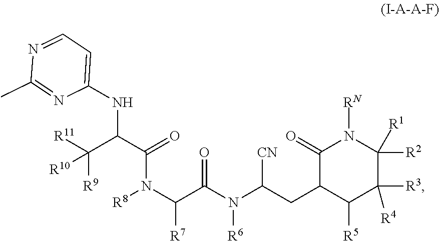

or a pharmaceutically acceptable salt, stereoisomer, solvate, prodrug, isotopic derivative, or tautomer thereof.

In some embodiments, the compound is of Formula (I-A-A-G):

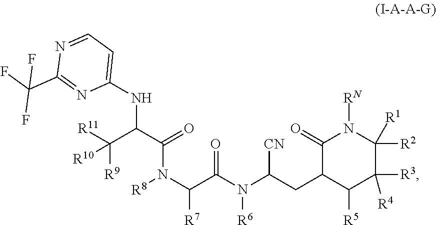

or a pharmaceutically acceptable salt, stereoisomer, solvate, prodrug, isotopic derivative, or tautomer thereof.

In some embodiments, the compound is of Formula (I-A-A-H):

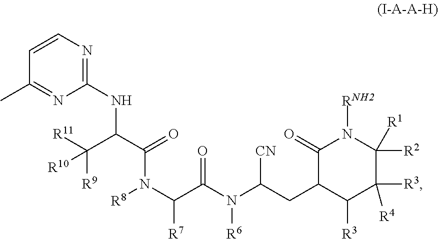

or a pharmaceutically acceptable salt, stereoisomer, solvate, prodrug, isotopic derivative, or tautomer thereof.

In some embodiments, the compound is of Formula (I-A-A-J):

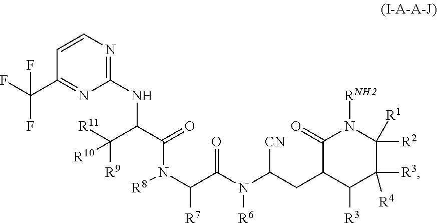

or a pharmaceutically acceptable salt, stereoisomer, solvate, prodrug, isotopic derivative, or tautomer thereof.

In some embodiments, the compound is of Formula (I-A-A-K):

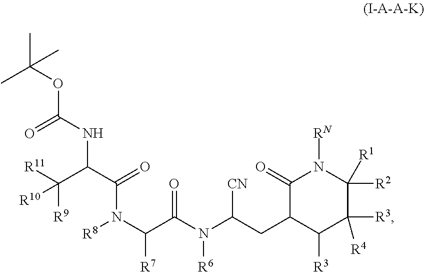

or a pharmaceutically acceptable salt, stereoisomer, solvate, prodrug, isotopic derivative, or tautomer thereof.

In some embodiments, the compound is of Formula (I-I-II-A-A):

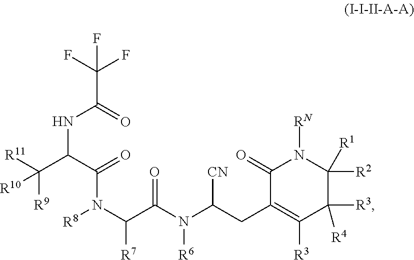

or a pharmaceutically acceptable salt, stereoisomer, solvate, prodrug, isotopic derivative, or tautomer thereof.

In some embodiments, the compound is of Formula (I-I-II-A-B-G):

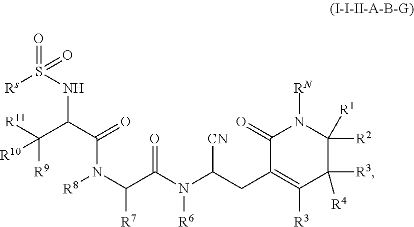

or a pharmaceutically acceptable salt, stereoisomer, solvate, prodrug, isotopic derivative, or tautomer thereof, wherein R^{s} is C₁-C₆ alkyl is optionally substituted with one or more halogen.

In some embodiments, the compound is of Formula (I-I-II-A-B-G-1):

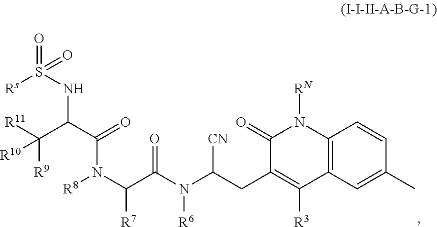

or a pharmaceutically acceptable salt, stereoisomer, solvate, prodrug, isotopic derivative, or tautomer thereof, wherein R^{s} is C₁-C₆ alkyl is optionally substituted with one or more halogen.

In some embodiments, the compound is of Formula (I-I-II-A-B-G-2):

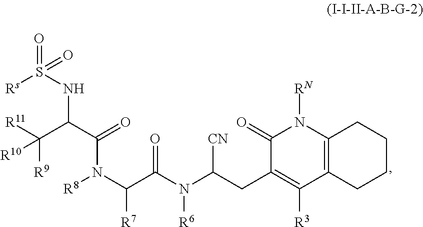

or a pharmaceutically acceptable salt, stereoisomer, solvate, prodrug, isotopic derivative, or tautomer thereof, wherein R^{s} is C₁-C₆ alkyl is optionally substituted with one or more halogen.

In some embodiments, the compound is of Formula (I-I-II-A-B-G-3):

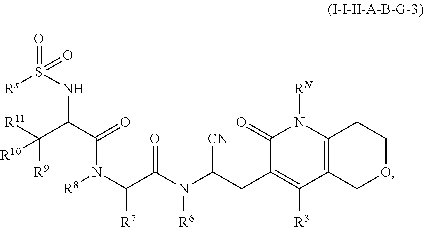

or a pharmaceutically acceptable salt, stereoisomer, solvate, prodrug, isotopic derivative, or tautomer thereof, wherein R^{s} is C₁-C₆ alkyl is optionally substituted with one or more halogen.

In some embodiments, the compound is of Formula (I-I-II-A-B-G-4):

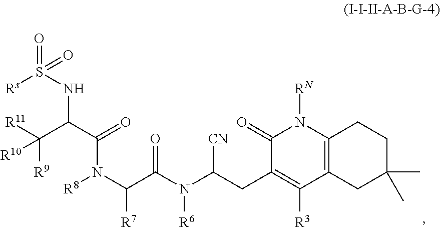

or a pharmaceutically acceptable salt, stereoisomer, solvate, prodrug, isotopic derivative, or tautomer thereof, wherein R^{s} is C₁-C₆ alkyl is optionally substituted with one or more halogen.

In some embodiments, the compound is of Formula (I-I-II-A-B-G-5):

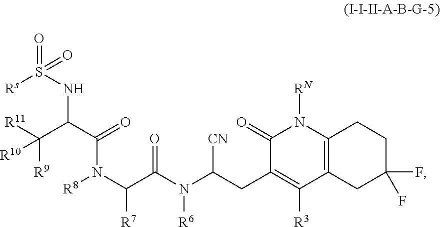

or a pharmaceutically acceptable salt, stereoisomer, solvate, prodrug, isotopic derivative, or tautomer thereof, wherein R^{s} is C₁-C₆ alkyl is optionally substituted with one or more halogen.

In some embodiments, the compound is of Formula (I-I-II-A-B-G-6):

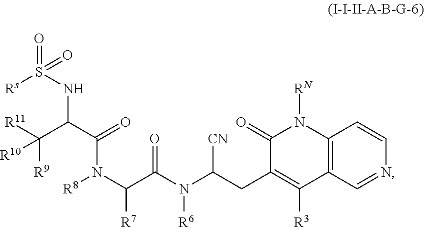

or a pharmaceutically acceptable salt, stereoisomer, solvate, prodrug, isotopic derivative, or tautomer thereof, wherein R^{s} is C₁-C₆ alkyl is optionally substituted with one or more halogen.

In some embodiments, the compound is of Formula (I-I-II-A-B-G-7):

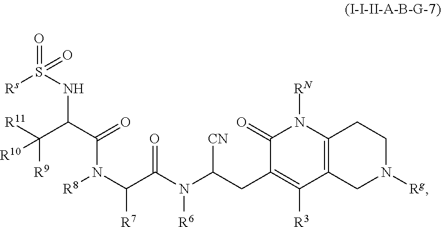

or a pharmaceutically acceptable salt, stereoisomer, solvate, prodrug, isotopic derivative, or tautomer thereof, wherein R^{s} is C₁-C₆ alkyl is optionally substituted with one or more halogen, R⁹ is selected from H, C₁-C₆ alkyl, —C(O)—C₁-C₆ alkyl.

In some embodiments, the compound is of Formula (I-I-II-A-B-G-8):

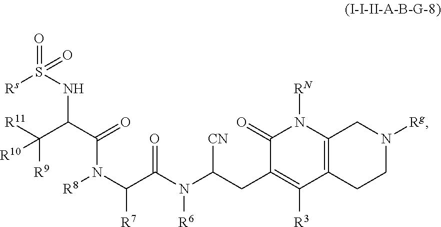

or a pharmaceutically acceptable salt, stereoisomer, solvate, prodrug, isotopic derivative, or tautomer thereof, wherein R^{s} is C₁-C₆ alkyl is optionally substituted with one or more halogen, R⁹ is selected from H, C₁-C₆ alkyl, —C(O)—C₁-C₆ alkyl.

In some embodiments, the compound is of Formula (I-I-II-A-B-1):

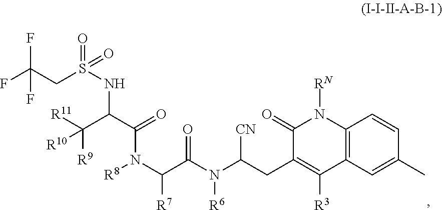

or a pharmaceutically acceptable salt, stereoisomer, solvate, prodrug, isotopic derivative, or tautomer thereof.

In some embodiments, the compound is of Formula (I-I-II-A-C):

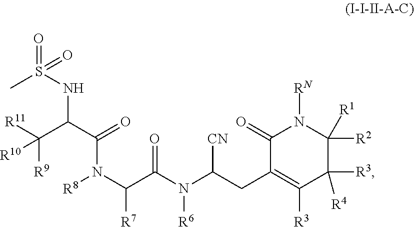

or a pharmaceutically acceptable salt, stereoisomer, solvate, prodrug, isotopic derivative, or tautomer thereof.

In some embodiments, the compound is of Formula (I-I-II-A-C-1):

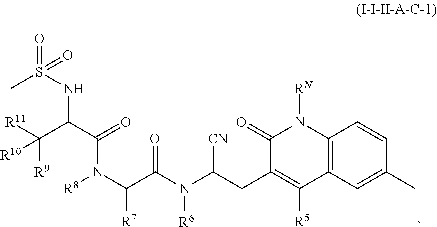

or a pharmaceutically acceptable salt, stereoisomer, solvate, prodrug, isotopic derivative, or tautomer thereof.

In some embodiments, the compound is of Formula (I-I-II-A-D-G):

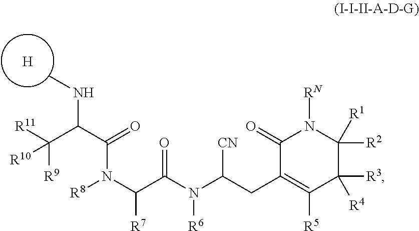

or a pharmaceutically acceptable salt, stereoisomer, solvate, prodrug, isotopic derivative, or tautomer thereof, wherein H is a 5-6 membered heteroaryl, comprising 1-3 heteroatom selected from O, N, S optionally substituted with one or more R¹⁶.

In some embodiments, the compound is of Formula (I-I-II-A-D-G-1):

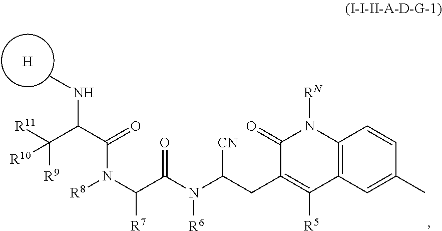

or a pharmaceutically acceptable salt, stereoisomer, solvate, prodrug, isotopic derivative, or tautomer thereof, wherein H is a 5-6 membered heteroaryl, comprising 1-3 heteroatom selected from O, N, S optionally substituted with one or more R¹⁶.

In some embodiments, the compound is of Formula (I-I-II-A-D):

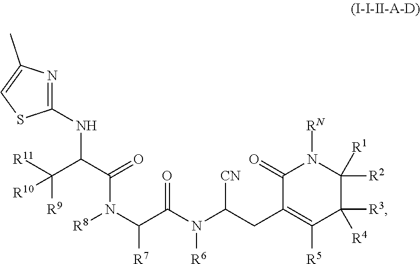

or a pharmaceutically acceptable salt, stereoisomer, solvate, prodrug, isotopic derivative, or tautomer thereof.

In some embodiments, the compound is of Formula (I-I-II-A-D-1):

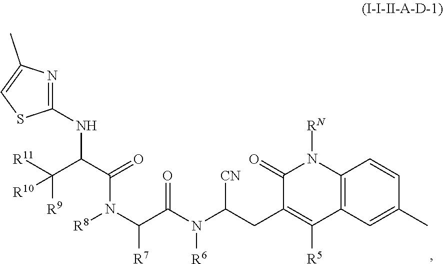

or a pharmaceutically acceptable salt, stereoisomer, solvate, prodrug, isotopic derivative, or tautomer thereof.

In some embodiments, the compound is of Formula (I-I-II-A-E):

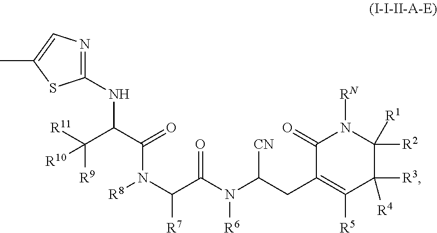

or a pharmaceutically acceptable salt, stereoisomer, solvate, prodrug, isotopic derivative, or tautomer thereof.

In some embodiments, the compound is of Formula (I-I-II-A-E-1):

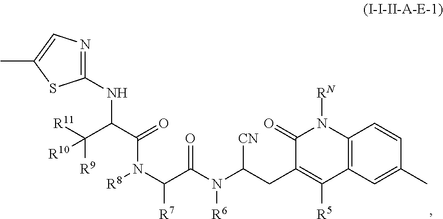

or a pharmaceutically acceptable salt, stereoisomer, solvate, prodrug, isotopic derivative, or tautomer thereof.

In some embodiments, the compound is of Formula (I-I-II-A-F):

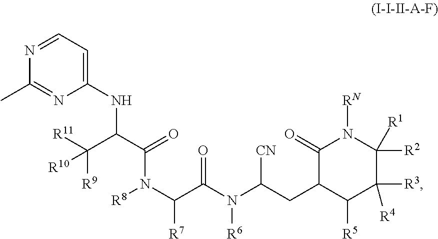

or a pharmaceutically acceptable salt, stereoisomer, solvate, prodrug, isotopic derivative, or tautomer thereof.

In some embodiments, the compound is of Formula (I-I-II-A-F-1):

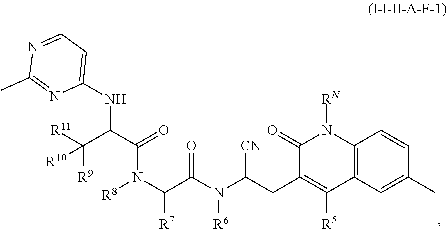

or a pharmaceutically acceptable salt, stereoisomer, solvate, prodrug, isotopic derivative, or tautomer thereof.

In some embodiments, the compound is of Formula (I-I-II-A-G):

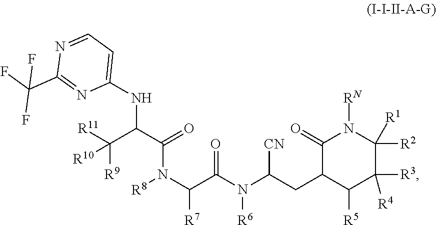

or a pharmaceutically acceptable salt, stereoisomer, solvate, prodrug, isotopic derivative, or tautomer thereof.

In some embodiments, the compound is of Formula (I-I-II-A-G-1):

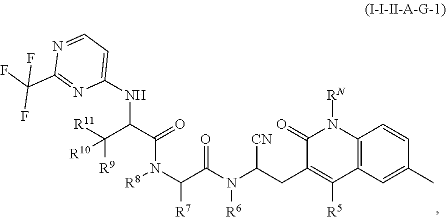

or a pharmaceutically acceptable salt, stereoisomer, solvate, prodrug, isotopic derivative, or tautomer thereof.

In some embodiments, the compound is of Formula (I-I-II-A-H):

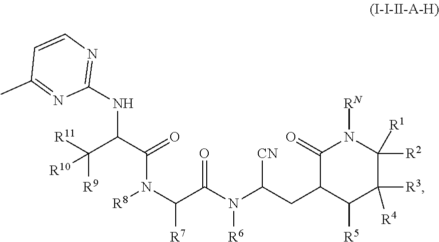

or a pharmaceutically acceptable salt, stereoisomer, solvate, prodrug, isotopic derivative, or tautomer thereof.

In some embodiments, the compound is of Formula (I-I-II-A-H-1):

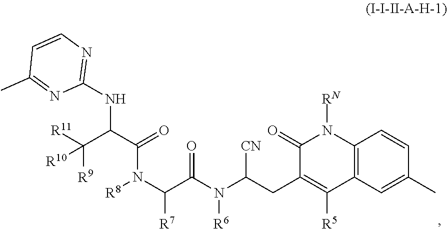

or a pharmaceutically acceptable salt, stereoisomer, solvate, prodrug, isotopic derivative, or tautomer thereof.

In some embodiments, the compound is of Formula (I-I-II-A-J):

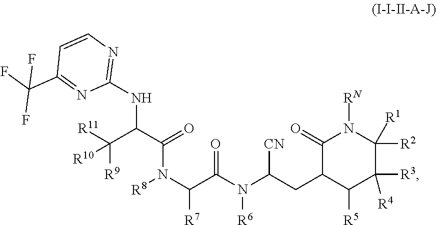

or a pharmaceutically acceptable salt, stereoisomer, solvate, prodrug, isotopic derivative, or tautomer thereof.

In some embodiments, the compound is of Formula (I-I-II-A-J-1):

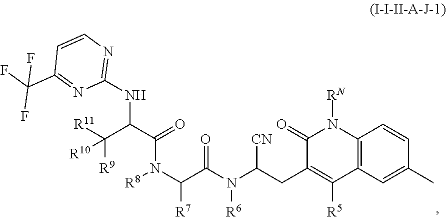

or a pharmaceutically acceptable salt, stereoisomer, solvate, prodrug, isotopic derivative, or tautomer thereof.

In some embodiments, the compound is of Formula (I-I-II-A-K):

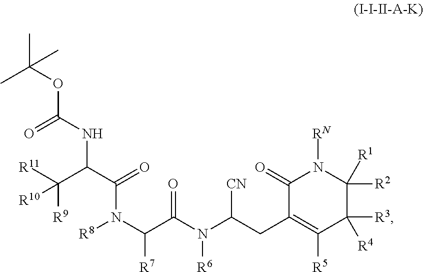

or a pharmaceutically acceptable salt, stereoisomer, solvate, prodrug, isotopic derivative, or tautomer thereof.

In some embodiments, the compound is of Formula (I-I-II-A-K-1):

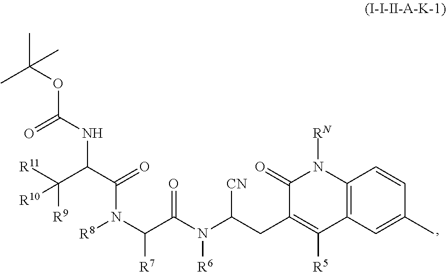

or a pharmaceutically acceptable salt, stereoisomer, solvate, prodrug, isotopic derivative, or tautomer thereof.

In some embodiments, the compound of Formula (I) is any combination of fragments (1), (2) and (3), presented in the Table 1.

TABLE 1
Lists of fragments (1), (2), and (3) that give the structure of the compound
of Formula (I) by combining any of fragment (1) with any of fragment (2) and any of fragment
(3).
(I)

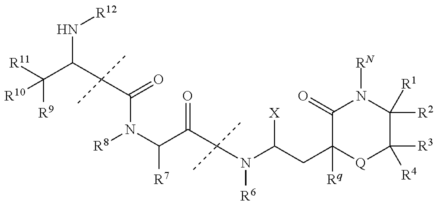

| | | |
|---|---|---|
| 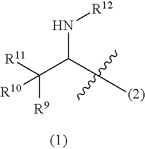 | 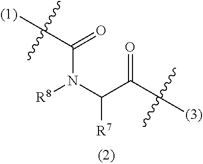 | 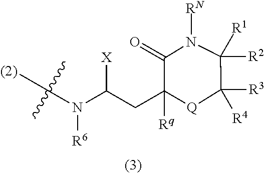 |

(1)

| | | |
|---|---|---|
| 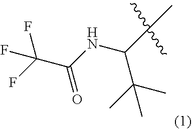 [SMILES: *C(NC(=O)C(F)(F)F)C(C)(C)C] | 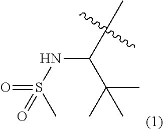 [SMILES: *C(NS(C)(=O)=O)C(C)(C)C] | 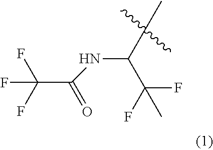 [SMILES: *C(NC(=O)C(F)(F)F)C(C)(F)F] |
| 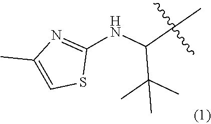 [SMILES: *C(Nc1nc(C)cs1)C(C)(C)C] | 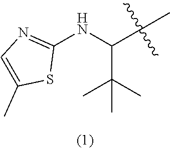 [SMILES: *C(Nc1ncc(C)s1)C(C)(C)C] | 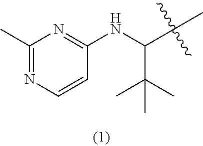 [SMILES: *C(Nc1ccnc(C)n1)C(C)(C)C] |
| 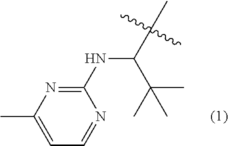 [SMILES: *C(Nc1nccc(C)n1)C(C)(C)C] | 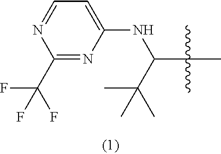 [SMILES: *C(Nc1ccnc(C(F)(F)F)n1)C(C)(C)C] | 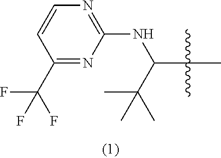 [SMILES: *C(Nc1nccc(C(F)(F)F)n1)C(C)(C)C] |
| 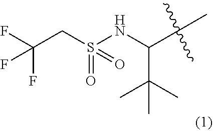 [SMILES: *C(NS(=O)(=O)CC(F)(F)F)C(C)(C)C] | 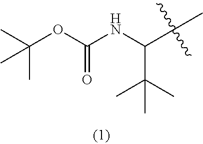 [SMILES: *C(NC(=O)OC(C)(C)C)C(C)(C)C] | 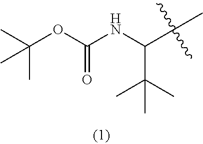 [SMILES: *C(NC(=O)OC(C)(C)C)C(C)(C)C] |
| 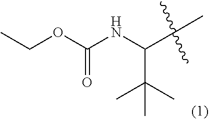 [SMILES: *C(NC(=O)OCC)C(C)(C)C] | 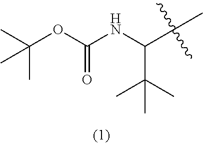 [SMILES: *C(NC(=O)OC(C)(C)C)C(C)(C)C] | 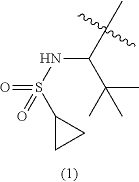 [SMILES: *C(NS(=O)(=O)C1CC1)C(C)(C)C] |

| | |
|---|---|
| 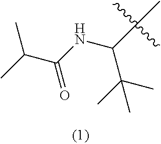 [SMILES: *C(NC(=O)C(C)C)C(C)(C)C] | 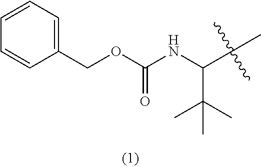 [SMILES: *C(NC(=O)OCc1ccccc1)C(C)(C)C] |

(2)

| | | |
|---|---|---|
| 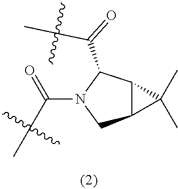 [SMILES: *C(=O)[C@@H]1[C@H]2[C@H](CN1C(*)=O)C2(C)C] | 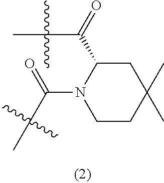 [SMILES: *C(=O)[C@@H]1CC(C)(C)CCN1C(*)=O] | 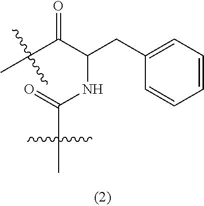 [SMILES: *C(=O)NC(Cc1ccccc1)C(*)=O] |
| 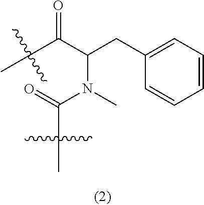 [SMILES: *C(=O)C(Cc1ccccc1)N(C)C(*)=O] | 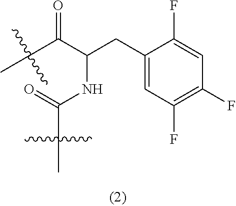 [SMILES: *C(=O)NC(Cc1cc(F)c(F)cc1F)C(*)=O] | 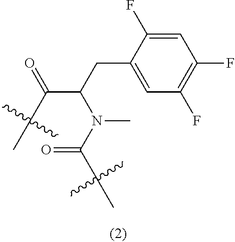 [SMILES: *C(=O)C(Cc1cc(F)c(F)cc1F)N(C)C(*)=O] |

| | |
|---|---|
| 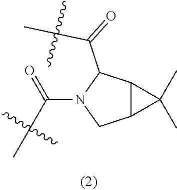 [SMILES: *C(=O)C1C2C(CN1C(*)=O)C2(C)C] | 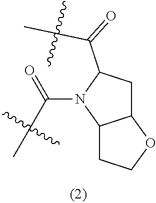 [SMILES: *C(=O)C1CC2OCCC2N1C(*)=O] |

(3)

| | | |
|---|---|---|
| 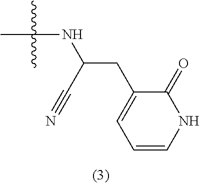 [SMILES: *NC(C#N)Cc1ccc[nH]c1=O] | 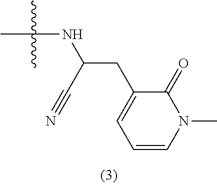 [SMILES: *NC(C#N)Cc1cccn(C)c1=O] | 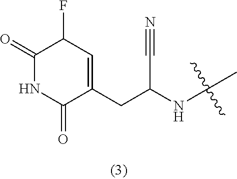 [SMILES: *NC(C#N)CC1=CC(F)C(=O)NC1=O] |
| 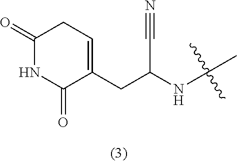 [SMILES: *NC(C#N)CC1=CCC(=O)NC1=O] | 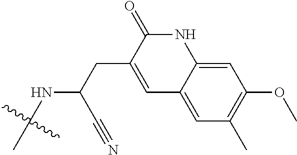 | 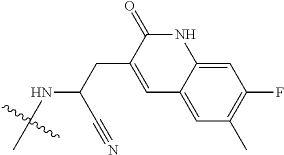 |
| | (3) | (3) |
| 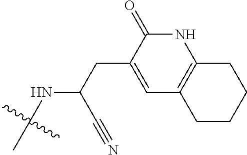 [SMILES: *NC(C#N)Cc1cc2c([nH]c1=O)CCCC2] | 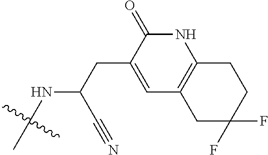 [SMILES: *NC(C#N)Cc1cc2c([nH]c1=O)CCC(F)(F)C2] | 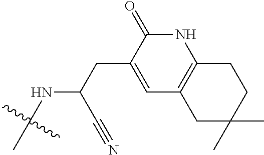 [SMILES: *NC(C#N)Cc1cc2c([nH]c1=O)CCC(C)(C)C2] |
| (3) | (3) | (3) |
| 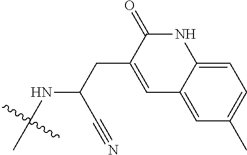 [SMILES: *NC(C#N)Cc1cc2cc(C)ccc2[nH]c1=O] | 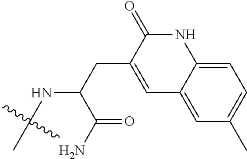 [SMILES: *NC(Cc1cc2cc(C)ccc2[nH]c1=O)C(N)=O] | 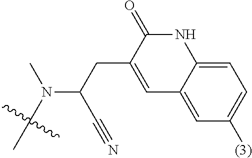 |
| (3) | (3) | (3) |
| 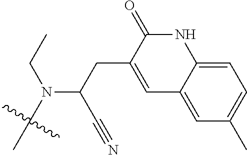 [SMILES: *N(CC)C(C#N)Cc1cc2cc(C)ccc2[nH]c1=O] | 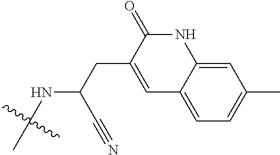 [SMILES: *NC(C#N)Cc1cc2ccc(C)cc2[nH]c1=O] | 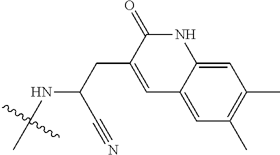 |
| (3) | (3) | (3) |
| 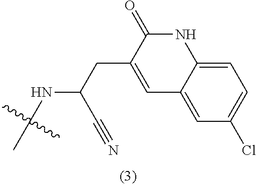 [SMILES: *NC(C#N)Cc1cc2cc(Cl)ccc2[nH]c1=O] | 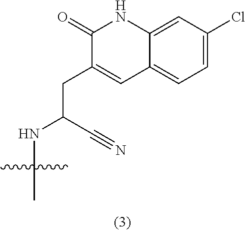 [SMILES: *NC(C#N)Cc1cc2ccc(Cl)cc2[nH]c1=O] | 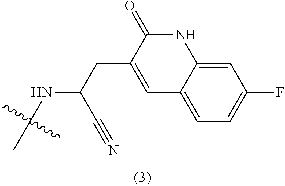 |
| 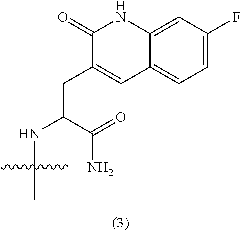 [SMILES: *NC(Cc1cc2ccc(F)cc2[nH]c1=O)C(N)=O] | 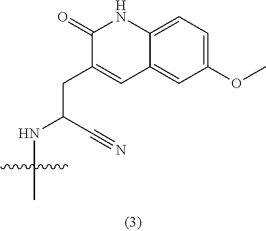 [SMILES: *NC(C#N)Cc1cc2cc(OC)ccc2[nH]c1=O] | 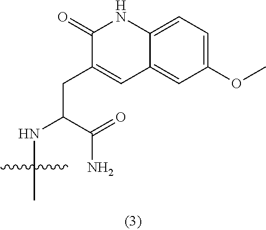 [SMILES: *NC(Cc1cc2cc(OC)ccc2[nH]c1=O)C(N)=O] |
| 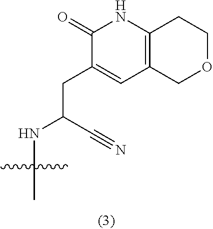 [SMILES: *NC(C#N)Cc1cc2c([nH]c1=O)CCOC2] | 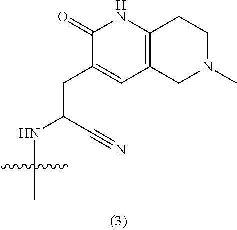 [SMILES: *NC(C#N)Cc1cc2c([nH]c1=O)CCN(C)C2] | 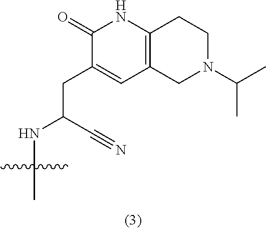 [SMILES: *NC(C#N)Cc1cc2c([nH]c1=O)CCN(C(C)C)C2] |
| 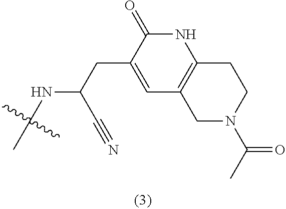 | 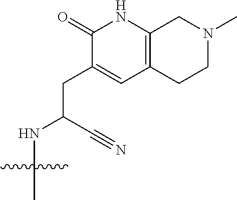 [SMILES: *NC(C#N)Cc1cc2c([nH]c1=O)CN(C)CC2] | 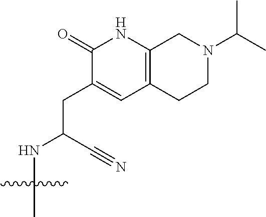 [SMILES: *NC(C#N)Cc1cc2c([nH]c1=O)CN(C(C)C)CC2] |
| | (3) | (3) |
| 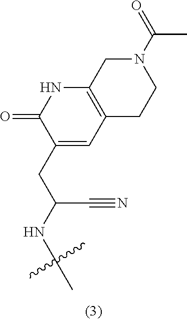 [SMILES: *NC(C#N)Cc1cc2c([nH]c1=O)CN(C(C)=O)CC2] | 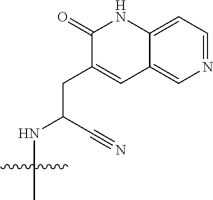 [SMILES: *NC(C#N)Cc1cc2cnccc2[nH]c1=O] (3) | 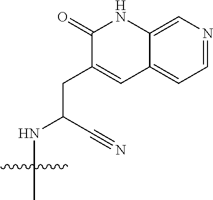 [SMILES: *NC(C#N)Cc1cc2ccncc2[nH]c1=O] (3) |
| 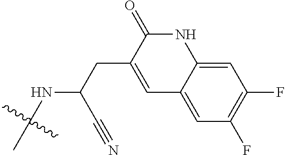 | 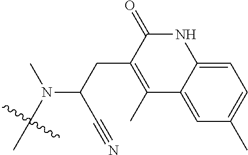 [SMILES: *N(C)C(C#N)Cc1c(C)c2cc(C)ccc2[nH]c1=O] | 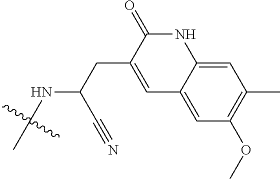 |
| (3) | (3) | (3) |
| 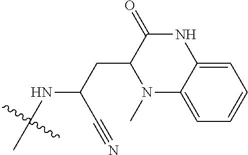 | 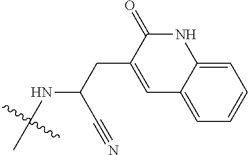 [SMILES: *NC(C#N)Cc1cc2ccccc2[nH]c1=O] | 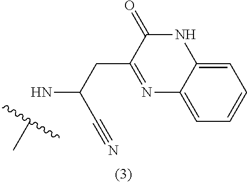 |
| (3) | (3) | |

In some embodiments, the compound is of Formula (II-I):

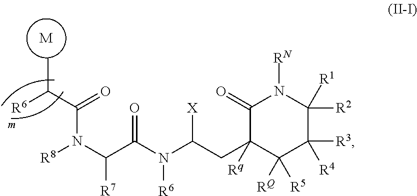

or a pharmaceutically acceptable salt, stereoisomer, solvate, prodrug, isotopic derivative, or tautomer thereof wherein all variables are as defined herein.

In some embodiments, the compound is of Formula (II-II):

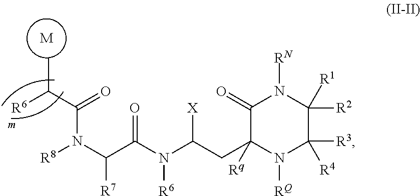

or a pharmaceutically acceptable salt, stereoisomer, solvate, prodrug, isotopic derivative, or tautomer thereof wherein all variables are as defined herein.

In some embodiments, the compound is of Formula (II-I-I):

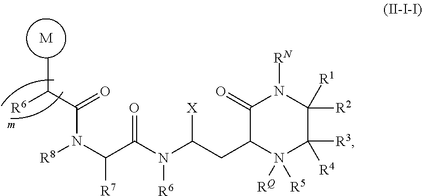

or a pharmaceutically acceptable salt, stereoisomer, solvate, prodrug, isotopic derivative, or tautomer thereof wherein all variables are as defined herein.

In some embodiments, the compound is of Formula (II-I-II):

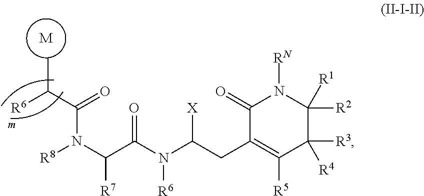

or a pharmaceutically acceptable salt, stereoisomer, solvate, prodrug, isotopic derivative, or tautomer thereof wherein all variables are as defined herein.

In some embodiments, the compound is of Formula (II-II-I):

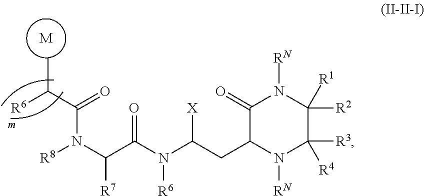

or a pharmaceutically acceptable salt, stereoisomer, solvate, prodrug, isotopic derivative, or tautomer thereof wherein all variables are as defined herein.

In some embodiments, the compound is of Formula (II-II-II):

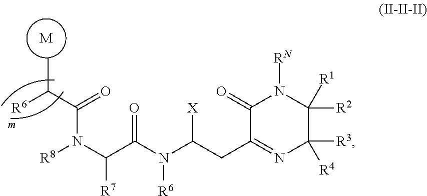

or a pharmaceutically acceptable salt, stereoisomer, solvate, prodrug, isotopic derivative, or tautomer thereof wherein all variables are as defined herein.

In some embodiments, the compound is of Formula (II-I-I-A):

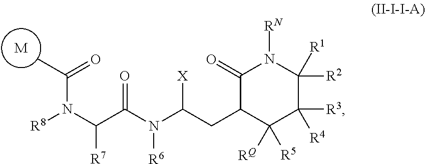

or a pharmaceutically acceptable salt, stereoisomer, solvate, prodrug, isotopic derivative, or tautomer thereof wherein all variables are as defined herein.

In some embodiments, the compound is of Formula (II-I-I-B):

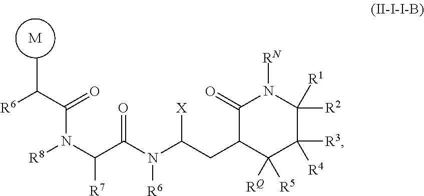

or a pharmaceutically acceptable salt, stereoisomer, solvate, prodrug, isotopic derivative, or tautomer thereof wherein all variables are as defined herein.

In some embodiments, the compound is of Formula (II-I-II-A):

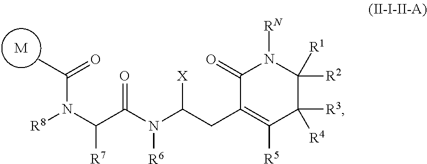

or a pharmaceutically acceptable salt, stereoisomer, solvate, prodrug, isotopic derivative, or tautomer thereof wherein all variables are as defined herein.

In some embodiments, the compound is of Formula (II-I-II-B):

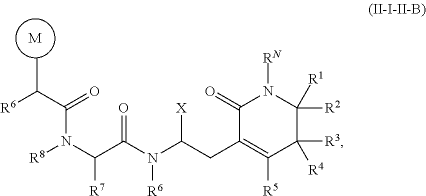

or a pharmaceutically acceptable salt, stereoisomer, solvate, prodrug, isotopic derivative, or tautomer thereof wherein all variables are as defined herein.

In some embodiments, the compound is of Formula (II-II-I-A):

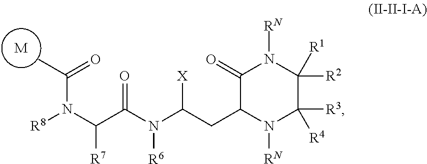

or a pharmaceutically acceptable salt, stereoisomer, solvate, prodrug, isotopic derivative, or tautomer thereof wherein all variables are as defined herein.

In some embodiments, the compound is of Formula (II-II-I-B):

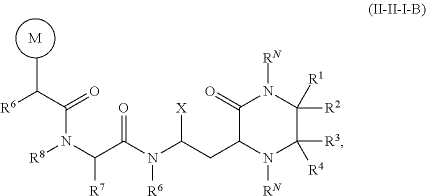

or a pharmaceutically acceptable salt, stereoisomer, solvate, prodrug, isotopic derivative, or tautomer thereof wherein all variables are as defined herein.

In some embodiments, the compound is of Formula (II-II-II-A):

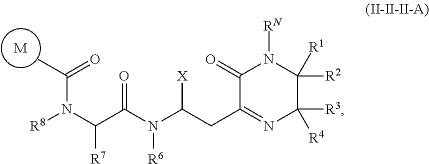

or a pharmaceutically acceptable salt, stereoisomer, solvate, prodrug, isotopic derivative, or tautomer thereof wherein all variables are as defined herein.

In some embodiments, the compound is of Formula (II-II-II-B):

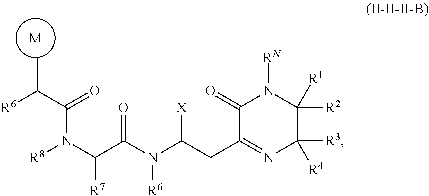

or a pharmaceutically acceptable salt, stereoisomer, solvate, prodrug, isotopic derivative, or tautomer thereof wherein all variables are as defined herein.

In some embodiments, the compound is of Formula (II-I-I-A-1):

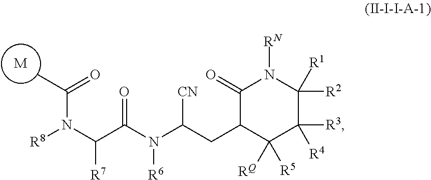

or a pharmaceutically acceptable salt, stereoisomer, solvate, prodrug, isotopic derivative, or tautomer thereof wherein all variables are as defined herein.

In some embodiments, the compound is of Formula (II-I-I-B-1):

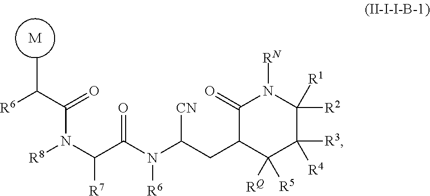

or a pharmaceutically acceptable salt, stereoisomer, solvate, prodrug, isotopic derivative, or tautomer thereof wherein all variables are as defined herein.

In some embodiments, the compound is of Formula (II-I-II-A-1):

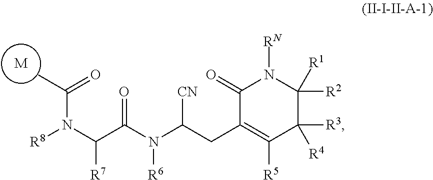

or a pharmaceutically acceptable salt, stereoisomer, solvate, prodrug, isotopic derivative, or tautomer thereof wherein all variables are as defined herein.

In some embodiments, the compound is of Formula (II-I-II-B-1):

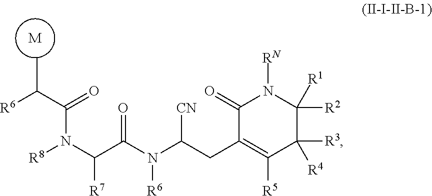

or a pharmaceutically acceptable salt, stereoisomer, solvate, prodrug, isotopic derivative, or tautomer thereof wherein all variables are as defined herein.

In some embodiments, the compound is of Formula (II-II-I-A-1):

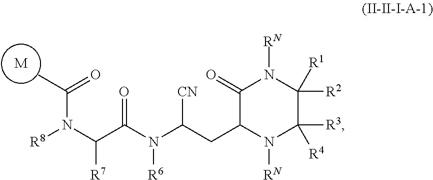

or a pharmaceutically acceptable salt, stereoisomer, solvate, prodrug, isotopic derivative, or tautomer thereof wherein all variables are as defined herein.

In some embodiments, the compound is of Formula (II-II-I-B-1):

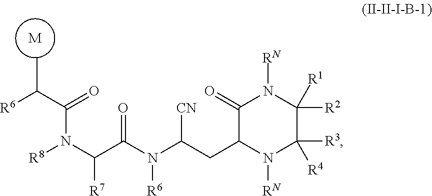

or a pharmaceutically acceptable salt, stereoisomer, solvate, prodrug, isotopic derivative, or tautomer thereof wherein all variables are as defined herein.

In some embodiments, the compound is of Formula (II-II-II-A-1):

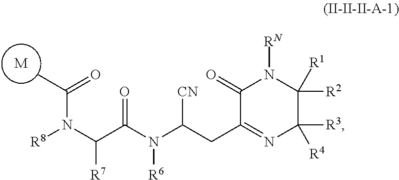

or a pharmaceutically acceptable salt, stereoisomer, solvate, prodrug, isotopic derivative, or tautomer thereof wherein all variables are as defined herein.

In some embodiments, the compound is of Formula (II-II-II-B-1):

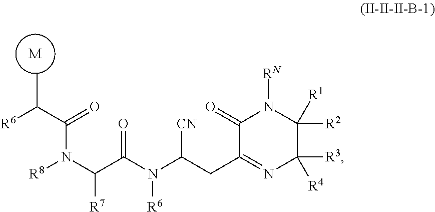

or a pharmaceutically acceptable salt, stereoisomer, solvate, prodrug, isotopic derivative, or tautomer thereof wherein all variables are as defined herein.

In some embodiments, the compound is of Formula (II-I-I-A-1-a):

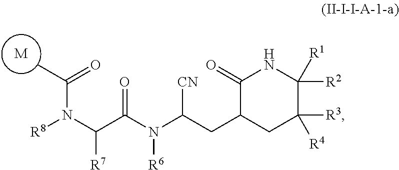

or a pharmaceutically acceptable salt, stereoisomer, solvate, prodrug, isotopic derivative, or tautomer thereof wherein all variables are as defined herein.

In some embodiments, the compound is of Formula (II-I-I-B-1-a):

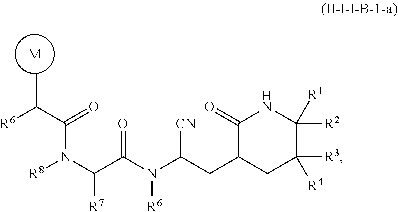

or a pharmaceutically acceptable salt, stereoisomer, solvate, prodrug, isotopic derivative, or tautomer thereof wherein all variables are as defined herein.

In some embodiments, the compound is of Formula (II-I-II-A-1-a):

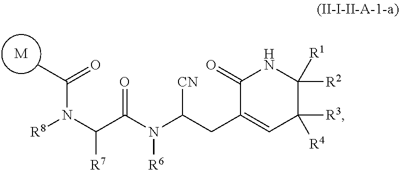

or a pharmaceutically acceptable salt, stereoisomer, solvate, prodrug, isotopic derivative, or tautomer thereof wherein all variables are as defined herein.

In some embodiments, the compound is of Formula (II-I-II-B-1-a):

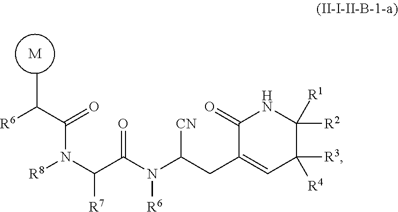

or a pharmaceutically acceptable salt, stereoisomer, solvate, prodrug, isotopic derivative, or tautomer thereof wherein all variables are as defined herein.

In some embodiments, the compound is of Formula (II-II-I-A-1-a):

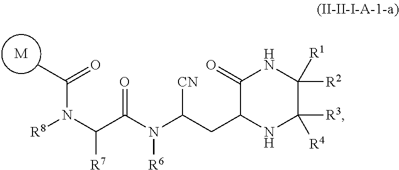

or a pharmaceutically acceptable salt, stereoisomer, solvate, prodrug, isotopic derivative, or tautomer thereof wherein all variables are as defined herein.

In some embodiments, the compound is of Formula (II-II-I-B-1-a):

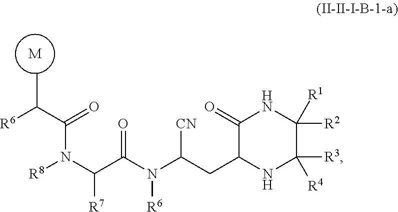

or a pharmaceutically acceptable salt, stereoisomer, solvate, prodrug, isotopic derivative, or tautomer thereof wherein all variables are as defined herein.

In some embodiments, the compound is of Formula (II-II-II-A-1-a):

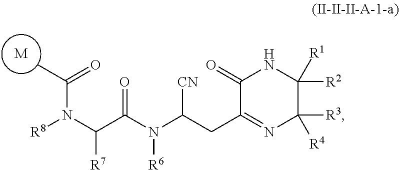

or a pharmaceutically acceptable salt, stereoisomer, solvate, prodrug, isotopic derivative, or tautomer thereof wherein all variables are as defined herein.

In some embodiments, the compound is of Formula (II-II-II-B-1-a):

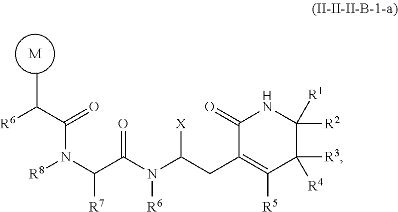

or a pharmaceutically acceptable salt, stereoisomer, solvate, prodrug, isotopic derivative, or tautomer thereof wherein all variables are as defined herein.

In some embodiments, the compound is of Formula (II-I-I-A-1-a-I):

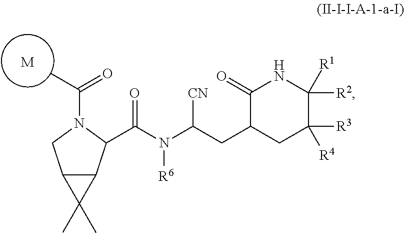

or a pharmaceutically acceptable salt, stereoisomer, solvate, prodrug, isotopic derivative, or tautomer thereof wherein all variables are as defined herein.

In some embodiments, the compound is of Formula (II-I-I-B-1-a-I):

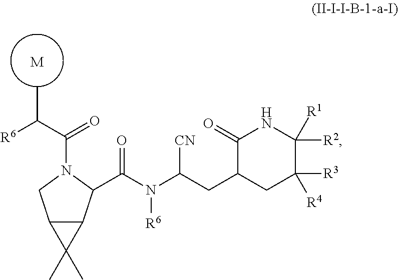

or a pharmaceutically acceptable salt, stereoisomer, solvate, prodrug, isotopic derivative, or tautomer thereof wherein all variables are as defined herein.

In some embodiments, the compound is of Formula (II-I-II-A-1-a-I):

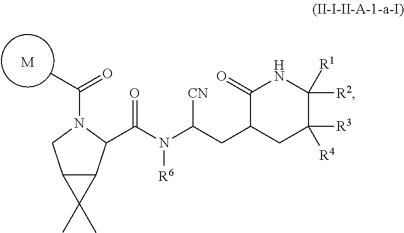

or a pharmaceutically acceptable salt, stereoisomer, solvate, prodrug, isotopic derivative, or tautomer thereof wherein all variables are as defined herein.

In some embodiments, the compound is of Formula (II-I-II-B-1-a-I):

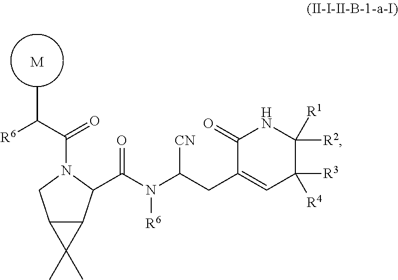

or a pharmaceutically acceptable salt, stereoisomer, solvate, prodrug, isotopic derivative, or tautomer thereof wherein all variables are as defined herein.

In some embodiments, the compound is of Formula (II-II-I-A-1-a-I):

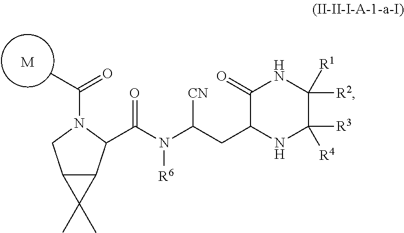

or a pharmaceutically acceptable salt, stereoisomer, solvate, prodrug, isotopic derivative, or tautomer thereof wherein all variables are as defined herein.

In some embodiments, the compound is of Formula (II-II-I-B-1-a-I):

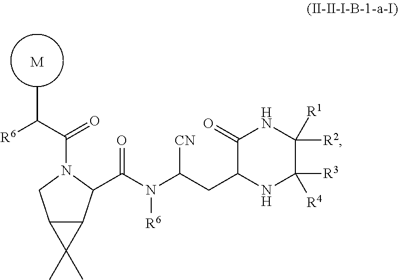

or a pharmaceutically acceptable salt, stereoisomer, solvate, prodrug, isotopic derivative, or tautomer thereof wherein all variables are as defined herein.

In some embodiments, the compound is of Formula (II-II-II-A-1-a-I):

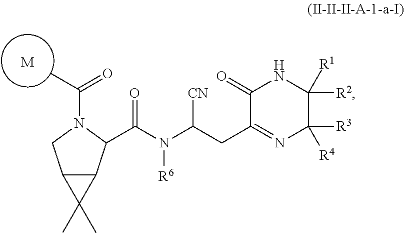

or a pharmaceutically acceptable salt, stereoisomer, solvate, prodrug, isotopic derivative, or tautomer thereof wherein all variables are as defined herein.

In some embodiments, the compound is of Formula (II-II-II-B-1-a-I):

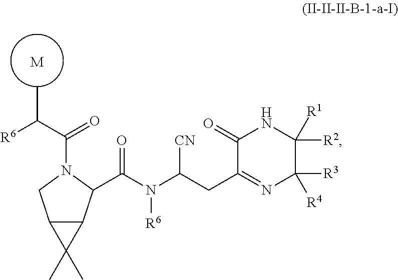

or a pharmaceutically acceptable salt, stereoisomer, solvate, prodrug, isotopic derivative, or tautomer thereof wherein all variables are as defined herein.

In some embodiments, the compound is of Formula (II-I-I-A-1-a-I-A):

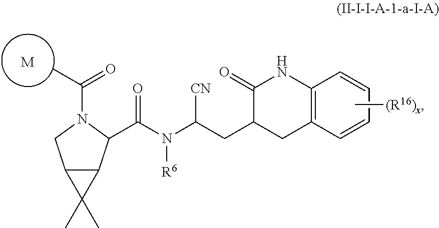

or a pharmaceutically acceptable salt, stereoisomer, solvate, prodrug, isotopic derivative, or tautomer thereof wherein all variables are as defined herein.

In some embodiments, the compound is of Formula (II-I-I-B-1-a-I-A):

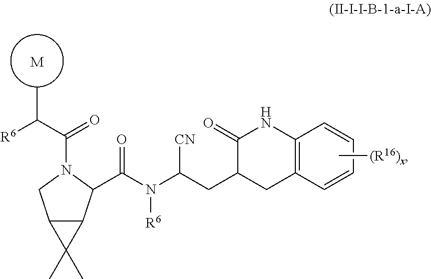

or a pharmaceutically acceptable salt, stereoisomer, solvate, prodrug, isotopic derivative, or tautomer thereof wherein all variables are as defined herein.

In some embodiments, the compound is of Formula (II-I-II-A-1-a-I-A):

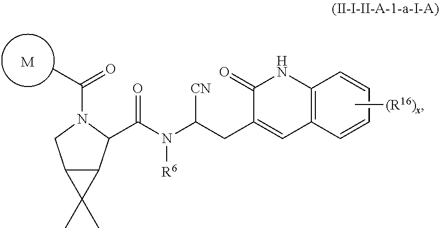

or a pharmaceutically acceptable salt, stereoisomer, solvate, prodrug, isotopic derivative, or tautomer thereof wherein all variables are as defined herein.

In some embodiments, the compound is of Formula (II-I-II-B-1-a-I-A):

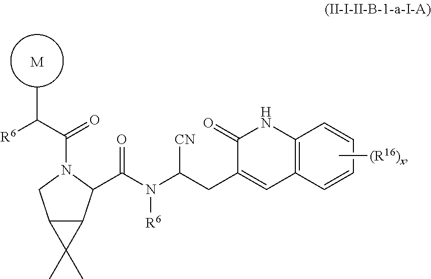

or a pharmaceutically acceptable salt, stereoisomer, solvate, prodrug, isotopic derivative, or tautomer thereof wherein all variables are as defined herein.

In some embodiments, the compound is of Formula (II-II-I-A-1-a-I-A):

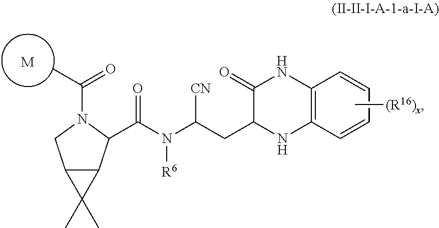

or a pharmaceutically acceptable salt, stereoisomer, solvate, prodrug, isotopic derivative, or tautomer thereof wherein all variables are as defined herein.

In some embodiments, the compound is of Formula (II-II-I-B-1-a-I-A):

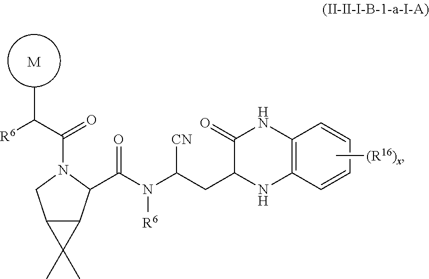

or a pharmaceutically acceptable salt, stereoisomer, solvate, prodrug, isotopic derivative, or tautomer thereof wherein all variables are as defined herein.

In some embodiments, the compound is of Formula (II-II-II-A-1-a-I-A):

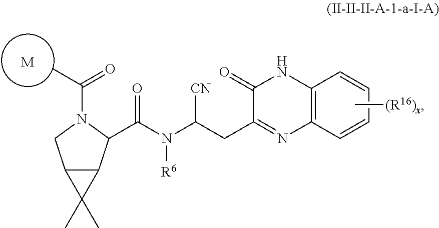

or a pharmaceutically acceptable salt, stereoisomer, solvate, prodrug, isotopic derivative, or tautomer thereof wherein all variables are as defined herein.

In some embodiments, the compound is of Formula (II-II-II-B-1-a-I-A):

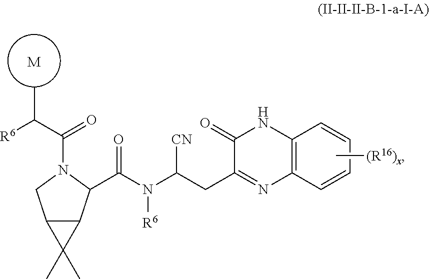

or a pharmaceutically acceptable salt, stereoisomer, solvate, prodrug, isotopic derivative, or tautomer thereof wherein all variables are as defined herein.

In some embodiments, the compound is of Formula (II-I-I-A-1-a-I-A-1):

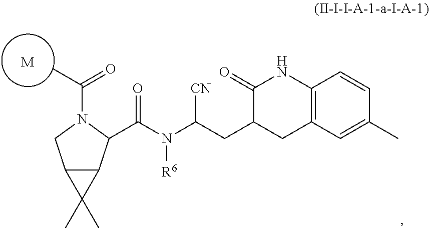

or a pharmaceutically acceptable salt, stereoisomer, solvate, prodrug, isotopic derivative, or tautomer thereof wherein all variables are as defined herein.

In some embodiments, the compound is of Formula (II-I-I-B-1-a-I-A-1):

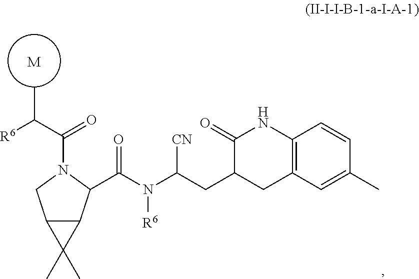

or a pharmaceutically acceptable salt, stereoisomer, solvate, prodrug, isotopic derivative, or tautomer thereof wherein all variables are as defined herein.

In some embodiments, the compound is of Formula (II-I-II-A-1-a-I-A-1):

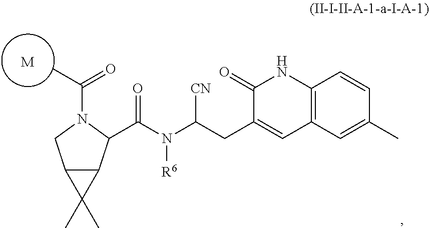

or a pharmaceutically acceptable salt, stereoisomer, solvate, prodrug, isotopic derivative, or tautomer thereof wherein all variables are as defined herein.

In some embodiments, the compound is of Formula (II-I-II-B-1-a-I-A-1):

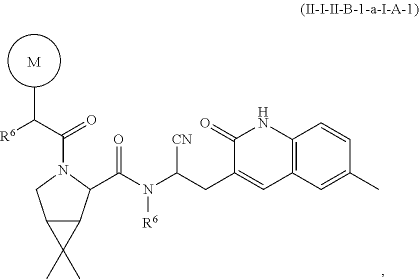

or a pharmaceutically acceptable salt, stereoisomer, solvate, prodrug, isotopic derivative, or tautomer thereof wherein all variables are as defined herein.

In some embodiments, the compound is of Formula (II-II-I-A-1-a-I-A-1):

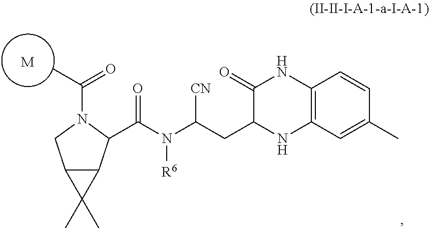

or a pharmaceutically acceptable salt, stereoisomer, solvate, prodrug, isotopic derivative, or tautomer thereof wherein all variables are as defined herein.

In some embodiments, the compound is of Formula (II-II-I-B-1-a-I-A-1):

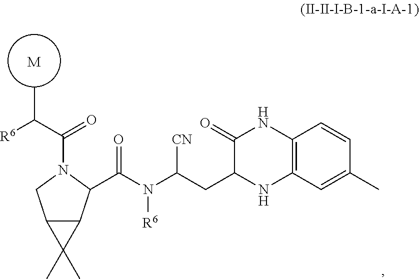

or a pharmaceutically acceptable salt, stereoisomer, solvate, prodrug, isotopic derivative, or tautomer thereof wherein all variables are as defined herein.

In some embodiments, the compound is of Formula (II-II-II-A-1-a-I-A-1):

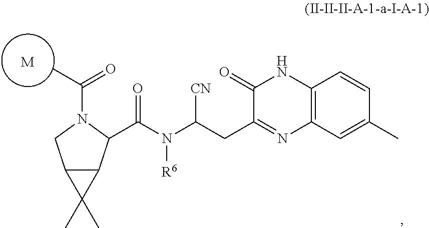

or a pharmaceutically acceptable salt, stereoisomer, solvate, prodrug, isotopic derivative, or tautomer thereof wherein all variables are as defined herein.

In some embodiments, the compound is of Formula (II-II-II-B-1-a-I-A-1):

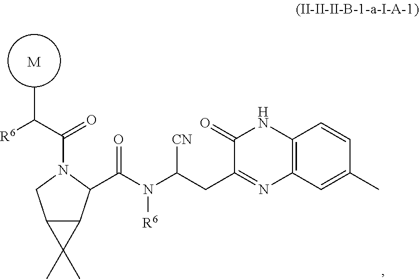

or a pharmaceutically acceptable salt, stereoisomer, solvate, prodrug, isotopic derivative, or tautomer thereof wherein all variables are as defined herein.

In some embodiments, the compound is of Formula (II-I-I-A-1-a-I-B):

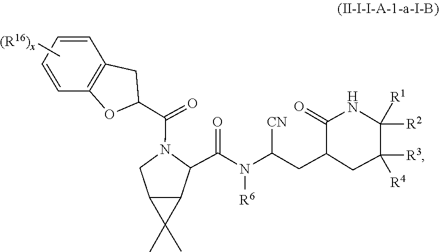

or a pharmaceutically acceptable salt, stereoisomer, solvate, prodrug, isotopic derivative, or tautomer thereof wherein x is an integer selected from 0, 1, 2, 3, and 4; and all other variables are as defined herein.

In some embodiments, the compound is of Formula (II-I-I-B-1-a-I-B):

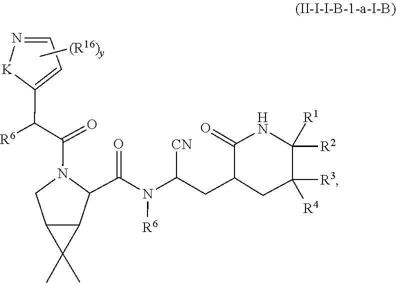

or a pharmaceutically acceptable salt, stereoisomer, solvate, prodrug, isotopic derivative, or tautomer thereof wherein K is selected from O, NH, S; y is an integer selected from 0, 1, and 2; and all other variables are as defined herein.

In some embodiments, the compound is of Formula (II-I-II-A-1-a-I-B):

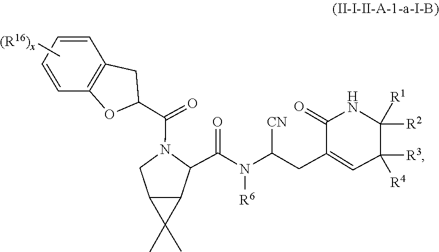

or a pharmaceutically acceptable salt, stereoisomer, solvate, prodrug, isotopic derivative, or tautomer thereof, wherein x is an integer selected from 0, 1, 2, 3, and 4; and all other variables are as defined herein.

In some embodiments, the compound is of Formula (II-I-II-B-1-a-I-B):

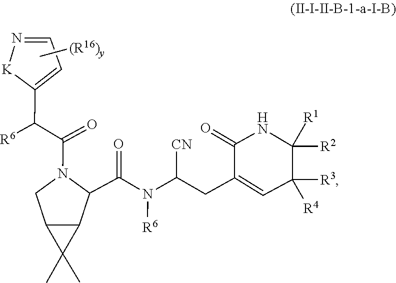

or a pharmaceutically acceptable salt, stereoisomer, solvate, prodrug, isotopic derivative, or tautomer thereof, wherein K is selected from O, NH, S; y is an integer selected from 0, 1, and 2; and all other variables are as defined herein.

In some embodiments, the compound is of Formula (II-II-I-A-1-a-I-B):

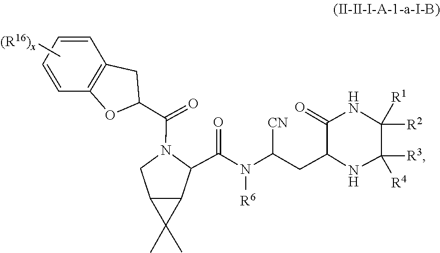

or a pharmaceutically acceptable salt, stereoisomer, solvate, prodrug, isotopic derivative, or tautomer thereof, wherein x is an integer selected from 0, 1, 2, 3, and 4; and all other variables are as defined herein.

In some embodiments, the compound is of Formula (II-II-I-B-1-a-I-B):

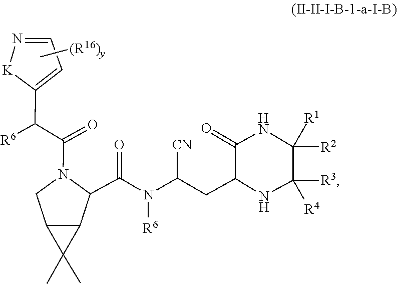

or a pharmaceutically acceptable salt, stereoisomer, solvate, prodrug, isotopic derivative, or tautomer thereof, wherein K is selected from O, NH, S; y is an integer selected from 0, 1, and 2; and all other variables are as defined herein.

In some embodiments, the compound is of Formula (II-II-II-A-1-a-I-B):

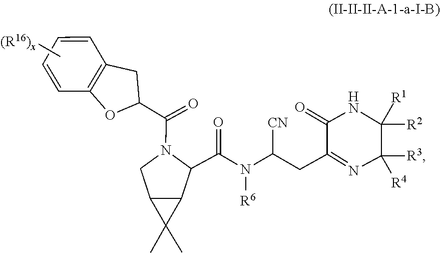

or a pharmaceutically acceptable salt, stereoisomer, solvate, prodrug, isotopic derivative, or tautomer thereof, wherein x is an integer selected from 0, 1, 2, 3, and 4; and all other variables are as defined herein.

In some embodiments, the compound is of Formula (II-II-II-B-1-a-I-B):

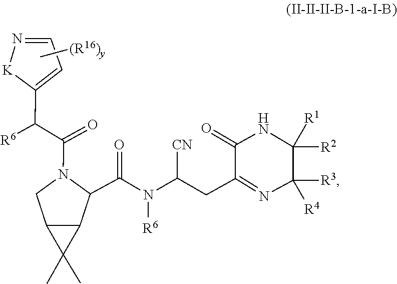

or a pharmaceutically acceptable salt, stereoisomer, solvate, prodrug, isotopic derivative, or tautomer thereof wherein all variables are as defined herein.

In some embodiments, the compound of Formula (II) is any combination of fragments (1), (2) and (3), presented in the Table 2.

TABLE 2
Lists of fragments (1), (2), and (3) that give the structure of the compound
of Formula (II) by combining any of fragment (1) with any of fragment (2) and any of
fragment (3).
(II)

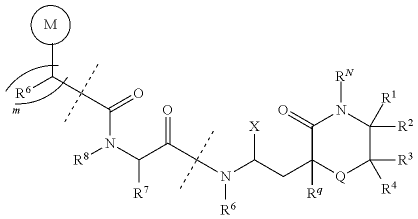

| | | |
|---|---|---|
| 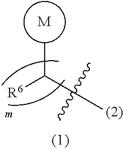 | 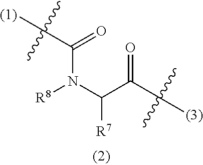 | 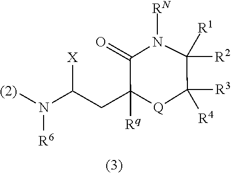 |

(1)

| | | |
|---|---|---|
| 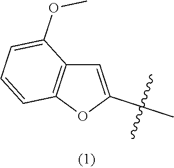 [SMILES: *c1cc2c(OC)cccc2o1] | 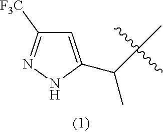 [SMILES: *C(C)c1cc(C(F)(F)F)n[nH]1] | 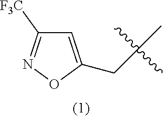 [SMILES: *Cc1cc(C(F)(F)F)no1] |
| 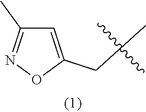 [SMILES: *Cc1cc(C)no1] | 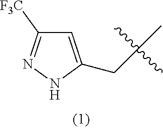 [SMILES: *Cc1cc(C(F)(F)F)n[nH]1] | 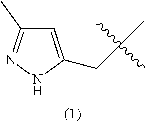 [SMILES: *Cc1cc(C)n[nH]1] |

(2)

| | | |
|---|---|---|
| 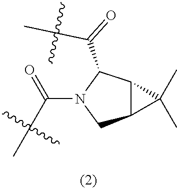 [SMILES: *C(=O)[C@@H]1[C@H]2[C@H](CN1C(*)=O)C2(C)C] | 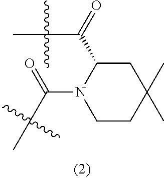 [SMILES: *C(=O)[C@@H]1CC(C)(C)CCN1C(*)=O] | 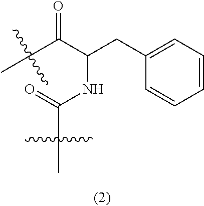 [SMILES: *C(=O)NC(Cc1ccccc1)C(*)=O] |
| 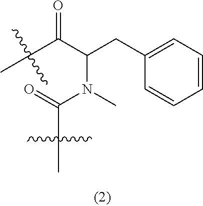 [SMILES: *C(=O)C(Cc1ccccc1)N(C)C(*)=O] | 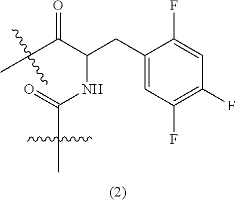 [SMILES: *C(=O)NC(Cc1cc(F)c(F)cc1F)C(*)=O] | 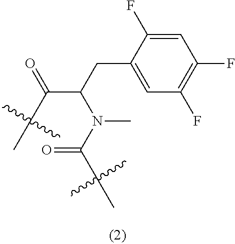 [SMILES: *C(=O)C(Cc1cc(F)c(F)cc1F)N(C)C(*)=O] |

| | |
|---|---|
| 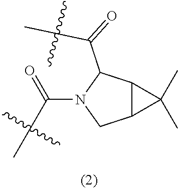 [SMILES: *C(=O)C1C2C(CN1C(*)=O)C2(C)C] | 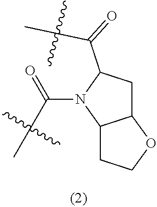 [SMILES: *C(=O)C1CC2OCCC2N1C(*)=O] |

(3)

| | | |
|---|---|---|
| 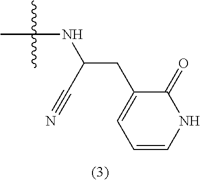 [SMILES: *NC(C#N)Cc1ccc[nH]c1=O] | 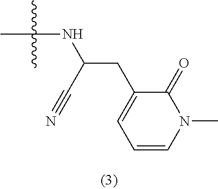 [SMILES: *NC(C#N)Cc1cccn(C)c1=O] | 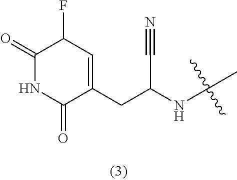 [SMILES: *NC(C#N)CC1=CC(F)C(=O)NC1=O] |
| 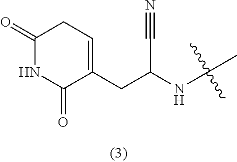 [SMILES: *NC(C#N)CC1=CCC(=O)NC1=O] | 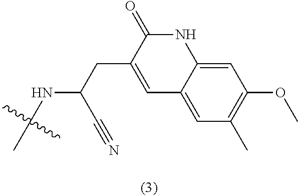 | 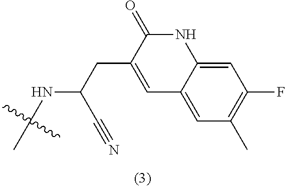 |
| 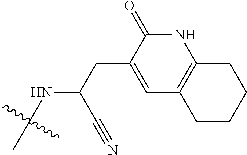 [SMILES: *NC(C#N)Cc1cc2c([nH]c1=O)CCCC2] | 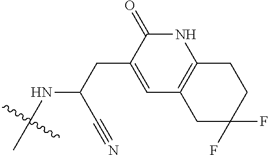 [SMILES: *NC(C#N)Cc1cc2c([nH]c1=O)CCC(F)(F)C2] | 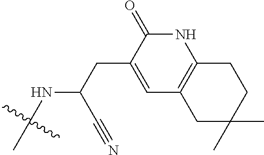 [SMILES: *NC(C#N)Cc1cc2c([nH]c1=O)CCC(C)(C)C2] |
| (3) | (3) | (3) |
| 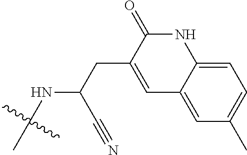 [SMILES: *NC(C#N)Cc1cc2cc(C)ccc2[nH]c1=O] | 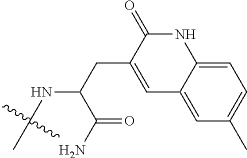 [SMILES: *NC(Cc1cc2cc(C)ccc2[nH]c1=O)C(N)=O] | 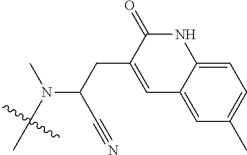 [SMILES: *N(C)C(C#N)Cc1cc2cc(C)ccc2[nH]c1=O] |
| (3) | (3) | (3) |
| 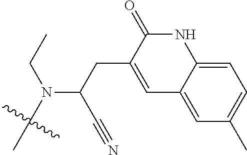 [SMILES: *N(CC)C(C#N)Cc1cc2cc(C)ccc2[nH]c1=O] | 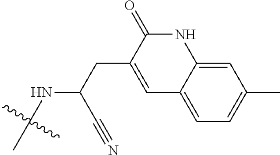 [SMILES: *NC(C#N)Cc1cc2ccc(C)cc2[nH]c1=O] | 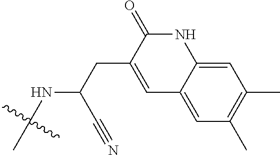 [SMILES: *NC(C#N)Cc1cc2cc(C)c(C)cc2[nH]c1=O] |
| (3) | (3) | (3) |
| 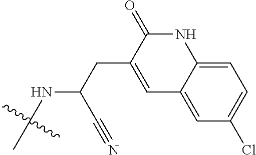 [SMILES: *NC(C#N)Cc1cc2cc(Cl)ccc2[nH]c1=O] (3) | 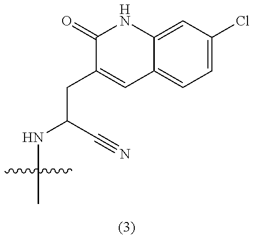 [SMILES: *NC(C#N)Cc1cc2ccc(Cl)cc2[nH]c1=O] | 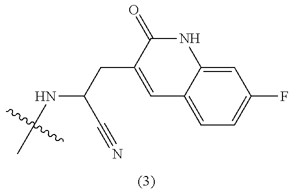 |
| 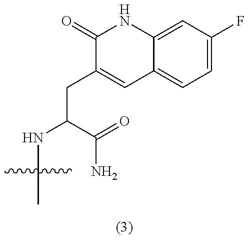 [SMILES: *NC(Cc1cc2ccc(F)cc2[nH]c1=O)C(N)=O] | 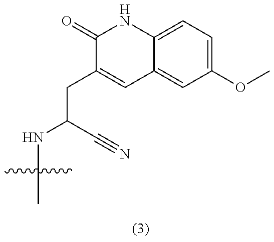 [SMILES: *NC(C#N)Cc1cc2cc(OC)ccc2[nH]c1=O] | 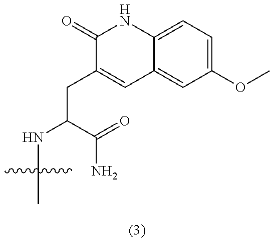 [SMILES: *NC(Cc1cc2cc(OC)ccc2[nH]c1=O)C(N)=O] |
| 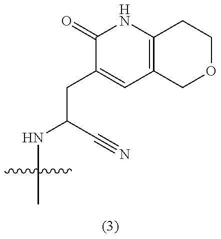 [SMILES: *NC(C#N)Cc1cc2c([nH]c1=O)CCOC2] | 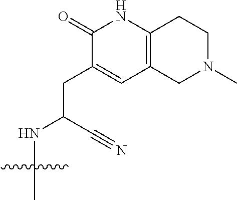 [SMILES: *NC(C#N)Cc1cc2c([nH]c1=O)CCN(C)C2] (3) | 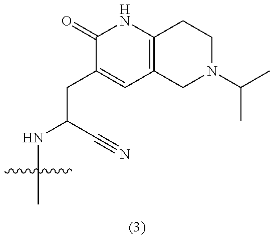 [SMILES: *NC(C#N)Cc1cc2c([nH]c1=O)CCN(C(C)C)C2] |
| 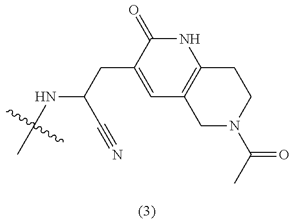 | 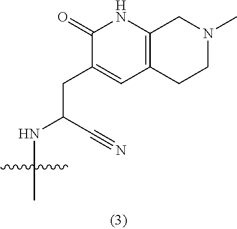 [SMILES: *NC(C#N)Cc1cc2c([nH]c1=O)CN(C)CC2] | 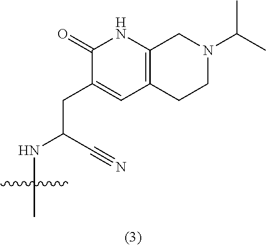 [SMILES: *NC(C#N)Cc1cc2c([nH]c1=O)CN(C(C)C)CC2] |
| 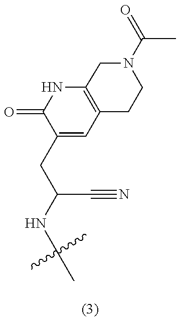 [SMILES: *NC(C#N)Cc1cc2c([nH]c1=O)CN(C(C)=O)CC2] | 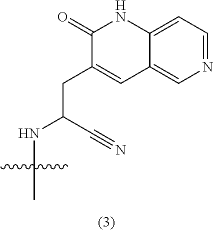 [SMILES: *NC(C#N)Cc1cc2cnccc2[nH]c1=O] | 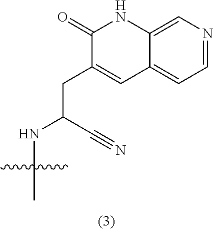 [SMILES: *NC(C#N)Cc1cc2ccncc2[nH]c1=O] |
| 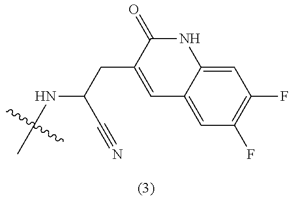 | 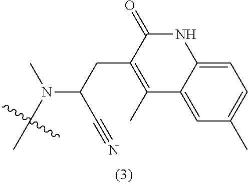 [SMILES: *N(C)C(C#N)Cc1c(C)c2cc(C)ccc2[nH]c1=O] | 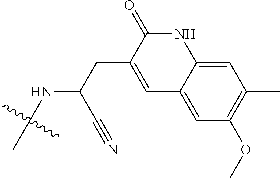 |
| | | (3) |
| 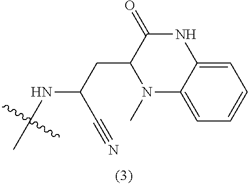 | 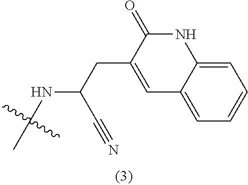 [SMILES: *NC(C#N)Cc1cc2ccccc2[nH]c1=O] | 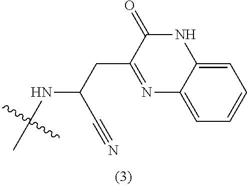 |

In some embodiments, the compound is of Formula (A′):

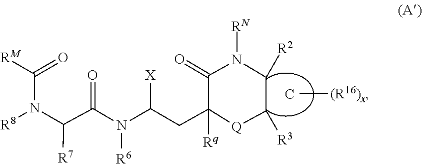

or a pharmaceutically acceptable salt, stereoisomer, solvate, prodrug, or tautomer thereof, wherein:
   each R^{N} is independently selected from H, C₁-C₆ alkyl, cycloalkyl;
   Q is CR⁵R^{Q} or NR^{Q};
   R^{q} is H;
   R^{Q} is R^{N}; or
   R^{q} and R^{Q} together form a bond;
   R^{M} is selected from

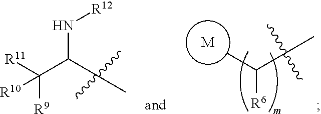

M is selected from 5-10 membered heterocyclyl or 5-10 membered heteroaryl, wherein the heterocyclyl or heteroaryl is optionally substituted with one or more R¹⁶;
C is a ring selected from cycloalkyl, aryl, heterocyclyl or heteroaryl;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R² and R³ together form a double bond;
R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O— cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
each R⁶ is independently selected from H, C₁-C₆ alkyl, cycloalkyl, —CH₂-aryl, wherein the alkyl, cycloalkyl, or aryl is optionally substituted with one or more R¹⁶;
R⁷ is selected from —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
or R⁷ and R⁸ together with the atoms to which they are attached and any intervening atoms, form 5-7 membered monocyclic or bicyclic heterocyclyl optionally substituted with one or more R¹³;
R⁹, R¹⁰, R¹¹ are each independently selected from C₁-C₆ alkyl, cycloalkyl, halogen, —CN;
R¹² is selected from R⁶—S(O)₂—, R⁶—C(O)—, R⁶—O—C(O)—, heteroaryl, wherein the heteroaryl is optionally substituted with one or more R¹⁶;
each R¹³ is independently selected from halogen, —OH, —CN, —C₁-C₆ alkyl, —C₁-C₆ halogenalkyl;
two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
R¹⁴ and R¹⁵ are each independently selected from H, C₁-C₆ alkyl, cycloalkyl;
or R¹⁴ and R¹⁵ together with the atoms to which they are attached and any intervening atoms, form heterocyclyl optionally substituted with one or more halogen, —OH, —CN, C₁-C₆ alkyl;
each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
n is an integer selected from 0, 1, 2;
m is an integer selected from 0 and 1;
x is an integer selected from 0, 1, 2, 3, and 4.

In some embodiments, the compound is of Formula (I′):

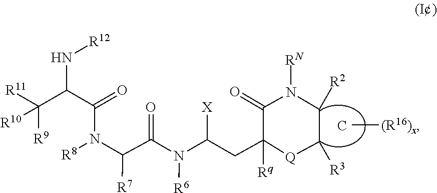

or a pharmaceutically acceptable salt, stereoisomer, solvate, prodrug, or tautomer thereof, wherein:
   each R^{N} is independently selected from H, C₁-C₆ alkyl, cycloalkyl;
   Q is CR⁵R^{Q} or NR^{Q};
   R^{q} is H;
   R^{Q} is R^{N}; or
   R^{q} and R^{Q} together form a bond;
   C is a ring selected from cycloalkyl, aryl, heterocyclyl or heteroaryl;
   R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   or R² and R³ together form a double bond;
   R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O— cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   each R⁶ is independently selected from H, C₁-C₆ alkyl, cycloalkyl, —CH₂-aryl, wherein the alkyl, cycloalkyl, or aryl is optionally substituted with one or more R¹⁶;
   R⁷ is selected from —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
   R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
   or R⁷ and R⁸ together with the atoms to which they are attached and any intervening atoms, form 5-7 membered monocyclic or bicyclic heterocyclyl optionally substituted with one or more R³;
   R⁹, R¹⁰, R¹¹ are each independently selected from C₁-C₆ alkyl, cycloalkyl, halogen, —CN;
   R¹² is selected from R⁶—S(O)₂—, R⁶—C(O)—, R⁶—O—C(O)—, heteroaryl, wherein the heteroaryl is optionally substituted with one or more R¹⁶;
   each R¹³ is independently selected from halogen, —OH, —CN, —C₁-C₆ alkyl, —C₁-C₆ halogenalkyl;
   two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
   R¹⁴ and R¹⁵ are each independently selected from H, C₁-C₆ alkyl, cycloalkyl;
   or R¹⁴ and R¹⁵ together with the atoms to which they are attached and any intervening atoms, form heterocyclyl optionally substituted with one or more halogen, —OH, —CN, C₁-C₆ alkyl;
   each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
   two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
   X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
   n is an integer selected from 0, 1, 2;
   x is an integer selected from 0, 1, 2, 3 and 4.

In some embodiments, the compound is of Formula (II′):

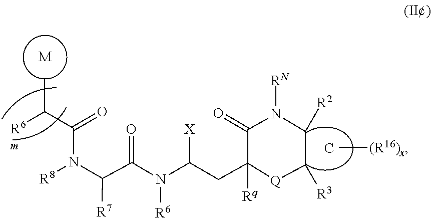

or a pharmaceutically acceptable salt, stereoisomer, solvate, prodrug, or tautomer thereof, wherein:
   each R^{N} is independently selected from H, C₁-C₆ alkyl, cycloalkyl;
   Q is CR⁵R^{Q} or NR^{Q};
   R^{q} is H;
   R^{Q} is R^{N}; or
   R^{q} and R^{Q} together form a bond;
   M is selected from 5-10 membered heterocyclyl or 5-10 membered heteroaryl, wherein the heterocyclyl or heteroaryl is optionally substituted with one or more R¹⁶;
   C is a ring selected from cycloalkyl, aryl, heterocyclyl or heteroaryl;
   R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   or R² and R³ together form a double bond;
   R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O— cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
   each R⁶ is independently selected from H, C₁-C₆ alkyl, cycloalkyl, —CH₂-aryl, wherein the alkyl, cycloalkyl, or aryl is optionally substituted with one or more R¹⁶;
   R⁷ is selected from —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
   R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
   or R⁷ and R⁸ together with the atoms to which they are attached and any intervening atoms, form 5-7 membered monocyclic or bicyclic heterocyclyl optionally substituted with one or more R³;
   each R¹³ is independently selected from halogen, —OH, —CN, —C₁-C₆ alkyl, —C₁-C₆ halogenalkyl;
   two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
   R¹⁴ and R¹⁵ are each independently selected from H, C₁-C₆ alkyl, cycloalkyl;
   or R¹⁴ and R¹⁵ together with the atoms to which they are attached and any intervening atoms, form heterocyclyl optionally substituted with one or more halogen, —OH, —CN, C₁-C₆ alkyl;
   each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
   two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
   X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
   m is an integer selected from 0 and 1;
   n is an integer selected from 0, 1, 2;
   x is an integer selected from 0, 1, 2, 3, and 4.

In some embodiments, the compound is of Formula (A′-I):

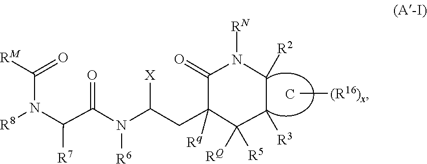

or a pharmaceutically acceptable salt, stereoisomer, solvate, prodrug, or tautomer thereof.

In some embodiments, the compound is of Formula (A′-I-a):

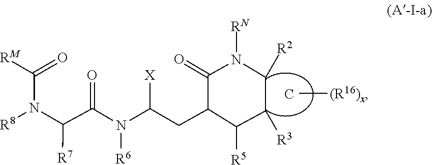

or a pharmaceutically acceptable salt, stereoisomer, solvate, prodrug, or tautomer thereof.

In some embodiments, the compound is of Formula (A′-I-b):

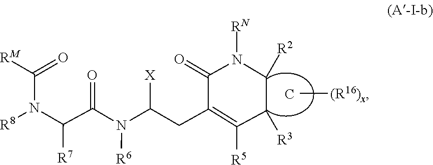

or a pharmaceutically acceptable salt, stereoisomer, solvate, prodrug, or tautomer thereof.

In some embodiments, the compound is of Formula (A′-I-a-1):

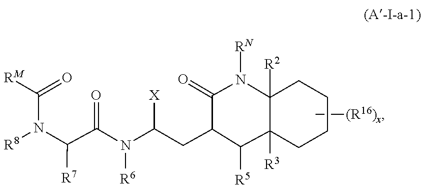

or a pharmaceutically acceptable salt, stereoisomer, solvate, prodrug, or tautomer thereof.

In some embodiments, the compound is of Formula (A′-I-a-1-A):

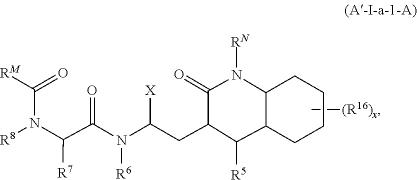

or a pharmaceutically acceptable salt, stereoisomer, solvate, prodrug, or tautomer thereof.

In some embodiments, the compound is of Formula (A′-I-a-1-B):

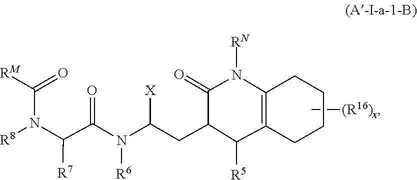

or a pharmaceutically acceptable salt, stereoisomer, solvate, prodrug, or tautomer thereof.

In some embodiments, the compound is of Formula (A′-I-a-2):

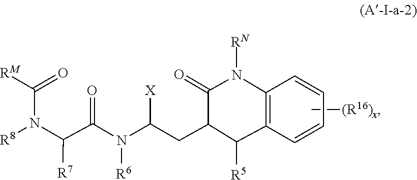

or a pharmaceutically acceptable salt, stereoisomer, solvate, prodrug, or tautomer thereof.

In some embodiments, the compound is of Formula (A′-I-a-3):

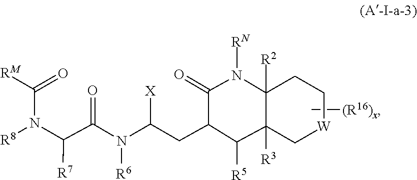

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-a-3-A):

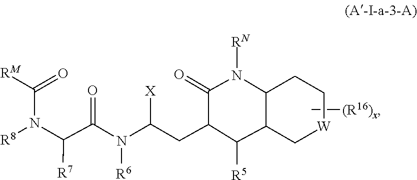

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-a-3-B):

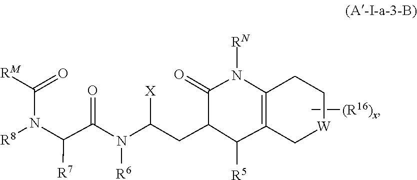

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-a-4):

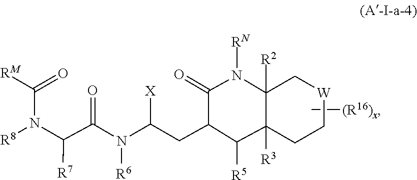

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-a-4-A):

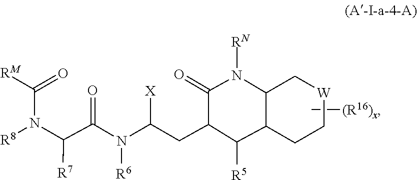

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-a-4-B):

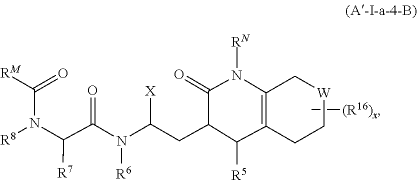

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-a-5):

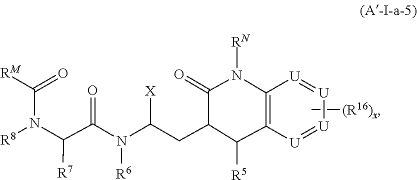

or a pharmaceutically acceptable salt, stereoisomer, solvate, prodrug, or tautomer thereof, wherein each U is independently selected from CH and N and at least one U is N; x is an integer selected from 0−(4−number of N); and all other variables are as defined herein.

In some embodiments, the compound is of Formula (A′-I-a-5-A):

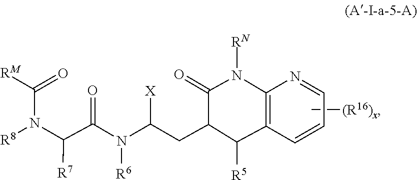

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-I-a-5-B):

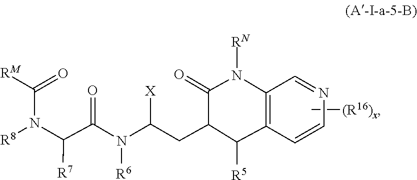

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-I-a-5-C):

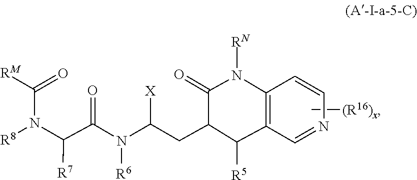

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-I-a-5-D):

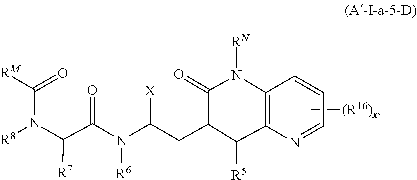

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-I-b-1):

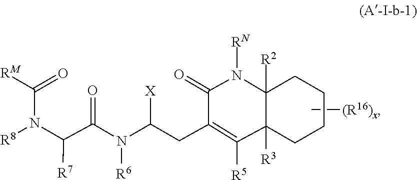

or a pharmaceutically acceptable salt, stereoisomer, solvate, prodrug, or tautomer thereof.

In some embodiments, the compound is of Formula (A′-I-b-1-A):

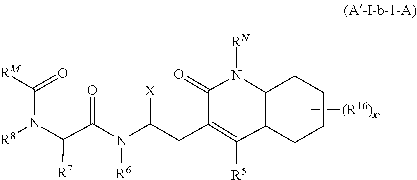

or a pharmaceutically acceptable salt, stereoisomer, solvate, prodrug, or tautomer thereof.

In some embodiments, the compound is of Formula (A′-I-b-1-B):

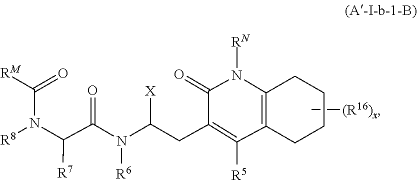

or a pharmaceutically acceptable salt, stereoisomer, solvate, prodrug, or tautomer thereof.

In some embodiments, the compound is of Formula (A′-I-b-2):

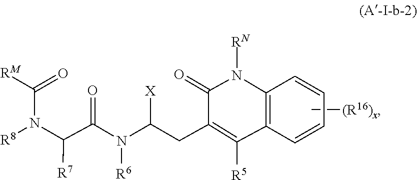

or a pharmaceutically acceptable salt, stereoisomer, solvate, prodrug, or tautomer thereof.

In some embodiments, the compound is of Formula (A′-I-b-3):

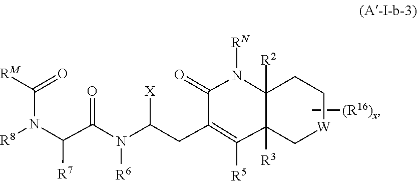

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-b-3-A):

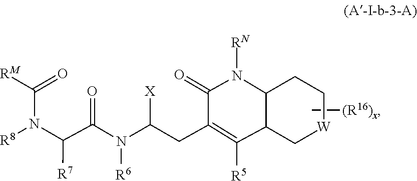

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-b-3-B):

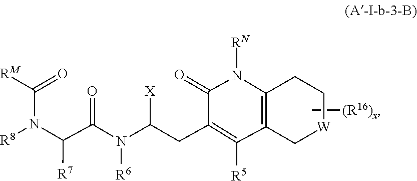

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-b-4):

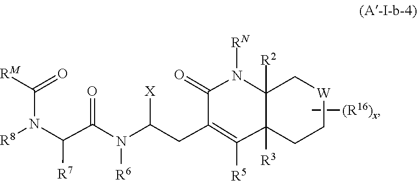

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-b-4-A):

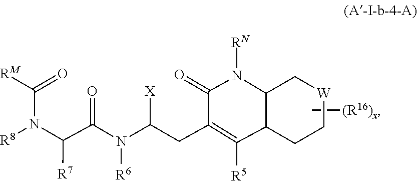

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-b-4-B):

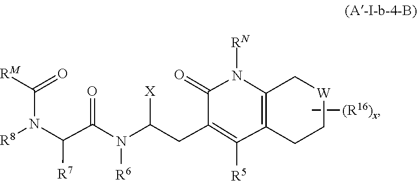

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-I-b-5):

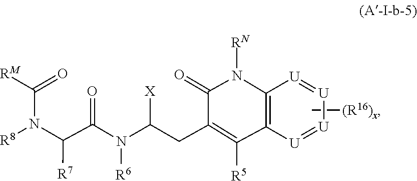

or a pharmaceutically acceptable salt, stereoisomer, solvate, prodrug, or tautomer thereof, wherein each U is independently selected from CH and N and at least one U is N; x is an integer selected from 0−(4−number of N); and all other variables are as defined herein.

In some embodiments, the compound is of Formula (A′-I-b-5-A):

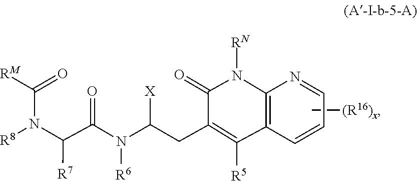

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-I-b-5-B):

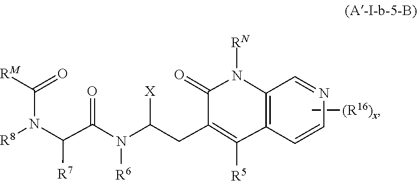

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-I-b-5-C):

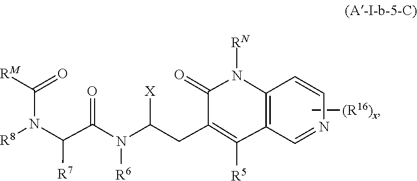

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-I-b-5-D):

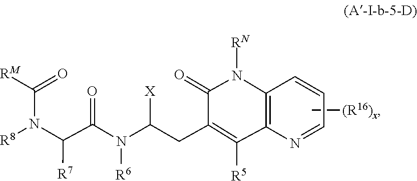

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-II):

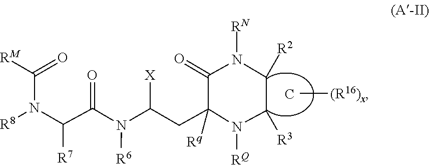

or a pharmaceutically acceptable salt, stereoisomer, solvate, prodrug, or tautomer thereof.

In some embodiments, the compound is of Formula (A′-II-a):

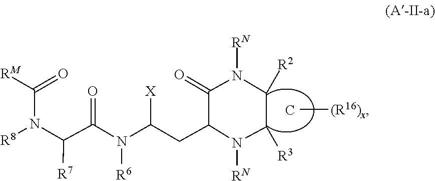

or a pharmaceutically acceptable salt, stereoisomer, solvate, prodrug, or tautomer thereof.

In some embodiments, the compound is of Formula (A′-II-b):

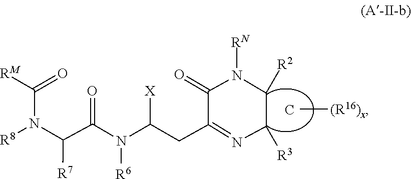

or a pharmaceutically acceptable salt, stereoisomer, solvate, prodrug, or tautomer thereof.

In some embodiments, the compound is of Formula (A′-II-a-1):

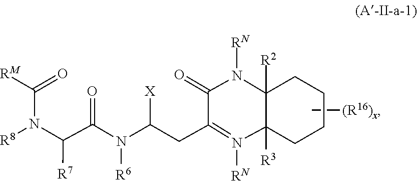

or a pharmaceutically acceptable salt, stereoisomer, solvate, prodrug, or tautomer thereof.

In some embodiments, the compound is of Formula (A′-II-a-1-A):

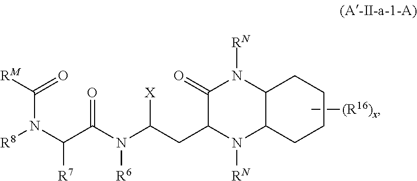

or a pharmaceutically acceptable salt, stereoisomer, solvate, prodrug, or tautomer thereof.

In some embodiments, the compound is of Formula (A′-II-a-1-B):

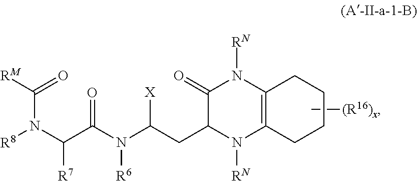

or a pharmaceutically acceptable salt, stereoisomer, solvate, prodrug, or tautomer thereof.

In some embodiments, the compound is of Formula (A′-II-a-2):

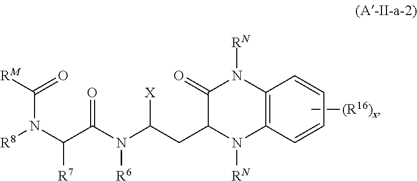

or a pharmaceutically acceptable salt, stereoisomer, solvate, prodrug, or tautomer thereof.

In some embodiments, the compound is of Formula (A′-II-a-3):

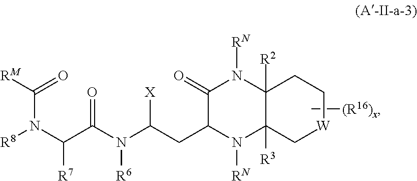

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-a-3-A):

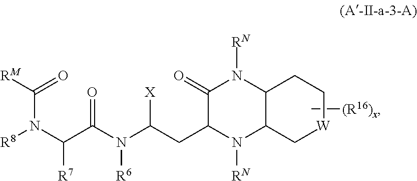

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-a-3-B):

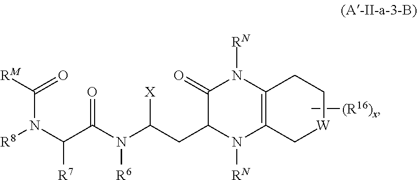

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-a-4):

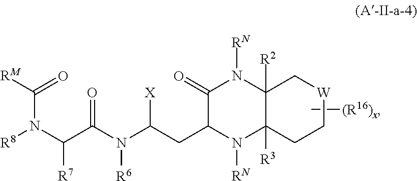

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-a-4-A):

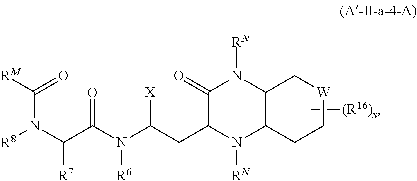

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-a-4-B):

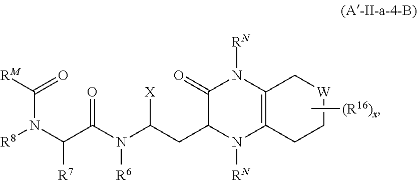

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-a-5):

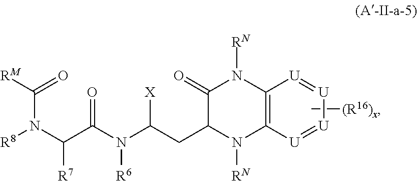

or a pharmaceutically acceptable salt, stereoisomer, solvate, prodrug, or tautomer thereof, wherein each U is independently selected from CH and N and at least one U is N; x is an integer selected from 0−(4−number of N); and all other variables are as defined herein.

In some embodiments, the compound is of Formula (A′-II-a-5-A):

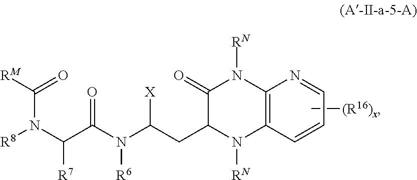

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-II-a-5-B):

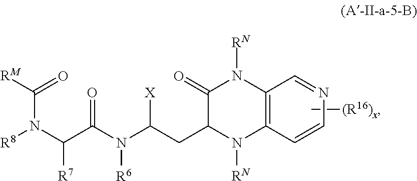

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-II-a-5-C):

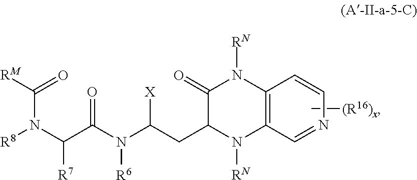

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-II-a-5-D):

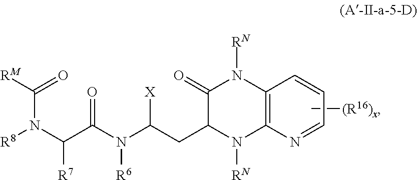

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-II-b-1):

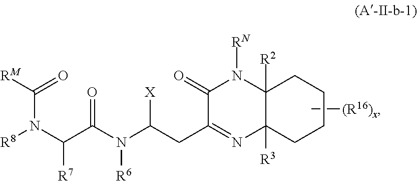

or a pharmaceutically acceptable salt, stereoisomer, solvate, prodrug, or tautomer thereof.

In some embodiments, the compound is of Formula (A′-II-b-1-A):

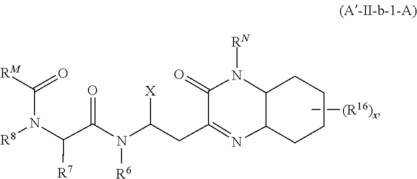

or a pharmaceutically acceptable salt, stereoisomer, solvate, prodrug, or tautomer thereof.

In some embodiments, the compound is of Formula (A′-II-b-1-B):

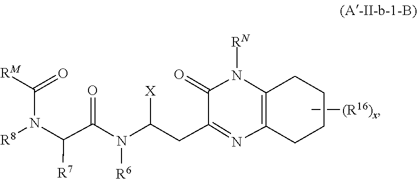

or a pharmaceutically acceptable salt, stereoisomer, solvate, prodrug, or tautomer thereof.

In some embodiments, the compound is of Formula (A′-II-b-2):

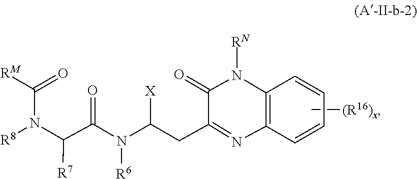

or a pharmaceutically acceptable salt, stereoisomer, solvate, prodrug, or tautomer thereof.

In some embodiments, the compound is of Formula (A′-II-b-3):

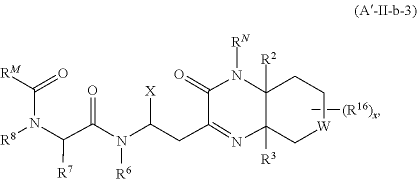

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-b-3-A):

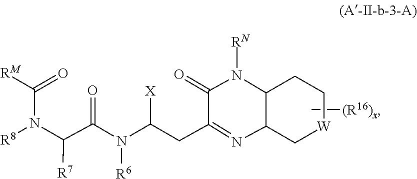

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-b-3-B):

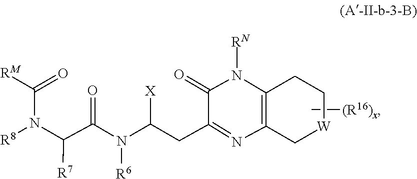

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-b-4):

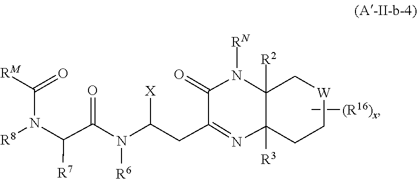

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-b-4-A):

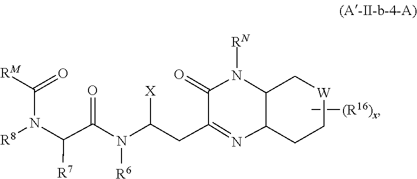

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-b-4-B):

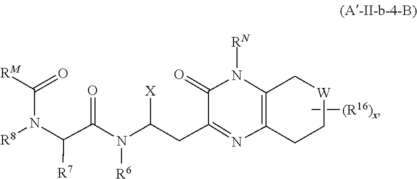

or a pharmaceutically acceptable salt, stereoisomer, solvate, prodrug, or tautomer thereof, wherein W is selected from O, NH, S, S(O), S(O)₂ and all other variables are as defined herein.

In some embodiments, the compound is of Formula (A′-II-b-5):

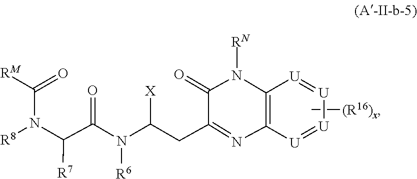

or a pharmaceutically acceptable salt, stereoisomer, solvate, prodrug, or tautomer thereof, wherein each U is independently selected from CH and N and at least one U is N; x is an integer selected from 0−(4−number of N); and all other variables are as defined herein.

In some embodiments, the compound is of Formula (A′-II-b-5-A):

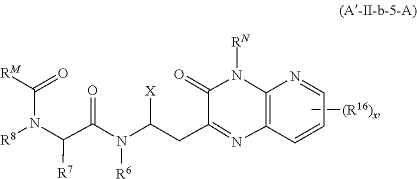

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-II-b-5-B):

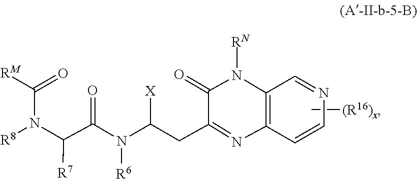

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-II-b-5-C):

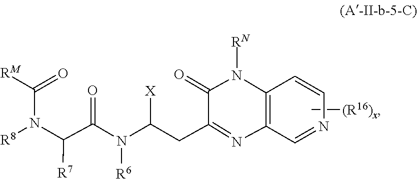

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (A′-II-b-5-D):

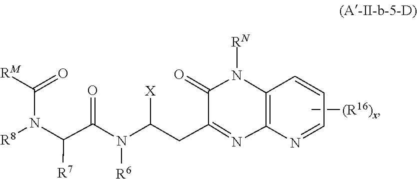

or a pharmaceutically acceptable salt, stereoisomer, solvate, prodrug, or tautomer thereof, wherein x is an integer selected from 0, 1, 2 and 3; and all other variables are as defined herein.

In some embodiments, the compound is of Formula (I′-I):

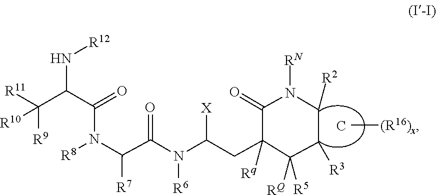

or a pharmaceutically acceptable salt, stereoisomer, solvate, prodrug, or tautomer thereof.

In some embodiments, the compound is of Formula (I′-II):

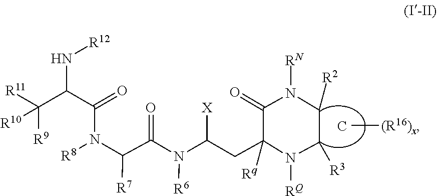

or a pharmaceutically acceptable salt, stereoisomer, solvate, prodrug, or tautomer thereof.

In some embodiments, the compound is of Formula (I′-I-a):

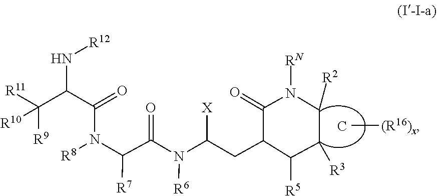

or a pharmaceutically acceptable salt, stereoisomer, solvate, prodrug, or tautomer thereof.

In some embodiments, the compound is of Formula (I′-I-b):

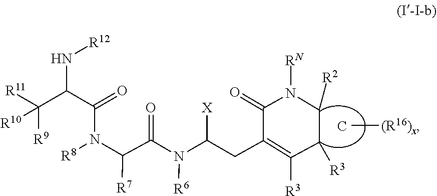

or a pharmaceutically acceptable salt, stereoisomer, solvate, prodrug, or tautomer thereof.

In some embodiments, the compound is of Formula (I′-II-a):

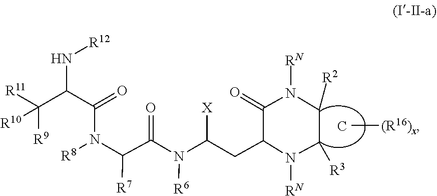

or a pharmaceutically acceptable salt, stereoisomer, solvate, prodrug, or tautomer thereof.

In some embodiments, the compound is of Formula (I′-II-b):

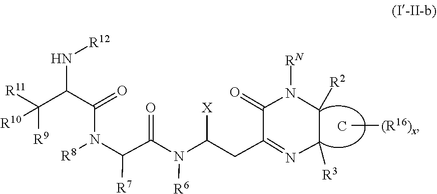

or a pharmaceutically acceptable salt, stereoisomer, solvate, prodrug, or tautomer thereof.

In some embodiments, the compound is of Formula (II′-I):

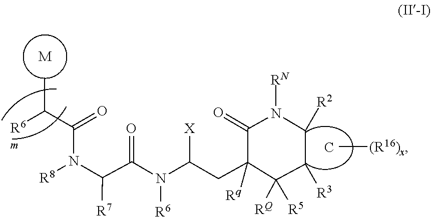

or a pharmaceutically acceptable salt, stereoisomer, solvate, prodrug, or tautomer thereof.

In some embodiments, the compound is of Formula (II′-II):

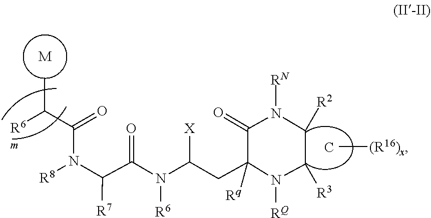

or a pharmaceutically acceptable salt, stereoisomer, solvate, prodrug, or tautomer thereof.

In some embodiments, the compound is of Formula (II′-I-a):

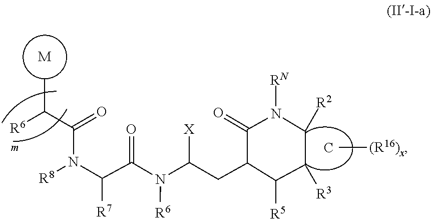

or a pharmaceutically acceptable salt, stereoisomer, solvate, prodrug, or tautomer thereof.

In some embodiments, the compound is of Formula (II′-I-b):

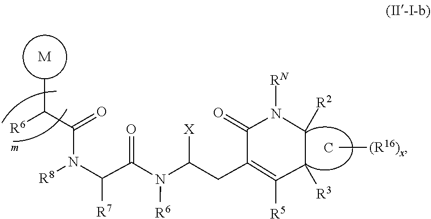

or a pharmaceutically acceptable salt, stereoisomer, solvate, prodrug, or tautomer thereof.

In some embodiments, the compound is of Formula (II′-II-a):

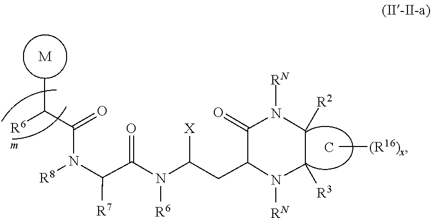

or a pharmaceutically acceptable salt, stereoisomer, solvate, prodrug, or tautomer thereof.

In some embodiments, the compound is of Formula (II′-II-b):

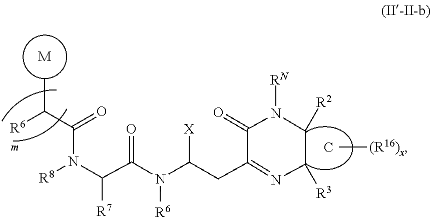

or a pharmaceutically acceptable salt, stereoisomer, solvate, prodrug, or tautomer thereof.

In some embodiments, the compound is of Formula (II′-I-a-1):

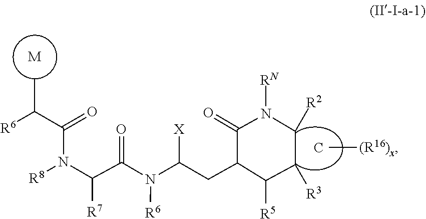

or a pharmaceutically acceptable salt, stereoisomer, solvate, prodrug, or tautomer thereof.

In some embodiments, the compound is of Formula (II′-I-a-2):

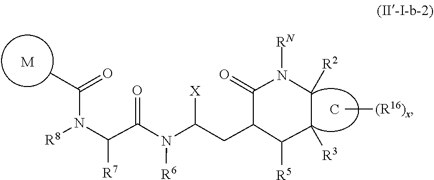

or a pharmaceutically acceptable salt, stereoisomer, solvate, prodrug, or tautomer thereof.

In some embodiments, the compound is of Formula (II′-I-b-1):

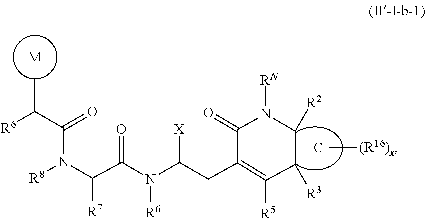

or a pharmaceutically acceptable salt, stereoisomer, solvate, prodrug, or tautomer thereof.

In some embodiments, the compound is of Formula (II′-I-b-2):

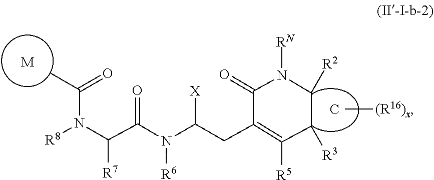

or a pharmaceutically acceptable salt, stereoisomer, solvate, prodrug, or tautomer thereof.

In some embodiments, the compound is of Formula (II′-II-a-1):

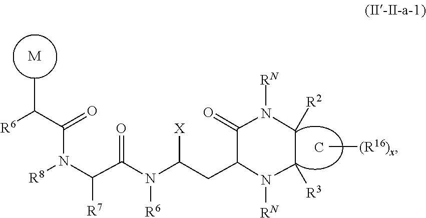

or a pharmaceutically acceptable salt, stereoisomer, solvate, prodrug, or tautomer thereof.

In some embodiments, the compound is of Formula (II′-II-a-2):

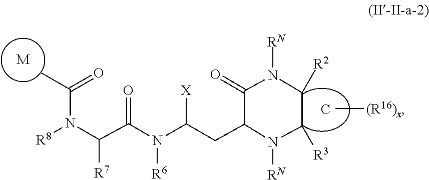

or a pharmaceutically acceptable salt, stereoisomer, solvate, prodrug, or tautomer thereof.

In some embodiments, the compound is of Formula (II′-II-b-1):

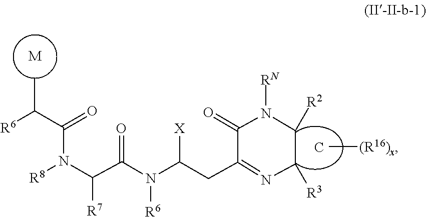

or a pharmaceutically acceptable salt, stereoisomer, solvate, prodrug, or tautomer thereof.

In some embodiments, the compound is of Formula (II′-II-b-2):

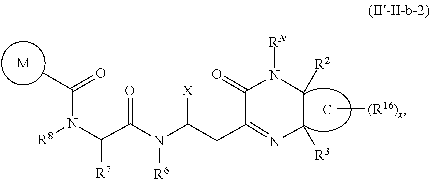

or a pharmaceutically acceptable salt, stereoisomer, solvate, prodrug, or tautomer thereof.

In some embodiments, the compound of Formula (A) is a compound of Formula (I).

In some embodiments, the compound of Formula (A) is a compound of Formula (II).

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

In some embodiments, the compound is selected from the compounds described in Table 3 and pharmaceutically acceptable salts, stereoisomers, solvates, prodrugs, isotopic derivatives, or tautomers thereof.

In some embodiments, the compound is selected from the compounds described in Table 3 and prodrugs and pharmaceutically acceptable salts thereof.

In some embodiments, the compound is selected from the compounds described in Table 3 and pharmaceutically acceptable salts thereof.

In some embodiments, the compound is selected from the prodrugs of the compounds described in Table 3 and pharmaceutically acceptable salts thereof.

In some embodiments, the compound is selected from the compounds described in Table 3.

TABLE 3
Certain examples of the compound of Formula (A)

| # | IUPAC Name |
|---|---|
| 101 | (1S,2S,5S)-N-[1-cyano-2-(2-oxo-1H-pyridin-3-yl)ethyl]-3-[(2S)-3,3-dimethyl- |
| | 2-[(2,2,2-trifluoroacetyl)amino]butanoyl]-6,6-dimethyl-3- |
| | azabicyclo[3.1.0]hexane-2-carboxamide |
| 102 | (1S,2S,5S)-N-[1-cyano-2-(6-methyl-2-oxo-1H-quinolin-3-yl)ethyl]-3-[(2S)-2- |
| | (methanesulfonamido)-3,3-dimethyl-butanoyl]-6,6-dimethyl-3- |
| | azabicyclo[3.1.0]hexane-2-carboxamide |
| 103 | (1S,2S,5S)-N-[1-cyano-2-(3-fluoro-2,6-dioxo-3H-pyridin-5-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 104 | (1S,2S,5S)-N-[2-(6-chloro-2-oxo-1H-quinolin-3-yl)-1-cyano-ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 105 | (1S,2S,5S)-N-[2-(7-chloro-2-oxo-1H-quinolin-3-yl)-1-cyano-ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 106 | (1S,2S,5S)-N-[1-cyano-2-(7-fluoro-2-oxo-1H-quinolin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 107 | (1S,2S,5S)-N-[(1S)-1-cyano-2-(6-methyl-2-oxo-1H-quinolin-3-yl)ethyl]-3- |
| | [(2S)-3,3-dimethyl-2-[(2,2,2-trifluoroacetyl)amino]butanoyl]-6,6-dimethyl-3- |
| | azabicyclo[3.1.0]hexane-2-carboxamide |
| 108 | (1S,2S,5S)-N-[2-amino-1-[(7-fluoro-2-oxo-1H-quinolin-3-yl)methyl]-2-oxo- |
| | ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 109 | (1S,2S,5S)-N-[1-cyano-2-(6-methoxy-2-oxo-1H-quinolin-3-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 110 | (1S,2S,5S)-N-[2-amino-1-[(6-methyl-2-oxo-1H-quinolin-3-yl)methyl]-2-oxo- |
| | ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 111 | (1S,2S,5S)-N-[2-amino-1-[(6-methoxy-2-oxo-1H-quinolin-3-yl)methyl]-2-oxo- |
| | ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 112 | (1S,2S,5S)-N-[1-cyano-2-(1-methyl-2-oxo-3-pyridyl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 113 | (1S,2S,5S)-N-[(1S)-1-cyano-2-(6-methyl-2-oxo-1H-quinolin-3-yl)ethyl]-3- |
| | [(2S)-3,3-dimethyl-2-[(2,2,2-trifluoroacetyl)amino]butanoyl]-6,6-dimethyl-3- |
| | azabicyclo[3.1.0]hexane-2-carboxamide |
| 114 | (1S,2S,5S)-N-[(1R)-1-cyano-2-(6-methyl-2-oxo-1H-quinolin-3-yl)ethyl]-3- |
| | [(2S)-3,3-dimethyl-2-[(2,2,2-trifluoroacetyl)amino]butanoyl]-6,6-dimethyl-3- |
| | azabicyclo[3.1.0]hexane-2-carboxamide |
| 115 | (1S,2S,5S)-N-[(1R)-1-cyano-2-[(3R)-6-methyl-2-oxo-3,4-dihydro-1H-quinolin- |
| | 3-yl]ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 116 | (1S,2S,5S)-N-[(1R)-1-cyano-2-[(3S)-6-methyl-2-oxo-3,4-dihydro-1H-quinolin- |
| | 3-yl]ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 117 | (1S,2S,5S)-N-[(1S)-1-cyano-2-[(3R)-6-methyl-2-oxo-3,4-dihydro-1H-quinolin- |
| | 3-yl]ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 118 | (1S,2S,5S)-N-[(1S)-1-cyano-2-[(3S)-6-methyl-2-oxo-3,4-dihydro-1H-quinolin- |
| | 3-yl]ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 119 | (1S,2S,5S)-N-[(1R)-1-cyano-2-(6-methyl-2-oxo-3,4-dihydro-1H-quinolin-3- |
| | yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 120 | (1S,2S,5S)-N-[(1S)-1-cyano-2-(6-methyl-2-oxo-3,4-dihydro-1H-quinolin-3- |
| | yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 121 | (1S,2S,5S)-N-[1-cyano-2-(2-oxo-5,6,7,8-tetrahydro-1H-quinolin-3-yl)ethyl]-3- |
| | [(2S)-3,3-dimethyl-2-[(2,2,2-trifluoroacetyl)amino]butanoyl]-6,6-dimethyl-3- |
| | azabicyclo[3.1.0]hexane-2-carboxamide |
| 122 | (1S,2S,5S)-N-[1-cyano-2-(2-oxo-5,6,7,8-tetrahydro-1H-quinolin-3-yl)ethyl]-3- |
| | [(2S)-2-(methanesulfonamido)-3,3-dimethyl-butanoyl]-6,6-dimethyl-3- |
| | azabicyclo[3.1.0]hexane-2-carboxamide |
| 123 | (1S,2S,5S)-N-[1-cyano-2-(2-oxo-1,5,7,8-tetrahydropyrano[4,3-b]pyridin-3- |
| | yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 124 | (1S,2S,5S)-N-[1-cyano-2-(2-oxo-1,5,7,8-tetrahydropyrano[4,3-b]pyridin-3- |
| | yl)ethyl]-3-[(2S)-2-(methanesulfonamido)-3,3-dimethyl-butanoyl]-6,6- |
| | dimethyl-3-azabicyclo[3.1.0]hexane-2-carboxamide |
| 125 | (1S,2S,5S)-N-[1-cyano-2-(6,6-difluoro-2-oxo-1,5,7,8-tetrahydroquinolin-3- |
| | yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 126 | (1S,2S,5S)-N-[1-cyano-2-(6,6-difluoro-2-oxo-1,5,7,8-tetrahydroquinolin-3- |
| | yl)ethyl]-3-[(2S)-2-(methanesulfonamido)-3,3-dimethyl-butanoyl]-6,6- |
| | dimethyl-3-azabicyclo[3.1.0]hexane-2-carboxamide |
| 127 | (1S,2S,5S)-N-[1-cyano-2-(6,6-dimethyl-2-oxo-1,5,7,8-tetrahydroquinolin-3- |
| | yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 128 | (1S,2S,5S)-N-[1-cyano-2-(6,6-dimethyl-2-oxo-1,5,7,8-tetrahydroquinolin-3- |
| | yl)ethyl]-3-[(2S)-2-(methanesulfonamido)-3,3-dimethyl-butanoyl]-6,6- |
| | dimethyl-3-azabicyclo[3.1.0]hexane-2-carboxamide |
| 129 | (1S,2S,5S)-N-[1-cyano-2-(2-oxo-1H-1,6-naphthyridin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 130 | (1S,2S,5S)-N-[1-cyano-2-(2-oxo-1H-1,6-naphthyridin-3-yl)ethyl]-3-[(2S)-2- |
| | (methanesulfonamido)-3,3-dimethyl-butanoyl]-6,6-dimethyl-3- |
| | azabicyclo[3.1.0]hexane-2-carboxamide |
| 131 | (1S,2S,5S)-N-[1-cyano-2-(6-methyl-2-oxo-1,5,7,8-tetrahydro-1,6-naphthyridin- |
| | 3-yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 132 | (1S,2S,5S)-N-[1-cyano-2-(6-methyl-2-oxo-1,5,7,8-tetrahydro-1,6-naphthyridin- |
| | 3-yl)ethyl]-3-[(2S)-2-(methanesulfonamido)-3,3-dimethyl-butanoyl]-6,6- |
| | dimethyl-3-azabicyclo[3.1.0]hexane-2-carboxamide |
| 133 | (1R,2S,5S)-N-[1-cyano-2-(6-isopropyl-2-oxo-1,5,7,8-tetrahydro-1,6- |
| | naphthyridin-3-yl)ethyl]-3-[(2S)-3,3-dimethyl-2-[(2,2,2- |
| | trifluoroacetyl)amino]butanoyl]-6,6-dimethyl-3-azabicyclo[3.1.0]hexane-2- |
| | carboxamide |
| 134 | (1R,2S,5S)-N-[1-cyano-2-(6-isopropyl-2-oxo-1,5,7,8-tetrahydro-1,6- |
| | naphthyridin-3-yl)ethyl]-3-[(2S)-2-(methanesulfonamido)-3,3-dimethyl- |
| | butanoyl]-6,6-dimethyl-3-azabicyclo[3.1.0]hexane-2-carboxamide |
| 135 | (1S,2S,5S)-N-[2-(6-acetyl-2-oxo-1,5,7,8-tetrahydro-1,6-naphthyridin-3-yl)-1- |
| | cyano-ethyl]-3-[(2S)-3,3-dimethyl-2-[(2,2,2-trifluoroacetyl)amino]butanoyl]- |
| | 6,6-dimethyl-3-azabicyclo[3.1.0]hexane-2-carboxamide |
| 136 | (1S,2S,5S)-N-[2-(6-acetyl-2-oxo-1,5,7,8-tetrahydro-1,6-naphthyridin-3-yl)-1- |
| | cyano-ethyl]-3-[(2S)-2-(methanesulfonamido)-3,3-dimethyl-butanoyl]-6,6- |
| | dimethyl-3-azabicyclo[3.1.0]hexane-2-carboxamide |
| 137 | (1S,2S,5S)-N-[1-cyano-2-(2-oxo-1H-1,7-naphthyridin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 138 | (1S,2S,5S)-N-[1-cyano-2-(2-oxo-1H-1,7-naphthyridin-3-yl)ethyl]-3-[(2S)-2- |
| | (methanesulfonamido)-3,3-dimethyl-butanoyl]-6,6-dimethyl-3- |
| | azabicyclo[3.1.0]hexane-2-carboxamide |
| 139 | (1S,2S,5S)-N-[1-cyano-2-(7-methyl-2-oxo-1,5,6,8-tetrahydro-1,7-naphthyridin- |
| | 3-yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 140 | (1S,2S,5S)-N-[1-cyano-2-(7-methyl-2-oxo-1,5,6,8-tetrahydro-1,7-naphthyridin- |
| | 3-yl)ethyl]-3-[(2S)-2-(methanesulfonamido)-3,3-dimethyl-butanoyl]-6,6- |
| | dimethyl-3-azabicyclo[3.1.0]hexane-2-carboxamide |
| 141 | (1R,2S,5S)-N-[1-cyano-2-(7-isopropyl-2-oxo-1,5,6,8-tetrahydro-1,7- |
| | naphthyridin-3-yl)ethyl]-3-[(2S)-3,3-dimethyl-2-[(2,2,2- |
| | trifluoroacetyl)amino]butanoyl]-6,6-dimethyl-3-azabicyclo[3.1.0]hexane-2- |
| | carboxamide |
| 142 | (1R,2S,5S)-N-[1-cyano-2-(7-isopropyl-2-oxo-1,5,6,8-tetrahydro-1,7- |
| | naphthyridin-3-yl)ethyl]-3-[(2S)-2-(methanesulfonamido)-3,3-dimethyl- |
| | butanoyl]-6,6-dimethyl-3-azabicyclo[3.1.0]hexane-2-carboxamide |
| 143 | (1S,2S,5S)-N-[2-(7-acetyl-2-oxo-1,5,6,8-tetrahydro-1,7-naphthyridin-3-yl)-1- |
| | cyano-ethyl]-3-[(2S)-3,3-dimethyl-2-[(2,2,2-trifluoroacetyl)amino]butanoyl]- |
| | 6,6-dimethyl-3-azabicyclo[3.1.0]hexane-2-carboxamide |
| 144 | (1S,2S,5S)-N-[2-(7-acetyl-2-oxo-1,5,6,8-tetrahydro-1,7-naphthyridin-3-yl)-1- |
| | cyano-ethyl]-3-[(2S)-2-(methanesulfonamido)-3,3-dimethyl-butanoyl]-6,6- |
| | dimethyl-3-azabicyclo[3.1.0]hexane-2-carboxamide |
| 145 | (1S,2S,5S)-N-[1-cyano-2-(6,7-dimethyl-2-oxo-1H-quinolin-3-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 146 | (1S,2S,5S)-N-[1-cyano-2-(7-methyl-2-oxo-1H-quinolin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 147 | (1S,2S,5S)-N-[(1S)-1-cyano-2-(6-methyl-2-oxo-1H-quinolin-3-yl)ethyl]-3- |
| | [(2S)-2-(methanesulfonamido)-3,3-dimethyl-butanoyl]-6,6-dimethyl-3- |
| | azabicyclo[3.1.0]hexane-2-carboxamide |
| 148 | (1S,2S,5S)-N-[(1R)-1-cyano-2-(6-methyl-2-oxo-1H-quinolin-3-yl)ethyl]-3- |
| | [(2S)-2-(methanesulfonamido)-3,3-dimethyl-butanoyl]-6,6-dimethyl-3- |
| | azabicyclo[3.1.0]hexane-2-carboxamide |
| 149 | (2S)-N-[1-cyano-2-(6-methyl-2-oxo-1H-quinolin-3-yl)ethyl]-1-[(2S)-3,3- |
| | dimethyl-2-[(2,2,2-trifluoroacetyl)amino]butanoyl]-4,4-dimethyl-piperidine-2- |
| | carboxamide |
| 150 | (1S,2S,5S)-N-[1-cyano-2-(6-methyl-2-oxo-1H-quinolin-3-yl)ethyl]-3-[(2R)-3,3- |
| | difluoro-2-[(2,2,2-trifluoroacetyl)amino]butanoyl]-6,6-dimethyl-3- |
| | azabicyclo[3.1.0]hexane-2-carboxamide |
| 151 | (1S,2S,5S)-N-[1-cyano-2-(6-methyl-2-oxo-1H-quinolin-3-yl)ethyl]-3-[(2S)-3,3- |
| | dimethyl-2-[(4-methylthiazol-2-yl)amino]butanoyl]-6,6-dimethyl-3- |
| | azabicyclo[3.1.0]hexane-2-carboxamide |
| 152 | (1S,2S,5S)-N-[1-cyano-2-(6-methyl-2-oxo-1H-quinolin-3-yl)ethyl]-3-[(2S)-3,3- |
| | dimethyl-2-[(5-methylthiazol-2-yl)amino]butanoyl]-6,6-dimethyl-3- |
| | azabicyclo[3.1.0]hexane-2-carboxamide |
| 153 | (1S,2S,5S)-N-[1-cyano-2-(6-methyl-2-oxo-1H-quinolin-3-yl)ethyl]-3-[(2S)-3,3- |
| | dimethyl-2-[(2-methylpyrimidin-4-yl)amino]butanoyl]-6,6-dimethyl-3- |
| | azabicyclo[3.1.0]hexane-2-carboxamide |
| 154 | (1S,2S,5S)-N-[1-cyano-2-(6-methyl-2-oxo-1H-quinolin-3-yl)ethyl]-3-[(2S)-3,3- |
| | dimethyl-2-[(4-methylpyrimidin-2-yl)amino]butanoyl]-6,6-dimethyl-3- |
| | azabicyclo[3.1.0]hexane-2-carboxamide |
| 155 | (1S,2S,5S)-N-[1-cyano-2-(6-methyl-2-oxo-1H-quinolin-3-yl)ethyl]-3-[(2S)-3,3- |
| | dimethyl-2-[2-(trifluoromethyl)pyrimidin-4-yl]amino]butanoyl]-6,6-dimethyl- |
| | 3-azabicyclo[3.1.0]hexane-2-carboxamide |
| 156 | (1S,2S,5S)-N-[1-cyano-2-(6-methyl-2-oxo-1H-quinolin-3-yl)ethyl]-3-[(2S)-3,3- |
| | dimethyl-2-[[4-(trifluoromethyl)pyrimidin-2-yl]amino]butanoyl]-6,6-dimethyl- |
| | 3-azabicyclo[3.1.0]hexane-2-carboxamide |
| 157 | (1S,2S,5S)-N-[(1S)-1-cyano-2-(6-methyl-2-oxo-1H-quinolin-3-yl)ethyl]-3- |
| | [(2S)-3,3-dimethyl-2-[(4-methylthiazol-2-yl)amino]butanoyl]-6,6-dimethyl-3- |
| | azabicyclo[3.1.0]hexane-2-carboxamide |
| 158 | (1S,2S,5S)-N-[(1S)-1-cyano-2-(6-methyl-2-oxo-1H-quinolin-3-yl)ethyl]-3- |
| | [(2S)-3,3-dimethyl-2-[(5-methylthiazol-2-yl)amino]butanoyl]-6,6-dimethyl-3- |
| | azabicyclo[3.1.0]hexane-2-carboxamide |
| 159 | (1S,2S,5S)-N-[(1S)-1-cyano-2-(6-methyl-2-oxo-1H-quinolin-3-yl)ethyl]-3- |
| | [(2S)-3,3-dimethyl-2-[(2-methylpyrimidin-4-yl)amino]butanoyl]-6,6-dimethyl- |
| | 3-azabicyclo[3.1.0]hexane-2-carboxamide |
| 160 | (1S,2S,5S)-N-[(1S)-1-cyano-2-(6-methyl-2-oxo-1H-quinolin-3-yl)ethyl]-3- |
| | [(2S)-3,3-dimethyl-2-[(4-methylpyrimidin-2-yl)amino]butanoyl]-6,6-dimethyl- |
| | 3-azabicyclo[3.1.0]hexane-2-carboxamide |
| 161 | (1S,2S,5S)-N-[(1S)-1-cyano-2-(6-methyl-2-oxo-1H-quinolin-3-yl)ethyl]-3- |
| | [(2S)-3,3-dimethyl-2-[2-(trifluoromethyl)pyrimidin-4-yl]amino]butanoyl]-6,6- |
| | dimethyl-3-azabicyclo[3.1.0]hexane-2-carboxamide |
| 162 | (1S,2S,5S)-N-[(1S)-1-cyano-2-(6-methyl-2-oxo-1H-quinolin-3-yl)ethyl]-3- |
| | [(2S)-3,3-dimethyl-2-[[4-(trifluoromethyl)pyrimidin-2-yl]amino]butanoyl]-6,6- |
| | dimethyl-3-azabicyclo[3.1.0]hexane-2-carboxamide |
| 163 | (1S,2S,5S)-N-[1-cyano-2-(6-methyl-2-oxo-1H-quinolin-3-yl)ethyl]-3-[(2S)-3,3- |
| | dimethyl-2-(2,2,2-trifluoroethylsulfonylamino)butanoyl]-6,6-dimethyl-3- |
| | azabicyclo[3.1.0]hexane-2-carboxamide |
| 164 | (2S)-N-[(1S)-1-benzyl-2-[[1-cyano-2-(6-methyl-2-oxo-1H-quinolin-3- |
| | yl)ethyl]amino]-2-oxo-ethyl]-3,3-dimethyl-2-(2,2,2- |
| | trifluoroethylsulfonylamino)butanamide |
| 165 | (2S)-N-[(1S)-1-benzyl-2-[[1-cyano-2-(6-methyl-2-oxo-1H-quinolin-3- |
| | yl)ethyl]amino]-2-oxo-ethyl]-N,3,3-trimethyl-2-(2,2,2- |
| | trifluoroethylsulfonylamino)butanamide |
| 166 | (2S)-N-[(1S)-2-[[1-cyano-2-(6-methyl-2-oxo-1H-quinolin-3-yl)ethyl]amino]-2- |
| | oxo-1-[(2,4,5-trifluorophenyl)methyl]ethyl]-3,3-dimethyl-2-(2,2,2- |
| | trifluoroethylsulfonylamino)butanamide |
| 167 | (2S)-N-[(1S)-2-[[1-cyano-2-(6-methyl-2-oxo-1H-quinolin-3-yl)ethyl]amino]-2- |
| | oxo-1-[(2,4,5-trifluorophenyl)methyl]ethyl]-N,3,3-trimethyl-2-(2,2,2- |
| | trifluoroethylsulfonylamino)butanamide |
| 168 | (2S)-N-[(1S)-2-[[1-cyano-2-(6-methyl-2-oxo-1H-quinolin-3-yl)ethyl]-methyl- |
| | amino]-2-oxo-1-[(2,4,5-trifluorophenyl)methyl]ethyl]-N,3,3-trimethyl-2-(2,2,2- |
| | trifluoroethylsulfonylamino)butanamide |
| 169 | (1S,2S,5S)-N-[1-cyano-2-(6-methyl-2-oxo-1H-quinolin-3-yl)ethyl]-3-[(2S)-2- |
| | (methanesulfonamido)-3,3-dimethyl-butanoyl]-N,6,6-trimethyl-3- |
| | azabicyclo[3.1.0]hexane-2-carboxamide |
| 170 | (1R,2S,5S)-N-[1-cyano-2-(6-methyl-2-oxo-1H-quinolin-3-yl)ethyl]-N-ethyl-3- |
| | [(2S)-2-(methanesulfonamido)-3,3-dimethyl-butanoyl]-6,6-dimethyl-3- |
| | azabicyclo[3.1.0]hexane-2-carboxamide |
| 171 | tert-butyl N-[(1S)-1-[(1S,2S,5S)-2-[1-cyano-2-(6-methyl-2-oxo-1H-quinolin-3- |
| | yl)ethyl]carbamoyl]-6,6-dimethyl-3-azabicyclo[3.1.0]hexane-3-carbonyl]-2,2- |
| | dimethyl-propyl]carbamate |
| 172 | (1R,2S,5S)-N-[(1S)-1-cyano-2-[(3S)-2,6-dioxo-3-piperidyl]ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 173 | (1R,2S,5S)-N-[(1R)-1-cyano-2-(2-oxo-1H-1,6-naphthyridin-3-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 174 | (1R,2S,5S)-N-[(1S)-1-cyano-2-(2-oxo-1H-1,6-naphthyridin-3-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 175 | (1R,2S,5S)-N-[(1S)-1-cyano-2-(7-methoxy-6-methyl-2-oxo-1H-quinolin-3- |
| | yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 176 | (1R,2S,5S)-N-[(1S)-1-cyano-2-(7-fluoro-6-methyl-2-oxo-1H-quinolin-3- |
| | yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 177 | (1R,2S,5S)-N-[(1S)-1-cyano-2-(6,7-difluoro-2-oxo-1H-quinolin-3-yl)ethyl]-3- |
| | [(2S)-3,3-dimethyl-2-[(2,2,2-trifluoroacetyl)amino]butanoyl]-6,6-dimethyl-3- |
| | azabicyclo[3.1.0]hexane-2-carboxamide |
| 178 | (1R,2S,5S)-N-[(1S)-1-cyano-2-(4,6-dimethyl-2-oxo-1H-quinolin-3-yl)ethyl]-3- |
| | [(2S)-3,3-dimethyl-2-[(2,2,2-trifluoroacetyl)amino]butanoyl]-6,6-dimethyl-3- |
| | azabicyclo[3.1.0]hexane-2-carboxamide |
| 179 | (1R,2S,5S)-N-[(1S)-1-cyano-2-(6-methoxy-7-methyl-2-oxo-1H-quinolin-3- |
| | yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 180 | (1R,2S,5S)-N-[(1S)-1-cyano-2-(7-methyl-2-oxo-1H-quinolin-3-yl)ethyl]-3- |
| | [(2S)-3,3-dimethyl-2-[(2,2,2-trifluoroacetyl)amino]butanoyl]-6,6-dimethyl-3- |
| | azabicyclo[3.1.0]hexane-2-carboxamide |
| 181 | (1R,2S,5S)-N-[(1S)-1-cyano-2-(6,7-dimethyl-2-oxo-1H-quinolin-3-yl)ethyl]-3- |
| | [(2S)-3,3-dimethyl-2-[(2,2,2-trifluoroacetyl)amino]butanoyl]-6,6-dimethyl-3- |
| | azabicyclo[3.1.0]hexane-2-carboxamide |
| 182 | (1R,2S,5S)-N-[1-cyano-2-(2-oxo-1H-quinolin-3-yl)ethyl]-3-[(2S)-3,3-dimethyl- |
| | 2-[(2,2,2-trifluoroacetyl)amino]butanoyl]-6,6-dimethyl-3- |
| | azabicyclo[3.1.0]hexane-2-carboxamide |
| 183 | (1R,2S,5S)-N-[(1S)-1-cyano-2-(6-methyl-2-oxo-1H-quinolin-3-yl)ethyl]-3- |
| | [(2S)-3,3-dimethyl-2-[(2,2,2-trifluoroacetyl)amino]butanoyl]-N,6,6-trimethyl-3- |
| | azabicyclo[3.1.0]hexane-2-carboxamide |
| 184 | N-[1-cyano-2-(6-methyl-2-oxo-1H-quinolin-3-yl)ethyl]-3-[3,3-dimethyl-2- |
| | [(2,2,2-trifluoroacetyl)amino]butanoyl]-N-ethyl-6,6-dimethyl-3- |
| | azabicyclo[3.1.0]hexane-2-carboxamide |
| 185 | (1S,2S,5S)-N-[1-cyano-2-(3-oxo-4H-quinoxalin-2-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 186 | (1S,2S,5S)-N-[1-cyano-2-(3-oxo-4H-quinoxalin-2-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 187 | (1S,2S,5S)-N-[1-cyano-2-(1-methyl-3-oxo-2,4-dihydroquinoxalin-2-yl)ethyl]-3- |
| | [(2S)-3,3-dimethyl-2-[(2,2,2-trifluoroacetyl)amino]butanoyl]-6,6-dimethyl-3- |
| | azabicyclo[3.1.0]hexane-2-carboxamide |
| 188 | (1S,2S,5S)-N-[(1S)-1-cyano-2-(2-oxo-1H-1,6-naphthyridin-3-yl)ethyl]-3-[(2S)- |
| | 2-(methanesulfonamido)-3,3-dimethyl-butanoyl]-6,6-dimethyl-3- |
| | azabicyclo[3.1.0]hexane-2-carboxamide |
| 189 | (1S,2S,5S)-N-[(1R)-1-cyano-2-(2-oxo-1H-1,6-naphthyridin-3-yl)ethyl]-3-[(2S)- |
| | 2-(methanesulfonamido)-3,3-dimethyl-butanoyl]-6,6-dimethyl-3- |
| | azabicyclo[3.1.0]hexane-2-carboxamide |
| 190 | (1R,2S,5S)-N-[(1S)-1-cyano-2-(6-methyl-2-oxo-1H-quinolin-3-yl)ethyl]-6,6- |
| | dimethyl-3-[2-(3-methyl-1H-pyrazol-5-yl)acetyl]-3-azabicyclo[3.1.0]hexane-2- |
| | carboxamide |
| 191 | (1R,2S,5S)-N-[(1S)-1-cyano-2-(6-methyl-2-oxo-1H-quinolin-3-yl)ethyl]-6,6- |
| | dimethyl-3-[2-[3-(trifluoromethyl)-1H-pyrazol-5-yl]acetyl]-3- |
| | azabicyclo[3.1.0]hexane-2-carboxamide |
| 192 | (1R,2S,5S)-N-[(1S)-1-cyano-2-(6-methyl-2-oxo-1H-quinolin-3-yl)ethyl]-6,6- |
| | dimethyl-3-[2-(3-methylisoxazol-5-yl)acetyl]-3-azabicyclo[3.1.0]hexane-2- |
| | carboxamide |
| 193 | (1R,2S,5S)-N-[(1S)-1-cyano-2-(6-methyl-2-oxo-1H-quinolin-3-yl)ethyl]-6,6- |
| | dimethyl-3-[2-[3-(trifluoromethyl)isoxazol-5-yl]acetyl]-3- |
| | azabicyclo[3.1.0]hexane-2-carboxamide |
| 194 | (1S,2S,5S)-N-[(1S)-1-cyano-2-(6-methyl-2-oxo-1H-quinolin-3-yl)ethyl]-6,6- |
| | dimethyl-3-[2-[3-(trifluoromethyl)-1H-pyrazol-5-yl]propanoyl]-3- |
| | azabicyclo[3.1.0]hexane-2-carboxamide |
| 195 | (1S,2S,5S)-N-[(1S)-1-cyano-2-(6-methyl-2-oxo-1H-quinolin-3-yl)ethyl]-3- |
| | [(2S)-3,3-dimethyl-2-(2,2,2-trifluoroethylsulfonylamino)butanoyl]-6,6- |
| | dimethyl-3-azabicyclo[3.1.0]hexane-2-carboxamide |
| 196 | (1R,2S,5S)-N-[(1S)-1-cyano-2-(6-methyl-2-oxo-1H-quinolin-3-yl)ethyl]-3-(4- |
| | methoxybenzofuran-2-carbonyl)-6,6-dimethyl-3-azabicyclo[3.1.0]hexane-2- |
| | carboxamide |
| 197 | benzyl N-[(1S)-1-[(3aR,6aR)-5-[[(1S)-1-cyano-2-(6-methyl-2-oxo-1H-quinolin- |
| | 3-yl)ethyl]carbamoyl]-2,3,3a,5,6,6a-hexahydrofuro[3,2-b]pyrrole-4-carbonyl]- |
| | 2,2-dimethyl-propyl]carbamate |
| 198 | (3aR,6aR)-N-[(1S)-1-cyano-2-(6-methyl-2-oxo-1H-quinolin-3-yl)ethyl]-4- |
| | [(2S)-3,3-dimethyl-2-[(2,2,2-trifluoroacetyl)amino]butanoyl]-2,3,3a,5,6,6a- |
| | hexahydrofuro[3,2-b]pyrrole-5-carboxamide |
| 199 | (1S,2S,5S)-N-[1-cyano-2-(6-methyl-2-oxo-3,4-dihydro-1H-quinolin-3- |
| | yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 200 | methyl N-[(1R)-1-[(1S)-2-[(1R)-1-cyano-2-(6-methyl-2-oxo-1H-quinolin-3- |
| | yl)ethyl]carbamoyl]-6,6-dimethyl-3-azabicyclo[3.1.0]hexane-3-carbonyl]-2,2- |
| | dimethyl-propyl]carbamate |
| 201 | ethyl N-[(1R)-1-[(1S)-2-[[(1R)-1-cyano-2-(6-methyl-2-oxo-1H-quinolin-3- |
| | yl)ethyl]carbamoyl]-6,6-dimethyl-3-azabicyclo[3.1.0]hexane-3-carbonyl]-2,2- |
| | dimethyl-propyl]carbamate |
| 202 | isopropyl N-[(1R)-1-[(1S)-2-[[(1R)-1-cyano-2-(6-methyl-2-oxo-1H-quinolin-3- |
| | yl)ethyl]carbamoyl]-6,6-dimethyl-3-azabicyclo[3.1.0]hexane-3-carbonyl]-2,2- |
| | dimethyl-propyl]carbamate |
| 203 | (1S)-N-[(1R)-1-cyano-2-(6-methyl-2-oxo-1H-quinolin-3-yl)ethyl]-3-[(2R)-2- |
| | (cyclopropylsulfonylamino)-3,3-dimethyl-butanoyl]-6,6-dimethyl-3- |
| | azabicyclo[3.1.0]hexane-2-carboxamide |
| 204 | (1S)-N-[(1R)-1-cyano-2-(6-methyl-2-oxo-1H-quinolin-3-yl)ethyl]-3-[(2R)-3,3- |
| | dimethyl-2-(2-methylpropanoylamino)butanoyl]-6,6-dimethyl-3- |
| | azabicyclo[3.1.0]hexane-2-carboxamide |
| 205 | (1S,2S,5S)-N-[1-cyano-2-(6-methyl-2-oxo-1H-quinolin-3-yl)ethyl]-3-[(2S)-3,3- |
| | dimethyl-2-[(2,2,2-trifluoroacetyl)amino]butanoyl]-N,6,6-trimethyl-3- |
| | azabicyclo[3.1.0]hexane-2-carboxamide |
| 206 | (2S)-N-[(1S)-1-benzyl-2-[[(1S)-1-cyano-2-(6-methyl-2-oxo-1H-quinolin-3- |
| | yl)ethyl]amino]-2-oxo-ethyl]-N,3,3-trimethyl-2-[(2,2,2- |
| | trifluoroacetyl)amino]butanamide |
| 207 | (2S)-N-[(1S)-1-benzyl-2-[[(1R)-1-cyano-2-(6-methyl-2-oxo-1H-quinolin-3- |
| | yl)ethyl]amino]-2-oxo-ethyl]-N,3,3-trimethyl-2-[(2,2,2- |
| | trifluoroacetyl)amino]butanamide |
| 208 | (1R,2S,5S)-N-[(1R)-1-cyano-2-(6-methyl-2-oxo-1H-quinolin-3-yl)ethyl]-3- |
| | [(2S)-3,3-dimethyl-2-[(2,2,2-trifluoroacetyl)amino]butanoyl]-N,6,6-trimethyl-3- |
| | azabicyclo[3.1.0]hexane-2-carboxamide |
| 209 | (1R,2S,5S)-N-[(1S)-1-cyano-2-(6-methyl-2-oxo-1H-quinolin-3-yl)ethyl]-3- |
| | [(2S)-3,3-dimethyl-2-[(2,2,2-trifluoroacetyl)amino]butanoyl]-N,6,6-trimethyl-3- |
| | azabicyclo[3.1.0]hexane-2-carboxamide |

In some embodiments, the compound is a pharmaceutically acceptable salt of any one of the compounds described in Table 3.

In some embodiments, the compound is a lithium salt, sodium salt, potassium salt, calcium salt, or magnesium salt of any one of the compounds described in Table 3.

In some embodiments, the compound is a sodium salt or potassium salt of any one of the compounds described in Table 3.

In some embodiments, the compound is a salt of any acid described in the Table 4 and any one of the compounds described in Table 3.

TABLE 4
Pharmaceutically acceptable acid forming
salts with the Compound of Formula (A).

| | | |
|---|---|---|
| 1-hydroxy-2-naphthoic acid | ethanesulfonic acid | methanesulfonic acid |
| 2,2-dichloroacetic acid | formic acid | naphthalene-1,5-disulfonic |
| 2-hydroxyethanesulfonic | fumaric acid | acid |
| acid | galactaric acid | naphthalene-2-sulfonic acid |
| 2-oxoglutaric acid | gentisic acid | nicotinic acid |
| 4-acetamidobenzoic acid | glucoheptonic acid (D) | nitric acid |
| 4-aminosalicylic acid | gluconic acid (D) | oleic acid |
| acetic acid | glucuronic acid (D) | oxalic acid |
| adipic acid | glutamic acid | palmitic acid |
| ascorbic acid (L) | glutaric acid | pamoic acid |
| aspartic acid (L) | glycerophosphoric acid | phosphoric acid |
| benzenesulfonic acid | glycolic acid | proprionic acid |
| benzoic acid | hippuric acid | pyroglutamic acid (−L) |
| camphoric acid (+) | hydrobromic acid | salicylic acid |
| camphor-10-sulfonic acid | hydrochloric acid | sebacic acid |
| (+) | isobutyric acid | stearic acid |
| capric acid (decanoic acid) | lactic acid (DL) | succinic acid |
| caproic acid (hexanoic acid) | lactobionic acid | sulfuric acid |
| caprylic acid (octanoic acid) | lauric acid | tartaric acid (+L) |
| carbonic acid | maleic acid | thiocyanic acid |
| cinnamic acid | malic acid (−L) | toluenesulfonic acid (p) |
| citric acid | malonic acid | undecylenic acid |
| cyclamic acid | mandelic acid (DL) | |
| dodecylsulfuric acid | | |
| ethane-1,2-disulfonic acid | | |

In some embodiments, the compound is a salt of acetic acid and any one of the compounds described in Table 3.

In some embodiments, the compound is a salt of adipic acid and any one of the compounds described in Table 3.

In some embodiments, the compound is a salt of ascorbic acid (L) and any one of the compounds described in Table 3.

In some embodiments, the compound is a salt of hydrobromic acid and any one of the compounds described in Table 3.

In some embodiments, the compound is a salt of hydrochloric acid and any one of the compounds described in Table 3.

In some embodiments, the compound is a salt of citric acid and any one of the compounds described in Table 3.

In some embodiments, the compound is a salt of glutamic acid and any one of the compounds described in Table 3.

In some embodiments, the compound is a salt of oxalic acid and any one of the compounds described in Table 3.

In some embodiments, the compound is a salt of formic acid and any one of the compounds described in Table 3.

In some embodiments, the compound is a salt of sulfuric acid and any one of the compounds described in Table 3.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 3 and prodrugs and pharmaceutically acceptable salts thereof.

In some embodiments, the compound is an isotopic derivative of any one of the compounds described in Table 3 and pharmaceutically acceptable salts thereof.

In some embodiments, the compound is an isotopic derivative of any one of prodrugs of the compounds described in Table 3 and pharmaceutically acceptable salts thereof.

In some embodiments, the compound is an isotopic derivative of any one of the compounds described in Table 3.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (A) is isotopically enriched with regard to, or labelled with, one or more isotopes as compared to the corresponding compound of Formula (A). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof).

In some embodiments, the compound is a deuterium labeled compound of any one of the compounds described in Table 3 and prodrugs and pharmaceutically acceptable salts thereof.

In some embodiments, the compound is a deuterium labeled compound of any one of the compounds described in Table 3 and pharmaceutically acceptable salts thereof.

In some embodiments, the compound is a deuterium labeled compound of any one of the prodrugs of the compounds described in Table 3 and pharmaceutically acceptable salts thereof.

In some embodiments, the compound is a deuterium labeled compound of any one of the compounds described in Table 3.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the disclosure or a pharmaceutically acceptable salt or solvate thereof that contains the aforementioned deuterium atom(s) is within the scope of the disclosure.

Further, substitution with deuterium (i.e., ²H) may afford certain therapeutic advantages resulting from greater metabolic stability, e.g., increased in vivo half-life or reduced dosage requirements.

In some embodiments, the compound is a ¹⁸F labeled compound.

In some embodiments, the compound is a ³³S labeled compound, a ³⁴S labeled compound, a ³⁵S labeled compound, a ³⁶S labeled compound, or any combination thereof.

It is understood that the ¹⁸F, ³³S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ³³S, ³⁴S, ³⁵S, and/or ³⁶S labeled reagent for a non-isotope labeled reagent.

A compound of the disclosure or a pharmaceutically acceptable salt or solvate thereof that contains one or more of the aforementioned ¹⁸F, ³³S, ³⁴S, ³⁵S, and ³⁶S atom(s) is within the scope of the disclosure. Further, substitution with isotope (e.g., ¹⁸F, ³³S, ³⁴S, ³⁵S, and/or ³⁶S) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

The various functional groups and substituents making up the compounds of the Formula (A) are typically chosen such that the molecular weight of the compound does not exceed 1000 Daltons. More usually, the molecular weight of the compound will be less than 1000, for example less than 900, or less than 800, or less than 700, or less than 600, or less than 500.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compound of Formula (A) include all such forms. For the avoidance of doubt, where a compound can exist in one of several tautomeric forms, and only one is specifically described or shown, all others are nevertheless embraced by Formula (A). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

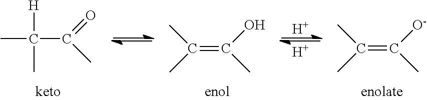

Compounds of any one of the Formulae disclosed herein containing an amine function may also form N-oxides. A reference herein to a compound of Formula (A) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄ alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

### Method of Synthesizing the Compounds

The compounds of the present invention may be made by a variety of methods, including standard chemistry. Suitable synthetic routes are depicted in the Schemes given below.

The compounds of Formula (A) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (A). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedures (General schemes I, II, III, IV) which comprise different sequences of assembling intermediates or compounds. Starting materials are either commercially available or made by known procedures in the reported literature or as illustrated below.

### General Procedure

In general, the compound of the Formula (A) can be prepared using reactions well known to those skilled in the art of organic synthesis. General scheme I showed possible synthetic route for the preparation of compound of the Formula (A) presented below:

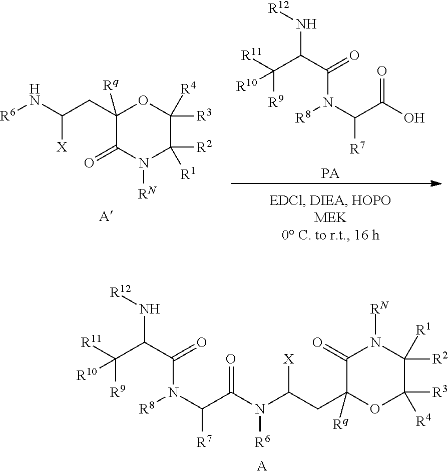

All reagents may be commercially available compounds itself or products of synthesis from commercially available reagents. For each compound in preparation may be used one step or multistep synthetic procedures, including but not limited procedures described herein in preparative part.

It should be obvious for specialist in this field that any of compound of Formula (I) obtained according to the procedures described above may be a subject for further transformation and modification that will led to other compound of Formula (I).

Any of described herein residue R¹² can be introduced into the structure of compound of Formula (I) using different approaches, some of them presented on the Scheme below.

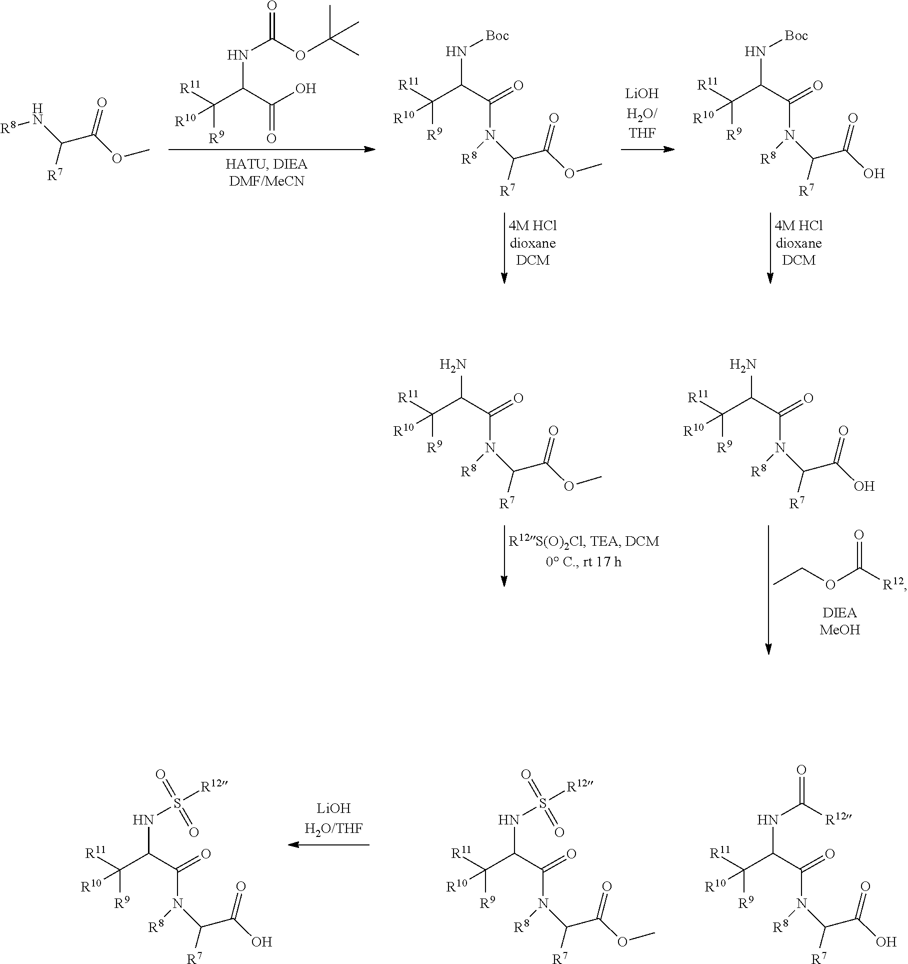

In some embodiments compound of Formula (A) can be subject for further transformations leading to other compound of Formula (A):

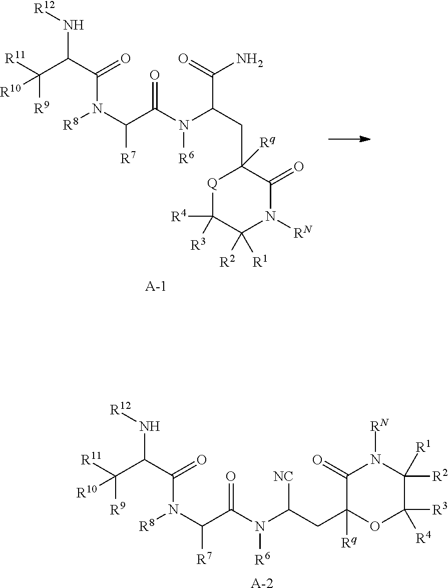

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 3.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-3-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

Additional therapeutic agents that can be used in the methods of the invention include Diosmin, Hesperidin, MK-3207, Venetoclax, Dihydroergocristine, Bolazine, R428, Ditercalinium, Etoposide, Teniposide, UK-432097, lrinotecan, Lumacaftor, Velpatasvir, Eluxadoline, Ledipasvir, Lopinavir/Ritonavir+Ribavirin, Alferon, and prednisone. Other additional agents useful in the methods of the present invention include dexamethasone, azithromycin and remdesivir as well as boceprevir, umifenovir and favipiravir.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure maybe obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a coronavirus infection, slow its progression and/or reduce the symptoms associated with the condition.

The size of the dose for therapeutic or prophylactic purposes of a compound of Formula (A) will naturally vary according to the nature and severity of the conditions, the age and sex of the animal or subject and the route of administration, according to well-known principles of medicine.

### Methods of Use

In some aspects, the present disclosure provides a method of inhibition of SARS-CoV-2-related 3C-like protease (e.g., in vitro or in vivo), comprising contacting a cell with a therapeutically effective amount of a compound of the present disclosure or a pharmaceutically acceptable salt thereof.

In some aspects, the present disclosure provides a method of treating or preventing a coronavirus infection in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a coronavirus infection in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with coronavirus. In some embodiments, the disease or disorder is a disease or disorder in which coronavirus is implicated.

The compounds of the invention are inhibitors of SARS-CoV-2-related 3C-like protease.

In some embodiments, the compounds, compositions, and methods disclosed herein are used in the prevention or treatment of a disease, disorder, or condition associated with coronavirus infection.

The compounds of the invention are also useful in treating diseases associated with the coronavirus infection. For example, diseases and conditions treatable according to the methods of the invention include COVID-19.

In some aspects, the present disclosure provides a method of treating or preventing a coronavirus infection in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a COVID-19 infection in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a coronavirus infection in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a COVID-19 in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting of SARS-CoV-2-related 3C-like protease (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a coronavirus infection in a subject in need thereof.

In some aspects, the present disclosure provides a compound of the present disclosure or a pharmaceutically acceptable salt thereof for use in treating or preventing a COVID-19 in a subject in need thereof.

In some aspects, the present disclosure provides use of a compound of the present disclosure or a pharmaceutically acceptable salt thereof in the manufacture of a medicament for inhibiting of SARS-CoV-2-related 3C-like protease (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing a coronavirus infection in a subject in need thereof.

In some aspects, the present disclosure provides use of a compound of the present disclosure or a pharmaceutically acceptable salt thereof in the manufacture of a medicament for treating or preventing a COVID-19 in a subject in need thereof.

The present disclosure provides compounds that function as inhibitors of SARS-CoV-2-related 3C-like protease (e.g., in vitro or in vivo). The present disclosure therefore provides a method of inhibiting of SARS-CoV-2-related 3C-like protease in vitro or in vivo, said method comprising contacting a cell with a therapeutically effective amount of a compound, or a pharmaceutically acceptable salt thereof, as defined herein.

In some embodiments, the inhibitors of SARS-CoV-2-related 3C-like protease is a compound of the present disclosure.

Effectiveness of compounds of the disclosure can be determined by industry-accepted assays/disease models according to standard practices of elucidating the same as described in the art and are found in the current general knowledge.

The present disclosure also provides a method of treating a disease or disorder in which coronavirus infection are implicated in a subject in need of such treatment, said method comprising administering to said subject a therapeutically effective amount of a compound, or a pharmaceutically acceptable salt thereof, or a pharmaceutical composition as defined herein.

In some embodiments, the subject is a mammal. In some embodiments, the subject is a human.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Abbreviations used in the following examples and elsewhere herein are:
ACN acetonitrile
AcOH acetic acid
AcCl acetyl chloride
anh. anhydrous
aq. aqueous
br. broad
Burgess reagent methyl N-(triethylammoniumsulfonyl)carbamate
calc. calculated
conc. concentrated
d duplet
DBU 1,8-diazabicyclo[5.4.0]undec-7-ene
DCM dichloromethane
DIPEA N,N-diisopropylethylamine
DMF dimethylformamide
DMSO dimethyl sulfoxide
DTT dithiothreitol
EDCI 1-ethyl-3-(3-dimethylaminopropyl)carbodiimide hydrochloride
ESI electrospray ionization
FC flash chromatography
h hour(s)
HATU 1-[Bis(dimethylamino)methylene]-1H-1,2,3-triazolo[4,5-b]pyridinium 3-oxid hexafluorophosphate
HPLC high pressure (or performance) liquid chromatography
HOPO 2-hydroxypyridine-N-oxide
IPA iso-propanol
LCMS liquid chromatography mass spectrometry
m multiplet
M molar
MEK methyl ethyl ketone
MHz megahertz
min minutes
Ms methanesulfonyl, mesyl
MsCl methanesulfonyl chloride
NMR nuclear magnetic resonance
q quadruplet
rt room temperature
s singlet
s solid
sat. saturated
t temperature, triplet
t_{R} retention time (in chromatography)
TEA triethylamine
TFA trifluoroacetic acid
THF tetrahydrofuran
TLC thin layer chromatography
TMEDA N,N,N′,N′-tetramethyl ethylenediamine

### EXAMPLES

### General Synthetical Procedures and Examples of the Compound's Preparation

### Synthesis of Building Blocks

### Synthesis of (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid (PA.4)

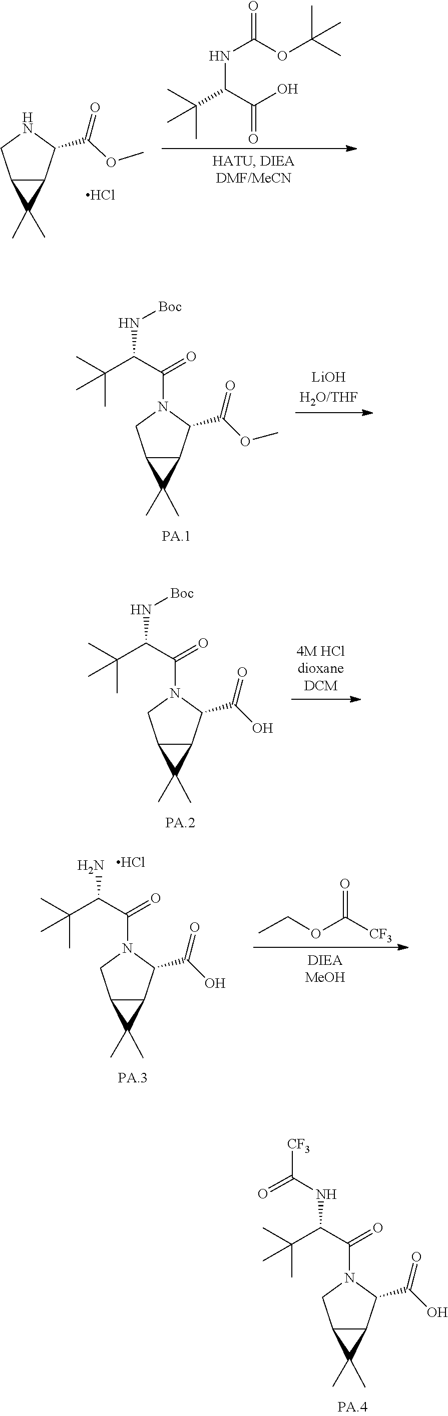

### Preparation 1. (1R,2S,5S)-3-((S)-2-((tert-Butoxycarbonyl)amino)-3,3-dimethylbutanoyl)-6,6-dimethyl-3-azabicyclo[3.1.0]hexane-2-carboxylate (PA.1)

To a solution of methyl (1R,2S,5S)-6,6-dimethyl-3-azabicyclo[3.1.0]hexane-2-carboxylate HCl salt (4.5 g, 21.9 mmol) and N-(tert-butoxycarbonyl)-3-methyl-L-valine (5.58 g, 24.1 mmol) in a mixture of N,N-dimethylformamide (9.0 ml) and acetonitrile (81 ml) was added HATU (9.09 g, 24.1 mmol) and N,N-diisopropylethylamine (8.55 g, 95.7 mmol) at 0° C. The reaction mixture was then allowed to warm to 25° C. and then stirred for 16 h. The mixture was treated with ethyl acetate, washed by water and 1M HCl_{(aq)}, and saturated aq. sodium chloride solution. The organic layer was dried over sodium sulfate, filtered, and concentrated. The residue was purified by silica gel chromatography (n-Hexane:ethyl acetate=3:1) to give the desired product PA.1 (5.6 g, 67%). ¹H NMR (400 MHz, DMSO-d₆), δ: 6.72 (d, J=9.6 Hz, 1H), 4.20 (s, 1H), 4.05-4.03 (m, 1H), 3.92 (d, J=10.4 Hz, 1H), 3.80-3.76 (m, 1H), 3.64 (s, 3H), 1.53-1.50 (m, 1H), 1.41-1.39 (m, 1H), 1.34 (s, 9H), 1.00 (s, 3H), 0.92 (s, 9H), 0.84 (s, 3H).

### Preparation 2. (1R,2S,5S)-3-((S)-2-((tert-Butoxycarbonyl)amino)-3,3-dimethylbutanoyl)-6,6-dimethyl-3-azabicyclo[3.1.0]hexane-2-carboxylic acid (PA.2)

To a solution of PA.1 (5.6 g, 15 mmol) in THF (20 ml) was added lithium hydroxide (1.1 g, 44 mmol) and water (20 ml). After the reaction mixture was stirred at 25° C. for 17 h, it was concentrated to remove THF. The residue was then adjusted to pH 2 by addition of 1 M HCl_{(aq)}. The resulting mixture was extracted with ethyl acetate, and the combined organic layers were washed with brine, dried over sodium sulfate, filtered, and concentrated to give PA.2 (6.16 g, crude). ¹H NMR (400 MHz, DMSO-d₆), δ: 6.67 (d, J=9.2 Hz, 1H), 4.11 (s, 1H), 4.05-4.03 (m, 1H), 3.90 (d, J=10.4 Hz, 1H), 3.78-3.74 (m, 1H), 1.50-1.47 (m, 1H), 1.39-1.38 (m, 1H), 1.34 (s, 9H), 1.00 (s, 3H), 0.92 (s, 9H), 0.83 (s, 3H).

### Preparation 3. (1R,2S,5S)-3-((S)-2-Amino-3,3-dimethylbutanoyl)-6,6-dimethyl-3-azabicyclo[3.1.0]hexane-2-carboxylic acid hydrochloride (PA.3)

A solution of 4M HCl in 1,4-dioxane (21 ml) was added to a solution of PA.2 (6.16 g) in DCM (19 ml). After the mixture was stirred at 25° C. for 16 h, the solvent was removed under reduced pressure to give a HCl salt of PA.3 (4.7 g, crude). ¹H NMR (400 MHz, DMSO-d₆), δ: 8.31 (br. s, 2H), 4.15 (s, 1H), 3.80-3.76 (m, 2H), 3.72-3.69 (m, 1H), 1.55-1.52 (m, 1H), 1.43 (d, J=7.6 Hz, 1H), 1.02 (s, 9H), 1.01 (s, 3H), 0.95 (s, 3H).

### Preparation 4. (1R,2S,5S)-3-((S)-3,3-Dimethyl-2-(2,2,2-trifluoroacetamido)butanoyl)-6,6-dimethyl-3-azabicyclo[3.1.0]hexane-2-carboxylic acid (PA.4)

To a solution of the HCl salt of PA.3 (4.7 g, crude) in methanol (15 ml) was added triethylamine (13 ml) and ethyl trifluoroacetate (3.13 mL, 26.3 mmol) at 0° C. The mixture was allowed to warm to 60° C., and then stirred for 16 h. The mixture was concentrated, and the residue was diluted with water and adjusted to pH 3 by addition of 1 M HCl_{(aq)}. After extraction of the aq. layer with ethyl acetate, the combined organic layers were dried over sodium sulfate, filtered, and concentrated to give PA.4 (5.5 g) which was used in next step without purification. ¹H NMR (400 MHz, DMSO-d₆), δ: 9.43 (d, J=8.6 Hz, 1H), 4.43 (d, J=8.6 Hz, 1H), 4.14 (s, 1H), 3.84 (dd, J=10.8, 5.6 Hz, 1H), 3.71 (d, J=10.8 Hz, 1H), 1.52 (dd, J=7.6, 5.2 Hz, 1H), 1.42 (d, J=7.6 Hz, 1H), 1.00 (s, 12H), 0.81 (s, 3H).

### Synthesis of (1R,2S,5S)-3-((S)-3,3-dimethyl-2-(methylsulfonamido)butanoyl)-6,6-dimethyl-3-azabicyclo[3.1.0]hexane-2-carboxylic acid (PA.7)

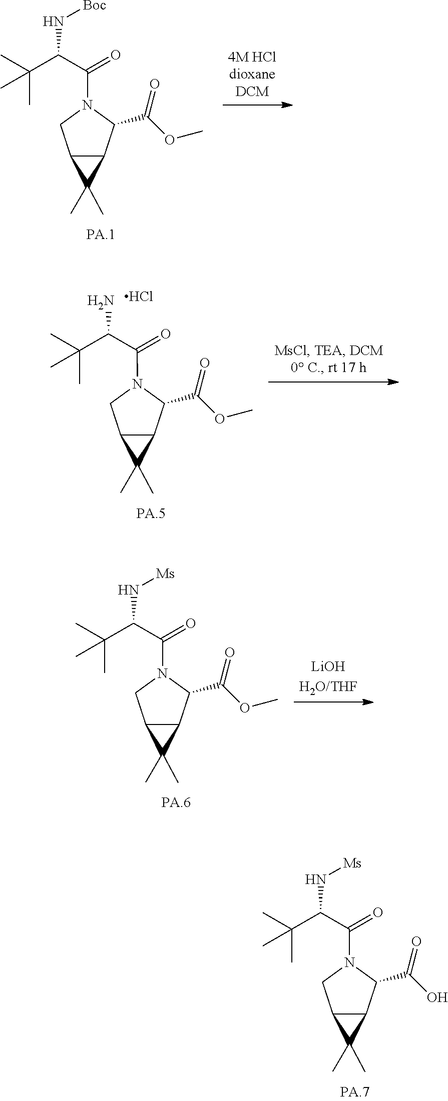 [SMILES: COC(=O)[C@@H]1[C@@H]2[C@H](CN1C(=O)[C@@H](NC(=O)OC(C)(C)C)C(C)(C)C)C2(C)C ; Cl ; COC(=O)[C@@H]1[C@@H]2[C@H](CN1C(=O)[C@@H](N)C(C)(C)C)C2(C)C ; COC(=O)[C@@H]1[C@@H]2[C@H](CN1C(=O)[C@@H](NS(C)(=O)=O)C(C)(C)C)C2(C)C ; CC(C)(C)[C@H](NS(C)(=O)=O)C(=O)N1C[C@H]2[C@@H]([C@H]1C(=O)O)C2(C)C]

### Preparation 5. Methyl (1R,2S,5S)-3-((S)-2-amino-3,3-dimethylbutanoyl)-6,6-dimethyl-3-azabicyclo[3.1.0]hexane-2-carboxylate hydrochloride (PA.5)

A solution of HCl in 1,4-dioxane (4 M, 20 ml) was added to a solution of PA.1 (5.0 g, 13.08 mmol) in DCM (20 ml). After the mixture was stirred at 25° C. for 16 h, the solvent was removed under reduced pressure to give a HCl salt of PA.5 (4.3 g, crude). ¹H NMR (400 MHz, DMSO-d₆), δ: 8.35 (br. s, 3H), 4.22 (s, 1H), 3.83-3.79 (m, 2H), 3.74-3.72 (m, 1H), 3.65 (s, 3H), 1.57-1.56 (m, 1H), 1.46 (s, J=7.6 Hz, 1H), 1.02-1.01 (m, 12H), 0.96 (s, 3H).

### Preparation 6. Methyl (1R,2S,5S)-3-((S)-3,3-dimethyl-2-(methylsulfonamido)butanoyl)-6,6-dimethyl-3-azabicyclo[3.1.0]hexane-2-carboxylate (PA.6)

To a solution of PA.5 (500 mg, 1.58 mmol) in DCM (10 ml) was added MsCl (270 mg, 2.36 mmol) and TEA (640 mg, 6.32 mmol) at 0° C. The reaction mixture was stirred at 25° C. for 17 h. The mixture was concentrated and purified by silica gel column chromatography (n-hexane:ethyl acetate=3:1) to give the desired product PA.6 (480 mg, 84% yield). ¹H NMR (400 MHz, DMSO-d₆), δ: 5.25 (d, J=10.0 Hz, 1H), 4.48 (s, 1H), 3.92-3.88 (m, 1H), 3.85-3.77 (m, 2H), 3.75 (s, 3H), 2.89 (s, 3H), 1.51-1.45 (m, 2H), 1.06-1.05 (m, 12H), 0.97 (s, 3H).

### Preparation 7. (1R,2S,5S)-3-((S)-3,3-Dimethyl-2-(methylsulfonamido)butanoyl)-6,6-dimethyl-3-azabicyclo[3.1.0]hexane-2-carboxylic acid (PA.7)

To a solution of PA.6 (480 mg, 1.33 mmol) in THF (4.0 mL) was added lithium hydroxide (96 mg, 4.0 mmol) and water (4.0 mL). After the reaction mixture was stirred at 25° C. for 17 h, it was neutralized by 1 M HCl_{(aq)} and then concentrated to give PA.7 (800 mg, crude) which was used in next step. ¹H NMR (400 MHz, DMSO-d₆), δ: 7.07 (d, J=9.6 Hz, 1H), 4.12 (s, 1H), 3.82-3.76 (m, 2H), 3.40-3.67 (m, 1H), 2.83 (s, 3H), 1.54-1.51 (m, 1H), 1.40 (d, J=8.4 Hz, 1H), 1.00 (s, 3H), 0.97 (s, 9H), 0.88 (s, 3H).

### Synthesis of 2-amino-3-(6-methyl-2-oxo-1,2-dihydroquinolin-3-yl)propanamide hydrogen chloride (P1.5)

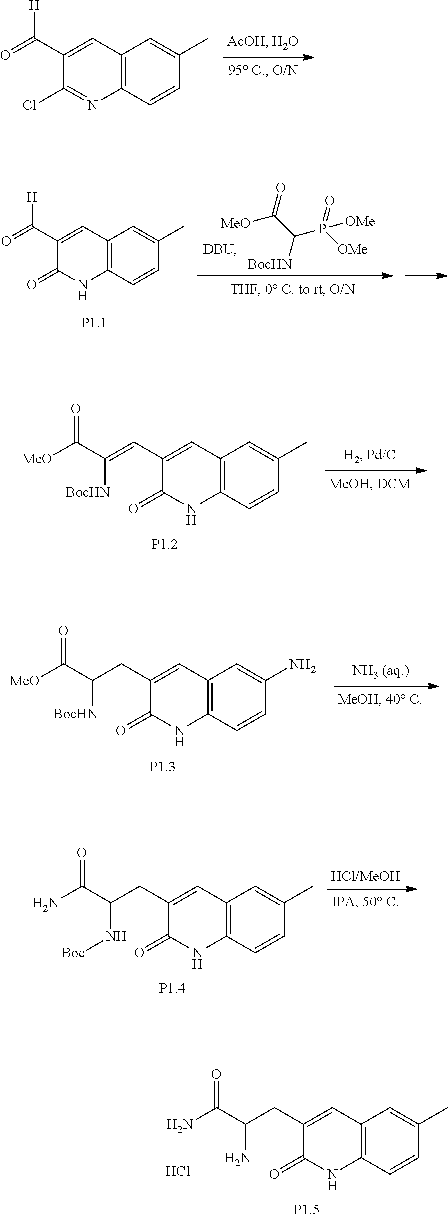

### Preparation 8. 6-methyl-2-oxo-1,2-dihydroquinoline-3-carbaldehyde (P1.1)

A solution of 2-chloro-6-methyl-quinoline-3-carbaldehyde (4.0 g, 19.45 mmol) in 70% AcOH_{(aq)} (385 mL) was stirred at 95° C. for 16 h. The mixture was cooled to rt. The resulting solid was collected by filtration to give P1.1 (2.75 g, 79%) as a yellow solid. ¹H NMR (400 MHz, CDCl₃), δ: 10.47 (s, 1H), 8.42 (s, 1H), 7.51 (s, 1H), 7.47 (d, J=8.0 Hz, 1H), 7.21 (d, J=8.0 Hz, 1H), 2.44 (s, 3H).

### Preparation 9. Methyl (Z)-2-((tert-butoxycarbonyl)amino)-3-(6-methyl-2-oxo-1,2-dihydroquinolin-3-yl)acrylate (P1.2)

To a solution of (±)-Boc-α-phosphonoglycine trimethyl ester (2.1 g, 7.05 mmol) in anh. THF (15 mL) was added 1,8-diazabicyclo[5.4.0]undec-7-ene (1.05 mL, 7.05 mmol) slowly at 0° C. After the reaction was stirred at 0° C. for 30 min, the ylide solution was added slowly into a solution of P1.1 (1.0 g, 5.34 mmol) in anh. THF (10 mL) at 0° C. The mixture was warmed to rt and stirred overnight. The solvent was removed under reduced pressure. The residue was purified by silica gel column chromatography (0-70% EtOAc in n-hexane) to give P1.2 (1.27 g, 66%) as a yellow solid. ¹H NMR (400 MHz, CDCl₃), δ: 12.22 (br. s, 1H), 7.91 (s, 1H), 7.39-7.38 (m, 2H), 7.36 (s, 1H), 6.83 (s, 1H), 3.90 (s, 3H), 2.43 (s, 3H), 1.41 (s, 9H); LCMS (ESI) m/z: calc. for C₁₉H₂₂N₂O₅ 358.15. found, 359.2 [M+H]⁺.

### Preparation 10. Methyl 2-((tert-butoxycarbonyl)amino)-3-(6-methyl-2-oxo-1,2-dihydroquinolin-3-yl)propanoate (P1.3)

To a solution of P1.2 (0.10 g, 0.28 mmol) in methanol (5.0 mL) and DCM (5.0 mL) was added Pd/C (0.01 g, 10% wt). The reaction was stirred overnight under hydrogen atmosphere (1 atm). The solution was filtered through a pad of celite. The filtrate was concentrated under reduced pressure. The residue was purified by silica gel column chromatography (0-100% EtOAc in n-hexane) to give P1.3 (90 mg, 89%) as a solid. ¹H NMR (400 MHz, CDCl₃), δ: 11.40 (br. s, 1H), 7.34-7.31 (m, 2H), 7.27-7.25 (m, 2H), 6.28 (d, J=8.0 Hz, 1H), 4.58-4.53 (m, 1H), 3.72 (s, 3H), 3.19-3.08 (m, 2H), 2.41 (s, 3H), 1.36 (s, 9H); LCMS (ESI) m/z: calc. for C₁₉H₂₄N₂O₅ 360.17. found, 361.0 [M+H]⁺.

### Preparation 11. tert-Butyl (1-amino-3-(6-methyl-2-oxo-1,2-dihydroquinolin-3-yl)-1-oxopropan-2-yl)carbamate (P1.4)

To a solution of P1.3 (0.13 g, 0.36 mmol) in methanol (4 mL) and DCM (1.0 mL), was added ammonium hydroxide (1.0 mL, 30%). The reaction was stirred at 40° C. overnight. The solvent was removed under reduced pressure to give P1.4 (0.12 g, 96%) as a solid which was used without further purification. ¹H NMR (400 MHz, DMSO-d₆), δ: 7.61 (s, 1H), 7.34 (s, 1H), 7.28-7.17 (m, 3H), 6.97 (s, 1H), 6.91 (d, J=8.0 Hz, 1H), 4.17-4.11 (m, 1H), 2.91-2.89 (m, 1H), 2.66-2.61 (m, 1H), 2.32 (s, 3H), 1.24 (s, 9H); LCMS (ESI) m/z: calc. for C₁₈H₂₃N₃O₄ 345.17. found, 346.1 [M+H]⁺.

### Preparation 12. 2-Amino-3-(6-methyl-2-oxo-1,2-dihydroquinolin-3-yl)propanamide hydrogen chloride (P1.5)

To a solution of P1.4 (0.34 g, 1.0 mmol) in IPA (7.0 mL) HCl (1.67 mL, 3M in MeOH) was added in ice bath. The reaction was stirred at 50° C. for overnight. The solvent was removed under reduced pressure. The resulted solid was filtered and washed by IPA to give P1.5 (0.149 g, 53%) as a solid. ¹H NMR (400 MHz, DMSO-d₆), δ: 11.92 (s, 1H), 8.18 (br. s, 3H), 7.87 (br. s, 1H), 7.72 (d, J=4.0 Hz, 1H), 7.50 (s, 1H), 7.38 (s, 1H), 7.31 (d, J=8.4 Hz, 1H), 7.22 (d, J=8.4 Hz, 1H), 4.04-3.99 (m, 1H), 3.01-2.86 (m, 2H), 2.32 (s, 3H); LCMS (ESI) m/z: calc. for C₁₃H₁₅N₃O₂ 245.12. found, 246.1 [M+H]⁺.

### Synthesis of 2-amino-3-(7-fluoro-2-oxo-1,2-dihydroquinolin-3-yl)propanamide hydrochloride (P1.12)

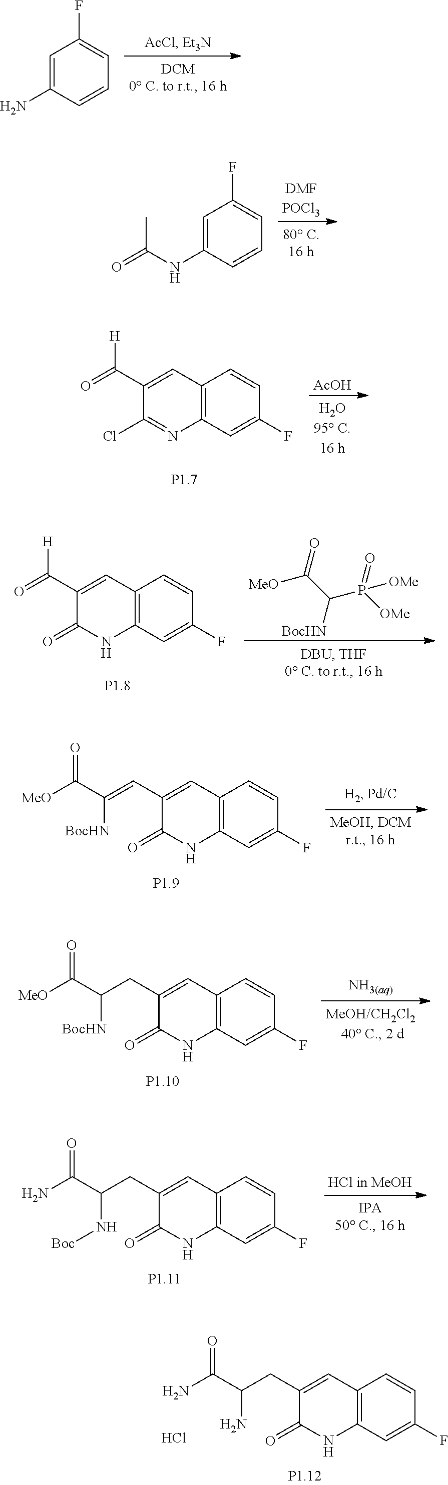

Preparation 13. N-(3-Fluorophenyl)acetamide (P1.6). To a solution of 3-fluoroaniline (5.00 g, 45.0 mmol, 1.0 eq) in DCM (230 mL) was added triethylamine (18.8 mL, 135 mmol, 3.0 eq) and acetyl chloride (4.80 mL, 67.5 mmol, 1.5 eq) dropwise at 0° C., and then stirred at rt for 16 h. The mixture was treated with water and extracted with DCM three times. The combined organic layers were dried over Na₂SO₄₍ₛ₎, filtered, and concentrated in vacuo. The residue was purified by silica gel column chromatography (n-hexane:EtOAc=2:1) to give P1.6 (7.0 g, 100% yield) as a yellow solid. ¹H NMR (400 MHz, CDCl₃), δ: 7.50-7.48 (m, 1H), 7.36 (br. s, 1H), 7.28-7.21 (m, 1H), 7.12 (d, J=8.4 Hz, 1H), 6.82-6.78 (m, 1H), 2.18 (s, 3H).

### Preparation 14. 2-Chloro-7-fluoroquinoline-3-carbaldehyde (P1.7)

DMF (3.8 mL, 40 mmol, 2.5 eq) was cooled to 0° C. and phosphoryl chloride (12.8 mL, 137 mmol, 7.0 eq) was added dropwise with stirring. To the reaction mixture was added P1.6 (3.0 g, 19.6 mmol, 1.0 eq). After the reaction was stirred at 80° C. for 16 h, the reaction mixture was cooled to rt and then poured into ice water. The precipitate was collected by filtration and washed with water to give P1.7 (2.76 g, 67% yield) as a light yellow solid. ¹H NMR (400 MHz, CDCl₃); δ: 10.55 (s, 1H), 8.76 (s, 1H), 8.03-7.99 (m, 1H), 7.73-7.69 (m, 1H), 7.47-7.42 (m, 1H).

### Preparation 15. 7-Fluoro-2-oxo-1,2-dihydroquinoline-3-carbaldehyde (P1.8)

A solution of P1.7 (2.76 g, 13.2 mmol, 1.0 eq) in 70% AcOH_{(aq)} (110 mL) was stirred at 95° C. for 16 h and then cooled to rt. The resulting precipitate was collected by filtration and washed with water to give P1.8 (1.87 g, 74% yield) as a yellow solid. ¹H NMR (400 MHz, DMSO-d₆), δ: 12.32 (br. s, 1H), 10.20 (s, 1H), 8.53 (s, 1H), 8.03 (dd, J=8.8, 6.0 Hz, 1H), 7.19-7.13 (m, 1H), 7.08 (dd, J=10.4, 2.8 Hz, 1H).

### Preparation 16. Methyl (Z)-2-((tert-butoxycarbonyl)amino)-3-(7-fluoro-2-oxo-1,2-dihydroquinolin-3-yl)acrylate (P1.9)

To a solution of (±)-Boc-α-phosphonoglycine trimethyl ester (2.05 g, 6.91 mmol, 1.32 eq) in anh. THF (17 mL) was added 1,8-diazabicyclo[5.4.0]undec-7-ene (DBU, 1.03 mL, 6.91 mmol, 1.32 eq) dropwise at 0° C. After the reaction was stirred at 0° C. for 30 min, the mixture was added slowly into a solution of P1.8 (1.00 g, 5.23 mmol, 1.0 eq) in anh. THF (26 mL) at 0° C. The mixture was warmed to rt and stirred for 16 h. The reaction mixture was concentrated and purified by silica gel column chromatography (n-hexane:EtOAc=3:1 to 2:1) to give P1.9 (1.58 g, 83% yield) as a yellow solid. ¹H NMR (400 MHz, CDCl₃), δ: 12.08 (br. s, 1H), 8.40 (s, 1H), 7.95 (s, 1H), 7.59-7.55 (m, 1H), 7.17-7.11 (m, 1H), 7.03-7.00 (m, 1H), 6.94 (s, 1H), 3.90 (s, 3H), 1.41 (s, 9H).

### Preparation 17. Methyl 2-((tert-butoxycarbonyl)amino)-3-(7-fluoro-2-oxo-1,2-dihydroquinolin-3-yl)propanoate (P1.10)

To a solution of P1.9 (1.58 g, 4.36 mmol, 1.0 eq) in methanol (23 mL) and DCM (23 mL) was added Pd/C powder (160 mg, 10% wt). The reaction was stirred for 16 h under hydrogen atmosphere (1 atm). The solution was filtered through a pad of celite. The filtrate was concentrated to give crude P1.10 (1.57 g, 99% yield) as a white solid. ¹H NMR (400 MHz, CDCl₃), δ: 11.37 (s, 1H), 7.65 (s, 1H), 7.52-7.49 (m, 1H), 7.06-7.03 (m, 1H), 6.98-6.93 (m, 1H), 5.98 (d, J=8.0 Hz, 1H), 4.63-4.56 (m, 1H), 3.74 (s, 3H), 3.21-3.17 (m, 1H), 3.07-3.02 (m, 1H), 1.35 (s, 9H).

### Preparation 18. tert-Butyl (1-amino-3-(7-fluoro-2-oxo-1,2-dihydroquinolin-3-yl)-1-oxopropan-2-yl)carbamate (P1.11)

To a solution of P1.10 (300 mg, 0.820 mmol, 1.0 eq) in methanol (5.0 mL) and DCM (5.0 mL) was added ammonium hydroxide (2.0 mL, 30%). The reaction was stirred at 40° C. for 2 days. The precipitate was collected by filtration, washed with methanol and IPA, and then dried under air to give P1.11 (145 mg, 50% yield) as a white solid. ¹H NMR (400 MHz, DMSO-d₆), δ: 11.87 (br. s, 1H), 7.69 (s, 1H), 7.65-7.62 (m, 1H), 7.25 (s, 1H), 7.06-6.98 (m, 3H), 6.86 (d, J=8.4 Hz, 1H), 4.22-4.13 (m, 1H), 2.96-2.92 (m, 1H), 2.62-2.56 (m, 1H), 1.22 (s, 9H).

### Preparation 19. 2-Amino-3-(7-fluoro-2-oxo-1,2-dihydroquinolin-3-yl)propanamide hydrochloride (P1.12)

To a solution of P1.11 (145 mg, 0.42 mmol, 1.0 eq) in IPA (2.9 mL) was added a solution of HCl in methanol (0.690 mL, 2.08 mmol, 5.0 eq, 3 M). The reaction was stirred at 50° C. for 16 h. The precipitate was collected by filtration, washed with IPA, and dried under air to give P1.12 (92 mg, 89% yield) as a white solid. ¹H NMR (400 MHz, DMSO-d₆), δ: 12.09 (br. s, 1H), 8.21 (br. s, 3H), 7.93 (s, 1H), 7.83 (s, 1H), 7.71-7.69 (m, 1H), 7.55 (s, 1H), 7.12-7.04 (m, 2H), 4.07-4.01 (m, 1H), 3.04-2.88 (m, 2H).

### Synthesis of 2-amino-3-(6-methoxy-2-oxo-1,2-dihydroquinolin-3-yl)propanamide (P1.19)

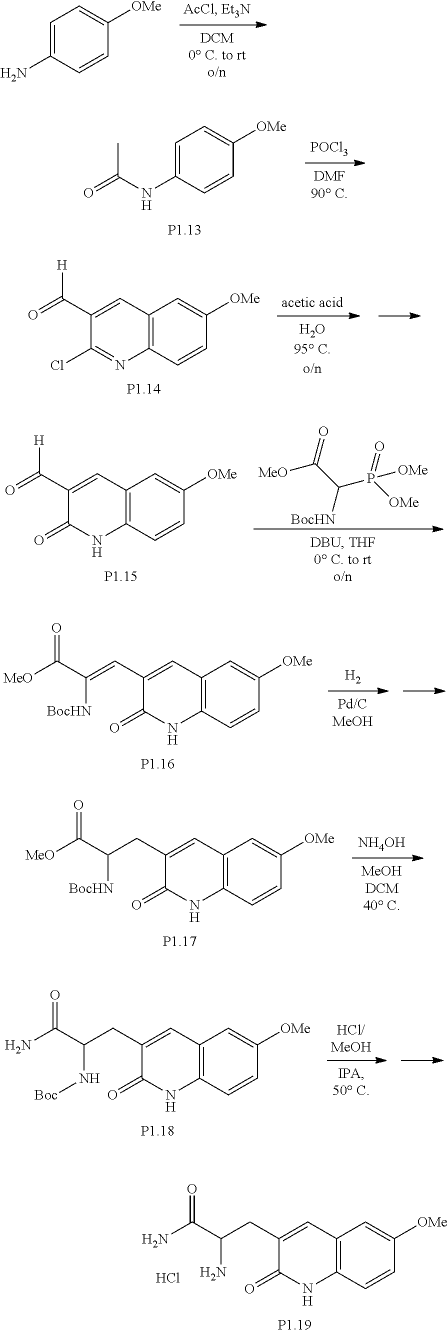

### Preparation 20. N-(4-Methoxyphenyl)acetamide (P1.13)

To a solution of 4-methoxyaniline (5.0 g, 41 mmol) in anh. DCM (81 mL) was added triethylamine (8.5 mL, 61 mmol) and then stirred at 0° C. for 15 min. The mixture was added by AcCl at 0° C. slowly. The mixture stirred at rt for 16 h. The mixture was treated with water and extracted with EtOAc. The combined organic layers were dried over Na₂SO₄, filtered, and concentrated. The residue was purified by silica gel column chromatography (0-100% ethyl ethanoate in n-hexane) to give P1.13 (7.2 g, 99%) as a black solid. ¹H NMR (400 MHz, CDCl₃), δ: 7.38 (d, J=9.0 Hz, 2H), 7.27 (br. s, 1H), 6.84 (d, J=9.0 Hz, 2H), 3.78 (s, 3H), 2.14 (s, 3H).

### Preparation 21. 2-Chloro-6-methoxyquinoline-3-carbaldehyde (P1.14)

A solution of phosphoryl chloride (63 mL, 680 mmol) in anhydrous N,N-Dimethylformamide (15 mL) was stirred at 0° C. for 15 min and then stirred at rt for 15 min. After P1.13 (12.48 g, 75.55 mmol) was added to the reaction at 0° C., the mixture was stirred at 90° C. for 16 h. The mixture was treated with water and extracted with EtOAc. The combined organic layers were dried over Na₂SO₄₍ₛ₎, filtered, and concentrated under reduced pressure to give P1.14 (11.2 g, 66%) as a brown solid which was used without further purification. ¹H NMR (400 MHz, CDCl₃), δ: 10.55 (s, 1H), 8.64 (s, 1H), 7.97 (d, J=9.2 Hz, 1H), 7.52 (dd, J=9.2, 2.8 Hz, 1H), 7.20 (d, J=2.8 Hz, 1H), 3.95 (s, 3H).

### Preparation 22. 6-Methoxy-2-oxo-1,2-dihydroquinoline-3-carbaldehyde (P1.15)

A solution of P1.14 (11.2 g, 50.53 mmol) in 75% AcOH_{(aq)} (505 mL) was stirred at 95° C. for 16 h. The mixture was cooled to room temperature and treated with ice water. The resulting solid was collected by filtration to give P1.15 (9.0 g, 87%) as a yellow solid. ¹H NMR (400 MHz, CDCl₃), δ: 10.49 (s, 1H), 8.42 (s, 1H), 7.34-7.29 (m, 2H), 7.11-7.08 (m, 1H), 3.87 (s, 3H).

### Preparation 23. Methyl (Z)-2-((tert-butoxycarbonyl)amino)-3-(6-methoxy-2-oxo-1,2-dihydroquinolin-3-yl)acrylate (P1.16)

To a solution of (±)-Boc-α-phosphonoglycine trimethyl ester (3.80 g, 13.0 mmol) in anh. THF (58 mL) was added 1,8-diazabicyclo[5.4.0]undec-7-ene (DBU, 1.97 mL, 13.0 mmol) slowly at 0° C. After the reaction was stirred at 0° C. for 30 min, the ylide was added slowly into a solution of P1.15 (2.0 g, 9.84 mmol) in anh. THF (40 mL) at 0° C. The mixture was stirred at rt for 16 h. The solvent was removed under reduced pressure. The residue was purified by silica gel column chromatography (0-100% EtOAc in n-hexane) to give P1.16 (1.1 g, 15%) as a yellow solid. ¹H NMR (400 MHz, CDCl₃), δ: 8.92 (br. s, 1H), 7.90 (s, 1H), 7.37 (d, J=9.0 Hz, 1H), 7.22 (dd, J=9.0, 2.8 Hz, 1H), 6.97 (d, J=2.8 Hz, 1H), 6.82 (s, 1H), 3.89 (s, 3H), 3.87 (s, 3H), 1.41 (s, 9H).

### Preparation 24. Methyl 2-((tert-butoxycarbonyl)amino)-3-(6-methoxy-2-oxo-1,2-dihydroquinolin-3-yl)propanoate (P1.17)

To a solution of P1.16 (1.10 g, 2.94 mmol) in methanol (49 mL) was added Pd/C (312 mg, 10% wt). After the reaction was stirred overnight under hydrogen balloon (1 atm), the solution was filtered through a pad of celite and washed with solvent. The filtrate was concentrated under reduced pressure to give P1.17 (800 mg, 72%) as a yellow solid which was used without further purification. ¹H NMR (400 MHz, CDCl₃), δ: 11.77 (br. s, 1H), 7.65 (s, 1H), 7.33 (d, J=9.0 Hz, 1H), 7.15 (dd, J=9.0, 2.8 Hz, 1H), 6.95 (d, J=2.8 Hz, 1H), 6.30 (d, J=6.4 Hz, 1H), 4.59-4.54 (m, 1H), 3.86 (s, 3H), 3.72 (s, 3H), 3.19-3.08 (m, 2H), 1.36 (s, 9H).

### Preparation 25. tert-Butyl (1-amino-3-(6-methoxy-2-oxo-1,2-dihydroquinolin-3-yl)-1-oxopropan-2-yl)carbamate (P1.18)

To a solution of P1.17 (866 mg, 2.30 mmol) in methanol (40 mL) and DCM (5.0 mL), was added ammonium hydroxide (10 mL, 30%). The reaction was stirred at 40° C. for 5 days. The solvent was removed under reduced pressure to give P1.18 (960 mg, <99%) as a yellow solid which was used in next step without further purification. ¹H NMR (400 MHz, DMSO-d₆), δ: 7.63 (s, 1H), 7.22-7.18 (m, 2H), 7.11-7.06 (m, 2H), 6.98-6.92 (m, 1H), 4.18-4.12 (m, 1H), 3.76 (s, 3H), 2.94-2.89 (m, 1H), 2.67-2.61 (m, 1H), 1.24 (s, 9H).

### Preparation 26. 2-Amino-3-(6-methoxy-2-oxo-1,2-dihydroquinolin-3-yl)propanamide (P1.19)

To a solution of P1.19 (660 mg, 1.58 mmol) in IPA (11 mL) was added HCl (2.6 mL, 3M in MeOH) in ice bath. The reaction was stirred at 50° C. for 16 h. The solvent was removed under reduced pressure. The resulted solid was collected and washed by IPA to give P1.19 (292 g, 70%) as a white solid. ¹H NMR (400 MHz, DMSO-d₆), δ: 11.94 (br. s, 1H), 8.21 (br. s, 2H), 7.89 (s, 1H), 7.76 (s, 1H), 7.53 (s, 1H), 7.29-7.27 (m, 1H), 7.16-7.14 (m, 2H), 4.06-4.03 (m, 1H), 3.77 (s, 3H), 3.04-2.90 (m, 2H).

### Synthesis of (1R,2S,5S)—N-(1-amino-3-(6-methyl-2-oxo-1,2-dihydroquinolin-3-yl)-1-oxopropan-2-yl)-3-((S)-3,3-dimethyl-2-(methylsulfonamido)butanoyl)-6,6-dimethyl-3-azabicyclo[3.1.0]hexane-2-carboxamide (P1.20)

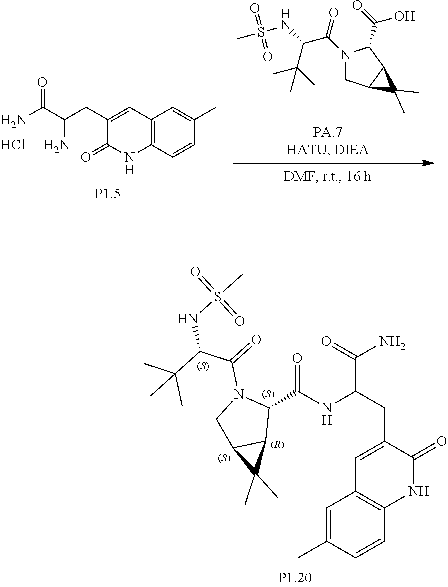

### Preparation 27. (1R,2S,5S)—N-(1-Amino-3-(6-methyl-2-oxo-1,2-dihydroquinolin-3-yl)-1-oxopropan-2-yl)-3-((S)-3,3-dimethyl-2-(methylsulfonamido)butanoyl)-6,6-dimethyl-3-azabicyclo[3.1.0]hexane-2-carboxamide (P1.20)

To a solution of PA.7 (300.0 mg, 0.779 mmol, 1.0 eq, 90%) in DMF (5.5 mL) was added P1.5 (263.5 mg, 0.935 mmol, 1.2 eq), 1-[bis(dimethylamino)methylene]-1H-1,2,3-triazolo[4,5-b]pyridinium 3-oxide hexafluorophosphate (HATU, 326.0 mg, 0.857 mmol, 1.1 eq) and N,N-Diisopropylethylamine (DIEA, 0.41 mL, 2.34 mmol, 3.0 eq). The reaction mixture was stirred at room temperature for 16 hours. The mixture was concentrated and purified by C-18 reversed-phase column chromatography (mobile phase A: water (with 1% NH₄HCO₃), mobile phase B: methanol, UV: 214 and 254 nm, Flow rate: 60 mL/min, Gradient: 3-90% (% B)) to give P1.20 (308 mg, 69% yield) as a white solid. ¹H NMR (400 MHz, DMSO-d₆), δ 11.76-11.72 (m, 1H), 8.62-8.34 (m, 1H), 7.63-7.61 (m, 1H), 7.39-6.90 (m, 6H), 4.53-4.31 (m, 1H), 4.13 (s, 1H), 3.90-3.57 (m, 3H), 3.25-3.15 (m, 1H), 2.98-2.86 (m, 1H), 2.83 (m, 3H), 2.31-2.30 (m, 3H), 1.54-1.29 (m, 1H), 1.18 (d, J=7.6 Hz, 0.5H), 0.98-0.79 (m, 15H), 0.76 (d, J=8.0 Hz, 0.5H).

### Synthesis of (1R,2S,5S)—N-(1-amino-3-(7-methyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.28)

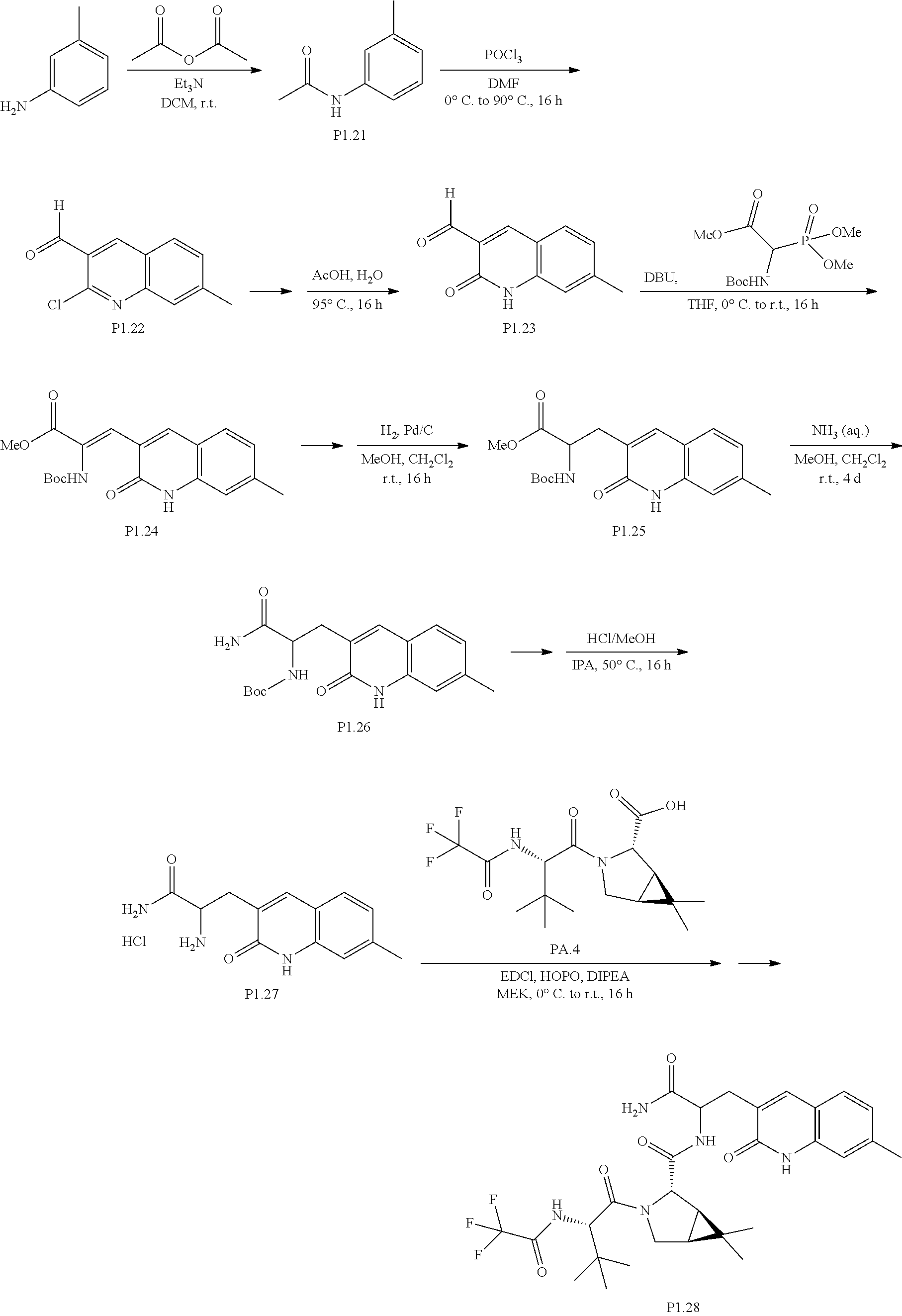

### Preparation 28. N-(m-Tolyl)acetamide (P1.21)

A solution of 3-methylaniline (10.0 g, 93.3 mmol, 1.0 eq) and TEA (20 mL, 139.95 mmol, 1.5 eq) in DCM (155 mL) was stirred at 0° C. for 15 min. To the mixture was added acetic anhydride (9.7 mL, 102.6 mmol, 1.1 eq) at 0° C. slowly and it was stirred at rt for 16 h. The mixture was treated with water and extracted with EtOAc. The combined organic layers were dried over Na₂SO₄₍ₛ₎, filtered, and concentrated to give P1.21 (12.5 g, 89% yield) as a brown solid which was used in next step without further purification. ¹H NMR (400 MHz, CDCl₃), δ: 7.74 (br. s, 1H), 7.35 (s, 1H), 7.30-7.26 (m, 1H), 7.17 (dd, J=8.0, 7.6 Hz, 1H), 6.90 (d, J=7.6 Hz, 1H), 2.30 (s, 3H), 2.14 (s, 3H); LCMS (ESI) m/z calc. for C₉H₁₁NO 149.08. found, 149.9 [M+H]⁺.

### Preparation 29. 2-Chloro-7-methylquinoline-3-carbaldehyde (P1.22)

A solution of phosphoryl chloride (70 mL, 754.1 mmol, 9 eq) in anh. DMF (17 mL) was stirred at 0° C. for 10 min and at rt for 15 min. To the mixture was added P1.21 (12.5 g, 83.8 mmol, 1.0 eq) at 0° C. and the solution was stirred at 90° C. for 16 h. The mixture was cooled, poured into ice water, and extracted with EtOAc. The combined organic layers were dried over Na₂SO₄, filtered, and concentrated to give P1.22 (11 g, 63% yield) as a yellow solid which was used in next step without further purification. ¹H NMR (400 MHz, CDCl₃), δ: 10.53 (s, 1H), 8.70 (s, 1H), 7.87-7.84 (m, 2H), 7.49-7.84 (m, 1H), 2.60 (s, 3H); LCMS (ESI) m/z calc. for C₁₁H₈ClNO 205.03. found, 206.1 [M+H]⁺.

### Preparation 30. 7-Methyl-2-oxo-1,2-dihydroquinoline-3-carbaldehyde (P1.23)

A solution of P1.22 (11.0 g, 53.49 mmol, 1.0 eq) in 70% AcOH_{(aq)} (535 mL) was stirred at 95° C. for 16 h. The mixture was cooled to rt. The resulting precipitate was collected by filtration to give P1.23 (6.6 g, 65% yield) as a yellow solid. ¹H NMR (400 MHz, CDCl₃), δ: 11.24 (br. s, 1H), 10.48 (s, 1H), 8.45 (s, 1H), 7.62 (d, J=8.4 Hz, 1H), 7.16 (s, 1H), 7.12 (d, J=8.4 Hz, 1H), 2.51 (s, 3H); LCMS (ESI) m/z calc. for C₁₁H₉NO₂ 187.06. found, 188.0 [M+H]⁺.

### Preparation 31. Methyl (Z)-2-((tert-butoxycarbonyl)amino)-3-(7-methyl-2-oxo-1,2-dihydroquinolin-3-yl)acrylate (P1.24)

To a solution of (±)-Boc-α-phosphonoglycine trimethyl ester (2.1 g, 7.1 mmol, 1.32 eq) in anh. THF (26.5 mL) was added 1,8-diazabicyclo[5.4.0]undec-7-ene (DBU, 1.1 mL, 7.1 mmol, 1.32 eq) slowly at 0° C. After the reaction was stirred at 0° C. for 30 min, the ylide solution was added slowly into a solution of P1.23 (1.0 g, 5.3 mmol, 1.0 eq) in anh. THF (26.5 mL) at 0° C. The mixture was stirred at rt for 16 h. The solvent was removed under reduced pressure. The residue was purified by silica gel column chromatography (0-100% EtOAc in n-hexane) to give P1.24 (750 mg, 39% yield) as a yellow solid. ¹H NMR (400 MHz, CDCl₃), δ: 8.60 (br. s, 1H), 7.92 (s, 1H), 7.47 (d, J=8.2 Hz, 1H), 7.16 (s, 1H), 7.10-7.07 (m, 1H), 6.89 (s, 1H), 3.88 (s, 3H), 2.50 (s, 3H), 1.41 (s, 9H); LCMS (ESI) m/z calc. for C₁₉H₂₂N₂O₅ 358.15. found, 359.2 [M+H]⁺.

### Preparation 32. Methyl 2-((tert-butoxycarbonyl)amino)-3-(7-methyl-2-oxo-1,2-dihydroquinolin-3-yl)propanoate (P1.25)

To a solution of P1.24 (750 mg, 2.1 mmol, 1.0 eq) in methanol (35 mL) was added Pd/C powder (222 mg). The reaction was stirred at rt for 16 h under hydrogen atmosphere. The solution was filtered through a pad of celite and washed the pad thoroughly. The filtrate was concentrated under reduced pressure to give P1.25 (735 mg, 97% yield) as a yellow solid. ¹H NMR (400 MHz, CDCl₃), δ: 10.98 (br. s, 1H), 7.64 (s, 1H), 7.41 (d, J=8.0 Hz, 1H), 7.13 (s, 1H), 7.04 (d, J=8.0 Hz, 1H), 6.07 (br. s, 1H), 4.56 (br. s, 1H), 3.72 (s, 3H), 3.17-3.05 (m, 2H), 2.48 (s, 3H), 1.35 (s, 9H); LCMS (ESI) m/z calc. for C₁₉H₂₄N₂O₅ 360.17. found, 361.2 [M+H]⁺.

### Preparation 33. tert-Butyl (1-amino-3-(7-methyl-2-oxo-1,2-dihydroquinolin-3-yl)-1-oxopropan-2-yl)carbamate (P1.26)

To a solution of P1.25 (735 mg, 2.0 mmol, 1.0 eq.) in methanol (35 mL) and DCM (5.0 mL) was added 30% ammonium hydroxide (8.8 mL). The reaction was stirred at rt for 4 days. The solvent was removed under reduced pressure. The residue was treated and washed with DCM. The resulting precipitate was collected by filtration to give P1.26 (437 mg, 62% yield) as a yellow solid which was used in next step without further purification. ¹H NMR (400 MHz, DMSO-d₆), δ: 7.63 (s, 1H), 7.44 (d, J=8.0 Hz, 1H), 7.22 (s, 1H), 7.07 (s, 1H), 7.04-6.94 (m, 2H), 6.90 (d, J=8.4 Hz, 1H), 4.17-4.11 (m, 1H), 2.93-2.88 (m, 1H), 2.69-2.56 (m, 1H), 2.35 (s, 3H), 1.24 (s, 9H); LCMS (ESI) m/z calc. for C₁₈H₂₃N₃O₄ 345.17. found, 346.2 [M+H]⁺.

### Preparation 34. 2-Amino-3-(7-methyl-2-oxo-1,2-dihydroquinolin-3-yl)propanamide hydrochloride (P1.27)

To a solution of P1.26 (437 mg, 1.3 mmol, 1.0 eq.) in IPA (9.0 mL) was added a solution of 3M HCl in MeOH (2.2 mL, 6.4 mmol, 5.0 eq.). The reaction was stirred at 50° C. for 16 h and cooled. The resulting precipitate was collected by filtration, washed with methanol, and dried to give P1.27 (129 mg, 36% yield) as a yellow solid. ¹H NMR (400 MHz, DMSO-d₆), δ: 11.94 (br. s, 1H), 8.26 (s, 3H), 7.94 (s, 1H), 7.77 (s, 1H), 7.52-7.50 (m, 2H), 7.13 (s, 1H), 7.03-7.01 (m, 1H), 4.05-4.02 (m, 1H), 3.06-2.87 (m, 2H), 2.37 (s, 3H); LCMS (ESI) m/z calc. for C₁₃H₁₅N₃O₂ 245.12. found, 246.1 [M+H]⁺.

### Preparation 35. (1R,2S,5S)—N-(1-Amino-3-(7-methyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.28)

To a solution of P1.27 (129 mg, 0.5 mmol, 1.1 eq.), PA.4 (151 mg, 0.41 mmol, 1.0 eq.) in butan-2-one (MEK, 1.5 mL) was added 2-hydroxypyridine 1-oxide (HOPO, 12 mg, 0.1 mmol, 0.25 eq.) and 1-(3-dimethylaminopropyl)-3-ethylcarbodiimide hydrochloride (EDCI, 104 mg, 0.54 mmol, 1.3 eq.) at 0° C. After N,N-diisopropylethylamine (DIPEA, 290 μL, 1.7 mmol, 4.0 eq.) was added slowly to the reaction mixture, the resulting mixture was stirred at room temperature for 16 h. The mixture was concentrated and then purified by C-18 reversed-phase column chromatography (0-100% MeOH in water containing 1% NH₄HCO₃) to give P1.28 (210 mg, 77% yield) as a yellow solid. ¹H NMR (400 MHz, DMSO-d₆), δ: 11.81-11.72 (m, 2H), 9.47-9.35 (m, 2H), 8.55-8.31 (m, 2H), 7.67-7.61 (m, 2H), 7.49-7.40 (m, 2H), 7.07-6.95 (m, 8H), 4.47-4.32 (m, 4H), 4.13-4.08 (m, 2H), 4.00-3.78 (m, 2H), 3.60-3.58 (m, 2H), 3.15-3.10 (m, 2H), 2.93-2.79 (m, 2H), 2.60-2.54 (m, 2H), 2.34-2.33 (m, 6H), 1.48-1.29 (m, 2H), 1.09 (d, J=7.8 Hz, 1H), 0.96-0.78 (m, 31H); LCMS (ESI) m/z calc. for C₂₉H₃₆F₃N₅O₅ 591.27. found, 592.8 [M+H]⁺.

### Synthesis of (1R,2S,5S)—N-(1-amino-3-(6,7-dimethyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.35)

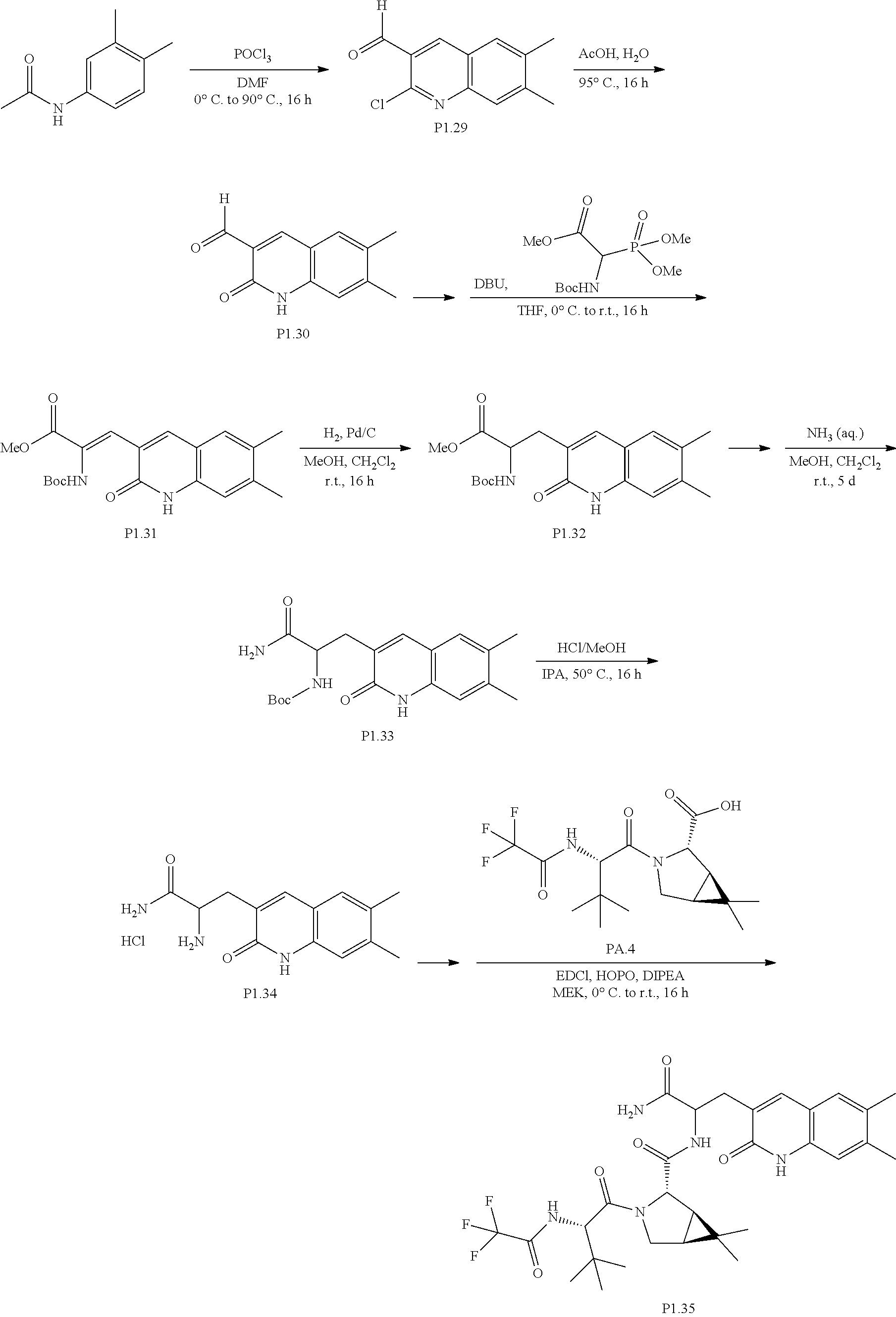

Preparation 36. 2-Chloro-6,7-dimethylquinoline-3-carbaldehyde (P1.29). A solution of phosphoryl chloride (52.0 mL, 551 mmol, 9 eq.) in anh. DMF (13 mL) was stirred at 0° C. for 10 min, then warmed to rt and stirred for 15 min. To the mixture was added N-(3,4-dimethylphenyl)acetamide (10 g, 61.27 mmol, 1.0 eq.) at 0° C. and then stirred at 90° C. for 16 h. The mixture was cooled, treated with ice water, and extracted with EtOAc. The combined organic layers were dried over Na₂SO₄, filtered, and concentrated to give P1.29 (11.1 g, 82% yield) as a yellow solid which was used in next step without further purification. ¹H NMR (400 MHz, CDCl₃), δ: 10.51 (s, 1H), 8.64 (s, 1H), 7.84 (s, 1H), 7.69 (s, 1H), 2.50 (s, 3H), 2.46 (s, 3H); LCMS (ESI) m/z calc. for C₁₂H₁₀ClNO 219.05. found, 220.1 [M+H]⁺.

### Preparation 37. 6,7-Dimethyl-2-oxo-1,2-dihydroquinoline-3-carbaldehyde (P1.30)

A solution of P1.29 (11.1 g, 50.5 mmol, 1.0 eq) in 70% AcOH_{(aq)} (505 mL) was stirred at 95° C. for 16 h. After the mixture was cooled to rt, the resulting precipitate was collected by filtration to give P1.30 (9.9 g, 97% yield) as a yellow solid. ¹H NMR (400 MHz, CDCl₃), δ: 11.77 (br. s, 1H), 10.49 (s, 1H), 8.41 (s, 1H), 7.45 (s, 1H), 7.19 (s, 1H), 2.41 (s, 3H), 2.33 (s, 3H); LCMS (ESI) m/z calc. for C₁₂H₁₁NO₂ 201.08. found, 202.1 [M+H]⁺.

### Preparation 38. Methyl (Z)-2-((tert-butoxycarbonyl)amino)-3-(6,7-dimethyl-2-oxo-1,2-dihydroquinolin-3-yl)acrylate (P1.31)

To a solution of (±)-boc-α-phosphonoglycine trimethyl ester (5.90 g, 19.7 mmol, 1.32 eq) in anh. THF (75 mL) was added 1,8-diazabicyclo[5.4.0]undec-7-ene (DBU, 3.0 mL, 20 mmol, 1.32 eq) slowly at 0° C. After the reaction was stirred at 0° C. for 30 min, the ylide solution was added slowly into a solution of P1.30 (3.0 g, 14.91 mmol, 1.0 eq) in anh. THF (75 mL) at 0° C. The mixture was stirred at rt for 16 h. The solvent was removed under reduced pressure. The residue was treated with methanol. The resulting precipitate was collected by filtration to give P1.31 (3.18 g, 57% yield) as a yellow solid. ¹H NMR (400 MHz, CDCl₃), δ: 8.75 (br. s, 1H), 7.87 (s, 1H), 7.31 (s, 1H), 7.13 (s, 1H), 6.83 (s, 1H), 3.87 (s, 3H), 2.40 (s, 3H), 2.32 (s, 3H), 1.41 (s, 9H); LCMS (ESI) m/z calc. for C₂₀H₂₄N₂O₅ 372.17. found, 373.2 [M+H]⁺.

### Preparation 39. Methyl 2-((tert-butoxycarbonyl)amino)-3-(6,7-dimethyl-2-oxo-1,2-dihydroquinolin-3-yl)propanoate (P1.32)

To a solution of P1.31 (3.18 g, 8.54 mmol, 1.0 eq) in methanol (50 mL) and DCM (150 mL) was added Pd/C (906 mg, 0.1 eq). The reaction was stirred at rt for 16 h under hydrogen (1 atm). The solution was filtered through a pad of celite. The filtrate was concentrated under reduced pressure to give P1.32 (3.0 g, 93% yield) as a yellow solid which was used in next step without further purification. ¹H NMR (400 MHz, MeOD-d₄), δ: 7.70 (s, 1H), 7.35 (s, 1H), 7.12 (s, 1H), 4.52-4.48 (m, 1H), 3.68 (s, 3H), 3.21-3.12 (m, 1H), 2.95-2.80 (m, 1H), 2.36 (s, 3H), 2.32 (s, 3H), 1.32 (s, 9H); LCMS (ESI) m/z calc. for C₂₀H₂₆N₂O₅ 374.18. found, 375.2 [M+H]⁺.

### Preparation 40. tert-Butyl (1-amino-3-(6,7-dimethyl-2-oxo-1,2-dihydroquinolin-3-yl)-1-oxopropan-2-yl)carbamate (P1.33)

To a solution of P1.32 (3.0 g, 8.0 mmol) in methanol (60 mL) and DCM (140 mL) was added 30% ammonium hydroxide solution (35 mL). The reaction was stirred at rt for 5 days. The solvent was removed under reduced pressure. The residue was treated with DCM. The resulting precipitate was collected by filtration and washed with dichloromethane to give P1.33 (2.22 g, 77% yield) as a yellow solid which was used in next step without further purification. ¹H NMR (400 MHz, DMSO-d₆), δ: 11.67 (br., 1H), 7.58 (s, 1H), 7.29 (s, 1H), 7.21 (br. s, 1H), 7.04 (s, 1H), 6.96 (br. s, 1H), 6.92 (d, J=8.0 Hz, 1H), 4.16-4.07 (m, 1H), 2.91-2.86 (m, 1H), 2.66-2.60 (m, 1H), 2.26 (s, 3H), 2.23 (s, 3H), 1.25 (s, 9H); LCMS (ESI) m/z calc. for C₁₉H₂₅N₃O₄ 359.18. found, 360.2 [M+H]⁺.

### Preparation 41. 2-Amino-3-(6,7-dimethyl-2-oxo-1,2-dihydroquinolin-3-yl)propanamide hydrochloride (P1.34)

To a solution of P1.33 (2.22 g, 6.18 mmol, 1.0 eq) in IPA (41 mL) was added 3M HCl in MeOH (10.3 mL, 30.9 mmol, 5.0 eq). The reaction was stirred at 50° C. for 16 h. The resulting precipitate was collected by filtration, washed with methanol, and dried to give P1.34 (1.5 g, 82% yield) as a yellow solid. ¹H NMR (400 MHz, DMSO-d₆), δ: 11.88 (br. s, 1H), 8.25 (br. s, 3H), 7.92 (br. s, 1H), 7.71 (s, 1H), 7.51 (br. s, 1H), 7.36 (s, 1H), 7.11 (s, 1H), 4.03 (dd, J=7.6, 5.2 Hz, 1H), 3.01-2.89 (m, 2H), 2.28 (s, 3H), 2.25 (s, 3H); LCMS (ESI) m/z calc. for C₁₄H₁₇N₃O₂ 259.13. found, 260.1 [M+H]⁺.

### Preparation 42. (1R,2S,5S)—N-(1-Amino-3-(6,7-dimethyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.35)

To a solution of P1.34 (250 mg, 0.85 mmol, 1.1 eq), PA.4 (280 mg, 0.77 mmol, 1.0 eq) in butan-2-one (2.8 mL) was added 2-hydroxypyridine 1-oxide (HOPO, 21 mg, 0.19 mmol, 0.25 eq.) and 1-(3-dimethylaminopropyl)-3-ethylcarbodiimide hydrochloride (EDCI, 192 mg, 1.00 mmol, 1.3 eq.) at 0° C. After N,N-diisopropylethylamine (DIPEA, 536 μL, 3.08 mmol, 4.0 eq.) was added slowly to the reaction mixture, the resulting mixture was stirred at rt for 16 h. The mixture was concentrated under reduced pressure and then purified by C-18 reversed-phase column chromatography (0-100% MeOH in water containing 1% NH₄HCO₃) to give P1.35 (340 mg, 66% yield) as a yellow solid. ¹H NMR (400 MHz, DMSO-d₆), δ: 11.74-11.65 (m, 2H), 9.47-9.34 (m, 2H), 8.57-8.32 (m, 2H), 7.61-7.55 (m, 2H), 7.32-7.25 (m, 2H), 7.23-6.94 (m, 6H), 4.48-4.32 (m, 4H), 4.12-4.07 (m, 2H), 4.01-3.77 (m, 2H), 3.61-3.58 (m, 2H), 3.16-2.52 (m, 4H), 2.25-2.24 (m, 6H), 2.21-2.20 (m, 6H), 1.49-1.28 (m, 2H), 0.97-0.89 (m, 20H), 0.80-0.78 (m, 12H); LCMS (ESI) m/z calc. for C₃₀H₃₈F₃N₅O₅ 605.28. found, 606.4 [M+H]⁺.

### Synthesis of (1R,2S,5S)—N-(1-amino-i-oxo-3-(2-oxo-1,2-dihydropyridin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.41)

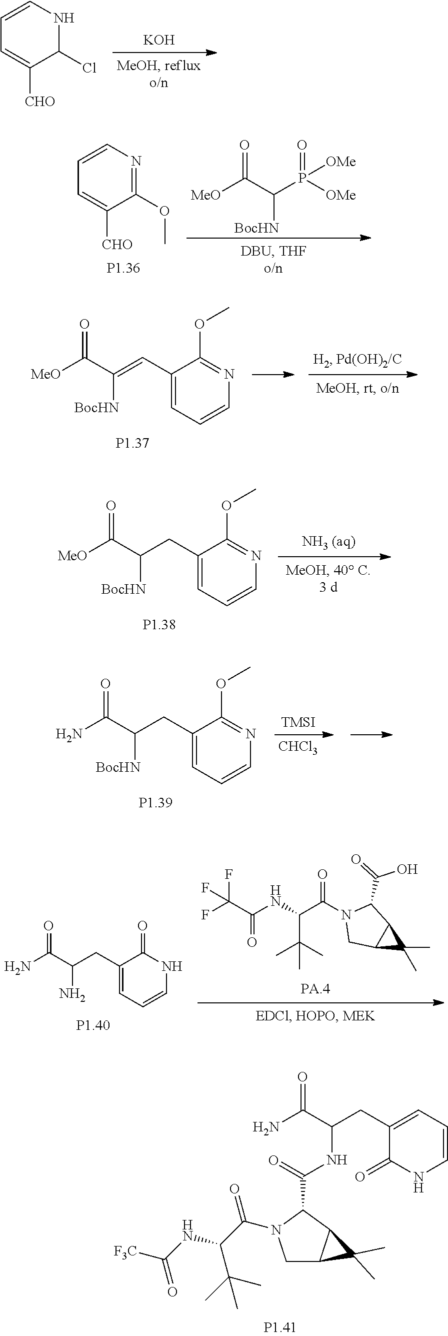

### Preparation 43. Dimethyl 2-methoxynicotinaldehyde (P1.36)

To a solution of 2-chloro-1,2-dihydropyridine-3-carbaldehyde (15.0 g, 105.97 mmol) in anh. MeOH (110 mL) was added potassium hydroxide (8.93 g, 160 mmol) at rt. The mixture was stirred at 70° C. overnight. The mixture was cooled and concentrated to remove solvent to give a crude. The reside was purified by silica-gel column chromatography (n-hexane:EtOAc=5:1) to get P1.36 (6.02 g, 41%) as yellow oil. ¹H NMR (400 MHz, CDCl₃), δ: 10.36 (br. s, 1H), 8.37 (dd, J=2.2, 4.8 Hz, 1H), 8.10 (dd, J=2.2, 7.6 Hz, 1H), 7.02-6.98 (m, 1H), 4.06 (s, 3H).

### Preparation 44. Methyl (Z)-2-((tert-butoxycarbonyl)amino)-3-(2-methoxypyridin-3-yl)acrylate (P1.37)

To a solution of methyl 2-((tert-butoxycarbonyl)amino)-2-(dimethoxyphosphoryl)acetate (963 mg, 1.4 mmol) in anh. THF (2.5 mL) was added 1,8-diazabicyclo[5.4.0]undec-7-ene (493 mg, 1.4 mmol) and P1.36 (317.6 mg, 2.32 mmol) at 0° C. The reaction was stirred at rt for overnight and concentrated to remove solvent. The residue was purified by silica-gel column chromatography (n-hexane/EtOAc=5:1) to give P1.37 (276 mg, 38% yield) as a yellow oil. ¹H NMR (400 MHz, CDCl₃), δ: 8.06 (dd, J=2.0, 4.8 Hz, 1H), 7.76 (d, J=7.6 Hz, 1H), 7.25 (s, 1H), 6.84-6.81 (m, 1H), 6.57 (s, 1H), 3.94 (s, 3H), 3.80 (s, 3H), 1.32 (s, 9H); LCMS (ESI) m/z calc. for C₁₅H₂₀N₂O₅ 308.14. found, 309.2 [M+H]⁺.

### Preparation 45. Methyl 2-((tert-butoxycarbonyl)amino)-3-(2-methoxypyridin-3-yl)propanoate (P1.38)

To a solution of P1.37 (276 mg, 0.89 mmol) in anh. methanol (5.0 mL) was added palladium hydroxide on carbon (27.6 mg, 10 wt %). The mixture was degassed with H_{2(g)} balloon two times. The reaction was stirred at rt under H_{2(g)} (1 atm) for overnight. It was filtered through a pad of celite and the celite pad was washed with EtOAc. The filtrate was concentrated to remove solvent to get the product which was used in next step without purification. ¹H NMR (400 MHz, CDCl₃): δ 8.09 (d, J=3.6 Hz, 1H), 7.42-7.40 (m, 1H), 6.87-6.84 (m, 1H), 5.15 (s, 1H), 4.57-4.55 (m, 1H), 4.00 (s, 3H), 3.72 (s, 3H), 3.12-3.07 (m, 1H), 3.00-2.94 (m, 1H), 1.37 (s, 9H).

### Preparation 46. tert-Butyl (1-amino-3-(2-methoxypyridin-3-yl)-1-oxopropan-2-yl)carbamate (P1.39)

To a solution of P1.38 (0.89 mmol) in methanol (3.0 mL) was added ammonium hydroxide solution (1.5 mL). The reaction was stirred at 40° C. for 3 days and concentrated to remove solvent to get the product (235 mg, 89%) as a yellow solid which was used in next step without further purification. ¹H NMR (400 MHz, CDCl₃), δ: 8.08-8.04 (m, 1H), 7.47-7.45 (m, 1H), 6.86-6.83 (m, 1H), 6.16 (br. s, 1H), 5.51 (br. s, 1H), 5.35 (d, J=7.6 Hz, 1H), 4.38 (br. s, 1H), 3.99 (s, 3H), 3.13-2.88 (m, 2H), 1.36 (s, 9H); LCMS (ESI) m/z calc. for C₁₄H₂₁N₃O₄ 295.15. found, 296.2 [M+H]⁺.

### Preparation 47. 2-Amino-3-(2-oxo-1,2-dihydropyridin-3-yl)propanamide (P1.40)

To a solution of P1.39 (1.0 g, 3.38 mmol) in anh. chloroform (50 mL) was added trimethylsilyl iodide (7.0 mL, 50.7 mmol). The reaction was stirred at 80° C. for overnight and cooled to rt. The solution was concentrated to remove solvent. The crude was purified by C18 reverse phase column chromatography (0-100% MeOH in water) and washed with diethyl ether to give P1.40 (411 mg, 67% yield) as a yellow oil. ¹H NMR (400 MHz, DMSO-d6), δ: 7.82 (br. s, 1H), 7.52 (br. s, 1H), 7.39-7.36 (m, 2H), 6.25-6.22 (m, 1H), 3.99-3.96 (m, 1H), 2.93-2.80 (m, 2H); LCMS (ESI) m/z calc. for C₈H₁₁N₃O₂ 181.09. found, 182.1 [M+H]⁺.

### Preparation 48. (1R,2S,5S)—N-(1-Amino-i-oxo-3-(2-oxo-1,2-dihydropyridin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.41)

To a solution of P1.40 (200 mg, 0.55 mmol), PA.4 (200 mg, 1.02 mmol) in butan-2-one (2.0 mL) was added 2-hydroxypyridine 1-oxide (2.0 mg, 0.14 mmol) and 1-(3-dimethylaminopropyl)-3-ethylcarbodiimide hydrochloride (138 mg, 0.72 mmol) at 0° C. After N,N-diisopropylethylamine (0.38 mL, 2.2 mmol) was added slowly to the reaction mixture, the resulting mixture was stirred for overnight and concentrated. The residue was purified by C₁₈-reverse-phase chromatography (0-80% MeOH in water) to give P1.41 (299 mg, 100%) as a white solid. ¹H NMR (400 MHz, MeOD-d4), δ: 7.49-7.33 (m, 1H), 7.24-7.02 (m, 2H), 6.95-6.93 (m, 2H), 6.40 (br. s, 1H), 6.28-6.25 (m, 1H), 5.65-5.59 (m, 2H), 4.61-4.52 (m, 4H), 4.43 (s, 2H), 4.33-4.04 (m, 2H), 3.92-3.79 (m, 4H), 3.12-3.02 (m, 2H), 1.57-1.52 (m, 4H), 1.06 (m, 24H), 0.85-0.84 (m, 6H); LCMS (ESI) m/z calc. for C₂₄H₃₂F₃N₅O₅ 527.24. found, 528.3 [M+H]⁺.

### Synthesis of (1R,2S,5S)—N-[2-amino-2-oxo-1-[(3-oxo-4H-quinoxalin-2-yl)methyl]ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide

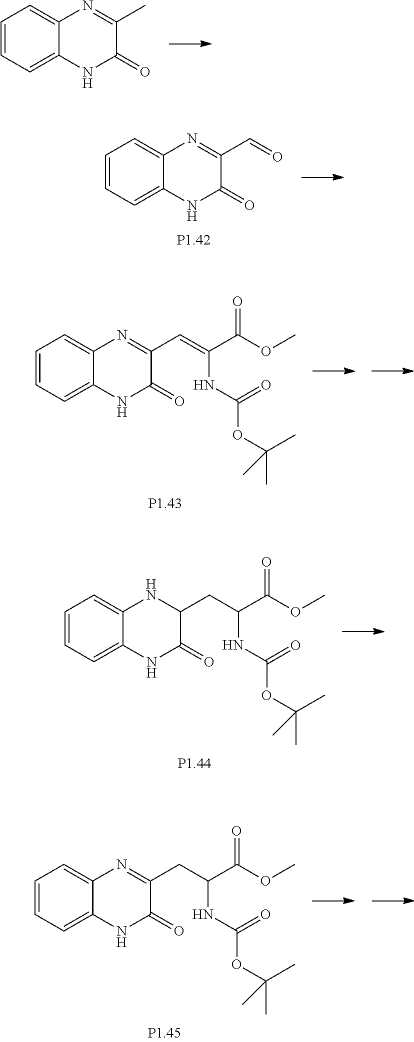 [SMILES: Cc1nc2ccccc2[nH]c1=O ; O=Cc1nc2ccccc2[nH]c1=O ; COC(=O)/C(=C/c1nc2ccccc2[nH]c1=O)NC(=O)OC(C)(C)C ; COC(=O)C(CC1Nc2ccccc2NC1=O)NC(=O)OC(C)(C)C ; COC(=O)C(Cc1nc2ccccc2[nH]c1=O)NC(=O)OC(C)(C)C]

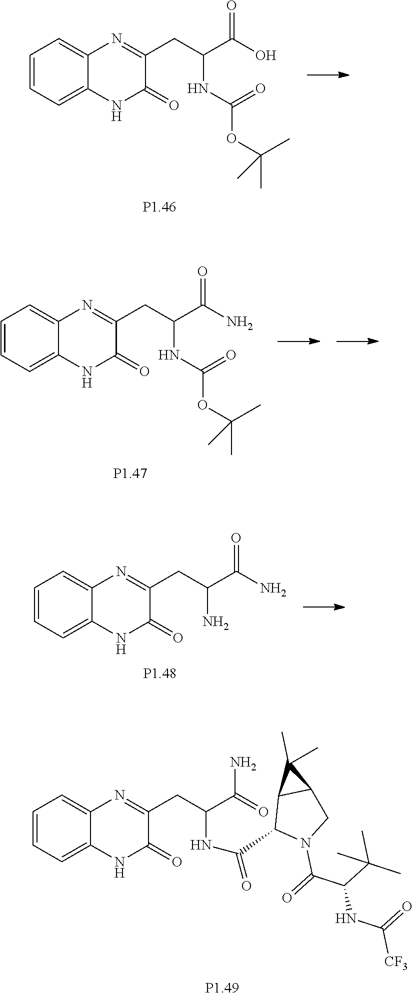 [SMILES: CC(C)(C)OC(=O)NC(Cc1nc2ccccc2[nH]c1=O)C(=O)O ; CC(C)(C)OC(=O)NC(Cc1nc2ccccc2[nH]c1=O)C(N)=O ; NC(=O)C(N)Cc1nc2ccccc2[nH]c1=O ; CC(C)(C)[C@H](NC(=O)C(F)(F)F)C(=O)N1C[C@H]2[C@@H]([C@H]1C(=O)NC(Cc1nc3ccccc3[nH]c1=O)C(N)=O)C2(C)C]

### Preparation 49. 3-Oxo-4H-quinoxaline-2-carbaldehyde (P1.42)

A mixture of 3-methyl-1H-quinoxalin-2-one (5 g, 31.2 mmol) and selenium dioxide (7.28 g, 65.5 mmol) in dry dioxane (100 mL) was stirred at reflux under nitrogen for 30 min. After cooling, the dark-brown mixture was concentrated under reduced pressure and the residue was purified by FC (AcOEt) to give the title compound P1.42 as an orange solid. 4.4 g, 80%.

### Preparation 50. Methyl (Z)-2-(tert-butoxycarbonylamino)-3-(3-oxo-4H-quinoxalin-2-yl)prop-2-enoate (P1.43)

To a solution of (±)-Boc-α-phosphonoglycine trimethyl ester (8.26 g, 27.7 mmol) in anh. THF (20 mL) was added 1,8-diazabicyclo[5.4.0]undec-7-ene (4.23 g, 27.7 mmol) slowly at 0° C. After the reaction was stirred at 0° C. for 30 min, the ylide solution was added slowly into a solution of 3-oxo-4H-quinoxaline-2-carbaldehyde (P1.42, 4.4 g, 25.2 mmol) in anh. THF (10 mL) at 0° C. The mixture was warmed to rt and stirred overnight. The solvent was removed under reduced pressure. The residue was purified by silica gel column chromatography (n-hexane/EtOAc 2:1, next CHCl₃:EtOAc 3:1) to give title compound P1.43 (2.65 g, 30%). ¹H NMR (400 MHz, DMSO), δ: 12.55 (s, 1H), 10.93 (s, 1H), 7.65 (dd, J=8.0, 1.4 Hz, 1H), 7.57-7.48 (m, 1H), 7.38-7.26 (m, 2H), 6.63 (s, 1H), 3.81 (s, 3H), 1.46 (s, 9H).

### Preparation 51. Methyl 2-(tert-butoxycarbonylamino)-3-(3-oxo-2,4-dihydro-1H-quinoxalin-2-yl)propanoate (P1.44)

To a solution of methyl (Z)-2-(tert-butoxycarbonylamino)-3-(3-oxo-4H-quinoxalin-2-yl)prop-2-enoate (P1.43, 2.65 g, 7.7 mmol) in methanol (50 mL) was added Pd/C (0.27 g, 10% wt). The reaction was stirred overnight under hydrogen atmosphere (5 MPa). The solution was filtered through a pad of celite. The filtrate was concentrated under reduced pressure to give title compound (2.6 g, 98%) as a solid. ¹H NMR (400 MHz, DMSO), δ: 10.94 (s, 1H), 9.68 (s, 1H), 8.77-8.68 (m, 2H), 8.48 (s, 1H), 8.38-8.28 (m, 1H), 8.22 (dd, J=16.6, 2.2 Hz, 1H), 7.60-7.50 (m, 1H), 7.13 (d, J=8.1 Hz, 1H), 7.06 (d, J=9.5 Hz, 2H), 6.74 (dd, J=16.8, 2.7 Hz, 1H), 5.05 (dd, J=13.2, 5.2 Hz, 1H), 4.33 (d, J=16.9 Hz, 1H), 4.28-4.17 (m, 1H), 3.90-3.81 (m, 1H), 3.67 (s, 1H), 3.60 (s, 3H), 3.03 (t, J=7.5 Hz, 2H), 2.88 (d, J=10.1 Hz, OH), 2.58 (d, J=17.0 Hz, 1H), 2.37 (d, J=7.6 Hz, 1H), 1.82 (dd, J=15.7, 8.1 Hz, 3H), 1.72-1.66 (m, 2H), 1.57 (d, J=7.8 Hz, 2H), 1.44-1.38 (m, 3H), 1.30-1.18 (m, 2H).

### Preparation 52. Methyl 2-(tert-butoxycarbonylamino)-3-(3-oxo-4H-quinoxalin-2-yl)propanoate (P1.45)

To a solution of methyl 2-(tert-butoxycarbonylamino)-3-(3-oxo-2,4-dihydro-1H-quinoxalin-2-yl)propanoate (P1.44, 2.6 g, 7.4 mmol) in DCM (50 mL) was added MnO₂ (1.94 g, 22.2 mmol). The reaction was stirred overnight. The solution was filtered through a pad of celite. The filtrate was concentrated under reduced pressure to give title compound P1.45 (2.3 g, 89%) as a solid. ¹H NMR (400 MHz, DMSO), δ: 12.35 (s, 1H), 7.67 (d, J=7.9 Hz, 1H), 7.48 (t, J=7.7 Hz, 1H), 7.28 (d, J=7.8 Hz, 3H), 4.66 (t, J=7.5 Hz, 1H), 3.59 (s, 3H), 3.31 (s, 3H), 2.99 (dd, J=14.7, 8.3 Hz, 1H), 1.39 (s, 2H), 1.27 (d, J=3.1 Hz, 9H).

### Preparation 53. 2-(tert-Butoxycarbonylamino)-3-(3-oxo-4H-quinoxalin-2-yl)propanoic acid (P1.46)

A mixture of methyl 2-(tert-butoxycarbonylamino)-3-(3-oxo-4H-quinoxalin-2-yl)propanoate (P1.45, 2.3 g, 6.6 mmol), NaOH (0.4 g, 9.9 mmol) in 20 mL MeOH and 5 mL H₂O was stirred at 60° C. for 14 h and evaporated. The residue was diluted with water and acidified with 6M HCl to pH=3-4. The formed solid was filtered off and dried. Yield of title compound P1.46 (2.2 g, 99%). ¹H NMR (400 MHz, DMSO), δ: 12.31 (s, 2H), 7.68 (d, J=8.0 Hz, 1H), 7.47 (s, 1H), 7.27 (d, J=8.1 Hz, 2H), 7.10 (d, J=8.5 Hz, 1H), 4.60 (d, J=7.7 Hz, 1H), 2.98-2.88 (m, 1H), 1.39 (s, 1H), 1.25 (s, 10H).

### Preparation 54. tert-Butyl N-[2-amino-2-oxo-1-[(3-oxo-4H-quinoxalin-2-yl)methyl]ethyl]carbamate (P1.47)

To a mixture of the 2-(tert-butoxycarbonylamino)-3-(3-oxo-4H-quinoxalin-2-yl)propanoic acid (P1.46, 2.2 g, 6.6 mmol) and HATU (2.63 g, 6.9 mmol) in DMF (10 ml) DIEA (0.98 g, 7.6 mmol) was added. The mixture was stirred at rt for 30 min, and ammonium carbonate (1.27 g, 13.2 mmol) was added, and stirred overnight. Reaction mixture was diluted with ethyl acetate (50 ml) and washed with water (3*50 ml), brine (2×50 mL), dried on Na₂SO₄, and concentrated under reduced pressure. Yield of title P1.47 compound (1.46 g, 67%). ¹H NMR (400 MHz, DMSO), δ: 12.28 (s, 1H), 7.67 (d, J=8.0 Hz, 1H), 7.46 (t, J=7.7 Hz, 1H), 7.30-7.22 (m, 3H), 7.05 (s, 1H), 6.87 (d, J=8.6 Hz, 1H), 4.50 (s, 1H), 3.26 (d, J=5.1 Hz, 1H), 2.87 (dd, J=14.4, 9.4 Hz, 1H), 1.21 (d, J=2.5 Hz, 8H).

### Preparation 55. 2-Amino-3-(3-oxo-4H-quinoxalin-2-yl)propanamide (P1.48)

To a solution of tert-butyl N-[2-amino-2-oxo-1-[(3-oxo-4H-quinoxalin-2-yl)methyl]ethyl]carbamate (1.46 g, 4.4 mmol) in dioxane (10 mL) HCl (30 mL, 3M in dioxane) was added. The reaction was stirred at 50° C. for overnight. The solvent was removed under reduced pressure. Yield of title compound P1.48 (1.17 g, 99%). ¹H NMR (400 MHz, DMSO), δ: 12.58 (s, 1H), 8.34 (d, J=5.1 Hz, 3H), 7.98 (s, 1H), 7.78 (dd, J=8.1, 1.4 Hz, 1H), 7.59-7.47 (m, 2H), 7.38 (d, J=8.2 Hz, 1H), 7.34-7.28 (m, 1H), 4.34 (d, J=6.4 Hz, 1H), 3.32 (d, J=6.5 Hz, 2H).

### Preparation 56. (1R,2S,5S)—N-[2-amino-2-oxo-1-[(3-oxo-4H-quinoxalin-2-yl)methyl]ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (P1.49)

To a mixture of the (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid (PA.4, 1.68 g, 4.62 mmol) and HATU (1.84 g, 4.84 mmol) in MeCN (20 ml) DIEA (1.42 g, 11 mmol) was added. The mixture was stirred at rt for 30 min, and 2-amino-3-(3-oxo-4H-quinoxalin-2-yl)propanamide (P1.48, 1.17 g, 4.4 mmol) was added, and stirred overnight. Reaction mixture was diluted with ethyl acetate (50 ml) and washed with water (3×50 ml), brine (2×50 mL), dried on Na₂SO₄, and concentrated under reduced pressure. The residue was purified by silica gel column chromatography (EtOAc, next EtOAc:MeOH 100:2) to give title compound P1.49 as inseparable mixture diastereomers (2.07 g, 81%). ¹H NMR (400 MHz, DMSO), δ: 12.34 (d, J=14.0 Hz, 1H), 9.36 (dd, J=15.9, 8.3 Hz, 1H), 8.54 (d, J=8.5 Hz, 1H), 8.18 (d, J=7.7 Hz, 1H), 7.73-7.61 (m, 2H), 7.47 (t, J=7.8 Hz, 2H), 7.26 (dd, J=13.4, 7.7 Hz, 5H), 7.10 (d, J=4.3 Hz, 2H), 6.99 (s, 1H), 4.84 (d, J=12.3 Hz, 1H), 4.71 (q, J=7.3 Hz, 1H), 4.40 (d, J=8.4 Hz, 1H), 4.34 (d, J=8.1 Hz, 1H), 4.14 (d, J=9.3 Hz, 2H), 3.89 (dd, J=10.6, 5.4 Hz, 1H), 3.79 (dd, J=10.5, 5.6 Hz, 1H), 3.40 (dd, J=14.9, 4.8 Hz, 1H), 3.25 (dd, J=15.3, 6.1 Hz, 1H), 3.13-3.02 (m, 2H), 2.91 (dd, J=15.3, 9.5 Hz, 1H), 1.45 (t, J=6.6 Hz, 1H), 1.35-1.21 (m, 2H), 1.10 (q, J=1.2 Hz, 4H), 1.05-0.88 (m, 21H), 0.88-0.72 (m, 10H).

### Synthesis of (1R,2S,5S)—N-[2-amino-1-[(1-methyl-3-oxo-2,4-dihydroquinoxalin-2-yl)methyl]-2-oxo-ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (P1.54)

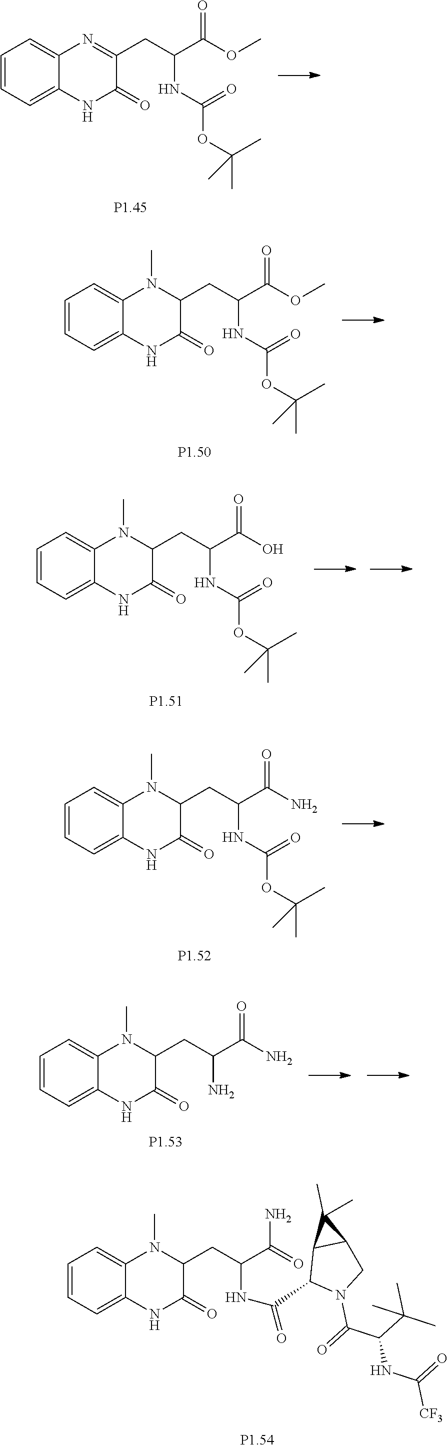 [SMILES: COC(=O)C(Cc1nc2ccccc2[nH]c1=O)NC(=O)OC(C)(C)C ; COC(=O)C(CC1C(=O)Nc2ccccc2N1C)NC(=O)OC(C)(C)C ; CN1c2ccccc2NC(=O)C1CC(NC(=O)OC(C)(C)C)C(=O)O ; CN1c2ccccc2NC(=O)C1CC(NC(=O)OC(C)(C)C)C(N)=O ; CN1c2ccccc2NC(=O)C1CC(N)C(N)=O ; CN1c2ccccc2NC(=O)C1CC(NC(=O)[C@@H]1[C@@H]2[C@H](CN1C(=O)[C@@H](NC(=O)C(F)(F)F)C(C)(C)C)C2(C)C)C(N)=O]

### Preparation 57. Methyl 2-(tert-butoxycarbonylamino)-3-(1-methyl-3-oxo-2,4-dihydroquinoxalin-2-yl)propanoate (P1.50)

To a solution of P1.45 (325 mg, 0.9 mmol) and formalin (1 ml) in methanol (30 mL) was added Pd/C (33 mg, 10% wt). The reaction was stirred overnight under hydrogen atmosphere (5 MPa). The solution was filtered through a pad of celite. The filtrate was concentrated under reduced pressure to give title compound P1.50 (340 mg, 98%) as a solid.

### Preparation 58. 2-(tert-Butoxycarbonylamino)-3-(1-methyl-3-oxo-2,4-dihydroquinoxalin-2-yl)propanoic acid (P1.51)

A mixture of P1.50 (330 mg, 0.9 mmol), NaOH (72 mg, 1.8 mmol) in 10 mL MeOH and 3 mL H₂O was stirred at 60° C. for 14 h and evaporated. The residue was diluted with water and acidified with 6M HCl to pH=5-6, extracted with CHCl₃ (3×50 ml) dried with Na₂SO₄, and concentrated under reduced pressure, and the crude was used in next step without purification.

### Preparation 59. tert-Butyl N-[2-amino-i-[(1-methyl-3-oxo-2,4-dihydroquinoxalin-2-yl)methyl]-2-oxo-ethyl]carbamate (P1.52)

To a mixture of the P1.51 (320 mg, 0.9 mmol) and HATU (383 mg, 1 mmol) in DMF (5 ml) DIEA (154 mg, 1.2 mmol) was added. The mixture was stirred at rt for 30 min, and ammonium carbonate (176 mg, 1.8 mmol) was added, and stirred overnight. Reaction mixture was diluted with ethyl acetate (50 ml) and washed with water (3×50 ml), brine (2×50 mL), dried on Na₂SO₄, and concentrated under reduced pressure, and the crude was used in next step without purification.

### Preparation 60. 2-Amino-3-(1-methyl-3-oxo-2,4-dihydroquinoxalin-2-yl)propanamide (P1.53)

To a solution of P1.52 (300 mg, 0.86 mmol) in dioxane (10 mL) HCl (30 mL, 3M in dioxane) was added. The reaction was stirred at 50° C. for overnight. The solvent was removed under reduced pressure, and the crude was used in next step without purification.

### Preparation 61. (1R,2S,5S)—N-[2-amino-1-[(1-methyl-3-oxo-2,4-dihydroquinoxalin-2-yl)methyl]-2-oxo-ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (P1.54)

To a mixture of the (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid (PA.4, 313 mg, 0.86 mmol) and HATU (360 mg, 0.95 mmol) in MeCN (10 ml) DIEA (445 mg, 3.44 mmol) was added. The mixture was stirred at rt for 30 min, and P1.53 (276 mg, 0.86 mmol) was added, and stirred overnight. Reaction mixture was diluted with ethyl acetate (50 ml) and washed with water (3×50 ml), brine (2×50 mL), dried on Na₂SO₄, and concentrated under reduced pressure, and the crude was used in next step without purification.

### Synthesis of (1R,2S,5S)—N-(1-amino-3-(6-methyl-2-oxo-1,2-dihydroquinolin-3-yl)-1-oxopropan-2-yl)-3-((S)-3,3-dimethyl-2-((4-methylthiazol-2-yl)amino)butanoyl)-6,6-dimethyl-3-azabicyclo[3.1.0]hexane-2-carboxamide (P1.59)

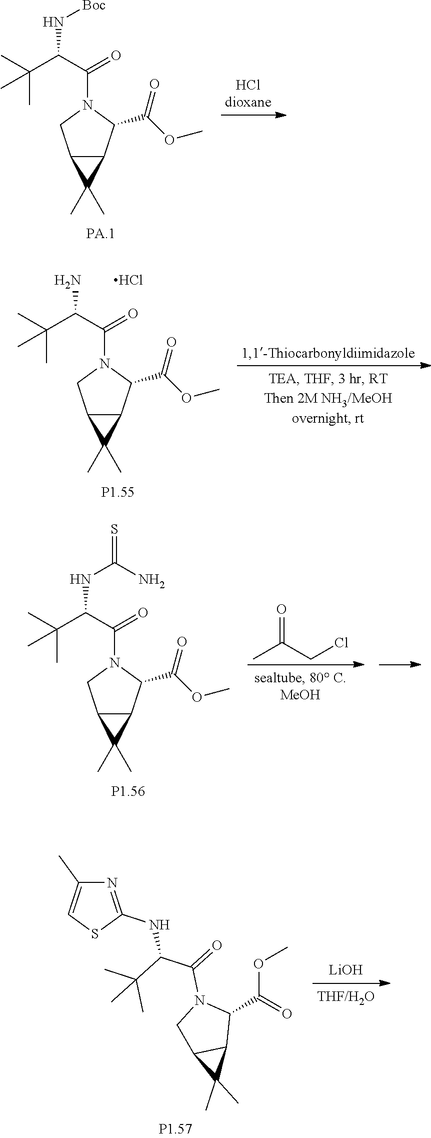

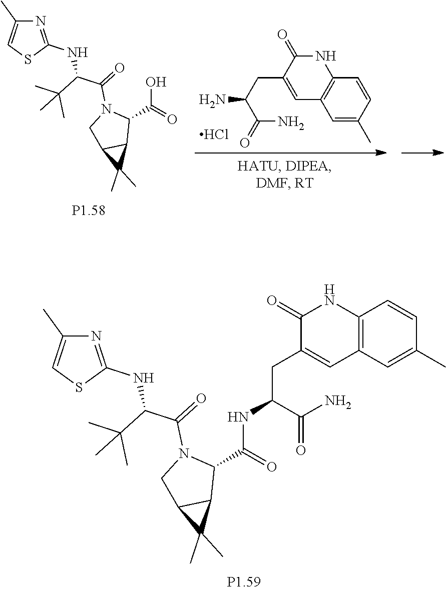

### Preparation 62. Methyl (1R,2S,5S)-3-((S)-3,3-dimethyl-2-((4-methylthiazol-2-yl)amino)butanoyl)-6,6-dimethyl-3-azabicyclo[3.1.0]hexane-2-carboxylate (P1.57)

To a solution of P1.56 (600 mg, 1.76 mmol, 1.0 eq) in methanol (70 mL) was added 1-chloropropan-2-one (353 L, 4.40 mmol, 2.5 eq) slowly at rt. The reaction mixture was heated at 80° C. for 3 days. The reaction mixture was concentrated to get crude product. The residue was purified by silica gel flash column chromatography (2-3% MeOH in DCM) to give P1.57 (500 mg, 74% yield) as a white solid. ¹H NMR (400 MHz, MeOD-d₄), δ: 6.13 (s, 1H), 4.49 (s, 1H), 4.33 (s, 1H), 4.16 (d, J=10.4 Hz, 1H), 4.00-3.97 (m, 1H), 3.74 (s, 3H), 2.16 (s, 3H), 1.60 (dd, J=7.6, 5.6 Hz, 1H), 1.46 (d, J=7.6 Hz, 1H), 1.10 (s, 9H), 1.05 (s, 3H), 0.85 (s, 3H).

### Preparation 63. (1R,2S,5S)-3-((S)-3,3-Dimethyl-2-((4-methylthiazol-2-yl)amino)butanoyl)-6,6-dimethyl-3-azabicyclo[3.1.0]hexane-2-carboxylic acid (P1.58)

To a solution of P1.57 (100 mg, 0.263 mmol, 1.0 eq) in THF (0.53 mL) was added a solution of LiOH (18.9 mg, 0.79 mmol, 3.0 eq) in water (0.53 mL). The resulting mixture was stirred at rt for overnight. The mixture was neutralized with 1N HCl(aq.) and extracted with ethyl acetate. The combined organic layers were dried over Na₂SO₄₍ₛ₎, filtered, and concentrated in vacuo to give P1.58 (70 mg, 72% yield) as a white solid. ¹H NMR (400 MHz, MeOD-d₄), δ: 6.16 (s, 1H), 4.47 (s, 1H), 4.32 (s, 1H), 4.13 (d, J=10.8 Hz, 1H), 4.00-3.96 (m, 1H), 2.17 (s, 3H), 1.59 (dd, J=7.6, 5.2 Hz, 1H), 1.50 (d, J=7.6 Hz, 1H), 1.10 (s, 9H), 1.06 (s, 3H), 0.86 (s, 3H).

### Preparation 64. (1R,2S,5S)—N-(1-Amino-3-(6-methyl-2-oxo-1,2-dihydroquinolin-3-yl)-1-oxopropan-2-yl)-3-((S)-3,3-dimethyl-2-((4-methylthiazol-2-yl)amino)butanoyl)-6,6-dimethyl-3-azabicyclo[3.1.0]hexane-2-carboxamide (P1.59)

To a solution of P1.58 (100 mg, 0.2736 mmol, 1.0 eq) in DMF (1.47 mL) was added (S)-2-amino-3-(6-methyl-2-oxo-1,2-dihydroquinolin-3-yl)propanamide hydrochloride (93 mg, 0.3283 mmol, 1.2 eq), (1-[bis(dimethylamino)methylene]-1H-1,2,3-triazolo[4,5-b]pyridinium 3-oxide hexafluorophosphate (HATU, 114 mg, 0.30 mmol, 1.1 eq) and diisopropyl ethyl amine (DIPEA, 142.9 μL, 0.821 mmol, 3.0 eq). The resulting mixture was stirred at rt for overnight. The mixture was directly purified with C-18 reversed-phase column chromatography (mobile phase A: water, mobile phase B: methanol, UV: 214 and 254 nm, Flow rate: 35 mL/min, Gradient: 70-80% (% B)) to give P1.59 (40 mg, 24% yield) as a white solid. ¹H NMR (400 MHz, MeOD-d₄), δ: 7.79 (s, 1H), 7.40 (s, 1H), 7.33-7.30 (m, 1H), 7.20 (d, J=8.0 Hz, 1H), 6.05 (s, 1H), 4.59-4.56 (m, 1H), 4.46 (s, 1H), 4.12-4.09 (m, 3H), 3.27-3.03 (m, 2H), 2.36 (s, 3H), 2.12 (s, 3H), 1.56-1.52 (m, 1H), 1.19 (d, J=7.6 Hz, 1H), 0.98 (s, 9H), 0.89 (s, 3H), 0.75 (s, 3H).

### Synthesis of (1R,2S,5S)—N-(1-amino-3-(6-methyl-2-oxo-1,2-dihydroquinolin-3-yl)-1-oxopropan-2-yl)-3-((S)-3,3-dimethyl-2-((4-(trifluoromethyl)pyrimidin-2-yl)amino)butanoyl)-6,6-dimethyl-3-azabicyclo[3.1.0]hexane-2-carboxamide (P1.64)

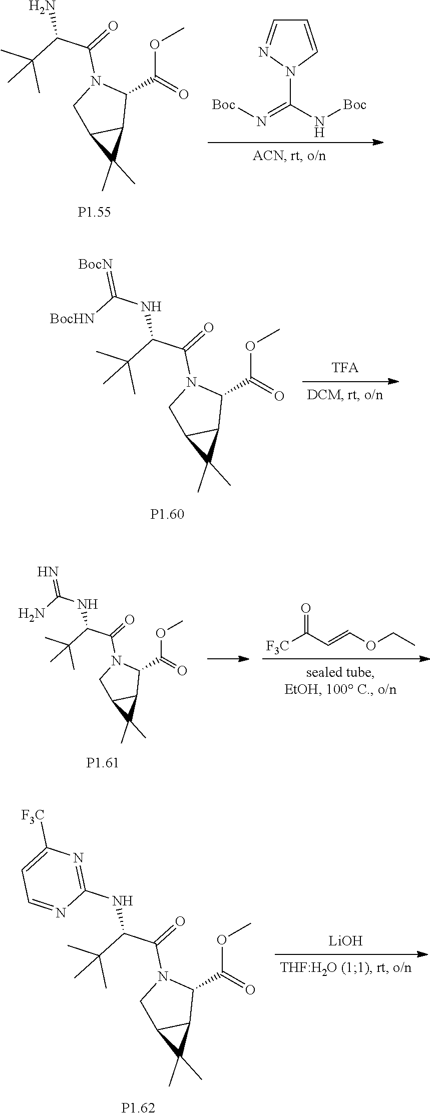

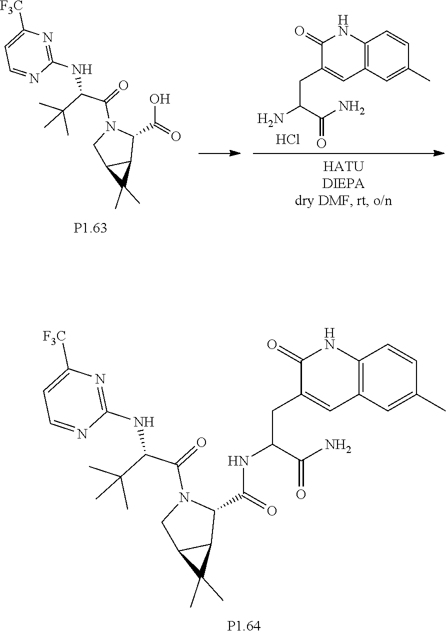 [SMILES: CC(C)(C)[C@H](Nc1nccc(C(F)(F)F)n1)C(=O)N1C[C@H]2[C@@H]([C@H]1C(=O)O)C2(C)C ; Cc1ccc2[nH]c(=O)c(CC(N)C(N)=O)cc2c1 ; Cl ; Cc1ccc2[nH]c(=O)c(CC(NC(=O)[C@@H]3[C@@H]4[C@H](CN3C(=O)[C@@H](Nc3nccc(C(F)(F)F)n3)C(C)(C)C)C4(C)C)C(N)=O)cc2c1]

### Preparation 65. Methyl (1R,2S,5S)-3-[(2S)-2-[[(Z)—N,N′-bis(tert-butoxycarbonyl)carbamimidoyl]amino]-3,3-dimethyl-butanoyl]-6,6-dimethyl-3-azabicyclo[3.1.0]hexane-2-carboxylate (P1.60)

To a solution of methyl (1R,2S,5S)-3-((S)-2-amino-3,3-dimethylbutanoyl)-6,6-dimethyl-3-azabicyclo[3.1.0]hexane-2-carboxylate (P1.55, 750 mg, 2.66 mmol) in anh. acetonitrile (10 mL) was added N,N′-di-Boc-1H-pyrazole-1-carboxamidine (1071 mg, 3.45 mmol). The reaction was stirred at rt for overnight. The solvents were evaporated and the crude mixture was purified by silica gel column chromatography (0-70% DCM in hexane to wash out unreacted reagent and 0-30% EtOAc in hexane to elute desired product) to give P1.60 (480 mg, 40% yield) as a white foam. ¹H NMR (400 MHz, CDCl₃), δ: 11.33 (s, 1H), 8.85 (d, J=8.8 Hz, 1H), 4.57 (d, J=8.4 Hz, 1H), 4.44 (s, 1H), 4.36 (J=10.0 Hz, 1H), 3.91-3.89 (m, 1H), 3.74 (s, 3H), 1.50-1.49 (m, 1H), 1.47 (s, 18H), 1.43-1.42 (m, 1H), 1.10 (s, 9H), 1.05 (s, 3H), 0.98 (s, 3H).

### Preparation 66. Methyl (1R,2S,5S)-3-((S)-2-guanidino-3,3-dimethylbutanoyl)-6,6-dimethyl-3-azabicyclo[3.1.0]hexane-2-carboxylate (P1.61)

To the solution of P1.60 (480 mg, 0.914 mmol) in DCM (22 mL) was added trifluoroacetic acid (5.6 mL) dropwise at 0° C. and stirred at rt overnight. The mixture was concentrated to give P1.61 (542 mg, quantitative) as a brown gum which was directly used in the next step without further purification. ¹H NMR (400 MHz, MeOD-d₄), δ: 7.96 (d, J=2.4 Hz 1H), 8.38 (d, J=10.4 Hz, 1H), 4.42 (s, 1H), 4.23-4.20 (m, 1H), 3.95-3.91 (m, 1H), 3.85 (d, J=10.8 Hz, 1H), 3.75 (s, 3H), 1.61-1.52 (m, 2H), 1.08 (s, 12H), 0.92 (m, 3H).

### Preparation 67. Methyl (1R,2S,5S)-3-((S)-3,3-dimethyl-2-((4-(trifluoromethyl)pyrimidin-2-yl)amino)butanoyl)-6,6-dimethyl-3-azabicyclo[3.1.0]hexane-2-carboxylate (P1.62)

To a solution of P1.61 (200 mg, 0.475 mmol) in ethanol (8 mL) was added (E)-4-ethoxy-1,1,1-trifluorobut-3-en-2-one at rt (332 mg, 2.375 mmol). The reaction was stirred overnight in a sealed tube at 100° C. The solvents were evaporated under reduced pressure and crude reaction mixture was purified by silica gel column chromatography (0-30% ethyl acetate in hexane) to give P1.62 (135 mg, 66%) as a brown solid. ¹H NMR (400 MHz, MeOH-d₄), δ: 8.53 (d, J=4.8 Hz 1H), 6.95 (d, J=4.8 Hz, 1H), 4.62 (br. s, 1H), 4.34 (s, 1H), 4.20 (d, J=10.4 Hz, 1H), 4.11-4.01 (m, 1H), 3.74 (s, 3H), 1.63-1.59 (m, 1H), 1.49-1.47 (m, 1H), 1.12-0.94 (m, 15H).

### Preparation 68. (1R,2S,5S)-3-((S)-3,3-Dimethyl-2-((4-(trifluoromethyl)pyrimidin-2-yl)amino)butanoyl)-6,6-dimethyl-3-azabicyclo[3.1.0]hexane-2-carboxylic acid (P1.63)

A solution of P1.62 (135 mg, 0.315 mmol) in THF (2.0 mL) was added to a solution of LiOH (22.6 mg, 0.945) in water (2.0 mL) and then resulting mixture was stirred at room temperature for overnight. The solvents were evaporated to obtain brown syrup P1.63 (176 mg, quantitative yield) as a crude product which was used in next step without further purification. ¹H NMR (400 MHz, MeOD-d₄), δ: 8.54 (d, J=5.0 Hz 1H), 6.94 (d, J=5.0 Hz, 1H), 4.68 (br. s, 1H), 4.22 (s, 1H), 4.14-4.04 (m, 2H), 1.88-1.86 (m, 1H), 1.28-1.12 (m, 1H), 1.13-0.86 (m, 15H).

### Preparation 69. (1R,2S,5S)—N-(1-Amino-3-(6-methyl-2-oxo-1,2-dihydroquinolin-3-yl)-1-oxopropan-2-yl)-3-((S)-3,3-dimethyl-2-((4-(trifluoromethyl)pyrimidin-2-yl)amino)butanoyl)-6,6-dimethyl-3-azabicyclo[3.1.0]hexane-2-carboxamide (P1.64)

To a solution of P1.63 (176 mg, 0.425 mmol) in DMF (4.0 mL) was added 2-amino-3-(6-methyl-2-oxo-1,2-dihydroquinolin-3-yl)propanamide (144 mg, 0.510 mmol), HATU (339 mg, 0.892 mmol) and diisopropyl ethyl amine (DIPEA, 370 μL, 2.125 mmol) then resulting mixture stirred at rt for overnight. The mixture directly was purified with C-18 reversed-phase column chromatography (0-100% MeOH in water) to obtain desired product P1.64 (77 mg, 28% yield) as a yellow solid. ¹H NMR (400 MHz, MeOD-d₄), δ: 8.53-8.52 (m, 2H), 7.81 (s, 1H), 7.75 (s, 1H), 7.42-7.20 (m, 6H), 6.95-6.94 (m, 2H), 4.81-4.77 (m, 2H), 4.63-4.60 (m, 2H), 4.12-3.97 (m, 4H), 3.38-3.32 (m, 2H), 3.08-2.79 (m, 2H), 2.39 (s, 3H), 2.37 (s, 3H), 1.39-1.35 (m, 2H), 1.29-1.24 (m, 1H), 1.05-0.85 (m, 31H).

### Synthesis of 3-[3,3-Dimethyl-2-(2-methyl-pyrimidin-4-ylamino)-butyryl]-6,6-dimethyl-3-aza-bicyclo[3.1.0]hexane-2-carboxylic acid [1-carbamoyl-2-(6-methyl-2-oxo-1,2-dihydro-quinolin-3-yl)-ethyl]-amide (P1.67)

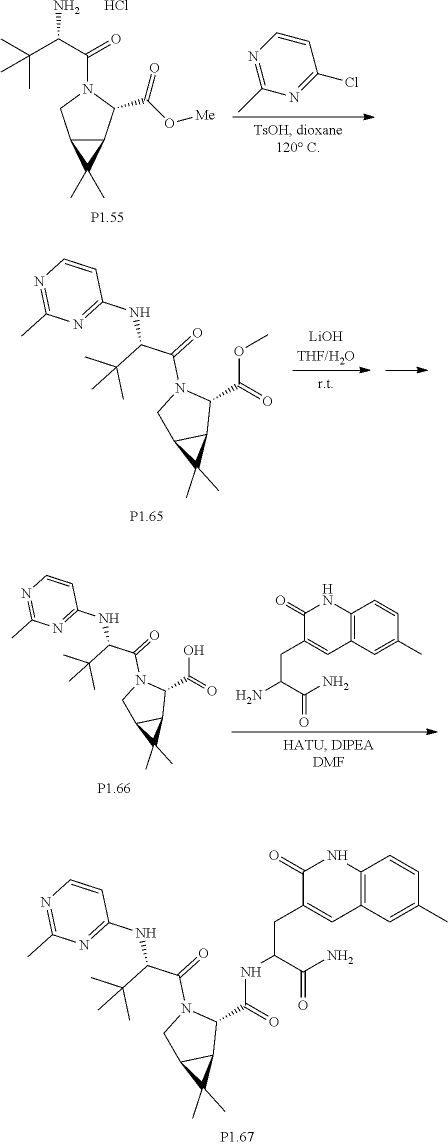

### Preparation 70. 3-[3,3-Dimethyl-2-(2-methyl-pyrimidin-4-ylamino)-butyryl]-6,6-dimethyl-3-aza-bicyclo[3.1.0]hexane-2-carboxylic acid methyl ester (P1.65)

To a solution of 3-(2-Amino-3,3-dimethyl-butyryl)-6,6-dimethyl-3-aza-bicyclo[3.1.0]hexane-2-carboxylic acid methyl ester (P1.55, 1.0 g, 3.1 mmol, 1.0 eq) in dioxane was added 4-chloro-2-methyl-pyrimidine (0.80 g, 6.2 mmol, 2.0 eq) and p-toluenesulfonic acid (PTSA, 0.5 g, 3.1 mmol, 1.0 eq) and the reaction mixture was stirred at 120° C. for 16 h. The mixture was concentrated to remove dioxane. The residue was purified by C18 reverse phase column chromatography to give P1.65 (203.8 mg, 17% yield) as a gum. ¹H NMR (400 MHz, CDCl₃), δ: 7.93 (d, J=6.2 Hz, 1H), 6.24 (d, J=6.2 Hz, 1H), 5.96 (d, J=10.0 Hz, 1H), 4.89 (br. s, 1H), 4.37 (s, 1H), 4.13 (d, J=10.0 Hz, 1H), 3.99-3.95 (m, 1H), 3.72 (s, 3H), 2.47 (s, 3H), 1.51-1.48 (m, 1H), 1.42 (d, J=7.6 Hz, 1H), 1.06 (s, 9H), 1.01 (s, 3H), 0.75 (s, 3H).

### Preparation 71. 3-[3,3-Dimethyl-2-(2-methyl-pyrimidin-4-ylamino)-butyryl]-6,6-dimethyl-3-aza-bicyclo[3.1.0]hexane-2-carboxylic acid (P1.66)

A solution of P1.65 (203.8 mg, 0.50 mmol, 1.0 eq) in THF (11 mL) was added to a solution of LiOH (39.1 mg, 1.6 mmol, 3.0 eq) in water (11 mL). The resulting mixture was stirred at rt for overnight. The mixture was concentrated to remove solvent to give crude P1.66 (210.0 mg, >99% yield) as a white solid. LCMS (ESI) m/z calc. for C₁₉H₂₈N₄O₃ 360.45. found, 361.1 [M+H]⁺.

### Preparation 72. (1R,2S,5S)—N-[2-Amino-1-[(6-methyl-2-oxo-1H-quinolin-3-yl)methyl]-2-oxo-ethyl]-3-[(2S)-3,3-dimethyl-2-[(2-methylpyrimidin-4-yl)amino]butanoyl]-6,6-dimethyl-3-azabicyclo[3.1.0]hexane-2-carboxamide (P1.67)

To a solution of P1.66 (120.0 mg, 0.3 mmol, 1.0 eq) in DMF (2.5 mL) was added 2-amino-3-(6-methyl-2-oxo-1,2-dihydroquinolin-3-yl)propanamide (113.0 mg, 0.40 mmol, 1.2 eq), HATU (140 mg, 0.366 mmol, 1.1 eq) and DIPEA (0.2 mL, 1.0 mmol, 3.0 eq). The resulting mixture was stirred at rt for overnight. The mixture was directly purified with C-18 reverse phase column chromatography to give P1.67 (125.0 mg, 64% yield) as a white solid. ¹H NMR (400 MHz, CD₃OD), δ: 7.88-7.85 (m, 1H), 7.79-7.75 (m, 1H), 7.39-7.37 (m, 1H), 7.33-7.29 (m, 1H), 7.23-7.19 (m, 1H), 6.48-6.44 (m, 1H), 4.99-4.97 (m, 1H), 4.84-4.62 (m, 1H), 4.33-3.96 (m, 3H), 3.38-3.13 (m, 1H), 3.09-2.80 (m, 1H), 2.42-2.36 (m, 6H), 1.60-1.36 (m, 1H), 1.25-0.72 (m, 16H).

### Synthesis of (1R,2S,5S)—N-(1-amino-3-(6-methyl-2-oxo-1,2-dihydroquinolin-3-yl)-1-oxopropan-2-yl)-3-((S)-3,3-dimethyl-2-((5-methylthiazol-2-yl)amino)butanoyl)-6,6-dimethyl-3-azabicyclo[3.1.0]hexane-2-carboxamide (P1.70)

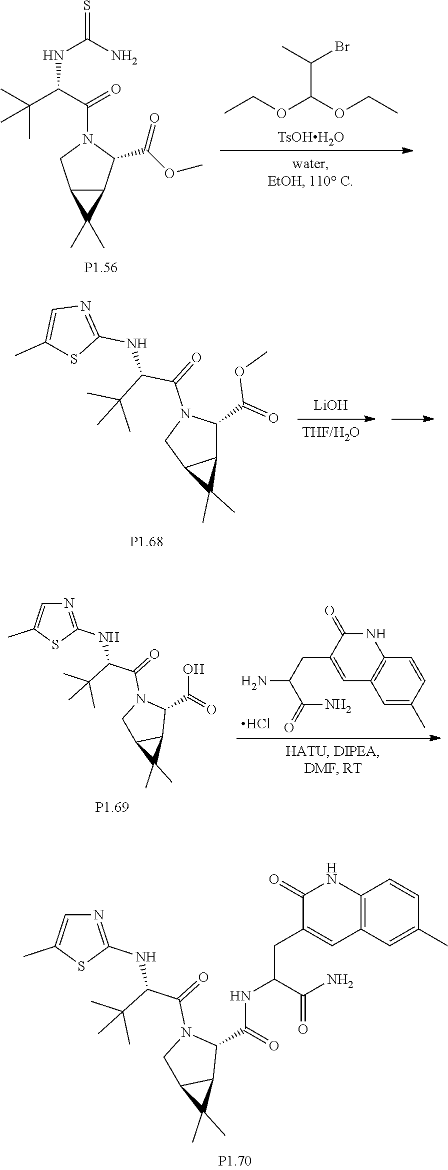

### Preparation 73. Methyl (1R,2S,5S)-3-((S)-3,3-dimethyl-2-((5-methylthiazol-2-yl)amino)butanoyl)-6,6-dimethyl-3-azabicyclo[3.1.0]hexane-2-carboxylate (P1.68)

To a solution of P1.56 (100 mg, 0.292 mmol, 1.0 eq) in ethanol (1.25 mL) and water (0.123 mL) was added 2-bromo-1,1-diethoxypropane (51.75 μL, 0.321 mmol, 1.1 eq) and TsOH·H₂O (2.5 mg, 0.0146 mmol, 0.05 eq) slowly at rt. The reaction mixture was heated to 90° C. for overnight. The reaction mixture was concentrated to get crude product. The residue was purified by silica gel flash column chromatography (3-5% MeOH in DCM) to give P1.68 (60 mg, 54% yield) as a white solid. ¹H NMR (400 MHz, CDCl₃), δ: 6.56 (s, 1H), 5.46 (d, J=10.2 Hz, 1H), 4.42 (s, 1H), 4.36 (d, J=10.2 Hz, 1H), 4.09 (d, J=6.0 Hz, 1H), 3.92-3.88 (m, 1H), 3.72 (s, 1H, 3H), 2.17 (s, 3H), 1.45-1.38 (m, 2H), 1.06 (s, 9H), 1.00 (s, 3H), 0.82 (s, 3H).

### Preparation 74. (1R,2S,5S)-3-((S)-3,3-Dimethyl-2-((5-methylthiazol-2-yl)amino)butanoyl)-6,6-dimethyl-3-azabicyclo[3.1.0]hexane-2-carboxylic acid (P1.69)

A solution of P1.68 (60 mg, 0.158 mmol, 1.0 eq) in THF (0.32 mL) was added to a solution of LiOH (11.4 mg, 0.474 mmol, 3.0 eq) in water (0.32 mL). The resulting mixture was stirred at rt for overnight. The mixture was neutralized with 1N HCl_{(aq)} and extracted with ethyl acetate. The combined organic layers were dried over Na₂SO₄₍ₛ₎, filtered and concentrated in vacuo to give P1.69 (80 mg, 90% yield) as a white solid. ¹H NMR (400 MHz, CD₃OD), δ: 6.59 (s, 1H), 4.41 (s, 1H), 4.26 (s, 1H), 4.10 (d, J=10.8 Hz, 1H), 3.98-3.94 (m, 1H), 2.22 (s, 3H), 1.49-1.45 (m, 2H), 1.09 (s, 9H), 1.03 (s, 3H), 0.83 (s, 3H).

### Preparation 75. (1R,2S,5S)—N-(1-amino-3-(6-methyl-2-oxo-1,2-dihydroquinolin-3-yl)-1-oxopropan-2-yl)-3-((S)-3,3-dimethyl-2-((5-methylthiazol-2-yl)amino)butanoyl)-6,6-dimethyl-3-azabicyclo[3.1.0]hexane-2-carboxamide (P1.70)

To a solution of P1.69 (80 mg, 0.219 mmol, 1.0 eq) in DMF (1.67 mL) was added 2-amino-3-(6-methyl-2-oxo-1,2-dihydroquinolin-3-yl)propanamide (74 mg, 0.263 mmol, 1.2 eq), HATU (91 mg, 0.2406 mmol, 1.1 eq) and diisopropyl ethyl amine (DIPEA, 114.3 μL, 0.656 mmol, 3.0 eq). The resulting mixture was stirred at rt for overnight. The mixture was directly purified by C-18 reversed-phase column chromatography (mobile phase A: water, mobile phase B: methanol, UV: 214 and 254 nm, Flow rate: 35 mL/min, Gradient: 70-80% (% B)) to give P1.70 (70 mg, 54% yield) as a white solid. ¹H NMR (400 MHz, CD₃OD), δ: 7.80 (s, 1H), 7.75 (s, 1H), 7.42-7.21 (m, 6H), 6.59 (s, 1H), 6.57 (s, 1H), 4.87-4.85 (m, 1H), 4.61-4.59 (m, 1H), 4.41 (s, 1H), 4.33 (s, 1H), 4.15-4.00 (m, 5H), 3.94-3.88 (m, 1H), 3.38-3.33 (m, 1H), 3.15-3.05 (m, 2H), 2.85-2.83 (m, 1H), 2.39 (s, 3H), 2.38 (s, 3H), 2.21 (s, 6H), 1.59-1.52 (m, 1H), 1.48-1.41 (m, 2H), 1.22-1.20 (m, 1H), 1.09-0.79 (s, 30H).

### Synthesis of (1R,2S,5S)—N-(1-amino-3-(6-chloro-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.77)

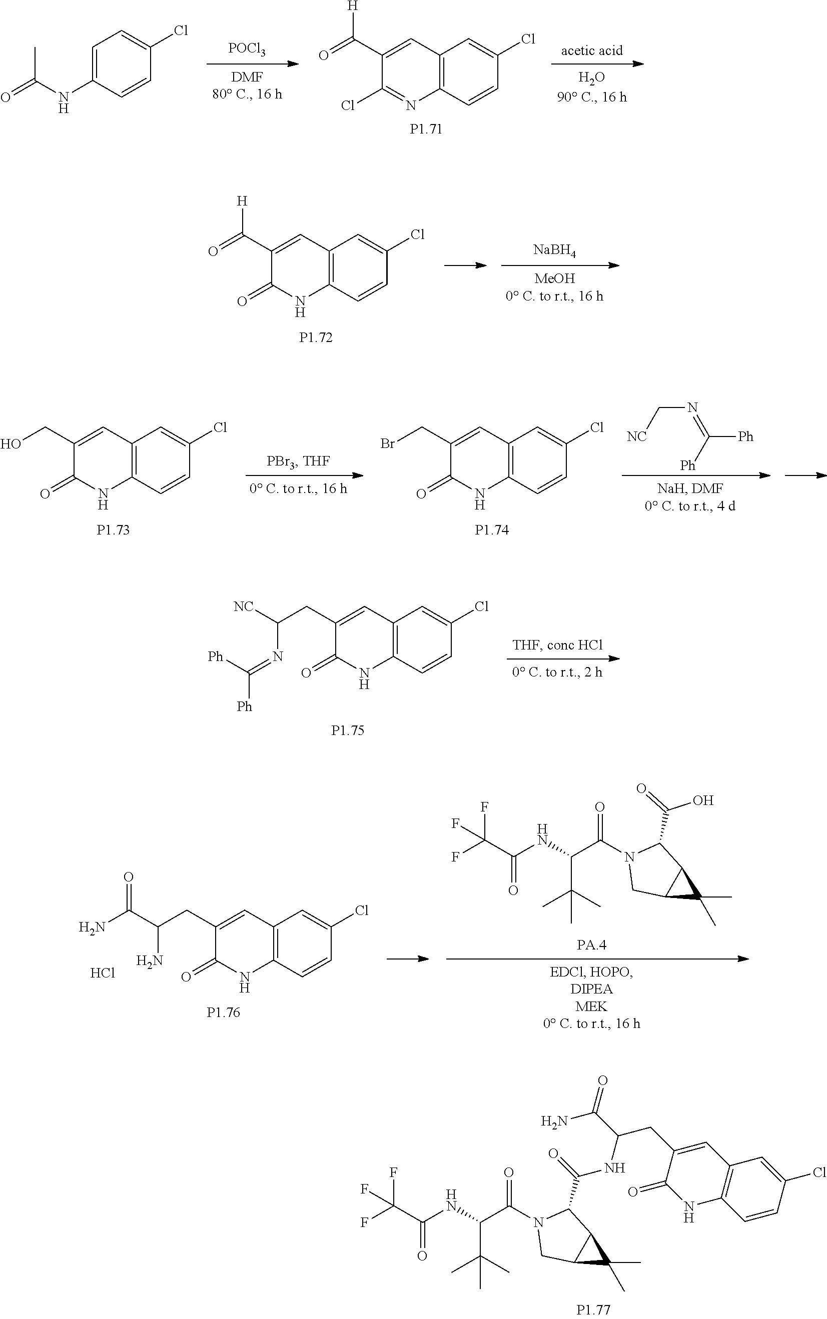

### Preparation 76. 2,6-Dichloroquinoline-3-carbaldehyde (P1.71)

A solution of POCl₃ (150 mL, 1.59 mol, 9.0 eq) in DMF (28 mL) was stirred at 0° C. for 10 min and then at rt for 15 min. To the solution was added N-(4-chlorophenyl)acetamide (30 g, 177.48 mmol, 1.0 eq) at 0° C. in one portion. After the solution was stirred at 90° C. overnight, the reaction mixture was diluted with H₂O (200 mL) and extracted with ethyl acetate (200 mL×3). The organic layers were washed with brine solution, dried over MgSO₄, filtered, and concentrated to give P1.71 as a brown solid (24 g) which was used for the next step without further purification. ¹H NMR (400 MHz, CDCl₃), δ: 10.52 (s, 1H), 8.67 (s, 1H), 8.03-8.00 (m, 1H), 7.96 (d, J=2.4 Hz, 1H), 7.83-7.78 (m, 1H).

### Preparation 77. 6-Chloro-2-oxo-1,2-dihydroquinoline-3-carbaldehyde (P1.72)

A solution of P1.71 (24 g, 106 mmol, 1.0 eq) in a solution of 70% AcOH in H₂O (530 mL) was stirred at 95° C. overnight. The mixture was cooled to rt. The resulting solid was collected by filtration and washed with H₂O. The solid was dissolved in MeOH. The solution was dried over MgSO₄, filtered, and concentrated to give the desired product P1.72 as a pale-yellow solid (10 g) which was directly used for next step without further purification. ¹H NMR (400 MHz, DMSO-d₆), δ: 12.3 (br. s, 1H), 10.2 (s, 1H), 8.48 (s, 1H), 8.06 (d, J=2.4 Hz, 1H), 7.68 (dd, J=8.8, 2.4 Hz, 1H), 7.36 (d, J=8.8 Hz, 1H).

### Preparation 78. 6-Chloro-3-(hydroxymethyl)quinolin-2(1H)-one (P1.73)

A solution of P1.72 (3.0 g, 14.48 mmol, 1.0 eq) in MeOH (96 mL) was stirred at 0° C. for 10 min followed by the addition of NaBH₄ (1.4 g, 36.2 mmol, 2.5 eq) and stirred from 0° C. to rt for overnight. The reaction mixture was carefully diluted with H₂O (100 mL) and extracted with ethyl acetate (200 mL×4). The organic layers were washed with brine solution, dried over MgSO₄, filtered, and concentrated. The residue was purified by silica gel chromatography (40-50% ethyl acetate in hexane) to give P1.73 (430 mg). ¹H NMR (400 MHz, DMSO-d₆), δ: 11.91 (br. s, 1H), 7.85-7.83 (m, 2H), 7.48 (dd, J=8.8, 2.4 Hz, 1H), 7.30 (d, J=8.8 Hz, 1H), 5.28 (t, J=5.6 Hz, OH), 4.39 (d, J=5.6 Hz, 2H).

### Preparation 79. 3-(Bromomethyl)-6-chloroquinolin-2(1H)-one (P1.74)

A solution of P1.73 (430 mg, 1.59 mmol, 1 eq) in THF (8.0 mL) was stirred at 0° C. for 10 min followed by addition of PBr₃ (0.30 mL, 3.18 mmol, 2 eq) in one portion and stirred at rt for overnight. The reaction mixture was cooled to 0° C., carefully diluted with H₂O, and extracted with ethyl acetate (100 mL×3). The organic layers were washed with brine solution, dried over MgSO₄, filtered, and concentrated to give P1.74 (960 mg) which was directly used for the next step without further purification. ¹H NMR (400 MHz, DMSO-d₆), δ: 12.11 (br. s, 1H), 8.12 (s, 1H), 7.81 (d, J=2.4 Hz, 1H), 7.56 (dd, J=8.8, 2.4 Hz, 1H), 7.32 (d, J=8.8 Hz, 1H), 4.55 (s, 2H); LCMS (ESI) m/z calc. for C₁₀H₇BrClNO 270.94. found, 271.9 [M+H]⁺.

### Preparation 80. 3-(6-Chloro-2-oxo-1,2-dihydroquinolin-3-yl)-2 ((diphenylmethylene)amino)propanenitrile (P1.75)

A solution of 2-((diphenylmethylene)amino)acetonitrile (956 mg, 4.34 mmol, 1.3 eq) in DMF (8.0 mL) was cooled at 0° C. for 10 min followed by the addition of NaH (175 mg, 4.34 mmol, 1.3 eq) in one portion and stirred for 5 min. P1.74 (900 mg, 3.34 mmol, 1.0 eq) was dissolved in DMF (8.0 mL) and dropwise added to the reaction mixture. The mixture was stirred for 4 h, based upon the monitoring by TLC. The reaction mixture was quenched with cold water and extracted with ethyl acetate (100 mL×3). The organic layers were washed with brine solution, dried over MgSO₄, filtered, and concentrated. The residue was purified by silica gel chromatography (30% ethyl acetate in n-hexane) to give P1.75 (600 mg) as a yellow oil. ¹H NMR (400 MHz, CDCl₃), δ: 10.46 (br. s, 1H), 7.74 (s, 1H), 7.65-7.59 (m, 2H), 7.55-7.49 (m, 2H), 7.37-7.30 (m, 6H), 7.23-7.19 (m, 1H), 7.07-7.05 (m, 2H), 4.86-4.83 (m, 1H), 3.36-3.18 (m, 2H).

### Preparation 81. 2-Amino-3-(6-chloro-2-oxo-1,2-dihydroquinolin-3-yl)propanamide (P1.76)

A solution of P1.75 (600 mg, 1.45 mmol, 1.0 eq) in THF (7.2 mL) was cooled to 0° C. for 10 min followed by the addition of conc HCl (0.25 ml, 7.25 mmol, 5 eq) and stirred at rt for 2 h monitored using TLC. The solvent was removed under reduced pressure. The residue was diluted with H₂O and extracted with ethyl acetate (50 ml×2). The aqueous layer was concentrated to give pale yellow solid, then washed with cold H₂O and dissolved in MeOH.

The solution was dried over MgSO₄, filtered, and concentrated to give the desired product (140 mg). ¹H NMR (400 MHz, DMSO-d₆), δ: 12.14 (br. s, 1H), 8.37 (br. s, 1H), 8.25 (br. s, 2H), 7.94 (br. s, 1H), 7.84-7.76 (m, 1H), 7.56-7.52 (m, 1H), 7.36 (dd, J=8.8, 2.4 Hz, 1H), 4.08-4.03 (m, 1H), 3.14-2.91 (m, 2H); LCMS (ESI) m/z calc. for C₁₂H₁₂C₁N₃O₂ 265.06. found, 266.1 [M+H]⁺.

### Preparation 82. (1R,2S,5S)—N-(1-Amino-3-(6-chloro-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.77)

To a solution of P1.76 (102.9 mg, 0.362 mmol, 1.1 eq), PA.4 (120.0 mg, 0.329 mmol, 1.0 eq) in butan-2-one (MEK, 1.1 mL) was added 2-hydroxypyridine 1-oxide (HOPO, 9.2 mg, 0.082 mmol, 0.25 eq) and 1-(3-dimethylaminopropyl)-3-ethylcarbodiimide hydrochloride (EDCI, 82.1 mg, 0.428 mmol, 1.3 eq) at 0° C. N,N-Diisopropylethylamine (DIEA, 0.23 mL, 1.32 mmol, 4.0 eq) was added slowly to the reaction mixture and the resulting mixture was stirred at rt for 16 h. The solution was diluted with a solution of EtOAc/MTBE (1/1) and washed with 1 N HCl_{(aq)}, brine, saturated NaHCO_{3(aq)}, and brine. The organic layer was dried over Na₂SO₄₍ₛ₎, filtered, and then concentrated under reduced pressure. The residue was purified by C-18 reversed-phase column chromatography (mobile phase A: water (with 0.1% NH₄HCO₃), mobile phase B: acetonitrile, UV: 214 and 254 nm, Flow rate: 35 mL/min, Gradient: 3-90% (% B)) to give P1.77 (73 mg, 36% yield) as a white solid. ¹H NMR (400 MHz, DMSO-d₆), δ: 11.95-11.88 (m, 2H), 9.42-9.23 (m, 2H), 8.47 (d, J=8.8 Hz, 1H), 8.23 (d, J=7.2 Hz, 1H), 7.68-7.57 (m, 4H), 7.47-7.41 (m, 2H), 7.27-7.21 (m, 3H), 7.07 (s, 2H), 6.94 (s, 1H), 4.51-4.27 (m, 4H), 4.10 (s, 1H), 4.0 (s, 1H), 3.95-3.89 (m, 1H), 3.78-3.71 (m, 1H), 3.60-3.54 (m, 2H), 3.17-3.11 (m, 1H), 2.97-2.89 (m, 1H), 2.83-2.75 (m, 1H), 2.57-2.49 (m, 1H), 1.48-1.42 (m, 1H), 1.27-1.21 (m, 1H), 1.09 (d, J=8.0 Hz, 1H), 0.99-0.86 (m, 19H), 0.84-0.71 (m, 12H).

### Synthesis of (1R,2S,5S)—N-(1-amino-3-(7-chloro-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.84)

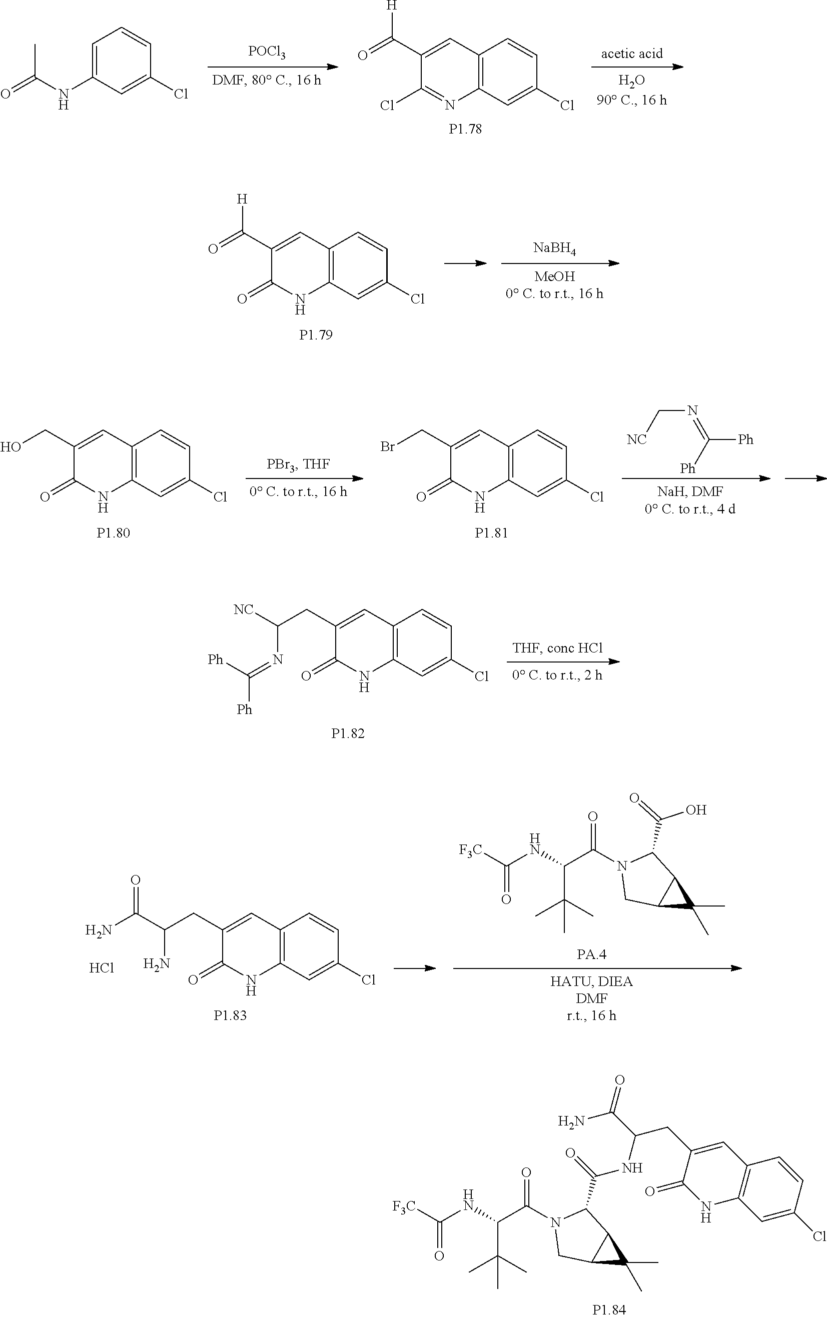

### Preparation 83. 2,7-Dichloroquinoline-3-carbaldehyde (P1.78)

A solution of POCl₃ (25 mL, 266 mmol, 9 eq) in DMF (5 mL) was stirred at 0° C. for 10 min and then stirred at rt for 15 min. To the reaction mixture was added N-(3-chlorophenyl)acetamide (5.0 g, 29.6 mmol, 1.0 eq) at 0° C. in one portion and stirred at 90° C. overnight. The reaction mixture was diluted with H₂O (200 mL) and extracted with ethyl acetate (200 mL×3). The organic layers were washed with brine solution, dried over MgSO₄, filtered, and concentrated to give P1.78 (5.86 g) as a brown solid which was directly used for next step without further purification.

### Preparation 84. 6-Chloro-2-oxo-1,2-dihydroquinoline-3-carbaldehyde (P1.79)

A solution of P1.78 (5.86 g, 25.9 mmol, 1.0 eq) in a solution of 70% AcOH in H₂O (260 mL) was stirred at 95° C. overnight. The mixture was cooled to rt. The resulting solid was collected by filtration and washed with excess H₂O. The solid was dissolved in MeOH. The solution was dried over MgSO₄, filtered, and concentrated to give the desired product P1.79 (3.01 g) as a pale-yellow solid which was directly used for the next step without further purification. ¹H NMR (400 MHz, DMSO-d₆), δ: 12.28 (br. s, 1H), 10.21 (s, 1H), 8.52 (s, 1H), 7.97 (d, J=8.8 Hz, 1H), 7.38-7.30 (m, 2H).

### Preparation 85. 7-Chloro-3-(hydroxymethyl)quinolin-2(1H)-one (P1.80)

A solution of P1.79 (2.0 g, 9.7 mmol, 1.0 eq) in MeOH (64 mL, 0.15M) was stirred at 0° C. for 10 min followed by the addition of NaBH₄ (903 mg, 24.17 mmol, 2.5 eq) and stirred from 0° C. to rt for overnight. The reaction mixture was carefully diluted with H₂O (80 mL) and extracted with ethyl acetate (150 mL×4). The organic layers were washed with brine solution, dried over MgSO₄, filtered, and concentrated. The crude was purified by silica gel chromatography (40-50% ethyl acetate in hexane) to give P1.80 (1.1 g). ¹H NMR (400 MHz, DMSO-d₆), δ: 11.87 (br. s, 1H), 7.86 (s, 1H), 7.73 (d, J=8.6 Hz, 1H), 7.32 (d, J=2.0 Hz, 1H), 7.20 (dd, J=8.6, 2.0 Hz, 1H), 5.27 (t, J=5.2 Hz, 1H), 4.38 (d, J=5.2 Hz, 2H).

### Preparation 86. 3-(Bromomethyl)-7-chloroquinolin-2(1H)-one (P1.81)

A solution of P1.80 (1.1 g, 4.07 mmol, 1.0 eq) in THF (20 mL) was stirred at 0° C. for 10 min followed by addition of PBr₃ (0.78 ml, 8.14 mmol, 2.0 eq) in one portion and stirred at rt for overnight. The reaction mixture was cooled to 0° C., carefully diluted with H₂O, and extracted with ethyl acetate (100 mL×3). The organic layers were washed with brine solution dried over MgSO₄, filtered, and concentrated. The crude was purified by silica gel chromatography (30-35% ethyl acetate in n-hexane) to give P1.81 (400 mg). ¹H NMR (400 MHz, DMSO-d₆), δ: 12.05 (br. s, 1H), 8.17 (s, 1H), 7.71 (d, J=8.4 Hz, 1H), 7.33 (d, J=2.0 Hz, 1H) 7.24 (dd, J=8.4, 2.0 Hz, 1H), 4.56 (s, 2H).

### Preparation 87. 3-(7-Chloro-2-oxo-1,2-dihydroquinolin-3-yl)-2-((diphenylmethylene)amino)propanenitrile (P1.82)

A solution of 2-((diphenylmethylene)amino)acetonitrile (375 mg, 1.70 mmol, 1.3 eq) in DMF (3.0 mL) was cooled at 0° C. for 10 min followed by the addition of NaH (70 mg, 1.70 mmol, 1.3 eq) in one portion and stirred for 5 min. P1.81 (400 mg, 1.30 mmol, 1.0 eq) was dissolved in DMF (4 mL) and dropwise added to the reaction mixture. The mixture was stirred for 4 h, based upon the monitoring by TLC. The reaction mixture was quenched with cold H₂O and extracted with ethyl acetate (100 mL×3). The organic layers were washed with brine solution, dried over MgSO₄, filtered, and concentrated. The crude was purified by silica gel chromatography (30% ethyl acetate in n-hexane) to give P1.82 (385 mg) as a pale-yellow oil. ¹H NMR (400 MHz, DMSO-d₆): δ 11.87 (br. s, 1H), 7.90 (s, 1H), 7.64 (d, J=8.8 Hz, 1H), 7.52-7.44 (m, 6H), 7.42-7.35 (m, 2H), 7.29 (d, J=2.4 Hz, 1H), 7.19 (dd, J=8.4, 2.0 Hz, 1H), 6.98-6.95 (m, 2H), 4.73-4.68 (m, 1H), 3.19-3.01 (m, 1H).

### Preparation 88. 2-Amino-3-(7-chloro-2-oxo-1,2-dihydroquinolin-3-yl)propanamide (P1.83)

A solution of P1.82 (385 mg, 0.93 mmol, 1.0 eq) in THF (5 mL) was cooled to 0° C. for 10 min followed by the addition of concentrated HCl solution (0.14 ml, 4.65 mmol, 5 eq) and stirred at rt for 2 h, monitored using TLC. The solution was concentrated to give a residue. The residue was diluted with H₂O and extracted with ethyl acetate (50 mL×2). The aqueous layer was concentrated to give pale yellow solid, which was washed with cold H₂O and dissolved in MeOH. The solution was dried over MgSO₄, filtered, and concentrated to give P1.83 (328 mg). ¹H NMR (400 MHz, DMSO-d₆), δ: 12.10 (br. s, 1H), 8.27 (br. s, 2H), 7.95 (br. s, 2H), 7.84 (s, 1H), 7.67 (d, J=8.4 Hz, 1H), 7.54 (br. s, 1H), 7.40-7.37 (m, 1H), 7.23 (dd, J=8.4, 2.0 Hz, 1H), 4.05-4.03 (m, 1H), 3.04-2.90 (m, 2H); LCMS (ESI) m/z calc. for C₁₂H₁₂C₁N₃O₂ 265.06. found, 266.1 [M+H]⁺.

### Preparation 89. (1R,2S,5S)—N-(1-Amino-3-(7-chloro-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.84)

To a solution of PA.4 (100.0 mg, 0.274 mmol, 1.0 eq) in DMF (2.0 mL) was added P1.83 (99.5 mg, 0.329 mmol, 1.2 eq), HATU (114.8 mg, 0.302 mmol, 1.1 eq) and DIEA (0.14 mL, 0.823 mmol, 3.0 eq). The reaction mixture was stirred at rt for 16 h. The mixture was concentrated and then purified by C-18 reversed-phase column chromatography (mobile phase A: water (with 0.1% NH₄HCO₃), mobile phase B: acetonitrile, UV: 214 and 254 nm, Flow rate: 40 mL/min, Gradient: 3-90% (% B)) to give P1.84 (90 mg, 54% yield) as a white solid. ¹H NMR (400 MHz, DMSO-d₆), δ: 11.94 (br. s, 1H), 11.88 (br. s, 1H), 9.41-9.38 (m, 2H), 8.50 (d, J=8.8 Hz, 1H), 8.28 (d, J=7.6 Hz, 4H), 7.77-7.57 (m, 4H), 7.35-6.95 (m, 8H), 4.55-4.31 (m, 4H), 4.14 (s, 1H), 4.11 (s, 1H), 3.98-3.90 (m, 1H), 3.83-3.76 (m, 1H), 3.63-3.62 (m, 2H), 3.18-3.11 (m, 1H), 2.98-2.90 (m, 1H), 2.86-2.78 (m, 1H), 2.63-2.55 (m, 1H), 1.51-1.44 (m, 1H), 1.35-1.30 (m, 1H), 1.16 (d, J=8.0 Hz, 1H), 1.00-0.90 (m, 19H), 0.88-0.78 (m, 12H).

### Synthesis of (1R,2S,5S)—N-(1-amino-3-(1-methyl-2-oxo-1,2-dihydropyridin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.90)

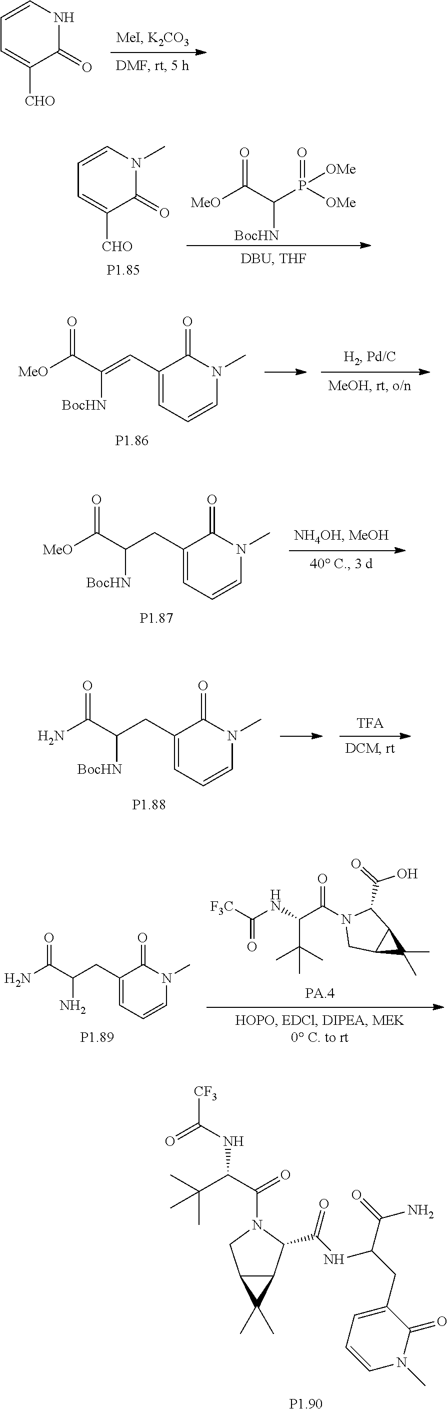

### Preparation 90. Methyl (Z)-2-((tert-butoxycarbonyl)amino)-3-(1-methyl-2-oxo-1,2-dihydropyridin-3-yl)acrylate (P1.85)

To a solution of 2-oxo-1H-pyridine-3-carbaldehyde (7.86 g, 63.85 mmol) in DMF (120 mL) was added potassium carbonate (26.47 g, 191.5 mmol) and Mel (4.17 mL), and the resulting mixture was stirred for overnight at rt. The mixture was filtered through a celite pad and the filtrate was concentrated under reduced pressure. The residue was diluted with water and extracted with EtOAc. The combined organic layers were dried over Na₂SO₄, filtered, and concentrated to give P1.85 (2.85 g, 33%) as a yellow solid. ¹H NMR (400 MHz, CDCl₃), δ: 10.35 (s, 1H), 8.04 (dd, J=7.2, 2.2 Hz, 1H), 7.62 (d, J=2.2 Hz, 1H), 6.34-6.30 (m, 1H), 3.63 (s, 3H).

### Preparation 91. Methyl (Z)-2-((tert-butoxycarbonyl)amino)-3-(1-methyl-2-oxo-1,2-dihydropyridin-3-yl)acrylate (P1.86)

To a solution of P1.85 (3.28 g, 23.9 mmol) in anh. THF (120 mL) was added a solution of methyl 2-((tert-butoxycarbonyl)amino)-2-(dimethoxyphosphoryl)acetate (9.20 g, 30.95 mmol) and DBU (4.64 mL) in anh. THF (120 mL) in ice bath. The resulting mixture was stirred for overnight at rt. The mixture was concentrated under reduced pressure. The residue was purified by silica gel chromatography (0-70% EtOAc in n-hexane) to give P1.86 (5.2 g, 71%) as a solid. ¹H NMR (400 MHz, CDCl₃), δ: 8.54 (br. s, 2H), 7.51-7.49 (m, 2H), 7.38-7.36 (m, 2H), 6.68 (s, 2H), 6.30-6.24 (m, 2H), 3.84-3.83 (m, 6H), 3.63 (s, 6H), 1.45-1.44 (m, 18H).

### Preparation 92. Methyl 2-((tert-butoxycarbonyl)amino)-3-(1-methyl-2-oxo-1,2-dihydropyridin-3-yl)propanoate (P1.87)

A solution of P1.86 (1.11 g, 3.60 mmol) and 10% Pd on carbon (110 mg) in MeOH (20 mL) was stirred for overnight at rt under hydrogen (1 atm). The mixture was filtered through a pad of celite, and the filtrate was concentrated under reduced pressure to give P1.87 (1.2 g, crude). ¹H NMR (400 MHz, CDCl₃), δ: 7.22-7.19 (m, 2H), 6.29 (br. s, 1H), 6.11 (dd, J=6.8, 6.8 Hz, 1H), 4.50-4.45 (m, 1H), 3.70 (s, 3H), 3.56 (s, 3H), 3.07-3.03 (m, 1H), 2.97-2.91 (m, 1H), 1.45 (s, 9H).

### Preparation 93. tert-Butyl (1-amino-3-(1-methyl-2-oxo-1,2-dihydropyridin-3-yl)-1-oxopropan-2-yl)carbamate (P1.88)

To a solution of P1.87 (crude, 16.90 mmol) in MeOH (54.0 mL) was added ammonium hydroxide solution (27 mL). The reaction was stirred at rt for 3 days and concentrated to remove solvent. The crude was purified by silica gel chromatography (0-10% MeOH in DCM) to give P1.88 (2.8 g, 56% over 2 steps) as a white solid. ¹H NMR (400 MHz, CDCl₃), δ: 7.35-7.32 (m, 1H), 7.24-7.23 (m, 1H), 6.82 (br. s, 1H), 6.60 (br. s, 1H), 6.17 (dd, J=6.8, 6.8 Hz, 1H), 5.36 (br. s, 1H), 4.33 (br. s, 1H), 3.59 (s, 3H), 3.10-3.05 (m, 1H), 2.89-2.84 (m, 1H), 1.405 (s, 9H).

### Preparation 94. 2-Amino-3-(1-methyl-2-oxo-1,2-dihydropyridin-3-yl)propanamide (P1.89)

To a solution of P1-88 (300 mg, 1.01 mmol) in DCM (4.0 mL) was added TFA (2.0 mL). The reaction was stirred at rt for overnight and concentrated to remove solvent. The crude was purified by C18 reverse phase column chromatography (0-20% MeOH in water) to give P1.89 (194 mg, 99%) as a yellow oil. ¹H NMR (400 MHz, CD₃OD), δ: 7.64-7.62 (m, 1H), 7.47-7.45 (m, 1H), 6.35 (dd, J=6.8, 6.8 Hz, 1H), 4.17-4.14 (m, 1H), 3.58 (s, 3H), 3.15-3.10 (m, 1H), 2.97-2.92 (m, 1H); LCMS (ESI) m/z calc. for C₉H₁₃N₃O₂ 195.10. found, 196.0 [M+H]⁺.

### Preparation 95. (1R,2S,5S)—N-(1-amino-3-(1-methyl-2-oxo-1,2-dihydropyridin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.90)

To a solution of P1.89 (270 mg, 1.38 mmol), I-8 (271 mg, 0.74 mmol) in butan-2-one (3.0 mL) was added 2-hydroxypyridine 1-oxide (21 mg, 0.19 mmol) and 1-(3-dimethylaminopropyl)-3-ethylcarbodiimide hydrochloride (183 mg, 0.96 mmol) at 0° C. After N,N-diisopropylethylamine (0.51 mL, 2.96 mmol) was added slowly to the reaction mixture, the resulting mixture was stirred for overnight. It was concentrated to remove solvent. The residue was purified by reverse-phase chromatography (0-65% MeOH in water) to give P1.90 (525 mg, 57%) as a white solid. ¹H NMR (400 MHz, CDCl₃), δ: 9.25-9.24 (m, 1H), 8.32-8.31 (m, 1H), 7.43-7.39 (m, 2H), 7.09-7.05 (m, 2H), 6.87-6.85 (m, 2H), 6.24-6.19 (m, 2H), 5.30 (br. s, 1H), 5.14 (br. s, 1H), 4.62-4.58 (m, 2H), 4.52-4.50 (m, 2H), 4.29-4.25 (m, 1H), 4.22 (s, 1H), 4.16 (s, 1H), 4.07-4.03 (m, 1H), 3.84-3.77 (m, 2H), 3.62 (s, 3H), 3.59 (s, 3H), 3.12-3.10 (m, 2H), 3.03-3.00 (m, 1H), 2.83-2.82 (m, 1H), 1.53-1.50 (m, 2H), 1.44-1.42 (m, 2H), 1.07-1.04 (m, 12H), 1.00 (s, 12H), 0.96 (s, 3H), 0.86 (s, 3H); LCMS (ESI) m/z calc. for C₂₅H₃₄F₃N₅O₅ 541.25. found, 542.3 [M+H]⁺.

### Synthesis of (1R,2S,5S)—N-(1-amino-i-oxo-3-(2-oxo-1,2,5,6,7,8-hexahydroquinolin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.99)

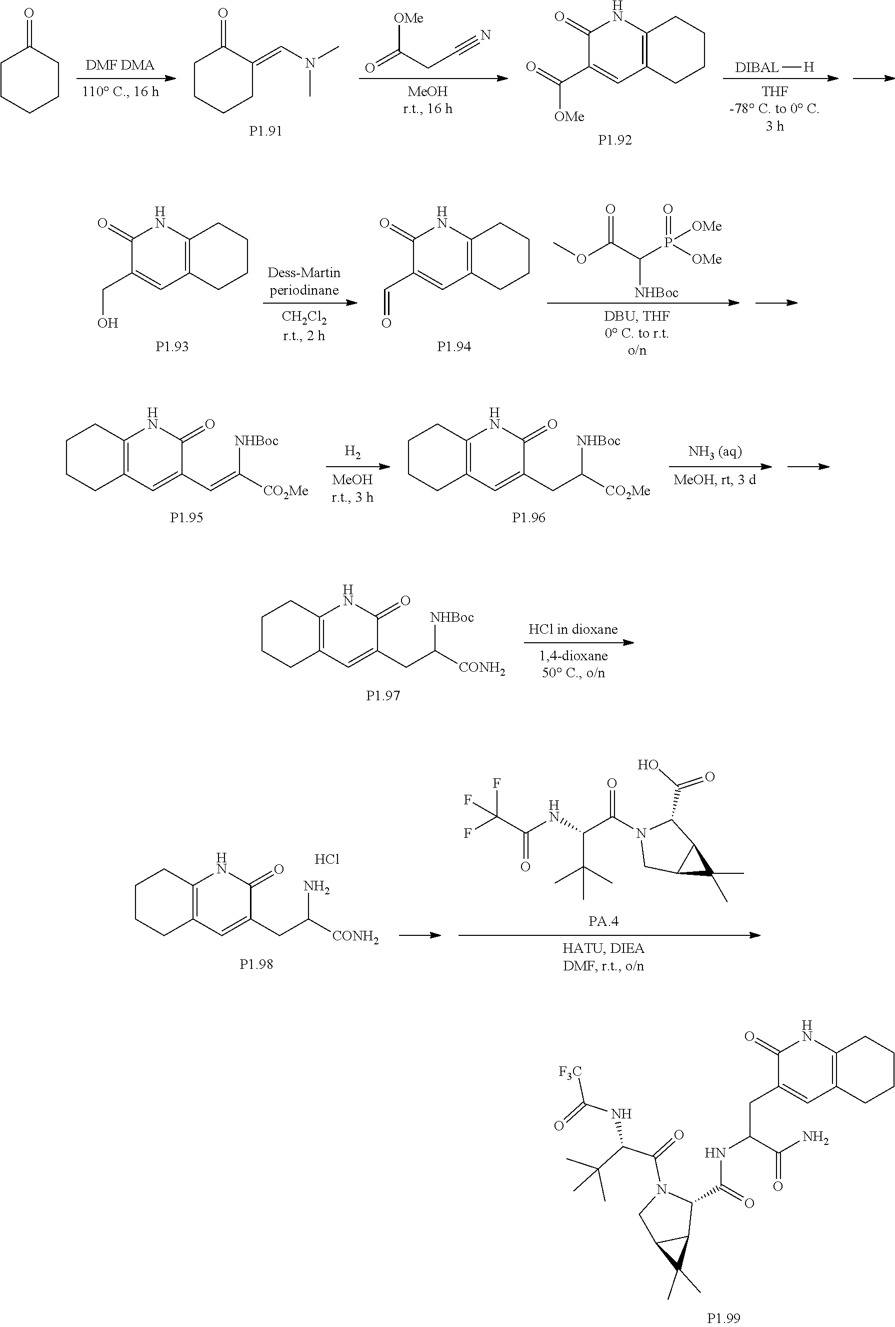

### Preparation 96. (E)-2-((Dimethylamino)methylene)cyclohexan-1-one (P1.91)

A solution of cyclohexanone (5.00 g, 50.9 mmol) in N,N-dimethylformamide dimethyl acetal (6.50 mL, 50.9 mmol) was stirred at 110° C. for 16 h. The mixture was concentrated under reduced pressure to give P1.91 (4.5 g, crude) as a brown oil which was used in next step without further purification. ¹H NMR (400 MHz, CDCl₃), δ: 7.47 (s, 1H), 3.05 (s, 6H), 2.66-2.63 (m, 2H), 2.32-2.28 (m, 2H), 1.72-1.68 (m, 2H), 1.65-1.60 (m, 2H).

### Preparation 97. Methyl 2-oxo-1,2,5,6,7,8-hexahydroquinoline-3-carboxylate (P1.92)

A solution of P1.91 (4 g, crude, 45.84 mmol) and methyl cyanoacetate in methanol (23 mL) was stirred at rt for 16 h. The solvent was removed under reduced pressure. The residue was purified by silica gel column chromatography (0-7% methanol in DCM) to give P1.92 (3.12 g, 30% over 2 steps) as a purple solid. ¹H NMR (400 MHz, CDCl₃), δ: 8.01 (s, 1H), 3.89 (s, 3H), 2.77 (t, J=6.0 Hz, 2H), 2.55 (t, J=6.0 Hz, 2H), 1.91-1.69 (m, 4H).

### Preparation 98. 3-(Hydroxymethyl)-5,6,7,8-tetrahydroquinolin-2(1H)-one (P1.93)

To a solution of P1.92 (500 mg, 2.42 mmol) in anh. THF was added diisobutylaluminium hydride at −78° C. slowly, then stirred at −78° C. for 15 min. After the mixture was stirred at 0° C. for 3 h. The mixture was treated with NaHCO_{3(aq)} and extracted with EtOAc. The combined organic layers were dried over Na₂SO₄, filtered, and concentrated under reduced pressure to give P1.93 (250 mg, 57%) as a yellow solid which was used without further purification. ¹H NMR (400 MHz, CDCl₃), δ: 12.09 (s, 1H), 7.12 (s, 1H), 4.55 (s, 2H), 3.93 (br. s, 1H), 2.62 (t, J=6.0 Hz, 2H), 2.48 (t, J=6.0 Hz, 2H), 1.85-1.70 (m, 4H).

### Preparation 99. 2-Oxo-1,2,5,6,7,8-hexahydroquinoline-3-carbaldehyde (P1.94)

To a solution of P1.93 (970 mg, 5.41 mmol, 1.0 eq) in DCM (54 mL) was added Dess-Martin periodinane (2.41 g, 5.68 mmol, 1.05 eq), and it was stirred at rt for 2 h. To the reaction mixture added 1 M Na₂S₂O_{3(aq)} (8.1 mL, 8.11 mmol, 1.5 eq) and then stirred at rt for another 1 h. The mixture was filtered and the filtrate was concentrated. The residue was purified by silica gel column chromatography (0-10% methanol in DCM) to give P1.94 (730 mg, 76%) as a yellow solid. ¹H NMR (400 MHz, CDCl₃), δ: 10.27 (s, 1H), 7.90 (s, 1H), 2.75 (t, J=6.4 Hz, 2H), 2.57 (t, J=6.4 Hz, 2H), 1.86-1.79 (m, 4H).

### Preparation 100. (Z)-2-((tert-Butoxycarbonyl)amino)-3-(2-oxo-1,2,5,6,7,8-hexahydroquinolin-3-yl)acrylate (P1.95)

To a solution of (±)-Boc-α-phosphonoglycine trimethyl ester (1.24 g, 4.12 mmol, 1.0 eq) in anh. THF (25 mL) was added DBU (0.88 mL, 5.86 mmol, 1.3 eq) slowly at 0° C. After the reaction was stirred at 0° C. for 30 min, the ylide solution was added slowly into a solution of P1.94 (730 mg, 4.12 mmol, 1.0 eq) in anh. THF (25 mL) at 0° C. The mixture was stirred at rt overnight. The solvent was removed under reduced pressure. The residue was purified by silica gel column chromatography (0-10% methanol in dichloromethane) to give P1.95 (730 mg, 51% yield) as a solid. ¹H NMR (400 MHz, CDCl₃), δ: 12.72 (br. s, 1H), 9.21 (s, 1H), 7.34 (s, 1H), 6.57 (s, 1H), 3.85 (s, 3H), 2.82-2.79 (m, 2H), 2.53-2.51 (m, 2H), 1.84-1.70 (m, 4H), 1.42 (s, 9H).

### Preparation 101. Methyl 2-((tert-butoxycarbonyl)amino)-3-(2-oxo-1,2,5,6,7,8-hexahydroquinolin-3-yl)propanoate (P1.96)

To a solution of P1.95 (730 mg, 2.10 mmol, 1.0 eq) in methanol (50 mL) was added Pd/C powder (700 mg, 100% wt). The reaction was stirred at rt for 3 h under hydrogen balloon (1 atm). The solution was filtered through a pad of celite. The filtrate was concentrated under reduced pressure. The residue was purified by silica gel column chromatography (0-10% methanol in DCM) to give P1.96 (420 mg, 57% yield) as a white solid. ¹H NMR (400 MHz, CDCl₃), δ: 12.31 (br. s, 1H), 7.07 (s, 1H), 6.83 (d, J=6.0 Hz, 1H), 4.40-4.36 (m, 1H), 3.70 (s, 3H), 2.95-2.93 (m, 2H), 2.69-2.66 (m, 2H), 2.46-2.43 (m, 2H), 1.82-1.65 (m, 4H), 1.40 (s, 9H).

### Preparation 102. tert-Butyl (1-amino-1-oxo-3-(2-oxo-1,2,5,6,7,8-hexahydroquinolin-3-yl)propan-2-yl)carbamate (P1.97)

To a solution of P1.96 (320 mg, 0.91 mmol, 1.0 eq) in methanol (10 mL) added NH₄OH_{(aq)} (5.0 mL). The reaction was stirred at rt for 3 days. The mixture was concentrated to remove solvent to give crude P1.97 (305 mg, 99% yield) as a white solid. ¹H NMR (400 MHz, DMSO-d₆), δ: 11.40 (br. s, 1H), 7.16 (s, 1H), 7.04-7.00 (m, 2H), 6.91 (s, 1H), 4.01-3.96 (m, 1H), 2.74-2.66 (m, 1H), 2.44-2.41 (m, 2H), 2.34-2.31 (m, 1H), 1.64-1.62 (m, 4H), 1.31 (s, 9H).

### Preparation 103. 2-Amino-3-(2-oxo-1,2,5,6,7,8-hexahydroquinolin-3-yl)propanamide hydrochloride (P1.98)

To a mixture of P1.97 (70 mg, 0.21 mmol, 1.0 eq) in 1,4-dioxane (1.5 mL) was added 4 M HCl in 1,4-dioxane (0.26 mL, 1.05 mmol, 5.0 eq). The reaction was stirred at 50° C. for overnight. The solvent was removed under reduced pressure to give P1.98 (76 mg, crude) as a white solid which was used in the next step without further purification. ¹H NMR (400 MHz, DMSO-d₆), δ: 11.76 (br. s, 1H), 8.38 (s, 3H), 7.89 (s, 1H), 7.49 (s, 1H), 7.14 (s, 1H), 3.97-3.95 (m, 1H), 2.91-2.80 (m, 2H), 2.48-2.46 (m, 2H), 2.37 (br. s, 2H), 1.68-1.64 (m, 4H).

### Preparation 104. (1R,2S,5S)—N-(1-amino-1-oxo-3-(2-oxo-1,2,5,6,7,8-hexahydroquinolin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.99)

To a solution of PA.4 (64 mg, 0.18 mmol, 1.0 eq), P1.98 (76 mg, 0.21 mmol, 1.2 eq), and DIPEA (0.091 mL, 0.54 mmol, 3.0 eq) in DMF (1.2 mL) was added HATU (73 mg, 0.20 mmol, 1.1 eq) at 0° C. The reaction mixture was stirred at rt for overnight. The solution was purified by C18 column (5-100% MeOH in H₂O containing 0.1% NH₄HCO₃) to give P1.99 (46 mg, 45%) as a white solid. ¹H NMR (400 MHz, CD₃OD), δ: 7.26 (s, 1H), 7.21 (s, 1H), 4.61-4.44 (m, 4H), 4.23-3.99 (m, 4H), 3.83-3.73 (m, 4H), 3.18-2.74 (m, 4H), 2.56-2.48 (m, 8H), 1.79-1.73 (m, 8H), 1.61-1.56 (m, 2H), 1.43-1.20 (m, 2H), 1.06-0.89 (m, 30H). P1.99 contains two diastereoisomers.

### Synthesis of (1R,2S,5S)—N-(1-amino-i-oxo-3-(2-oxo-1,2,5,6,7,8-hexahydroquinolin-3-yl)propan-2-yl)-3-((S)-3,3-dimethyl-2-(methylsulfonamido)butanoyl)-6,6-dimethyl-3-azabicyclo[3.1.0]hexane-2-carboxamide (P1.100)

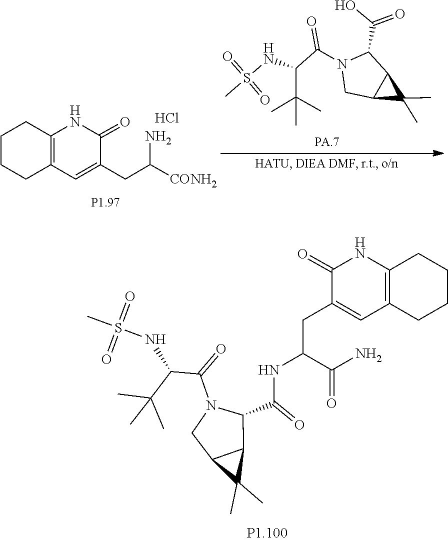

### Preparation 105. (1R,2S,5S)—N-(1-amino-1-oxo-3-(2-oxo-1,2,5,6,7,8-hexahydroquinolin-3-yl)propan-2-yl)-3-((S)-3,3-dimethyl-2-(methylsulfonamido)butanoyl)-6,6-dimethyl-3-azabicyclo[3.1.0]hexane-2-carboxamide (P1.100)

To a solution of PA.7 (166 mg, 0.29 mmol, 1.0 eq) in DMF (2 mL) was added P1.97 (94 mg, 0.35 mmol, 1.2 eq), HATU (120 mg, 0.32 mmol, 1.1 eq) and DIPEA (0.15 mL, 0.87 mmol, 3.0 eq). The reaction mixture was stirred at rt for overnight. The mixture was concentrated and purified by C-18 reversed-phase column chromatography (5-100% MeOH in H₂O containing 0.1% NH₄HCO₃) to give P1.100 (103 mg, 64% yield) as a white solid. ¹H NMR (400 MHz, CD₃OD), δ: 7.27 (s, 1H), 7.21 (s, 1H), 4.61-4.45 (m, 2H), 4.25-4.24 (m, 2H), 4.05-3.79 (m, 6H), 3.14-2.77 (m, 10H), 2.56-2.48 (m, 8H), 1.78-1.75 (m, 8H), 1.62-1.58 (m, 2H), 1.42-1.82 (m, 2H), 1.07-0.94 (m, 30H).

### Synthesis of 2-amino-3-(2-oxo-1,5,7,8-tetrahydro-2H-pyrano[4,3-b]pyridin-3-yl)propanamide (P1.108)

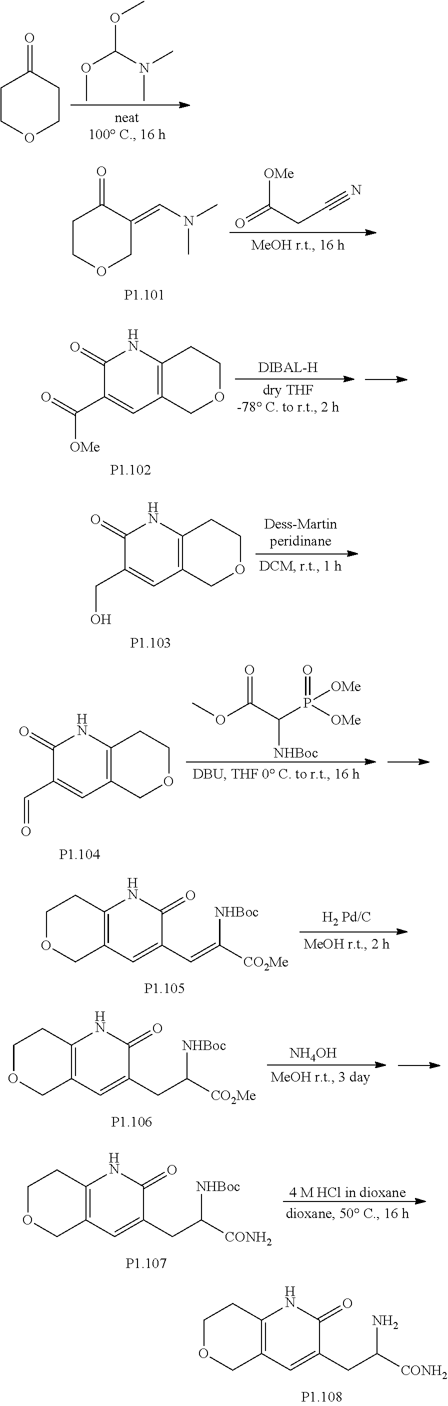

### Preparation 106. (E)-3-((dimethylamino)methylene)tetrahydro-4H-pyran-4-one (P1.101)

A solution of tetrahydro-4H-pyran-4-one (5.00 g, 49.9 mmol, 1.0 eq) in 1,1-dimethoxy-N,N-dimethylmethanamine (5.95 g, 49.9 mmol, 1.0 eq) was stirred at 100° C. for 16 h. The reaction solution was concentrated under reduced pressure to afford crude P1.101 as a brown-red oil. ¹H NMR (400 MHz, CDCl₃), δ: 7.47 (s, 1H), 4.76 (s, 2H), 3.90 (t, J=6.0 Hz, 2H), 3.02 (s, 6H), 2.43 (t, J=6.0 Hz, 2H).

### Preparation 107. Methyl 2-oxo-1,5,7,8-tetrahydro-2H-pyrano[4,3-b]pyridine-3-carboxylate (P1.102)

To a solution of P1.101 (crude, 49.9 mmol, 1.0 eq) in anh. methanol (70 mL) was added methyl 2-cyanoacetate (5.44 g, 54.9 mmol, 1.1 eq) at rt. After the reaction solution was stirred at rt for 16 h, and concentrated under reduced pressure, the residue was purified by silica gel chromatography (5% methanol in dichloromethane) to afford P1.102 (3.95 g, 38%) as a red solid. ¹H NMR (400 MHz, DMSO-d₆), δ: 12.03 (br. s, 1H), 7.82 (s, 1H), 4.42 (s, 2H), 3.81 (t, J=5.6 Hz, 2H), 3.70 (s, 3H), 2.59 (t, J=5.6 Hz, 2H).

### Preparation 108. 3-(Hydroxymethyl)-1,5,7,8-tetrahydro-2H-pyrano[4,3-b]pyridin-2-one (P1.103)

To a solution of P1.102 (3.42 g, 16.3 mmol, 1.0 eq) in anh. THF (81 mL) was added 1.2 M diisobutylaluminium hydride in toluene solution (61.3 mL, 73.5 mmol, 4.5 eq) at −78° C. dropwise. After the reaction solution was stirred at −78° C. for 10 min and at 0° C. for another 2 h, the reaction mixture was quenched with NaHCO_{3(aq)}, extracted with EtOAc, and washed with water. The water layer was concentrated under reduced pressure to afford P1.103 (2.15 g, 72%) as a yellow solid which was used in next step without purification. ¹H NMR (400 MHz, CD₃OD), δ: 7.33 (s, 1H), 4.53 (s, 2H), 4.47 (s, 2H), 3.94 (t, J=5.6 Hz, 2H), 2.67-2.65 (m, 2H).

### Preparation 109. 2-Oxo-1,5,7,8-tetrahydro-2H-pyrano[4,3-b]pyridine-3-carbaldehyde (P1.104)

To a solution of P1.103 (1.32 g, 7.28 mmol, 1.0 eq) in DCM (73 mL) was added Dess-Martin periodinane (3.24 g, 7.65 mmol, 1.05 eq) at rt. After the reaction mixture was stirred at rt for 1 h, the solution was quenched by Na₂S₂O_{3(aq)} (2.2 mL) and stirred at rt for 16 h. The mixture solution was concentrated under reduced pressure and purified by silica gel chromatography (6% methanol in DCM) to afford P1.104 (2.12 g, crude) as a yellow solid. ¹H NMR (400 MHz, DMSO-d₆), δ: 10.04 (s, 1H), 7.73 (s, 1H), 4.45 (s, 2H), 3.83 (t, J=5.6 Hz, 2H), 2.66-2.63 (m, 2H).

### Preparation 110. Methyl (Z)-2-((tert-butoxycarbonyl)amino)-3-(2-oxo-1,5,7,8-tetrahydro-2H-pyrano[4,3-b]pyridin-3-yl)acrylate (P1.105)

To a solution of methyl 2-((tert-butoxycarbonyl)amino)-2-(dimethoxyphosphoryl)acetate (2.16 g, 7.28 mmol, 1.0 eq) in anh. THF (73 mL) was added 1,8-diazabicyclo[5.4.0]undec-7-ene (1.41 mL, 9.43 mmol, 1.3 eq) at 0° C. After the reaction solution was stirred at 0° C. for 30 min, the solution of P1.104 (crude, 7.28 mmol, 1.0 eq) in anh. THF (10 mL) was added at 0° C. and stirred at rt for 16 h. The reaction mixture was concentrated under reduced pressure and purified by silica gel chromatography (10% methanol in DCM) to afford P1.105 (1.84 g, 72%) as a yellow solid. ¹H NMR (400 MHz, DMSO-d₆), δ: 12.18 (br. s, 1H), 9.19 (br. s, 1H), 7.54 (s, 1H), 6.81 (s, 1H), 4.40 (s, 2H), 3.83 (t, J=5.6 Hz, 2H), 3.70 (s, 3H), 2.62-2.59 (m, 2H), 1.35 (s, 9H).

### Preparation 111. Methyl 2-((tert-butoxycarbonyl)amino)-3-(2-oxo-1,5,7,8-tetrahydro-2H-pyrano[4,3-b]pyridin-3-yl)propanoate (P1.106)

To a solution of P1.105 (1.99 g, 5.68 mmol, 1.0 eq) in methanol (50 mL) was added Pd/C (50% wet with water, 1.90 g, 50% w/w) at rt and degassed with hydrogen. After the reaction mixture was stirred at rt for 2 h under hydrogen atmosphere, the solution was filtered with Celite and washed with methanol. The filtrate was concentrated under reduced pressure and purified by C18 flash column (0-100% methanol in H₂O) to afford P1.106 (585 mg, 29%) as a yellow solid. ¹H NMR (400 MHz, DMSO-d₆), δ: 11.62 (br. s, 1H), 7.28 (d, J=7.6 Hz, 1H), 6.99 (s, 1H), 4.38-4.29 (m, 2H), 4.27-4.21 (m, 1H), 3.79 (t, J=5.6 Hz, 2H), 3.58 (s, 3H), 2.85-2.81 (m, 1H), 2.57-2.51 (m, 3H), 1.32 (s, 9H).

### Preparation 112. tert-Butyl (1-amino-i-oxo-3-(2-oxo-1,5,7,8-tetrahydro-2H-pyrano[4,3-b]pyridin-3-yl)propan-2-yl)carbamate (P1.107)

To a solution of P1.106 (585 mg, 1.66 mmol, 1.0 eq) in methanol (16.6 mL) was added 30% ammonium hydroxide solution (8.3 mL) and stirred at rt for 3 days. The solution was concentrated under reduced pressure to afford P1.107 (454 mg, 72%) as a white solid. ¹H NMR (400 MHz, DMSO-d₆), δ: 7.17-6.92 (m, 3H), 4.38-4.30 (m, 2H), 4.04-4.00 (m, 1H), 3.80-3.78 (m, 2H), 2.76-2.51 (m, 4H), 1.31-1.22 (s, 9H).

### Preparation 113. 2-Amino-3-(2-oxo-1,5,7,8-tetrahydro-2H-pyrano[4,3-b]pyridin-3-yl)propanamide (P1.108)

To a solution of P1.107 (349 mg, 1.03 mmol, 1.0 eq) in 1,4-dioxane (8.0 mL) was added 4 M hydrogen chloride in dioxane (1.03 mL, 4.13 mmol, 4.0 eq) and stirred at 50° C. for 16 h.

The reaction solution was concentrated under reduced pressure to afford P1.108 (HCl salt, 349 mg, crude) as a yellow solid. ¹H NMR (400 MHz, DMSO-d₆), δ: 11.93 (br. s, 1H), 8.35-8.34 (br. m, 3H), 7.90 (br. s, 1H), 7.49 (br. s, 1H), 7.12 (s, 1H), 4.41-4.33 (m, 2H), 3.98-3.95 (m, 1H), 3.83-3.80 (m, 2H), 2.92-2.80 (m, 2H), 2.57-2.54 (m, 2H).

### Synthesis of (1R,2S,5S)—N-(1-amino-i-oxo-3-(2-oxo-1,5,7,8-tetrahydro-2H-pyrano[4,3-b]pyridin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.109)

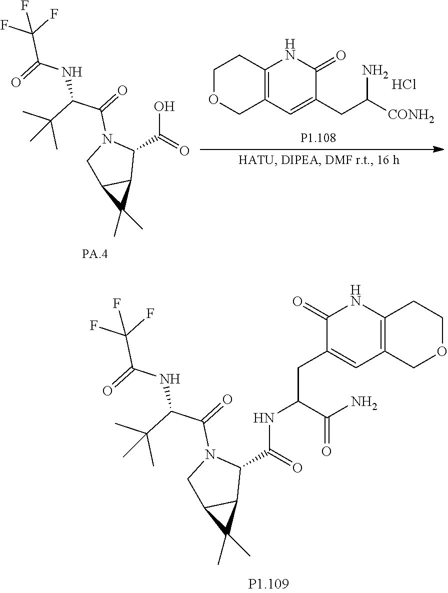 [SMILES: CC(C)(C)[C@H](NC(=O)C(F)(F)F)C(=O)N1C[C@H]2[C@@H]([C@H]1C(=O)O)C2(C)C ; NC(=O)C(N)Cc1cc2c([nH]c1=O)CCOC2 ; Cl ; CC(C)(C)[C@H](NC(=O)C(F)(F)F)C(=O)N1C[C@H]2[C@@H]([C@H]1C(=O)NC(Cc1cc3c([nH]c1=O)CCOC3)C(N)=O)C2(C)C]

### Preparation 114. (1R,2S,5S)—N-(1-amino-i-oxo-3-(2-oxo-1,5,7,8-tetrahydro-2H-pyrano[4,3-b]pyridin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.109)

To a solution of PA.4 (181 mg, 0.498 mmol, 1.0 eq) in N,N-dimethylformamide (3.3 ml) was added P1.108 (142 mg, 0.598 mol, 1.2 eq), 1-[bis(dimethylamino)methylene]-1H-1,2,3-triazolo[4,5-b]pyridinium 3-oxide hexafluorophosphate (208 mg, 0.548 mmol, 1.1 eq) and N,N-diisopropylethylamine (0.260 ml, 1.49 mmol, 3.0 eq) at rt. After the reaction mixture was stirred at rt for 16 h, the mixture was purified by C18 flash column (5-100% methanol in H₂O) to afford P1.109 (176 mg, 60%) as a white solid. ¹H NMR (400 MHz, DMSO-d₆), δ: 11.73-11.61 (m, 2H), 9.50-9.37 (m, 2H), 8.65-8.52 (m, 2H), 7.43-6.88 (m, 6H), 4.42-3.56 (m, 18H), 3.16-3.15 (m, 2H), 2.74-2.71 (m, 2H), 2.01-1.96 (m, 1H), 1.51-1.43 (m, 2H), 1.31-1.22 (m, 2H), 1.04-0.80 (m, 31H).

### Synthesis of (1R,2S,5S)—N-(1-amino-i-oxo-3-(2-oxo-1,5,7,8-tetrahydro-2H-pyrano[4,3-b]pyridin-3-yl)propan-2-yl)-3-((S)-3,3-dimethyl-2-(methylsulfonamido)butanoyl)-6,6-dimethyl-3-azabicyclo[3.1.0]hexane-2-carboxamide (P1.110)

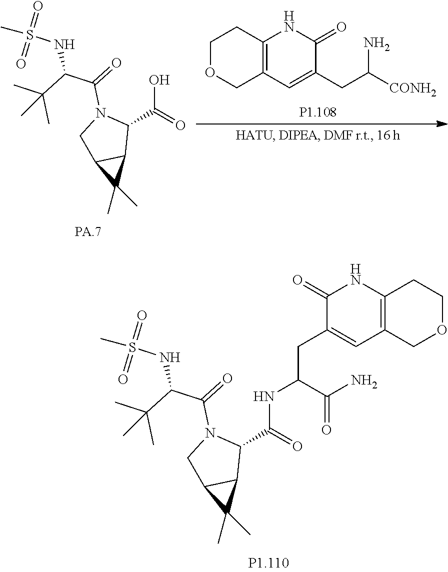 [SMILES: CC(C)(C)[C@H](NS(C)(=O)=O)C(=O)N1C[C@H]2[C@@H]([C@H]1C(=O)O)C2(C)C ; NC(=O)C(N)Cc1cc2c([nH]c1=O)CCOC2 ; CC(C)(C)[C@H](NS(C)(=O)=O)C(=O)N1C[C@H]2[C@@H]([C@H]1C(=O)NC(Cc1cc3c([nH]c1=O)CCOC3)C(N)=O)C2(C)C]

### Preparation 115. (1R,2S,5S)—N-(1-amino-i-oxo-3-(2-oxo-1,5,7,8-tetrahydro-2H-pyrano[4,3-b]pyridin-3-yl)propan-2-yl)-3-((S)-3,3-dimethyl-2-methylsulfonamido)-butanoyl)-6,6-dimethyl-3-azabicyclo[3.1.0]hexane-2-carboxamide (P1.110)

To a solution of PA.7 (368 mg, 1.06 mmol, 1.0 eq) in N,N-dimethylformamide (7 ml) was added P1.108 (349 mg, 1.27 mol, 1.2 eq), N,N-diisopropylethylamine (0.555 ml, 3.18 mmol, 3.0 eq) and 1-[bis(dimethylamino)methylene]-1H-1,2,3-triazolo[4,5-b]pyridinium 3-oxide hexafluorophosphate (444 mg, 1.16 mmol, 1.1 eq) at rt. After the reaction mixture was stirred at rt for 16 h, the mixture was purified by C18 flash column (5-100% methanol in H₂O) to afford P1.110 (306 mg, 53%) as an orange solid. ¹H NMR (400 MHz, CD₃OD), δ: 7.22-7.16 (m, 2H), 4.64-4.47 (m, 6H), 4.27-4.23 (m, 2H), 4.02-3.79 (m, 10H), 3.20-2.99 (m, 2H), 2.90-2.75 (m, 8H), 2.64-2.63 (m, 4H), 1.60-1.57 (m, 2H), 1.44-1.18 (m, 2H), 1.07-0.94 (m, 30H).

### Synthesis of (1R,2S,5S)—N-(1-amino-3-(6,6-difluoro-2-oxo-1,2,5,6,7,8-hexahydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.120)

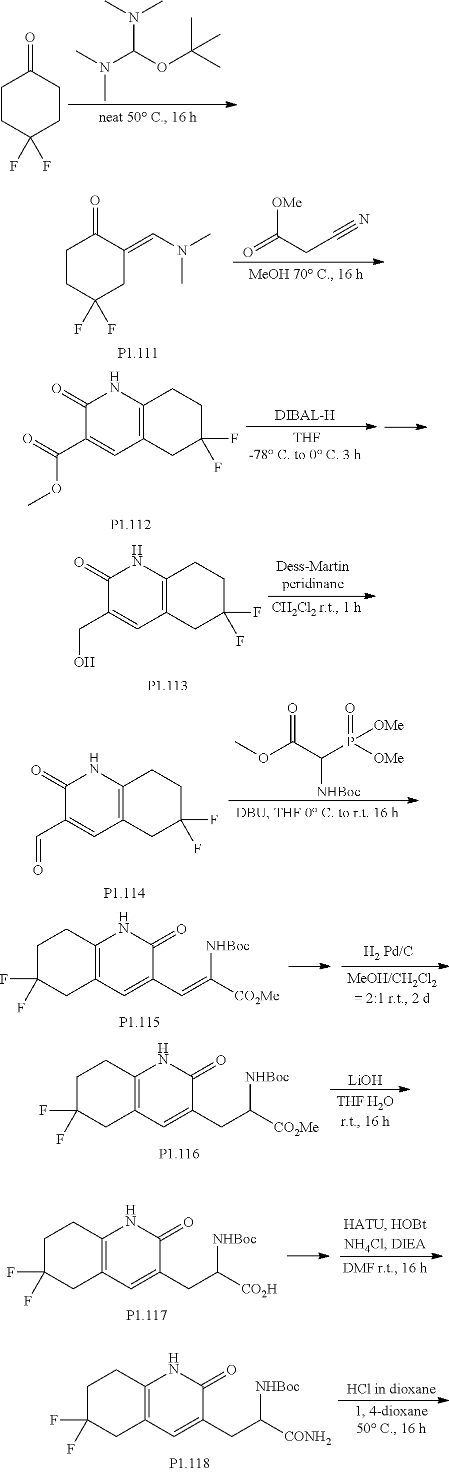

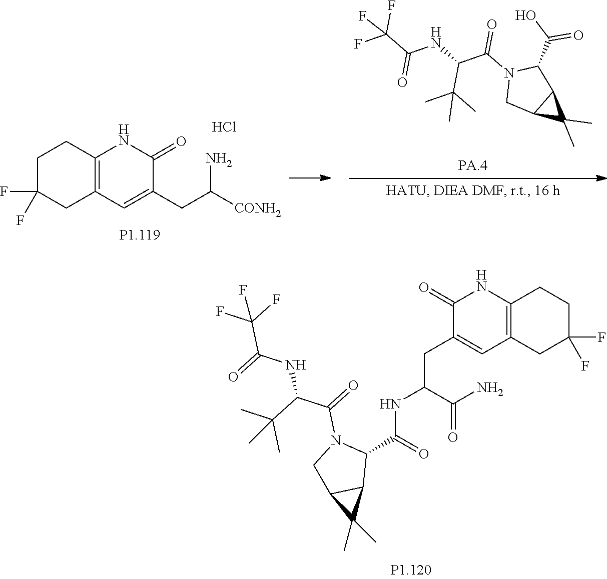 [SMILES: NC(=O)C(N)Cc1cc2c([nH]c1=O)CCC(F)(F)C2 ; Cl ; CC(C)(C)[C@H](NC(=O)C(F)(F)F)C(=O)N1C[C@H]2[C@@H]([C@H]1C(=O)O)C2(C)C ; CC(C)(C)[C@H](NC(=O)C(F)(F)F)C(=O)N1C[C@H]2[C@@H]([C@H]1C(=O)NC(Cc1cc3c([nH]c1=O)CCC(F)(F)C3)C(N)=O)C2(C)C]

### Preparation 116. (E)-2-((Dimethylamino)methylene)-4,4-difluorocyclohexan-1-one (P1.111)

A mixture of 4,4-difluorocyclohexanone (10.30 g, 76.79 mmol, 1.0 eq) and 1-tert-butoxy-N,N,N′,N′-tetramethylmethanediamine (19.0 mL, 92.15 mmol, 1.2 eq) was stirred at 50° C. for 16 h. The mixture was cooled to rt. The reaction mixture was concentrated to give P1.111 as a brown oil which was used in the next step without further purification. ¹H NMR (400 MHz, CDCl₃), δ: 7.54 (s, 1H), 3.10 (s, 6H), 2.53 (t, J=7.2 Hz, 2H), 2.30-2.15 (m, 4H).

### Preparation 117. Methyl 6,6-difluoro-2-oxo-1,2,5,6,7,8-hexahydroquinoline-3-carboxylate (P1.112)

To a solution of P1.111 (crude, 76.79 mmol, 1.0 eq) in methanol (110 mL) was added methyl 2-cyanoacetate (7.45 mL, 84.5 mmol, 1.1 eq). After the reaction was stirred at 70° C. for 16 h, the reaction mixture was concentrated to give a residue, which was purified by silica gel column chromatography (DCM:MeOH=50:1 to 30:1) to give P1.112 (5.2 g, 28% yield) as a red-brown solid. ¹H NMR (400 MHz, CDCl₃), δ: 7.99 (s, 1H), 3.92 (s, 3H), 3.15-3.03 (m, 4H), 2.34-2.22 (m, 2H).

### Preparation 118. 6,6-Difluoro-3-(hydroxymethyl)-5,6,7,8-tetrahydroquinolin-2(1H)-one (P1.113)

To a solution of P1.112 (4.30 g, 17.68 mmol, 1.0 eq) in THF (110 mL) was added 1.2 M diisobutylaluminium hydride in toluene (DIBAL-H, 66.3 mL, 79.56 mmol, 4.5 eq) dropwise at −78° C. The reaction was stirred at −78° C. for 15 min and then stirred at 0° C. for 3 h. The mixture was treated with sat. NaHCO_{3(aq)} and extracted with ethyl acetate three times. The combined organic layers were dried over Na₂SO₄₍ₛ₎, filtered, and concentrated in vacuo to give the residue, which was purified by silica gel column chromatography (DCM:MeOH=20:1) to give P1.113 (2.19 g, 58% yield) as a light-yellow solid. ¹H NMR (400 MHz, CD₃OD), δ: 7.39 (s, 1H), 4.47 (s, 2H), 3.04 (t, J=14.4 Hz, 2H), 2.84 (t, J=7.2 Hz, 2H), 2.31-2.18 (m, 2H).

### Preparation 119. 6,6-Difluoro-2-oxo-1,2,5,6,7,8-hexahydroquinoline-3-carbaldehyde (P1.114)

To a solution of P1.113 (2.19 g, 10.2 mmol, 1.0 eq) in DCM (100 mL) was added Dess-Martin periodinane (4.53 g, 10.7 mmol, 1.05 eq), and stirred at rt for 1 h. To the reaction mixture was added 1 M Na₂S₂O_{3(aq)} (15.3 mL, 15.27 mmol, 1.5 eq), and then stirred at rt for another 1 h. The mixture was filtered and the filtrate was concentrated. The residue was purified by silica gel column chromatography (DCM:MeOH=40:1) and the product was washed with diethyl ether to give P1.114 (2.57 g) as a light-yellow solid. ¹H NMR (400 MHz, CDCl₃), δ: 13.15 (br. s, 1H), 10.27 (s, 1H), 7.90 (s, 1H), 3.15-3.01 (m, 4H), 2.36-2.23 (m, 2H).

### Preparation 120. Methyl (Z)-2-((tert-butoxycarbonyl)amino)-3-(6,6-difluoro-2-oxo-1,2,5,6,7,8-hexahydroquinolin-3-yl)acrylate (P1.115)

To a solution of (±)-Boc-α-phosphonoglycine trimethyl ester (4.73 g, 15.9 mmol, 1.3 eq) in anh. THF (32 mL) was added DBU (2.40 mL, 15.9 mmol, 1.3 eq) slowly at 0° C. After the reaction was stirred at 0° C. for 30 min, the ylide solution was added slowly into a solution of P1.115 (2.57 g, 12.06 mmol, 1.0 eq) in anh. THF (50 mL) at 0° C. The mixture was stirred at rt for 16 h. The solvent was removed under reduced pressure. The residue was purified by silica gel column chromatography (DCM:MeOH=50:1 to 40:1) to give P1.115 (1.2 g, 26% yield) as a light-yellow solid. ¹H NMR (400 MHz, CDCl₃), δ: 13.30 (br. s, 1H), 8.93 (s, 1H), 7.34 (s, 1H), 6.58 (s, 1H), 3.86 (s, 3H), 3.14-2.98 (m, 4H), 2.36-2.22 (m, 2H), 1.43 (s, 9H).

### Preparation 121. Methyl 2-((tert-butoxycarbonyl)amino)-3-(6,6-difluoro-2-oxo-1,2,5,6,7,8-hexahydroquinolin-3-yl)propanoate (P1.116)

To a solution of P1.115 (1.20 g, 3.12 mmol, 1.0 eq) in methanol (20 mL) and DCM (10 mL) was added Pd/C powder (500 mg, 10% wt). The reaction was stirred at rt for 2 days under hydrogen balloon (1 atm). The solution was filtered through a pad of celite. The filtrate was concentrated under reduced pressure to give P1.116 (1.18 g, 98% yield) as a white solid. ¹H NMR (400 MHz, CDCl₃), δ: 12.91 (br. s, 1H), 7.07 (s, 1H), 6.53-6.35 (m, 1H), 4.50-4.25 (m, 1H), 3.71 (s, 3H), 3.04-2.86 (m, 6H), 2.30-2.16 (m, 2H), 1.40 (s, 9H).

### Preparation 122. 2-((tert-Butoxycarbonyl)amino)-3-(6,6-difluoro-2-oxo-1,2,5,6,7,8-hexahydroquinolin-3-yl)propanoic acid (P1.117)

To a solution of P1.116 (1.18 g, 3.05 mmol, 1.0 eq) in tetrahydrofuran (14 mL) and water (14 mL) was added lithium hydroxide (219.4 mg, 9.16 mmol, 3.0 eq). The reaction was stirred at room temperature for 16 h. The mixture was acidified by slow addition of 1 N HCl_{(aq)}, adjusted pH to 6, extracted with dichloromethane three times. The combined organic layers were dried over Na₂SO₄₍ₛ₎, filtered, and concentrated in vacuo to give crude P1.117 (924 mg, 81% yield) as a pink solid. ¹H NMR (400 MHz, DMSO-d₆), δ: 11.64 (br. s, 1H), 7.14 (d, J=8.0 Hz, 1H), 7.07 (s, 1H), 4.20-4.12 (m, 1H), 3.01-2.80 (m, 3H), 2.73-2.65 (m, 1H), 2.58-2.52 (m, 1H), 2.25-2.12 (m, 2H), 1.38-1.23 (m, 9H).

### Preparation 123. tert-Butyl (1-amino-3-(6,6-difluoro-2-oxo-1,2,5,6,7,8-hexahydroquinolin-3-yl)-1-oxopropan-2-yl)carbamate (P1.118)

To a solution of P1.117 (924.0 mg, 2.48 mmol, 1.0 eq) in dimethylformamide (9.2 mL) was added HATU (1.13 g, 2.98 mmol, 1.2 eq), HOBt (190.0 mg, 1.24 mmol, 0.5 eq), ammonium chloride (199 mg, 3.72 mmol, 1.5 eq) and N,N-diisopropylethylamine (DIPEA, 2.2 mL, 12.41 mmol, 5.0 eq). The reaction was stirred at rt for 16 h. The mixture was poured into water and the precipitate was collected through filtration, washed with water, and dried to give P1.118 (664 mg, 72% yield) as a white solid. ¹H NMR (400 MHz, DMSO-d₆), δ: 11.64 (s, 1H), 7.21 (s, 1H), 7.05 (s, 1H), 6.95 (d, J=7.2 Hz, 2H), 4.09-3.98 (m, 1H), 3.03-2.86 (m, 2H), 2.79-2.65 (m, 3H), 2.57-2.51 (m, 1H), 2.26-2.12 (m, 2H), 1.31 (s, 9H).

### Preparation 124. 2-Amino-3-(6,6-difluoro-2-oxo-1,2,5,6,7,8-hexahydroquinolin-3-yl)propanamide hydrochloride (P1.119)

To a mixture of P1.118 (664.0 mg, 1.79 mmol, 1.0 eq) in 1,4-dioxane (16 mL) was added 4 M HCl in 1,4-dioxane (2.2 mL, 8.94 mmol, 5.0 eq). The reaction was stirred at 50° C. for 16 h. The solvent was removed under reduced pressure to give P1.119 (732 mg, crude) as a light yellow solid which was used in the next step without further purification. ¹H NMR (400 MHz, DMSO-d₆): δ 11.96 (br. s, 1H), 8.33 (s, 3H), 7.92 (s, 1H), 7.51 (s, 1H), 7.19 (s, 1H), 4.05-3.95 (m, 1H), 3.03-2.79 (m, 4H), 2.73 (t, J=6.8 Hz, 2H), 2.29-2.14 (m, 2H).

### Preparation 125. (1R,2S,5S)—N-(1-Amino-3-(6,6-difluoro-2-oxo-1,2,5,6,7,8-hexahydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.120)

To a solution of PA.4 (0.10 g, 0.27 mmol), P1.119 (0.10 g, 0.33 mmol), and DIPEA (0.15 mL, 0.80 mmol) in DMF (3.0 mL) was added HATU (115 mg, 0.302 mmol) at 0° C. The reaction mixture was stirred at room temperature for 16 h. The solution was purified by C18 column (5-100% MeOH in H₂O) to give P1.120 (0.10 g, 60%) as a white solid. ¹H NMR (400 MHz, DMSO-d₆), δ: 11.83-11.53 (m, 2H), 9.53-9.29 (m, 2H), 8.71-8.42 (m, 2H), 7.26-6.76 (m, 6H), 4.47-4.17 (m, 4H), 4.14-4.06 (m, 2H), 4.04-3.83 (m, 2H), 3.71-3.52 (m, 2H), 3.07-2.61 (m, 12H), 2.28-2.08 (m, 4H), 1.54-1.40 (m, 2H), 1.28 (d, J=7.8 Hz, 1H), 1.08-1.02 (m, 1H), 1.00-0.93 (m, 24H), 0.86-0.78 (m, 6H).

### Synthesis of (1R,2S,5S)—N-(1-amino-3-(6,6-difluoro-2-oxo-1,2,5,6,7,8-hexahydroquinolin-3-yl)-1-oxopropan-2-yl)-3-((S)-3,3-dimethyl-2-(methylsulfonamido)butanoyl)-6,6-dimethyl-3-azabicyclo[3.1.0]hexane-2-carboxamide (P1.121)

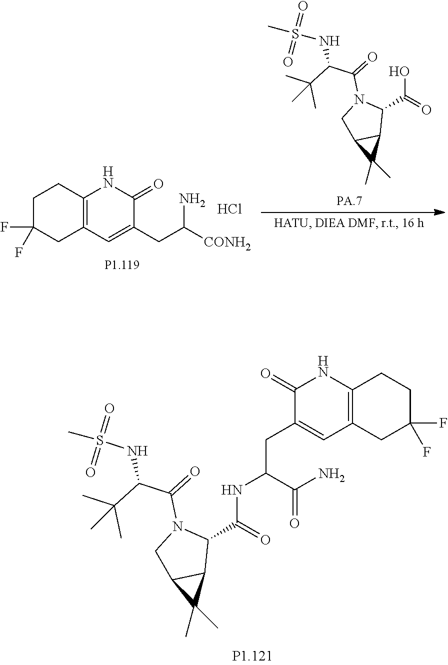 [SMILES: NC(=O)C(N)Cc1cc2c([nH]c1=O)CCC(F)(F)C2 ; Cl ; CC(C)(C)[C@H](NS(C)(=O)=O)C(=O)N1C[C@H]2[C@@H]([C@H]1C(=O)O)C2(C)C ; CC(C)(C)[C@H](NS(C)(=O)=O)C(=O)N1C[C@H]2[C@@H]([C@H]1C(=O)NC(Cc1cc3c([nH]c1=O)CCC(F)(F)C3)C(N)=O)C2(C)C]

### Preparation 126. (1R,2S,5S)—N-(1-amino-3-(6,6-difluoro-2-oxo-1,2,5,6,7,8-hexahydroquinolin-3-yl)-1-oxopropan-2-yl)-3-((S)-3,3-dimethyl-2-(methylsulfonamido)butanoyl)-6,6-dimethyl-3-azabicyclo[3.1.0]hexane-2-carboxamide (P1.121)

To a solution of PA.7 (150.0 mg, 0.260 mmol, 1.0 eq) in DMF (2 mL) was added P1.119 (95.9 mg, 0.312 mmol, 1.2 eq), HATU (108.7 mg, 0.286 mmol, 1.1 eq) and DIPEA (0.14 mL, 0.779 mmol, 3.0 eq). The reaction mixture was stirred at rt for 16 h. The mixture was concentrated and purified by C-18 reversed-phase column chromatography (mobile phase A: water (with 0.1% NH₄HCO₃), mobile phase B: methanol, UV: 214 and 254 nm, Flow rate: 35 mL/min, Gradient: 3-90% (% B)) to give P1.121 (115 mg, 74% yield) as a white solid. ¹H NMR (400 MHz, DMSO-d₆), δ: 11.72-11.57 (m, 1H), 8.71 (d, J=8.4 Hz, 0.5H), 8.40 (d, J=6.8 Hz, 0.5H), 7.25-6.89 (m, 4H), 4.37-4.29 (m, 0.5H), 4.26-4.19 (m, 0.5H), 4.14-4.11 (m, 1H), 3.88-3.76 (m, 2H), 3.70-3.62 (m, 1H), 3.17 (s, 0.5H), 3.16 (s, 0.5H), 3.09-2.39 (m, 9H), 2.26-2.12 (m, 2H), 1.53-1.51 (m, 1H), 1.26 (d, J=7.6 Hz, 0.5H), 1.01-0.88 (m, 15.5H).

### Synthesis of (1R,2S,5S)—N-(1-Amino-3-(6,6-dimethyl-2-oxo-1,2,5,6,7,8-hexahydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.131)

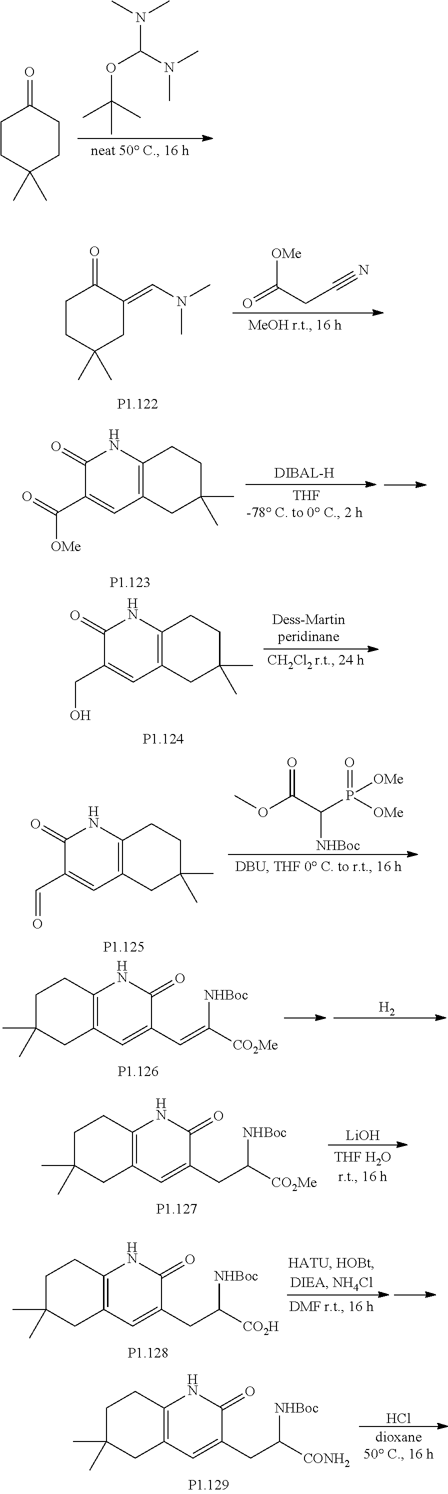

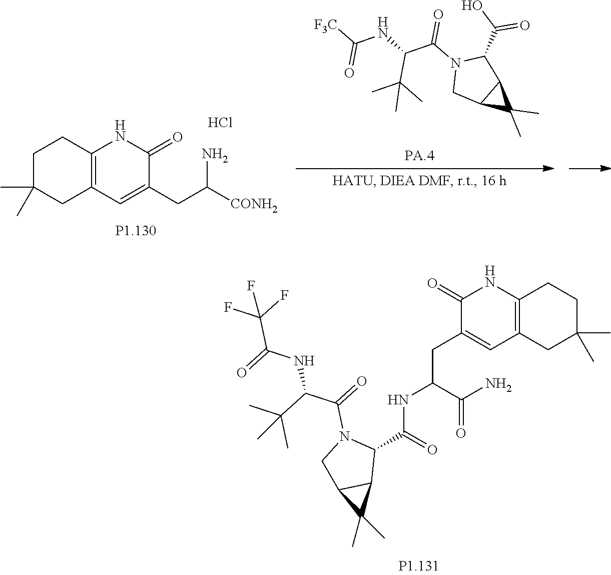 [SMILES: Cl ; CC(C)(C)[C@H](NC(=O)C(F)(F)F)C(=O)N1C[C@H]2[C@@H]([C@H]1C(=O)O)C2(C)C ; CC1(C)CCc2[nH]c(=O)c(CC(N)C(N)=O)cc2C1 ; CC1(C)CCc2[nH]c(=O)c(CC(NC(=O)[C@@H]3[C@@H]4[C@H](CN3C(=O)[C@@H](NC(=O)C(F)(F)F)C(C)(C)C)C4(C)C)C(N)=O)cc2C1]

### Preparation 127. (E)-2-((dimethylamino)methylene)-4,4-dimethylcyclohexan-1-one (P1.122)

A solution of 4,4-dimethylcyclohexanone (2.00 g, 15.9 mmol, 1.0 eq) and tert-butoxy bis(dimethylamino)methane (4.3 mL, 19 mmol, 1.2 eq) was stirred for 16 h at 50° C. The mixture was concentrated to give P1.122 as a crude product which was used in the next step without further purification.

### Preparation 128. Methyl 6,6-dimethyl-2-oxo-1,2,5,6,7,8-hexahydroquinoline-3-carboxylate (P1.123)

A solution of crude P1.122 (crude 15.9 mmol, 1.0 eq) and methyl cyanoacetate (1.53 mL, 17.4 mmol, 1.1 eq) was stirred for 16 h at rt and then concentrated. The residue was purified by silica gel chromatography (0-10% MeOH in DCM) to give P1.123 (1 g, 27% yield with 2 steps) as a purple solid. ¹H NMR (400 MHz, CDCl₃), δ: 7.98 (s, 1H), 3.89 (s, 3H), 2.80 (t, J=6.4 Hz, 2H), 2.33 (s, 2H), 1.59 (t, J=6.4 Hz, 2H), 0.99 (s, 6H).

### Preparation 129. 3-(Hydroxymethyl)-6,6-dimethyl-5,6,7,8-tetrahydroquinolin-2(1H)-one (P1.124)

To a solution of P1.123 (1.02 g, 10.1 mmol, 1.5 eq) in THF (10 mL) was added DIBAL-H (1.00 g, 6.71 mmol, 1.0 eq) at −78° C. After stirring at 0° C. for 2 h, the reaction mixture was quenched with NaHCO_{3(aq)} and extracted with EtOAc. The combined organic layers were dried over Na₂SO₄₍ₛ₎, filtered, and concentrated under reduced pressure to give P1.124 (2.61 g, crude) as a light yellow solid which was used in the next step without purification. ¹H NMR (400 MHz, CDCl₃), δ: 7.19 (s, 1H), 4.58 (s, 2H), 2.68 (t, J=5.6 Hz, 2H), 2.23 (s, 2H), 1.57 (t, J=5.6 Hz, 2H), 0.99 (s, 6H).

### Preparation 130. 6,6-Dimethyl-2-oxo-1,2,5,6,7,8-hexahydroquinoline-3-carbaldehyde (P1.125)

To a solution of P1.124 (2.11 g, 10.2 mmol) in DCM (35 mL) was added Dess-Martin periodinane (4.53 g, 10.7 mmol) at rt. After the reaction mixture was stirred at rt for 24 h, the reaction mixture was quenched with Na₂S₂O₃ and concentrated. The residue was washed with DCM and filtered. The filtrate was concentrated under reduced pressure. The crude product was washed Et₂O to give P1.125 (2 g, 100%). ¹H NMR (400 MHz, CDCl₃), δ: 10.29 (s, 1H), 7.88 (s, 1H), 2.80 (t, J=6.8 Hz, 2H), 2.35 (s, 2H), 1.61 (t, J=6.8 Hz, 2H), 1.01 (s, 6H).

### Preparation 131. Methyl (Z)-2-((tert-butoxycarbonyl)amino)-3-(6,6-dimethyl-2-oxo-1,2,5,6,7,8-hexahydroquinolin-3-yl)acrylate (P1.126)

To a solution of (±)-Boc-α-phosphonoglycine trimethyl ester (3.35 g, 11.3 mmol, 1.1 eq) in anh. THF (52 mL) was added DBU (1.90 mL, 13.3 mmol, 1.3 eq) slowly at 0° C. After the reaction was stirred at 0° C. for 30 min, the ylide solution was added slowly into a solution of P1.125 (2.10 g, 10.23 mmol, 1.0 eq) in anh. THF (50 mL) at 0° C. The mixture was warmed to rt and stirred for 16 h. The solvent was removed under reduced pressure. The residue was purified by silica gel column chromatography (n-hexane:EtOAc=3:1 to 1:19) to give P1.126 (1.9 g, 50% yield) as a light-yellow solid. ¹H NMR (400 MHz, CDCl₃), δ: 12.56 (br. s, 1H), 9.09 (br. s, 1H), 7.33 (s, 1H), 6.60 (s, 1H), 3.85 (s, 3H), 2.81 (t, J=6.4 Hz, 2H), 2.31 (s, 2H), 1.61 (t, J=6.4 Hz, 2H), 1.43 (s, 9H), 1.00 (s, 6H).

### Preparation 132. Methyl 2-((tert-butoxycarbonyl)amino)-3-(6,6-dimethyl-2-oxo-1,2,5,6,7,8-hexahydroquinolin-3-yl)propanoate (P1.127)

To a solution of P1.126 (1.90 g, 5.05 mmol, 1.0 eq) in methanol (50 mL) was added Pd/C powder (190 mg, 10% wt). The reaction was stirred at rt for 16 h under hydrogen balloon (1 atm). The solution was filtered through a pad of celite. The filtrate was removed under reduced pressure to give P1.127 (1.89 g, 99% yield) as a white solid. ¹H NMR (400 MHz, CDCl₃), δ: 11.78 (br. s, 1H), 7.02 (s, 1H), 6.78 (d, J=7.2 Hz, 1H), 4.40-4.38 (m, 1H), 3.69 (s, 3H), 2.94-2.92 (m, 2H), 2.67 (t, J=6.8 Hz, 2H), 2.23 (s, 2H), 1.55 (t, J=6.8 Hz, 2H), 1.41 (s, 9H), 0.97 (s, 6H).

### Preparation 133. 2-((tert-Butoxycarbonyl)amino)-3-(6,6-dimethyl-2-oxo-1,2,5,6,7,8-hexahydroquinolin-3-yl)propanoic acid (P1.128)

To a solution of P1.127 (1.51 g, 3.99 mmol, 1.0 eq) in THF (18 mL) and water (18 mL) was added lithium hydroxide (286.6 mg, 11.97 mmol, 3.0 eq). The reaction was stirred at rt for 16 h. The mixture was acidified by slow addition of 1 N HCl_{(aq)}, adjusted pH to 6, and extracted with DCM three times. The combined organic layers were dried over Na₂SO₄₍ₛ₎, filtered, and concentrated in vacuo to give crude P1.128 (1.50 g, crude) as a white foam. ¹H NMR (400 MHz, DMSO-d₆), δ: 11.46 (br. s, 1H), 7.20 (d, J=7.2 Hz, 1H), 7.03 (s, 1H), 4.16-4.06 (m, 1H), 2.83-2.81 (m, 1H), 2.58-2.52 (m, 1H), 2.44 (t, J=5.6 Hz, 2H), 2.14 (s, 2H), 1.43 (t, J=6.8 Hz, 2H), 1.35 (s, 9H), 0.91 (s, 6H).

### Preparation 134. tert-Butyl (1-amino-3-(6,6-dimethyl-2-oxo-1,2,5,6,7,8-hexahydroquinolin-3-yl)-1-oxopropan-2-yl)carbamate (P1.129)

To a solution of P1.128 (1.45 g, 3.98 mmol, 1.0 eq) in DMF (15 mL) was added HATU (1.82 g, 4.77 mmol, 1.2 eq), HOBt (304.7 mg, 1.99 mmol, 0.5 eq), ammonium chloride (319.2 mg, 5.97 mmol, 1.5 eq) and DIPEA (3.5 mL, 19.89 mmol, 5.0 eq). The reaction was stirred at rt for 16 h. The mixture was concentrated in vacuo to get a residue, which was purified by C-18 reversed-phase column chromatography (mobile phase A: water (with 0.1% NH₄HCO₃), mobile phase B: methanol, UV: 214 and 254 nm, Flow rate: 40 mL/min, Gradient: 3-90% (% B)) to give P1.129 (1.21 g, 83% yield) as a white solid. ¹H NMR (400 MHz, DMSO-d₆), δ: 11.46 (s, 1H), 7.20 (s, 1H), 7.04 (d, J=7.6 Hz, 1H), 6.99 (s, 1H), 6.93 (s, 1H), 4.05-3.96 (m, 1H), 2.72-2.70 (m, 1H), 2.58-2.52 (m, 1H), 2.44 (t, J=6.4 Hz, 2H), 2.15 (s, 2H), 1.43 (t, J=6.4 Hz, 2H), 1.32 (s, 9H), 0.91 (s, 6H).

### Preparation 135. 2-Amino-3-(6,6-dimethyl-2-oxo-1,2,5,6,7,8-hexahydroquinolin-3-yl)propanamide hydrochloride (P1.130)

To a mixture of P1.129 (1.21 g, 3.33 mmol, 1.0 eq) in 1,4-dioxane (24 mL) was added 4 M HCl in 1,4-dioxane (4.2 mL, 16.7 mmol, 5.0 eq). The reaction was stirred at 50° C. for 16 h. The solvent was removed under reduced pressure to give P1.130 (1.28 g, crude) as a light yellow solid which was used in next step without further purification. ¹H NMR (400 MHz, DMSO-d₆), δ: 11.84 (br. s, 1H), 8.51 (br. s, 3H), 7.93 (s, 1H), 7.52 (s, 1H), 7.14 (s, 1H), 4.03-3.92 (m, 1H), 2.94-2.78 (m, 2H), 2.49-2.45 (m, 2H), 2.18 (s, 2H), 1.46 (t, J=6.4 Hz, 2H), 0.93 (s, 3H), 0.91 (s, 3H).

### Preparation 136. (1R,2S,5S)—N-(1-amino-3-(6,6-dimethyl-2-oxo-1,2,5,6,7,8-hexahydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.131)

To a solution of PA.4 (100.0 mg, 0.274 mmol, 1.0 eq) in DMF (2 mL) was added P1.130 (98.7 mg, 0.329 mmol, 1.2 eq), HATU (114.8 mg, 0.302 mmol, 1.1 eq) and DIPEA (0.14 mL, 0.823 mmol, 3.0 eq). The reaction mixture was stirred at rt for 16 h. The mixture was concentrated and purified by C-18 reversed-phase column chromatography (mobile phase A: water (with 0.1% NH₄HCO₃), mobile phase B: methanol, UV: 214 and 254 nm, Flow rate: 35 mL/min, Gradient: 3-90% (% B)) to give P1.131 (118 mg, 71% yield) as a white solid. ¹H NMR (400 MHz, DMSO-d₆), δ: 11.62 (br. s, 0.5H), 11.44 (br. s, 0.5H), 9.53 (d, J=8.8 Hz, 0.5H), 9.40 (d, J=8.4 Hz, 0.5H), 8.74 (d, J=8.0 Hz, 0.5H), 8.66 (d, J=6.4 Hz, 0.5H), 7.19 (s, 0.5H), 7.06 (s, 0.5H), 7.02 (s, 0.5H), 6.95 (s, 0.5H), 6.91 (s, 0.5H), 6.88 (s, 0.5H), 4.44 (d, J=8.4 Hz, 0.5H), 4.36-3.86 (m, 4H), 3.65-3.57 (m, 1H), 3.03-2.96 (m, 0.5H), 2.76-2.71 (m, 0.5H), 2.48-2.41 (m, 2H), 2.23-2.07 (m, 2H), 1.55-1.39 (m, 3H), 1.28 (d, J=8.0 Hz, 0.5H), 1.05-0.83 (m, 21.5H).

### Synthesis of (1R,2S,5S)—N-(1-amino-3-(6,6-dimethyl-2-oxo-1,2,5,6,7,8-hexahydroquinolin-3-yl)-1-oxopropan-2-yl)-3-((S)-3,3-dimethyl-2-(methylsulfonamido)butanoyl)-6,6-dimethyl-3-azabicyclo[3.1.0]hexane-2-carboxamide (P1.132)

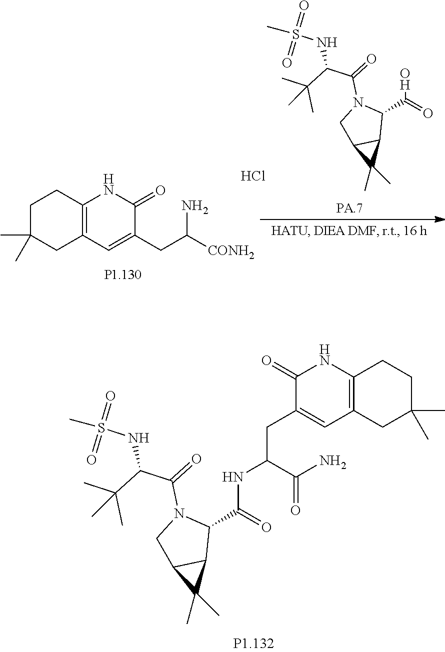

### Preparation 137. (1R,2S,5S)—N-(1-amino-3-(6,6-dimethyl-2-oxo-1,2,5,6,7,8-hexahydroquinolin-3-yl)-1-oxopropan-2-yl)-3-((S)-3,3-dimethyl-2-(methylsulfonamido)butanoyl)-6,6-dimethyl-3-azabicyclo[3.1.0]hexane-2-carboxamide (P1.131)

To a solution of PA.7 (150.0 mg, 0.260 mmol, 1.0 eq, 60%) in DMF (2 mL) was added P1.130 (93.5 mg, 0.312 mmol, 1.2 eq), HATU (108.7 mg, 0.286 mmol, 1.1 eq) and DIPEA (0.14 mL, 0.78 mmol, 3.0 eq). The reaction mixture was stirred at rt for 16 h. The mixture was concentrated and purified by C-18 reversed-phase column chromatography (mobile phase A: water (with 0.1% NH₄HCO₃), mobile phase B: methanol, UV: 214 and 254 nm, Flow rate: 40 mL/min, Gradient: 3-90% (% B)) to give P1.132 (109 mg, 71% yield) as a white solid. ¹H NMR (400 MHz, DMSO-d₆), δ: 11.51 (br. s, 0.5H), 11.43 (br. s, 0.5H), 8.80 (d, J=8.8 Hz, 0.5H), 8.49 (d, J=6.8 Hz, 0.5H), 7.25-6.85 (m, 4H), 4.37-4.28 (m, 0.5H), 4.24-4.16 (m, 0.5H), 4.12-4.09 (m, 1H), 3.92-3.74 (m, 2H), 3.71-3.62 (m, 1H), 3.10-3.02 (m, 0.5H), 2.85 (s, 1.5H), 2.84 (s, 1.5H), 2.77-2.71 (m, 0.5H), 2.47-2.40 (m, 2H), 2.24-2.09 (m, 2H), 1.56-1.49 (m, 1H), 1.47-1.39 (m, 2H), 1.26 (d, J=8.0 Hz, 0.5H), 1.01-0.86 (m, 21.5H).

### Synthesis of (1R,2S,5S)—N-(1-amino-3-(6-methyl-2-oxo-1,2-dihydroquinolin-3-yl)-1-oxopropan-2-yl)-3-((S)-3,3-dimethyl-2-((4-methylthiazol-2-yl)amino)butanoyl)-6,6-dimethyl-3-azabicyclo[3.1.0]hexane-2-carboxamide (P1.136)

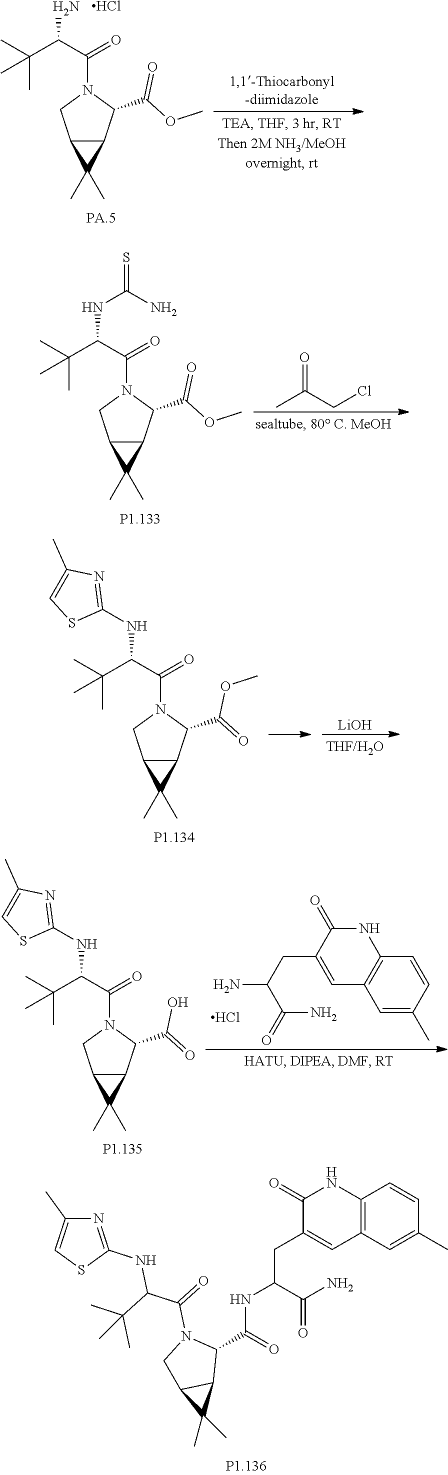

### Preparation 138. Methyl (1R,2S,5S)-3-((S)-3,3-dimethyl-2-thioureidobutanoyl)-6,6-dimethyl-3-azabicyclo[3.1.0]hexane-2-carboxylate (P1.133)

To a solution of PA.5 (1.0 g, 3.14 mmol, 1.0 eq) in dry THF (15 mL) was added triethylamine (1.09 mL, 7.85 mmol, 2.5 eq). After stirring for 15 min at rt, 1,1′-thiocarbonyldiimidazole (840 mg, 4.71 mmol, 1.5 eq) was added to reaction mixture. The resulting suspension was stirred for 3 h. Then 2M ammonia in methanol (1.57 ml, 3.14 mmol, 1.0 eq.) was added to the reaction mixture at 0° C. and resulting mixture was stirred at rt for overnight. The solvent was evaporated and residue was dissolved in ethyl acetate and washed with water. The combined organic layers were dried over magnesium sulfate, filtered, and concentrated. The crude was purified by flash silica gel column chromatography (mobile phase A: DCM, mobile phase B: methanol, UV: 214 and 254 nm, Flow rate: 50 mL/min, Gradient: 3-5% (% B) to give P1.133 (0.67 g, 62% yield) as a yellow solid. ¹H NMR (400 MHz, CDCl₃), δ: 7.54 (d, J=9.2 Hz, 1H), 6.52 (br. s, 2H), 4.98 (d, J=9.2 Hz, 1H), 4.24 (s, 1H), 4.22 (br. s, 1H), 3.84 (q, J=5.6 Hz, 1H), 3.82 (s, 3H), 1.43-1.32 (m, 2H), 0.95 (s, 9H), 0.94 (s, 3H), 0.85 (s, 3H).

### Preparation 139. Methyl (1R,2S,5S)-3-((S)-3,3-dimethyl-2-((4-methylthiazol-2-yl)amino)butanoyl)-6,6-dimethyl-3-azabicyclo[3.1.0]hexane-2-carboxylate (P1.134)

To a solution of P1.133 (600 mg, 1.76 mmol, 1.0 eq) in methanol (70 mL) was added 1-chloropropan-2-one (353 μL, 4.4 mmol, 2.5 eq) slowly at rt. The reaction mixture was heated at 80° C. for 3 days. The reaction mixture was concentrated to get crude product. The residue was purified by silica gel flash column chromatography (mobile phase A: DCM, mobile phase B: methanol, UV: 214 and 254 nm, Flow rate: 50 mL/min, Gradient: 2-3% (% B)) to give P1.134 (500 mg, 74% yield) as a white solid. ¹H NMR (400 MHz, MeOD-d₄): 6.13 (s, 1H), 4.49 (s, 1H), 4.33 (s, 1H), 4.16 (d, J=10.4 Hz, 1H), 4.00-3.97 (m, 1H), 3.74 (s, 3H), 2.16 (s, 3H), 1.60-1.58 (m, 1H), 1.46 (d, J=7.6 Hz, 1H), 1.10 (s, 9H), 1.05 (s, 3H), 0.85 (s, 3H).

### Preparation 140. (1R,2S,5S)-3-((S)-3,3-dimethyl-2-((4-methylthiazol-2-yl)amino)butanoyl)-6,6-dimethyl-3-azabicyclo[3.1.0]hexane-2-carboxylic acid (P1.135)

To a solution of P1.134 (100 mg, 0.263 mmol, 1.0 eq) in THF (0.526 mL) was added a solution of LiOH (18.93 mg, 0.79 mmol, 3.0 eq) in water (0.53 mL). The resulting mixture was stirred at rt for overnight. The mixture was neutralized with 1N HCl and extracted with ethyl acetate. The combined organic layers were dried over Na₂SO₄₍ₛ₎, filtered and concentrated in vacuo to give P1.135 (70 mg, 72% yield) as a white solid. ¹H NMR (400 MHz, MeOD-d₄), δ: 6.16 (s, 1H), 4.47 (s, 1H), 4.32 (s, 1H), 4.13 (d, J=10.8 Hz, 1H), 4.00-3.96 (m, 1H), 2.18 (s, 3H), 1.59 (dd, J=7.6, 5.2 Hz, 1H), 1.50 (d, J=7.6 Hz, 1H), 1.10 (s, 9H), 1.06 (s, 3H), 0.86 (s, 3H).

### Preparation 141. (1R,2S,5S)—N-(1-amino-3-(6-methyl-2-oxo-1,2-dihydroquinolin-3-yl)-1-oxopropan-2-yl)-3-((S)-3,3-dimethyl-2-((4-methylthiazol-2-yl)amino)butanoyl)-6,6-dimethyl-3-azabicyclo[3.1.0]hexane-2-carboxamide (P1.136)

To a solution of P1.135 (70 mg, 0.1915 mmol, 1.0 eq) in DMF (1.47 mL) was added 2-amino-3-(6-methyl-2-oxo-1,2-dihydroquinolin-3-yl)propanamide (64.71 mg, 0.2298 mmol, 1.2 eq), HATU (80.09 mg, 0.2106 mmol, 1.1 eq) and DIPEA (100 μL, 0.5745 mmol, 3.0 eq). The resulting mixture was stirred at rt for overnight. The mixture was directly purified with C-18 reversed-phase column chromatography (mobile phase A: water, mobile phase B: methanol, UV: 214 and 254 nm, Flow rate: 35 mL/min, Gradient: 70-80% (% B)) to give P1.136 (68 mg, 60% yield) as a white solid. ¹H NMR (400 MHz, CD₃OD), δ: 7.78 (s, 1H), 7.75 (s, 1H), 7.37-7.21 (m, 6H), 6.05-6.07 (m, 2H), 4.80-4.47 (m, 2H), 4.64-4.60 (m, 2H), 4.49 (s, 1H), 4.40 (s, 1H), 4.18-3.80 (m, 7H), 3.38-3.34 (m, 1H), 3.20-2.83 (m, 3H), 2.37 (s, 6H), 2.14 (m, 6H), 1.56-1.54 (m, 1H), 1.38-1.35 (m, 1H), 1.01 (s, 18H), 0.89-0.78 (m, 13H).

### Synthesis of (S)-2-amino-3-(6-methyl-2-oxo-1,2-dihydroquinolin-3-yl)propanamide (P1.137)

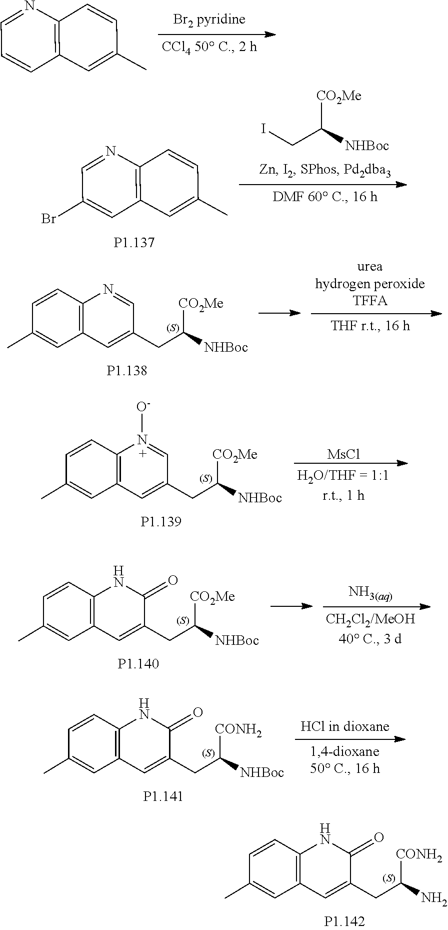

### Preparation 142. 3-Bromo-6-methylquinoline (P1.137)

To a solution of 6-methylquinoline (11.03 g, 77.03 mmol, 1.0 eq) in tetrachloromethane (110 mL) was added bromine (3.95 mL, 77.03 mmol, 1.0 eq) dropwise at rt. The suspension was heated to 50° C., and then pyridine (6.21 mL, 77.03 mmol, 1.0 eq) was added to the reaction mixture at 50° C. dropwise. After the reaction was stirred at 50° C. for 2 h, the reaction was treated with water and extracted with DCM three times. The combined organic layers were dried over Na₂SO₄₍ₛ₎, filtered, and concentrated in vacuo to give the residue, which was purified by silica gel column chromatography (n-hexane:EtOAc=60:1 to 50:1) to give P1.137 (11.4 g, 67% yield) as a white solid. ¹H NMR (400 MHz, CDCl₃), δ: 8.83 (d, J=2.0 Hz, 1H), 8.22 (d, J=2.4 Hz, 1H), 7.97 (d, J=8.6 Hz, 1H), 7.56 (dd, J=8.6, 2.0 Hz, 1H), 7.50 (s, 1H), 2.55 (s, 3H).

### Preparation 143. Methyl (S)-2-((tert-butoxycarbonyl)amino)-3-(6-methylquinolin-3-yl)propanoate (P1.138)

To a mixture of zinc powder (8.84 g, 135.1 mmol, 6.0 eq) in anh. DMF (220 mL) was added iodine (1.71 g, 6.76 mmol, 0.3 eq), and it was stirred at rt. After the color of solution was changed from brown to colorless, added methyl (R)-2-((tert-butoxycarbonyl)amino)-3-iodopropanoate (14.82 g, 45.02 mmol, 2.0 eq) and iodine (1.71 g, 6.76 mmol, 0.3 eq) to the reaction mixture. After the color of solution was changed to colorless, it was stirred at rt for another 30 min. To the reaction mixture was added dicyclohexyl(2′,6′-dimethoxy[1,1′-biphenyl]-2-yl)phosphane (SPhos, 924.2 mg, 2.26 mmol, 0.1 eq) and P1.137 (5.0 g, 22.52 mmol, 1.0 eq), and then degassed by argon for 10 min. To the reaction mixture was added tris(dibenzylideneacetone)dipalladium(0) (Pd₂dba₃, 618.6 mg, 0.676 mmol, 0.03 eq), then stirred at 60° C. for 16 h. The reaction was treated with water and extracted with DCM for three times. The combined organic layers were dried over Na₂SO₄₍ₛ₎, filtered, and concentrated in vacuo to give the residue, which was purified by silica gel column chromatography (n-hexane:EtOAc=5:1 to 2:1) and then C-18 reversed-phase column chromatography (mobile phase A: water (with 0.1% NH₄HCO₃), mobile phase B: methanol, UV: 214 and 254 nm, Flow rate: 80 mL/min, Gradient: 3-90% (% B)) to give P1.138 (4.65 g, 60% yield) as a white foam. ¹H NMR (400 MHz, CDCl₃), δ: 8.62 (s, 1H), 7.96 (d, J=9.2 Hz, 1H), 7.82 (d, J=2.0 Hz, 1H), 7.54-7.50 (m, 2H), 5.08 (d, J=6.8 Hz, 1H), 4.72-4.65 (m, 1H), 3.73 (s, 3H), 3.37-3.18 (m, 2H), 2.54 (s, 3H), 1.42 (s, 9H).

### Preparation 144. (S)-3-(2-((tert-Butoxycarbonyl)amino)-3-methoxy-3-oxopropyl)-6-methylquinoline 1-oxide (P1.139)

To a solution of P1.138 (4.43 g, 12.86 mmol, 1.0 eq) in THF (35 mL) was added urea hydrogen peroxide (4.43 g, 12.86 mmol, 1.0 eq) and trifluoroacetic anhydride (3.58 mL, 25.73 mmol, 2.0 eq). The reaction was stirred at rt for 16 h. The reaction was quenched by slow addition of sat. NaHCO_{3(aq)} and it was extracted with DCM for three times. The combined organic layers were washed by Na₂S₂O_{3(aq)}, dried over Na₂SO₄₍ₛ₎, filtered, and concentrated in vacuo to give P1.139 (4.6 g, crude) as a light-yellow solid, which was used in the next step without further purification. ¹H NMR (400 MHz, CDCl₃), δ: 8.57 (d, J=9.2 Hz, 1H), 8.32 (s, 1H), 7.57-7.52 (m, 2H), 7.44 (s, 1H), 5.14 (d, J=7.6 Hz, 1H), 4.70-4.62 (m, 1H), 3.76 (s, 3H), 3.30-3.20 (m, 1H), 3.17-3.08 (m, 1H), 2.54 (s, 3H), 1.42 (s, 9H).

### Preparation 145. Methyl (S)-2-((tert-butoxycarbonyl)amino)-3-(6-methyl-2-oxo-1,2-dihydroquinolin-3-yl)propanoate (P1.140)

To a solution of P1.139 (4.6 g, 12.87 mmol, 1.0 eq) in water (60 mL) and THF (60 mL) was added methanesulfonyl chloride (1.99 mL, 25.75 mmol, 2.0 eq). The reaction was stirred at rt for 1 h. The reaction was diluted with water and extracted with DCM for three times. The combined organic layers were dried over Na₂SO₄₍ₛ₎, filtered, and concentrated in vacuo to give the residue, which was purified by silica gel column chromatography (n-hexane:EtOAc=2:1 to 1:1) to give P1.140 (3.3 g, 71% yield) as a light-yellow solid. ¹H NMR (400 MHz, CDCl₃), δ: 11.24 (br. s, 1H), 7.62 (s, 1H), 7.34-7.30 (m, 2H), 7.24 (d, J=8.0 Hz, 1H), 6.22 (d, J=6.8 Hz, 1H), 4.60-4.50 (m, 1H), 3.72 (s, 3H), 3.20-3.05 (m, 2H), 2.41 (s, 3H), 1.36 (s, 9H).

### Preparation 146. tert-Butyl (S)-(1-amino-3-(6-methyl-2-oxo-1,2-dihydroquinolin-3-yl)-1-oxopropan-2-yl)carbamate (P1.141)

To a solution of P1.140 (3.01 g, 8.35 mmol, 1.0 eq) in methanol (30 mL) and DCM (30 mL) was added 30% ammonium hydroxide (15 mL). The reaction was stirred at 40° C. for 3 days. The solvent was removed under reduced pressure to give P1.141 (containing an impurity of acid, 3.0 g, crude) as a light-yellow solid which was used in next step without further purification. LCMS (ESI) m/z calc. for C₁₈H₂₃N₃O₄ 345.17, found 246.0 [M-Boc]⁺.

### Preparation 147. (S)-2-Amino-3-(6-methyl-2-oxo-1,2-dihydroquinolin-3-yl)propanamide (P1.142)

To a solution of P1.141 (crude, 8.34 mmol, 1.0 eq) in 1,4-dioxane (58 mL) was added 4M HCl in 1,4-dioxane (10.4 mL, 41.69 mmol, 5.0 eq). The reaction was stirred at 50° C. for 16 h. The reaction mixture was concentrated and purified by C-18 reversed-phase column chromatography (mobile phase A: water (with 0.1% NH₄HCO₃), mobile phase B: methanol, UV: 214 and 254 nm, Flow rate: 60 mL/min, Gradient: 3-90% (% B)). The product was washed with ether to give P1.142 (400 mg, 20% yield) as a light-yellow solid. ¹H NMR (400 MHz, DMSO-d₆), δ: 11.93 (br. s, 1H), 7.86 (s, 1H), 7.74 (s, 1H), 7.51 (s, 1H), 7.42 (s, 1H), 7.34 (dd, J=8.6, 2.0 Hz, 1H), 7.25 (d, J=8.6 Hz, 1H), 4.04-3.97 (m, 1H), 3.05-2.98 (m, 1H), 2.94-2.86 (m, 1H), 2.35 (s, 3H).

### Synthesis of (1R,2S,5S)—N—((S)-1-amino-3-(6-methyl-2-oxo-1,2-dihydroquinolin-3-yl)-1-oxopropan-2-yl)-3-((S)-3,3-dimethyl-2-((5-methylthiazol-2-yl)amino)butanoyl)-6,6-dimethyl-3-azabicyclo[3.1.0]hexane-2-carboxamide (P1.143)

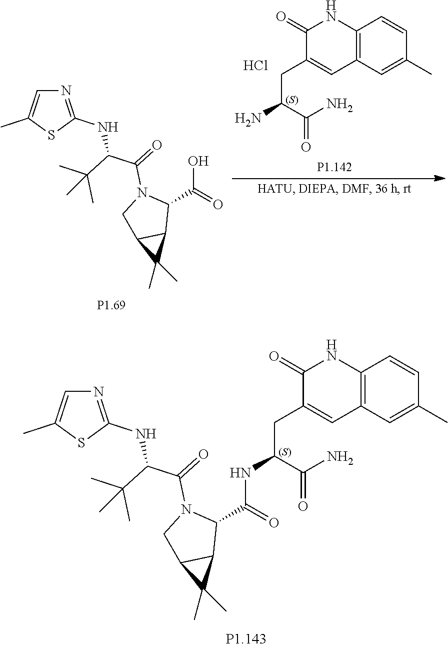

### Preparation 148. (1R,2S,5S)—N—((S)-1-amino-3-(6-methyl-2-oxo-1,2-dihydroquinolin-3-yl)-1-oxopropan-2-yl)-3-((S)-3,3-dimethyl-2-((5-methylthiazol-2-yl)amino)butanoyl)-6,6-dimethyl-3-azabicyclo[3.1.0]hexane-2-carboxamide (P1.143)

To a solution of P1.69 (180 mg, 0.492 mmol) in DMF (3.0 ml) was added P1.142 (167 mg, 0.590 mmol), HATU (393 mg, 1.033 mmol), and DIPEA (428.5 μL, 2.46 mmol). The resulting mixture was stirred at rt overnight. The mixture was purified by silica gel column chromatography by using 0-20% methanol in DCM to obtain desired product P1.143 (53 mg, 18% yield) as a yellow solid. ¹H NMR (400 MHz, CD₃OD), δ: 7.80 (s, 1H), 7.14 (s, 1H), 7.32 (d, J=3.2 Hz 1H), 7.22 (d, J=8.4 Hz 1H), 6.59 (s, 1H), 4.61-4.58 (m, 1H), 4.41 (s, 1H), 4.17 (s, 1H), 4.10 (d, J=3.2 Hz, 1H), 3.14-3.05 (m, 2H), 2.39 (s, 3H), 2.22 (s, 3H), 1.55-1.53 (m, 1H), 1.20 (d, J=7.6 Hz 1H), 1.00 (s, 9H), 0.93 (s, 3H), 0.79 (s, 3H).

### Synthesis of (1R,2S,5S)—N—((S)-1-amino-3-(6-methyl-2-oxo-1,2-dihydroquinolin-3-yl)-1-oxopropan-2-yl)-3-((S)-3,3-dimethyl-2-((2-methylpyrimidin-4-yl)amino)butanoyl)-6,6-dimethyl-3-azabicyclo[3.1.0]hexane-2-carboxamide (P1.144)

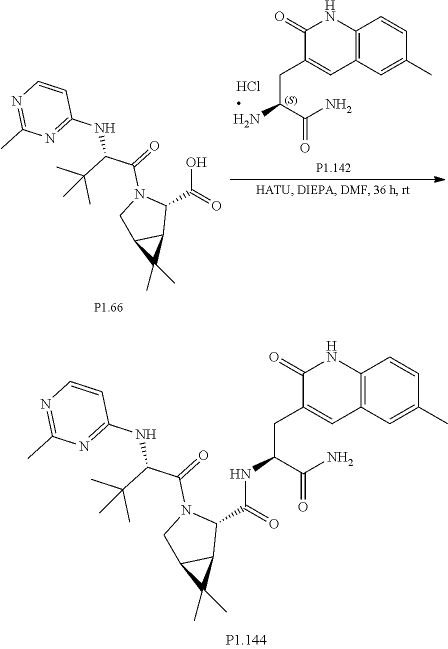

### Preparation 149. (1R,2S,5S)—N—((S)-1-amino-3-(6-methyl-2-oxo-1,2-dihydroquinolin-3-yl)-1-oxopropan-2-yl)-3-((S)-3,3-dimethyl-2-((2-methylpyrimidin-4-yl)amino)butanoyl)-6,6-dimethyl-3-azabicyclo[3.1.0]hexane-2-carboxamide (P1.144)

To a solution of P1.66 (350 mg, 0.971 mmol) in DMF (6.0 mL) was added P1.142 (410 mg, 2.913 mmol), HATU (738 mg, 2.913 mmol) and DIPEA (1.02 mL, 7.768 mmol). The resulting mixture was stirred at rt for 36 h. Then reaction mixture was treated with ethyl acetate and washed with water. The organic layer was dried over Na₂SO₄₍ₛ₎ filtered, and concentrated. The crude was firstly purified by silica gel column chromatography (0-20% methanol in DCM) and further purification by using C18 reverse phase column (0-80% MeOH in water) to give desired product P1.144 (109 mg, 22% yield) as a yellow solid. ¹H NMR (400 MHz, CD₃OD), δ: 7.86 (d, J=6.2 Hz 1H), 7.80 (s, 1H), 7.40 (s, 1H), 7.32 (d, J=8.8 Hz, 1H), 7.21 (d, J=8.0 Hz, 1H), 6.44 (d, J=6.2 Hz, 1H), 4.97 (br. s, 1H), 4.60-4.57 (m, 1H), 4.25-4.13 (m, 3H), 3.15-3.00 (m, 1H), 2.41 (s, 3H), 2.37 (s, 3H), 1.58-1.54 (m, 1H), 1.23 (d, J=7.6 Hz, 1H), 1.00 (s, 9H), 0.90 (s, 3H), 0.71 (s, 3H). LRMS (ESI) m/z 588.1 [M+H]⁺.

### Synthesis of (1R,2S,5S)—N—((S)-1-amino-3-(6-methyl-2-oxo-1,2-dihydroquinolin-3-yl)-1-oxopropan-2-yl)-3-((S)-3,3-dimethyl-2-((4-methylpyrimidin-2-yl)amino)butanoyl)-6,6-dimethyl-3-azabicyclo[3.1.0]hexane-2-carboxamide (P1.147)

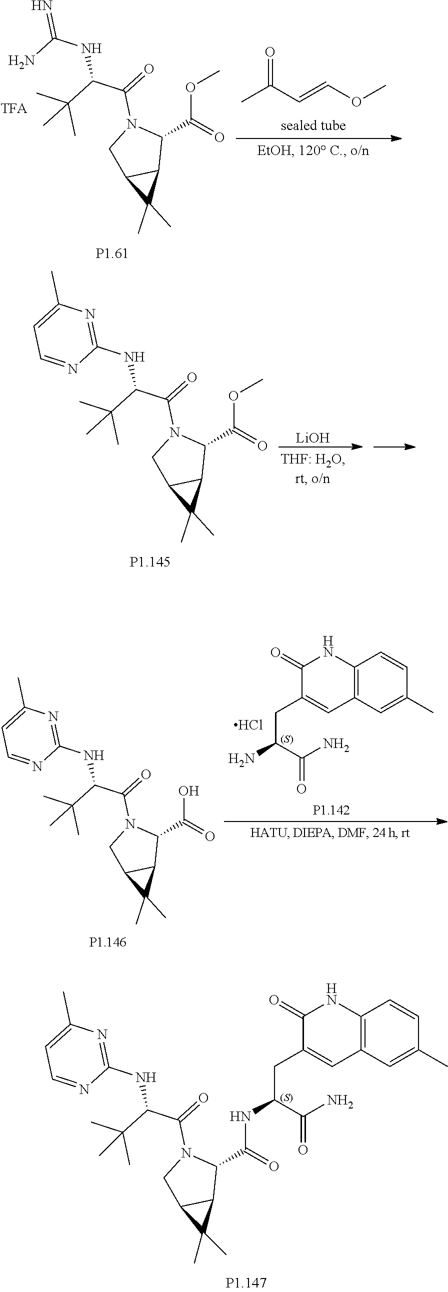

### Preparation 150. Methyl (1R,2S,5S)-3-((S)-3,3-dimethyl-2-((4-methylpyrimidin-2-yl)amino)butanoyl)-6,6-dimethyl-3-azabicyclo[3.1.0]hexane-2-carboxylate (P1.145)

To a solution of P1.61 (1.59 g, 3.77 mmol) in ethanol (48 mL) was added (E)-4-methoxybut-3-en-2-one at rt (1.92 mL, 18.86 mmol). The reaction was stirred overnight in a sealed tube at 120° C. The solvents were evaporated under reduced pressure and crude reaction mixture was purified by silica gel column chromatography (0-30% ethyl acetate in hexane) to give P1.145 (270 mg, 19%) as a brown solid. ¹H NMR (400 MHz, CD₃OD), δ: 8.11 (d, J=5.0 Hz 1H), 6.56 (d, J=5.0 Hz, 1H), 4.59 (br. s, 1H), 4.34 (s, 1H), 4.27 (d, J=10.4 Hz, 1H), 4.04-4.00 (m, 1H), 3.74 (s, 3H), 2.32 (s, 3H), 1.66-1.60 (m, 1H), 1.47 (d, J=7.6 Hz, 1H), 1.11-1.06 (m, 12H), 0.86 (s, 3H).

### Preparation 151. (1R,2S,5S)-3-((S)-3,3-Dimethyl-2-((4-methylpyrimidin-2-yl)amino)butanoyl)-6,6-dimethyl-3-azabicyclo[3.1.0]hexane-2-carboxylic acid (P1.146)

To a solution of P1.145 (400 mg 1.07 mmol) in THF (4.0 mL) was added LiOH (22.6 mg, 0.945 mmol) solution in water (4.0 mL). The resulting mixture was stirred at rt for overnight. The solvents were evaporated to obtain brown syrup P1.146 (525 mg, quantitative yield) as a crude product, which was directly used in next step without further purification. LCMS (ESI) m/z 361.2 [M+H]⁺.

### Preparation 152. (1R,2S,5S)—N—((S)-1-Amino-3-(6-methyl-2-oxo-1,2-dihydroquinolin-3-yl)-1-oxopropan-2-yl)-3-((S)-3,3-dimethyl-2-((4-methylpyrimidin-2-yl)amino)butanoyl)-6,6-dimethyl-3-azabicyclo[3.1.0]hexane-2-carboxamide (P1.147)

To a solution of P1.146 (300 mg, 0.832 mmol) in DMF (6.0 mL) was added chiral 2-amino-3-(6-methyl-2-oxo-1,2-dihydroquinolin-3-yl)propenamide (281 mg, 0.998 mmol), HATU (664 mg, 1.75 mmol) and DIPEA (724 μL, 4.16 mmol). The resulting mixture was stirred at rt for overnight. The mixture was purified by silica gel column chromatography (0-20% methanol in DCM) to obtain a desired product P1.147 (170 mg, 35% yield) as a yellow solid. ¹H NMR (400 MHz, CD₃OD), δ: 8.10 (d, J=5.2 Hz 1H), 7.82 (s, 1H), 7.44 (s, 1H), 7.35-7.33 (m, 1H), 7.22 (d, J=8.4 Hz 1H), 6.55-6.54 (d, J=5.2 Hz, 1H), 4.62-4.59 (m, 1H), 4.22-4.12 (m, 3H), 3.18-3.13 (m, 1H), 3.08-3.02 (m, 1H), 2.40 (s, 3H), 2.31 (s, 3H), 1.60-1.56 (m, 1H), 1.25 (d, J=7.6 Hz 1H), 1.00 (s, 9H), 0.93 (s, 3H), 0.78 (s, 3H).

### Synthesis of 4-(methylsulfonyl)-2-(trifluoromethyl)pyrimidine (P1.150)

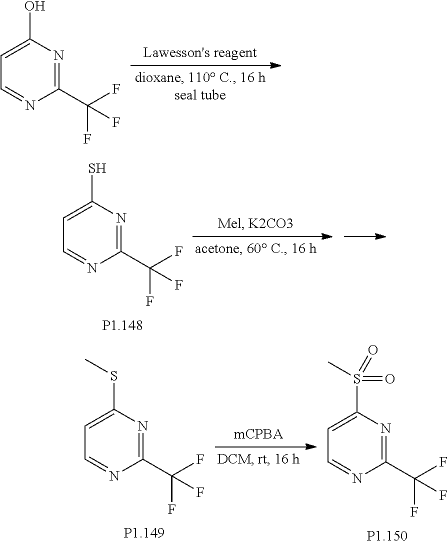

### Preparation 153. 2-(Trifluoromethyl)pyrimidine-4-thiol (P1.148)

To a solution of 4-hydroxy-2-(trifluoromethyl)pyrimidine (1.75 g, 10.66 mmol) in 1,4-dioxane (11 mL) was added Lawesson's reagent (216 mg, 5.33 mmol). After the reaction was stirred at 110° C. for 16 h in a sealed tube, the solvent was evaporated (kept a little solvent inside). The reaction mixture was purified by silica gel column chromatography (0-50% ethyl acetate in n-hexane). The fractions of the desired product were collected and treated with DCM and diethyl ether to concentrate under reduced pressure to give a desired product P1.148 (2 g, containing solvents) as a yellow liquid. ¹H NMR (400 MHz, CDCl₃), δ: 8.47 (d, J=5.6 Hz, 1H), 7.34 (d, J=5.6 Hz, 1H).

### Preparation 154. 4-(Methylthio)-2-(trifluoromethyl)pyrimidine (P1.149)

To a solution of P1.148 (2 g, 11 mmol, crude) in acetone (67 mL) was added potassium carbonate (1.77 g, 12.8 mmol) and methyl iodide (0.7 mL, 10.66 mmol). After the reaction was stirred at 60° C. for 16 h, the reaction mixture was concentrated, treated with water, and extracted with ethyl acetate. The combined organic layers were dried over Na₂SO₄₍ₛ₎ filtered, and concentrated to give P1.149 (1.7 g, 78% yield) as a yellow oil. ¹H NMR (400 MHz, CDCl₃), δ: 8.43 (d, J=5.6 Hz, 1H), 7.29 (d, J=5.6 Hz, 1H), 2.60 (s, 3H).

### Preparation 155. 4-(Methylsulfonyl)-2-(trifluoromethyl)pyrimidine (P1.150)

A solution of P1.149 (1.7 g, 11 mmol, crude) and 3-chloroperbenzoic acid (5.5 g, 31.98 mmol) in DCM (107 mL) was stirred at rt for 16 h. The reaction mixture was treated with NaHCO_{3(aq)} and extracted with DCM. The combined organic layers were dried over Na₂SO₄₍ₛ₎, filtered, and concentrated. The residue was purified by silica gel column chromatography (0-40% ethyl acetate in n-hexane) to give desired product P1.150 (1.44 g, 72% yield) as a yellow solid. ¹H NMR (400 MHz, CDCl₃), δ: 9.29 (d, J=5.0 Hz, 1H), 8.20 (d, J=5.0 Hz, 1H), 3.36 (s, 3H).

### Synthesis of (1R,2S,5S)—N—((S)-1-amino-3-(6-methyl-2-oxo-1,2-dihydroquinolin-3-yl)-1-oxopropan-2-yl)-3-((S)-3,3-dimethyl-2-((2-(trifluoromethyl)pyrimidin-4-yl)amino)butanoyl)-6,6-dimethyl-3-azabicyclo[3.1.0]hexane-2-carboxamide (P1.153)

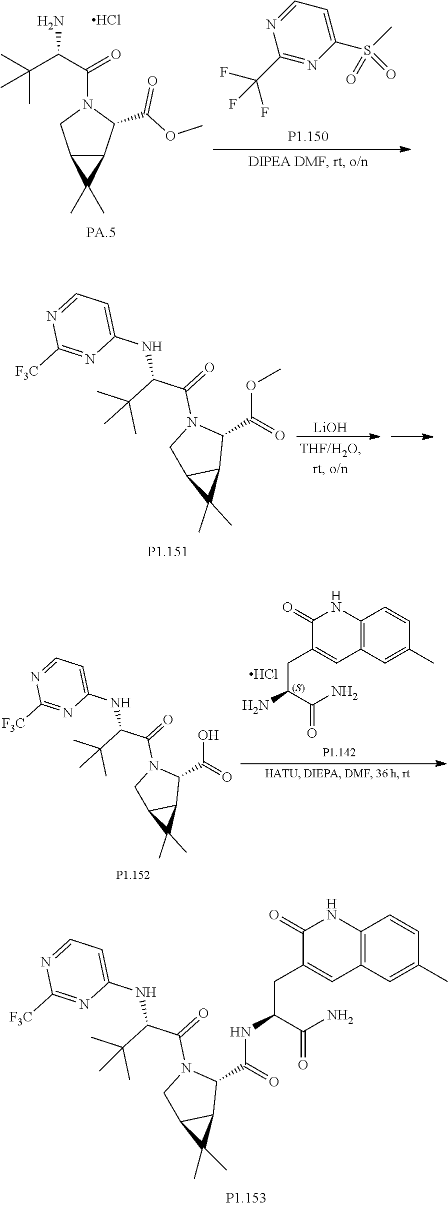

### Preparation 156. Methyl (1R,2S,5S)-3-((S)-3,3-dimethyl-2-((2-(trifluoromethyl)pyrimidin-4-yl)amino)butanoyl)-6,6-dimethyl-3-azabicyclo[3.1.0]hexane-2-carboxylate (P1.151)

To a solution of PA.1 (338 mg, 319 mmol) and N,N-diisopropylethylamine (0.462 mL, 2.65 mmol) in anh. DMF (1.8 mL) was added P1.150 (200 mg, 0.884 mmol). The reaction was stirred at rt for overnight. The reaction mixture was treated with water and extracted with ethyl acetate. The combined organic layers were dried over Na₂SO₄₍ₛ₎ filtered, and concentrated. The residue was purified by silica gel column chromatography (0-30% ethyl acetate in n-hexane) to give P1.151 (200 mg, 44% yield) as a yellow oil. ¹H NMR (400 MHz, CDCl₃), δ: 8.14 (d, J=6.0 Hz, 1H), 6.42 (d, J=6.0 Hz, 1H), 5.82 (d, J=9.0 Hz, 1H), 5.00 (d, J=9.0 Hz, 1H), 4.11-4.07 (m, 1H), 3.98-3.94 (m, 1H), 3.74 (s, 3H), 1.51-1.48 (m, 1H), 1.42 (d, J=7.6 Hz, 1H), 1.07 (s, 9H), 1.01 (s, 3H), 0.70 (s, 3H).

### Preparation 157. (1R,2S,5S)-3-((S)-3,3-Dimethyl-2-((2-(trifluoromethyl)pyrimidin-4-yl)amino)butanoyl)-6,6-dimethyl-3-azabicyclo[3.1.0]hexane-2-carboxylic acid (P1.152)

A solution of P1.151 (200 mg, 0.467 mmol) in THF (2.0 mL) was added to a solution of LiOH (34.0 mg, 1.401 mmol) in water (2.0 mL). After the resulting mixture was stirred at rt for overnight. The solvents were evaporated to obtain a brown solid of P1.152 (191 mg, quantitative yield) as a crude product which was used in next step without further purification. LCMS (ESI) m/z 415.2 [M+H]⁺.

### Preparation 158. (1R,2S,5S)—N—((S)-1-Amino-3-(6-methyl-2-oxo-1,2-dihydroquinolin-3-yl)-1-oxopropan-2-yl)-3-((S)-3,3-dimethyl-2-((2-(trifluoromethyl)pyrimidin-4-yl)amino)butanoyl)-6,6-dimethyl-3-azabicyclo[3.1.0]hexane-2-carboxamide (P1.153)

To a solution of P1.152 (191 mg, 0.460 mmol) in DMF (3.0 mL) was added P1.142 (390 mg, 0.510 mmol), HATU (525 mg, 1.38 mmol), and DIPEA (1.28 mL, 4.14 mmol). The mixture was stirred at rt for 36 h. The solution was treated with water and extracted with ethyl acetate. The combined organic layers were dried over Na₂SO₄₍ₛ₎ filtered, and concentrated. The residue was purified by silica gel column chromatography (0-10% DCM in methanol) to give desired product P1.153 (65 mg, 22% yield) as a yellow solid. ¹H NMR (400 MHz, CD₃OD), δ: 8.12 (d, J=6.0 Hz 1H), 7.80 (s, 1H), 7.40-7.20 (m, 3H), 6.78 (d, J=6.0 Hz, 1H), 5.05 (s, 1H), 4.65-4.61 (m, 1H), 4.19-4.16 (m, 3H), 3.14-3.06 (m, 2H), 2.38 (s, 3H), 1.57-1.56 (m, 1H), 1.24 (d, J=7.6 Hz 1H)), 1.02 (s, 9H), 0.90 (s, 3H), 0.65 (s, 3H).

### Synthesis of(S)-3,3-dimethyl-2-((2,2,2-trifluoroethyl)sulfonamido)butanoic acid (P1.155)

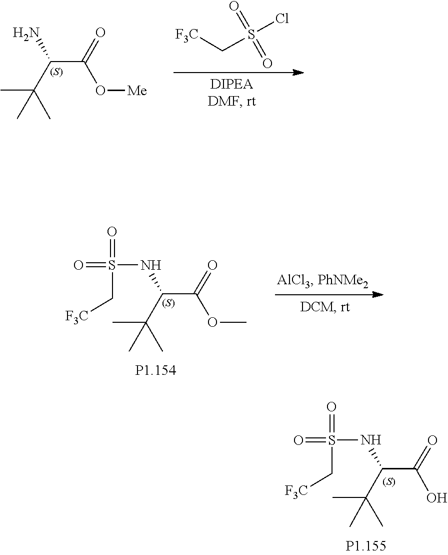

### Preparation 159. (S)-3,3-Dimethyl-2-((2,2,2-trifluoroethyl)sulfonamido)butanoate (P1.154)

To a solution of methyl (2S)-2-amino-3,3-dimethyl-butanoate (1.00 g, 5.50 mmol) and N,N-diisopropylethylamine (2.88 mL, 26.5 mmol) in anh. DMF (25 mL) was added 2,2,2-trifluoroethane-1-sulfonyl chloride (1.33 mL, 11.0 mmol). After the reaction was stirred at rt for 16 h, the mixture was diluted with EtOAc and washed with water and NaHCO_{3(aq)}. The organic layer was dried over MgSO₄, filtered, and concentrated under reduced pressure. The residue was purified by silica gel column chromatography (0-50% EtOAc in n-hexane) to give P1.154 (1.65 g, 99%) as a colorless gum. ¹H NMR (400 MHz, DMSO-d₆), δ: 8.25 (d, J=9.2 Hz, 1H), 4.45-4.23 (m, 2H), 3.80 (d, J=9.2 Hz, 1H), 3.65 (s, 3H), 0.94 (s, 9H); LCMS (ESI) m/z calc. for C₉H₁₆F₃NO₄S 291.08. found, 289.8 [M−H]⁺.

### Preparation 160. (S)-3,3-Dimethyl-2-((2,2,2-trifluoroethyl)sulfonamido)butanoic acid (P1.155)

To a solution of P1.154 (1.60 g, 5.49 mmol) in DCM (35 mL) and N,N-dimethylaniline (3.48 mL, 27.5 mmol) was added aluminum chloride (2.20 g, 16.5 mmol). After the reaction was stirred at rt for 16 h, the mixture was diluted with diethyl ether and quenched with water. The organic layer was washed with 1N HCl_{(aq)}, dried over MgSO₄, filtered, and concentrated under reduced pressure to obtain P1.155 (1.36 g, crude) as a green gum which was used in the next step without further purification. ¹H NMR (400 MHz, DMSO-d₆), δ: 8.06 (d, J=9.4 Hz, 1H), 4.42-4.18 (m, 2H), 3.67 (d, J=9.4 Hz, 1H), 0.96 (s, 9H); LCMS (ESI) m/z calc. for C₈H₁₄F₃NO₄S 277.06. found, 275.8 [M−H]⁺.

### Synthesis of (2S)—N-((2S)-1-((1-amino-3-(6-methyl-2-oxo-1,2-dihydroquinolin-3-yl)-1-oxopropan-2-yl)amino)-1-oxo-3-phenylpropan-2-yl)-3,3-dimethyl-2-((2,2,2-trifluoroethyl)sulfonamido)butanamide (P1.158)

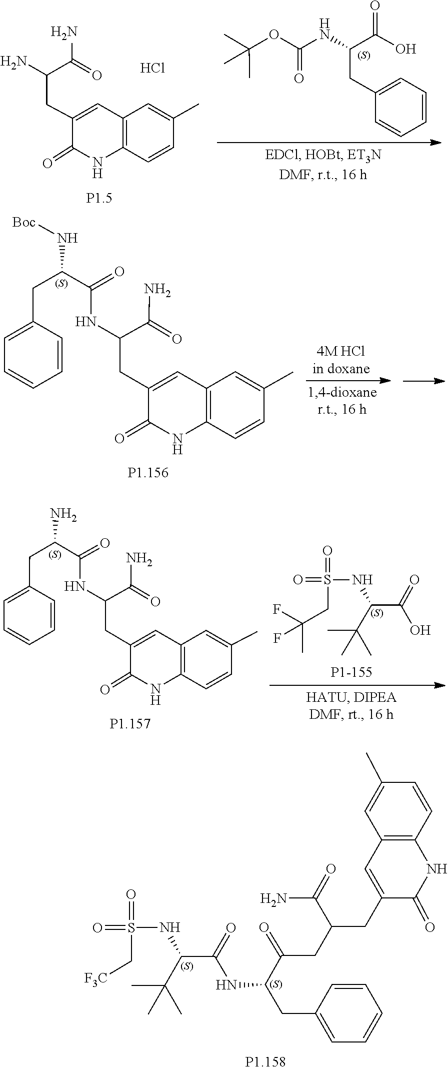

### Preparation 161. tert-Butyl ((2S)-1-((1-amino-3-(6-methyl-2-oxo-1,2-dihydroquinolin-3-yl)-1-oxopropan-2-yl)amino)-1-oxo-3-phenylpropan-2-yl)carbamate (P1.156)

To a solution of P1.5 (HCl salt, 300 mg, 1.06 mmol, 1.0 eq) in N,N-dimethylformamide (5 mL) was added (tert-butoxycarbonyl)-L-phenylalanine (282 mg, 1.06 mmol, 1.0 eq), 1-(3-dimethylaminopropyl)-3-ethylcarbodiimide hydrochloride (245 mg, 1.27 mmol, 1.2 eq), and 1-hydroxybenzotriazole hydrate (195 mg, 1.27 mmol, 1.2 eq) at rt. After the reaction solution was stirred at rt for 20 min, triethylamine (0.593 mL, 4.25 mmol, 4.0 eq) was added and the solution was stirred at rt for 16 h. The reaction solution was concentrated under reduced pressure and purified by silica gel chromatography (10% methanol in dichloromethane). The solid was suspended in methanol, filtered and washed with methanol to afford P1.156 as a yellow solid. ¹H NMR (400 MHz, DMSO-d₆), δ: 11.70-11.69 (m, 2H), 8.33 (d, J=8.4 Hz, 1H), 8.11 (d, J=8.0 Hz, 1H), 7.63 (s, 2H), 7.36-6.90 (m, 22H), 4.50-4.47 (m, 2H), 4.07-4.04 (m, 2H), 3.04-2.53 (m, 6H), 2.30 (s, 3H), 2.28 (s, 3H), 1.27 (s, 9H), 1.25 (s, 9H).

### Preparation 162. (2S)-2-Amino-N-(1-amino-3-(6-methyl-2-oxo-1,2-dihydroquinolin-3-yl)-1-oxopropan-2-yl)-3-phenylpropanamide (P1.157)

To a solution of P1.156 (414 mg, 0.840 mmol, 1.0 eq) in 1,4-dioxane (8.4 mL) was added 4 M hydrogen chloride in dioxane (0.840 mL, 3.36 mmol, 4.0 eq) and stirred at rt for 16 h. The precipitate was collected by filtration and washed with 1,4-dioxane. The precipitate was dried under high vacuum to afford P1.157 (HCl salt, 357 mg, 99%) as a white solid. ¹H NMR (400 MHz, DMSO-d₆), δ: 11.75 (br. s, 2H), 8.97-8.94 (m, 2H), 8.20 (br. s, 3H), 8.11 (br. s, 3H), 7.70 (s, 2H), 7.52-7.03 (m, 22H), 4.58-4.55 (m, 2H), 4.05-4.00 (m, 2H), 3.15-2.81 (m, 6H), 2.68-2.59 (m, 2H), 2.32 (s, 3H), 2.26 (s, 3H).

### Preparation 163. (2S)—N-((2S)-1-((1-Amino-3-(6-methyl-2-oxo-1,2-dihydroquinolin-3-yl)-1-oxopropan-2-yl)amino)-1-oxo-3-phenylpropan-2-yl)-3,3-dimethyl-2-((2,2,2-trifluoroethyl)sulfonamido)butanamide (P1.158)

To a solution of P1.157 (HCl salt, 100 mg, 0.233 mmol, 1.0 eq) in DMF (2.5 ml) was added P1.155 (77.5 mg, 0.279 mmol, 1.2 eq), HATU (97.4 mg, 0.256 mmol, 1.1 eq) and DIPEA (0.121 ml, 0.699 mmol, 3.0 eq) at rt. After the reaction mixture was stirred at rt for 16 h, the solution was purified by C18 flash column (5-100% methanol in H₂O) to afford P1.158 (93 mg, 61%) as a white solid. ¹H NMR (400 MHz, DMSO-d₆), δ: 11.71-11.69 (m, 2H), 8.43-8.35 (m, 2H), 8.21-8.16 (m, 2H), 7.82-7.73 (m, 2H), 7.62-7.59 (m, 2H), 7.31-7.01 (m, 22H), 4.58-4.47 (m, 4H), 3.78-3.54 (m, 6H), 3.04-2.89 (m, 4H), 2.78-2.73 (m, 2H), 2.62-2.51 (m, 2H), 2.30 (s, 3H), 2.28 (s, 3H), 0.81 (s, 9H), 0.73 (m, 9H).

### Synthesis of (S)-2-((tert-butoxycarbonyl)amino)-3-(2,4,5-trifluorophenyl)propanoic acid (P1.160)

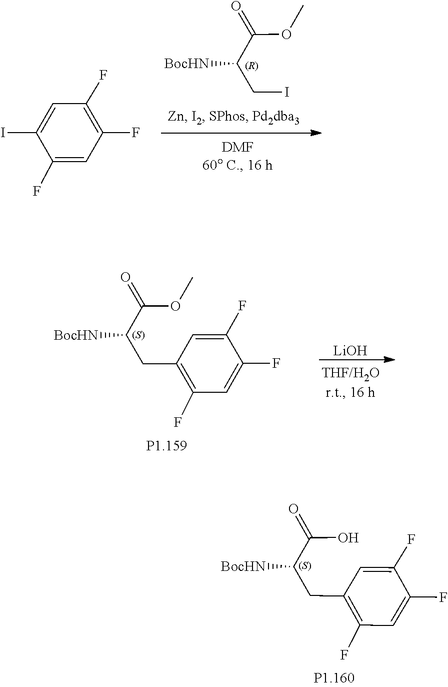

### Preparation 164. Methyl (S)-2-((tert-butoxycarbonyl)amino)-3-(2,4,5-trifluorophenyl)propanoate (P1.159)

To a mixture of zinc powder (655.5 mg, 10.03 mmol, 3.0 eq) in anh. DMF (35 mL) was added iodine (127.2 mg, 0.501 mmol, 0.15 eq), and it was stirred at rt. After the color of solution was changed from brown to colorless, methyl (R)-2-((tert-butoxycarbonyl)amino)-3-iodopropanoate (1.10 g, 3.34 mmol, 1.0 eq) and iodine (127.2 mg, 0.501 mmol, 0.15 eq) was added to the reaction mixture. After the color of solution was changed to colorless again, the solution was stirred at rt for another 30 min. To the reaction mixture was added dicyclohexyl(2′,6′-dimethoxy[1,1′-biphenyl]-2-yl)phosphane (SPhos, 137.2 mg, 0.334 mmol, 0.1 eq) and 1,2,4-trifluoro-5-iodo-benzene (0.5 mL, 4.01 mmol, 1.2 eq), and then degassed by argon for 10 min. To the reaction mixture was added tris(dibenzylideneacetone)dipalladium(0) (91.8 mg, 0.10 mmol, 0.03 eq). The reaction was stirred at 60° C. for 16 h. The reaction was treated with water and extracted with ethyl acetate for three times. The combined organic layers were dried over Na₂SO₄₍ₛ₎, filtered, and concentrated in vacuo to give the residue, which was purified by silica gel column chromatography (n-hexane:EtOAc=20:1 to 10:1) to give P1.159 (794 mg, 72% yield) as a light-yellow solid. ¹H NMR (400 MHz, CDCl₃), δ: 7.03-6.85 (m, 2H), 5.07 (d, J=6.8 Hz, 1H), 4.54 (q, J=6.8 Hz, 1H), 3.75 (s, 3H), 3.19-3.15 (m, 1H), 3.03-2.95 (m, 1H), 1.41 (s, 9H).

### Preparation 165. (S)-2-((tert-Butoxycarbonyl)amino)-3-(2,4,5-trifluorophenyl)propanoic acid (P1.160)

To a solution of P1.159 (794 mg, 2.38 mmol, 1.0 eq) in THF (10 mL) and water (10 mL) was added lithium hydroxide (171.1 mg, 7.15 mmol, 3.0 eq). The reaction was stirred at rt for 16 h. The mixture was acidified by slow addition of 1 N HCl_{(aq)} and adjusted pH value to 6. The reaction mixture was extracted with DCM three times. The combined organic layers were dried over Na₂SO₄₍ₛ₎, filtered, and concentrated in vacuo to give crude P1.160 (723 mg, 95% yield) as a light yellow solid. ¹H NMR (400 MHz, CDCl₃), δ: 7.08-6.99 (m, 1H), 6.96-6.87 (m, 1H), 5.05 (d, J=8.0 Hz, 1H), 4.63-4.39 (m, 1H), 3.25 (dd, J=14.0, 5.6 Hz, 1H), 3.06-2.84 (m, 1H), 1.41 (s, 9H).

### Synthesis of (2S)—N-((2S)-1-((1-Amino-3-(6-methyl-2-oxo-1,2-dihydroquinolin-3-yl)-1-oxopropan-2-yl)amino)-1-oxo-3-(2,4,5-trifluorophenyl)propan-2-yl)-3,3-dimethyl-2-((2,2,2-trifluoroethyl)sulfonamido)butanamide (P1.163)

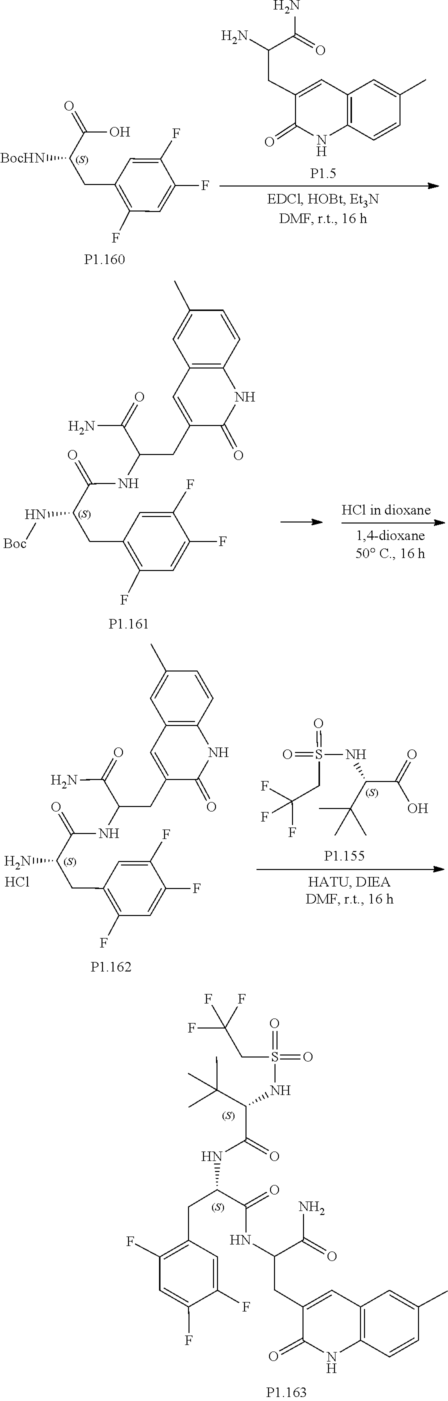

### Preparation 166. tert-Butyl ((2S)-1-((1-amino-3-(6-methyl-2-oxo-1,2-dihydroquinolin-3-yl)-1-oxopropan-2-yl)amino)-1-oxo-3-(2,4,5-trifluorophenyl)propan-2-yl)carbamate (P1.161)

To a solution of P1.160 (100.0 mg, 0.313 mmol, 1.0 eq) and P1.5 (88.2 mg, 0.313 mmol, 1.0 eq) in DMF (1.6 mL) was added EDCI×HCl (72.1 mg, 0.376 mmol, 1.2 eq) and HOBt (57.6 mg, 0.376 mmol, 1.2 eq). After the reaction mixture was stirred at rt for 20 min, triethylamine (0.17 mL, 1.25 mmol, 4.0 eq) was added to the mixture, and it was stirred at rt for another 16 h. The mixture was concentrated and purified by silica gel column chromatography (DCM:MeOH=20:1 to 15:1) to give P1.161 (55 mg, 32% yield) as a yellow solid. ¹H NMR (400 MHz, DMSO-d₆), δ: 11.72 (s, 1H), 11.70 (s, 1H), 8.38-8.35 (d, J=8.0 Hz, 1H), 8.14 (d, J=8.4 Hz, 1H), 7.63-7.60 (m, 2H), 7.39-6.98 (m, 14H), 4.55-4.41 (m, 2H), 4.20-4.05 (m, 2H), 3.05-2.84 (m, 4H), 2.81-2.56 (m, 4H), 2.31 (s, 3H), 2.98 (s, 3H), 1.27 (s, 9H), 1.25 (s, 9H).

### Preparation 167. (2S)-2-Amino-N-(1-amino-3-(6-methyl-2-oxo-1,2-dihydroquinolin-3-yl)-1-oxopropan-2-yl)-3-(2,4,5-trifluorophenyl)propanamide (P1.162)

To a mixture of P1.161 (55.0 mg, 0.101 mmol, 1.0 eq) in 1,4-dioxane (1.1 mL) was added 4 M HCl in 1,4-dioxane solution (0.13 mL, 0.503 mmol, 5.0 eq). The reaction was stirred at 50° C. for 16 h. The solvent was removed under reduced pressure to give P1.162 (52 mg, crude) as a light yellow solid which was used in next step without further purification. ¹H NMR (400 MHz, DMSO-d₆), δ: 11.76 (s, 1H), 11.74 (s, 1H), 8.89 (d, J=8.4 Hz, 1H), 8.80 (d, J=7.2 Hz, 1H), 8.24 (br. s, 3H) 8.16 (s, 3H), 7.66 (s, 1H), 7.64 (s, 1H), 7.57-7.09 (m, 14H), 4.66-4.52 (m, 2H), 4.14-3.98 (m, 2H), 3.04-2.80 (m, 6H), 2.72-2.63 (m, 2H), 2.33 (s, 3H), 2.28 (s, 1H).

### Preparation 168. (2S)—N-((2S)-1-((1-Amino-3-(6-methyl-2-oxo-1,2-dihydroquinolin-3-yl)-1-oxopropan-2-yl)amino)-1-oxo-3-(2,4,5-trifluorophenyl)propan-2-yl)-3,3-dimethyl-2-((2,2,2-trifluoroethyl)sulfonamido)butanamide (P1.163)

To a solution of P1.162 (52.0 mg, 0.108 mmol, 1.0 eq) and P1.155 (35.8 mg, 0.129 mmol, 1.2 eq) in DMF (1.0 mL) was added HATU (45.0 mg, 0.118 mmol, 1.1 eq) and DIPEA (0.06 mL, 0.323 mmol, 3.0 eq). The reaction mixture was stirred at rt for 16 h. The solution was concentrated and purified by C-18 reversed-phase column chromatography (mobile phase A: water (with 0.1% NH₄HCO₃), mobile phase B: methanol, UV: 214 and 254 nm, Flow rate: 35 mL/min, Gradient: 3-90% (% B)) to give P1.163 (40 mg, 53% yield) as a light-yellow solid. ¹H NMR (400 MHz, DMSO-d₆), δ: 11.73 (s, 1H), 11.70 (s, 1H), 8.42-8.23 (m, 4H), 7.84 (d, J=8.8 Hz, 1H), 7.75 (d, J=8.4 Hz, 1H), 7.60 (s, 1H), 7.59 (s, 1H), 7.44-7.04 (m, 14H), 4.69-4.59 (m, 2H), 4.55-4.47 (m, 2H), 4.04-3.69 (m, 4H), 3.67-3.60 (m, 2H), 3.04-2.73 (m, 6H), 2.69-2.56 (m, 2H), 2.31 (s, 3H), 2.29 (s, 3H), 0.85 (s, 9H), 0.72 (s, 9H).

### Synthesis of (S)-3-(6-methyl-2-oxo-1,2-dihydroquinolin-3-yl)-2-(methylamino)propanamide (P1.168)

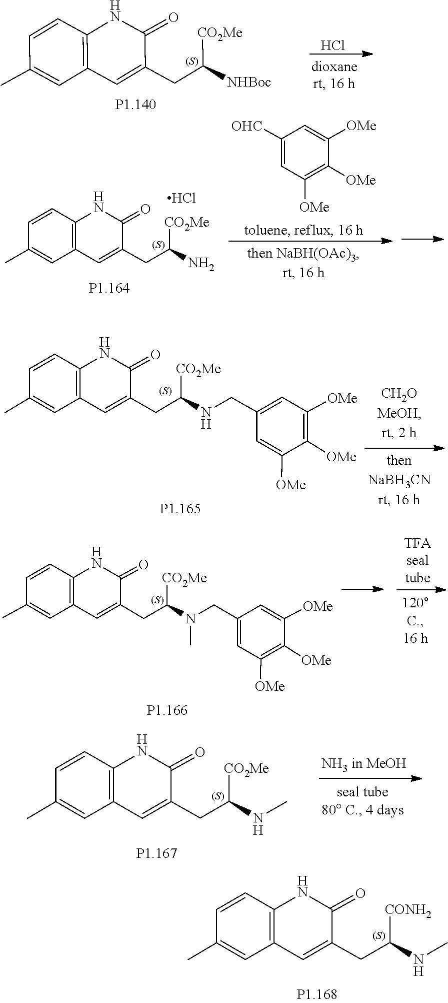

### Preparation 169. (S)-2-Amino-3-(6-methyl-2-oxo-1,2-dihydroquinolin-3-yl)propanoate hydrochloride (P1.164)

To a solution of P1.140 (3.0 g, 8.4 mmol) in dioxane (45 mL) was added 4 M HCl in dioxane (10 mL, 40 mmol). After the reaction mixture was stirred at 50° C. for 16 h, the mixture was concentrated under reduced pressure to give P1.164 (2.47 g, 99%) as a pink solid which was used in next with further purification. ¹H NMR (400 MHz, CD₃OD), δ: 7.88 (s, 1H), 7.48 (s, 1H), 7.42 (d, J=8.4 Hz, 1H), 7.28 (d, J=8.4 Hz, 1H), 4.45-4.42 (m, 1H), 3.78 (s, 3H), 3.29-3.23 (m, 1H), 3.20-3.09 (m, 1H), 2.42 (s, 3H); LCMS (ESI): 261.1 [M+H]⁺.

### Preparation 170. Methyl (S)-3-(6-methyl-2-oxo-1,2-dihydroquinolin-3-yl)-2-((3,4,5-trimethoxybenzyl)amino)propanoate (P1.165)

A solution of P1.164 (2.13 g, 7.18 mmol), 3,4,5-trimethoxybenzaldehyde (1.69 g, 8.61 mmol), NaOAc (1.77 g, 21.5 mmol) and Na₂SO₄₍ₛ₎ (1.00 g, 7.18 mmol) in anh. toluene (70 mL) was refluxed with Dean-Stark apparatus for 16 h. The mixture was cooled down to rt, followed by NaBH(OAc)₃ (3.04 g, 14.4 mmol) was added. After the reaction mixture was stirred at 40° C. for 16 h, The solution was quenched with water and extracted with EtOAc. The combined organic layers were washed with H₂O, dried over MgSO₄, filtered, and concentrated under reduced pressure. The residue was purified by reverse phase chromatography (10-100% MeOH in H₂O) to give P1.165 (1.14 g, 36%) as a white solid. ¹H NMR (400 MHz, CDCl₃), δ: 10.98 (br. s, 1H), 7.58 (s, 1H), 7.30-7.26 (m, 2H), 7.17 (d, J=8.8 Hz, 1H), 6.50 (s, 2H), 3.82-3.76 (m, 5H), 3.72 (s, 6H), 3.69 (s, 3H), 3.62-3.59 (m, 1H), 3.08-2.95 (m, 2H), 2.40 (s, 3H); LCMS (ESI): 441.5 [M+H]⁺.

### Preparation 170. Methyl (S)-2-(methyl(3,4,5-trimethoxybenzyl)amino)-3-(6-methyl-2-oxo-1,2-dihydroquinolin-3-yl)propanoate (P1.166)

To a stirred solution of P1.165 (1.14 g, 2.59 mmol) in MeOH (15 mL) was added formaldehyde (37 wt. % in H₂O, 0.19 mL, 2.6 mmol) at rt. After 1 h, NaBH₃CN (163 mg, 2.59 mmol) was added to the mixture. After the reaction mixture was stirred at rt for 16 h, the mixture was extracted with DCM. The combined organic layers were washed with NaHCO_{3(aq)} and brine, dried over MgSO₄, filtered, and concentrated under reduced pressure. The residue was purified by silica gel chromatography (0-5% MeOH in DCM) to give P1.166 (767 mg, 65%) as a white solid. ¹H NMR (400 MHz, CDCl₃), δ: 10.74 (br. s, 1H), 7.57 (s, 1H), 7.28 (s, 1H), 7.15 (d, J=8.8 Hz, 1H), 6.38 (s, 2H), 4.12-4.08 (m, 1H), 3.87-3.78 (m, 2H), 3.76 (s, 3H), 3.75 (s, 3H), 3.64-3.51 (m, 7H), 3.20-3.01 (m, 2H), 2.39 (s, 3H), 2.29 (s, 3H); LCMS (ESI): 455.1 [M+H]⁺.

### Preparation 171. Methyl (S)-3-(6-methyl-2-oxo-1,2-dihydroquinolin-3-yl)-2-(methylamino)propanoate (P1.167)

A solution of P1.166 (550 mg, 1.21 mmol) in anh. TFA (12 mL) in a sealed tube was heated at 120° C. for 16 h. After completion, the reaction mixture was concentrated under reduced pressure. The crude was diluted with MeOH and then neutralized by 7N NH₃ in MeOH to adjust its pH value to 7. The residue was purified by silica gel chromatography (0-10% MeOH in DCM with 1% NH₄OH_{(aq)}) to give P1.167 (300 mg, 91%) as a black powder. ¹H NMR (400 MHz, CD₃OD), δ: 7.70 (s, 1H), 7.39 (s, 1H), 7.32 (dd, J=8.4, 2.0 Hz, 1H), 7.21 (d, J=8.4 Hz, 1H), 3.68-3.64 (m, 1H), 3.62 (s, 3H), 3.03-3.00 (m, 1H), 2.88-2.79 (m, 1H), 2.39 (s, 3H), 2.34 (s, 3H); LCMS (ESI): 274.9 [M+H]⁺.

### Preparation 172. (S)-3-(6-Methyl-2-oxo-1,2-dihydroquinolin-3-yl)-2-(methylamino)propanamide (P1.168)

P1.167 (300 mg, 0.806 mmol) was dissolved in 7N NH₃ in MeOH (4 mL) in a sealed tube. The solution was heated at 80° C. for 3 days. After completion, the reaction mixture was concentrated under reduced pressure to give P1.168 (291 mg) as a black solid which was used in next step without further purification. ¹H NMR (400 MHz, DMSO-d₆), δ: 11.68 (br. s, 1H), 7.61 (s, 1H), 7.42-7.32 (m, 2H), 7.31-7.12 (m, 3H), 7.04 (s, 1H), 3.27 (t, J=7.0 Hz, 1H), 2.71-2.69 (m, J=6.8 Hz, 2H), 2.32 (s, 3H), 2.23 (s, 3H); LCMS (ESI): 260.3 [M+H]⁺.

### Synthesis of (1R,2S,5S)—N—((S)-1-amino-3-(6-methyl-2-oxo-1,2-dihydroquinolin-3-yl)-l-oxopropan-2-yl)-3-((S)-3,3-dimethyl-2-(methylsulfonamido)butanoyl)-N,6,6-trimethyl-3-azabicyclo[3.1.0]hexane-2-carboxamide (P1.169)

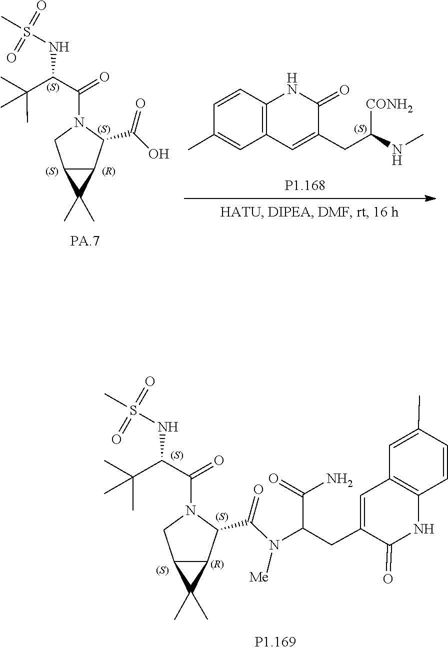

### Preparation 173. (1R,2S,5S)—N—((S)-1-Amino-3-(6-methyl-2-oxo-1,2-dihydroquinolin-3-yl)-l-oxopropan-2-yl)-3-((S)-3,3-dimethyl-2-(methylsulfonamido)butanoyl)-N,6,6-trimethyl-3-azabicyclo[3.1.0]hexane-2-carboxamide (P1.169)

To a solution of PA.7 (500 mg, 0.866 mmol, 60% purity) and P1.168 (150 mg, 0.577 mmol) in anh. DMF (6 mL) was added DIPEA (0.3 mL, 1.7 mmol) and HATU (241 mg, 0.635 mmol). After the mixture was stirred at 60° C. for 16 h, the mixture was diluted with EtOAc and washed with H₂O and brine. The organic layer was dried over MgSO₄, filtered, and concentrated under reduced pressure. The residue was purified by silica gel chromatography (0-5% MeOH in DCM with 1% NH₄OH, UV wavelength: 230 nm) to give P1.169 (75 mg, 9%) as a white solid. ¹H NMR (400 MHz, CD₃OD), δ: 7.89-7.77 (m, 1H), 7.50-7.27 (m, 2H), 7.27-7.14 (m, 1H), 5.69-5.67 (m, 1H), 4.64-4.53 (m, 1H), 3.86 (s, 1H), 3.79-3.63 (m, 1H), 3.46-3.34 (m, 1H), 3.22-2.75 (m, 7H), 2.47-2.28 (m, 4H), 1.26-1.17 (m, 6H), 1.05-0.71 (m, 11H); LCMS (ESI): 588.1 [M+H]⁺.

### Synthesis of tert-butyl ((2S)-1-((1R,2S,5S)-2-((1-amino-3-(6-methyl-2-oxo-1,2-dihydroquinolin-3-yl)-1-oxopropan-2-yl)carbamoyl)-6,6-dimethyl-3-azabicyclo[3.1.0]hexan-3-yl)-3,3-dimethyl-1-oxobutan-2-yl)carbamate (P1.170)

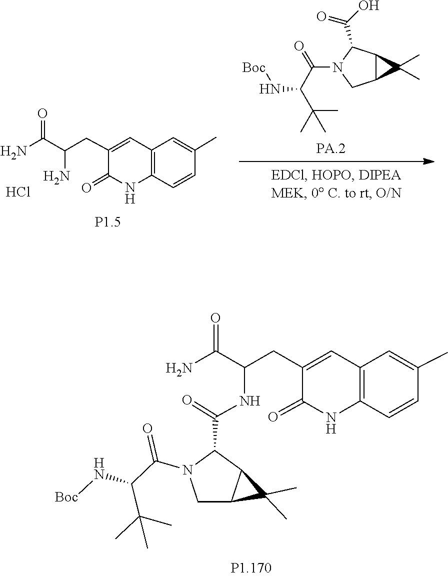 [SMILES: Cc1ccc2[nH]c(=O)c(CC(N)C(N)=O)cc2c1 ; CC(C)(C)OC(=O)N[C@H](C(=O)N1C[C@H]2[C@@H]([C@H]1C(=O)O)C2(C)C)C(C)(C)C ; Cl ; Cc1ccc2[nH]c(=O)c(CC(NC(=O)[C@@H]3[C@@H]4[C@H](CN3C(=O)[C@@H](NC(=O)OC(C)(C)C)C(C)(C)C)C4(C)C)C(N)=O)cc2c1]

### Preparation 174. tert-Butyl ((2S)-1-((1R,2S,5S)-2-((1-amino-3-(6-methyl-2-oxo-1,2-dihydroquinolin-3-yl)-1-oxopropan-2-yl)carbamoyl)-6,6-dimethyl-3-azabicyclo[3.1.0]hexan-3-yl)-3,3-dimethyl-1-oxobutan-2-yl)carbamate (P1.170)

To a solution of P1.5 (421 mg, 14.93 mmol, 1.1 eq), PA.2 (500 mg, 13.57 mmol, 1.0 eq) in butan-2-one (4.5 mL) was added 2-hydroxypyridine 1-oxide (38 mg, 3.39 mmol, 0.25 eq) and 1-(3-dimethylaminopropyl)-3-ethylcarbodiimide hydrochloride (338 mg, 17.64 mmol, 1.3 eq) at 0° C. DIPEA (0.945 mL, 54.28 mmol, 4.0 eq) was added slowly to the reaction mixture and the resulting mixture was stirred overnight. The solution was purified by C18 column (5-100% MeOH in H₂O containing 0.1% NH₄HCO₃) to give P1.170 (120 mg, 15%) as a white solid. ¹H NMR (400 MHz, CD₃OD), δ: 7.81-7.76 (m, 1H), 7.43-7.36 (m, 1H), 7.35-7.33 (m, 1H), 7.24-7.22 (m, 1H), 4.86-4.58 (m, 1H), 4.21-3.88 (m, 4H), 3.16-2.80 (m, 2H), 2.39-2.38 (m, 3H), 1.58-1.40 (m, 11H), 1.08-0.83 (m, 15H).

### Synthesis of 3-bromo-1-(4-methoxybenzyl)-1,6-naphthyridin-2(1H)-one (P1.174)

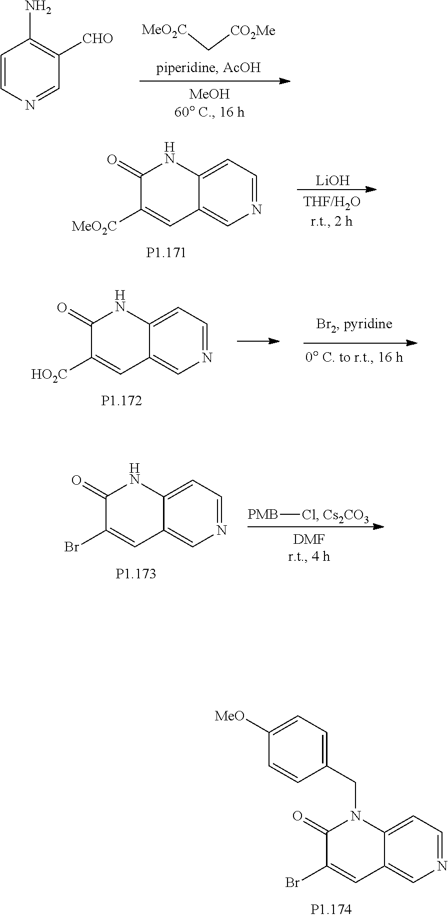

### Preparation 175. Methyl 2-oxo-1,2-dihydro-1,6-naphthyridine-3-carboxylate (P1.171)

To a solution of 4-aminonicotinaldehyde (500 mg, 4.09 mmol, 1.0 eq) and dimethyl malonate (1.25 mL, 11.0 mmol, 2.7 eq) in methanol was added piperidine (1.13 mL, 11.4 mmol, 2.8 eq) and acetic acid (23 μL, 0.409 mmol, 0.1 eq) at rt. After the reaction solution was stirred at 60° C. for 16 h, the reaction solution was concentrated under reduced pressure. The residue was treated with diethyl ether. The precipitate was collected by filtration and washed with diethyl ether to afford P1.171 (762 mg, 91%) as a pale-yellow solid. ¹H NMR (400 MHz, DMSO-d₆), δ: 8.95 (s, 1H), 8.59 (s, 1H), 8.50 (d, J=5.6 Hz, 1H), 7.19 (d, J=5.6 Hz, 1H), 3.80 (s, 3H).

### Preparation 176. 2-Oxo-1,2-dihydro-1,6-naphthyridine-3-carboxylic acid (P1.172)

To a solution of P1.171 (200 mg, 0.979 mmol, 1.0 eq) in THF (4.0 mL) and H₂O (4.0 mL) was added lithium hydroxide (70.3 mg, 2.93 mmol, 3.0 eq) at rt. After the reaction solution was stirred at rt for 2 h, the solution was concentrated under reduced pressure. The residue was acidified with 1N HCl_{(aq)} to pH 4. The precipitate was collected by filtration and washed with water to afford P1.172 (164 mg, 88%) as a white solid. ¹H NMR (400 MHz, DMSO-d₆), δ: 9.05 (s, 1H), 8.73 (s, 1H), 8.48 (d, J=5.6 Hz, 1H), 7.32 (d, J=5.6 Hz, 1H).

### Preparation 177. 3-Bromo-1,6-naphthyridin-2(1H)-one (P1.173)

To a solution of P1.172 (100 mg, 0.525 mmol, 1.0 eq) in pyridine (2.6 mL) was added bromine (54.0 μL, 1.05 mmol, 2.0 eq) dropwise at 0° C. After the reaction solution was stirred at 0° C. for 20 min, warmed up to rt and stirred for another 16 h. The reaction mixture was concentrated under reduced pressure to remove pyridine. The residue was purified by silica gel chromatography (6-14% of methanol in DCM) to afford P1.173 (48.9 mg, 41%) as an orange solid. ¹H NMR (400 MHz, CD₃OD), δ: 12.51 (br. s, 1H), 8.83 (s, 1H), 8.59 (s, 1H), 8.49 (d, J=5.6 Hz, 1H), 7.21 (d, J=5.6 Hz, 1H).

### Preparation 178. 3-Bromo-1-(4-methoxybenzyl)-1,6-naphthyridin-2(1H)-one (P1.174)

To a solution of P1.173 (48.9 mg, 0.217 mmol, 1.0 eq) in N,N-dimethylformamide (1.8 mL) was added 4-methoxybenzyl chloride (51.0 mg, 0.326 mmol, 1.5 eq) and cesium carbonate (141 mg, 0.434 mmol, 2.0 eq) at room temperature. After the reaction mixture was stirred at room temperature for 4 h, it was diluted with water and extracted with EtOAc. The organic layers were dried over MgSO₄₍ₛ₎, filtered, and concentrated under reduced pressure. The residue was purified by silica gel chromatography (50% EtOAc in dichloromethane) to afford P1.174 (50 mg, 66%) as a yellow solid. ¹H NMR (400 MHz, DMSO-d₆), δ: 8.89 (s, 1H), 8.68 (s, 1H), 8.53 (d, J=6.0 Hz, 1H), 7.45 (d, J=6.0 Hz, 1H), 7.18 (d, J=8.4 Hz, 2H), 6.86 (d, J=8.4 Hz, 2H), 5.45 (s, 2H), 3.68 (s, 3H).

### Synthesis of (R)-2-amino-3-(2-oxo-1,2-dihydro-1,6-naphthyridin-3-yl)propanamide (P1.177)

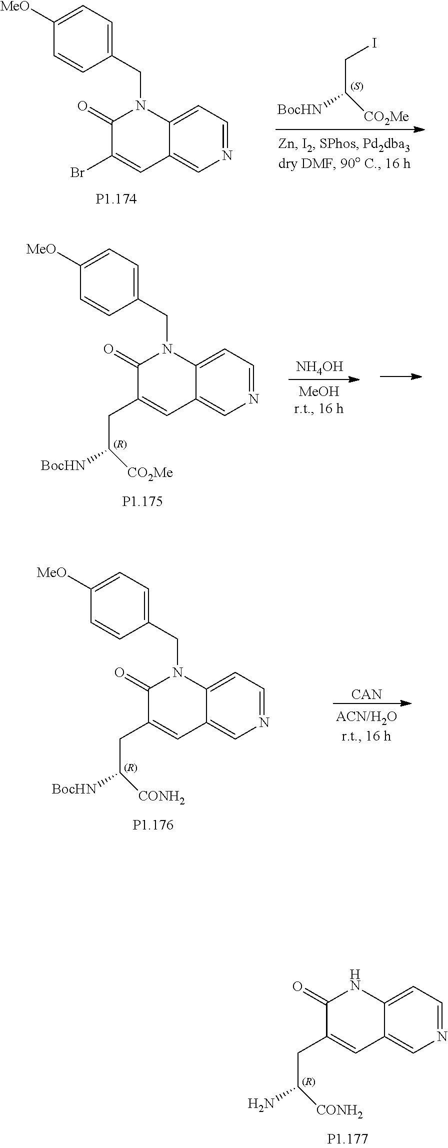

### Preparation 179. Methyl (R)-2-((tert-butoxycarbonyl)amino)-3-(1-(4-methoxybenzyl)-2-oxo-1,2-dihydro-1,6-naphthyridin-3-yl)propanoate (P1.175)

To a solution of zinc powder (1.13 g, 17.3 mmol, 6.0 eq) in anh. DMF (30 mL) was added iodine (0.220 g, 0.869 mmol, 0.3 eq) and stirred at rt. After the color of the solution was changed from red-brown to colorless, the solution was added methyl (S)-2-((tert-butoxycarbonyl)amino)-3-iodopropanoate (1.91 g, 5.79 mmol, 2.0 eq) and iodine (0.220 g, 0.869 mmol, 0.3 eq) and stirred at rt. After the color of the solution was changed from red-brown to colorless again, the solution was stirred for another 30 min and then was added P1.174 (1.00 g, 2.89 mmol, 1.0 eq) and 2-dicyclohexylphosphino-2′,6′-dimethoxybiphenyl (59.4 mg, 0.145 mmol, 0.05 eq) at rt. The mixture solution was degassed with Argon_{(g)} and then added by tris(dibenzylideneacetone)dipalladium(0) (66.3 mg, 0.0724 mmol, 0.025 eq). The solution was stirred at 90° C. for 16 h. The reaction solution was diluted with EtOAc and water, filtered, and washed with EtOAc. The filtrate was extracted with EtOAc and washed with water. The organic layers were dried over MgSO₄₍ₛ₎, filtered, and concentrated under reduced pressure. The residue was purified by silica gel chromatography (50% EtOAc in DCM) to afford P1.175 (1.01 g, 75%) as an orange solid. ¹H NMR (400 MHz, CDCl₃), δ: 8.73 (s, 1H), 8.44 (d, J=6.0 Hz, 1H), 7.66 (s, 1H), 7.20-7.11 (m, 3H), 6.83 (d, J=8.8 Hz, 2H), 5.56-5.39 (m, 3H), 4.72-4.66 (m, 1H), 3.76-3.74 (m, 6H), 3.31-3.27 (m, 1H), 3.01-2.96 (m, 1H), 1.32 (s, 9H).

### Preparation 180. tert-Butyl (R)-(1-amino-3-(1-(4-methoxybenzyl)-2-oxo-1,2-dihydro-1,6-naphthyridin-3-yl)-1-oxopropan-2-yl)carbamate (P1.176)

To a solution of P1.175 (1.01 g, 2.05 mmol, 1.0 eq) in methanol (20.5 mL) was added 30% ammonium hydroxide solution (10.2 mL) and stirred at rt for 16 h. The precipitate was collected and washed with methanol to afford P1.176 (683 mg, 70%) as a white solid. ¹H NMR (400 MHz, DMSO-d₆), δ: 8.78 (s, 1H), 8.41 (d, J=6.0 Hz, 1H), 7.82 (s, 1H), 7.31-7.22 (m, 4H), 7.08 (s, 1H), 6.88-6.81 (m, 3H), 5.57-5.27 (m, 2H), 4.35-4.29 (m, 1H), 3.68 (s, 3H), 3.15-3.10 (m, 1H), 2.65-2.59 (m, 1H), 1.13 (s, 9H).

### Preparation 181. (R)-2-amino-3-(2-oxo-1,2-dihydro-1,6-naphthyridin-3-yl)propanamide (P1.177)

To a solution of P1.176 (583 mg, 1.28 mmol, 1.0 eq) in acetonitrile (5.8 mL) and H₂O (0.58 mL) was added ceric ammonium nitrate (4.23 g, 7.73 mmol, 6.0 eq). After the solution was stirred at rt for 16 h, the reaction solution was concentrated under reduced pressure and diluted with methanol. The precipitate was collected and washed with methanol to afford P1.177 (259 mg, 84%) as a white solid. ¹H NMR (400 MHz, DMSO-d₆), δ: 12.97 (s, 1H), 9.22 (s, 1H), 8.65 (d, J=6.4 Hz, 1H), 8.16 (s, 3H), 8.01 (s, 1H), 7.89 (s, 1H), 7.59-7.56 (m, 2H), 4.03-4.01 (m, 1H), 3.08-2.91 (m, 2H).

### Synthesis of (1R,2S,5S)—N—((R)-1-amino-i-oxo-3-(2-oxo-1,2-dihydro-1,6-naphthyridin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.178)

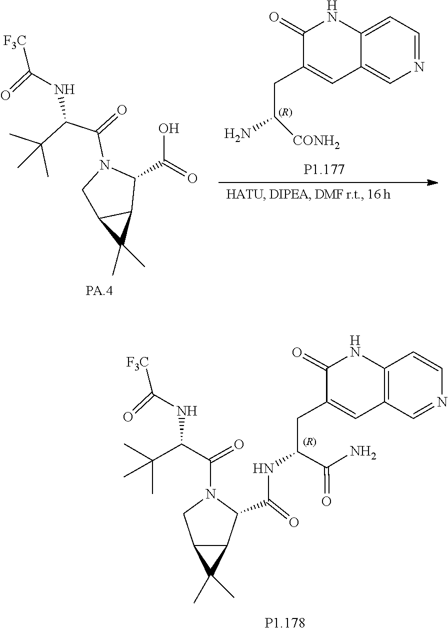

### Preparation 182. (1R,2S,5S)—N—((R)-1-Amino-i-oxo-3-(2-oxo-1,2-dihydro-1,6-naphthyridin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.178)

To a solution of PA.4 (52.3 mg, 0.143 mmol, 1.0 eq) in DMF (1 ml) was added P1.177 (40.0 mg, 0.172 mol, 1.2 eq), HATU (60.0 mg, 0.157 mmol, 1.1 eq) and DIPEA (75.0 μl, 0.430 mmol, 3.0 eq) at rt. After the reaction mixture was stirred at rt for 16 h, the reaction was purified by C18 flash column chromatography (5-100% methanol in H₂O) to afford P1.178 (56 mg, 67%) as a white solid. ¹H NMR (400 MHz, CD₃OD), δ: 8.77 (s, 1H), 8.42 (d, J=6.0 Hz, 1H), 7.89 (s, 1H), 7.26 (d, J=6.0 Hz, 1H), 4.84-4.81 (m, 1H), 4.52 (s, 1H), 4.22 (s, 1H), 3.93-3.76 (m, 2H), 3.39-3.34 (m, 1H), 2.87-2.81 (m, 1H), 2.21-2.17 (m, 1H), 1.39-1.36 (m, 1H), 1.07-0.87 (m, 15H).

### Synthesis of (1R,2S,5S)—N—((R)-1-amino-i-oxo-3-(2-oxo-1,2-dihydro-1,6-naphthyridin-3-yl)propan-2-yl)-3-((S)-3,3-dimethyl-2-(methylsulfonamido)butanoyl)-6,6-dimethyl-3-azabicyclo[3.1.0]hexane-2-carboxamide (P1.179)

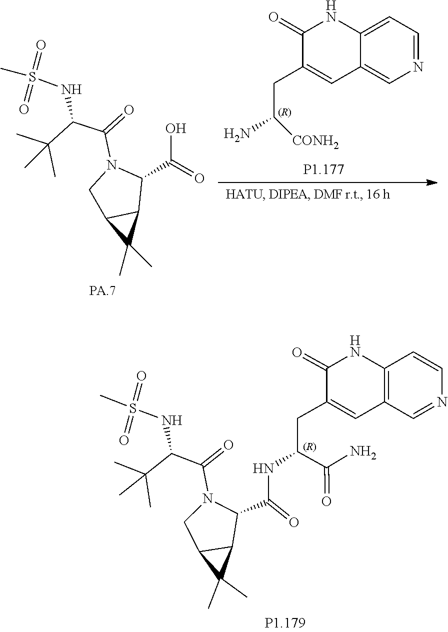

### Preparation 183. (1R,2S,5S)—N—((R)-1-amino-i-oxo-3-(2-oxo-1,2-dihydro-1,6-naphthyridin-3-yl)propan-2-yl)-3-((S)-3,3-dimethyl-2-(methylsulfonamido)butanoyl)-6,6-dimethyl-3-azabicyclo[3.1.0]hexane-2-carboxamide (P1.179)

To a solution of PA.7 (124 mg, 0.358 mmol, 1.0 eq) in DMF (2.3 ml) was added P1.177 (100 mg, 0.430 mol, 1.2 eq), HATU (150 mg, 0.394 mmol, 1.1 eq) and DIPEA (187 μl, 1.07 mmol, 3.0 eq) at rt. After the reaction mixture was stirred at rt for 16 h, purified by C18 flash column chromatography (5-100% methanol in H₂O) to afford P1.179 (93 mg, 46%) as a white solid. ¹H NMR (400 MHz, CD₃OD), δ: 8.77 (s, 1H), 8.42 (d, J=6.0 Hz, 1H), 7.89 (s, 1H), 7.26 (d, J=6.0 Hz, 1H), 4.85-4.81 (m, 1H), 4.23 (s, 1H), 3.89-3.85 (m, 2H), 3.77-3.75 (m, 1H), 3.42-3.37 (m, 1H), 2.92-2.80 (m, 4H), 1.43-1.40 (m, 1H), 1.10-1.00 (m, 9H), 0.98-0.88 (m, 7H).

### Synthesis of (S)-2-amino-3-(2-oxo-1,2-dihydro-1,6-naphthyridin-3-yl)propanamide (P1.182)

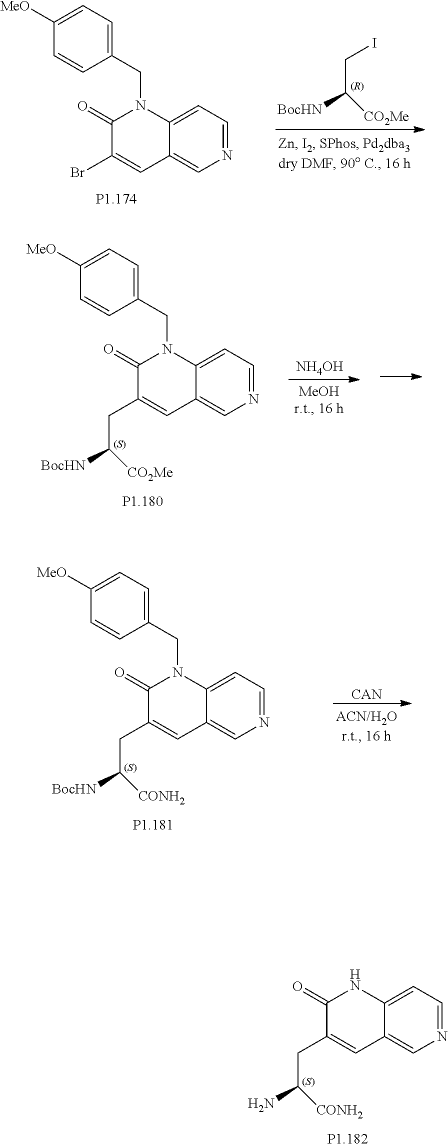

### Preparation 184. Methyl (S)-2-((tert-butoxycarbonyl)amino)-3-(1-(4-methoxybenzyl)-2-oxo-1,2-dihydro-1,6-naphthyridin-3-yl)propanoate (P1.180)

To a solution of Zinc (1.13 g, 17.3 mmol, 6.0 eq) in anh. DMF (30 mL) was added iodine (0.220 g, 0.869 mmol, 0.3 eq) and stirred at rt. After the color of the solution was changed from red-brown to colorless, the solution was added methyl (R)-2-((tert-butoxycarbonyl)amino)-3-iodopropanoate (1.91 g, 5.79 mmol, 2.0 eq) and iodine (0.220 g, 0.869 mmol, 0.3 eq) and stirred at rt. The color of the solution was changed from red-brown to colorless again. The solution was stirred for another 30 min and then was added P1.174 (1.00 g, 2.89 mmol, 1.0 eq) and 2-dicyclohexylphosphino-2′,6′-dimethoxybiphenyl (59.4 mg, 0.145 mmol, 0.05 eq) at rt. After the mixture solution was degassed with Argon_{(g)} and added by tris(dibenzylideneacetone)dipalladium(0) (66.3 mg, 0.0724 mmol, 0.025 eq), the reaction was stirred at 90° C. for 16 h. The reaction solution was diluted with EtOAc/water, filtered, and washed with EtOAc. The filtrate was extracted with EtOAc and washed with water. The organic layers were dried over MgSO₄₍ₛ₎, filtered and concentrated under reduced pressure. The residue was purified by silica gel chromatography (50% EtOAc in DCM) to afford P1.180 (1.06 g, 78%) as a yellow solid. ¹H NMR (400 MHz, CDCl₃), δ: 8.73 (s, 1H), 8.44 (d, J=6.0 Hz, 1H), 7.66 (s, 1H), 7.20-7.12 (m, 3H), 6.84 (d, J=8.8 Hz, 2H), 5.54-5.43 (m, 3H), 4.70-4.67 (m, 1H), 3.76-3.74 (m, 6H), 3.32-3.28 (m, 1H), 3.01-2.98 (m, 1H), 1.32 (s, 9H).

### Preparation 185. tert-Butyl (S)-(1-amino-3-(1-(4-methoxybenzyl)-2-oxo-1,2-dihydro-1,6-naphthyridin-3-yl)-1-oxopropan-2-yl)carbamate (P1.181)

To a solution of P1.180 (958 mg, 2.05 mmol, 1.0 eq) in methanol (20.5 mL) was added 30% ammonium hydroxide solution (10.2 mL) and stirred at rt for 16 h. The precipitate was collected and washed with methanol to afford P1.181 (417 mg, 45%) as a white solid. ¹H NMR (400 MHz, DMSO-d₆), δ: 8.78 (s, 1H), 8.41 (d, J=6.0 Hz, 1H), 7.82 (s, 1H), 7.31-7.22 (m, 4H), 7.08 (s, 1H), 6.88-6.81 (m, 3H), 5.57-5.27 (m, 2H), 4.35-4.29 (m, 1H), 3.68 (s, 3H), 3.15-3.10 (m, 1H), 2.65-2.59 (m, 1H), 1.13 (s, 9H).

### Preparation 186. (S)-2-Amino-3-(2-oxo-1,2-dihydro-1,6-naphthyridin-3-yl)propanamide (P1.182)

To a solution of P1.181 (203 mg, 0.448 mmol, 1.0 eq) in acetonitrile (2.0 mL) and H₂O (0.2 mL) was added ceric ammonium nitrate (1.47 g, 2.69 mmol, 6.0 eq) and stirred at rt for 16 h. The reaction solution was concentrated under reduced pressure and diluted with methanol. The precipitate was collected and washed with methanol to afford P1.182 (138 mg, 100%) as a yellow solid. ¹H NMR (400 MHz, DMSO-d₆), δ: 12.89 (br. s, 1H), 9.17 (s, 1H), 8.63 (d, J=6.4 Hz, 1H), 8.15 (br. s, 3H), 7.99 (s, 1H), 7.89 (s, 1H), 7.59 (s, 1H), 7.53 (d, J=6.4 Hz, 1H), 4.04-4.00 (m, 1H), 3.07-3.02 (m, 1H), 2.95-2.90 (m, 1H).

### Synthesis of (1R,2S,5S)—N—((S)-1-amino-i-oxo-3-(2-oxo-1,2-dihydro-1,6-naphthyridin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.183)

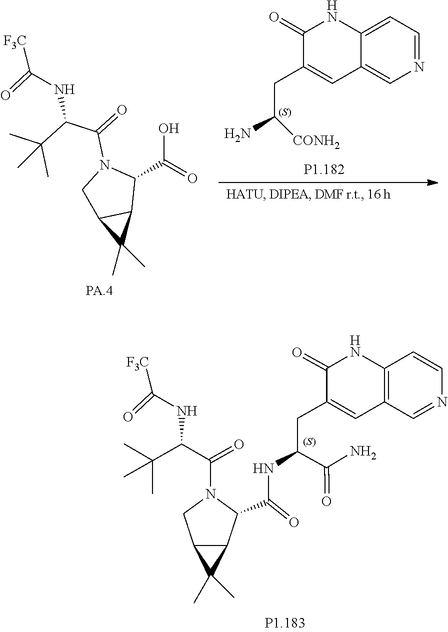

### Preparation 187. (1R,2S,5S)—N—((S)-1-amino-i-oxo-3-(2-oxo-1,2-dihydro-1,6-naphthyridin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.183)

To a solution of PA.4 (65.3 mg, 0.179 mmol, 1.0 eq) in DMF (1.2 mL) was added P1.182 (50.0 mg, 0.215 mol, 1.2 eq), HATU (75.0 mg, 0.197 mmol, 1.1 eq) and DIPEA (93.7 μl, 0.538 mmol, 3.0 eq) at rt. After the reaction mixture was stirred at rt for 16 h, the solution was purified by C18 flash column chromatography (5-100% methanol in H₂O containing 0.1% NH₄HCO₃) to afford P1.183 (77 mg, 74%) as a white solid. ¹H NMR (400 MHz, CD₃OD), δ: 8.82 (s, 1H), 8.41 (d, J=6.0 Hz, 1H), 7.96 (s, 1H), 7.25 (d, J=6.0 Hz, 1H), 4.70-4.66 (m, 1H), 4.53 (s, 1H), 4.25 (s, 1H), 4.05-4.01 (m, 1H), 3.79-3.77 (m, 1H), 3.19-3.14 (m, 1H), 3.02-2.96 (m, 1H), 1.56-1.52 (m, 1H), 1.35-1.33 (m, 1H), 1.07-0.79 (m, 15H).

### Synthesis of (1R,2S,5S)—N—((S)-1-amino-i-oxo-3-(2-oxo-1,2-dihydro-1,6-naphthyridin-3-yl)propan-2-yl)-3-((S)-3,3-dimethyl-2-(methylsulfonamido)butanoyl)-6,6-dimethyl-3-azabicyclo[3.1.0]hexane-2-carboxamide (P1.184)

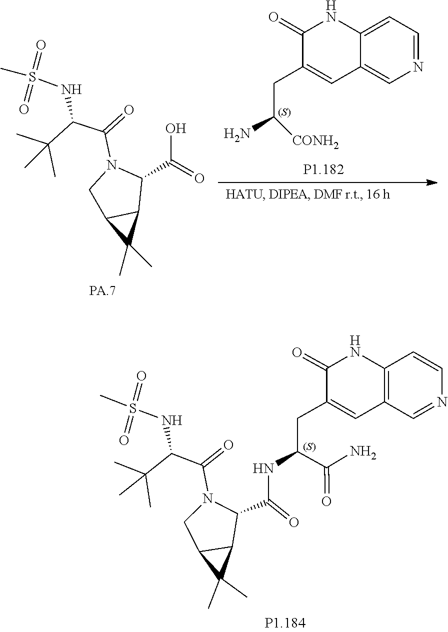

### Preparation 188. (1R,2S,5S)—N—((S)-1-amino-i-oxo-3-(2-oxo-1,2-dihydro-1,6-naphthyridin-3-yl)propan-2-yl)-3-((S)-3,3-dimethyl-2-(methylsulfonamido)butanoyl)-6,6-dimethyl-3-azabicyclo[3.1.0]hexane-2-carboxamide (P1.184)

To a solution of PA.7 (82.8 mg, 0.143 mmol, 1.0 eq) in DMF (1.0 mL) was added P1.182 (40.0 mg, 0.172 mol, 1.2 eq), HATU (60.0 mg, 0.157 mmol, 1.1 eq) and DIPEA (75 μl, 0.430 mmol, 3.0 eq) at rt. After the reaction mixture was stirred at rt for 16 h, the solution was purified by C18 flash column chromatography (5-100% methanol in H₂O containing 0.1% TFA) to afford P1.184 (TFA salt, 56 mg, 58%) as a white solid. ¹H NMR (400 MHz, DMSO-d₆), δ: 12.92 (s, 1H), 9.23 (s, 1H), 8.61 (d, J=6.4 Hz, 1H), 8.42 (d, J=8.4 Hz, 1H), 7.99 (s, 1H), 7.54 (d, J=6.4 Hz, 1H), 7.32 (s, 1H), 7.12 (s, 1H), 6.61 (d, J=8.4 Hz, 1H), 4.49-4.43 (m, 1H), 4.21 (s, 1H), 3.78-3.75 (m, 1H), 3.65-3.62 (m, 1H), 3.15 (s, 1H), 3.05-3.00 (m, 1H), 2.84 (s, 3H), 2.82-2.74 (m, 1H), 1.49-1.46 (m, 1H), 1.30-1.28 (m, 1H), 1.00-0.97 (m, 4H), 0.86-0.84 (m, 10H), 0.73-0.71 (m, 1H).

### Synthesis of (1R,2S,5S)—N—((S)-1-amino-3-(6-methyl-2-oxo-1,2-dihydroquinolin-3-yl)-1-oxopropan-2-yl)-6,6-dimethyl-3-(2-(3-methyl-¹H-pyrazol-5-yl)acetyl)-3-azabicyclo[3.1.0]hexane-2-carboxamide (P1.189)

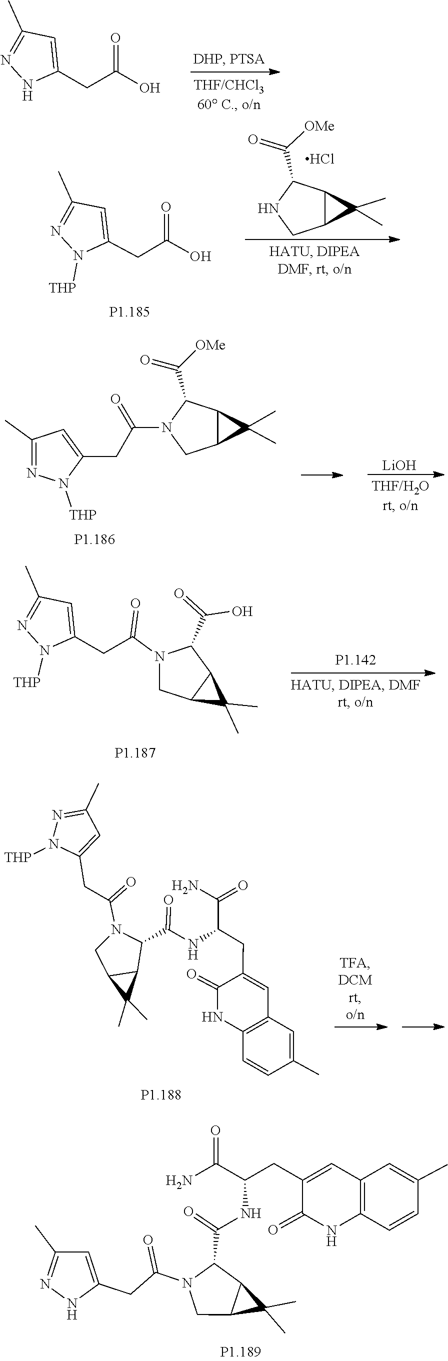

### Preparation 189. 2-(3-Methyl-1-(tetrahydro-2H-pyran-2-yl)-1H-pyrazol-5-yl)acetic acid (P1.185)

To a solution of 2-(3-methyl-1H-pyrazol-5-yl)acetic acid (300 mg, 2.14 mmol) and 3,4-dihydro-2H-pyran (0.8 mL, 8.57 mmol) in THF (3 mL) and chloroform (6 mL) was added 4-methylbenzenesulfonic acid (122 mg, 0.64 mmol) at rt. After the solution was stirred at 60° C. for overnight, the mixture was concentrated. The residue was purified by C18 reverse phase column (5-100% methanol in H₂O) to give P1.185 (370 mg, 77% yield) as a yellow oil. ¹H NMR (400 MHz, MeOD-d₄), δ: 6.05-6.04 (m, 1H), 5.33-5.30 (m, 1H), 4.02-3.68 (m, 2H), 3.56 (s, 2H), 2.32 (s, 3H), 2.07-2.03 (m, 2H), 1.99-1.56 (m, 4H).

### Preparation 190. Methyl (1R,2S,5S)-6,6-dimethyl-3-(2-(3-methyl-1-(tetrahydro-2H-pyran-2-yl)-1H-pyrazol-5-yl)acetyl)-3-azabicyclo[3.1.0]hexane-2-carboxylate (P1.186)

To a solution of P1.185 (370 mg, 1.65 mmol), and methyl (1R,2S,5S)-6,6-dimethyl-3-azabicyclo[3.1.0]hexane-2-carboxylate hydrochloride (356 mg, 1.73 mmol) in DMF (16 mL) was added HATU (752 mg, 1.98 mmol) and DIPEA (0.826 mL, 4.95 mmol) at 0° C. After the mixture was stirred at room temperature for overnight, the mixture was concentrated. The residue was purified by silica gel column chromatography (0-50% EtOAc in n-hexane) to give P1.186 (426 mg, 69%) as a yellow solid. ¹H NMR (400 MHz, MeOD-d₄), δ: 6.02-5.95 (m, 1H), 5.33-5.29 (m, 1H), 4.64-4.28 (m, 1H), 3.98-3.97 (m, 1H), 3.85-3.51 (m, 8H), 2.32-2.29 (m, 3H), 2.04-1.43 (m, 8H), 1.08-0.77 (m, 6H); LCMS (ESI) m/z 376.1 [M+H]⁺.

### Preparation 191. (1R,2S,5S)-6,6-Dimethyl-3-(2-(3-methyl-1-(tetrahydro-2H-pyran-2-yl)-1H-pyrazol-5-yl)acetyl)-3-azabicyclo[3.1.0]hexane-2-carboxylic acid (P1.187)

To a solution of P1.186 (170 mg, 0.45 mmol) in THF (2.5 mL) was added water (2.5 mL) and lithium hydroxide (33 mg, 1.38 mmol) at rt. After the mixture was stirred for overnight, the mixture was acidified by slow addition of 1 N HCl_{(aq)}, adjusted pH to 6, and extracted with DCM three times. The combined organic layers were dried over Na₂SO₄₍ₛ₎, filtered, and concentrated in vacuo to give crude P1.187 (125 mg, 77%) as a white solid. ¹H NMR (400 MHz, MeOD-d₄), δ: 6.01-5.96 (m, 1H), 5.32-5.29 (m, 1H), 4.55-4.26 (m, 1H), 4.01-3.98 (m, 1H), 3.86-3.81 (m, 1H), 3.74-3.50 (m, 4H), 2.30-2.29 (m, 3H), 2.05-1.48 (m, 8H), 1.05-0.78 (m, 6H); LCMS (ESI) m/z 360.0 [M−H]⁻.

### Preparation 192. (1R,2S,5S)—N—((S)-1-Amino-3-(6-methyl-2-oxo-1,2-dihydroquinolin-3-yl)-1-oxopropan-2-yl)-6,6-dimethyl-3-(2-(3-methyl-1-(tetrahydro-2H-pyran-2-yl)-1H-pyrazol-5-yl)acetyl)-3-azabicyclo[3.1.0]hexane-2-carboxamide (P1.188)

To a solution of P1.187 (100 mg, 0.28 mmol) in DMF (3 mL) was added P1.142 (204 mg, 0.83 mmol), HATU (158 mg, 0.42 mmol) and DIPEA (0.145 mL, 0.83 mmol). The reaction mixture was stirred at rt for overnight. The mixture was treated with water and extracted with EtOAc three times. The combined organic layers were washed with brine, dried over Na₂SO₄₍ₛ₎, filtered, and concentrated in vacuo. The residue was purified by silica gel column chromatography (0-7% methanol in DCM) to give P1.188 (75 mg, 46%) as a yellow solid. ¹H NMR (400 MHz, MeOD-d₄), δ: 7.81-7.76 (m, 1H), 7.43-7.22 (m, 3H), 5.83-5.68 (m, 1H), 5.29-5.25 (m, 1H), 4.60-4.56 (m, 1H), 4.24-4.18 (m, 1H), 4.13-4.07 (m, 1H), 4.00-4.98 (m, 1H), 3.83-3.64 (m, 4H), 3.24-3.00 (m, 2H), 2.39 (s, 3H), 2.29-2.19 (m, 3H), 1.84-1.51 (m, 5H), 1.38-1.22 (m, 2H), 0.97-0.70 (m, 6H); LCMS (ESI) m/z 589.2 [M+H]⁺.

### Preparation 193. (1R,2S,5S)—N—((S)-1-Amino-3-(6-methyl-2-oxo-1,2-dihydroquinolin-3-yl)-1-oxopropan-2-yl)-6,6-dimethyl-3-(2-(3-methyl-1H-pyrazol-5-yl)acetyl)-3-azabicyclo[3.1.0]hexane-2-carboxamide (P1.189)

A solution of P1.188 (60 mg, 0.1 mmol) in DCM (0.5 mL) and TFA (0.125 mL) was stirred at rt overnight. The mixture was treated with NaHCO_{3(aq)} and extracted with DCM. The combined organic layers were washed with brine, dried over Na₂SO₄₍ₛ₎, filtered, and concentrated in vacuo to give crude P1.189 (50 mg, 98%) as a yellow solid. ¹H NMR (400 MHz, MeOD-d₄), δ: 7.79-7.77 (m, 1H), 7.42-7.22 (m, 3H), 5.82-5.49 (m, 1H), 4.61-4.12 (m, 2H), 3.74-3.54 (m, 4H), 3.23-3.02 (m, 2H), 2.39 (s, 3H), 2.17 (s, 3H), 1.45-1.27 (m, 2H), 0.95-0.82 (m, 6H); LCMS (ESI) m/z 505.1 [M+H]⁺.

### Synthesis of (1R,2S,5S)—N—((S)-1-amino-3-(6-methyl-2-oxo-1,2-dihydroquinolin-3-yl)-1-oxopropan-2-yl)-6,6-dimethyl-3-(2-(3-(trifluoromethyl)-1H-pyrazol-5-yl)acetyl)-3-azabicyclo[3.1.0]hexane-2-carboxamide (P1.197)

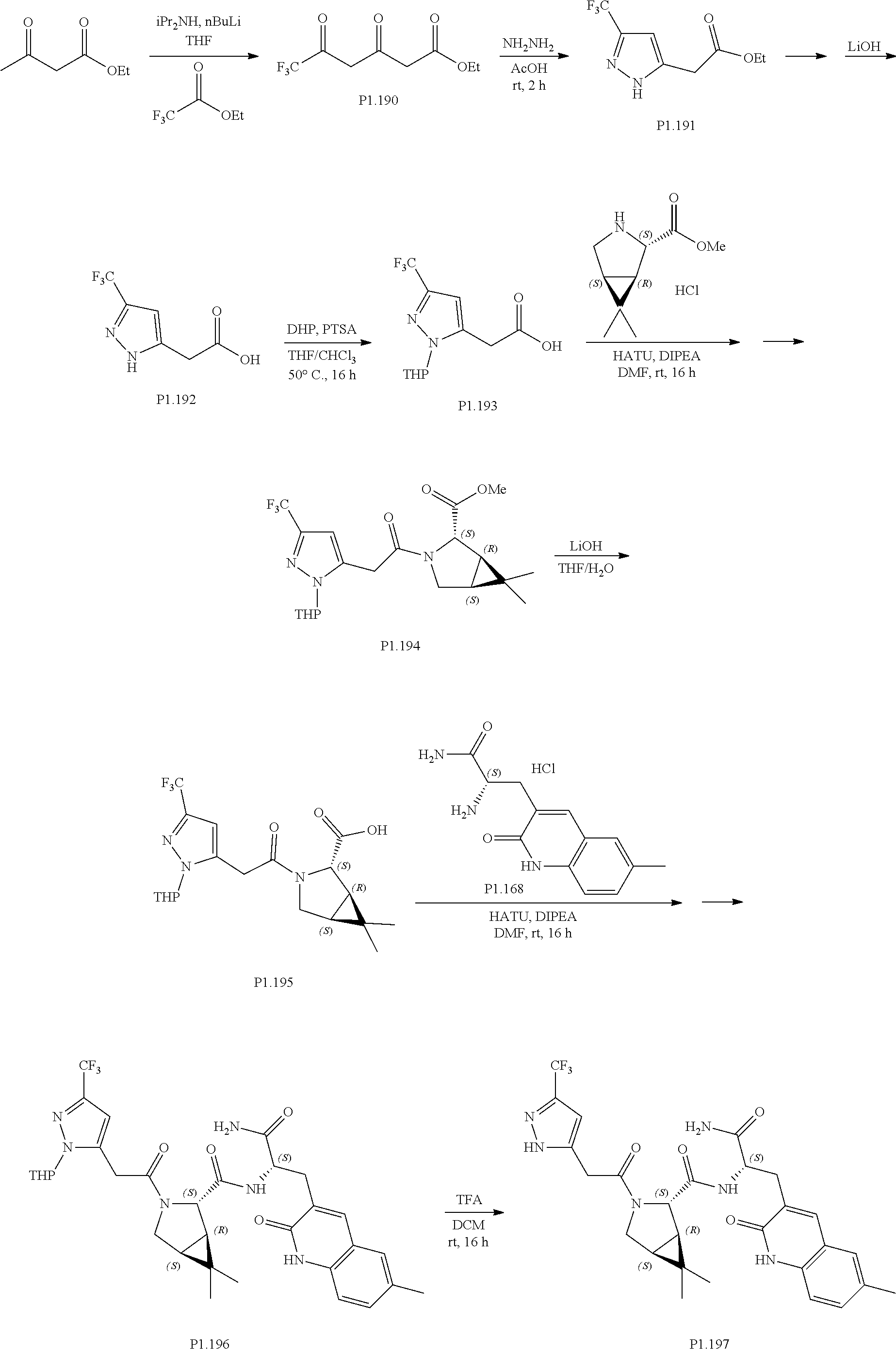

### Preparation 194. Ethyl 6,6,6-trifluoro-3,5-dioxohexanoate (P1.190)

To a solution of 1.6 M n-butyllithium in hexanes (71.3 mL, 114 mmol, 2.97 eq) in anh. THF (96 mL) was added N,N-diisopropylamine (16.1 mL, 115 mmol, 3.0 eq) at 0° C. After the reaction mixture was stirred at 0° C. for 30 min, ethyl 3-oxobutanoate was added to the prepared solution and it was stirred at 0° C. for 1 h. After the reaction solution was cooled to −78° C., ethyl 2,2,2-trifluoroacetate was added dropwise to the solution and the reaction was slowly warmed to rt. The reaction solution was stirred at rt for 16 h, cooled to 0° C., and acidified with 2N HCl_{(aq)} to pH 4. The mixture solution was extracted with DCM. The organic layers were washed with water, collected, washed with brine, dried over MgSO₄₍ₛ₎, filtered, and concentrated in vacuo to afford P1.190 (9.15 g) as an orange liquid. ¹H NMR (400 MHz, CDCl₃), δ: 6.09 (s, 1H), 4.23 (q, J=7.2 Hz, 2H), 3.48 (s, 2H), 1.30 (t, J=7.2 Hz, 3H).

### Preparation 195. Ethyl 2-(3-(trifluoromethyl)-1H-pyrazol-5-yl)acetate (P1.191)

To a solution of P1.190 (5.95 g, 26.3 mmol, 1.0 eq) in acetic acid (66 mL) was added hydrazine monohydrate (1.53 mL, 31.5 mmol, 1.2 eq) at 0° C. After the reaction solution was stirred at rt for 2 h, the solution was cooled to 0° C. and quenched with NaOH_{(aq)} to pH 8. The mixture solution was extracted with EtOAc. The organic layers were washed with water, dried over MgSO₄₍ₛ₎, filtered, and concentrated in vacuo. The residue was purified by silica gel chromatography (EtOAc:n-hexane=1:1) to afford P1.191 (1.90 g, 32%) as a yellow oil. ¹H NMR (400 MHz, CDCl₃), δ: 6.43 (s, 1H), 6.03 (s, 1H), 4.24 (q, J=7.2 Hz, 2H), 3.79 (s, 2H), 1.31 (t, J=7.2 Hz, 3H).

### Preparation 196. 2-(3-(Trifluoromethyl)-1H-pyrazol-5-yl)acetic acid (P1.192)

To a stirred solution of P1.191 (1.96 g, 8.82 mmol, 1.0 eq) in THF (15 mL) and H₂O (15 mL) was added lithium hydroxide (431 mg, 18.0 mmol, 3.0 eq) at rt. After the solution was stirred at rt for 16 h, it was concentrated in vacuo and diluted with water. The solution was acidified to pH 4 with HCl_{(aq)}. The mixture solution was extracted with EtOAc. The organic layers were collected, washed with brine, dried over MgSO₄₍ₛ₎, filtered, and concentrated in vacuo to afford P1.192 (1.58 g, 92%) as a yellow solid. ¹H NMR (400 MHz, DMSO-d₆), δ: 13.44 (br. s, 1H), 12.69 (br. s, 1H), 6.54 (s, 1H), 3.72 (s, 2H).

### Preparation 197. 2-(1-(Tetrahydro-2H-pyran-2-yl)-3-(trifluoromethyl)-1H-pyrazol-5-yl)acetic acid (P1.193)

To a solution of P1.192 (1.58 g, 8.13 mmol, 1.0 eq) in THF (20 mL) and chloroform (40 mL) was added p-toluenesulfonic acid (232 mg, 1.22 mmol, 0.15 eq) and 3,4-dihydropyran (1.48 mL, 16.2 mmol, 2.0 eq) at rt. After the solution was stirred at 50° C. for 16 h, it was concentrated in vacuo and purified by C18 flash column (5-100% methanol in H₂O) to afford P1.193 (1.30 g, 57%) as a yellow solid. ¹H NMR (400 MHz, DMSO-d₆), δ: 12.74 (br. s, 1H), 6.67 (s, 1H), 5.50 (dd, J=9.6, 2.8 Hz, 1H), 3.90-3.80 (m, 3H), 3.72-3.60 (m, 1H), 2.22-2.12 (m, 1H), 1.98-1.86 (m, 2H), 1.69-1.49 (m, 3H).

### Preparation 198. Methyl (1R,2S,5S)-6,6-dimethyl-3-(2-(1-(tetrahydro-2H-pyran-2-yl)-3-(trifluoromethyl)-1H-pyrazol-5-yl)acetyl)-3-azabicyclo[3.1.0]hexane-2-carboxylate (P1.194)

To a solution of P1.193 (200 mg, 0.718 mmol, 1.0 eq) in DMF (7 mL) was added methyl (1R,2S,5S)-6,6-dimethyl-3-azabicyclo[3.1.0]hexane-2-carboxylate (177 mg, 0.862 mol, 1.2 eq), HATU (300 mg, 0.791 mmol, 1.1 eq) and DIPEA (375 μl, 2.15 mmol, 3.0 eq) at rt. After the reaction solution was stirred at rt for 16 h, the solution was diluted with water and extracted with EtOAc. The organic layers were collected, washed with brine, dried over MgSO₄₍ₛ₎, filtered, and concentrated in vacuo. The residue was purified by C18 flash column (5-100% methanol in H₂O) to afford P1.194 (265 mg, 86%) as a yellow oil. ¹H NMR (400 MHz, CDCl₃), δ: 6.50-6.39 (m, 1H), 5.43-5.40 (m, 1H), 4.42-4.28 (m, 1H), 3.97-3.51 (m, 9H), 2.40-2.31 (m, 1H), 2.12-2.02 (m, 2H), 1.72-1.63 (m, 2H), 1.57-1.45 (m, 3H), 1.06 (s, 3H), 0.96-0.88 (m, 3H).

### Preparation 199. (1R,2S,5S)-6,6-Dimethyl-3-(2-(1-(tetrahydro-2H-pyran-2-yl)-3-(trifluoromethyl)-1H-pyrazol-5-yl)acetyl)-3-azabicyclo[3.1.0]hexane-2-carboxylic acid (P1.195)

To a solution of P1.194 (265 mg, 0.617 mmol, 1.0 eq) in H₂O (2 mL) and THF (2 mL) was added lithium hydroxide (44.3 mg, 1.85 mmol, 3.0 eq) at rt. After the solution was stirred at rt for 16 h, it was concentrated in vacuo and diluted with water. The solution was acidified to pH 4 with HCl_{(aq)}. The mixture solution was extracted with EtOAc. The organic layers were collected, washed with brine, dried over MgSO₄₍ₛ₎, filtered, and concentrated in vacuo to afford P1.195 (260 mg, crude) as a yellow oil. ¹H NMR (400 MHz, CDCl₃), δ: 12.77 (br. s, 1H), 6.65-6.55 (m, 1H), 5.39-5.34 (m, 1H), 4.55-4.10 (m, 1H), 3.95-3.45 (m, 6H), 2.18-2.10 (m, 1H), 1.93-1.81 (m, 2H), 1.62-1.41 (m, 5H), 1.02-0.88 (m, 6H).

### Preparation 200. (1R,2S,5S)—N—((S)-1-Amino-3-(6-methyl-2-oxo-1,2-dihydroquinolin-3-yl)-1-oxopropan-2-yl)-6,6-dimethyl-3-(2-(1-(tetrahydro-2H-pyran-2-yl)-3-(trifluoromethyl)-1H-pyrazol-5-yl)acetyl)-3-azabicyclo[3.1.0]hexane-2-carboxamide (P1.196)

To a solution of P1.195 (150 mg, 0.361 mmol) and P1.168 (120 mg, 0.433 mmol) in DMF (3.6 mL) was added DIPEA (0.19 mL, 1.1 mmol) and HATU (151 mg, 0.397 mmol). After the mixture was stirred at rt for 16 h, the mixture was diluted with EtOAc and washed with H₂O and brine. The organic layer was dried over MgSO₄, filtered, and concentrated under reduced pressure. The residue was purified by silica gel chromatography (0-5% MeOH in CH₂Cl₂ containing 1% NH₄OH_{(aq)}) to give P1.196 (174 mg, 75%) as a yellow powder. ¹H NMR (400 MHz, CD₃OD), δ: 7.87-7.71 (m, 1H), 7.47-6.95 (m, 3H), 6.48-6.06 (m, 1H), 5.53-5.07 (m, 1H), 4.62-4.46 (m, 1H), 4.29-3.36 (m, 8H), 3.25-2.85 (m, 3H), 2.42-2.27 (m, 4H), 2.07-1.80 (m, 3H), 1.72-1.27 (m, 6H), 1.06-0.90 (m, 6H); LCMS (ESI) m/z calc. for C₃₂H₃₇F₃N₆O₅ 642.28. found, 643.1 [M+H]⁺.

### Preparation 201. (1R,2S,5S)—N—((S)-1-Amino-3-(6-methyl-2-oxo-1,2-dihydroquinolin-3-yl)-1-oxopropan-2-yl)-6,6-dimethyl-3-(2-(3-(trifluoromethyl)-1H-pyrazol-5-yl)acetyl)-3-azabicyclo[3.1.0]hexane-2-carboxamide (P1.197)

To a solution of P1.196 (160 mg, 0.249 mmol) in CH₂Cl₂ (5 mL) was added TFA (2.0 mL). After the mixture was stirred at rt for 16 h, the mixture was diluted with DCM and washed with NaHCO_{3(aq)} and brine. The organic layer was dried over MgSO₄, filtered, and concentrated under reduced pressure to give P1.197 (141 mg, 99%) as a yellow powder which was used in the next step without purification. ¹H NMR (400 MHz, CD₃OD), δ: 7.82-7.76 (m, 1H), 7.45-7.28 (m, 2H), 7.27-7.06 (m, 1H), 6.44-6.12 (m, 1H), 4.59-4.52 (m, 1H), 4.27-4.14 (m, 2H), 4.02-3.87 (m, 2H), 3.71-3.57 (m, 1H), 3.18-2.96 (m, 2H), 2.41-2.30 (m, 3H), 1.59-1.55 (m, 1H), 1.39-1.35 (m, 1H), 1.01-0.89 (m, 6H); LCMS (ESI) m/z calc. for C₂₇H₂₉F₃N₆O₄ 558.22. found, 559.1 [M+H]⁺.

### Synthesis of (1R,2S,5S)—N—((S)-1-amino-3-(6-methyl-2-oxo-1,2-dihydroquinolin-3-yl)-1-oxopropan-2-yl)-6,6-dimethyl-3-(2-(3-methylisoxazol-5-yl)acetyl)-3-azabicyclo[3.1.0]hexane-2-carboxamide (P1.200)

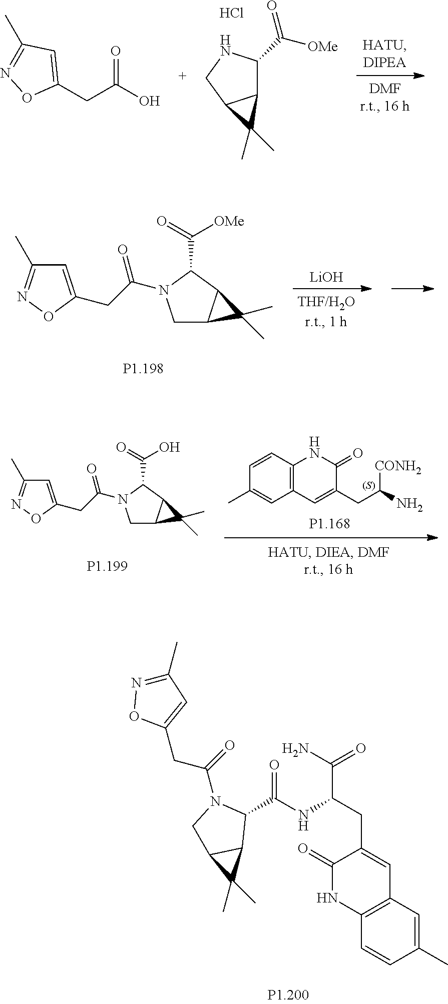

### Preparation 202. Methyl (1R,2S,5S)-6,6-dimethyl-3-(2-(3-methylisoxazol-5-yl)acetyl)-3-azabicyclo[3.1.0]hexane-2-carboxylate (P1.198)

To a solution of 2-(3-methylisoxazol-5-yl)acetic acid (100.0 mg, 0.709 mmol, 1.0 eq), methyl (1R,2S,5S)-6,6-dimethyl-3-azabicyclo[3.1.0]hexane-2-carboxylate hydrochloride (148.7 mg, 0.723 mmol, 1.02 eq) in DMF (1.0 mL) was added DIPEA (0.37 mL, 2.13 mmol, 3.0 eq) and HATU (323.3 mg, 0.850 mmol, 1.2 eq) at 0° C. The reaction mixture was stirred at 0° C. for 30 min and then stirred at rt for 16 h. The solution was treated with water and extracted with DCM three times. The combined organic layers were washed with brine, dried over Na₂SO₄₍ₛ₎, filtered, and concentrated in vacuo to give the residue. The residue was purified by silica gel column chromatography (n-hexane:EtOAc=2:1 to 1:1) to give P1.198 (198 mg, 96% yield) as a colorless oil. ¹H NMR (400 MHz, CDCl₃), δ: 6.16-6.09 (m, 1H), 4.41-4.37 (m, 1H), 3.87 (dd, J=10.0, 5.2 Hz, 1H), 3.80-3.72 (m, 5H), 3.59-3.54 (m, 1H), 2.27-2.26 (m, 3H), 1.53-1.41 (m, 2H), 1.06 (s, 3H), 0.94-0.90 (m, 3H).

### Preparation 203. (1R,2S,5S)-6,6-Dimethyl-3-(2-(3-methylisoxazol-5-yl)acetyl)-3-azabicyclo [3.1.0]hexane-2-carboxylic acid (P1.199)

To a solution of P1.198 (168.0 mg, 0.575 mmol, 1.0 eq) in THF (2.6 mL) and water (2.6 mL) was added lithium hydroxide (41.3 mg, 1.72 mmol, 3.0 eq). The reaction was stirred at rt for 1 h. The mixture was acidified by slow addition of 1 N HCl_{(aq)}, adjusted pH to 6, and extracted with DCM three times. The combined organic layers were dried over Na₂SO₄₍ₛ₎, filtered, and concentrated in vacuo to give crude P1.199 (169 mg) as a sticky colorless oil. ¹H NMR (400 MHz, CDCl₃), δ: 6.14-6.12 (m, 1H), 4.44-4.39 (m, 1H), 3.85 (dd, J=10.4, 5.6 Hz, 1H), 3.77 (s, 2H), 3.66-3.57 (m, 1H), 2.28-2.26 (m, 3H), 1.70 (d, J=7.4 Hz, 1H), 1.54 (dd, J=7.4, 5.2 Hz, 1H), 1.08-1.06 (m, 3H), 0.94-0.91 (m, 3H).

### Preparation 204. (1R,2S,5S)—N—((S)-1-Amino-3-(6-methyl-2-oxo-1,2-dihydroquinolin-3-yl)-1-oxopropan-2-yl)-6,6-dimethyl-3-(2-(3-methylisoxazol-5-yl)acetyl)-3-azabicyclo[3.1.0]hexane-2-carboxamide (P1.200)

To a solution of P1.199 (150.0 mg, 0.501 mmol, 1.0 eq, 93% purity) in DMF (3.6 mL) was added P1.168 (148.0 mg, 0.601 mmol, 1.2 eq), HATU (209.7 mg, 0.551 mmol, 1.1 eq) and DIEA (0.26 mL, 1.50 mmol, 3.0 eq). The reaction mixture was stirred at rt for 16 h. The solution was concentrated and purified by C-18 reversed-phase column chromatography (mobile phase A: water (with 0.1% NH₄HCO₃), mobile phase B: acetonitrile, UV: 214 and 254 nm, Flow rate: 40 mL/min, Gradient: 3-90% (% B)) to give P1.200 (144 mg, 57% yield) as a light-yellow solid. ¹H NMR (400 MHz, CDCl₃), δ: 9.44-9.42 (m, 1H), 8.98 (s, 1H), 7.66 (s, 1H), 7.34-7.28 (m, 2H), 7.17 (d, J=8.4 Hz, 1H), 6.73 (s, 1H), 6.07 (s, 1H), 5.02 (s, 1H), 4.61-4.56 (m, 1H), 4.31-4.23 (m, 3H), 3.73-3.63 (m, 2H), 3.18-3.06 (m, 2H), 2.39 (s, 3H), 2.13 (s, 3H), 1.70 (d, J=7.6 Hz, 1H), 1.58-1.54 (m, 1H), 1.09 (s, 3H), 0.96 (s, 3H).

### Synthesis of (1R,2S,5S)—N—((S)-1-amino-3-(6-methyl-2-oxo-1,2-dihydroquinolin-3-yl)-1-oxopropan-2-yl)-6,6-dimethyl-3-(2-(3-(trifluoromethyl)isoxazol-5-yl)acetyl)-3-azabicyclo[3.1.0]hexane-2-carboxamide (P1.204)

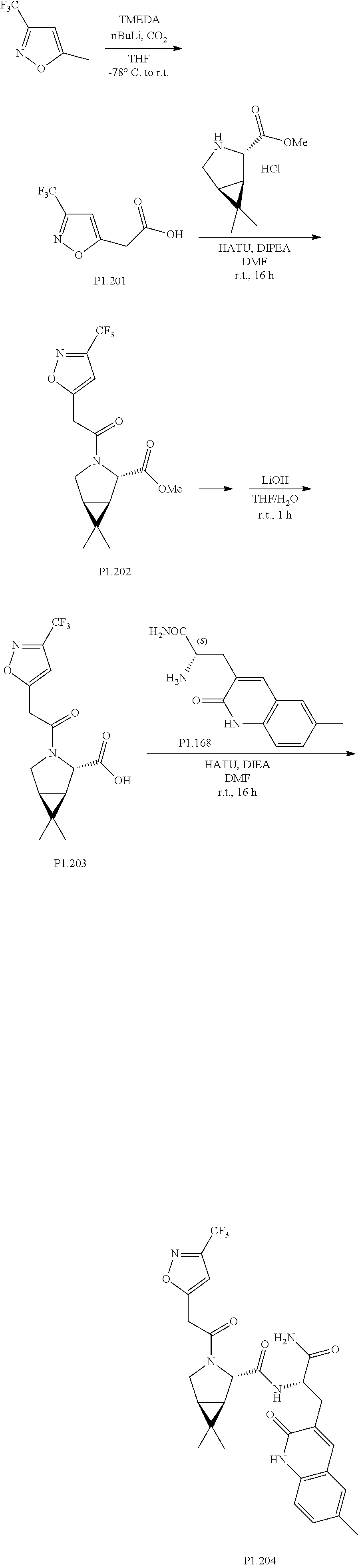

### Preparation 205. 2-(3-(Trifluoromethyl)isoxazol-5-yl)acetic acid (P1.201)

To a solution of 5-methyl-3-(trifluoromethyl)isoxazole (710 mg, 4.70 mmol, 1.0 eq) and (TMEDA, 98 mg, 0.85 mmol, 0.18 eq) in anh. THF (12.3 ml) was added n-butyllithium (1.6 M in hexane, 3.2 mL, 5.17 mmol, 1.1 eq) at −78° C. After the mixture was stirred at −78° C. for 45 min, the mixture was added dry ice at −78° C. The mixture was allowed to warm up to rt and stirred for 16 h. The mixture was treated with water (17.4 mL) and NH₄Cl_{(aq)} (8.7 mL) and then extracted with EtOAc. The water layer was adjusted pH value to 3 with 1N HCl_{(aq)} and extracted with EtOAc. The combined organic layers were dried over Na₂SO₄, filtered, and concentrated under reduced pressure to give P1.201 (483 mg, crude) as a white solid. ¹H NMR (400 MHz, DMSO-d₆), δ: 7.01 (s, 1H), 4.07 (s, 2H).

### Preparation 206. Methyl (1R,2S,5S)-6,6-dimethyl-3-(2-(3-(trifluoromethyl)isoxazol-5-yl)acetyl)-3-azabicyclo[3.1.0]hexane-2-carboxylate (P1.202)

A solution of P1.201 (483 mg, 2.48 mmol, 1.0 eq), (1R,2S,5S)-6,6-Dimethyl-3-aza-bicyclo[3.1.0]hexane-2-carboxylic acid methyl ester hydrochloride (612 mg, 2.98 mmol, 1.2 eq), DIPEA (1.3 mL, 7.44 mmol, 3 eq) and HATU (1.04 g, 2.73 mmol, 1.1 eq) in DMF (24 ml) was stirred at rt for 16 h. The mixture was treated with water slowly and extracted with EtOAc. The combined organic layers were washed with brine, dried over Na₂SO₄, filtered, and concentrated. The mixture was concentrated and purified by silica gel column chromatography (0-80% EtOAc in n-hexane) to give the desired product P1.202 (877 mg, 99%) as a yellow oil. ¹H NMR (400 MHz, CDCl₃), δ: 6.66 (s, 1H), 4.42 (s, 1H), 3.92-3.88 (m, 1H), 3.81-3.80 (m, 1H), 3.77 (s, 3H), 3.72-3.69 (m, 1H), 3.57-3.54 (m, 1H), 1.56-1.47 (m, 2H), 1.07 (s, 3H), 0.96 (s, 3H); LCMS (ESI) m/z calc. for C₁₅H₁₇F₃N₂O₄ 346.11. found, 347.0 [M+H]⁺.

### Preparation 207. (1R,2S,5S)-6,6-Dimethyl-3-(2-(3-(trifluoromethyl)isoxazol-5-yl)acetyl)-3-azabicyclo[3.1.0]hexane-2-carboxylic acid (P1.203)

To a solution of P1.202 (150 mg, 0.43 mmol, 1.0 eq) in THF (2.2 mL) was added lithium hydroxide (311 mg, 1.30 mmol, 3.0 eq) and water (2.2 mL). After the reaction mixture was stirred at rt for 16 h, it was neutralized by 1 M HCl_{(aq)} and then concentrated to give P1.203 (120 mg, crude) as a yellow oil which was used in next step without purification. ¹H NMR (400 MHz, CDCl₃), δ: 6.65 (s, 1H), 4.43 (s, 1H), 3.96-3.80 (m, 3H), 3.59-3.58 (m, 1H), 1.70-1.52 (m, 2H), 1.09 (s, 3H), 0.97 (s, 3H); LCMS (ESI) m/z calc. for C₁₄H₁₅F₃N₂O₄ 332.10. found, 332.9 [M+H]⁺.

### Preparation 208. (1R,2S,5S)—N—((S)-1-Amino-3-(6-methyl-2-oxo-1,2-dihydroquinolin-3-yl)-1-oxopropan-2-yl)-6,6-dimethyl-3-(2-(3-(trifluoromethyl)isoxazol-5-yl)acetyl)-3-azabicyclo[3.1.0]hexane-2-carboxamide (P1.204)

To a solution of P1.203 (120.0 mg, 0.361 mmol, 1.0 eq) in DMF (2.6 mL) was added P1.168 (106.3 mg, 0.433 mmol, 1.2 eq), HATU (151.1 mg, 0.397 mmol, 1.1 eq) and DIEA (0.19 mL, 1.08 mmol, 3.0 eq). The reaction mixture was stirred at rt for 16 h. The solution was concentrated and purified by C-18 reversed-phase column chromatography (mobile phase A: water (with 0.1% NH₄HCO₃), mobile phase B: acetonitrile, UV: 214 and 254 nm, Flow rate: 40 mL/min, Gradient: 3-90% (% B)) to give P1.204 (93 mg, 46% yield) as a white solid. ¹H NMR (400 MHz, CDCl₃), δ: 9.31 (d, J=5.6 Hz, 1H), 9.11 (s, 1H), 7.70 (s, 1H), 7.34-7.29 (m, 2H), 7.07 (d, J=8.0 Hz, 1H), 6.70 (br. s, 1H), 6.51 (s, 1H), 5.18 (s, 1H), 4.63-4.56 (m, 1H), 4.38 (d, J=18.0 Hz, 1H), 4.31-4.24 (m, 2H), 3.85 (d, J=18.0 Hz, 1H), 3.62 (d, J=10.4 Hz, 1H), 3.21-3.05 (m, 2H), 2.39 (s, 3H), 1.69 (d, J=7.6 Hz, 1H), 1.59-1.55 (m, 1H), 1.09 (s, 3H), 0.96 (s, 3H).

### Synthesis of (1R,2S,5S)—N—((S)-1-amino-3-(6-methyl-2-oxo-1,2-dihydroquinolin-3-yl)-1-oxopropan-2-yl)-6,6-dimethyl-3-(2-(3-(trifluoromethyl)isoxazol-5-yl)acetyl)-3-azabicyclo[3.1.0]hexane-2-carboxamide (P1.212)

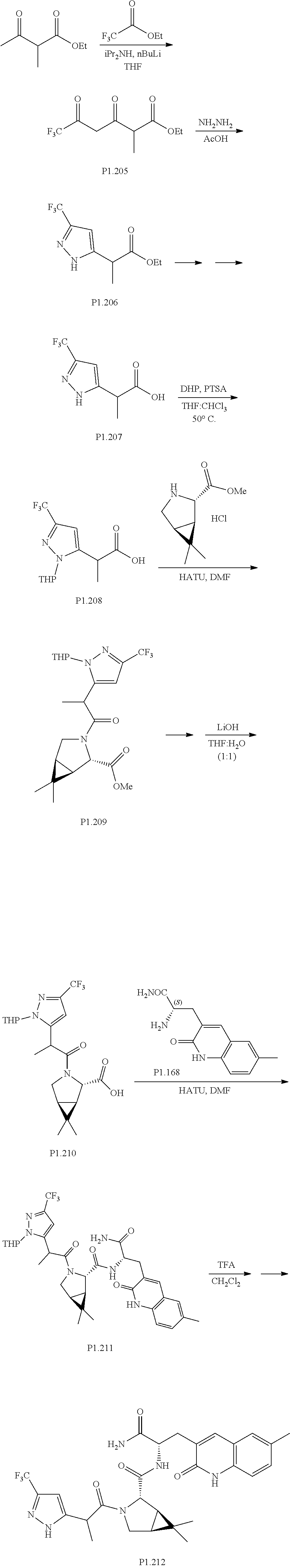

### Preparation 209. 2-[2-Tetrahydropyran-2-yl-5-(trifluoromethyl)pyrazol-3-yl]propanoic acid (P1.208)

To a solution of methyl 2-(3-(trifluoromethyl)-1H-pyrazol-5-yl) propanoic acid (P1.207, 875 mg, 4.2 mmol, 1.0 eq) in THF and CHCl₃ was added dihydropyran (DHP, 766 μL, 8.4 mmol, 2 eq) and p-toluene sulfonic acid (PTSA, 119.8 mg, 0.63 mmol, 0.15 eq) slowly at rt and the reaction mixture was stirred at 50° C. for 48 h. After 48 h, dihydropyran (DHP, 383 μL, 4.2 mmol, 1 eq) and p-toluene sulfonic acid (PTSA, 50 mg, 0.63 mmol, 0.06 eq) were added to the solution slowly at rt and the reaction mixture was stirred at 50° C. for another 12 h. The mixture was concentrated to remove the solvent. The residue was purified by C-18 reversed phase column chromatography (mobile phase A: water, mobile phase B: methanol, UV: 220 and 254 nm, Flow rate: 110 mL/min, Gradient: 60-80% (% B)) to give P1.208 (800 mg, 65% yield). ¹H NMR (400 MHz, DMSO-d₆), δ: 6.70 (s, 1H), 5.58-5.51 (m, 1H), 4.1-4.0 (m, 1H), 3.90-3.77 (m, 1H), 3.70-3.54 (m, 1H), 2.25-2.13 (m, 1H), 1.98-1.85 (m, 2H), 1.66-1.58 (m, 1H), 1.54-1.43 (m, 5H).

### Preparation 210. Methyl (1R,2S,5S)-6,6-dimethyl-3-[2-[2-tetrahydropyran-2-yl-5-(trifluoromethyl)pyrazol-3-yl]propanoyl]-3-azabicyclo[3.1.0]hexane-2-carboxylate (P1.209)

To a solution of P1.208 (300 mg, 1.026 mmol, 1.0 eq) in dry DMF was added methyl (1R,2S,5S)-6,6-dimethyl-3-azabicyclo[3.1.0]hexane-2-carboxylate hydrochloride (253 mg, 1.23 mmol, 1.2 eq), HATU (429.7 mg, 1.13 mmol, 1.1 eq) and DIPEA (536 μL, 3.08 mmol, 3.0 eq) in one portion at rt. The reaction mixture was stirred at rt for 16 h. The mixture was directly purified with C-18 reversed-phase column chromatography (mobile phase A: water, mobile phase B: methanol, UV: 220 and 254 nm, Flow rate: 35 mL/min, Gradient: 70-80% (% B)) to give P1.209 (200 mg, 44% yield) as a white solid. ¹H NMR (400 MHz, DMSO-d₆), δ: 6.58-6.52 (m, 1H), 5.67-5.38 (m, 1H), 4.27-4.13 (m, 2H), 3.90-3.66 (m, 3H), 3.63-3.67 (m, 4H), 2.24-2.17 (m, 1H), 2.00-1.80 (m, 2H), 1.59-1.50 (m, 3H), 1.47-1.28 (m, 5H), 1.01-0.69 (m, 6H).

### Preparation 211. (1R,2S,5S)-6,6-Dimethyl-3-[2-[2-tetrahydropyran-2-yl-5-(trifluoromethyl)pyrazol-3-yl]propanoyl]-3-azabicyclo[3.1.0]hexane-2-carboxylic acid (P1.210)

To a solution of P1.209 (200 mg, 0.451 mmol, 1.0 eq) in dry THF (2.5 mL) was added solution of LiOH in water and stirred the resulting mixture at rt for 2 h. The resulting mixture was diluted with methanol and neutralized with H+ resin. Then the solution was filtered and concentrated to give P1.210 (170 mg, 89% yield) as a white solid. ¹H NMR (400 MHz, DMSO-d₆), δ: 6.68-6.28 (m, 1H), 5.92-5.47 (m, 1H), 4.11-3.57 (m, 4H), 2.21-2.12 (m, 1H), 2.04-1.64 (m, 2H), 1.55-1.38 (m, 3H), 1.35-1.15 (m, 5H), 0.99-0.49 (m, 7H).

### Preparation 212. (1R,2S,5S)—N—((S)-1-Amino-3-(6-methyl-2-oxo-1,2-dihydroquinolin-3-yl)-1-oxopropan-2-yl)-6,6-dimethyl-3-(2-(1-(tetrahydro-2H-pyran-2-yl)-3-(trifluoromethyl)-1H-pyrazol-5-yl)propanoyl)-3-azabicyclo[3.1.0]hexane-2-carboxamide (P1.211)

To a solution of P1.210 (170 mg, 0.40 mmol, 1.0 eq) in dry DMF (3 mL) was added (S)-2-amino-3-(6-methyl-2-oxo-1,2-dihydroquinolin-3-yl)propanamide (118 mg, 0.48 mmol, 1.2 eq), HATU (167 mg, 0.44 mmol, 1.1 eq) and DIPEA (209 μL, 1.2 mmol, 3.0 eq) slowly at rt. The reaction mixture was stirred at rt for 3 h. The reaction mixture was concentrated to get the crude product. The crude was directly purified by C-18 reversed-phase column chromatography (mobile phase A: water, mobile phase B: methanol, UV: 214 and 254 nm, Flow rate: 35 mL/min, Gradient: 70-80% (% B)) to give P1.211 (120 mg, 45% yield) as a white solid. ¹H NMR (400 MHz, CDCl₃-d), δ: 10.34-8.88 (m, 2H), 7.67-7.45 (m, 1H), 7.45-6.15 (m, 5H), 5.58-5.53 (m, 2H), 4.68-4.08 (m, 3H), 3.64-3.94 (m, 3H), 3.41-2.76 (m, 2H), 2.35-2.26 (m, 3H), 2.09-1.38 (m, 11H), 1.08-0.58 (m, 7H).

### Preparation 213. (1R,2S,5S)—N-[(1S)-2-amino-1-[(6-methyl-2-oxo-1H-quinolin-3-yl)methyl]-2-oxo-ethyl]-6,6-dimethyl-3-[2-[3-(trifluoromethyl)-1H-pyrazol-5-yl]propanoyl]-3-azabicyclo[3.1.0]hexane-2-carboxamide (P1.212)

To a solution of P1.211 (120 mg, 0.183 mmol, 1.0 eq) in DCM (4.0 mL) was added TFA (0.90 mL) at rt. The resulting mixture was stirred at rt for 1 h. The mixture was neutralized by basic resin. The solution was filtered and concentrated in vacuo to give P1.212 (70 mg, 72% yield) as a white solid. ¹H NMR (400 MHz, CDCl₃-d), δ: 12.12 (s, 1H), 11.14 (s, 1H), 7.75-7.12 (m, 4H), 4.77-4.51 (m, 2H), 4.40-3.94 (m, 3H), 3.80-3.45 (m, 3H), 3.31-3.02 (m, 3H), 2.39-2.34 (m, 3H), 1.60-1.48 (m, 4H), 1.37-1.25 (m, 3H), 1.08-0.54 (m, 6H).

### Synthesis of (1R,2S,5S)—N—((S)-1-amino-3-(7-methoxy-6-methyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.220)

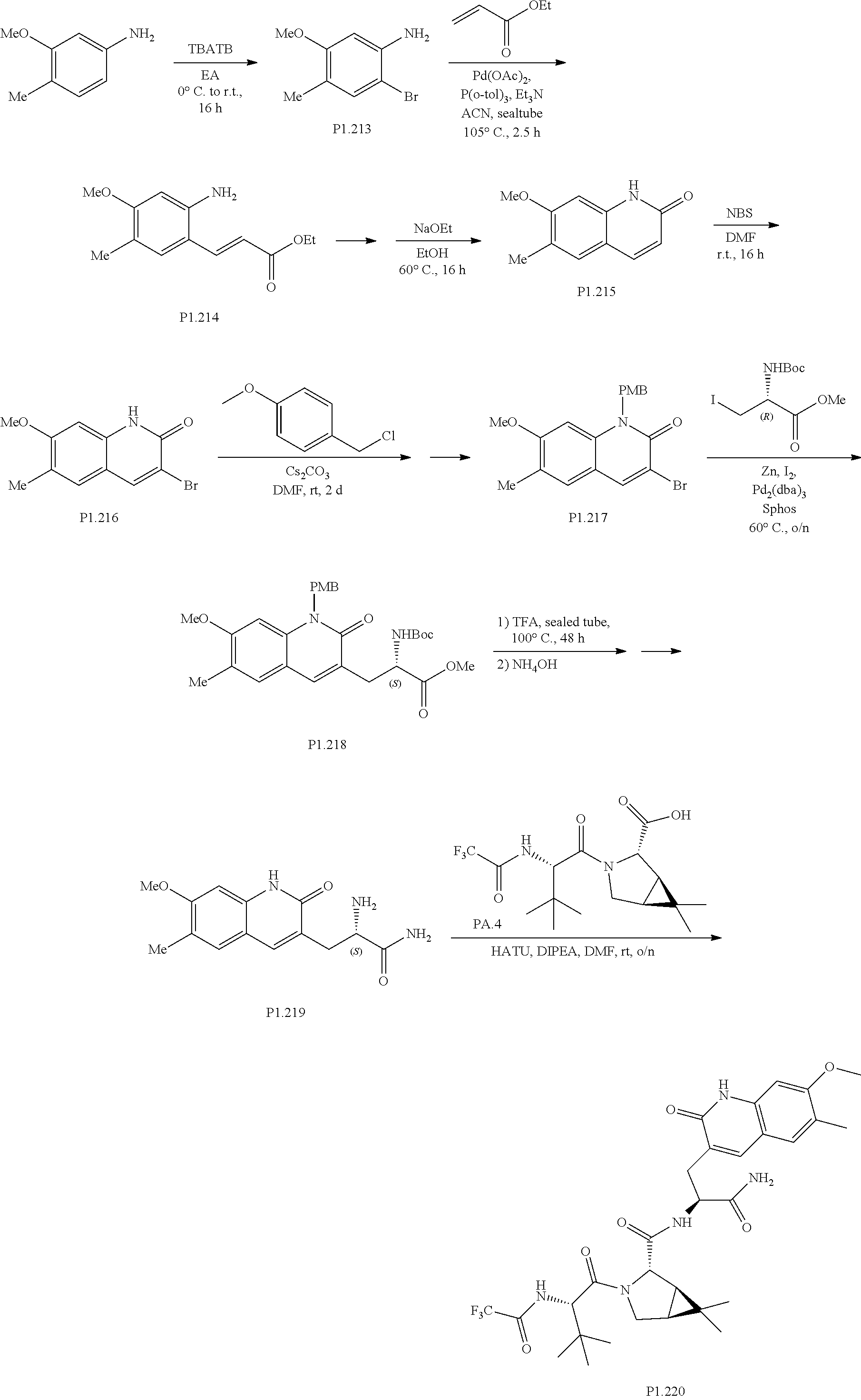

### Preparation 214. 2-Bromo-5-methoxy-4-methylaniline (P1.213)

To a solution of 3-methoxy-4-methyl-aniline (20.0 g, 145.7 mmol) in EtOAc (728 mL) was added tetrabutylammonium tribromide (73.8 g, 153.08 mmol) at 0° C. After stirring at rt for 16 h, the reaction mixture was quenched with NaHCO_{3(aq)} and extracted with EtOAc. The combined organic layers were dried over Na₂SO₄₍ₛ₎, filtered, and concentrated under reduced pressure. The residue was purified by silica gel chromatography (n-hexane:EtOAc=3:1 to 1:1) to give P1.213 (28.5 g, 91% yield) as a yellow solid. ¹H NMR (400 MHz, CDCl₃), δ: 7.12 (s, 1H), 6.28 (s, 1H), 3.93 (br. s, 2H), 3.76 (s, 3H), 2.08 (s, 3H).

### Preparation 215. Ethyl (E)-3-(2-amino-4-methoxy-5-methylphenyl)acrylate (P1.214)

To a solution of P1.213 (15.0 g, 69.4 mmol) in acetonitrile (43 mL) was added ethyl acrylate (9.0 mL, 83.3 mmol), tris(2-methylphenyl)phosphane (1.5 g, 6.9 mmol,) and triethylamine (41 mL, 298.5 mmol) in a sealed tube. The reaction was degassed by argon for 10 min. Palladium(II) acetate (Pd(OAc)₂, 2.5 g, 8.3 mmol) was added to the reaction. The mixture was stirred at 105° C. for 2.5 h. The reaction was cooled to rt, treated with water, and extracted with EtOAc. The combined organic layers were washed with NH₄Cl_{(aq)}, dried over Na₂SO₄₍ₛ₎, filtered, and concentrated in vacuo. The residue was purified by silica gel chromatography (n-hexane:EtOAc=3:1 to 1:1) to give P1.214 (10.5 g, 64% yield) as a yellow solid. ¹H NMR (400 MHz, CDCl₃), δ: 7.76 (d, J=15.6 Hz, 1H), 7.18 (s, 1H), 6.22 (d, J=15.6 Hz, 1H), 6.15 (s, 1H), 4.24 (q, J=7.2 Hz, 2H), 3.94 (br. s, 2H), 3.80 (s, 3H), 2.09 (s, 3H), 1.32 (t, J=7.2 Hz, 3H).

### Preparation 216. 7-Methoxy-6-methylquinolin-2(1H)-one (P1.215)

To a solution of P1.214 (10.5 g, 44.6 mmol) in ethanol (89 mL) was added sodium ethoxide (21% in ethanol, 66.6 mL, 178.5 mmol) at rt. After stirring at 60° C. for 16 h, the mixture was quenched with EtOAc and concentrated. The residue was purified by silica gel chromatography (0-10% MeOH in dichloromethane) to give P1.215 (3.9 g, 46% yield). ¹H NMR (400 MHz, CD₃OD), δ: 7.84 (d, J=9.2 Hz, 1H), 7.39 (s, 1H), 6.83 (s, 1H), 6.43 (d, J=9.2 Hz, 1H), 3.92 (s, 3H), 2.23 (s, 3H).

### Preparation 217. 3-Bromo-7-methoxy-6-methylquinolin-2(1H)-one (P1.216)

To a solution of P1.215 (3.8 g, 20.1 mmol) in DMF (50 mL) was added 1-bromo-2,5-pyrolidinedione (3.9 g, 22.1 mmol) at rt. After the solution was stirred at rt for 16 h, the mixture was diluted with water. The resulting precipitate was collected by filtration and washed with water to give P1.216 (4.43 g, 82% yield). ¹H NMR (400 MHz, CD₃OD), δ: 8.26 (s, 1H), 7.38 (s, 1H), 6.82 (s, 1H), 3.92 (s, 3H), 2.23 (s, 3H).

### Preparation 218. 3-Bromo-7-methoxy-1-(4-methoxybenzyl)-6-methylquinolin-2(1H)-one (P1.217)

To a solution of P1.216 (4.43 g, 16.5 mmol) in DMF (137 mL) was added cesium carbonate (10.1 g, 33.0 mmol) and 4-methoxybenzyl chloride (3.4 mL, 24.8 mmol) at rt. After the reaction was stirred at rt for overnight, the mixture was added cesium carbonate (5.0 g, 16.3 mmol) and 4-methoxybenzyl chloride (2 mL, 14.7 mmol) again at rt. After the reaction mixture was stirred at rt for overnight, the reaction mixture was diluted with EtOAc and washed with water. The organic layer was dried over Na₂SO₄₍ₛ₎, filtered, and concentrated in vacuo. The residue was purified by silica gel chromatography (n-hexane:EtOAc=3:1 to 0:1). The residue was washed with ether and filtered to give P1.217 (4.63 g, 72% yield). ¹H NMR (400 MHz, CDCl₃), δ: 8.03 (s, 1H), 7.22 (d, J=8.8 Hz, 3H), 6.83 (d, J=8.8 Hz, 2H), 6.68 (s, 1H), 5.53 (s, 2H), 3.76 (s, 3H), 3.75 (s, 3H), 2.19 (s, 3H).

### Preparation 219. Methyl (S)-2-((tert-butoxycarbonyl)amino)-3-(7-methoxy-1-(4-methoxybenzyl)-6-methyl-2-oxo-1,2-dihydroquinolin-3-yl)propanoate (P1.218)

To a mixture of zinc powder (4.65 g, 71.12 mmol) in anh. DMF (120 mL) was added iodine (902 mg, 3.55 mmol), and it was stirred at rt. After the color of solution was changed from brown to colorless, methyl (R)-2-((tert-butoxycarbonyl)amino)-3-iodopropanoate (7.80 g, 23.70 mmol) and iodine (902 mg, 3.55 mmol) was added to the reaction mixture. After the color of solution was changed to colorless, it was stirred at rt for another 30 min. To the reaction mixture was added dicyclohexyl(2′,6′-dimethoxy[1,1′-biphenyl]-2-yl)phosphane (SPhos, 486 mg, 1.18 mmol) and P1.127 (4.60 g, 11.85 mmol). The reaction was degassed by argon for 10 min. To the reaction mixture was added tris(dibenzylideneacetone)dipalladium(0) (Pd₂dba₃, 325 mg, 0.35 mmol). The reaction was stirred at 60° C. for 16 h. After cooling to rt, the reaction was diluted with water. The solution was filtered and the filtrate was extracted with EtOAc. The combined organic layers were dried over Na₂SO₄₍ₛ₎, filtered, and concentrated in vacuo to give the residue which was purified by silica gel column chromatography (0-50% EtOAc in n-hexane) to give P1.218 (3.96 g, 65% yield) as a light-yellow solid. ¹H NMR (400 MHz, CDCl₃), δ: 7.50 (s, 1H), 7.21-7.18 (m, 3H), 6.83 (d, J=8.8 Hz, 2H), 6.64 (s, 1H), 6.07 (d, J=6.8 Hz, 1H), 5.63-5.37 (m, 2H), 4.60-4.58 (m, 1H), 3.76 (s, 3H), 3.73 (s, 3H), 3.70 (s, 3H), 3.18-3.04 (m, 2H), 2.19 (s, 3H), 1.38 (s, 9H).

### Preparation 220. (S)-2-Amino-3-(7-methoxy-6-methyl-2-oxo-1,2-dihydroquinolin-3-yl)propanamide (P1.219)

The solution of P1.218 (3.36 g, 6.521 mmol) in TFA (26 mL) was stirred at 100° C. for 48 h in a sealed tube. The reaction mixture was diluted with ethyl acetate and evaporated under reduced pressure. The pH value of the resulting mixture was adjusted to neutral by adding an ammonium hydroxide solution. The crude reaction mixture was purified by silica gel column chromatography by using 0-20% MeOH (containing 1% NH₄OH solution) in DCM to obtain P1.219 as a white solid (322 mg, 18% yield). ¹H NMR (400 MHz, DMSO-d₆), δ: 11.62 (br. s, 1H), 7.60 (s, 1H), 7.51 (br. s, 1H), 7.33 (s, 1H), 7.13 (br. s, 1H), 6.79 (s, 1H), 4.85 (br. s, 1H), 3.81 (s, 3H), 3.64-3.62 (m, 1H), 2.87-2.84 (m, 1H), 2.64-2.61 (m, 1H), 2.13 (s, 3H).

### Preparation 221. (1R,2S,5S)—N—((S)-1-amino-3-(7-methoxy-6-methyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.220)

To a solution of PA.4 (150 mg, 0.412 mmol) in DMF (2.0 mL) was added P1.219 (136.0 mg, 0.494 mmol), HATU (172.3 mg, 0.453 mmol), and DIPEA (0.215 mL, 1.236 mmol). The resulting mixture was stirred at rt overnight. The reaction mixture was extracted with ethyl acetate. The combined organic layers were dried over Na₂SO₄₍ₛ₎, filtered, and concentrated to obtain crude product P1.220 as a yellow solid (159 mg, 62%). ¹H NMR (400 MHz, CD₃OD), δ: 7.76 (s, 1H), 7.39 (s, 1H), 6.80 (s, 1H), 4.58-4.55 (m, 2H), 4.19-4.44 (m, 2H), 3.91 (s, 3H), 3.75 (d, J=10.8 Hz, 1H), 3.09-3.00 (m, 2H), 2.22 (s, 3H), 1.58-1.54 (m, 1H), 1.31 (d, J=7.6 Hz, 1H), 0.99-0.98 (m, 12H), 0.87-0.85 (m, 3H).

### Synthesis of (1R,2S,5S)—N—((S)-1-amino-3-(7-fluoro-6-methyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.228)

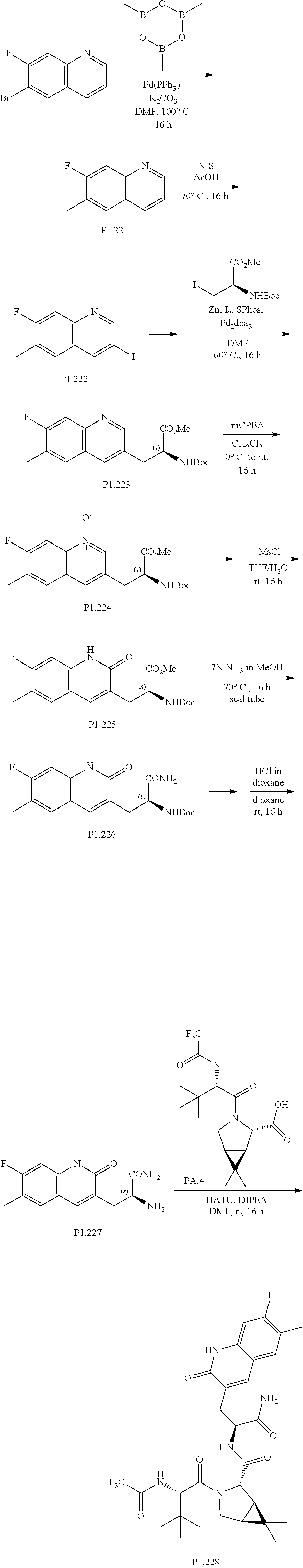

### Preparation 222. 7-Fluoro-6-methylquinoline (P1.221)

A solution of 6-bromo-7-fluoroquinoline (5 g, 22.12 mmol, 1 eq) and potassium carbonate (9.15 g, 66.35 mmol, 3 eq) in DMF (89 ml) was degassed by argon for 15 min. The mixture was added tetrakis(triphenylphosphine)-palladium(0) (2.56 g, 2.20 mmol, 0.1 eq) and then degassed by argon. The mixture was stirred at 100° C. for 16 h. The mixture was cooled to rt and filtrated with a pad of celite. The filtrate was treated with water and extracted with EtOAc for three times. The combined organic layers were washed with brine, dried over Na₂SO₄, filtered, concentrated in vacuo. The residue was purified by silica gel chromatography (0-40% EtOAc in n-hexane) to give P1.221 (3.2 g, 89% yield) as a yellow solid. ¹H NMR (400 MHz, CDCl₃), δ: 8.84 (d, J=4.0 Hz, 1H), 8.05 (d, J=8.2 Hz, 1H), 7.67 (d, J=10.9 Hz, 1H), 7.60 (d, J=8.2 Hz, 1H), 7.34-7.26 (m, 1H), 2.46 (s, 3H); LCMS (ESI) m/z calc. for C₁₀H₈FN 161.06. found, 161.8 [M+H]⁺.

### Preparation 223. 7-Fluoro-3-iodo-6-methylquinoline (P1.222)

To a solution of P1.221 (3.2 g, 19.85 mmol, 1 eq) in AcOH (79 ml) was added N-iodosuccinimide (8.9 g, 39.71 mmol, 2 eq.) and the reaction was stirred at 70° C. for 16 h. The mixture was concentrated, adjusted pH value to 8 with NaHCO_{3(aq)}, and extracted with EtOAc. The combined organic layers were dried over Na₂SO₄, filtered, concentrated under reduced pressure and then purified by silica gel column chromatography (0-60% EtOAc in n-hexane) to give the desired product P1.222 (2.9 g, 50% yield) as a yellow solid. ¹H NMR (400 MHz, CDCl₃), δ: 8.95 (d, J=1.8 Hz, 1H), 8.42 (d, J=1.8 Hz, 1H), 7.63 (d, J=10.8 Hz, 1H), 7.53-7.46 (m, 1H), 2.47-2.46 (m, 3H); LCMS (ESI) m/z calc. for C₁₀H₇FIN 286.96. found, 288.1 [M+H]⁺.

### Preparation 224. Methyl (S)-2-((tert-butoxycarbonyl)amino)-3-(7-fluoro-6-methylquinolin-3-yl)propanoate (P1.223)

To a solution of zinc powder (4.4 g, 60.60 mmol, 6.0 eq.) in anh. DMF (92 mL) was added iodine (770 mg, 3.03 mmol, 0.3 eq.), and it was stirred at rt. After the color of solution was changed from brown to colorless, added methyl (R)-2-((tert-butoxycarbonyl)amino)-3-iodopropanoate (6.6 g, 20.20 mmol, 2.0 eq.) and iodine (770 mg, 3.03 mmol, 0.3 eq.) to the reaction mixture. After the color of solution was changed to colorless again, it was stirred at rt for another 30 min. To the reaction mixture was added dicyclohexyl(2′,6′-dimethoxy[1,1′-biphenyl]-2-yl)phosphane (SPhos, 415 mg, 1.01 mmol, 0.1 eq.) and P1.222 (2.9 g, 10.10 mmol, 1.0 eq), and then degassed by argon for 10 min. To the reaction mixture was added tris(dibenzylideneacetone)dipalladium(0) (Pd₂dba₃, 277 mg, 0.30 mmol, 0.03 eq). The reaction was stirred at 60° C. for overnight. The reaction was treated and diluted with water and filtered. The filtrate was extracted with EtOAc. The combined organic layers were dried over Na₂SO₄₍ₛ₎, filtered, and concentrated in vacuo to give the residue, which was purified by silica gel column chromatography (0-70% EtOAc in n-hexane) to give P1.223 (2.3 g, 62% yield) as a yellow solid. ¹H NMR (400 MHz, CDCl₃), δ: 8.61 (s, 1H), 7.82-7.81 (m, 1H), 7.64 (d, J=10.8 Hz, 1H), 7.55 (d, J=8.1 Hz, 1H), 5.14-5.12 (m, 1H), 4.67-4.65 (m, 1H), 3.73 (s, 3H), 3.34-3.29 (m, 1H), 3.22-3.17 (m, 1H), 2.45 (s, 3H), 1.40 (s, 9H); LCMS (ESI) m/z calc. for C₁₉H₂₃FN₂O₄ 362.16. found, 363.0 [M+H]⁺.

### Preparation 225. (S)-3-(2-((tert-Butoxycarbonyl)amino)-3-methoxy-3-oxopropyl)-7-fluoro-6-methylquinoline 1-oxide (P1.224)

To a solution of P1.223 (2.3 g, 6.35 mmol, 1.0 eq) in DCM (16 mL) was added the solution of 3-chloroperoxybenzoic acid in DCM (16 mL) and stirred at rt for 16 h. The reaction was treated and diluted with NaHCO_{3(aq)} and extracted with EtOAc. The combined organic layers were dried over Na₂SO₄₍ₛ₎, filtered, and concentrated in vacuo to give the residue, which was purified by silica gel column chromatography (0-10% methanol in DCM) to give P1.224 (2.37 g, 98% yield) as an orange solid. ¹H NMR (400 MHz, CDCl₃), δ: 8.44 (s, 1H), 8.27 (d, J=10.4 Hz, 1H), 7.63-7.61 (d, J=7.4 Hz, 1H), 7.52 (s, 1H), 5.41-5.39 (m, 1H), 4.65-4.64 (m, 1H), 3.75 (s, 3H), 3.27-3.10 (m, 2H), 2.45 (s, 3H), 1.38 (s, 9H).; LCMS (ESI) m/z calc. for C₁₉H₂₃FN₂O₅ 378.16. found, 379.1 [M+H]⁺.

### Preparation 226. Methyl (S)-2-((tert-butoxycarbonyl)amino)-3-(7-fluoro-6-methyl-2-oxo-1,2-dihydroquinolin-3-yl)propanoate (P1.225)

A solution of P1.224 (500 mg, 1.32 mmol, 1.0 eq.) and methanesulfonyl chloride in THF (7 mL) and water (7 mL) was stirred at rt for 16 h. The reaction was treated with water and extracted with EtOAc. The combined organic layers were dried over Na₂SO₄₍ₛ₎, filtered, and concentrated in vacuo to give the residue, which was purified by silica gel column chromatography (0-60% EtOAc in n-hexane) to give P1.225 (219 mg, 43% yield) as a yellow solid. ¹H NMR (400 MHz, CDCl₃), δ: 12.51 (br. s, 1H), 7.61 (s, 1H), 7.32 (d, J=7.6 Hz, 1H), 7.09 (d, J=10.0 Hz, 1H), 6.20 (s, 1H), 4.65-4.50 (m, 1H), 3.72 (s, 3H), 3.19-3.05 (m, 2H), 2.32 (s, 3H), 1.35 (s, 9H); LCMS (ESI) m/z calc. for C₁₉H₂₃FN₂O₅ 378.16. found, 379.0 [M+H]⁺.

### Preparation 227. tert-Butyl (S)-(1-amino-3-(7-fluoro-6-methyl-2-oxo-1,2-dihydroquinolin-3-yl)-1-oxopropan-2-yl)carbamate (P1.226)

A solution of P1.225 (200 mg, 0.53 mmol, 1.0 eq) in 7N ammonia in methanol (2 mL) was stirred at 70° C. for 16 h. The solvent was removed under reduced pressure to give the residue which was purified by silica gel column chromatography (0-10% methanol in DCM) to give P1.226 (200 mg, 99% yield) as a yellow solid. ¹H NMR (400 MHz, DMSO-d₆), δ: 11.78 (br. s, 1H), 7.61 (s, 1H), 7.49 (d, J=8.0 Hz, 1H), 7.22 (s, 1H), 6.99-6.96 (m, 2H), 6.87 (d, J=8.8 Hz, 1H), 4.17-4.11 (m, 1H), 2.91-2.89 (m, 1H), 2.72-2.54 (m, 1H), 2.24 (s, 3H), 1.24 (s, 9H); LCMS (ESI) m/z calc. for C₁₈H₂₂FN₃O₄ 363.16. found, 364.0 [M+H]⁺.

### Preparation 228. (S)-2-Amino-3-(7-fluoro-6-methyl-2-oxo-1,2-dihydroquinolin-3-yl)propanamide (P1.227)

To a solution of P1.226 (200 mg, 0.55 mmol, 1.0 eq) in dioxane (3.0 mL) was added hydrogen chloride in 1,4-dioxane (4.0 M, 0.7 mL). The reaction was stirred at rt for 16 h. The solvent was removed under reduced pressure to give P1.227 (140 mg, crude) as a yellow solid which was used in next step without further purification. ¹H NMR (400 MHz, DMSO-d₆), δ: 12.01 (br. s, 1H), 8.23 (s, 2H), 7.93 (s, 1H), 7.75 (s, 1H), 7.61-7.48 (m, 2H), 7.08-7.05 (m, 1H), 4.08-3.98 (m, 1H), 3.01-2.88 (m, 2H), 2.25 (s, 3H); LCMS (ESI) m/z calc. for C₁₃H₁₄FN₃O₂ 263.11. found, 263.9 [M+H]⁺.

### Preparation 229. (1R,2S,5S)—N—((S)-1-amino-3-(7-fluoro-6-methyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.228)

A solution of P1.227 (140 mg, 0.53 mmol, 1.2 eq), PA.4 (160 mg, 0.44 mmol, 1.0 eq), HATU (184 mg, 0.48 mmol), and DIPEA (230 μL, 3.0 eq) in DMF (3 mL) was stirred at rt for 16 h.

The mixture was treated with water and extracted with EtOAc three times. The combined organic layers were washed with brine, dried over Na₂SO₄₍ₛ₎, filtered, and concentrated in vacuo to give the residue. The residue was purified by silica gel column chromatography (0-10% methanol in DCM). The mixture was purified by C-18 reversed-phase column chromatography (0-10% methanol in water) to give P1.228 (165 mg, 61% yield) as a yellow solid. ¹H NMR (400 MHz, CD₃OD), δ: 7.81 (s, 1H), 7.52 (d, J=7.6 Hz, 1H), 7.00 (d, J=10.4 Hz, 1H), 4.62-4.58 (m, 1H), 4.56 (s, 1H), 4.20 (s, 1H), 4.14-4.09 (m, 1H), 3.76 (d, J=10.4 Hz, 1H), 3.17-2.94 (m, 2H), 2.31-2.30 (m, 3H), 1.57-1.53 (m, 1H), 1.31 (d, J=8.2 Hz, 1H), 0.98-0.96 (m, 12H), 0.87 (s, 3H); LCMS (ESI) m/z calc. for C₂₉H₃₅F₄N₅O₅ 609.26. found, 610.1 [M+H]⁺.

### Synthesis of (1R,2S,5S)—N—((S)-1-Amino-3-(6,7-difluoro-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.236)

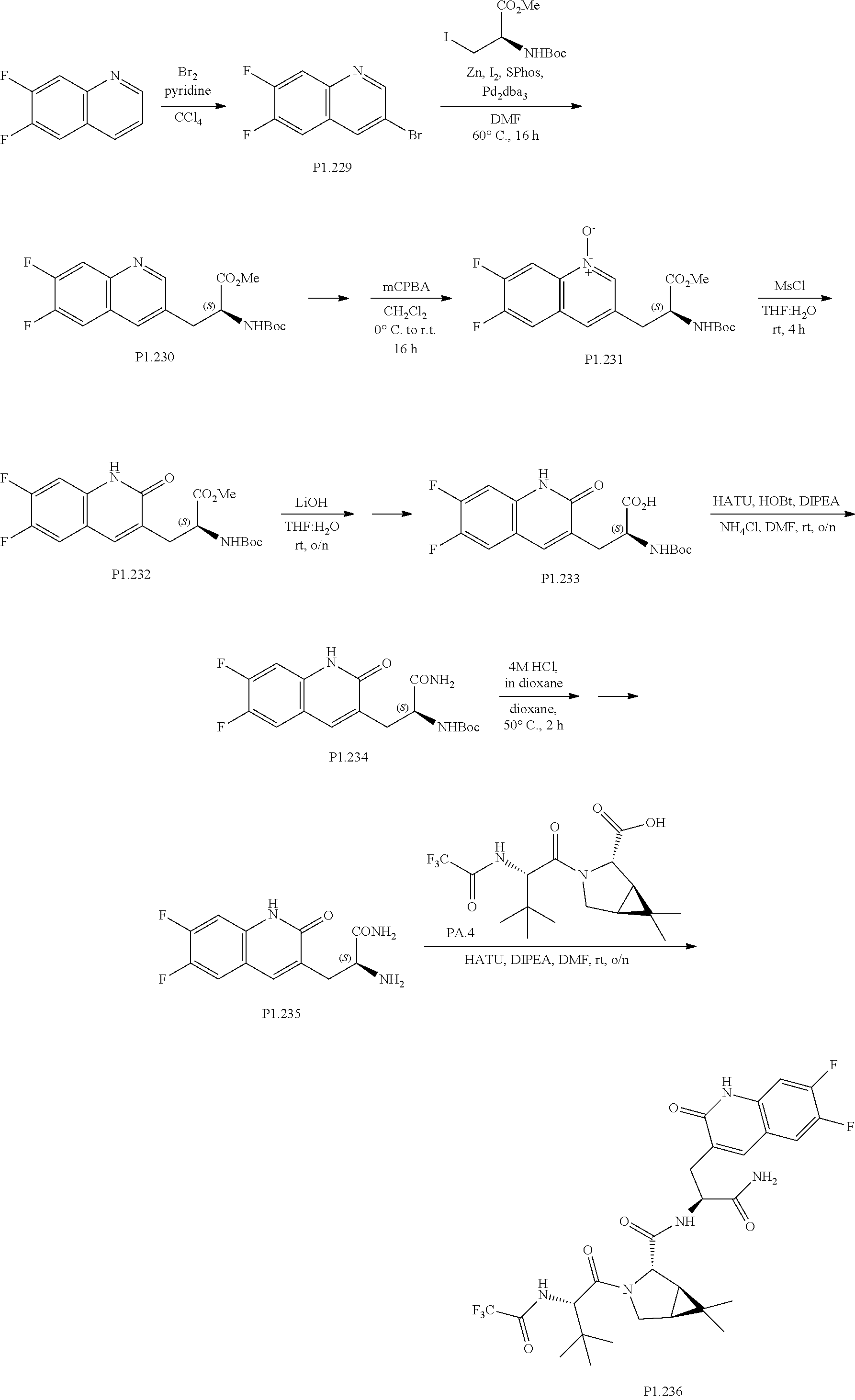

### Preparation 230. 3-Bromo-6,7-difluoroquinoline (P1.229)

To a solution of 6,7-difluoroquinoline (1.00 g, 6.37 mmol, 1.0 eq) in tetrachloromethane (13 mL) was added bromine (0.748 mL, 15.93 mmole, 2.5 eq) and pyridine (1.1 mL, 15.93 mmole, 2.5 eq) at 0° C., and stirred at rt for 48 h. Then mixture was treated with water and extracted with DCM. The combined organic layers were dried over Na₂SO₄, filtered, and concentrated. The residue was purified by silica gel column chromatography (0-50% EtOAc in n-hexane) to give P1.229 (1.023 g, 69% yield) as a solid. ¹H NMR (400 MHz, CDCl₃), δ: 8.88 (d, J=2.0, 1H), 8.26 (d, J=2.0, 1H), 7.86-7.81 (m, 1H), 7.51-7.46 (m, 1H).

### Preparation 231. (S)-2-((tert-Butoxycarbonyl)amino)-3-(6,7-difluoroquinolin-3-yl)propanoate (P1.230)

To a mixture of zinc powder (1.64 g, 25.08 mmol, 6.0 eq) in anh. DMF (42 mL) was added iodine (320 mg, 1.25 mmol, 0.3 eq), and it was stirred at rt. After the color of solution was changed from brown to colorless, the solution was added methyl (R)-2-((tert-butoxycarbonyl)amino)-3-iodopropanoate (2.75 g, 8.36 mmol, 2.0 eq) and iodine (320 mg, 1.25 mmol, 0.3 eq) to the reaction mixture. After the color of the solution was changed to colorless, it was stirred at rt for another 30 min. To the reaction mixture was added dicyclohexyl(2′,6′-dimethoxy[1,1′-biphenyl]-2-yl)phosphane (SPhos, 172 mg, 0.42 mmol, 0.1 eq) and P1.229 (1.02 g, 4.18 mmol, 1.0 eq), and then degassed by argon for 10 min. To the reaction mixture was added tris(dibenzylideneacetone)dipalladium(0) (Pd₂dba₃, 115 mg, 0.13 mmol, 0.03 eq), then stirred at 60° C. for overnight. The reaction was treated with water and filtered. The filtrate was extracted with EtOAc. The combined organic layers were dried over Na₂SO₄₍ₛ₎, filtered, and concentrated in vacuo to give the residue, which was purified by silica gel column chromatography (0-60% EtOAc in n-hexane) to give P1.230 (990 mg, 65% yield) as a white solid. ¹H NMR (400 MHz, CDCl₃), δ: 8.66 (s, 1H), 7.87-7.80 (m, 2H), 7.52-7.47 (m, 1H), 5.11 (br. d, 1H), 4.70-4.65 (m, 1H), 3.75 (s, 3H), 3.38-3.33 (m, 1H), 3.23-3.18 (m, 1H), 1.40 (s, 9H).

### Preparation 232. (S)-3-(2-((tert-butoxycarbonyl)amino)-3-methoxy-3-oxopropyl)-6,7-difluoroquinoline 1-oxide (P1.231)

To the reaction mixture of m-CPBA (908.3 mg, 4.05 mmol) in DCM (10 mL) was added solution of P1.230 in DCM (990 mg, 2.702 mmol). The reaction was stirred overnight at rt. After completion of reaction, the reaction mixture was treated with NaHCO_{3(aq.)} and extracted with DCM. The combined organic layers were dried over Na₂SO₄₍ₛ₎, filtered, and concentrated to obtain a residue. The residue was purified by silica gel column chromatography (0-20% DCM in methanol) to give P1.231 as a white solid (990 mg, 96%). ¹H NMR (400 MHz, CDCl₃), δ: 8.53-8.46 (m, 1H), 8.43 (s, 1H), 7.59-7.51 (m, 2H), 5.27 (d, J=7.2 Hz, 1H), 4.67-4.65 (m, 1H), 3.77 (s, 3H), 3.30-3.27 (m, 1H), 3.15-3.11 (m, 1H), 1.40 (s, 9H).

### Preparation 233. Methyl (S)-2-((tert-butoxycarbonyl)amino)-3-(6,7-difluoro-2-oxo-1,2-dihydroquinolin-3-yl)propanoate (P1.232)

To a solution of P1.231 (990 mg, 2.589 mmol) in THF (13 mL) and H₂O (13 mL) was added MsCl (0.401 mL, 5.178 mmol) and then the resulting mixture was stirred at rt for 4 h. After completion of reaction, the reaction mixture was diluted with water and extracted with DCM. The combined organic layers were dried over Na₂SO₄₍ₛ₎, filtered and concentrated to obtain to give crude product P1.232 as a light-yellow solid which was directly used in next step without further purification. ¹H NMR (400 MHz, CDCl₃), δ: 11.57 (s, 1H) 7.58 (s, 1H) 7.33-7.28 (m, 1H), 7.18-7.14 (m, 1H), 5.80 (br. d, 1H), 4.60 (br. s, 1H), 3.72 (s, 3H), 3.16-3.14 (m, 1H), 3.03-3.01 (m, 1H), 1.33 (s, 9H).

### Preparation 234. (S)-2-((tert-Butoxycarbonyl)amino)-3-(6,7-difluoro-2-oxo-1,2-dihydroquinolin-3-yl)propanoic acid (P1.233)

To a solution of P1.232 (930 mg, 2.43 mmol) in THF (30.0 mL) was added a solution of LiOH (174.8 mg, 7.29 mmol) in water (30.0 mL). The resulting mixture was stirred at rt for overnight. The solvent was evaporated to obtain brown syrup P1.233 (650 mg, 73%) as a crude product which was used in next step without further purification. ¹H NMR (400 MHz, CD₃OD), δ: 7.75 (s, 1H) 7.57-7.52 (m, 1H), 7.23-7.18 (m, 1H), 4.51-4.48 (m, 1H), 3.26-3.21 (m, 1H), 2.81-2.75 (m, 1H), 1.29 (s, 9H).

### Preparation 235. tert-Butyl (S)-(1-amino-3-(6,7-difluoro-2-oxo-1,2-dihydroquinolin-3-yl)-1-oxopropan-2-yl)carbamate (P1.234)

To a solution of P1.233 (650 mg, 1.765 mmol) in DMF (7 mL) was added HATU (805.3 mg, 2.12 mmol), HOBt (135.2 mg, 0.882 mmol), NH₄Cl (141.6 mg, 2.65 mmol) and DIPEA (1.54 mL, 8.83 mmol). The resulting mixture was stirred at rt for overnight. After completion of reaction. the reaction mixture was quenched by water and extracted with ethyl acetate. The combined organic layers were dried over Na₂SO₄₍ₛ₎, filtered and concentrated to obtain crude residue and it was purified by silica gel column chromatography (0-20% DCM in methanol) to give P1.234 as a yellow solid (432 mg, 67%). ¹H NMR (400 MHz, DMSO-d₆), δ: 11.90 (br. s, 1H), 7.75 (m, 1H) 7.64 (s, 1H), 7.22 (br. s, 2H), 7.01 (br. s, 1H), 6.86 (d, J=8.0 Hz, 1H), 4.16 (m, 1H), 2.96-2.92 (m, 1H), 2.65-2.60 (m, 1H), 1.22 (s, 9H).

### Preparation 236. (S)-2-Amino-3-(6,7-difluoro-2-oxo-1,2-dihydroquinolin-3-yl)propanamide hydrochloride (P1.235)

To a solution of P1.234 (430 mg, 1.70 mmol) in 1,4-dioxane (8.5 mL) was added HCl (1.46 mL, 5.85 mmol, in 1,4-dioxane). The resulting mixture was stirred at rt for overnight. The reaction mixture was concentrated to obtain crude product P1.235 as a white solid (371 mg, quantitative) which was directly used in next step without further purification.

### Preparation 237. (1R,2S,5S)—N—((S)-1-amino-3-(6,7-difluoro-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.236)

To a solution of PA.4 (150 mg, 0.412 mmol) in DMF (2 mL) was added P1.235 (150 mg, 0.494 mmol), HATU (172.3 mg, 0.453 mmol) and DIPEA (0.215 mL, 1.236 mmol). The resulting mixture stirred at rt for overnight. Then reaction mixture was quenched by water and extracted with ethyl acetate. The combined organic layers were dried over Na₂SO_{4(S)}, filtered, and concentrated to obtain crude product P1.236 as a yellow solid (282 mg, quantitative) which was directly used in next step without further purification. ¹H NMR (400 MHz, CD₃OD), δ: 7.83 (s, 1H), 7.61-7.57 (m, 1H), 7.22-7.17 (m, 1H), 4.64-4.61 (m, 1H), 4.59 (s, 1H), 4.55 (s, 1H), 4.23 (s, 1H), 4.10-4.06 (m, 1H), 3.77 (d, J=10.4 Hz, 1H), 3.14-3.10 (m, 1H), 3.00-2.99 (m, 1H), 1.38-1.33 (m, 2H), 1.03 (s, 3H), 0.96 (s, 9H), 0.89 (s, 3H).

### Synthesis of (1R,2S,5S)—N—((S)-1-amino-3-(4,6-dimethyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.243)

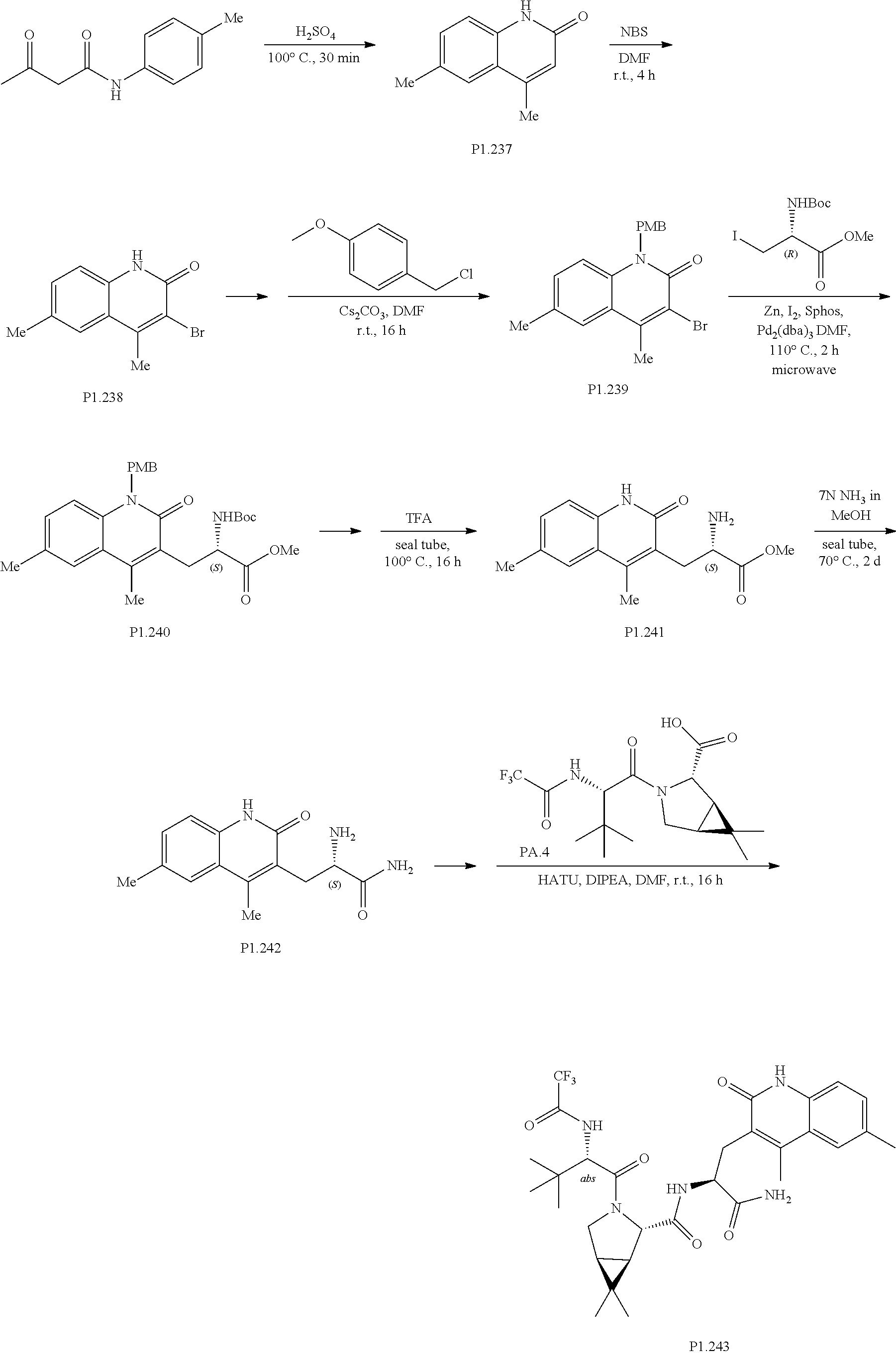

### Preparation 238. 4,6-Dimethylquinolin-2(1H)-one (P1.237)

Added 3-oxo-N-(p-tolyl)butanamide (10 g, 52.3 mmol, 1.0 eq) to concentrated sulfuric acid (18 mL) slowly at rt and the reaction solution was stirred at 100° C. for 30 min. After the reaction solution was cooled to rt, the solution was poured into ice water and neutralized with NaOH_{(aq)} and Na₂CO_{3(aq)}. The resulting precipitate was collected by filtration and washed with water to afford P1.237 (9.1 g, 100%) as a white solid. ¹H NMR (400 MHz, CDCl₃), δ: 12.59 (br. s, 1H), 7.46 (s, 1H), 7.38-7.32 (m, 2H), 6.58 (s, 1H), 2.50 (s, 3H), 3.43 (s, 3H).

### Preparation 239. 3-Bromo-4,6-dimethylquinolin-2(1H)-one (P1.238)

To a solution of P1.237 (4.30 g, 24.8 mmol, 1.0 eq) in DMF (62 mL) was added N-bromosuccinimide (NBS, 4.86 g, 27.3 mmol, 1.1 eq) at rt. After the reaction mixture was stirred at rt for 4 h, the reaction mixture was poured into water. The resulting precipitate was collected by filtration. The solid was treated with diethyl ether to prepare a solution. The solid was collected by filtration and washed with diethyl ether to afford P1.238 (3.15 g, 50%) as a white solid. ¹H NMR (400 MHz, DMSO-d₆), δ: 12.06 (br. s, 1H), 7.62 (s, 1H), 7.37 (d, J=8.4 Hz, 1H), 7.23 (d, J=8.4 Hz, 1H), 2.61 (s, 3H), 2.37 (s, 3H).

### Preparation 240. 3-Bromo-1-(4-methoxybenzyl)-4,6-dimethylquinolin-2(1H)-one (P1.239)

To a solution of P1.238 (3.15 g, 12.5 mmol, 1.0 eq) in DMF (31 mL) was added cesium carbonate (8.14 g, 24.9 mmol, 2.0 eq) and 4-methoxybenzyl chloride (2.53 mL, 18.7 mmol, 1.5 eq) at rt. After the reaction mixture was stirred at rt for 16 h, the reaction mixture was poured into water. The precipitate was collected and washed with water. The solid was purified by silica gel chromatography (n-hexane:EtOAc=3:1) to give a product. The solid was washed with diethyl ether and filtered to afford P1.239 (2.50 g, 54%). ¹H NMR (400 MHz, CDCl₃), δ: 7.56 (s, 1H), 7.30-7.24 (m, 2H), 7.18 (d, J=8.4 Hz, 1H), 6.81 (d, J=8.4 Hz, 1H), 5.54 (br. s, 2H), 3.75 (s, 3H), 2.73 (s, 3H), 2.41 (s, 3H).

### Preparation 241. Methyl (S)-2-((tert-butoxycarbonyl)amino)-3-(1-(4-methoxybenzyl)-4,6-dimethyl-2-oxo-1,2-dihydroquinolin-3-yl)propanoate (P1.240)

To a solution of zinc powder (1.58 g, 24.1 mmol, 6.0 eq) in anh. DMF (7 mL) was added iodine (307 mg, 1.21 mmol, 0.3 eq) and stirred at rt. After the color of the solution was changed from red-brown to colorless, the solution was added methyl (S)-2-((tert-butoxycarbonyl)amino)-3-iodopropanoate (2.65 g, 8.05 mmol, 2.0 eq) and iodine (307 mg, 1.21 mmol, 0.3 eq) and stirred at rt. After the color of the solution was changed from red-brown to colorless again, it was stirred for another 30 min and then added 1.239 (1.50 g, 4.03 mmol, 1.0 eq) and 2-dicyclohexylphosphino-2′,6′-dimethoxybiphenyl (82.7 mg, 0.201 mmol, 0.05 eq) at rt. After the mixture solution was degassed with Argon_{(g)}, tris(dibenzylideneacetone)dipalladium(0) (92.2 mg, 0.101 mmol, 0.025 eq) was added to the reaction mixture. The reaction was stirred under microwave irradiation at 110° C. for 2 h. The reaction solution was diluted with EtOAc and water. The solution was filtered by filter paper and washed with EtOAc. The filtrate was extracted with EtOAc. The combined organic layers were washed with water, dried over MgSO₄₍ₛ₎, filtered and concentrated under reduced pressure. The residue was purified by silica gel chromatography (25% EtOAc in n-hexane) to afford P1.240 (790 mg, 40%) as a yellow solid. ¹H NMR (400 MHz, CDCl₃), δ: 7.55 (s, 1H), 7.24-7.13 (m, 4H), 6.81 (d, J=8.4 Hz, 2H), 6.01 (d, J=7.2 Hz, 1H), 5.59-5.40 (m, 2H), 4.48-4.42 (m, 1H), 3.75 (s, 3H), 3.72 (s, 3H), 3.40-3.20 (m, 2H), 2.56 (s, 3H), 2.39 (s, 3H), 1.37 (s, 9H).

### Preparation 242. Methyl (S)-2-amino-3-(4,6-dimethyl-2-oxo-1,2-dihydroquinolin-3-yl)-propanoate TFA salt (P1.241)

P1.240 (1.58 g, 3.19 mmol, 1.0 eq) was dissolved in anh. TFA (5.0 mL) which was dried by molecular sieve 4 Å and stirred at 100° C. for 16 h. The reaction solution was diluted with EtOAc and concentrated under reduced pressure. The residue was purified by C18 reverse column (5-100% methanol in H₂O containing 0.1% TFA) to afford P1.241 (TFA salt, 430 mg, 35%) as a white solid. ¹H NMR (400 MHz, DMSO-d₆), δ: 11.77 (br. s, 1H), 8.44-8.29 (br. m, 3H), 7.59 (s, 1H), 7.33 (d, J=8.4 Hz, 1H), 7.21 (d, J=8.4 Hz, 1H), 4.17-4.15 (m, 1H), 3.62 (s, 3H), 3.23-3.07 (m, 2H), 2.42 (s, 3H), 2.37 (s, 3H).

### Preparation 243. (S)-2-Amino-3-(4,6-dimethyl-2-oxo-1,2-dihydroquinolin-3-yl)propanamide (P1.242)

P1.241 (TFA salt, 430 mg, 1.11 mmol, 1.0 eq) was treated with a solution of 7 N ammonia in methanol (7 mL) in a sealed tube and stirred at 70° C. for 2 days. The reaction solution was concentrated under reduced pressure to afford P1.242 (357 mg, crude) as a yellow solid. ¹H NMR (400 MHz, DMSO-d₆), δ: 11.77 (br. s, 1H), 7.72 (s, 1H), 7.58 (s, 1H), 7.45 (s, 1H), 7.32 (d, J=8.4 Hz, 1H), 7.23 (d, J=8.4 Hz, 1H), 3.87-3.83 (m, 1H), 3.08-3.05 (m, 1H), 2.40 (s, 3H), 2.37 (s, 3H).

### Preparation 244. (1R,2S,5S)—N—((S)-1-amino-3-(4,6-dimethyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.243)

To a solution of PA.4 (418 mg, 1.14 mmol, 1.0 eq) in DMF (7 mL) was added P1.242 (357 mg, 1.37 mmol, 1.2 eq), HATU (479 mg, 1.26 mmol, 1.1 eq), and DIPEA (599 μL, 3.44 mmol, 3.0 eq) at rt. After the reaction mixture was stirred at rt for 16 h, the solution was diluted with water and extracted with EtOAc. The organic layers were dried over MgSO₄₍ₛ₎, filtered, and concentrated under reduced pressure. The residue was purified by silica gel chromatography (10% methanol in DCM) and followed by C18 reverse column (5-100% methanol in H₂O) to afford P1.243 (312 mg, 45%) as a white solid. ¹H NMR (400 MHz, CDCl₃): δ 9.99 (br. s, 1H), 8.08 (d, J=6.0 Hz, 1H), 7.48 (s, 1H), 7.28 (s, 1H), 7.14 (s, 1H), 7.04-6.97 (m, 2H), 5.65 (s, 1H), 4.61 (d, J=10.0 Hz, 1H), 4.49-4.44 (m, 1H), 4.18-4.14 (m, 2H), 3.81 (d, J=10.0 Hz, 1H), 3.31-3.19 (m, 2H), 2.57 (s, 3H), 2.43 (s, 3H), 1.50-1.47 (m, 1H), 1.30-1.28 (m, 1H), 0.99-0.96 (m, 12H), 0.79 (s, 3H).

### Synthesis of (1R,2S,5S)—N—((S)-1-amino-3-(6-methoxy-7-methyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.250)

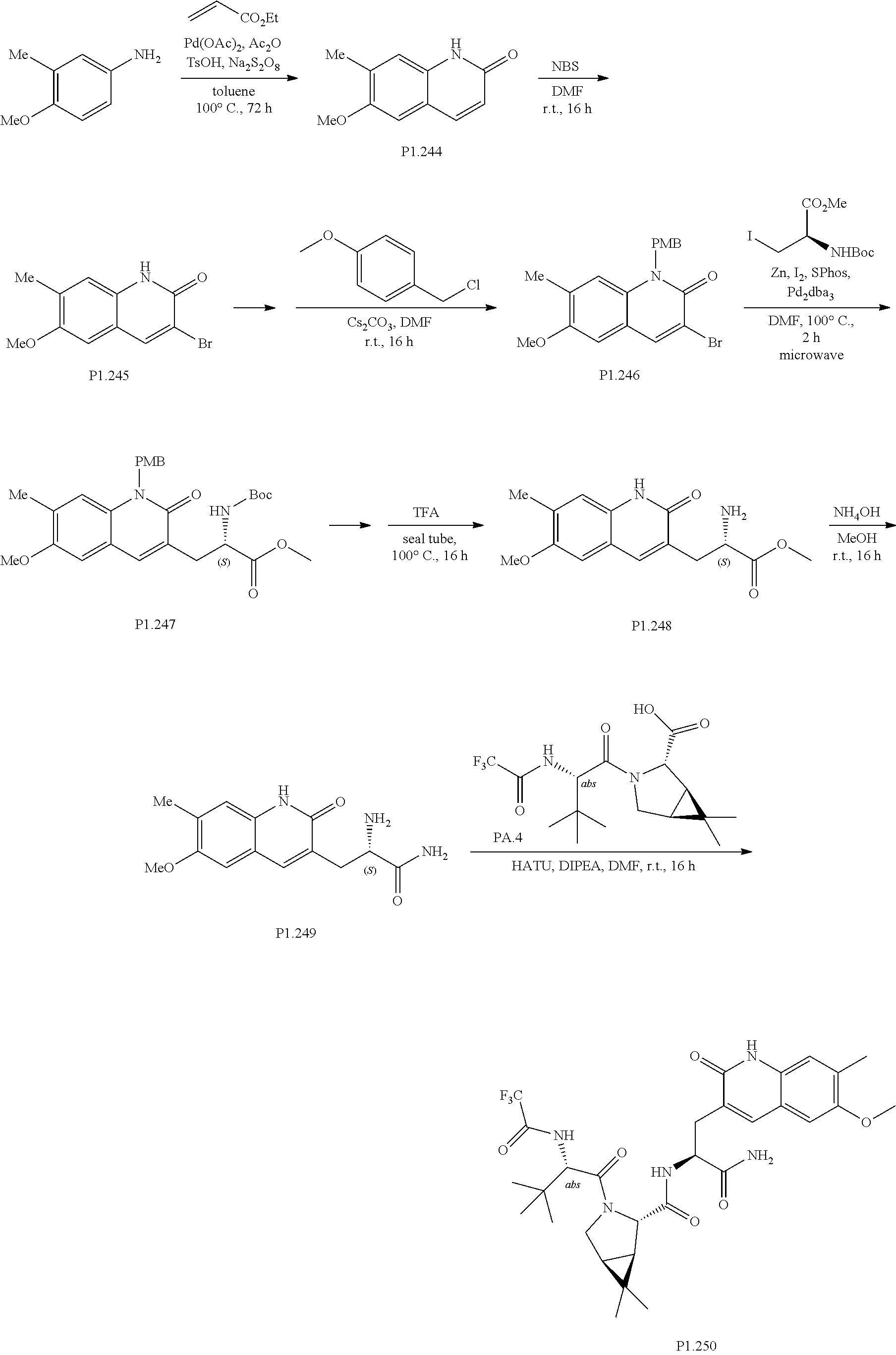

### Preparation 245. 6-Methoxy-7-methylquinolin-2(1H)-one (P1.244)

To a solution of ethyl acrylate (59.0 mL, 546 mmol, 5.2 eq) in toluene (437 mL) was added acetic anhydride (20.0 mL, 218 mmol, 2.1 eq) at rt. After stirring at rt for 10 min to the reaction mixture was added 4-methoxy-3-methyl-aniline (14.2 g, 103 mmol, 1.0 eq), p-toluenesulfonic acid monohydrate (20.8 g, 109 mmol, 1.1 eq), sodium persulfate (78.1 g, 328 mmol, 3.1 eq) and palladium(II) acetate (Pd(OAc)₂, 1.28 g, 5.46 mmol, 0.05 eq). After the reaction was stirred at 100° C. for 72 h, the reaction mixture was filtered through a pad of celite and washed with DCM. The filtrate was concentrated under reduced pressure. The residue was purified by silica gel chromatography (0-10% MeOH in DCM) to afford P1.244 (2.3 g, 12%) as a brown solid. ¹H NMR (400 MHz, CDCl₃), δ: 7.74 (d, J=9.2 Hz, 1H), 7.19 (s, 1H), 6.88 (s, 1H), 6.67 (d, J=9.2 Hz, 1H), 3.88 (s, 3H), 2.33 (s, 3H).

### Preparation 246. Ethyl 3-bromo-6-methoxy-7-methylquinolin-2(1H)-one (P1.245)

To a solution of P1.244 (2.3 g, 12.1 mmol, 1.0 eq) in DMF (30 mL) was added 1-bromo-2,5-pyrolidinedione (2.4 g, 13.4 mmol, 1.1 eq) at rt. After the reaction mixture was stirred at rt for 16 h, the reaction mixture was diluted with water and filtered. The precipitate was collected and washed with water to afford P1.245 (2.0 g, 61%) as a brown solid. ¹H NMR (400 MHz, CD₃OD), δ: 8.35 (s, 1H), 7.16 (s, 1H), 7.08 (s, 1H), 3.88 (s, 3H), 2.29 (s, 3H).

### Preparation 247. 3-Bromo-6-methoxy-1-(4-methoxybenzyl)-7-methylquinolin-2(1H)-one (P1.246)

To a solution of P1.245 (2.0 g, 7.40 mmol, 1.0 eq) in DMF (62 mL) was added cesium carbonate (4.6 g, 14.9 mmol, 2.0 eq) and 4-methoxybenzyl chloride (1.5 mL, 11.2 mmol, 1.5 eq) at rt. After the reaction mixture was stirred at rt for 16 h, the reaction mixture was diluted with EtOAc and washed with water. The organic layer was dried over Na₂SO₄₍ₛ₎, filtered, and concentrated in vacuo. The residue was purified by silica gel chromatography (n-hexane:EtOAc=5:1 to 1:1) to give a crude product. The crude product was washed with diethyl ether and filtered to afford P1.246 (1.2 g, 41%). ¹H NMR (400 MHz, CDCl₃), δ: 8.10 (s, 1H), 7.20 (d, J=8.4 Hz, 2H), 7.13 (s, 1H), 6.87-6.78 (m, 3H), 5.51 (s, 2H), 3.85 (s, 3H), 3.76 (s, 3H), 2.24 (s, 3H).

### Preparation 248. Methyl (S)-2-((tert-butoxycarbonyl)amino)-3-(6-methoxy-1-(4-methoxybenzyl)-7-methyl-2-oxo-1,2-dihydroquinolin-3-yl)propanoate (P1.247)

To a solution of zinc powder (352 mg, 5.39 mmol, 6.0 eq) in anh. DMF (6 mL) was added iodine (68.4 mg, 0.269 mmol, 0.3 eq). The reaction was stirred at rt until the color of the solution was changed from red-brown to colorless. To the reaction mixture was added methyl (S)-2-((tert-butoxycarbonyl)amino)-3-iodopropanoate (591 mg, 1.79 mmol, 2.0 eq) and iodine (68.4 mg, 0.269 mmol, 0.3 eq) and stirred at rt again. After a red-brown solution was stirred at rt until it became colorless, it was stirred for another 30 min and was added P1.246 (349 mg, 0.898 mmol, 1.0 eq) and 2-dicyclohexylphosphino-2′,6′-dimethoxybiphenyl (18.4 mg, 0.0449 mmol, 0.05 eq) at rt. The reaction mixture was degassed with Argon_{(g)}. To the reaction was added tris(dibenzylideneacetone)dipalladium(0) (20.5 mg, 0.0224 mmol, 0.025 eq). The reaction was stirred under microwave at 100° C. for 2 h. The reaction solution was diluted with EtOAc and water. The solution was filtered off and washed with EtOAc. The filtrate was extracted with EtOAc and washed with water. The organic layers were dried over MgSO₄₍ₛ₎, filtered, and concentrated under reduced pressure. The residue was purified by silica gel chromatography (25% EtOAc in n-hexane) to afford P1.247 (435 mg, 95%) as a yellow solid. ¹H NMR (400 MHz, CDCl₃), δ: 7.57 (s, 1H), 7.16 (d, J=8.8 Hz, 2H), 7.08 (s, 1H), 6.84-6.81 (m, 3H), 6.07-6.06 (m, 1H), 5.59-5.37 (br., 2H), 4.61-4.56 (m, 1H), 3.84 (s, 3H), 3.76 (s, 3H), 3.70 (s, 3H), 3.20-3.06 (m, 2H), 2.24 (s, 3H), 1.38 (s, 9H).

### Preparation 249. Methyl (S)-2-amino-3-(6-methoxy-7-methyl-2-oxo-1,2-dihydroquinolin-3-yl)propanoate TFA salt (P1.248)

P1.247 (720 mg, 1.41 mmol, 1.0 eq) was dissolved in anh. TFA (5 mL) which was dried by molecular sieve 4 Å and the reaction was stirred at 100° C. for 16 h. The reaction solution was diluted with EtOAc and concentrated under reduced pressure. The residue was purified by C18 flash column (5-100% methanol in H₂O containing 0.1% TFA) to afford P1.248 (325 mg, 88%) as a white solid. ¹H NMR (400 MHz, DMSO-d₆), δ: 11.86 (br. s, 1H), 8.45 (br. s, 3H), 7.75 (s, 1H), 7.11 (s, 2H), 4.37 (s, 1H), 3.81 (s, 3H), 3.68 (s, 3H), 3.09-2.93 (m, 2H), 2.22 (s, 3H).

### Preparation 250. (S)-2-Amino-3-(6-methoxy-7-methyl-2-oxo-1,2-dihydroquinolin-3-yl)propanamide (P1.249)

To a solution of P1.248 (TFA salt, 325 mg, 0.803 mmol, 1.0 eq) in methanol (10 mL) was added 30% ammonium hydroxide solution (5.0 mL). After the solution was stirred at rt for 16 h, the reaction solution was concentrated and treated with acetonitrile. The resulting precipitate was collected by filtration and washed with acetonitrile to afford P1.249 (167 mg, 75%) as a white solid. ¹H NMR (400 MHz, DMSO-d₆): δ 11.85 (br. s, 1H), 7.83 (br. s, 1H), 7.70 (s, 1H), 7.49 (br. s, 1H), 7.10-7.05 (m, 2H), 4.01-3.98 (m, 1H), 3.78 (s, 3H), 2.99-2.85 (m, 2H), 2.19 (s, 3H).

### Preparation 251. (1R,2S,5S)—N—((S)-1-amino-3-(6-methoxy-7-methyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.250)

To a solution of PA.4 (202 mg, 0.553 mmol, 1.0 eq) in DMF (3.0 ml) was added P1.249 (183 mg, 0.664 mol, 1.2 eq), HATU (231 mg, 0.609 mmol, 1.1 eq) and DIPEA (289 μl, 1.66 mmol, 3.0 eq) at rt. After the reaction mixture was stirred at rt for 16 h, the reaction was purified by C18 flash column (5-100% methanol in H₂O) to afford P1.250 (107 mg, 31%) as a yellow solid. ¹H NMR (400 MHz, CDCl₃), δ: 10.00 (br. s, 1H), 8.55 (br. s, 1H), 7.69 (s, 1H), 7.12 (br. s, 1H), 6.93 (br. s, 1H), 6.80-6.79 (m, 2H), 5.51 (br. s, 1H), 4.64-4.61 (m, 2H), 4.30 (s, 1H), 4.19-4.15 (m, 1H), 3.84-3.81 (m, 4H), 3.26-3.08 (m, 2H), 2.24 (s, 3H), 1.57-1.38 (m, 2H), 1.02-0.81 (m, 15H).

### Synthesis of (1R,2S,5S)—N—((S)-1-amino-3-(6-methyl-2-oxo-1,2-dihydroquinolin-3-yl)-1-oxopropan-2-yl)-3-((S)-3,3-dimethyl-2-((2,2,2-trifluoroethyl)sulfonamido)butanoyl)-6,6-dimethyl-3-azabicyclo[3.1.0]hexane-2-carboxamide (P1.253)

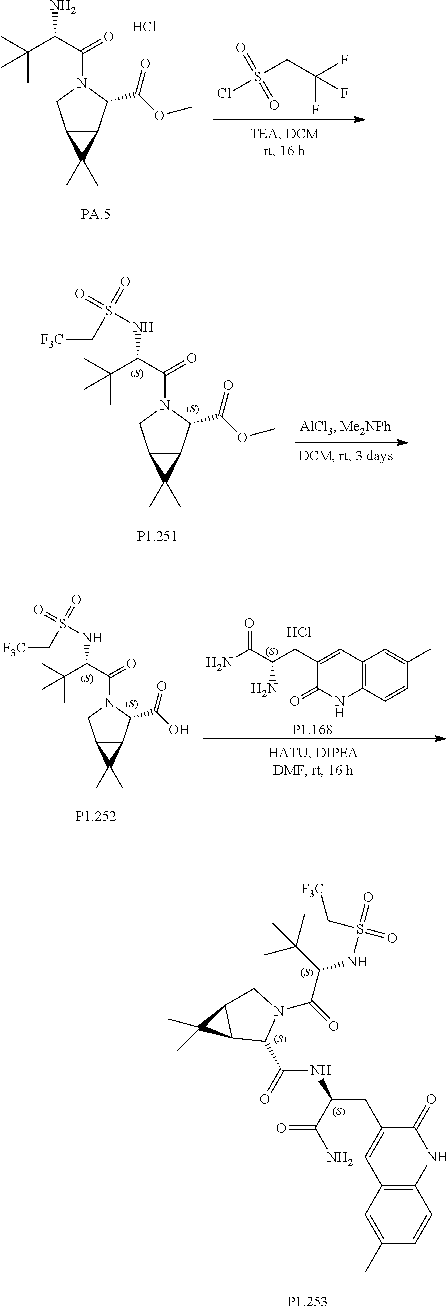

### Preparation 252. Methyl (1R,2S,5S)-3-((S)-3,3-dimethyl-2-((2,2,2-trifluoroethyl)sulfonamido)butanoyl)-6,6-dimethyl-3-azabicyclo[3.1.0]hexane-2-carboxylate (P1.251)

To a solution of PA.5 (6.00 g, 18.9 mmol) in anh. DCM (190 mL) was added triethylamine (7.9 mL, 57 mmol) and 2,2,2-trifluoroethane-1-sulfonyl chloride (3.1 mL, 28 mmol). After the mixture was stirred at rt for 16 h, the reaction was diluted with DCM. The solution was washed with NaHCO_{3(aq)} and brine. The organic layer was dried over MgSO₄, filtered, and concentrated under reduced pressure. The residue was purified by silica gel chromatography (0-5% MeOH in CH₂Cl₂) to give P1.251 (5.03 g, 62%) as a white solid. ¹H NMR (400 MHz, CDCl₃), δ: 5.57 (d, J=9.2 Hz, 1H), 4.50 (s, 1H), 3.95-3.87 (m, 2H), 3.77-3.71 (m, 6H), 1.53-1.45 (m, 2H), 1.07 (s, 9H), 1.06 (s, 3H), 0.96 (s, 3H); LCMS (ESI) m/z calc. for C₁₇H₂₇F₃N₂O₅S 428.16. found, 429.1 [M+H]⁺.

### Preparation 253. (1R,2S,5S)-3-((S)-3,3-Dimethyl-2-((2,2,2-trifluoroethyl)sulfonamido)butanoyl)-6,6-dimethyl-3-azabicyclo[3.1.0]hexane-2-carboxylic acid (P1.252)

To a solution of P1.251 (1.70 g, 3.97 mmol) in anh. DCM (26 mL) was added N,N-dimethylaniline (7.5 mL, 60 mmol) and aluminum chloride (4.76 g, 35.7 mmol). After the mixture was stirred at rt for 3 days, the mixture was diluted with ethyl ether and quenched with H₂O at 0° C. The mixture was washed with 1N HCl. The organic layer was dried over Na₂SO₄₍ₛ₎, filtered, and concentrated under reduced pressure. The residue was purified by reverse phase chromatography (0-100% ACN in H₂O) to give P1.252 (1.04 g, 63%) as a green powder. ¹H NMR (400 MHz, CDCl₃), δ: 5.79 (d, J=9.6 Hz, 1H), 4.50 (s, 1H), 3.97-3.85 (m, 2H), 3.82-3.69 (m, 3H), 1.65 (d, J=6.9 Hz, 1H), 1.59-1.50 (m, 1H), 1.15-0.90 (m, 15H); LCMS (ESI) m/z calc. for C₁₆H₂₅F₃N₂O₅S 414.14. found, 415.1 [M+H]⁺.

### Preparation 254. (1R,2S,5S)—N—((S)-1-Amino-3-(6-methyl-2-oxo-1,2-dihydroquinolin-3-yl)-1-oxopropan-2-yl)-3-((S)-3,3-dimethyl-2-((2,2,2-trifluoroethyl)sulfonamido)butanoyl)-6,6-dimethyl-3-azabicyclo[3.1.0]hexane-2-carboxamide (P1.253)

To a solution of P1.252 (2.20 g, 5.31 mmol) and P1.168 (1.79 g, 6.37 mmol) in DMF (50 mL) was added DIPEA (2.77 mL, 15.9 mmol) and HATU (2.22 g, 5.84 mmol). After the mixture was stirred at rt for 16 h, the mixture was diluted with EtOAc. The solution was washed with H₂O and brine. The organic layer was dried over MgSO₄, filtered, and concentrated under reduced pressure. The residue was purified by silica gel chromatography (0-5% MeOH in DCM containing 1% NH₄OH_{(aq)}) to give P1.253 (2.0 g, 59%) as a white solid. ¹H NMR (400 MHz, DMSO-d₆): δ 11.75 (s, 1H), 8.83-8.31 (m, 1H), 7.80-7.58 (m, 2H), 7.42-6.91 (m, 5H), 4.55-4.29 (m, 2H), 4.20-4.09 (m, 2H), 3.90-3.82 (m, 2H), 3.68-3.65 (m, 1H), 2.98-2.80 (m, 2H), 2.30 (s, 3H), 1.63-1.44 (m, 1H), 1.25-1.15 (m, 1H), 1.04-0.80 (m, 15H); LCMS (ESI) m/z calc. for C₂₉H₃₈F₃N₅O₆S 641.25. found, 642.1 [M+H]⁺.

### Synthesis of (1R,2S,5S)—N—((S)-1-Amino-3-(6-methyl-2-oxo-1,2-dihydroquinolin-3-yl)-1-oxopropan-2-yl)-3-(4-methoxybenzofuran-2-carbonyl)-6,6-dimethyl-3-azabicyclo[3.1.0]hexane-2-carboxamide (P1.258)

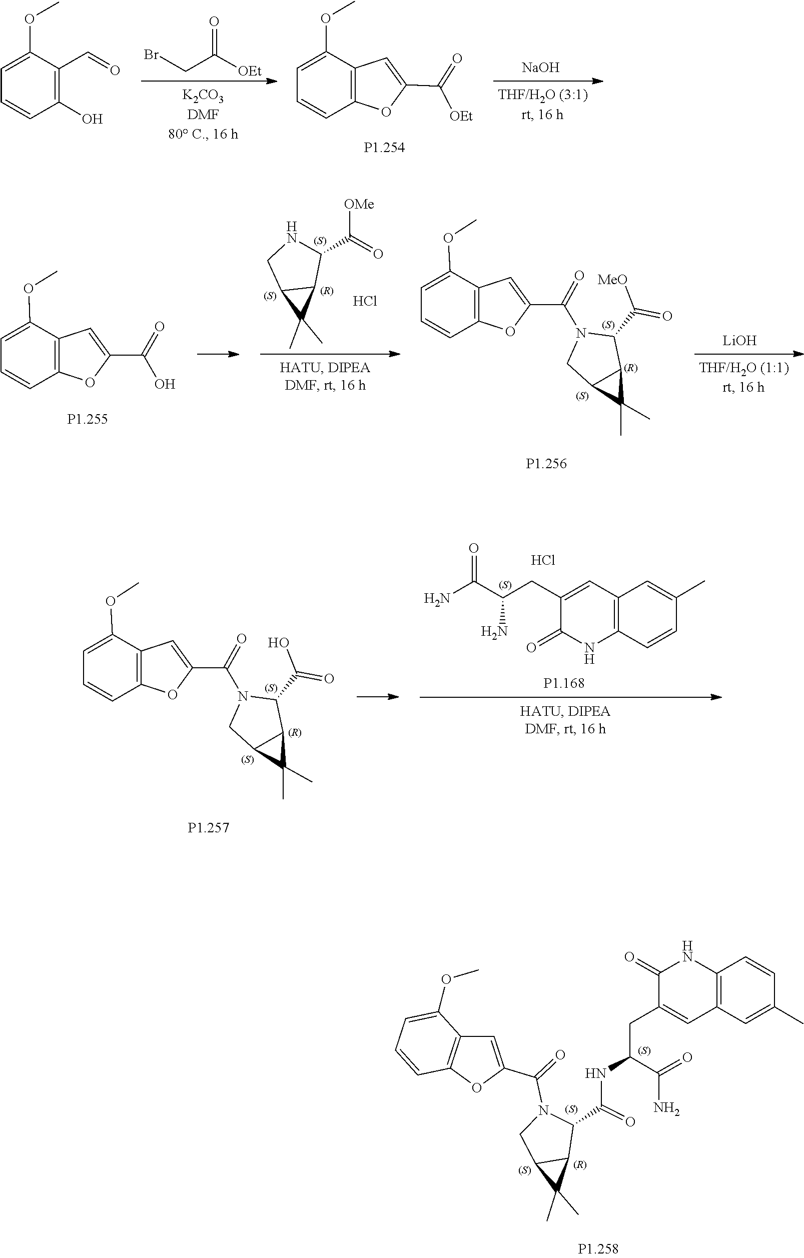

### Preparation 255. Ethyl 4-methoxybenzofuran-2-carboxylate (P1.254)

To a solution of 2-hydroxy-6-methoxy-benzaldehyde (1.00 g, 6.57 mmol) and potassium carbonate (1.09 g, 7.89 mmol) in anh. DMF (33 mL) was added ethyl bromoacetate (0.87 mL, 7.9 mmol) at 0° C. After the mixture was stirred at 80° C. for 16 h, the solution was diluted with DCM. The solution was washed with water and brine. The organic layer was dried over MgSO₄, filtered, and concentrated under reduced pressure. The residue was purified by silica gel chromatography (0-20% DCM in n-hexane) to give P1.254 (1.26 g, 87%) as a white solid. ¹H NMR (400 MHz, CDCl₃), δ: 7.63 (s, 1H), 7.36 (dd, J=8.4, 8.0 Hz, 1H), 7.19 (d, J=8.4 Hz, 1H), 6.68 (d, J=8.0 Hz, 1H), 4.48-4.38 (q, J=7.2 Hz, 2H), 3.95 (s, 3H), 1.42 (t, J=7.2 Hz, 3H); LCMS (ESI) m/z calc. for C₁₂H₁₂ 04220.07. found, 220.9 [M+H]⁺.

### Preparation 256. 4-Methoxybenzofuran-2-carboxylic acid (P1.255)

To a solution of P1.254 (1.20 g, 5.45 mmol) in THF (9 mL) and water (3 mL) was added sodium hydroxide (1.31 g, 32.7 mmol). After the mixture was stirred at rt for 16 h, the mixture was cooled to 0° C., adjusted to pH 2 with 2N HCl_{(aq)} and extracted with DCM. The combined organic layers were dried over MgSO₄, filtered, and concentrated under reduced pressure. Give the desired product P1.255 (995 mg, 95%) as a white solid. ¹H NMR (400 MHz, CDCl₃), δ: 7.78 (s, 1H), 7.46-7.36 (m, 1H), 7.21 (d, J=7.6 Hz, 1H), 6.70 (d, J=8.0 Hz, 1H), 3.97 (s, 3H); LCMS (ESI) m/z calc. for C₁₀H₈O₄ 192.04. found, 192.9 [M+H]⁺.

### Preparation 257. Methyl (1R,2S,5S)-3-(4-methoxybenzofuran-2-carbonyl)-6,6-dimethyl-3-azabicyclo[3.1.0]hexane-2-carboxylate (P1.256)

To a solution of P1.255 (1.00 g, 5.35 mmol) and methyl (1R,2S,5S)-6,6-dimethyl-3-azabicyclo[3.1.0]hexane-2-carboxylate hydrochloride (1.00 g, 4.86 mmol) in DMF (50 mL) was added DIPEA (2.54 mL, 14.6 mmol) and HATU (2.03 g, 5.35 mmol). After the mixture was stirred at rt for 16 h, the mixture was diluted with EtOAc. The solution was washed with H₂O and brine. The organic layer was dried over MgSO₄, filtered, and concentrated under reduced pressure. The residue was purified by silica gel chromatography (0-40% EtOAc in n-hexane) to give P1.256 (2.00 g, 99%) as a white solid. ¹H NMR (400 MHz, CDCl₃), δ: 7.57-7.54 (m, 1H), 7.37-7.28 (m, 1H), 7.18-7.01 (m, 1H), 6.71-6.63 (m, 1H), 5.10-4.62 (m, 1H), 4.32-4.09 (m, 1H), 4.00-3.88 (m, 4H), 3.83-3.70 (m, 3H), 1.67-1.58 (m, 1H), 1.52-1.43 (m, 1H), 1.09 (s, 3H), 1.04-0.97 (m, 3H); LCMS (ESI) m/z calc. for C₁₉H₂₁NO₅ 343.14. found, 344.0 [M+H]⁺.

### Preparation 258. (1R,2S,5S)-3-(4-Methoxybenzofuran-2-carbonyl)-6,6-dimethyl-3-azabicyclo[3.1.0]hexane-2-carboxylic acid (P1.257)

To a solution of P1.256 (2.00 g, 5.82 mmol) in THF (30 mL) and water (30 mL) was added lithium hydroxide (418 mg, 17.5 mmol). After the mixture was stirred at rt for 16 h, the mixture was cooled to 0° C., adjusted to pH 2 with 2N HCl_{(aq)} and extracted with EtOAc. The combined organic layers were dried over MgSO₄, filtered, and concentrated under reduced pressure to give the desired product P1.257 (1.77 g, 92%) as a white solid. ¹H NMR (400 MHz, CDCl₃), δ: 7.62-7.56 (m, 1H), 7.38-7.34 (m, 1H), 7.17-6.98 (m, 1H), 6.71-6.60 (m, 1H), 5.10-4.70 (m, 1H), 4.29-3.86 (m, 5H), 1.84-1.68 (m, 1H), 1.67-1.43 (m, 1H), 1.14-1.06 (m, 3H), 1.03-0.93 (m, 3H); LCMS (ESI) m/z calc. for C₁₈H₁₉NO₅ 329.13. found, 330.0 [M+H]⁺.

### Preparation 259. (1R,2S,5S)—N—((S)-1-Amino-3-(6-methyl-2-oxo-1,2-dihydroquinolin-3-yl)-1-oxopropan-2-yl)-3-(4-methoxybenzofuran-2-carbonyl)-6,6-dimethyl-3-azabicyclo[3.1.0]hexane-2-carboxamide (P1.258)

To a solution of P1.257 (300 mg, 0.911 mmol) and P1.168 (385 mg, 1.37 mmol) in DMF (9 mL) was added DIPEA (0.48 mL, 2.7 mmol) and HATU (381 mg, 1.00 mmol). After the mixture was stirred at rt for 16 h, the mixture was diluted with EtOAc. The solution was washed with H₂O and brine. The organic layer was dried over MgSO₄, filtered, and concentrated under reduced pressure. The residue was purified by silica gel chromatography (0-5% MeOH in DCM containing 1% NH₄OH_{(aq)}) to give P1.258 (472 mg, 93%) as a white solid. ¹H NMR (400 MHz, DMSO-d₆), δ: 11.73-11.65 (m, 1H), 8.64-8.09 (m, 1H), 8.09-7.53 (m, 2H), 7.53-6.90 (m, 7H), 6.90-6.56 (m, 1H), 4.84-4.22 (m, 2H), 3.98-3.69 (m, 4H), 3.12-3.10 (m, 1H), 2.68-2.60 (m, 2H), 2.26 (s, 3H), 1.54-1.21 (m, 2H), 0.99-0.77 (m, 6H); LCMS (ESI) m/z calc. for C₃₁H₃₂N₄O₆ 556.23. found, 557.1 [M+H]⁺.

### Synthesis of benzyl ((2S)-1-((3aR,6aR)-5-(((S)-1-amino-i-oxo-3-(2-oxo-1,2-dihydroquinolin-3-yl)propan-2-yl)carbamoyl)hexahydro-4H-furo[3,2-b]pyrrol-4-yl)-3,3-dimethyl-1-oxobutan-2-yl)carbamate (P1.262)

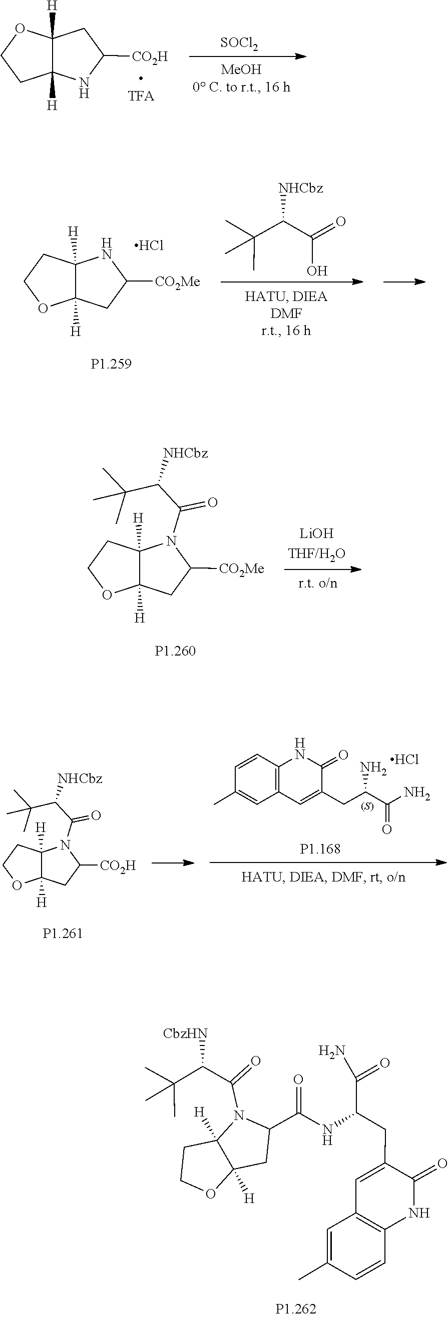

### Preparation 260. Methyl (3aR,6aR)-hexahydro-2H-furo[3,2-b]pyrrole-5-carboxylate hydrochloride (P1.259)

To a solution of (3aR,6aR)-3,3a,4,5,6,6a-hexahydro-2H-furo[3,2-b]pyrrole-5-carboxylic acid; 2,2,2-trifluoroacetaldehyde (900 mg, 3.32 mmol) in methanol (10.0 mL) was added thionyl chloride (0.506 mL, 7.0 mmol) at 0° C. The reaction was stirred at rt for overnight. The solvents were evaporated to obtain desired crude product P1.259 (334 mg, 62% yield) as a brown oil which was directly used in the next step without further purification. LRMS (ESI) m/z 272.1 [M+H]⁺.

### Preparation 261. Methyl (3aR,6aR)-4-((S)-2-(((benzyloxy)carbonyl)amino)-3,3-dimethylbutanoyl)hexahydro-2H-furo[3,2-b]pyrrole-5-carboxylate (P1.260)

To a solution of P1.259 (330 mg, 1.589 mmol) in DMF (11.3 mL) was added (S)-2-(benzyloxy)carbonyl)amino)-3,3-dimethylbutanoic acid (506 mg, 1.91 mmol), HATU (665 mg, 1.75 mmol), and DIPEA (0.830 mL, 4.77 mmol), the resulting mixture was stirred at rt for overnight. The reaction mixture was treated with water and extracted with ethyl acetate. The combined organic layers were dried over Na₂SO₄₍ₛ₎, filtered, and concentrated. The residue was purified by silica gel column chromatography (0-40% ethyl acetate in hexane) to give desired product P1.260 (440 mg, quantitative yield) as a yellow solid. ¹H NMR (400 MHz, CDCl₃), δ: 7.34-7.26 (m, 5H), 5.53-4.95 (m, 4H), 4.72-4.31 (m, 3H), 4.17-3.81 (m, 2H), 3.73-3.56 (m, 4H), 2.65-2.14 (m, 4H), 1.17-0.94 (m, 9H). LRMS (ESI) m/z 419.0 [M+H]⁺.

### Preparation 262. (3aR,6aR)-4-((S)-2-(((Benzyloxy)carbonyl)amino)-3,3-dimethylbutanoyl)hexahydro-2H-furo[3,2-b]pyrrole-5-carboxylic acid (P1.261)

To a solution of P1.260 (440 mg, 1.1 mmol) in THF (9.0 mL) was added a solution of LiOH in water (50.0 mg, 2.082 mmol, 9.0 mL). The resulting mixture was stirred at rt for overnight. The reaction mixture was acidified with 1N HCl (aq.), adjusted pH to ˜6, and extracted with DCM. The combined organic layers were dried over Na₂SO₄₍ₛ₎, filtered, and concentrated to give crude product P1.261 (390 mg, 91%) as a brown solid which was directly used in the next step without further purification. LRMS (ESI) m/z 405.0 [M+H]⁺.

### Preparation 263. Benzyl ((2S)-1-((3aR,6aR)-5-(((S)-1-amino-i-oxo-3-(2-oxo-1,2-dihydroquinolin-3-yl)propan-2-yl)carbamoyl)hexahydro-4H-furo[3,2-b]pyrrol-4-yl)-3,3-dimethyl-1-oxobutan-2-yl)carbamate (P1.262)

To a solution of P1.261 (390 mg, 0.964 mmol) in DMF (7.0 mL) was added P1.168 (326 mg, 1.15 mmol), HATU (403 mg, 1.06 mmol), and DIPEA (0.504 mL, 2.9 mmol). The resulting mixture was stirred at rt for overnight. The reaction mixture was treated with water and extracted with ethyl acetate. The combined organic layers were dried over Na₂SO₄₍ₛ₎, filtered, and concentrated. The residue was purified by silica gel column chromatography (0-20% methanol in dichloromethane) to give the desired product P1.262 (286 mg, 47% yield) as a yellow solid. ¹H NMR (400 MHz, CD₃OD), δ: 7.80-7.71 (m, 1H), 7.42-7.05 (m, 8H), 5.13-5.07 (m, 1H), 4.82-4.26 (m, 6H), 3.95-3.36 (m, 2H), 3.16-2.81 (m, 3H), 2.69-2.48 (m, 1H), 2.38-2.36 (m, 3H), 2.31-2.12 (m, 2H), 1.05-0.99 (m, 9H). LRMS (ESI) m/z 632.1 [M+H]⁺.

### Synthesis of (3aR,6aR)—N—((S)-1-amino-3-(6-methyl-2-oxo-1,2-dihydroquinolin-3-yl)-1-oxopropan-2-yl)-4-((S)-3,3-dimethyl-2-(2,2,2-trifluoroacetamido)butanoyl)hexahydro-2H-furo[3,2-b]pyrrole-5-carboxamide (P1.264)

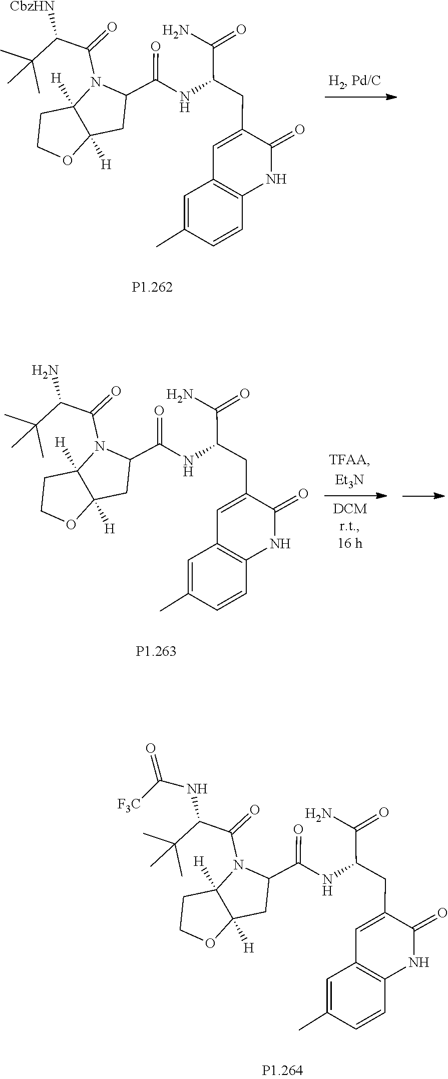 [SMILES: Cc1ccc2[nH]c(=O)c(C[C@H](NC(=O)C3C[C@H]4OCC[C@H]4N3C(=O)[C@@H](NC(=O)OCc3ccccc3)C(C)(C)C)C(N)=O)cc2c1 ; Cc1ccc2[nH]c(=O)c(C[C@H](NC(=O)C3C[C@H]4OCC[C@H]4N3C(=O)[C@@H](N)C(C)(C)C)C(N)=O)cc2c1 ; Cc1ccc2[nH]c(=O)c(C[C@H](NC(=O)C3C[C@H]4OCC[C@H]4N3C(=O)[C@@H](NC(=O)C(F)(F)F)C(C)(C)C)C(N)=O)cc2c1]

### Preparation 264. (3aR,6aR)-4-((S)-2-Amino-3,3-dimethylbutanoyl)-N—((S)-1-amino-3-(6-methyl-2-oxo-1,2-dihydroquinolin-3-yl)-1-oxopropan-2-yl)hexahydro-2H-furo[3,2-b]pyrrole-5-carboxamide (P1.263)

A solution of P1.262 (125 mg, 0.198 mmol) in MeOH (4.0 mL) was stirred at rt for 3 h under H_{2(g)} balloon. The reaction mixture was filtered through Celite and concentrated under reduced pressure to afford P1.263 (88 mg, 89%) as a white solid. ¹H NMR (400 MHz, CDCl₃), δ: 8.96-8.70 (m, 1H), 7.71 (s, 1H), 7.30-7.24 (m, 2H), 7.14-6.86 (m, 3H), 5.40 (s, 1H), 4.75-4.25 (m, 5H), 3.85-3.69 (m, 1H), 3.55 (s, 1H), 3.21-2.98 (m, 3H), 2.67-2.55 (m, 1H), 2.45-2.10 (m, 7H), 1.09-0.85 (m, 9H).

### Preparation 265. (3aR,6aR)—N—((S)-1-amino-3-(6-methyl-2-oxo-1,2-dihydroquinolin-3-yl)-1-oxopropan-2-yl)-4-((S)-3,3-dimethyl-2-(2,2,2-trifluoroacetamido)butanoyl)hexahydro-2H-furo[3,2-b]pyrrole-5-carboxamide (P1.264)

To a solution of P1.263 (86.0 mg, 0.173 mol, 1.0 eq) in DCM (32 mL) was added triethylamine (48 μL, 0.346 mol, 2.0 eq) and 1 M trifluoroacetic anhydride in DCM (173 μL, 0.173 mol, 1.0 eq) at 0° C. After the reaction mixture was stirred at rt for 16 h, the solution was added by trimethylamine (2.0 eq) and 2 M trifluoroacetic anhydride in DCM (1 eq) again. After the starting material was consumed, the reaction was quenched with NaHCO_{3(aq)} and extracted with DCM. The organic layers were collected, dried over MgSO₄₍ₛ₎, filtered, and concentrated under reduced pressure. The residue was purified by C18 flash column chromatography (0-100% methanol in H₂O) to afford P1.264 (63 mg, 61%) as a white solid. LCMS (ESI) m/z calc. for C₂₈H₃₄F₃N₅O₆ 593.25. found, 594.0 [M+H]⁺.

### Synthesis of (1R,2S,5S)—N-(1-amino-3-(6,7-dimethyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.271)

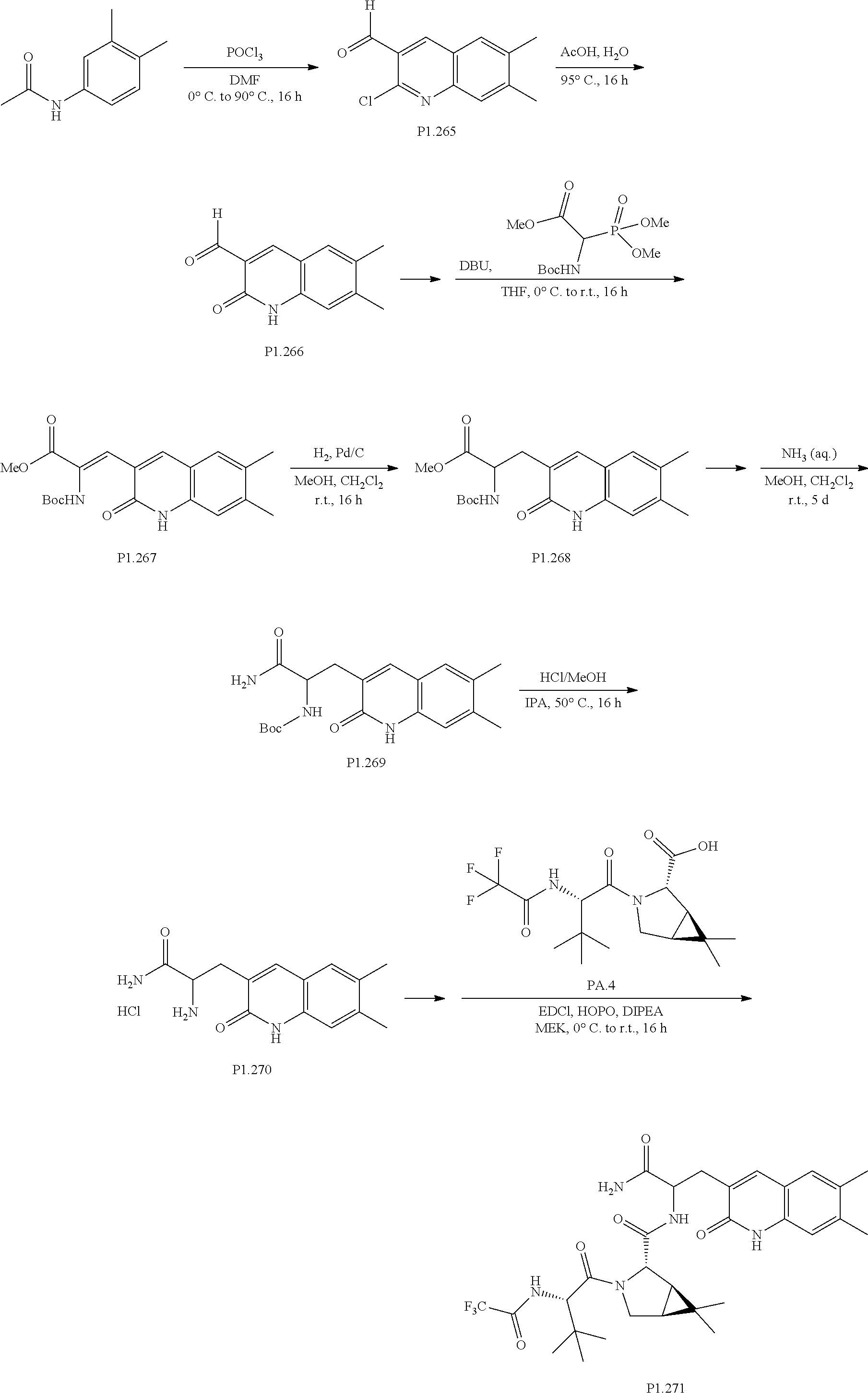

### Preparation 266. 2-Chloro-6,7-dimethylquinoline-3-carbaldehyde (P1.265)

A solution of phosphoryl chloride (52.0 mL, 551 mmol, 9 eq) in anh. DMF (13 mL) was stirred at 0° C. for 10 min, then warmed to rt and stirred for 15 min. To the mixture was added N-(3,4-dimethylphenyl)acetamide (10 g, 61.27 mmol, 1.0 eq) at 0° C. and then stirred at 90° C. for 16 h. The mixture was cooled, treated with ice water, and extracted with EtOAc. The combined organic layers were dried over Na₂SO₄, filtered, and concentrated to give P1.265 (11.1 g, 82% yield) as a yellow solid which was used in next step without further purification. ¹H NMR (400 MHz, CDCl₃), δ: 10.51 (s, 1H), 8.64 (s, 1H), 7.84 (s, 1H), 7.69 (s, 1H), 2.50 (s, 3H), 2.46 (s, 3H); LCMS (ESI) m/z calc. for C₁₂H₁₀ClNO 219.05. found, 220.1 [M+H]⁺.

### Preparation 267. 6,7-Dimethyl-2-oxo-1,2-dihydroquinoline-3-carbaldehyde (P1.266)

A solution of P1.265 (11.1 g, 50.5 mmol, 1.0 eq) in 70% AcOH_{(aq)} (505 mL) was stirred at 95° C. for 16 h. After the mixture was cooled to rt, the resulting precipitate was collected by filtration to give P1.266 (9.9 g, 97% yield) as a yellow solid. ¹H NMR (400 MHz, CDCl₃), δ: 11.77 (br. s, 1H), 10.49 (s, 1H), 8.41 (s, 1H), 7.45 (s, 1H), 7.19 (s, 1H), 2.41 (s, 3H), 2.33 (s, 3H); LCMS (ESI) m/z calc. for C₁₂H₁₁NO₂ 201.08. found, 202.1 [M+H]⁺.

### Preparation 268. Methyl (Z)-2-((tert-butoxycarbonyl)amino)-3-(6,7-dimethyl-2-oxo-1,2-dihydroquinolin-3-yl)acrylate (P1.267)

To a solution of (±)-boc-α-phosphonoglycine trimethyl ester (5.90 g, 19.7 mmol, 1.32 eq) in anh. THF (75 mL) was added 1,8-diazabicyclo[5.4.0]undec-7-ene (DBU, 3.0 mL, 20 mmol, 1.32 eq) slowly at 0° C. After the reaction was stirred at 0° C. for 30 min, the ylide solution was added slowly into a solution of P1.266 (3.0 g, 14.91 mmol, 1.0 eq) in anh. THF (75 mL) at 0° C. The mixture was stirred at room temperature for 16 h. The solvent was removed under reduced pressure. The residue was treated with methanol. The resulting precipitate was collected by filtration to give P1.267 (3.18 g, 57% yield) as a yellow solid. ¹H NMR (400 MHz, CDCl₃), δ: 8.75 (br. s, 1H), 7.87 (s, 1H), 7.31 (s, 1H), 7.13 (s, 1H), 6.83 (s, 1H), 3.87 (s, 3H), 2.40 (s, 3H), 2.32 (s, 3H), 1.41 (s, 9H); LCMS (ESI) m/z calc. for C₂₀H₂₄N₂O₅ 372.17. found, 373.2 [M+H]⁺.

### Preparation 269. Methyl 2-((tert-butoxycarbonyl)amino)-3-(6,7-dimethyl-2-oxo-1,2-dihydroquinolin-3-yl)propanoate (P1.268)

To a solution of P1.267 (3.18 g, 8.54 mmol, 1.0 eq) in methanol (50 mL) and DCM (150 mL) was added Pd/C (906 mg, 0.1 eq). The reaction was stirred at rt for 16 h under hydrogen (1 atm). The solution was filtered through a pad of celite. The filtrate was concentrated under reduced pressure to give P1.268 (3.0 g, 93% yield) as a yellow solid which was used in next step without further purification. ¹H NMR (400 MHz, CD₃OD), δ: 7.70 (s, 1H), 7.35 (s, 1H), 7.12 (s, 1H), 4.52-4.48 (m, 1H), 3.68 (s, 3H), 3.21-3.12 (m, 1H), 2.95-2.80 (m, 1H), 2.36 (s, 3H), 2.32 (s, 3H), 1.32 (s, 9H); LCMS (ESI) m/z calc. for C₂₀H₂₆N₂O₅ 374.18. found, 375.2 [M+H]⁺.

### Preparation 270. tert-Butyl (1-amino-3-(6,7-dimethyl-2-oxo-1,2-dihydroquinolin-3-yl)-1-oxopropan-2-yl)carbamate (P1.269)

To a solution of P1.268 (3.0 g, 8.0 mmol) in methanol (60 mL) and DCM (140 mL) was added 30% ammonium hydroxide solution (35 mL). The reaction was stirred at rt for 5 days. The solvent was removed under reduced pressure. The residue was treated with DCM. The resulting precipitate was collected by filtration and washed with dichloromethane give P1.269 (2.22 g, 77% yield) as a yellow solid which was used in next step without further purification. ¹H NMR (400 MHz, DMSO-d₆), δ: 11.67 (br., 1H), 7.58 (s, 1H), 7.29 (s, 1H), 7.21 (br. s, 1H), 7.04 (s, 1H), 6.96 (br. s, 1H), 6.92 (d, J=8.0 Hz, 1H), 4.16-4.07 (m, 1H), 2.91-2.86 (m, 1H), 2.66-2.60 (m, 1H), 2.26 (s, 3H), 2.23 (s, 3H), 1.25 (s, 9H); LCMS (ESI) m/z calc. for C₁₉H₂₅N₃O₄ 359.18. found, 360.2 [M+H]⁺.

### Preparation 271. 2-Amino-3-(6,7-dimethyl-2-oxo-1,2-dihydroquinolin-3-yl)propanamide hydrochloride (P1.270)

To a solution of P1.269 (2.22 g, 6.18 mmol, 1.0 eq) in isopropanol (41 mL) was added 3M HCl in MeOH (10.3 mL, 30.9 mmol, 5.0 eq). The reaction was stirred at 50° C. for 16 h. The resulting precipitate was collected by filtration, washed with methanol, and dried to give P1.270 (1.5 g, 82% yield) as a yellow solid. ¹H NMR (400 MHz, DMSO-d₆), δ: 11.88 (br. s, 1H), 8.25 (br. s, 3H), 7.92 (br. s, 1H), 7.71 (s, 1H), 7.51 (br. s, 1H), 7.36 (s, 1H), 7.11 (s, 1H), 4.03 (dd, J=7.6, 5.2 Hz, 1H), 3.01-2.89 (m, 2H), 2.28 (s, 3H), 2.25 (s, 3H); LCMS (ESI) m/z calc. for C₁₄H₁₇N₃O₂ 259.13. found, 260.1 [M+H]⁺.

### Preparation 272. (1R,2S,5S)—N-(1-amino-3-(6,7-dimethyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.271)

To a solution of P1.270 (250 mg, 0.85 mmol, 1.1 eq), PA.4 (280 mg, 0.77 mmol, 1.0 eq) in butan-2-one (2.8 mL) was added HOPO (21 mg, 0.19 mmol, 0.25 eq.) and EDCI (192 mg, 1.00 mmol, 1.3 eq.) at 0° C. After DIPEA (536 μL, 3.08 mmol, 4.0 eq) was added slowly to the reaction mixture, the resulting mixture was stirred at rt for 16 h. The mixture was concentrated under reduced pressure and then purified by C-18 reversed-phase column chromatography (0-100% MeOH in water containing 1% NH₄HCO₃) to give P1.271 (340 mg, 66% yield) as a yellow solid. ¹H NMR (400 MHz, DMSO-d₆), δ: 11.74-11.65 (m, 2H), 9.47-9.34 (m, 2H), 8.57-8.32 (m, 2H), 7.61-7.55 (m, 2H), 7.32-7.25 (m, 2H), 7.23-6.94 (m, 6H), 4.48-4.32 (m, 4H), 4.12-4.07 (m, 2H), 4.01-3.77 (m, 2H), 3.61-3.58 (m, 2H), 3.16-2.52 (m, 4H), 2.25-2.24 (m, 6H), 2.21-2.20 (m, 6H), 1.49-1.28 (m, 2H), 0.97-0.89 (m, 20H), 0.80-0.78 (m, 12H); LCMS (ESI) m/z calc. for C₃₀H₃₈F₃N₅O₅ 605.28. found, 606.4 [M+H]⁺.

### Synthesis of (1R,2S,5S)—N-(1-amino-3-(6,7-dimethyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P1.272)

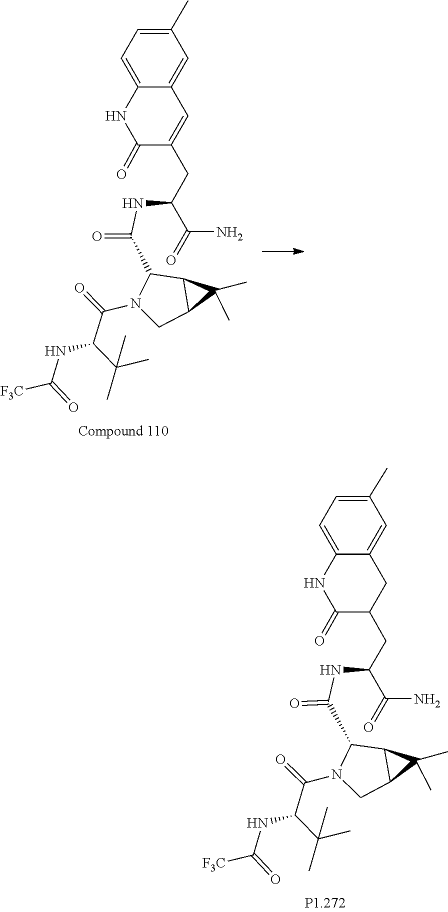

### Preparation 273. (1R,2S,5S)—N-[(1S)-1-cyano-2-(6-methyl-2-oxo-3,4-dihydro-1H-quinolin-3-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (P1.272)

To a solution of Compound 1 (900 mg, 1.5 mmol) in ethanol (50 mL) was added Pd/C (90 mg, 10% wt.). The reaction was stirred 48 h under hydrogen atmosphere (5 MPa) at 90° C. The solution was filtered through a pad of celite. The filtrate was concentrated under reduced pressure to give title compound (875 mg, 97%) as a solid. ¹H NMR (400 MHz, DMSO-d₆), δ: 10.05-9.91 (m, 1H), 9.26 (s, 1H), 8.22 (t, J=7.4 Hz, 1H), 7.36-7.19 (m, 1H), 7.06-6.97 (m, 2H), 6.92 (t, J=8.4 Hz, 1H), 6.71 (dd, J=11.2, 8.0 Hz, 1H), 4.43 (d, J=13.0 Hz, 1H), 4.32 (s, 2H), 4.27-4.13 (m, 1H), 3.94-3.81 (m, 1H), 3.65 (d, J=10.4 Hz, 1H), 3.44 (dd, J=7.2, 4.2 Hz, 2H), 3.05-2.91 (m, 1H), 2.70-2.54 (m, 1H), 2.20 (s, 3H), 1.63 (dd, J=15.5, 10.0 Hz, 1H), 1.48 (q, J=6.8 Hz, 1H), 1.36 (dd, J=7.7, 4.4 Hz, 1H), 1.10-0.93 (m, 15H), 0.87 (d, J=13.8 Hz, 1H), 0.80 (q, J=7.7 Hz, 7H).

### Synthesis of (1R,2S,5S)—N-[2-amino-1-[(6-methyl-2-oxo-3,4-dihydro-1H-quinolin-3-yl)methyl]-2-oxo-ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (P1.277)

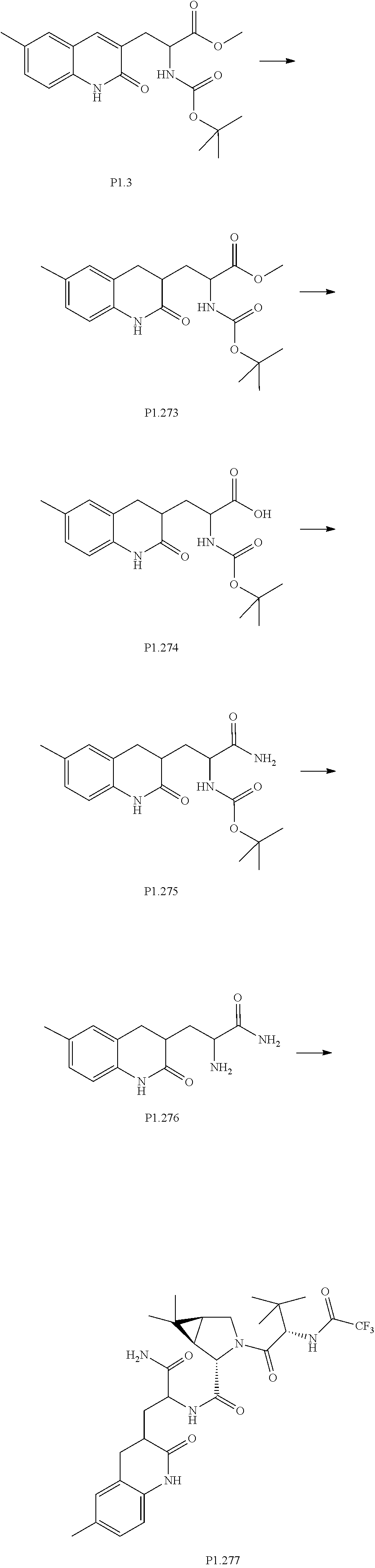

### Preparation 274. Methyl 2-(tert-butoxycarbonylamino)-3-(6-methyl-2-oxo-3,4-dihydro-1H-quinolin-3-yl)propanoate (P1.273)

To a solution of P1.3 (200 mg, 0.55 mmol) in ethanol (50 mL) was added Pd/C (20 mg, 10% wt.). The reaction was stirred for 48 h under hydrogen atmosphere (5 MPa) at 90° C. The solution was filtered through a pad of celite. The filtrate was concentrated under reduced pressure to give P1.273 (200 mg, 98%) as a solid. ¹H NMR (400 MHz, DMSO-d₆), δ: 10.05 (d, J=2.7 Hz, 1H), 7.38-7.25 (m, 1H), 6.94 (d, J=10.4 Hz, 2H), 6.73 (d, J=7.8 Hz, 1H), 4.26 (s, 1H), 4.15 (s, 1H), 3.62 (d, J=3.5 Hz, 3H), 2.86 (dd, J=15.6, 5.9 Hz, 1H), 2.63 (t, J=13.7 Hz, 1H), 2.42 (s, 1H), 2.21 (s, 3H), 2.13 (s, 1H), 1.60-1.51 (m, OH), 1.36 (s, 10H).

### Preparation 275. 2-(tert-Butoxycarbonylamino)-3-(6-methyl-2-oxo-3,4-dihydro-1H-quinolin-3-yl)propanoic acid (P1.274)

A mixture of P1.273 (200 mg, 0.55 mmol), NaOH (33 mg, 0.83 mmol) in 5 mL MeOH and 1 mL H₂O was stirred at 60° C. for 14 h and evaporated. The residue was diluted with water and acidified with 6M HCl to pH=3-4. The formed solid was filtered off and dried. Yield of P1.274: 190 mg, 99%. ¹H NMR (400 MHz, DMSO-d₆), δ: 12.49 (s, 2H), 10.03 (s, 2H), 7.17 (d, J=8.4 Hz, 1H), 7.11 (d, J=7.8 Hz, 1H), 6.94 (d, J=10.5 Hz, 4H), 6.73 (d, J=7.9 Hz, 2H), 4.13 (s, 1H), 4.04 (s, 1H), 2.87 (d, J=16.1 Hz, 2H), 2.63 (t, J=13.5 Hz, 2H), 2.53-2.48 (m, 7H), 2.42 (s, 3H), 2.21 (s, 5H), 2.12 (d, J=12.3 Hz, 3H), 1.68 (s, 2H), 1.54 (s, 1H), 1.36 (s, 18H), 0.98 (d, J=6.4 Hz, 1H).

### Preparation 276. tert-Butyl N-[2-amino-1-[(6-methyl-2-oxo-3,4-dihydro-1H-quinolin-3-yl)methyl]-2-oxo-ethyl]carbamate (P1.275)

To a mixture of the P1.274 (190 mg, 0.55 mmol) and HATU (239 mg, 0.6 mmol) in DMF (2 ml) DIPEA (96 mg, 0.71 mmol) was added. The mixture was stirred at rt for 30 min, and ammonium carbonate (110 mg, 1.1 mmol) was added, and stirred overnight. The reaction mixture was diluted with ethyl acetate (50 ml) and washed with water (3×50 ml), brine (2×50 mL), dried over Na₂SO₄, and concentrated under reduced pressure. Yield of P1.275: 190 mg, 95%.

### Preparation 277. 2-Amino-3-(6-methyl-2-oxo-3,4-dihydro-1H-quinolin-3-yl)propanamide (P1.276)

To a solution of the P1.275 (190 mg, 0.54 mmol) in dioxane (5 mL) HCl (10 mL, 3M in dioxane) was added. The reaction was stirred at 50° C. for overnight. The solvent was removed under reduced pressure to give P1.276 (150 mg, 95%).

### Preparation 278. (1R,2S,5S)—N-[2-Amino-1-[(6-methyl-2-oxo-3,4-dihydro-1H-quinolin-3-yl)methyl]-2-oxo-ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (P1.277)

To a mixture of the PA.4 (202 mg, 0.55 mmol) and HATU (221 mg, 0.58 mmol) in DMF (2 ml) DIPEA (150 mg, 1.17 mmol) was added. The mixture was stirred at rt for 30 min, and P1.276 (150 mg, 0.53 mmol) was added, and stirred overnight. Reaction mixture was diluted with ethyl acetate (50 ml) and washed with water (3×50 ml), brine (2×50 mL), dried on Na₂SO₄, and concentrated under reduced pressure to give P1.277 (245 mg, 780%).

### Examples of the Final Compound

In the Table 4 presented certain non-limiting examples of the compound of Formula (A).

TABLE 4
Selected examples of the compound of Formula (A)

| | | [MH]⁺ | [MH]⁺ |
|---|---|---|---|
| # | Structure | Calc. | Found |
| 101 | 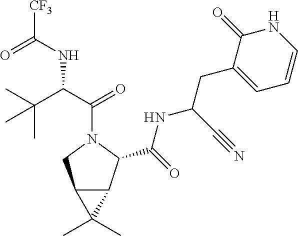 [SMILES: CC(C)(C)[C@H](NC(=O)C(F)(F)F)C(=O)N1C[C@H]2[C@H]([C@H]1C(=O)NC(C#N)Cc1ccc[nH]c1=O)C2(C)C] | 510.2328 | 510 |
| 102 | 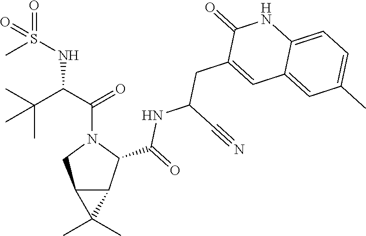 [SMILES: Cc1ccc2[nH]c(=O)c(CC(C#N)NC(=O)[C@@H]3[C@H]4[C@H](CN3C(=O)[C@@H](NS(C)(=O)=O)C(C)(C)C)C4(C)C)cc2c1] | 556.2593 | 556 |
| 103 | 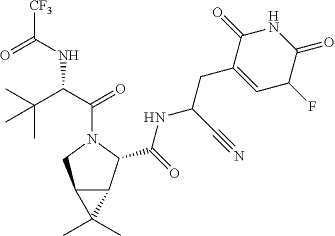 [SMILES: CC(C)(C)[C@H](NC(=O)C(F)(F)F)C(=O)N1C[C@H]2[C@H]([C@H]1C(=O)NC(C#N)CC1=CC(F)C(=O)NC1=O)C2(C)C] | 544.2184 | 544 |
| 104 | 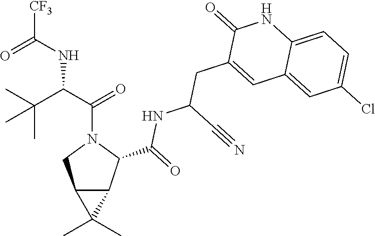 [SMILES: CC(C)(C)[C@H](NC(=O)C(F)(F)F)C(=O)N1C[C@H]2[C@H]([C@H]1C(=O)NC(C#N)Cc1cc3cc(Cl)ccc3[nH]c1=O)C2(C)C] | 594.2095 | 594 |
| 105 | 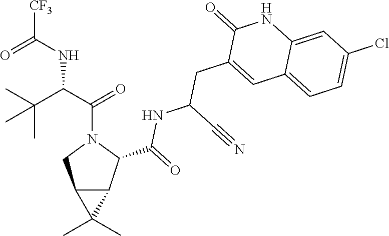 [SMILES: CC(C)(C)[C@H](NC(=O)C(F)(F)F)C(=O)N1C[C@H]2[C@H]([C@H]1C(=O)NC(C#N)Cc1cc3ccc(Cl)cc3[nH]c1=O)C2(C)C] | 594.2095 | 594 |
| 106 | 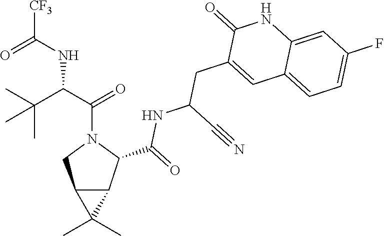 [SMILES: CC(C)(C)[C@H](NC(=O)C(F)(F)F)C(=O)N1C[C@H]2[C@H]([C@H]1C(=O)NC(C#N)Cc1cc3ccc(F)cc3[nH]c1=O)C2(C)C] | 578.239 | 578 |
| 107 | 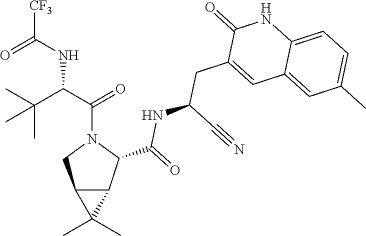 [SMILES: Cc1ccc2[nH]c(=O)c(C[C@@H](C#N)NC(=O)[C@@H]3[C@H]4[C@H](CN3C(=O)[C@@H](NC(=O)C(F)(F)F)C(C)(C)C)C4(C)C)cc2c1] | 574.2641 | 574 |
| 108 | 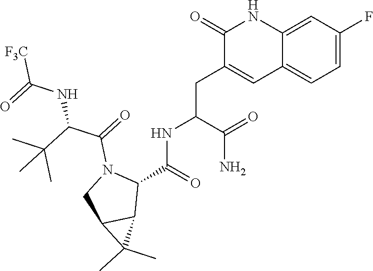 [SMILES: CC(C)(C)[C@H](NC(=O)C(F)(F)F)C(=O)N1C[C@H]2[C@H]([C@H]1C(=O)NC(Cc1cc3ccc(F)cc3[nH]c1=O)C(N)=O)C2(C)C] | 596.2496 | 596 |
| 109 | 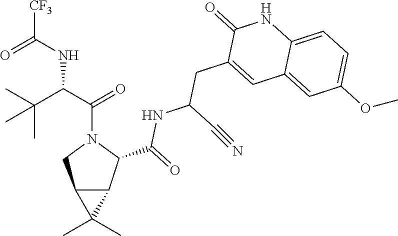 [SMILES: COc1ccc2[nH]c(=O)c(CC(C#N)NC(=O)[C@@H]3[C@H]4[C@H](CN3C(=O)[C@@H](NC(=O)C(F)(F)F)C(C)(C)C)C4(C)C)cc2c1] | 590.2591 | 590 |
| 110 | 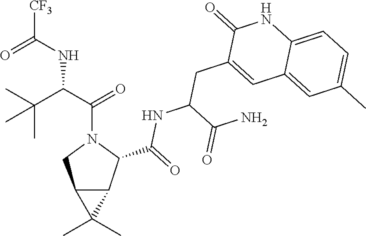 [SMILES: Cc1ccc2[nH]c(=O)c(CC(NC(=O)[C@@H]3[C@H]4[C@H](CN3C(=O)[C@@H](NC(=O)C(F)(F)F)C(C)(C)C)C4(C)C)C(N)=O)cc2c1] | 592.2747 | 592 |
| 111 | 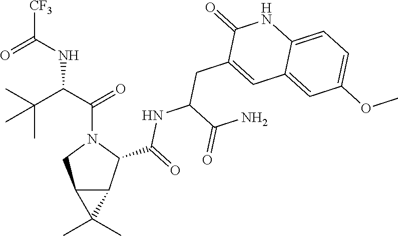 [SMILES: COc1ccc2[nH]c(=O)c(CC(NC(=O)[C@@H]3[C@H]4[C@H](CN3C(=O)[C@@H](NC(=O)C(F)(F)F)C(C)(C)C)C4(C)C)C(N)=O)cc2c1] | 608.2696 | 608 |
| 112 | 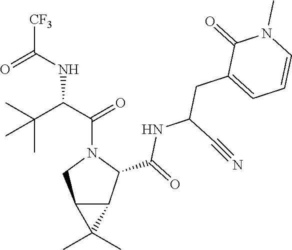 [SMILES: Cn1cccc(CC(C#N)NC(=O)[C@@H]2[C@H]3[C@H](CN2C(=O)[C@@H](NC(=O)C(F)(F)F)C(C)(C)C)C3(C)C)c1=O] | 524.2485 | 524 |
| 113 | 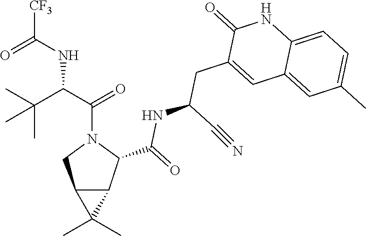 [SMILES: Cc1ccc2[nH]c(=O)c(C[C@@H](C#N)NC(=O)[C@@H]3[C@H]4[C@H](CN3C(=O)[C@@H](NC(=O)C(F)(F)F)C(C)(C)C)C4(C)C)cc2c1] | 574.2641 | 574 |
| 114 | 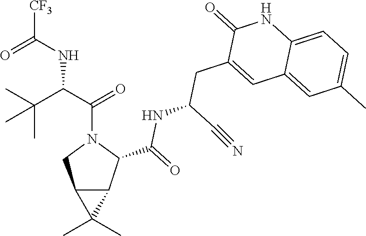 [SMILES: Cc1ccc2[nH]c(=O)c(C[C@H](C#N)NC(=O)[C@@H]3[C@H]4[C@H](CN3C(=O)[C@@H](NC(=O)C(F)(F)F)C(C)(C)C)C4(C)C)cc2c1] | 574.2641 | 574 |
| 115 | 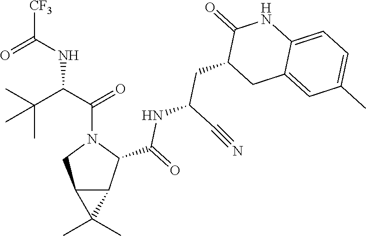 [SMILES: Cc1ccc2c(c1)C[C@H](C[C@H](C#N)NC(=O)[C@@H]1[C@H]3[C@H](CN1C(=O)[C@@H](NC(=O)C(F)(F)F)C(C)(C)C)C3(C)C)C(=O)N2] | 576.2797 | 576 |
| 116 | 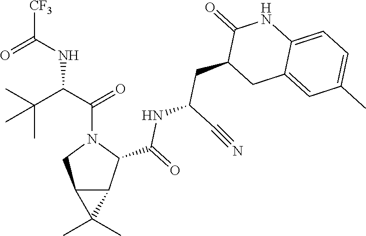 [SMILES: Cc1ccc2c(c1)C[C@@H](C[C@H](C#N)NC(=O)[C@@H]1[C@H]3[C@H](CN1C(=O)[C@@H](NC(=O)C(F)(F)F)C(C)(C)C)C3(C)C)C(=O)N2] | 576.2797 | 576 |
| 117 | 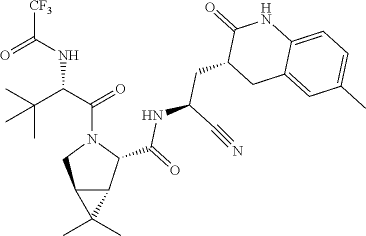 [SMILES: Cc1ccc2c(c1)C[C@H](C[C@@H](C#N)NC(=O)[C@@H]1[C@H]3[C@H](CN1C(=O)[C@@H](NC(=O)C(F)(F)F)C(C)(C)C)C3(C)C)C(=O)N2] | 576.2797 | 576 |
| 118 | 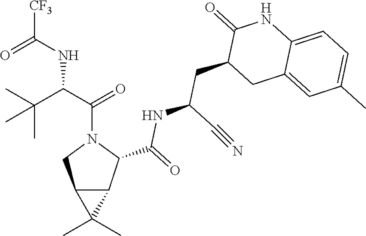 [SMILES: Cc1ccc2c(c1)C[C@@H](C[C@@H](C#N)NC(=O)[C@@H]1[C@H]3[C@H](CN1C(=O)[C@@H](NC(=O)C(F)(F)F)C(C)(C)C)C3(C)C)C(=O)N2] | 576.2797 | 576 |
| 119 | 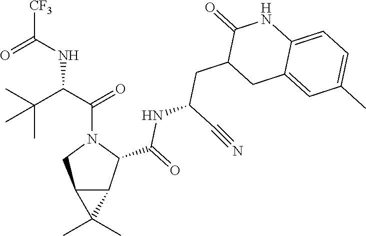 [SMILES: Cc1ccc2c(c1)CC(C[C@H](C#N)NC(=O)[C@@H]1[C@H]3[C@H](CN1C(=O)[C@@H](NC(=O)C(F)(F)F)C(C)(C)C)C3(C)C)C(=O)N2] | 576.2797 | 576 |
| 120 | 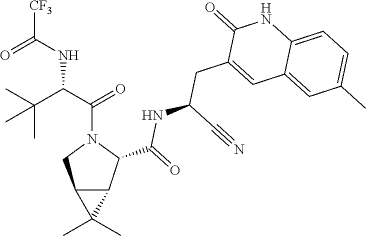 [SMILES: Cc1ccc2[nH]c(=O)c(C[C@@H](C#N)NC(=O)[C@@H]3[C@H]4[C@H](CN3C(=O)[C@@H](NC(=O)C(F)(F)F)C(C)(C)C)C4(C)C)cc2c1] | 576.2797 | 576 |
| 121 | 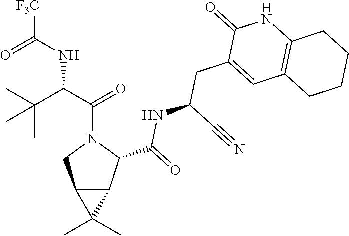 [SMILES: CC(C)(C)[C@H](NC(=O)C(F)(F)F)C(=O)N1C[C@H]2[C@H]([C@H]1C(=O)N[C@H](C#N)Cc1cc3c([nH]c1=O)CCCC3)C2(C)C] | 564.2797 | 564 |
| 122 | 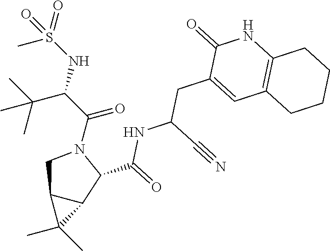 [SMILES: CC(C)(C)[C@H](NS(C)(=O)=O)C(=O)N1C[C@H]2[C@H]([C@H]1C(=O)NC(C#N)Cc1cc3c([nH]c1=O)CCCC3)C2(C)C] | 546.275 | 546 |
| 123 | 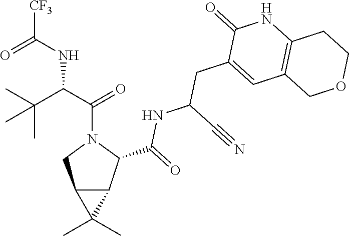 [SMILES: CC(C)(C)[C@H](NC(=O)C(F)(F)F)C(=O)N1C[C@H]2[C@H]([C@H]1C(=O)NC(C#N)Cc1cc3c([nH]c1=O)CCOC3)C2(C)C] | 566.259 | 566 |
| 124 | 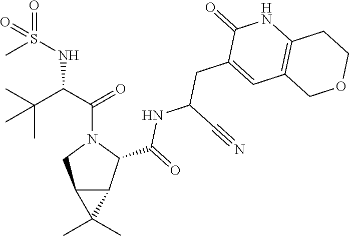 [SMILES: CC(C)(C)[C@H](NS(C)(=O)=O)C(=O)N1C[C@H]2[C@H]([C@H]1C(=O)NC(C#N)Cc1cc3c([nH]c1=O)CCOC3)C2(C)C] | 548.2543 | 548 |
| 125 | 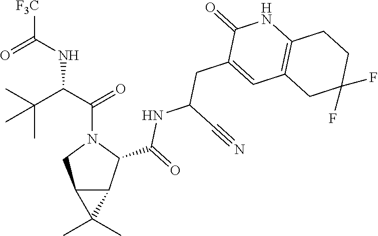 [SMILES: CC(C)(C)[C@H](NC(=O)C(F)(F)F)C(=O)N1C[C@H]2[C@H]([C@H]1C(=O)NC(C#N)Cc1cc3c([nH]c1=O)CCC(F)(F)C3)C2(C)C] | 600.2609 | 600 |
| 126 | 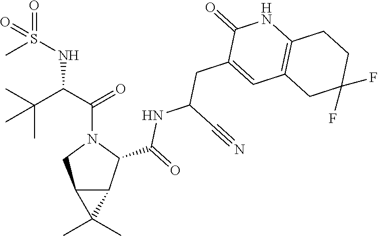 [SMILES: CC(C)(C)[C@H](NS(C)(=O)=O)C(=O)N1C[C@H]2[C@H]([C@H]1C(=O)NC(C#N)Cc1cc3c([nH]c1=O)CCC(F)(F)C3)C2(C)C] | 582.2562 | 582 |
| 127 | 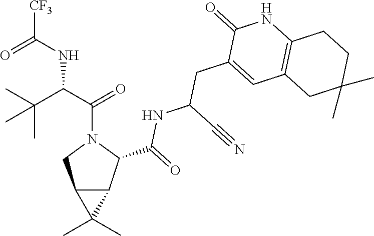 [SMILES: CC1(C)CCc2[nH]c(=O)c(CC(C#N)NC(=O)[C@@H]3[C@H]4[C@H](CN3C(=O)[C@@H](NC(=O)C(F)(F)F)C(C)(C)C)C4(C)C)cc2C1] | 592.311 | 592 |
| 128 | 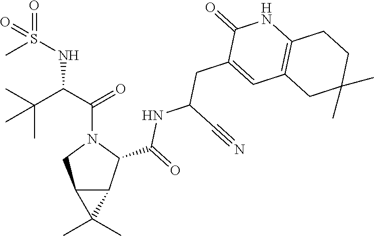 [SMILES: CC1(C)CCc2[nH]c(=O)c(CC(C#N)NC(=O)[C@@H]3[C@H]4[C@H](CN3C(=O)[C@@H](NS(C)(=O)=O)C(C)(C)C)C4(C)C)cc2C1] | 574.3063 | 574 |
| 129 | 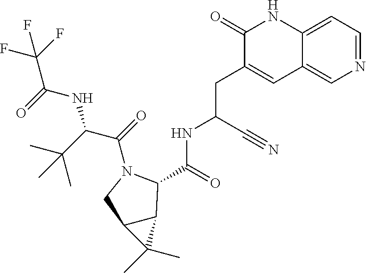 [SMILES: CC(C)(C)[C@H](NC(=O)C(F)(F)F)C(=O)N1C[C@H]2[C@H]([C@H]1C(=O)NC(C#N)Cc1cc3cnccc3[nH]c1=O)C2(C)C] | 561.2437 | 561 |
| 130 | 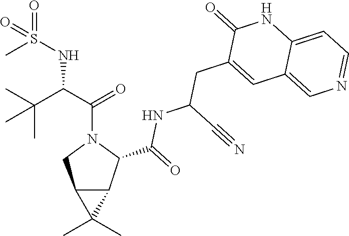 [SMILES: CC(C)(C)[C@H](NS(C)(=O)=O)C(=O)N1C[C@H]2[C@H]([C@H]1C(=O)NC(C#N)Cc1cc3cnccc3[nH]c1=O)C2(C)C] | 543.2389 | 543 |
| 131 | 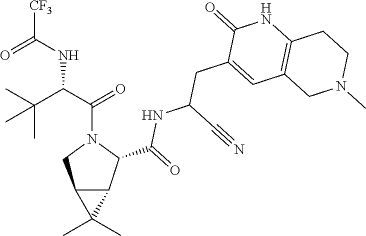 [SMILES: CN1CCc2[nH]c(=O)c(CC(C#N)NC(=O)[C@@H]3[C@H]4[C@H](CN3C(=O)[C@@H](NC(=O)C(F)(F)F)C(C)(C)C)C4(C)C)cc2C1] | 579.2906 | 579 |
| 132 | 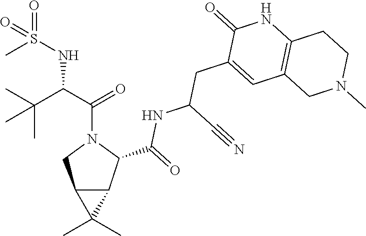 [SMILES: CN1CCc2[nH]c(=O)c(CC(C#N)NC(=O)[C@@H]3[C@H]4[C@H](CN3C(=O)[C@@H](NS(C)(=O)=O)C(C)(C)C)C4(C)C)cc2C1] | 561.2859 | 561 |
| 133 | 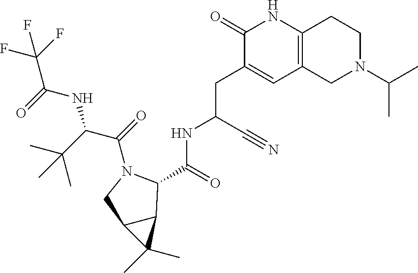 [SMILES: CC(C)N1CCc2[nH]c(=O)c(CC(C#N)NC(=O)[C@@H]3[C@@H]4[C@H](CN3C(=O)[C@@H](NC(=O)C(F)(F)F)C(C)(C)C)C4(C)C)cc2C1] | 607.3219 | 607 |
| 134 | 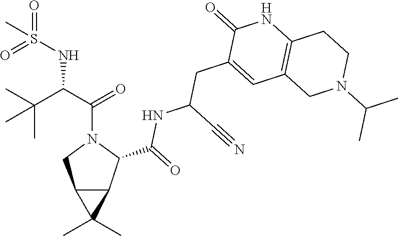 [SMILES: CC(C)N1CCc2[nH]c(=O)c(CC(C#N)NC(=O)[C@@H]3[C@@H]4[C@H](CN3C(=O)[C@@H](NS(C)(=O)=O)C(C)(C)C)C4(C)C)cc2C1] | 589.3172 | 589 |
| 135 | 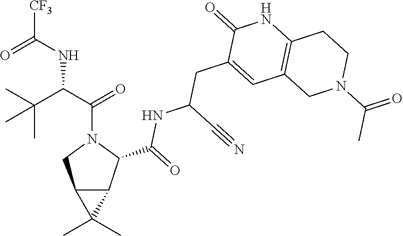 [SMILES: CC(=O)N1CCc2[nH]c(=O)c(CC(C#N)NC(=O)[C@@H]3[C@H]4[C@H](CN3C(=O)[C@@H](NC(=O)C(F)(F)F)C(C)(C)C)C4(C)C)cc2C1] | 607.2856 | 607 |
| 136 | 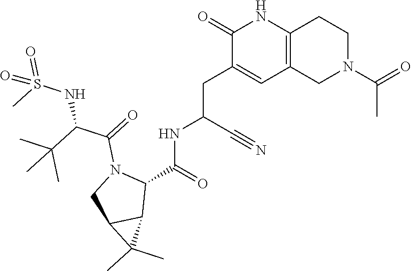 [SMILES: CC(=O)N1CCc2[nH]c(=O)c(CC(C#N)NC(=O)[C@@H]3[C@H]4[C@H](CN3C(=O)[C@@H](NS(C)(=O)=O)C(C)(C)C)C4(C)C)cc2C1] | 589.2808 | 589 |
| 137 | 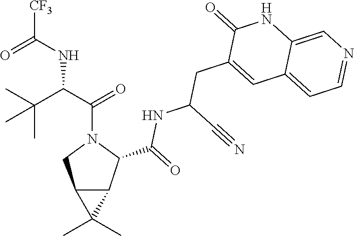 [SMILES: CC(C)(C)[C@H](NC(=O)C(F)(F)F)C(=O)N1C[C@H]2[C@H]([C@H]1C(=O)NC(C#N)Cc1cc3ccncc3[nH]c1=O)C2(C)C] | 561.2437 | 561 |
| 138 | 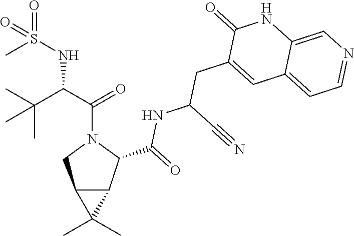 [SMILES: CC(C)(C)[C@H](NS(C)(=O)=O)C(=O)N1C[C@H]2[C@H]([C@H]1C(=O)NC(C#N)Cc1cc3ccncc3[nH]c1=O)C2(C)C] | 543.2389 | 543 |
| 138 | 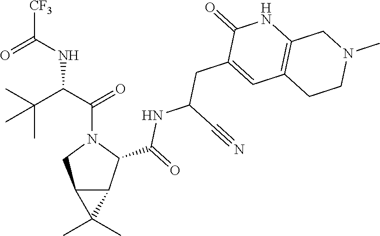 [SMILES: CN1CCc2cc(CC(C#N)NC(=O)[C@@H]3[C@H]4[C@H](CN3C(=O)[C@@H](NC(=O)C(F)(F)F)C(C)(C)C)C4(C)C)c(=O)[nH]c2C1] | 579.2906 | 579 |
| 140 | 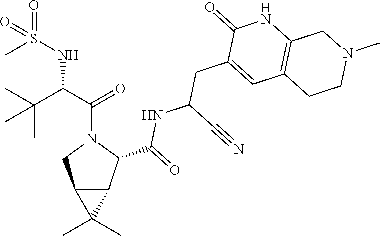 [SMILES: CN1CCc2cc(CC(C#N)NC(=O)[C@@H]3[C@H]4[C@H](CN3C(=O)[C@@H](NS(C)(=O)=O)C(C)(C)C)C4(C)C)c(=O)[nH]c2C1] | 561.2859 | 561 |
| 141 | 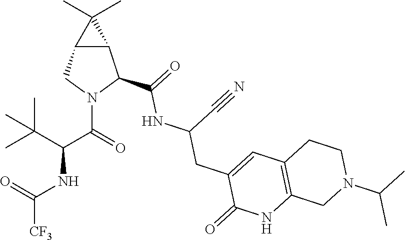 [SMILES: CC(C)N1CCc2cc(CC(C#N)NC(=O)[C@@H]3[C@@H]4[C@H](CN3C(=O)[C@@H](NC(=O)C(F)(F)F)C(C)(C)C)C4(C)C)c(=O)[nH]c2C1] | 607.3219 | 607 |
| 142 | 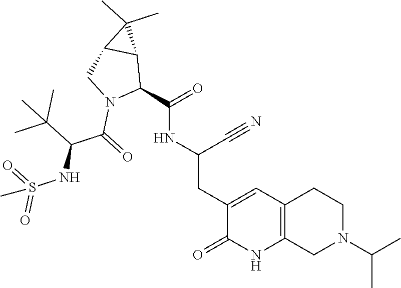 [SMILES: CC(C)N1CCc2cc(CC(C#N)NC(=O)[C@@H]3[C@@H]4[C@H](CN3C(=O)[C@@H](NS(C)(=O)=O)C(C)(C)C)C4(C)C)c(=O)[nH]c2C1] | 589.3172 | 589 |
| 143 | 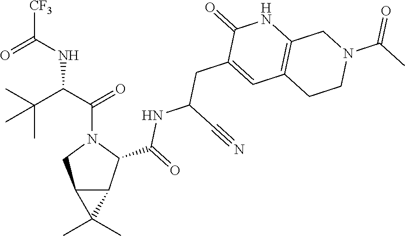 [SMILES: CC(=O)N1CCc2cc(CC(C#N)NC(=O)[C@@H]3[C@H]4[C@H](CN3C(=O)[C@@H](NC(=O)C(F)(F)F)C(C)(C)C)C4(C)C)c(=O)[nH]c2C1] | 607.2856 | 607 |
| 144 | 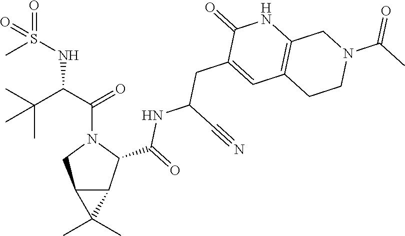 [SMILES: CC(=O)N1CCc2cc(CC(C#N)NC(=O)[C@@H]3[C@H]4[C@H](CN3C(=O)[C@@H](NS(C)(=O)=O)C(C)(C)C)C4(C)C)c(=O)[nH]c2C1] | 589.2808 | 589 |
| 145 | 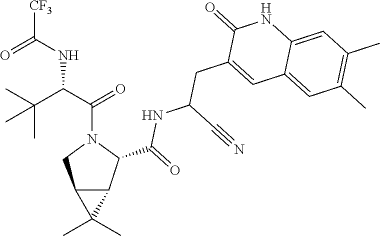 [SMILES: Cc1cc2cc(CC(C#N)NC(=O)[C@@H]3[C@H]4[C@H](CN3C(=O)[C@@H](NC(=O)C(F)(F)F)C(C)(C)C)C4(C)C)c(=O)[nH]c2cc1C] | 588.2797 | 588 |
| 146 | 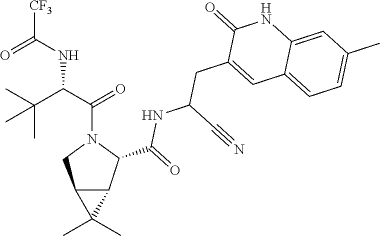 [SMILES: Cc1ccc2cc(CC(C#N)NC(=O)[C@@H]3[C@H]4[C@H](CN3C(=O)[C@@H](NC(=O)C(F)(F)F)C(C)(C)C)C4(C)C)c(=O)[nH]c2c1] | 574.2641 | 574 |
| 147 | 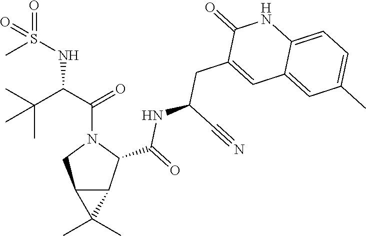 [SMILES: Cc1ccc2[nH]c(=O)c(C[C@@H](C#N)NC(=O)[C@@H]3[C@H]4[C@H](CN3C(=O)[C@@H](NS(C)(=O)=O)C(C)(C)C)C4(C)C)cc2c1] | 556.2593 | 556 |
| 148 | 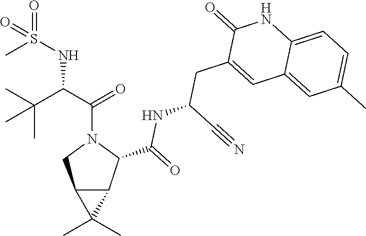 [SMILES: Cc1ccc2[nH]c(=O)c(C[C@H](C#N)NC(=O)[C@@H]3[C@H]4[C@H](CN3C(=O)[C@@H](NS(C)(=O)=O)C(C)(C)C)C4(C)C)cc2c1] | 556.2593 | 556 |
| 149 | 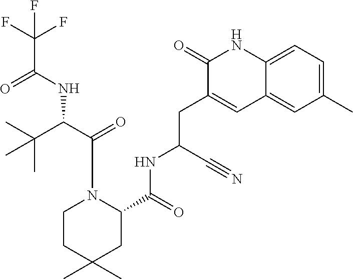 [SMILES: Cc1ccc2[nH]c(=O)c(CC(C#N)NC(=O)[C@@H]3CC(C)(C)CCN3C(=O)[C@@H](NC(=O)C(F)(F)F)C(C)(C)C)cc2c1] | 576.2797 | 576 |
| 150 | 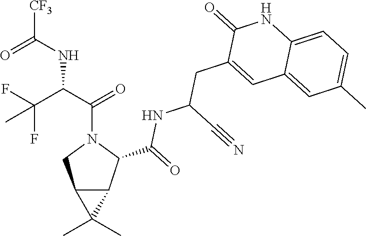 [SMILES: Cc1ccc2[nH]c(=O)c(CC(C#N)NC(=O)[C@@H]3[C@H]4[C@H](CN3C(=O)[C@@H](NC(=O)C(F)(F)F)C(C)(F)F)C4(C)C)cc2c1] | 582.2139 | 582 |
| 151 | 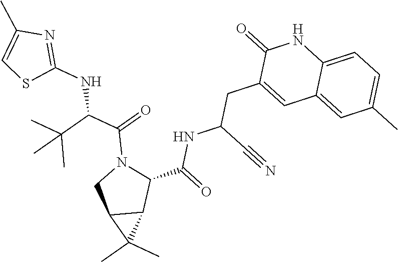 | 575.2804 | 575 |
| 152 | 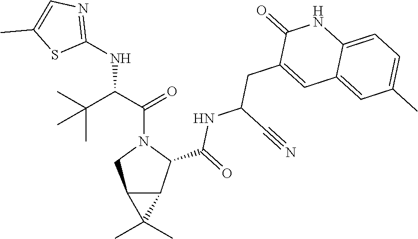 | 575.2804 | 575 |
| 153 | 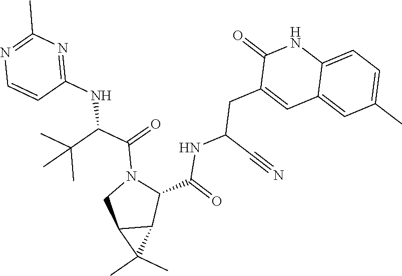 [SMILES: Cc1ccc2[nH]c(=O)c(CC(C#N)NC(=O)[C@@H]3[C@H]4[C@H](CN3C(=O)[C@@H](Nc3ccnc(C)n3)C(C)(C)C)C4(C)C)cc2c1] | 570.3193 | 570 |
| 154 | 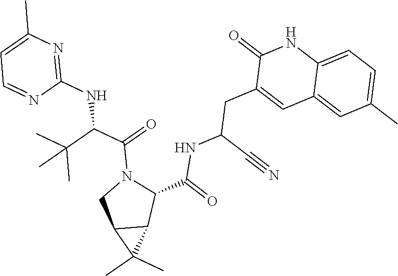 [SMILES: Cc1ccc2[nH]c(=O)c(CC(C#N)NC(=O)[C@@H]3[C@H]4[C@H](CN3C(=O)[C@@H](Nc3nccc(C)n3)C(C)(C)C)C4(C)C)cc2c1] | 570.3193 | 570 |
| 155 | 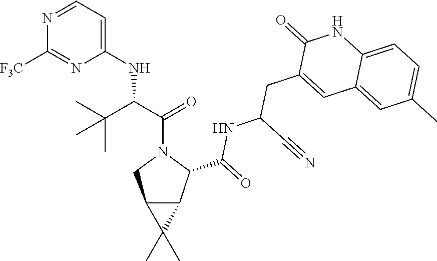 [SMILES: Cc1ccc2[nH]c(=O)c(CC(C#N)NC(=O)[C@@H]3[C@H]4[C@H](CN3C(=O)[C@@H](Nc3ccnc(C(F)(F)F)n3)C(C)(C)C)C4(C)C)cc2c1] | 624.2909 | 624 |
| 156 | 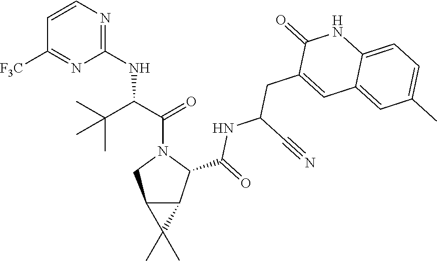 [SMILES: Cc1ccc2[nH]c(=O)c(CC(C#N)NC(=O)[C@@H]3[C@H]4[C@H](CN3C(=O)[C@@H](Nc3nccc(C(F)(F)F)n3)C(C)(C)C)C4(C)C)cc2c1] | 624.2909 | 624 |
| 157 | 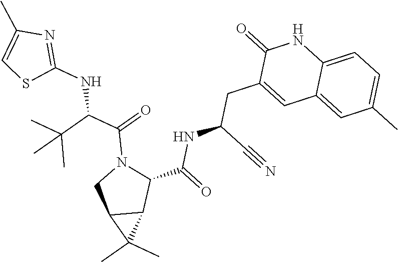 | 575.2804 | 575 |
| 158 | 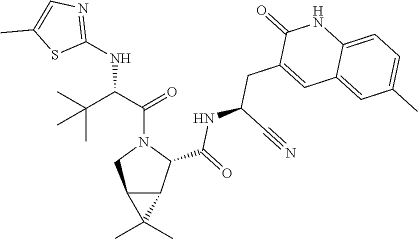 | 575.2804 | 575 |
| 159 | 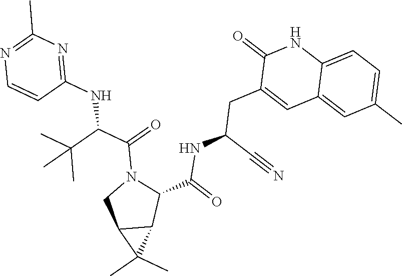 [SMILES: Cc1ccc2[nH]c(=O)c(C[C@@H](C#N)NC(=O)[C@@H]3[C@H]4[C@H](CN3C(=O)[C@@H](Nc3ccnc(C)n3)C(C)(C)C)C4(C)C)cc2c1] | 570.3193 | 570 |
| 160 | 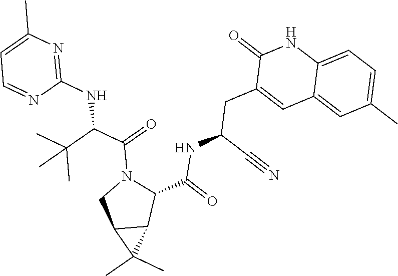 [SMILES: Cc1ccc2[nH]c(=O)c(C[C@@H](C#N)NC(=O)[C@@H]3[C@H]4[C@H](CN3C(=O)[C@@H](Nc3nccc(C)n3)C(C)(C)C)C4(C)C)cc2c1] | 570.3193 | 570 |
| 161 | 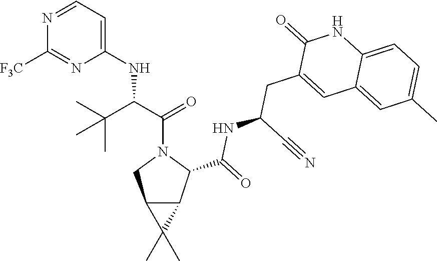 [SMILES: Cc1ccc2[nH]c(=O)c(C[C@@H](C#N)NC(=O)[C@@H]3[C@H]4[C@H](CN3C(=O)[C@@H](Nc3ccnc(C(F)(F)F)n3)C(C)(C)C)C4(C)C)cc2c1] | 624.2909 | 624 |
| 162 | 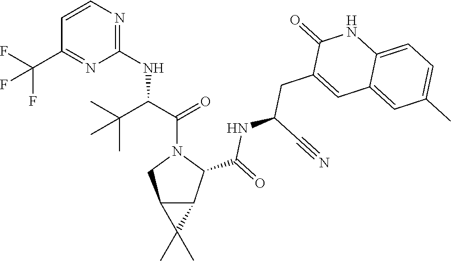 [SMILES: Cc1ccc2[nH]c(=O)c(C[C@@H](C#N)NC(=O)[C@@H]3[C@H]4[C@H](CN3C(=O)[C@@H](Nc3nccc(C(F)(F)F)n3)C(C)(C)C)C4(C)C)cc2c1] | 624.2909 | 624 |
| 163 | 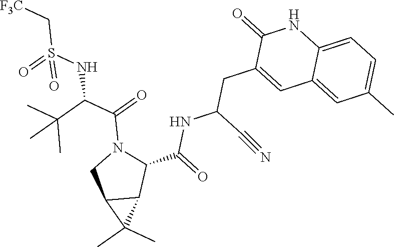 [SMILES: Cc1ccc2[nH]c(=O)c(CC(C#N)NC(=O)[C@@H]3[C@H]4[C@H](CN3C(=O)[C@@H](NS(=O)(=O)CC(F)(F)F)C(C)(C)C)C4(C)C)cc2c1] | 624.2468 | 624 |
| 164 | 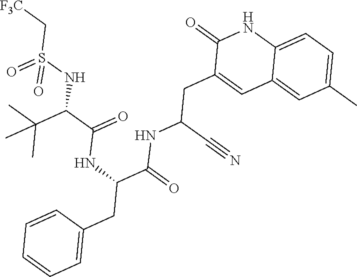 [SMILES: Cc1ccc2[nH]c(=O)c(CC(C#N)NC(=O)[C@H](Cc3ccccc3)NC(=O)[C@@H](NS(=O)(=O)CC(F)(F)F)C(C)(C)C)cc2c1] | 634.2311 | 634 |
| 165 | 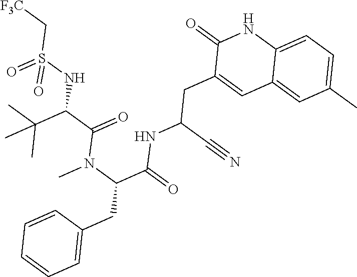 [SMILES: Cc1ccc2[nH]c(=O)c(CC(C#N)NC(=O)[C@H](Cc3ccccc3)N(C)C(=O)[C@@H](NS(=O)(=O)CC(F)(F)F)C(C)(C)C)cc2c1] | 648.2468 | 648 |
| 166 | 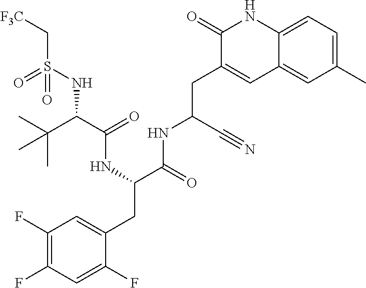 [SMILES: Cc1ccc2[nH]c(=O)c(CC(C#N)NC(=O)[C@H](Cc3cc(F)c(F)cc3F)NC(=O)[C@@H](NS(=O)(=O)CC(F)(F)F)C(C)(C)C)cc2c1] | 688.2028 | 688 |
| 167 | 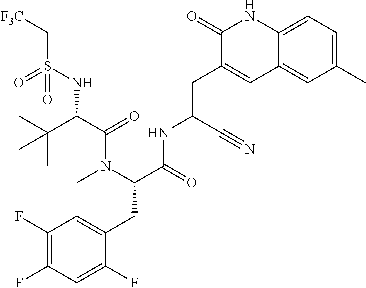 [SMILES: Cc1ccc2[nH]c(=O)c(CC(C#N)NC(=O)[C@H](Cc3cc(F)c(F)cc3F)N(C)C(=O)[C@@H](NS(=O)(=O)CC(F)(F)F)C(C)(C)C)cc2c1] | 702.2184 | 702 |
| 168 | 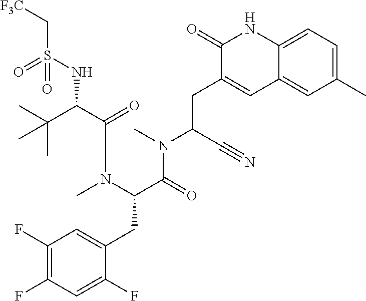 [SMILES: Cc1ccc2[nH]c(=O)c(CC(C#N)N(C)C(=O)[C@H](Cc3cc(F)c(F)cc3F)N(C)C(=O)[C@@H](NS(=O)(=O)CC(F)(F)F)C(C)(C)C)cc2c1] | 716.2341 | 716 |
| 169 | 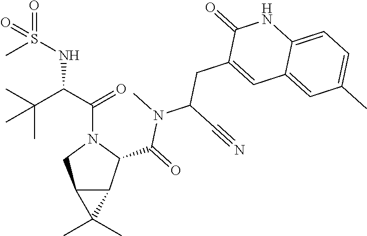 [SMILES: Cc1ccc2[nH]c(=O)c(CC(C#N)N(C)C(=O)[C@@H]3[C@H]4[C@H](CN3C(=O)[C@@H](NS(C)(=O)=O)C(C)(C)C)C4(C)C)cc2c1] | 570.275 | 570 |
| 170 | 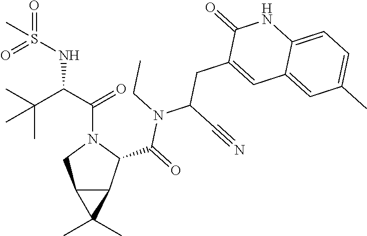 [SMILES: CCN(C(=O)[C@@H]1[C@@H]2[C@H](CN1C(=O)[C@@H](NS(C)(=O)=O)C(C)(C)C)C2(C)C)C(C#N)Cc1cc2cc(C)ccc2[nH]c1=O] | 584.2906 | 584 |
| 171 | 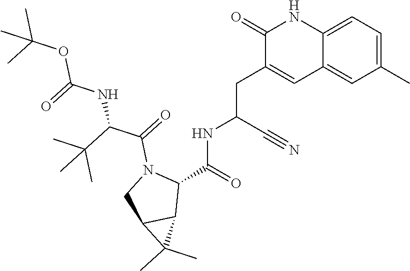 [SMILES: Cc1ccc2[nH]c(=O)c(CC(C#N)NC(=O)[C@@H]3[C@H]4[C@H](CN3C(=O)[C@@H](NC(=O)OC(C)(C)C)C(C)(C)C)C4(C)C)cc2c1] | 578.3342 | 578 |
| 172 | 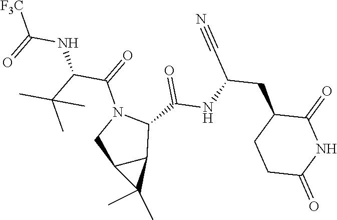 [SMILES: CC(C)(C)[C@H](NC(=O)C(F)(F)F)C(=O)N1C[C@H]2[C@@H]([C@H]1C(=O)N[C@H](C#N)C[C@@H]1CCC(=O)NC1=O)C2(C)C] | 528.2433 | 528 |
| 173 | 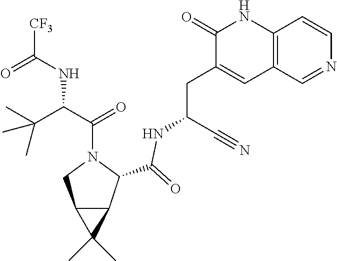 [SMILES: CC(C)(C)[C@H](NC(=O)C(F)(F)F)C(=O)N1C[C@H]2[C@@H]([C@H]1C(=O)N[C@@H](C#N)Cc1cc3cnccc3[nH]c1=O)C2(C)C] | 561.2437 | 561 |
| 174 | 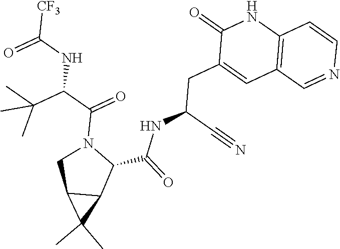 [SMILES: CC(C)(C)[C@H](NC(=O)C(F)(F)F)C(=O)N1C[C@H]2[C@@H]([C@H]1C(=O)N[C@H](C#N)Cc1cc3cnccc3[nH]c1=O)C2(C)C] | 561.2437 | 561 |
| 175 | 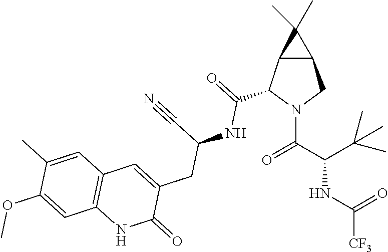 [SMILES: COc1cc2[nH]c(=O)c(C[C@@H](C#N)NC(=O)[C@@H]3[C@@H]4[C@H](CN3C(=O)[C@@H](NC(=O)C(F)(F)F)C(C)(C)C)C4(C)C)cc2cc1C] | 604.2746 | 604 |
| 176 | 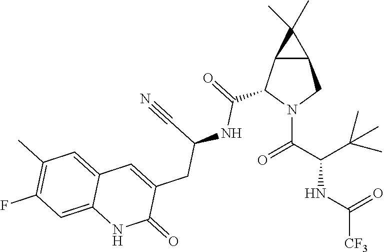 [SMILES: Cc1cc2cc(C[C@@H](C#N)NC(=O)[C@@H]3[C@@H]4[C@H](CN3C(=O)[C@@H](NC(=O)C(F)(F)F)C(C)(C)C)C4(C)C)c(=O)[nH]c2cc1F] | 592.2547 | 592 |
| 177 | 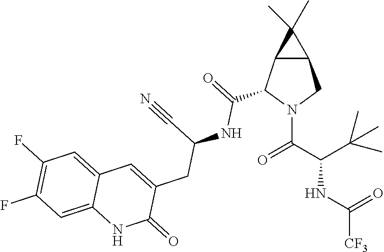 [SMILES: CC(C)(C)[C@H](NC(=O)C(F)(F)F)C(=O)N1C[C@H]2[C@@H]([C@H]1C(=O)N[C@H](C#N)Cc1cc3cc(F)c(F)cc3[nH]c1=O)C2(C)C] | 596.2296 | 596 |
| 178 | 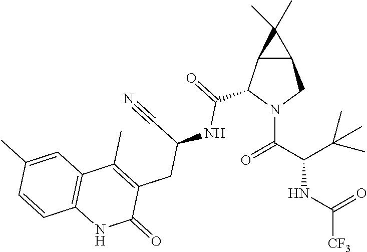 [SMILES: Cc1ccc2[nH]c(=O)c(C[C@@H](C#N)NC(=O)[C@@H]3[C@@H]4[C@H](CN3C(=O)[C@@H](NC(=O)C(F)(F)F)C(C)(C)C)C4(C)C)c(C)c2c1] | 588.2797 | 588 |
| 179 | 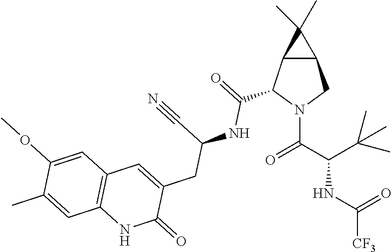 [SMILES: COc1cc2cc(C[C@@H](C#N)NC(=O)[C@@H]3[C@@H]4[C@H](CN3C(=O)[C@@H](NC(=O)C(F)(F)F)C(C)(C)C)C4(C)C)c(=O)[nH]c2cc1C] | 604.2746 | 604 |
| 180 | 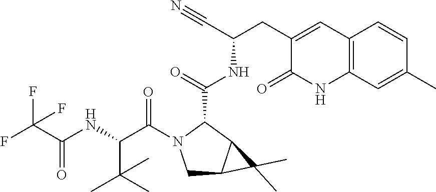 [SMILES: Cc1ccc2cc(C[C@@H](C#N)NC(=O)[C@@H]3[C@@H]4[C@H](CN3C(=O)[C@@H](NC(=O)C(F)(F)F)C(C)(C)C)C4(C)C)c(=O)[nH]c2c1] | 574.2641 | 574 |
| 181 | 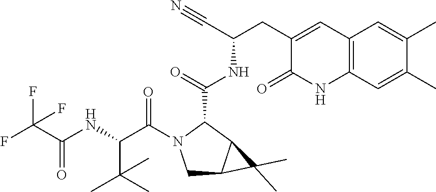 [SMILES: Cc1cc2cc(C[C@@H](C#N)NC(=O)[C@@H]3[C@@H]4[C@H](CN3C(=O)[C@@H](NC(=O)C(F)(F)F)C(C)(C)C)C4(C)C)c(=O)[nH]c2cc1C] | 588.2797 | 588 |
| 182 | 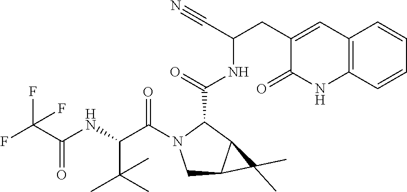 [SMILES: CC(C)(C)[C@H](NC(=O)C(F)(F)F)C(=O)N1C[C@H]2[C@@H]([C@H]1C(=O)NC(C#N)Cc1cc3ccccc3[nH]c1=O)C2(C)C] | 560.2485 | 560 |
| 183 | 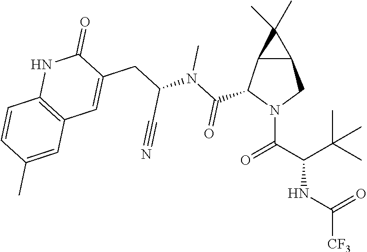 [SMILES: Cc1ccc2[nH]c(=O)c(C[C@@H](C#N)N(C)C(=O)[C@@H]3[C@@H]4[C@H](CN3C(=O)[C@@H](NC(=O)C(F)(F)F)C(C)(C)C)C4(C)C)cc2c1] | 588.2797 | 588 |
| 184 | 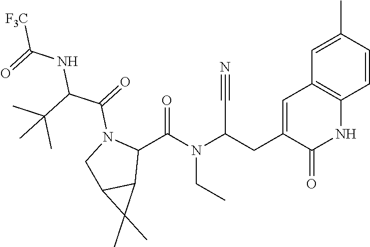 [SMILES: CCN(C(=O)C1C2C(CN1C(=O)C(NC(=O)C(F)(F)F)C(C)(C)C)C2(C)C)C(C#N)Cc1cc2cc(C)ccc2[nH]c1=O] | 602.2954 | 602 |
| 185 | 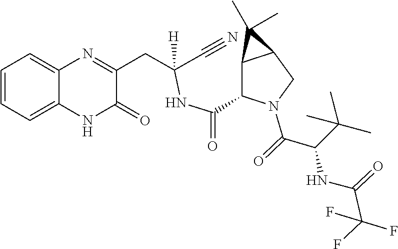 [SMILES: CC(C)(C)[C@H](NC(=O)C(F)(F)F)C(=O)N1C[C@H]2[C@@H]([C@H]1C(=O)N[C@H](C#N)Cc1nc3ccccc3[nH]c1=O)C2(C)C] | 561.2437 | 561 |
| 186 | 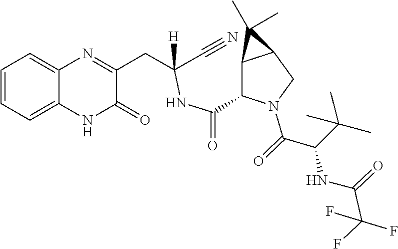 [SMILES: CC(C)(C)[C@H](NC(=O)C(F)(F)F)C(=O)N1C[C@H]2[C@@H]([C@H]1C(=O)N[C@@H](C#N)Cc1nc3ccccc3[nH]c1=O)C2(C)C] | 561.2437 | 561 |
| 187 | 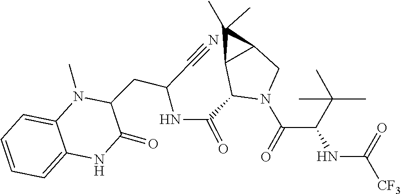 [SMILES: CN1c2ccccc2NC(=O)C1CC(C#N)NC(=O)[C@@H]1[C@@H]2[C@H](CN1C(=O)[C@@H](NC(=O)C(F)(F)F)C(C)(C)C)C2(C)C] | 577.275 | 577 |
| 188 | 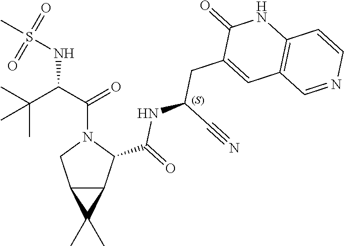 | 543.2389 | 543 |
| 189 | 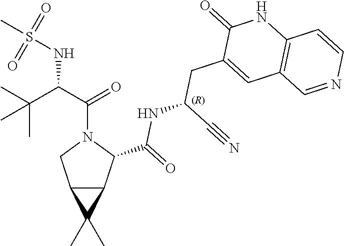 | 543.2389 | 543 |
| 190 | 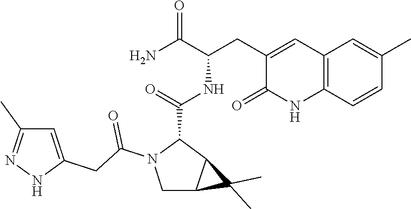 | 487.2457 | 487 |
| 191 | 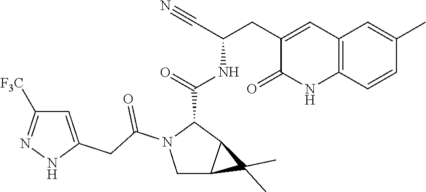 [SMILES: Cc1ccc2[nH]c(=O)c(C[C@@H](C#N)NC(=O)[C@@H]3[C@@H]4[C@H](CN3C(=O)Cc3cc(C(F)(F)F)n[nH]3)C4(C)C)cc2c1] | 541.2175 | 541 |
| 192 | 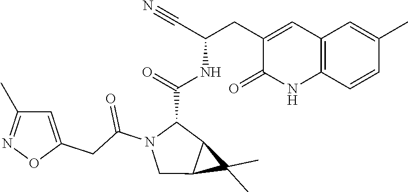 | 488.2298 | 488 |
| 193 | 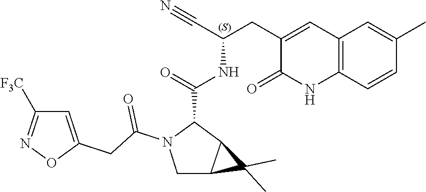 | 542.2015 | 542 |
| 194 | 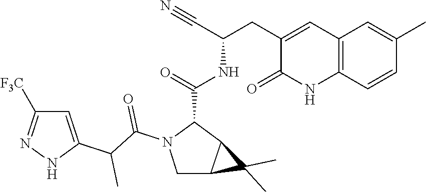 [SMILES: Cc1ccc2[nH]c(=O)c(C[C@@H](C#N)NC(=O)[C@@H]3[C@@H]4[C@H](CN3C(=O)C(C)c3cc(C(F)(F)F)n[nH]3)C4(C)C)cc2c1] | 555.2331 | 555 |
| 195 | 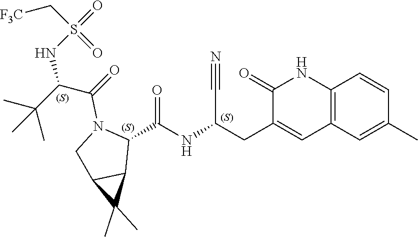 | 624.2468 | 624 |
| 196 | 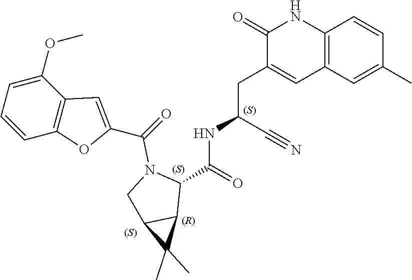 | 539.2294 | 539 |
| 197 | 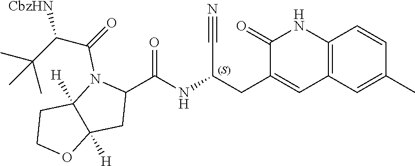 | 614.2978 | 614 |
| 198 | 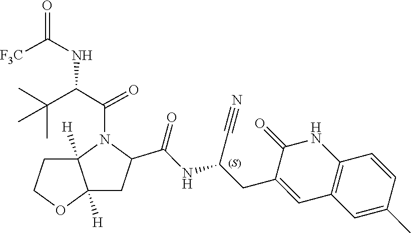 | 576.2433 | 576 |
| 199 | 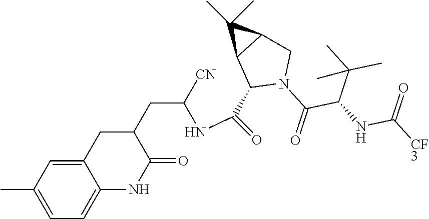 [SMILES: Cc1ccc2c(c1)CC(CC(C#N)NC(=O)[C@@H]1[C@@H]3[C@H](CN1C(=O)[C@@H](NC(=O)C(F)(F)F)C(C)(C)C)C3(C)C)C(=O)N2] | 576.2797 | 576 |
| 200 | 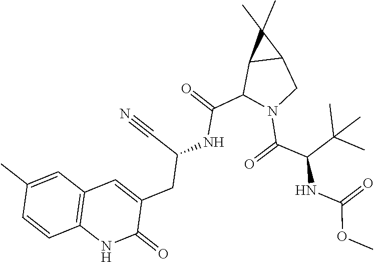 | 536.2873 | 536 |
| 201 | 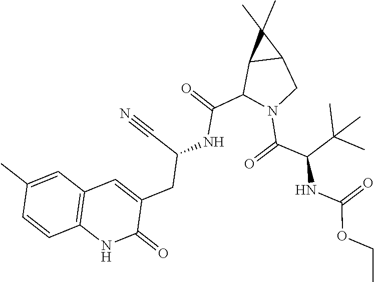 | 550.3029 | 550 |
| 202 | 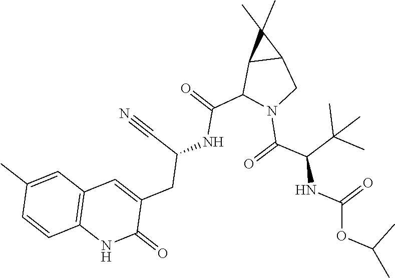 | 564.3186 | 564 |
| 203 | 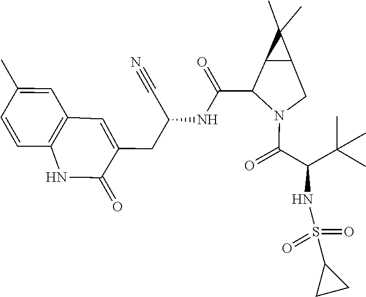 | 582.275 | 582 |
| 204 | 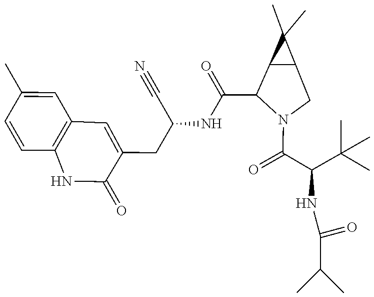 | 548.3237 | 548 |
| 205 | 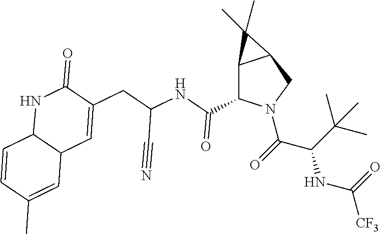 [SMILES: CC1=CC2C=C(CC(C#N)NC(=O)[C@@H]3[C@@H]4[C@H](CN3C(=O)[C@@H](NC(=O)C(F)(F)F)C(C)(C)C)C4(C)C)C(=O)NC2C=C1] | 588.2797 | 588 |
| 206 | 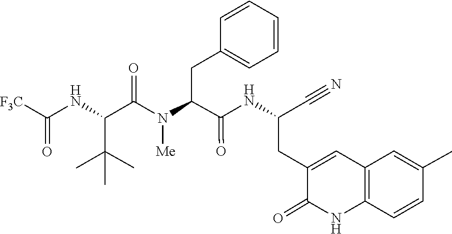 [SMILES: Cc1ccc2[nH]c(=O)c(C[C@@H](C#N)NC(=O)[C@H](Cc3ccccc3)N(C)C(=O)[C@@H](NC(=O)C(F)(F)F)C(C)(C)C)cc2c1] | 598.264 | 598 |
| 207 | 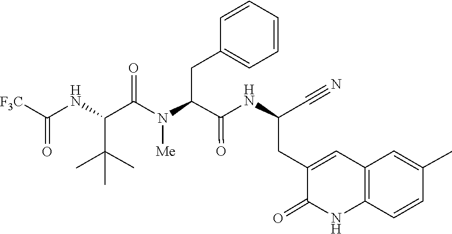 [SMILES: Cc1ccc2[nH]c(=O)c(C[C@H](C#N)NC(=O)[C@H](Cc3ccccc3)N(C)C(=O)[C@@H](NC(=O)C(F)(F)F)C(C)(C)C)cc2c1] | 598.264 | 599 |
| 208 | 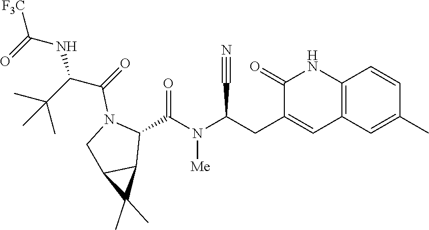 [SMILES: Cc1ccc2[nH]c(=O)c(C[C@H](C#N)N(C)C(=O)[C@@H]3[C@@H]4[C@H](CN3C(=O)[C@@H](NC(=O)C(F)(F)F)C(C)(C)C)C4(C)C)cc2c1] | 588.28 | 588 |
| 209 | 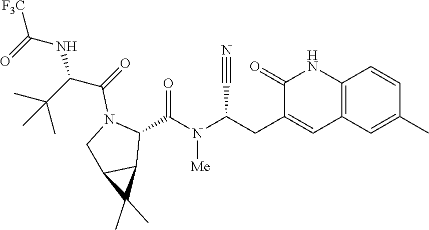 [SMILES: Cc1ccc2[nH]c(=O)c(C[C@@H](C#N)N(C)C(=O)[C@@H]3[C@@H]4[C@H](CN3C(=O)[C@@H](NC(=O)C(F)(F)F)C(C)(C)C)C4(C)C)cc2c1] | 588.28 | 588 |

### Synthesis of the Representative Examples of the Compound

### Example 1. (1R,2S,5S)—N-(1-Amino-3-(6-methyl-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (Compound 110)

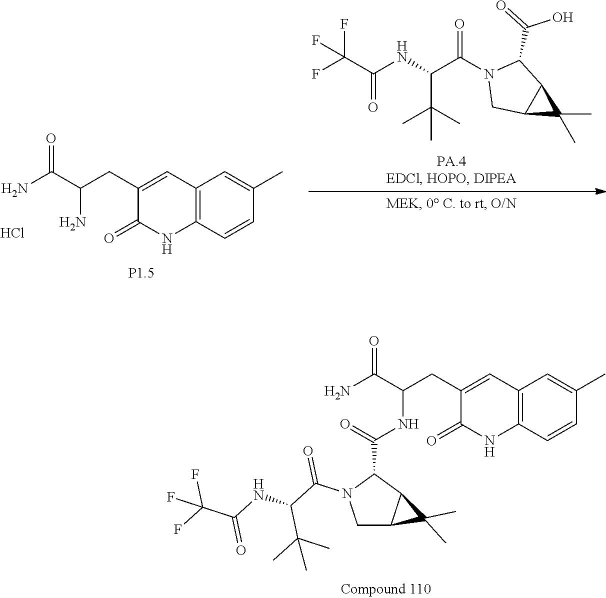 [SMILES: Cc1ccc2[nH]c(=O)c(CC(N)C(N)=O)cc2c1 ; Cl ; CC(C)(C)[C@H](NC(=O)C(F)(F)F)C(=O)N1C[C@H]2[C@@H]([C@H]1C(=O)O)C2(C)C ; Cc1ccc2[nH]c(=O)c(CC(NC(=O)[C@@H]3[C@@H]4[C@H](CN3C(=O)[C@@H](NC(=O)C(F)(F)F)C(C)(C)C)C4(C)C)C(N)=O)cc2c1]

To a solution of P1.5 (149 mg, 0.53 mmol), (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid (PA.4, 175 mg, 0.48 mmol) in butan-2-one (1.56 mL) was added 2-hydroxypyridine 1-oxide (13 mg, 0.12 mmol) and 1-(3-dimethylaminopropyl)-3-ethylcarbodiimide hydrochloride (118 mg, 0.62 mmol) at 0° C. N,N-Diisopropylethylamine (0.34 mL, 1.92 mmol) was added slowly to the reaction mixture and the resulting mixture was stirred for overnight. The residue was diluted with a solution of EtOAc/MTBE (1/1) and washed with 1N HCl, brine, saturated NaHCO_{3(aq)}, and brine. The organic layer was dried over MgSO₄₍ₛ₎, filtered, and then concentrated under reduced pressure. The residue was purified by silica gel column chromatography (0-11% methanol in dichloromethane) to give compound 110 (90 mg, 32%) as a solid. ¹H NMR (400 MHz, DMSO-d₆), δ: 11.81 (br. s, 1H), 11.72 (br. s, 1H), 9.45 (d, J=8.0 Hz, 1H), 9.36 (d, J=8.0 Hz, 1H), 8.55 (d, J=8.0 Hz, 1H), 8.31 (d, J=8.0 Hz, 1H), 7.64 (s, 1H), 7.60 (s, 1H), 7.37 (s, 1H), 7.30 (s, 1H), 7.28-7.24 (m, 3H), 7.18-7.16 (m, 2H), 7.08 (s, 1H), 7.06 (s, 1H), 6.95 (s, 1H), 4.50-4.32 (m, 4H), 4.12 (s, 1H), 4.08 (s, 1H), 3.99-3.97 (m, 1H), 3.80-3.78 (m, 1H), 3.61 (s, 1H), 3.58 (s, 1H), 3.17-3.15 (m, 1H), 2.93-2.91 (m, 1H), 2.83-2.81 (m, 1H), 2.59-2.53 (m, 1H), 2.30 (s, 3H), 2.29 (s, 3H), 1.90 (s, 1H), 1.50-1.47 (m, 1H), 1.30-1.28 (m, 1H), 1.14 (d, J=8.0 Hz, 1H), 0.99-0.92 (m, 21H), 0.89-0.92 (m, 16H); LCMS (ESI) m/z: calc. for C₂₉H₃₆F₃N₅O₅ 591.27. found, 592.2 [M+H]⁺.

### Example 2. (1R,2S,5S)—N-(1-Cyano-2-(6-methyl-2-oxo-1,2-dihydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 107)

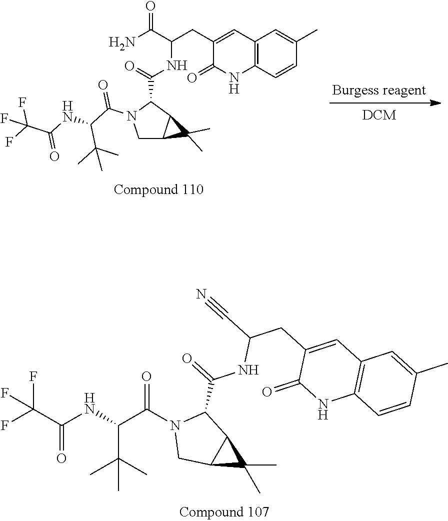 [SMILES: Cc1ccc2[nH]c(=O)c(CC(NC(=O)[C@@H]3[C@@H]4[C@H](CN3C(=O)[C@@H](NC(=O)C(F)(F)F)C(C)(C)C)C4(C)C)C(N)=O)cc2c1 ; Cc1ccc2[nH]c(=O)c(CC(C#N)NC(=O)[C@@H]3[C@@H]4[C@H](CN3C(=O)[C@@H](NC(=O)C(F)(F)F)C(C)(C)C)C4(C)C)cc2c1]

To a solution of Compound 110 (40 mg, 0.067 mmol) in DCM (0.34 mL) was added Burgess reagent (40 mg, 0.17 mmol), and the resulting mixture was stirred for 2 h. The residue was diluted with a solution of EtOAc/MTBE (1/1) and washed with saturated NaHCO_{3(aq)}, 1N HCl_{(aq)}, and brine. The organic layer was dried over MgSO₄₍ₛ₎, filtered, and concentrated under reduced pressure. The residue was purified by silica gel column chromatography (0-15% methanol in dichloromethane) to give compound 107 (20 mg, 52%) as a solid. ¹H NMR (400 MHz, DMSO-d₆), δ: 11.86 (s, 1H), 11.85 (s, 1H), 9.40-9.35 (m, 2H), 8.96 (d, J=8.0 Hz, 1H), 8.84 (d, J=8.0 Hz, 1H), 7.78 (d, J=4.8 Hz, 2H), 7.39 (d, J=6.8 Hz, 2H), 7.28 (d, J=8.0, 2H), 7.18 (d, J=8.0 Hz, 2H), 5.13-5.03 (m, 2H), 4.38-4.32 (m, 2H), 4.11 (d, J=5.2 Hz, 2H), 3.93-3.90 (m, 1H), 3.79-3.77 (m, 1H), 3.60-5.57 (m, 2H), 3.06-2.98 (m, 3H), 2.94-2.88 (m, 1H), 2.29 (s, 6H), 1.50-1.48 (m, 1H), 1.27-1.24 (m, 1H), 1.04 (d, J=7.6 Hz, 1H), 0.95-0.93 (m, 18H), 0.89-0.80 (m, 8H), 0.76 (s, 6H); LCMS (ESI) m/z: calc. for C₂₉H₃₄F₃N₅O₄ 573.26. found, 574.2 [M+H]⁺.

### Example 3. (1R,2S,5S)—N-(1-Amino-3-(7-fluoro-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (Compound 108)

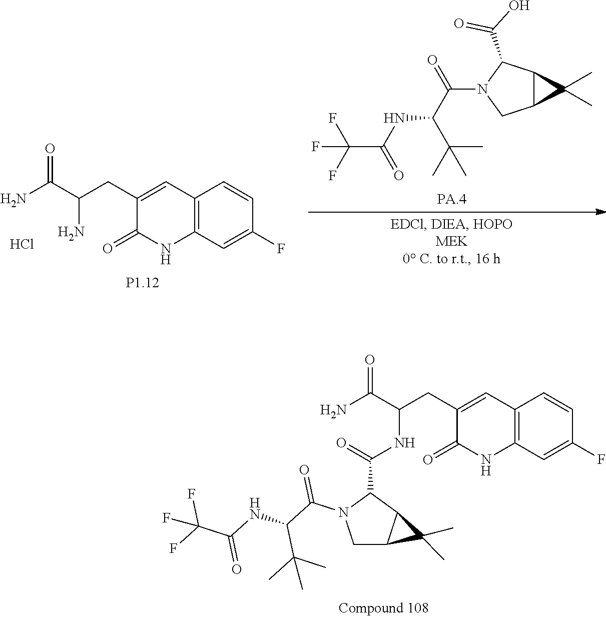

To a solution of (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid (PA.4, 100 mg, 0.274 mmol, 1.0 eq) and P1.12 (88.6 mg, 0.31 mmol, 1.13 eq) in methyl ethyl ketone (MEK, 1.1 mL) was added 2-hydroxypyridine-N-oxide (HOPO, 7.6 mg, 0.069 mmol, 0.25 eq). After cooled to 0° C., the reaction mixture was added N,N-diisopropylethylamine (DIEA, 0.14 mL, 0.82 mmol, 3.0 eq) and 1-ethyl-3-(3-dimethylaminopropyl)carbodiimide hydrochloride (EDCI, 63.1 mg, 0.33 mmol, 1.2 eq). After the reaction mixture was stirred at rt for 16 h, the reaction mixture was treated with water and extracted with ethyl acetate/methyl tert-butyl ether (1:1) for 3 times. The combined organic layers were washed with 1 M HCl_{(aq)} and brine, dried over Na₂SO₄₍ₛ₎, filtered, and concentrated in vacuo. The residue was purified by C-18 reversed-phase column chromatography (mobile phase A: water (with NH₄HCO₃), mobile phase B: methanol, UV: 214 and 254 nm, Flow rate: 35 mL/min, Gradient: 3-90% (% B)) to give compound 108 (87 mg, 53% yield) as a white solid. ¹H NMR (400 MHz, DMSO-d₆), δ: 11.95-11.88 (m, 1H), 9.43-9.36 (m, 1H), 8.50-8.28 (m, 1H), 7.74-7.60 (m, 2H), 7.26-6.96 (m, 4H), 4.55-4.33 (m, 2H), 4.14-4.12 (m, 1H), 3.98-3.78 (m, 1H), 3.67-3.59 (m, 1H), 3.17-2.54 (m, 2H), 1.50-1.30 (m, 1H), 1.15-0.79 (m, 16H); LCMS (ESI+) m/z: calc. for C₂₈H₃₃F₄N₅O₅ 595.24. found, 596.3 [M+H]⁺; HPLC purity: 100%, t_{R}=18.684 min.

### Example 4. (1R,2S,5S)—N-(1-Cyano-2-(7-fluoro-2-oxo-1,2-dihydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 106)

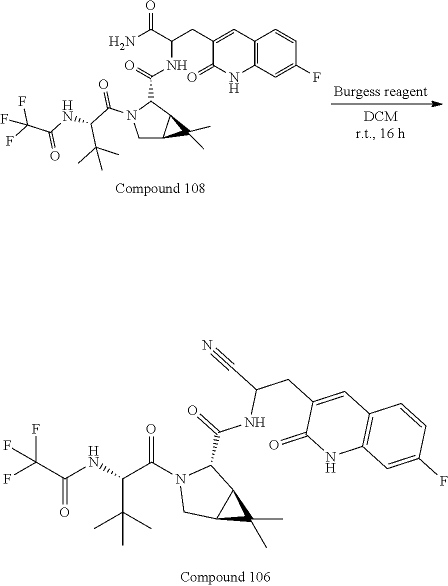

To a solution of compound 108 (51 mg, 0.086 mmol, 1.0 eq) in DCM (0.30 mL) was added methyl N-(triethylammoniumsulfonyl)carbamate (Burgess reagent, 51.0 mg, 0.214 mmol, 2.5 eq). After the solution was stirred at rt for 16 h, the mixture was treated with water and extracted with DCM 3 times. The combined organic layers were dried over Na₂SO₄₍ₛ₎, filtered, and concentrated in vacuo. The residue was purified by C-18 reversed-phase column chromatography (mobile phase A: water (with NH₄HCO₃), mobile phase B: acetonitrile, UV: 214 and 254 nm, Flow rate: 35 mL/min, Gradient: 3-90% (% B)) to give compound 106 (30 mg, 61% yield) as a white solid. ¹H NMR (400 MHz, DMSO-d₆), δ: 12.04 (s, 1H), 9.39 (d, J=6.8 Hz, 1H), 8.99-8.88 (m, 1H), 7.91 (s, 1H), 7.76-7.70 (m, 1H), 7.09-7.02 (m, 2H), 5.17-5.06 (m, 1H), 4.41-4.34 (m, 1H), 4.15 (s, 1H), 3.96-3.79 (m, 1H), 3.65-3.63 (m, 1H), 3.10-2.91 (m, 2H), 1.54-1.32 (m, 1H), 1.10-1.01 (m, 1H), 1.00-0.79 (m, 15H); LCMS (ESI+) m/z: calc. for C₂₈H₃₁F₄N₅O₄ 577.23. found, 578.3 [M+H]⁺; HPLC purity: 100%, t_{R}=21.288 min.

### Example 5. (1R,2S,5S)—N-(1-Amino-3-(6-methoxy-2-oxo-1,2-dihydroquinolin-3-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (Compound 111)

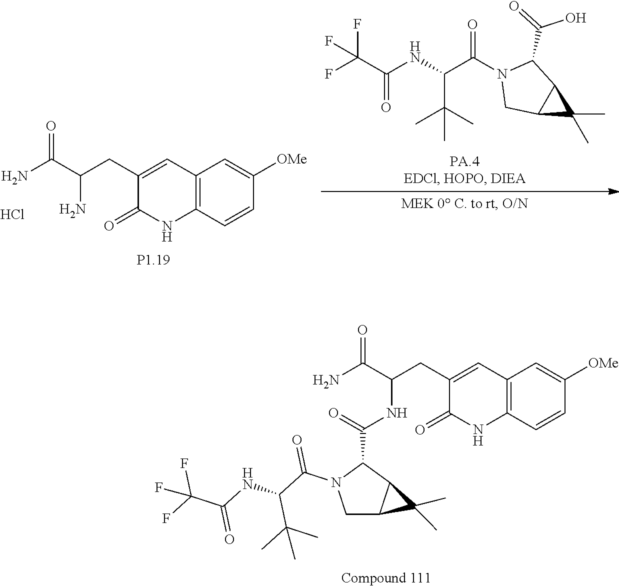 [SMILES: COc1ccc2[nH]c(=O)c(CC(N)C(N)=O)cc2c1 ; Cl ; CC(C)(C)[C@H](NC(=O)C(F)(F)F)C(=O)N1C[C@H]2[C@@H]([C@H]1C(=O)O)C2(C)C ; COc1ccc2[nH]c(=O)c(CC(NC(=O)[C@@H]3[C@@H]4[C@H](CN3C(=O)[C@@H](NC(=O)C(F)(F)F)C(C)(C)C)C4(C)C)C(N)=O)cc2c1]

To a solution of P1.19 (200 mg, 0.67 mmol) and (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid (PA.4, 222 mg, 0.61 mmol) in butan-2-one (2.0 mL) was added 2-hydroxypyridine 1-oxide (17 mg, 0.15 mmol) and 1-(3-dimethylaminopropyl)-3-ethylcarbodiimide hydrochloride (151 mg, 0.79 mmol) at 0° C. After N,N-diisopropylethylamine (0.42 mL, 2.4 mmol) was added slowly to the reaction mixture, the resulting mixture was stirred for 16 h. The reaction was concentrated under reduced pressure. The residue was purified by C18 reverse phase column (0-100% MeOH in water) to give Compound 111 (190 mg, 56%) as a white solid. ¹H NMR (400 MHz, DMSO-d₆), δ: 11.78-11.68 (m, 1H), 9.42-9.34 (m, 1H), 8.55-8.33 (m, 1H), 7.64-7.59 (m, 1H), 7.21-7.02 (m, 5H), 6.98-6.92 (m, 1H), 4.49-4.25 (m, 2H), 4.09-4.04 (m, 1H), 3.98-3.76 (m, 1H), 3.72 (s, 3H), 3.58-3.55 (m, 1H), 3.13-2.54 (m, 2H), 1.47-1.27 (m, 1H), 1.12 (d, J=7.8 Hz, 0.5H), 0.93-0.74 (m, 15.5H); LCMS (ESI+) m/z: calc. for C₂₉H₃₆F₃N₅O₆ 607.26. found, 608.4 [M+H]⁺; HPLC purity: 91.1%, t_{R}=18.149 min.

### Example 6. (1R,2S,5S)—N-(1-Cyano-2-(6-methoxy-2-oxo-1,2-dihydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 109)

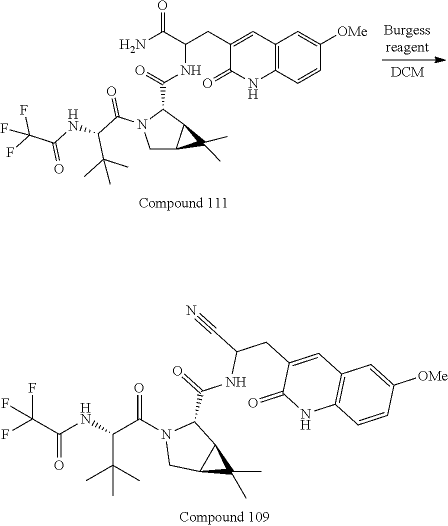 [SMILES: COc1ccc2[nH]c(=O)c(CC(NC(=O)[C@@H]3[C@@H]4[C@H](CN3C(=O)[C@@H](NC(=O)C(F)(F)F)C(C)(C)C)C4(C)C)C(N)=O)cc2c1 ; COc1ccc2[nH]c(=O)c(CC(C#N)NC(=O)[C@@H]3[C@@H]4[C@H](CN3C(=O)[C@@H](NC(=O)C(F)(F)F)C(C)(C)C)C4(C)C)cc2c1]

To a solution of compound 111 (181 mg, 0.300 mmol) in DCM (2.0 mL) was added Burgess reagent (177 mg, 0.740 mmol). After the reaction mixture was stirred at rt for 2 h, the solution was concentrated under reduced pressure. The residue was purified by C18 reverse phase column (0-100% MeOH in water) to give compound 109 (154 mg, 87%) as a yellow solid. ¹H NMR (400 MHz, DMSO-d₆), δ: 11.87 (br. s, 1H), 9.38 (br. s, 1H), 8.99-8.88 (m, 1H), 7.83 (s, 1H), 7.26-7.12 (m, 3H), 5.17-5.06 (m, 1H), 4.38-4.36 (m, 1H), 4.15-4.14 (m, 1H), 3.96-3.76 (m, 1H), 3.74 (s, 3H), 3.64-3.60 (m, 1H), 3.08-2.92 (m, 2H), 1.53-1.30 (m, 1H), 1.09-0.79 (m, 16H); LCMS (ESI+) m/z: calc. for C₂₉H₃₄F₃N₅O₅ 589.25. found, 590.4 [M+H]⁺; HPLC purity: 98.4%, t_{R}=20.770 min.

### Example 7. (1R,2S,5S)—N-(1-Cyano-2-(6-methyl-2-oxo-1,2-dihydroquinolin-3-yl)ethyl)-3-((S)-3,3-dimethyl-2-(methylsulfonamido)butanoyl)-6,6-dimethyl-3-azabicyclo[3.1.0]hexane-2-carboxamide (Compound 102)

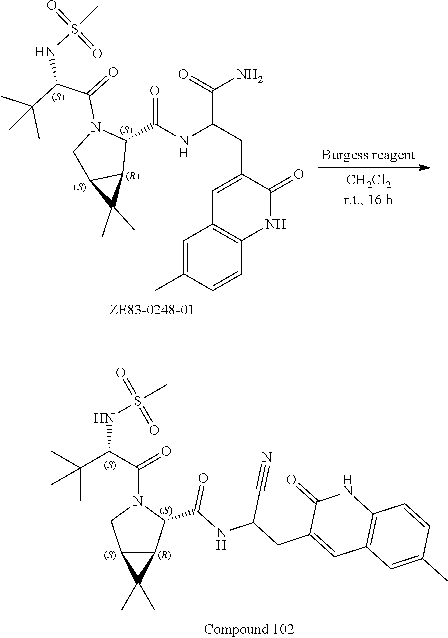

To a solution of P1.20 (305.0 mg, 0.532 mmol, 1.0 eq) in DCM (5.3 mL) was added Burgess reagent (316.7 mg, 1.33 mmol, 2.5 eq), and the resulting mixture was stirred at rt for 16 h. The solution was treated with water, extracted with DCM three times. The combined organic layers were washed with brine, dried over Na₂SO₄₍ₛ₎, filtered and concentrated in vacuo to give the residue. The residue was purified by C-18 reversed-phase column chromatography (mobile phase A: water (with 1% NH₄HCO₃), mobile phase B: acetonitrile, UV: 214 and 254 nm, Flow rate: 60 mL/min, Gradient: 3-90% (% B)) to give compound 102 (263 mg, 89% yield) as a white solid. ¹H NMR (400 MHz, DMSO-d₆), δ: 11.87-11.85 (m, 1H), 9.03-8.95 (m, 1H), 7.83-7.82 (m, 1H), 7.42 (d, J=8.0 Hz, 1H), 7.34-7.28 (m, 1H), 7.24-7.18 (m, 1H), 7.04-6.97 (m, 1H), 5.18-5.04 (m, 1H), 4.19-4.17 (m, 1H), 3.89-3.60 (m, 3H), 3.12-2.90 (m, 2H), 2.85-2.84 (m, 3H), 2.33-2.32 (m, 3H), 1.56-1.21 (m, 1H), 1.15-0.81 (m, 16H); LCMS (ESI) m/z calc. for C₂₈H₃₇N₅O₅S 555.25. found, 556.3 [M+H]⁺.

### Example 8. (1R,2S,5S)—N-(1-Cyano-2-(7-methyl-2-oxo-1,2-dihydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 146)

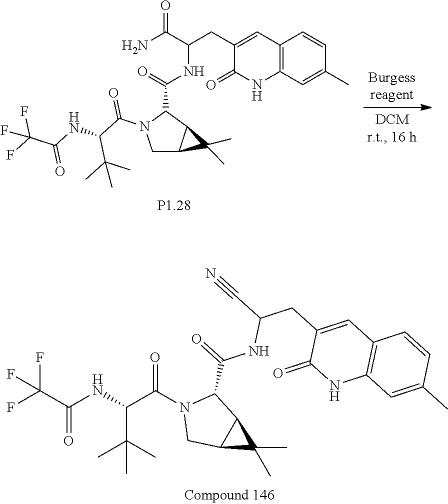

To a solution of P1.28 (210 mg, 0.35 mmol, 1.0 eq) in DCM (2.3 mL) was added Burgess reagent (211 mg, 0.89 mmol, 2.5 eq). The resulting mixture was stirred at rt for 16 h. The mixture was concentrated under reduced pressure and purified by C-18 reversed-phase column chromatography (0-100% MeOH in water containing 1% NH₄HCO₃) to give compound 146 (162.4 mg, 79% yield) as a yellow solid. ¹H NMR (400 MHz, DMSO-d₆), δ: 11.89-11.87 (m, 2H), 9.41-9.38 (m, 2H), 8.99-8.87 (m, 2H), 7.84 (s, 2H), 7.54-7.51 (m, 2H), 7.09 (s, 2H), 7.01-6.99 (m, 2H), 5.16-5.05 (m, 2H), 4.40-4.36 (m, 2H), 4.15-4.14 (m, 2H), 3.96-3.79 (m, 2H), 3.65-3.60 (m, 2H), 3.06-2.90 (m, 4H), 2.36 (s, 6H), 1.52-1.30 (m, 2H), 1.09 (d, J=7.8 Hz, 1H), 0.98-0.79 (m, 31H); LCMS (ESI) m/z calc. for C₂₉H₃₄F₃N₅O₄ 573.26. found, 574.2 [M+H]⁺; HPLC purity: 99.3%, t_{R}=21.753 min.

### Example 9. (1R,2S,5S)—N-(1-cyano-2-(6,7-dimethyl-2-oxo-1,2-dihydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 145)

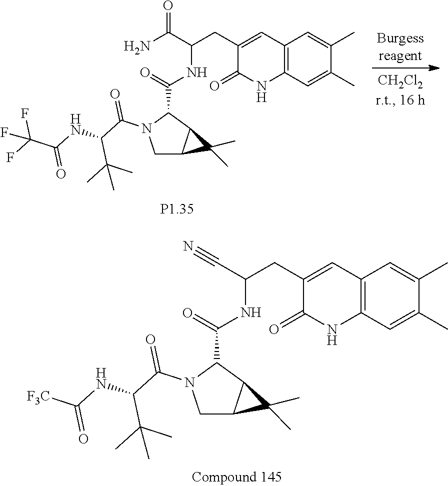

To a solution of P1.35 (340 mg, 0.56 mmol, 1.0 eq) in DCM (3.7 mL) was added Burgess reagent (334 mg, 1.40 mmol, 2.5 eq). The resulting mixture was stirred at rt for 16 h. The mixture was concentrated under reduced pressure and purified by C-18 reversed-phase column chromatography (0-100% MeOH in water containing 1% NH₄HCO₃) to give compound 145 (272.2 mg, 82% yield) as a yellow solid. ¹H NMR (400 MHz, DMSO-d₆), δ: 11.81-11.80 (m, 2H), 9.42-9.37 (m, 2H), 8.99-8.85 (m, 2H), 7.77-7.76 (m, 2H), 7.38-7.36 (m, 2H), 7.07-7.06 (m, 2H), 5.13-5.03 (m, 2H), 4.40-4.35 (m, 2H), 4.14-4.13 (m, 2H), 3.97-3.79 (m, 2H), 3.63-3.59 (m, 2H), 3.06-2.89 (m, 4H), 2.26-2.22 (m, 12H), 1.53-1.15 (m, 3H), 0.98-0.84 (m, 25H), 0.78 (s, 6H); LCMS (ESI) m/z calc. for C₃₀H₃₆F₃N₅O₄ 587.27. found, 588.2 [M+H]⁺; HPLC purity: 99.5%, t_{R}=22.55 min.

### Example 10. (1R,2S,5S)—N-(1-Cyano-2-(2-oxo-1,2-dihydropyridin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 101)

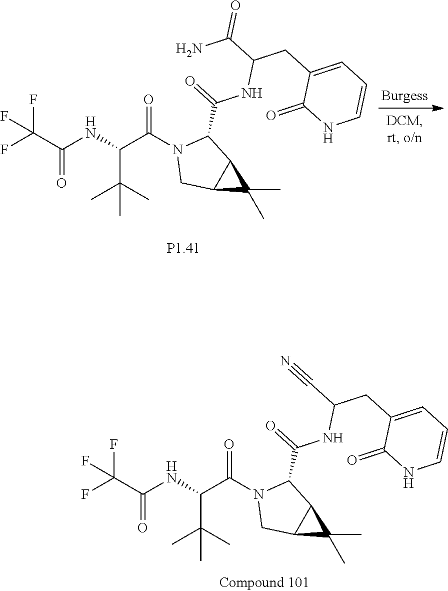 [SMILES: CC(C)(C)[C@H](NC(=O)C(F)(F)F)C(=O)N1C[C@H]2[C@@H]([C@H]1C(=O)NC(Cc1ccc[nH]c1=O)C(N)=O)C2(C)C ; CC(C)(C)[C@H](NC(=O)C(F)(F)F)C(=O)N1C[C@H]2[C@@H]([C@H]1C(=O)NC(C#N)Cc1ccc[nH]c1=O)C2(C)C]

To a solution of P1.41 (150 mg, 0.28 mmol) in DCM (2.0 mL) was added Burgess reagent (167 mg, 0.7 mmol), and the resulting mixture was stirred for overnight. The mixture was concentrated under reduced pressure. The residue was purified by C-18 reverse phase chromatography column chromatography (0-70% MeOH in water) and preparative HPLC (0-50% ACN in water) to give compound 101 (15.3 mg, 11%) as a white solid. ¹H NMR (400 MHz, MeOD-d4), δ: 7.60-7.57 (m, 2H), 7.42-7.38 (m, 2H), 6.40-6.35 (m, 2H), 5.14-5.11 (m, 2H), 4.57-4.56 (m, 2H), 4.28 (s, 2H), 4.07-3.98 (m, 2H), 3.85-3.81 (m, 2H), 3.14-2.97 (m, 4H), 1.63-1.52 (m, 2H), 1.34-1.22 (m, 2H), 1.07-1.03 (m, 24H), 0.91-0.90 (d, J=2 Hz, 6H).; LCMS (ESI) m/z calc. for C₂₄H₃₀F₃N₅O₄ 509.22. found, 510.3 [M+H]⁺. HPLC purity: 100%, t_{R}=17.942 min.

### Example 11. Chiral separation of (1R,2S,5S)—N-(1-cyano-2-(6-methyl-2-oxo-1,2-dihydroquinolin-3-yl)ethyl)-3-((S)-3,3-dimethyl-2-(methylsulfonamido)butanoyl)-6,6-dimethyl-3-azabicyclo[3.1.0]hexane-2-carboxamide (Compound 102): (1R,2S,5S)—N-[(1S)-1-cyano-2-(6-methyl-2-oxo-1H-quinolin-3-yl)ethyl]-3-[(2S)-2-(methanesulfonamido)-3,3-dimethyl-butanoyl]-6,6-dimethyl-3-azabicyclo[3.1.0]hexane-2-carboxamide (Compound 147) and (1R,2S,5S)—N-[(1R)-1-cyano-2-(6-methyl-2-oxo-1H-quinolin-3-yl)ethyl]-3-[(2S)-2-(methanesulfonamido)-3,3-dimethyl-butanoyl]-6,6-dimethyl-3-azabicyclo[3.1.0]hexane-2-carboxamide (Compound 148)

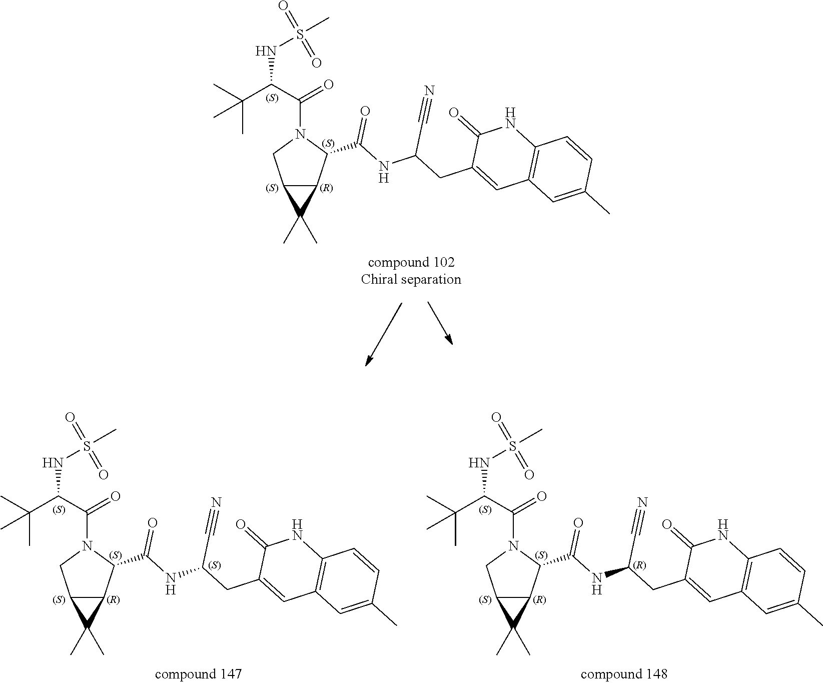

### Conditions

| | |
|---|---|
| Instrument | WATERS SFC-80 |
| Wavelength | 230 nm |
| Column | CHIRALPAK IH (30 mm * 250 mm, 10 μm) |
| Flow Rate | 60 g/min |
| Solvent: | CO₂:IPA = 65:35 |

### Result

| sample | ee (%), 230 nm | amount | |
|---|---|---|---|
| Compound 102 | — | 132.7 | mg |
| Compound 147 | 99.2 | 61.2 | mg |
| Compound 148 | 100 | 56.4 | mg |

### Example 12. Chiral separation of (1R,2S,5S)—N-(1-cyano-2-(6-methyl-2-oxo-1,2-dihydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 107): (1S,2S,5S)—N-[(1S)-1-cyano-2-(6-methyl-2-oxo-1H-quinolin-3-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 113), (1S,2S,5S)—N-[(1R)-1-cyano-2-(6-methyl-2-oxo-1H-quinolin-3-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 114)

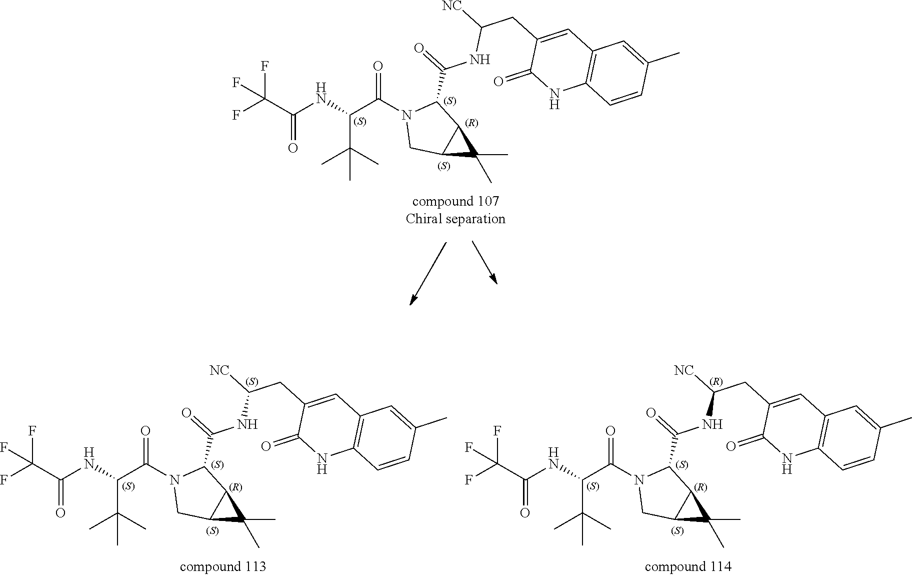

### Conditions

| | |
|---|---|
| Instrument | WATERS SFC-80 |
| Wavelength | 230 nm |
| Column | CHIRALPAK IC (30 mm * 250 mm, 10 μm) |
| Flow Rate | 60 g/min |
| Solvent: | CO₂:Ethanol = 80:20 |

### Result

| Sample | ee (%), 230 nm | amount | |
|---|---|---|---|
| Compound 107 | — | 153.4 | mg |
| Compound 113 | 96.7 | 76 | mg |
| Compound 114 | 100 | 40 | mg |

Compound 113: ¹H NMR (400 MHz, DMSO-d₆), δ: 11.86 (s, 1H), 9.35 (d, J=8.4 Hz, 1H), 8.84 (d, J=7.4 Hz, 1H), 7.81 (s, 1H), 7.43 (s, 1H), 7.32 (d, J=8.6 Hz, 1H), 7.22 (d, J=8.2 Hz, 1H), 5.10 (q, J=7.6 Hz, 1H), 4.41 (d, J=8.6 Hz, 1H), 4.15 (s, 1H), 3.96 (dd, J=10.5, 5.4 Hz, 1H), 3.69-3.57 (m, 1H), 3.05 (d, J=7.9 Hz, 2H), 2.33 (s, 3H), 1.59-1.45 (m, 1H), 1.09 (d, J=7.6 Hz, 1H), 0.97 (s, 9H), 0.91 (s, 3H), 0.80 (s, 3H). δ LCMS (ESI) m/z calc. for C₂₉H₃₄F₃N₅O₄ 573.26. found, 574.2 [M+H]⁺.

Compound 114: ¹H NMR (400 MHz, DMSO-d₆), δ: 11.86 (s, 1H), 9.36 (d, J=8.8 Hz, 1H), 8.97 (d, J=8 Hz, 1H), 7.81 (s, 1H), 7.42 (s, 1H), 7.33 (d, J=8.8 Hz, 1H), 7.21 (d, J=8.2 Hz, 1H), 5.13 (q, J=7.6 Hz, 1H), 4.371 (d, J=8.4 Hz, 1H), 4.15 (s, 1H), 3.83 (dd, J=10.5, 5.4 Hz, 1H), 3.62 (d, J=10.4, 1H), 3.06-2.95 (m, 2H), 2.32 (s, 3H), 1.30-1.29 (m, 1H), 0.99 (s, 9H), 0.89 (d, J=7.6 Hz, 1H), 0.85 (s, 3H), 0.79 (s, 3H). LCMS (ESI) m/z calc. for C₂₉H₃₄F₃N₅O₄ 573.26. found, 574.2 [M+H]⁺.

### Example 13. (1R,2S,5S)—N-[(1S)-1-Cyano-2-(3-oxo-4H-quinoxalin-2-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 185) and (1R,2S,5S)—N-[(1R)-1-cyano-2-(3-oxo-4H-quinoxalin-2-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 186)

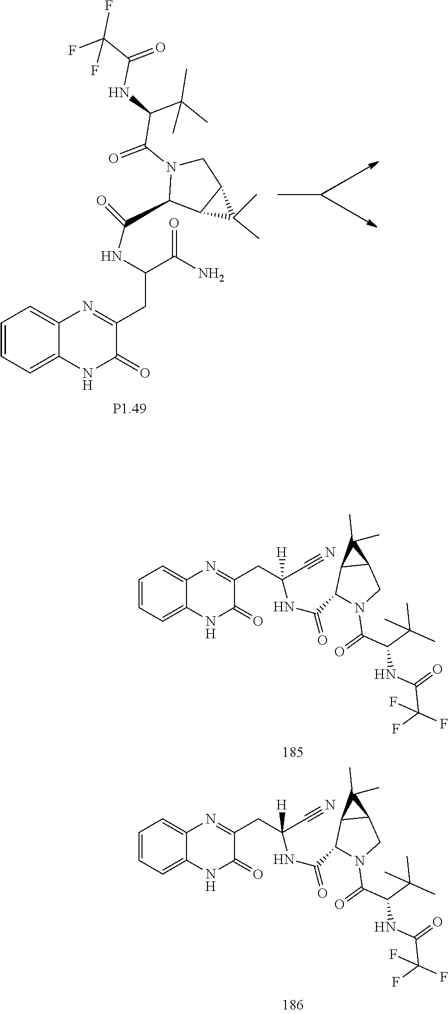 [SMILES: CC(C)(C)[C@H](NC(=O)C(F)(F)F)C(=O)N1C[C@H]2[C@@H]([C@H]1C(=O)NC(Cc1nc3ccccc3[nH]c1=O)C(N)=O)C2(C)C ; CC(C)(C)[C@H](NC(=O)C(F)(F)F)C(=O)N1C[C@H]2[C@@H]([C@H]1C(=O)N[C@H](C#N)Cc1nc3ccccc3[nH]c1=O)C2(C)C ; CC(C)(C)[C@H](NC(=O)C(F)(F)F)C(=O)N1C[C@H]2[C@@H]([C@H]1C(=O)N[C@@H](C#N)Cc1nc3ccccc3[nH]c1=O)C2(C)C]

To a solution of (1R,2S,5S)—N-[2-amino-2-oxo-1-[(3-oxo-4H-quinoxalin-2-yl)methyl]ethyl]-3-[(2S)-3,3-dimethyl-2-[(2,2,2-tri fluoroacetyl)amino]butanoyl]-6,6-dimethyl-3-azabicyclo[3.1.0]hexane-2-carboxamide (P1.49) in 5 ml DCM Burgess reagent (195 mg, 2.5 eq) was added. Reaction mixture was stirred at rt for 15 h, washed with water, saturated NaHCO₃ and 5% HCl, dried over Na₂SO₄, filtered, and concentrated under reduced pressure. The residue was purified by silica gel column chromatography (n-hexane/EtOAc 1.5:1, next n-hexane/EtOAc 1:1.5) to give 2 isomers 185 (402 mg, 20%) and 186 (415 mg, 20.7%). 185: ¹H NMR (400 MHz, DMSO) δ 12.51 (s, 1H), 9.37 (d, J=8.4 Hz, 1H), 9.01 (d, J=7.9 Hz, 1H), 7.74-7.69 (m, 1H), 7.53 (t, J=7.7 Hz, 1H), 7.30 (t, J=7.7 Hz, 2H), 5.38 (q, J=7.7 Hz, 1H), 4.39 (t, J=7.9 Hz, 1H), 4.18 (d, J=4.5 Hz, 1H), 3.82 (dd, J=10.4, 5.3 Hz, 1H), 3.64 (d, J=10.4 Hz, 1H), 3.46 (dd, J=16.3, 7.0 Hz, 1H), 3.23 (dd, J=16.3, 8.1 Hz, 1H), 3.15-3.04 (m, 1H), 1.37-1.22 (m, 2H), 1.12 (d, J=7.6 Hz, 1H), 0.98 (d, J=5.6 Hz, 9H), 0.86 (s, 3H), 0.79 (s, 3H). 186: ¹H NMR (400 MHz, DMSO) δ 12.51 (s, 1H), 9.37 (d, J=8.4 Hz, 1H), 8.96 (d, J=7.3 Hz, 1H), 7.76 (d, J=7.9 Hz, 1H), 7.53 (t, J=7.7 Hz, 1H), 7.30 (t, J=8.4 Hz, 2H), 4.40 (d, J=8.2 Hz, 1H), 4.18 (s, 1H), 3.92 (dd, J=10.3, 5.5 Hz, 1H), 3.70-3.61 (m, 1H), 3.44 (dd, J=16.4, 8.2 Hz, 1H), 3.26 (dd, J=16.4, 6.9 Hz, 1H), 1.58-1.50 (m, 1H), 1.25 (d, J=7.6 Hz, 1H), 0.97 (d, J=2.9 Hz, 12H), 0.81 (s, 3H).

### Example 14. (1R,2S,5S)—N-[1-Cyano-2-(1-methyl-3-oxo-2,4-dihydroquinoxalin-2-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 187)

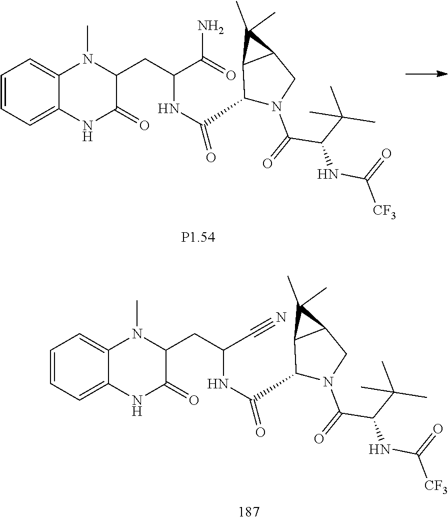 [SMILES: CN1c2ccccc2NC(=O)C1CC(NC(=O)[C@@H]1[C@@H]2[C@H](CN1C(=O)[C@@H](NC(=O)C(F)(F)F)C(C)(C)C)C2(C)C)C(N)=O ; CN1c2ccccc2NC(=O)C1CC(C#N)NC(=O)[C@@H]1[C@@H]2[C@H](CN1C(=O)[C@@H](NC(=O)C(F)(F)F)C(C)(C)C)C2(C)C]

To a solution of P1.54 (500 mg, 0.84 mmol) in 20 ml DCM Burgess reagent (601 mg, 2.52 mmol) was added. Reaction mixture was stirred at rt for 15 h, washed with water, saturated NaHCO₃ and 5% HCl, dried over Na₂SO₄, filtered, and concentrated under reduced pressure. The residue was purified by prep RP-HPLC on a C18 column eluting with a gradient MeCN-H₂O+0.1% TFA. Yield of compound 187: 23 mg, 5%.

### Example 15. (1R,2S,5S)—N—((S)-1-Cyano-2-(6-methyl-2-oxo-1,2-dihydroquinolin-3-yl)ethyl)-3-((S)-3,3-dimethyl-2-((4-methylthiazol-2-yl)amino)butanoyl)-6,6-dimethyl-3-azabicyclo[3.1.0]hexane-2-carboxamide (Compound 157)

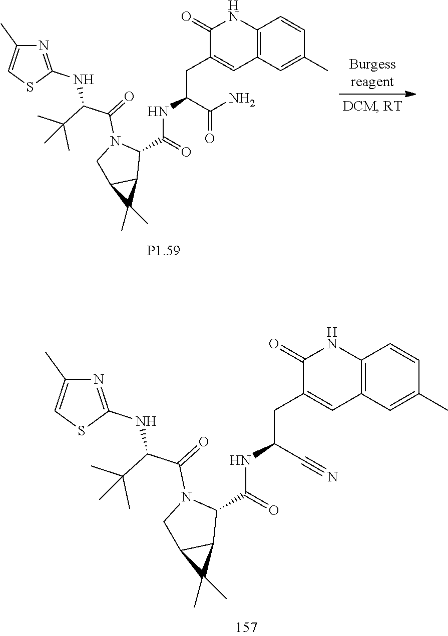

To a solution of P1.59 (40 mg, 0.0674 mmol, 1.0 eq) in DCM (1.5 mL) was added methyl N-(triethylammoniumsulfonyl)carbamate (40 mg, 0.169 mmol, 2.5 eq). The resulting mixture was stirred at rt for 16 h. Then the reaction mixture was concentrated to get crude residue. The residue was purified with C-18 reversed-phase column chromatography (mobile phase A: water, mobile phase B: methanol, UV: 214 and 254 nm, Flow rate: 35 mL/min, Gradient: 70-80% (% B)) to give compound 157 (25 mg, 64% yield) as a white solid. ¹H NMR (400 MHz, MeOD-d₄), δ: 7.88 (s, 1H), 7.44 (d, J=0.8 Hz, 1H), 7.35-7.34 (m, 1H), 7.23 (d, J=8.4 Hz, 1H), 6.07 (s, 1H), 5.21-5.18 (m, 1H), 4.44 (s, 1H), 4.23 (s, 1H), 4.15 (d, J=10.4 Hz, 1H), 4.07-4.04 (m, 1H), 3.24-3.14 (m, 2H), 2.39 (s, 3H), 2.13 (s, 3H), 1.58 (dd, J=7.6, 5.2 Hz, 1H), 1.19 (d, J=7.6 Hz, 1H), 0.99 (s, 9H), 0.96 (s, 3H), 0.80 (s, 3H); LCMS (ESI): m/z 575.6 [M+H]⁺.

### Example 16. (1R,2S,5S)—N-(1-Cyano-2-(6-methyl-2-oxo-1,2-dihydroquinolin-3-yl)ethyl)-3-((S)-3,3-dimethyl-2-((4-(trifluoromethyl)pyrimidin-2-yl)amino)butanoyl)-6,6-dimethyl-3-azabicyclo[3.1.0]hexane-2-carboxamide (Compound 156)

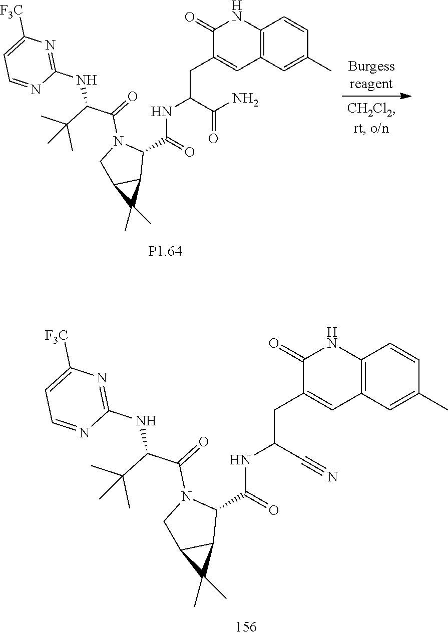 [SMILES: Cc1ccc2[nH]c(=O)c(CC(NC(=O)[C@@H]3[C@@H]4[C@H](CN3C(=O)[C@@H](Nc3nccc(C(F)(F)F)n3)C(C)(C)C)C4(C)C)C(N)=O)cc2c1 ; Cc1ccc2[nH]c(=O)c(CC(C#N)NC(=O)[C@@H]3[C@@H]4[C@H](CN3C(=O)[C@@H](Nc3nccc(C(F)(F)F)n3)C(C)(C)C)C4(C)C)cc2c1]

To a solution of P1.64 (83.0 mg, 0.115 mmol, 1.0 eq) in DCM (3.0 mL) was added methyl N-(triethylammoniumsulfonyl)carbamate (77.0 mg, 0.323 mmol) the resulting mixture stirred at rt for overnight. Then reaction mixture was quenched by water and extracted with DCM. The combined organic layers were dried over Na₂SO₄₍ₛ₎, filtered, and concentrated to obtain crude residue. The residue was purified with C-18 reversed-phase column chromatography to give desired product 156 as a white solid (45 mg, 56% yield). ¹H NMR (400 MHz, MeOD-d₄), δ: 8.52 (d, J=4.8 Hz 2H), 7.89 (s, 1H), 7.87 (s, 1H), 7.47-7.24 (m, 6H), 6.96-6.93 (m, 2H), 5.24-5.18 (m, 2H), 4.57 (br. s, 2H), 4.23 (s, 1H), 4.22 (s, 1H), 4.19-3.93 (m, 4H), 3.31-3.03 (m, 2H), 2.40 (s, 3H), 2.39 (s, 3H), 1.34-1.21 (m, 3H), 1.09-0.86 (m, 31H); LRMS (ESI): 624.2 [M+H]⁺.

### Example 17. 3-[3,3-Dimethyl-2-(2-methyl-pyrimidin-4-ylamino)-butyryl]-6,6-dimethyl-3-aza-bicyclo[3.1.0]hexane-2-carboxylic acid [1-cyano-2-(6-methyl-2-oxo-1,2-dihydro-quinolin-3-yl)-ethyl]-amide (Compound 153)

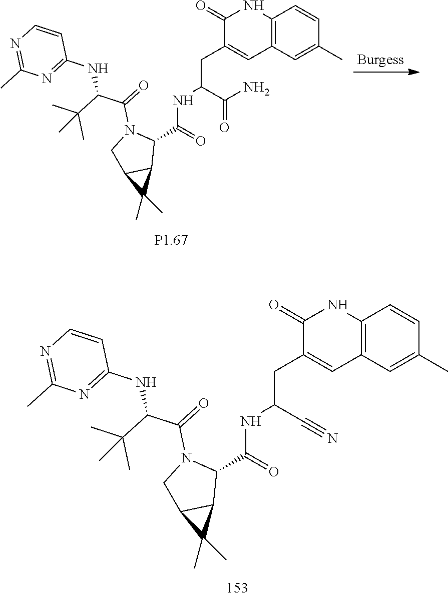

To a solution of P1.67 (100.0 mg, 0.2 mmol, 1.0 eq) in DCM (1.1 mL) was added methyl N-(triethylammoniumsulfonyl)carbamate (101.0 mg, 0.4 mmol, 2.5 eq) and then resulting mixture was stirred at rt for overnight. Then reaction mixture concentrated to get crude residue. The residue purified with C-18 reverse-phase column chromatography to give compound 153 (70.0 mg, 97% yield) as a white solid. ¹H NMR (400 MHz, MeOH), δ: 7.87-7.84 (m, 2H), 7.42-7.40 (m, 1H), 7.35-7.32 (m, 1H), 7.23-7.21 (m, 1H), 6.49-6.46 (m, 1H), 5.30-5.20 (m, 1H), 4.93-4.87 (m, 1H), 4.32-4.23 (m, 2H), 4.13-3.94 (m, 1H), 3.29-3.03 (m, 2H), 2.42-2.37 (m, 6H), 1.63-1.31 (m, 1H), 1.23-0.75 (m, 16H). LCMS (ESI) m/z calc. for C₃₂H₃₉N₇O₃ 569.70. found, 570.2 [M+H]⁺.

### Example 18. (1R,2S,5S)—N-(1-Cyano-2-(6-methyl-2-oxo-1,2-dihydroquinolin-3-yl)ethyl)-3-((S)-3,3-dimethyl-2-((5-methylthiazol-2-yl)amino)butanoyl)-6,6-dimethyl-3-azabicyclo[3.1.0]hexane-2-carboxamide (Compound 152)

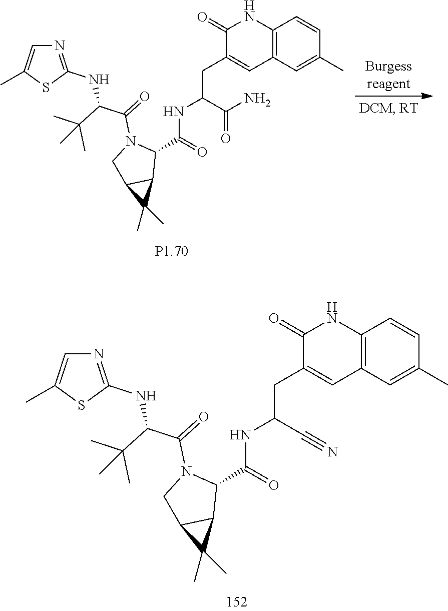

To a solution of P1.70 (70 mg, 0.118 mmol, 1.0 eq) in DCM (2.0 mL) was added Burgess reagent (70.3 mg, 0.295 mmol, 2.5 eq). The resulting mixture was stirred at rt for overnight. The reaction mixture was concentrated to get crude residue. The residue was purified by C-18 reversed-phase column chromatography (mobile phase A: water, mobile phase B: methanol, UV: 214 and 254 nm, Flow rate: 35 mL/min, Gradient: 70-80% (% B)) to give compound 152 (30 mg, 44% yield) as a white solid. ¹H NMR (400 MHz, CD₃OD), δ: 7.89 (s, 1H), 7.87 (s, 1H), 7.45-7.23 (m, 6H), 6.58 (s, 1H), 6.56 (s, 1H), 5.25-5.18 (m, 2H), 4.36 (s, 1H), 4.35 (s, 1H), 4.21-4.20 (m, 2H), 4.11-3.73 (m, 4H), 3.32-3.18 (m, 3H), 2.85-2.83 (m, 1H), 2.40 (s, 3H), 2.39 (s, 3H), 2.22 (s, 6H), 1.59-1.52 (m, 1H), 1.44-1.41 (m, 1H), 1.25-1.23 (m, 1H), 1.18-1.16 (m, 1H), 1.10-0.80 (s, 30H); LCMS (ESI): m/z 575.7 [M+H]⁺.

### Example 19. (1R,2S,5S)—N-(2-(6-Chloro-2-oxo-1,2-dihydroquinolin-3-yl)-1-cyanoethyl)-3-((S)-3,3-dimethyl-2-(2,2,2-trifluoroacetamido)butanoyl)-6,6-dimethyl-3-azabicyclo[3.1.0]hexane-2-carboxamide (Compound 104)

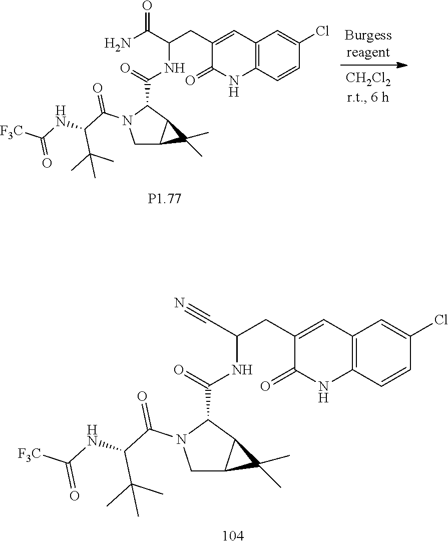 [SMILES: CC(C)(C)[C@H](NC(=O)C(F)(F)F)C(=O)N1C[C@H]2[C@@H]([C@H]1C(=O)NC(Cc1cc3cc(Cl)ccc3[nH]c1=O)C(N)=O)C2(C)C ; CC(C)(C)[C@H](NC(=O)C(F)(F)F)C(=O)N1C[C@H]2[C@@H]([C@H]1C(=O)NC(C#N)Cc1cc3cc(Cl)ccc3[nH]c1=O)C2(C)C]

To a solution of P1.77 (66.7 mg, 0.109 mmol, 1.0 eq) in DCM (0.80 mL) was added Burgess reagent (64.9 mg, 0.272 mmol, 2.5 eq), and the resulting mixture was stirred at rt for 6 h. The solution was treated with water and extracted with DCM for three times. The combined organic layers were washed with brine, dried over Na₂SO₄₍ₛ₎, filtered, and concentrated in vacuo to give the residue. The residue was purified by C-18 reversed-phase column chromatography (mobile phase A: water (with 0.1% NH₄HCO₃), mobile phase B: acetonitrile, UV: 214 and 254 nm, Flow rate: 35 mL/min, Gradient: 3-90% (% B)) to give 104 (48 mg, 74% yield) as a white solid. ¹H NMR (400 MHz, DMSO-d₆), δ: 12.08 (br. s, 2H), 9.40-9.38 (m, 1H), 8.99 (d, J=8.0 Hz, 1H), 8.87 (d, J=7.6 Hz, 1H), 7.88-7.86 (m, 2H), 7.77-7.76 (m, 2H), 7.56-7.49 (m, 2H), 7.32-7.30 (m, 2H), 5.18-5.07 (m, 2H), 4.38-4.35 (m, 2H), 4.15 (s, 1H), 4.13 (s, 1H), 3.99-3.90 (m, 1H), 3.85-3.77 (m, 1H), 3.64-3.61 (m, 2H), 3.14-2.92 (m, 4H), 1.56-1.49 (m, 1H), 1.34-1.28 (m, 1H), 1.07 (d, J=7.6 Hz, 1H), 1.02-0.78 (m, 31H); LCMS (ESI) m/z calc. for C₂₈H₃₁C₁F₃N₅O₄ 593.20. found, 594.2 and 596.2 [M+H]⁺; HPLC purity: 100%, t_{R}=22.311 min.

### Example 20. (1R,2S,5S)—N-(2-(7-Chloro-2-oxo-1,2-dihydroquinolin-3-yl)-1-cyanoethyl)-3-((S)-3,3-dimethyl-2-(2,2,2-trifluoroacetamido)butanoyl)-6,6-dimethyl-3-azabicyclo[3.1.0]hexane-2-carboxamide (Compound 105)

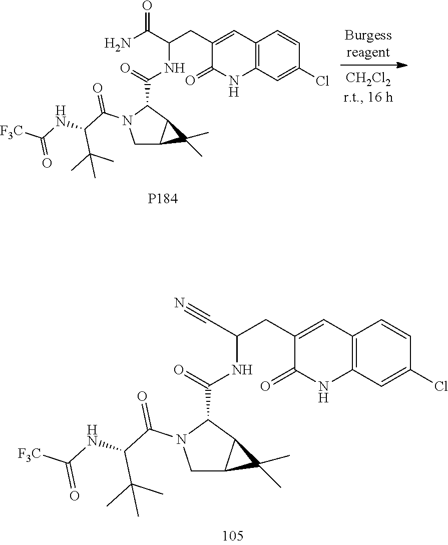 [SMILES: "P184" ; CC(C)(C)[C@H](NC(=O)C(F)(F)F)C(=O)N1C[C@H]2[C@@H]([C@H]1C(=O)NC(Cc1cc3ccc(Cl)cc3[nH]c1=O)C(N)=O)C2(C)C ; CC(C)(C)[C@H](NC(=O)C(F)(F)F)C(=O)N1C[C@H]2[C@@H]([C@H]1C(=O)NC(C#N)Cc1cc3ccc(Cl)cc3[nH]c1=O)C2(C)C]

To a solution of P1.84 (90.0 mg, 0.147 mmol, 1.0 eq) in DCM (1.0 mL) was added Burgess reagent (105.1 mg, 0.441 mmol, 3.0 eq), and the resulting mixture was stirred at rt for 16 h. The solution was treated with water and extracted with DCM for three times. The combined organic layers were washed with brine, dried over Na₂SO₄₍ₛ₎, filtered and concentrated in vacuo to give the residue. The residue was purified by C-18 reversed-phase column chromatography (mobile phase A: water (with 0.1% NH₄HCO₃), mobile phase B: acetonitrile, UV: 214 and 254 nm, Flow rate: 35 mL/min, Gradient: 3-90% (% B)) to give 105 (70 mg, 80% yield) as a white solid. ¹H NMR (400 MHz, DMSO-d₆), δ: 12.00 (br. s, 2H), 9.39-9.30 (m, 2H), 8.94 (d, J=8.0 Hz, 1H), 8.85 (d, J=7.6, Hz, 1H), 7.88-7.86 (m, 2H), 7.67-7.64 (m, 2H), 7.29-7.28 (m, 2H), 7.21-7.17 (m, 2H), 5.14-5.04 (m, 2H), 4.37-4.31 (m, 2H), 4.12 (s, 1H), 4.11 (s, 1H), 3.92-3.86 (m, 1H), 3.81-3.75 (m, 1H), 3.61-3.60 (m, 2H), 3.07-2.89 (m, 4H), 1.50-1.45 (m, 1H), 1.34-1.29 (m, 1H), 1.06 (d, J=8.0 Hz, 1H), 0.99-0.75 (m, 31H); LCMS (ESI) m/z calc. for C₂₈H₃₁C₁F₃N₅O₄ 593.20. found, 594.0 and 596.0 [M+H]⁺; HPLC purity: 98.8%, t_{R}=22.331 min.

### Example 21. (1R,2S,5S)—N-(1-Cyano-2-(1-methyl-2-oxo-1,2-dihydropyridin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 112)

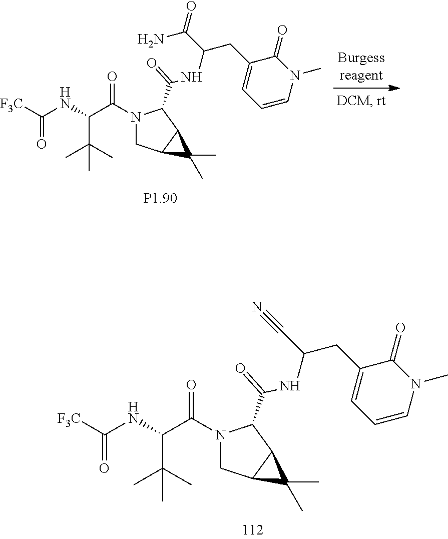 [SMILES: Cn1cccc(CC(NC(=O)[C@@H]2[C@@H]3[C@H](CN2C(=O)[C@@H](NC(=O)C(F)(F)F)C(C)(C)C)C3(C)C)C(N)=O)c1=O ; Cn1cccc(CC(C#N)NC(=O)[C@@H]2[C@@H]3[C@H](CN2C(=O)[C@@H](NC(=O)C(F)(F)F)C(C)(C)C)C3(C)C)c1=O]

To a solution of P1.90 (200 mg, 0.37 mmol) in DCM (4.0 mL) was added Burgess reagent (220 mg, 0.93 mmol), and the resulting mixture was stirred for overnight at rt. The mixture was concentrated under reduced pressure. The residue was purified by reverse-phase chromatography column chromatography (0% to 80% of MeOH in water) to give 112 (133 mg, 69%) as a white solid. ¹H NMR (400 MHz, CDCl₃), δ: 9.41-9.39 (m, 2H), 8.96-8.89 (m, 2H), 7.69-7.66 (m, 2H), 7.44-7.41 (m, 2H), 6.22-6.19 (m, 2H), 5.06-4.99 (m, 2H), 4.41-4.39 (m, 2H), 4.17 (s, 1H), 4.16 (s, 1H), 3.91-3.85 (m, 1H), 3.67-3.63 (m, 1H), 3.45 (s, 3H), 3.45 (s, 3H), 2.97-2.84 (m, 4H), 1.55-1.46 (m, 2H), 1.20 (d, J=7.6 Hz, 1H), 1.12 (d, J=8.0 Hz, 1H), 0.98-0.97 (m, 24H), 0.82-0.81 (d, J=2.0 Hz, 6H); LCMS (ESI) m/z calc. for C₂₅H₃₂F₃N₅O₄ 523.24. found, 524.7 [M+H]⁺. HPLC purity: 96.99%, t_{R}=19.359 min.

### Example 22. (1R,2S,5S)—N-(1-Cyano-2-(2-oxo-1,2,5,6,7,8-hexahydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 121)

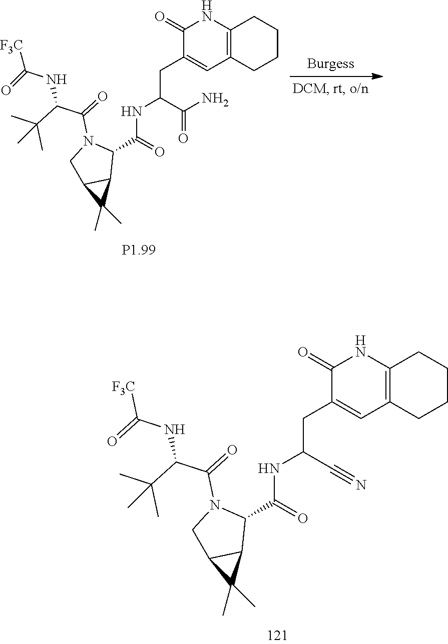 [SMILES: CC(C)(C)[C@H](NC(=O)C(F)(F)F)C(=O)N1C[C@H]2[C@@H]([C@H]1C(=O)NC(Cc1cc3c([nH]c1=O)CCCC3)C(N)=O)C2(C)C ; CC(C)(C)[C@H](NC(=O)C(F)(F)F)C(=O)N1C[C@H]2[C@@H]([C@H]1C(=O)NC(C#N)Cc1cc3c([nH]c1=O)CCCC3)C2(C)C]

A solution of P1.99 (46 mg, 0.08 mmol, 1.0 eq) and Burgess reagent (47 mg, 0.20 mmol, 2.5 eq) in DCM (1 mL) was stirred at rt for overnight. The mixture was concentrated and the residue was purified by C18 column (5-100% MeOH in H₂O) to give compound 121 (16 mg, 36%) as a white solid. ¹H NMR (400 MHz, CD₃OD), δ: 7.33 (s, 1H), 7.31 (s, 1H), 5.11-5.04 (m, 2H), 4.56-4.55 (m, 1H), 4.28-4.25 (m, 2H), 4.09-3.99 (m, 2H), 3.84-3.81 (m, 2H), 3.08-2.93 (m, 4H), 2.58-2.51 (m, 8H), 1.78-1.76 (m, 8H), 1.63-1.52 (m, 2H), 1.32-1.21 (m, 2H), 1.07-0.90 (m, 30H); LCMS (ESI) m/z calc. for C₂₈H₃₆F₃N₅O₄ 563.27. found, 564.2 [M+H]⁺; HPLC purity: 99.5%, t_{R}=20.77 min. Compound 121 contains two diastereoisomers.

### Example 23. (1R,2S,5S)—N-(1-Cyano-2-(2-oxo-1,2,5,6,7,8-hexahydroquinolin-3-yl)ethyl)-3-((S)-3,3-dimethyl-2-(methylsulfonamido)butanoyl)-6,6-dimethyl-3-azabicyclo[3.1.0]hexane-2-carboxamide (Compound 122)

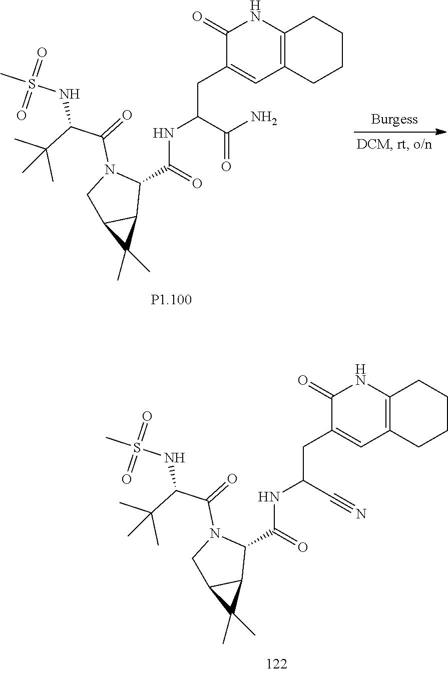 [SMILES: CC(C)(C)[C@H](NS(C)(=O)=O)C(=O)N1C[C@H]2[C@@H]([C@H]1C(=O)NC(Cc1cc3c([nH]c1=O)CCCC3)C(N)=O)C2(C)C ; CC(C)(C)[C@H](NS(C)(=O)=O)C(=O)N1C[C@H]2[C@@H]([C@H]1C(=O)NC(C#N)Cc1cc3c([nH]c1=O)CCCC3)C2(C)C]

To a solution of P1.100 (100 mg, 0.18 mmol, 1.0 eq) in DCM (2 mL) was added Burgess reagent (106 mg, 0.45 mmol, 2.5 eq), and the resulting mixture was stirred at rt for overnight. The mixture was concentrated and the residue was purified by C18 column (5-100% MeOH in H₂O) to give compound 122 (67 mg, 70%) as a white solid. ¹H NMR (400 MHz, CD₃OD), δ: 7.34 (s, 1H), 7.31 (s, 1H), 5.12-5.04 (m, 2H), 4.30 (s, 2H), 4.02-3.92 (m, 4H), 3.86-3.81 (m, 2H), 3.13-2.90 (m, 10H), 2.59-2.51 (m, 8H), 1.78-1.75 (m, 8H), 1.63-1.53 (m, 2H), 1.31-1.18 (m, 2H), 1.07-0.97 (m, 30H); LCMS (ESI) m/z calc. for C₂₇H₃₉N₅O₅S 545.27. found, 546.2 [M+H]⁺; HPLC purity: 98.2%, t_{R}=17.63 min.

### Example 24. (1R,2S,5S)—N-(1-Cyano-2-(2-oxo-1,5,7,8-tetrahydro-2H-pyrano[4,3-b]pyridine-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 123)

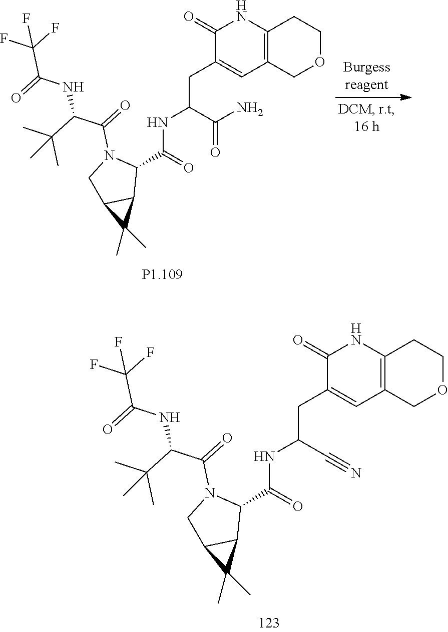 [SMILES: CC(C)(C)[C@H](NC(=O)C(F)(F)F)C(=O)N1C[C@H]2[C@@H]([C@H]1C(=O)NC(Cc1cc3c([nH]c1=O)CCOC3)C(N)=O)C2(C)C ; CC(C)(C)[C@H](NC(=O)C(F)(F)F)C(=O)N1C[C@H]2[C@@H]([C@H]1C(=O)NC(C#N)Cc1cc3c([nH]c1=O)CCOC3)C2(C)C]

To a solution of P1.109 (176 mg, 0.298 mmol, 1.0 eq) in DCM (4 mL) was added Burgess reagent (179 mg, 0.745 mmol, 2.5 eq) and the reaction solution was stirred at rt for 16 h. The reaction solution was concentrated under reduced pressure and the residue was purified by C18 flash column (5% to 100% methanol in H₂O) to afford compound 123 (44 mg, 26%) as a white solid. ¹H NMR (400 MHz, CD₃OD), δ: 7.27-7.26 (m, 2H), 5.11-5.06 (m, 2H), 4.56-4.46 (m, 6H), 4.27 (s, 2H), 4.06-3.81 (m, 8H), 3.11-2.94 (m, 4H), 2.67-2.63 (m, 4H), 1.62-1.53 (m, 2H), 1.33-1.22 (m, 2H), 1.08-0.88 (m, 30H); LCMS (ESI) m/z calc. for C₂₇H₃₄F₃N₅O₅ 565.25. found, 566.1 [M+H]⁺. HPLC purity: 100.00%, t_{R}=18.103 min.

### Example 25. (1R,2S,5S)—N-(1-Cyano-2-(2-oxo-1,5,7,8-tetrahydro-2H-pyrano[4,3-b]pyridine-3-yl)ethyl)-3-((S)-3,3-dimethyl-2-(methylsulfonamido)butanoyl)-6,6-dimethyl-3-azabicyclo[3.1.0]hexane-2-carboxamide (Compound 124)

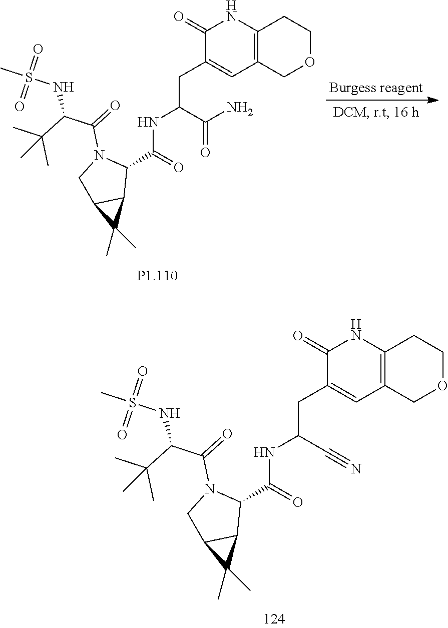 [SMILES: CC(C)(C)[C@H](NS(C)(=O)=O)C(=O)N1C[C@H]2[C@@H]([C@H]1C(=O)NC(Cc1cc3c([nH]c1=O)CCOC3)C(N)=O)C2(C)C ; CC(C)(C)[C@H](NS(C)(=O)=O)C(=O)N1C[C@H]2[C@@H]([C@H]1C(=O)NC(C#N)Cc1cc3c([nH]c1=O)CCOC3)C2(C)C]

To a solution of P1.110 (306 mg, 0.541 mmol, 1.0 eq) in DCM (5 mL) was added Burgess reagent (322 mg, 1.35 mmol, 2.5 eq) and the reaction solution was stirred at rt for 16 h. The reaction solution was concentrated under reduced pressure and the residue was purified by C18 flash column (5-100% methanol in H₂O) to afford compound 124 (36 mg, 12%) as a white solid. ¹H NMR (400 MHz, CD₃OD), δ: 7.28-7.27 (m, 2H), 5.11-5.05 (m, 2H), 4.57-4.48 (m, 4H), 4.28 (s, 2H), 4.00-3.82 (m, 10H), 3.09-2.90 (m, 10H), 2.67-2.64 (m, 4H), 1.63-1.53 (m, 2H), 1.32-1.19 (m, 2H), 1.08-0.95 (m, 30H); LCMS (ESI) m/z calc. for C₂₆H₃₇N₅O₆S 547.25. found, 548.1 [M+H]⁺. HPLC purity: 99.84%, t_{R}=14.799 min.

### Example 26. (1R,2S,5S)—N-(1-Cyano-2-(6,6-difluoro-2-oxo-1,2,5,6,7,8-hexahydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 125)

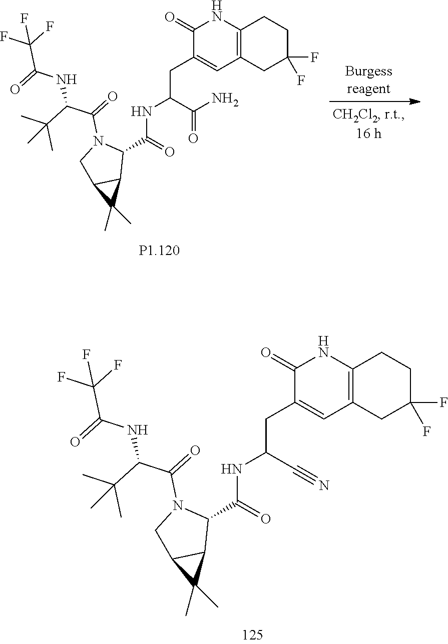 [SMILES: CC(C)(C)[C@H](NC(=O)C(F)(F)F)C(=O)N1C[C@H]2[C@@H]([C@H]1C(=O)NC(Cc1cc3c([nH]c1=O)CCC(F)(F)C3)C(N)=O)C2(C)C ; CC(C)(C)[C@H](NC(=O)C(F)(F)F)C(=O)N1C[C@H]2[C@@H]([C@H]1C(=O)NC(C#N)Cc1cc3c([nH]c1=O)CCC(F)(F)C3)C2(C)C]

A solution of P1.120 (0.10 g, 0.16 mmol) and Burgess reagent (0.116 g, 0.487 mmol) in DCM (1.6 mL) was stirred at room temperature for 16 h. The mixture was concentrated. The residue was purified by C18 column (5-100% MeOH in H₂O containing 1% NH₄HCO₃) to give compound 125 (35 mg, 36%) as a white solid. ¹H NMR (400 MHz, DMSO-d₆), δ: 11.80 (br. s, 2H), 9.48-9.30 (m, 2H), 9.07-8.86 (m, 2H), 7.28-7.16 (m, 2H), 5.13-4.92 (m, 2H), 4.43-4.34 (m, 2H), 4.18-4.12 (m, 2H), 3.99-3.85 (m, 2H), 3.71-3.58 (m, 2H), 3.03-2.77 (m, 8H), 2.75-2.67 (m, 4H), 2.27-2.10 (m, 4H), 1.60-1.41 (m, 2H), 1.22-1.12 (m, 2H), 1.03-0.93 (m, 24H), 0.87-0.77 (m, 6H); LCMS (ESI) m/z calc. for C₂₈H₃₄F₅N₅O₄ 599.25. found, 600.1 [M+H]⁺; HPLC purity: 100.0%, t_{R}=20.53 min.

### Example 27. (1R,2S,5S)—N-(1-Cyano-2-(6,6-difluoro-2-oxo-1,2,5,6,7,8-hexahydroquinolin-3-yl)ethyl)-3-((S)-3,3-dimethyl-2-(methylsulfonamido)butanoyl)-6,6-dimethyl-3-azabicyclo[3.1.0]hexane-2-carboxamide (Compound 126)

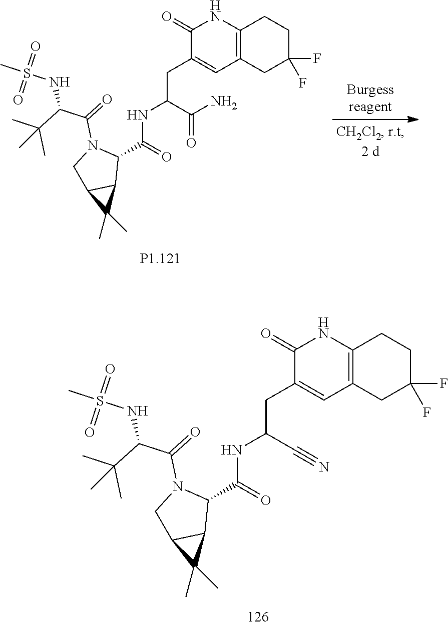 [SMILES: CC(C)(C)[C@H](NS(C)(=O)=O)C(=O)N1C[C@H]2[C@@H]([C@H]1C(=O)NC(Cc1cc3c([nH]c1=O)CCC(F)(F)C3)C(N)=O)C2(C)C ; CC(C)(C)[C@H](NS(C)(=O)=O)C(=O)N1C[C@H]2[C@@H]([C@H]1C(=O)NC(C#N)Cc1cc3c([nH]c1=O)CCC(F)(F)C3)C2(C)C]

To a solution of P1.121 (115.0 mg, 0.192 mmol, 1.0 eq) in DCM (1.9 mL) was added Burgess reagent (137.1 mg, 0.575 mmol, 3.0 eq), and the resulting mixture was stirred at rt for 2 days. The solution was treated with water and extracted with DCM three times. The combined organic layers were washed with brine, dried over Na₂SO₄₍ₛ₎, filtered, and concentrated in vacuo to give the residue, which was purified by C-18 reversed-phase column chromatography (mobile phase A: water, mobile phase B: acetonitrile, UV: 310 and 254 nm, Flow rate: 35 mL/min, Gradient: 3-90% (% B)) to give to give compound 126 (50 mg, 45% yield) as a white solid. ¹H NMR (400 MHz, DMSO-d₆), δ: 11.76 (s, 1H), 9.01-8.97 (m, 1H), 7.24 (s, 0.5H), 7.22 (s, 0.5H), 7.04 (d, J=9.6 Hz, 0.5H), 6.99 (d, J=9.2 Hz, 0.5H), 5.09-4.97 (m, 1H), 4.18 (d, J=4.0 Hz, 1H), 3.87-3.65 (m, 3H), 3.19 (s, 0.5H), 3.18 (s, 0.5H), 3.04-2.86 (m, 4H), 2.85-2.84 (m, 3H), 2.75-2.67 (m, 2H), 2.27-2.14 (m, 2H), 1.58-1.52 (m, 0.5H), 1.51-1.46 (m, 0.5H), 1.20 (d, J=7.6 Hz, 0.5H), 1.12 (d, J=7.6 Hz, 0.5H), 1.04-0.93 (m, 12H), 0.90 (d, J=3.2 Hz, 3H); LCMS (ESI) m/z calc. for C₂₇H₃₇F₂N₅O₅S 581.25. found, 582.1 [M+H]⁺.

### Example 28. (1R,2S,5S)—N-(1-Cyano-2-(6,6-dimethyl-2-oxo-1,2,5,6,7,8-hexahydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 127)

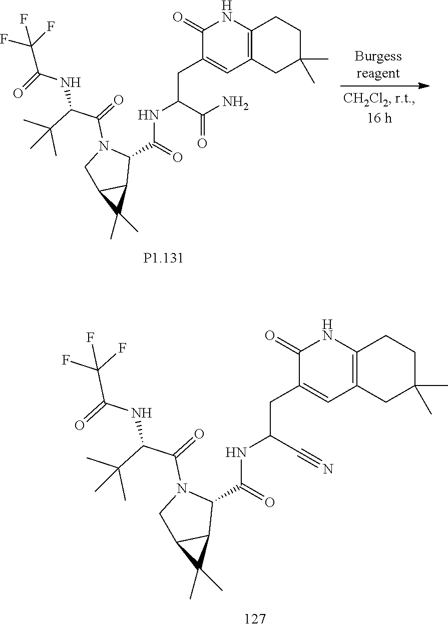 [SMILES: CC1(C)CCc2[nH]c(=O)c(CC(NC(=O)[C@@H]3[C@@H]4[C@H](CN3C(=O)[C@@H](NC(=O)C(F)(F)F)C(C)(C)C)C4(C)C)C(N)=O)cc2C1 ; CC1(C)CCc2[nH]c(=O)c(CC(C#N)NC(=O)[C@@H]3[C@@H]4[C@H](CN3C(=O)[C@@H](NC(=O)C(F)(F)F)C(C)(C)C)C4(C)C)cc2C1]

To a solution of P1.131 (118.0 mg, 0.194 mmol, 1.0 eq) in DCM (1.9 mL) was added Burgess reagent (138.4 mg, 0.581 mmol, 3.0 eq), and the resulting mixture was stirred at rt for 16 h. The solution was treated with water and extracted with DCM three times. The combined organic layers were washed with brine, dried over Na₂SO₄₍ₛ₎, filtered, and concentrated in vacuo to give the residue, which was purified by C-18 reversed-phase column chromatography (mobile phase A: water (with 0.1% NH₄HCO₃), mobile phase B: acetonitrile, UV: 310 and 254 nm, Flow rate: 35 mL/min, Gradient: 3-90% (% B)) to give compound 127 (23 mg, 20% yield) as a white solid. ¹H NMR (400 MHz, DMSO-d₆), δ: 11.64-11.62 (m, 1H), 9.45-9.40 (m, 1H), 9.06 (d, J=8.0 Hz, 0.5H), 8.97 (d, J=6.8 Hz, 0.5H), 7.16 (s, 0.5H), 7.14 (s, 1H), 5.09-4.94 (m, 1H), 4.41-4.39 (m, 1H), 4.15 (s, 1H), 4.02-3.87 (m, 1H), 3.69-3.60 (m, 1H), 2.91-2.74 (m, 2H), 2.49-2.43 (m, 2H), 2.25-2.12 (m, 2H), 1.57-1.41 (m, 3H), 1.22-1.10 (m, 1H), 1.03-0.82 (m, 21H); LCMS (ESI) m/z calc. for C₃₀H₄₀F₃N₅O₄ 591.30. found, 592.2 [M+H]⁺.

### Example 29. (1R,2S,5S)—N-(1-Cyano-2-(6,6-dimethyl-2-oxo-1,2,5,6,7,8-hexahydroquinolin-3-yl)ethyl)-3-((S)-3,3-dimethyl-2-(methylsulfonamido)butanoyl)-6,6-dimethyl-3-azabicyclo[3.1.0]hexane-2-carboxamide (Compound 128)

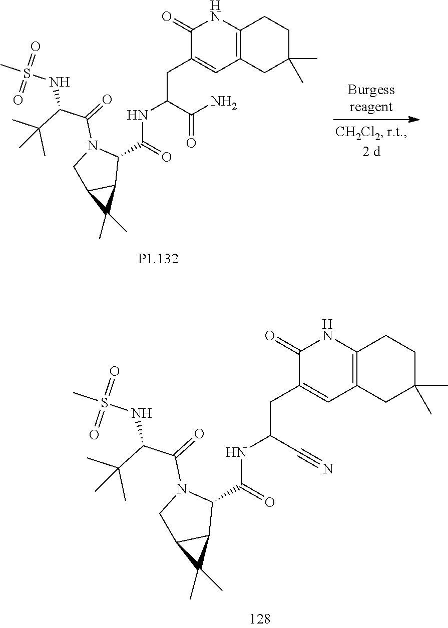

To a solution of P1.132 (109.0 mg, 0.184 mmol, 1.0 eq) in DCM (1.8 mL) was added Burgess reagent (131.7 mg, 0.552 mmol, 3.0 eq), and the resulting mixture was stirred at rt for 2 days. The solution was treated with water and extracted with DCM three times. The combined organic layers were washed with brine, dried over Na₂SO₄₍ₛ₎, filtered, and concentrated in vacuo to give the residue, which was purified by C-18 reversed-phase column chromatography (mobile phase A: water, mobile phase B: acetonitrile, UV: 310 and 254 nm, Flow rate: 35 mL/min, Gradient: 3-90% (% B)) to give compound 128 (73 mg, 69% yield) as a white solid. ¹H NMR (400 MHz, DMSO-d₆), δ: 11.61 (br. s, 0.5H), 11.56 (br. s, 0.5H), 9.03-8.99 (m, 1H), 7.16 (s, 0.5H), 7.16 (s, 0.5H), 7.03-6.97 (m, 1H), 5.10-4.94 (m, 1H), 4.18-4.17 (s, 1H), 3.89-3.76 (m, 2H), 3.71-3.68 (m, 1H), 2.92-2.75 (m, 5H), 2.48-2.43 (m, 2H), 2.23-2.13 (m, 2H), 1.58-1.41 (m, 3H), 1.19 (d, J=8.0 Hz, 0.5H), 1.08 (d, J=7.6 Hz, 0.5H), 1.03-0.94 (m, 12H), 0.93-0.88 (m, 9H); LCMS (ESI) m/z calc. for C₂₉H₄₃N₅O₅S 573.30. found, 574.2 [M+H]⁺.

### Example 30. (1R,2S,5S)—N-(1-Cyano-2-(6-methyl-2-oxo-1,2-dihydroquinolin-3-yl)ethyl)-3-((S)-3,3-dimethyl-2-((4-methylthiazol-2-yl)amino)butanoyl)-6,6-dimethyl-3-azabicyclo[3.1.0]hexane-2-carboxamide (Compound 151)

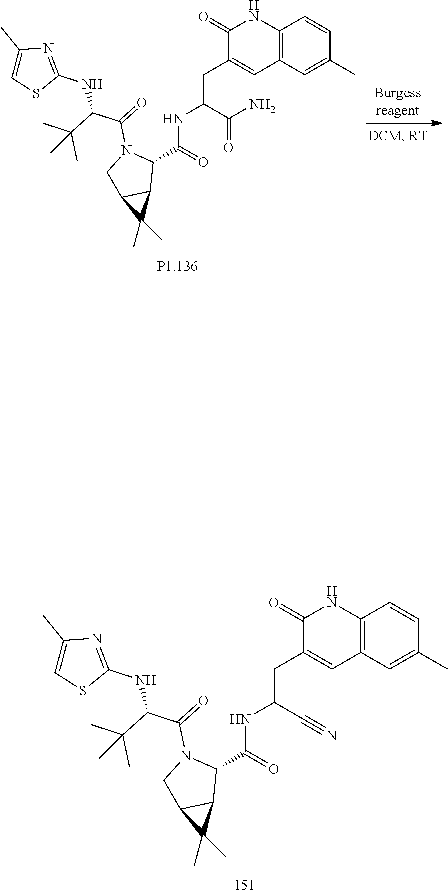

To a solution of P1.136 (68 mg, 0.115 mmol, 1.0 eq) in DCM (2.0 mL) was added methyl N-(triethylammoniumsulfonyl)carbamate (95.9 mg, 0.405 mmol, 3.5 eq) then resulting mixture stirred at rt for 2 days. Then reaction mixture concentrated to get crude residue. The residue purified with C-18 reversed-phase column chromatography (mobile phase A: water, mobile phase B: methanol, UV: 214 and 254 nm, Flow rate: 35 mL/min, Gradient: 70-80% (% B)) to give compound 151 (35 mg, 52% yield) as a white solid. ¹H NMR (400 MHz, CD₃OD), δ: 7.87 (s, 0.5H), 7.86 (s, 0.5H), 7.42 (s, 1H), 7.45-7.42 (m, 1H), 7.22 (d, J=8.4 Hz, 1H), 6.06 (m, 1H), 5.18-5.28 (m, 1H), 4.60 (s, 0.5H), 4.40 (s, 0.5H), 4.43 (s, 0.5H), 4.30-3.88 (m, 3H), 3.26-3.06 (m, 2H), 2.39-2.38 (m, 3H), 2.13-2.12 (m, 3H), 1.58-1.56 (m, 0.5H), 1.32-1.28 (m, 1H), 1.19 (d, J=7.6 Hz, 0.5H), 1.06-0.79 (m, 15H).

### Example 31. (1R,2S,5S)—N—((S)-1-Cyano-2-(6-methyl-2-oxo-1,2-dihydroquinolin-3-yl)ethyl)-3-((S)-3,3-dimethyl-2-((5-methylthiazol-2-yl)amino)butanoyl)-6,6-dimethyl-3-azabicyclo[3.1.0]hexane-2-carboxamide (Compound 158)

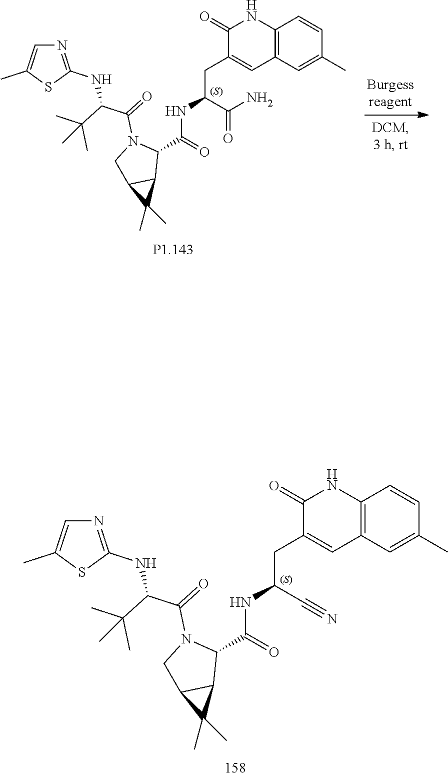

To a solution of P1.143 (50 mg, 0.0844 mmol) in DCM (2.0 mL) was added methyl N-(triethylammoniumsulfonyl)carbamate (60.3 mg, 0.253 mmol). The resulting mixture was stirred at rt for 3 h. The reaction mixture was extracted with DCM, combined organic layers dried over Na₂SO₄₍ₛ₎, filtered, and concentrated to obtain a crude residue which was purified silica gel column chromatography by using 0-20% methanol in DCM to obtain target product 158 as a white solid (20 mg, 42% yield). ¹H NMR (400 MHz, CDCl₃), δ: 7.87 (s, 1H), 7.44 (s, 1H), 7.35 (d, J=8.4 Hz 1H), 7.22 (d, J=8.4 Hz 1H), 6.56 (s, 1H), 5.18-5.14 (m, 1H), 4.34 (s, 1H), 4. 19 (s, 1H), 4.10 (d, J=10 Hz, 1H) 4.01-3.97 (m, 1H), 3.18-3.15 (m, 2H), 2.38 (s, 3H), 2.39 (s, 3H), 1.56-1.52 (m, 1H), 1.15 (d, 7.6 Hz, 1H), 0.96 (s, 9H), 0.93 (s, 3H), 0.80 (s, 3H); LCMS (ESI): 575.3 [M+H]⁺.

### Example 32. (1R,2S,5S)—N—((S)-1-Cyano-2-(6-methyl-2-oxo-1,2-dihydroquinolin-3-yl)ethyl)-3-((S)-3,3-dimethyl-2-((2-methylpyrimidin-4-yl)amino)butanoyl)-6,6-dimethyl-3-azabicyclo[3.1.0]hexane-2-carboxamide (Compound 159)

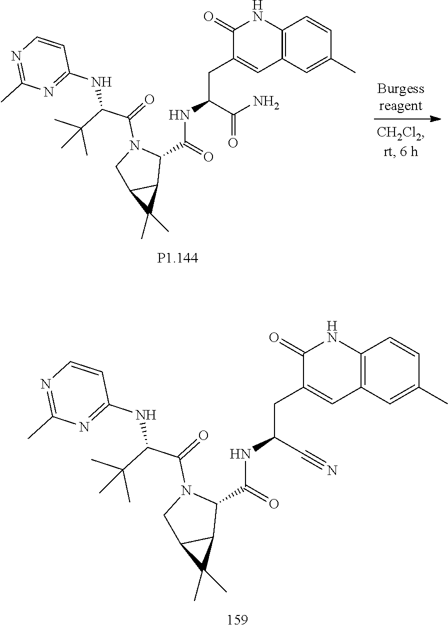

To a solution of P1.144 (109 mg, 0.185 mmol) in DCM (3.0 mL) was added methyl N-(triethylammoniumsulfonyl)carbamate (132.2 mg, 0.354 mmol). The resulting mixture was stirred at rt for 6 h. Then reaction mixture was diluted with water and extracted by DCM. The combined organic layers were dried over Na₂SO₄ (s), filtered, and concentrated to obtain a residue. The residue was purified by silica gel column chromatography (0-10% dichloromethane in methanol) to give desired compound 159 (63 mg, 61% yield) as a white solid. ¹H NMR (400 MHz, MeOD-d₄): 7.89-7.86 (m, 2H), 7.46 (s, 1H), 7.38-7.35 (m, 1H), 7.23 (d, J=8.4 Hz, 1H), 6.46 (d, J=6.0 Hz, 1H), 5.22-5.18 (m, 1H), 4.93 (br. s, 1H), 4.34-4.30 (m, 1H), 4.23 (s, 1H), 4.15-4.10 (m, 1H), 3.21-3.16 (m, 2H), 2.42 (s, 3H), 2.40 (s, 3H), 1.62-1.59 (m, 1H), 1.21 (d, J=7.6 Hz, 1H)), 1.00 (s, 9H), 0.98 (s, 3H), 0.77 (s, 3H). LRMS (ESI) m/z 570.2 [M+H]⁺.

### Example 33. (1R,2S,5S)—N—((S)-1-Cyano-2-(6-methyl-2-oxo-1,2-dihydroquinolin-3-yl)ethyl)-3-((S)-3,3-dimethyl-2-((4-methylpyrimidin-2-yl)amino)butanoyl)-6,6-dimethyl-3-azabicyclo[3.1.0]hexane-2-carboxamide (Compound 160)

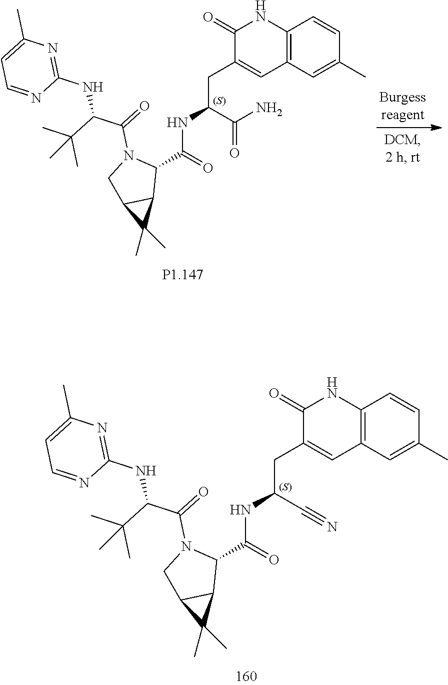

To a solution of P1.147 (170 mg, 0.289 mmol) in DCM (3.0 mL) was added methyl N-(triethylammoniumsulfonyl)carbamate (207 mg, 0.867 mmol). After the resulting mixture was stirred at rt for 2 h, the reaction mixture was treated with water and extracted with DCM. The combined organic layers were dried over Na₂SO₄₍ₛ₎, filtered and concentrated to give a residue. The residue was purified by silica gel column chromatography (0-20% methanol in DCM) to obtain target product 160 as a white solid (100 mg, 61% yield). ¹H NMR (400 MHz, CDCl₃), δ: 10.63 (br. s, 1H), 9.33 (br. s, 1H), 7.94 (d, J=5.2 Hz 1H), 7.26 (s, 1H), 7.03 (s, 1H), 6.85 (d, J=8.4 Hz, 1H), 6.61 (d, J=8.4 Hz, 1H), 6.29 (d, J=4.4 Hz, 1H), 5.34-5.30 (m, 2H), 4.63 (br. s, 1H), 4.59 (s, 1H), 4.28-4.18 (m, 2H), 3.60-3.53 (m, 1H), 2.95-2.90 (m, 1H), 2.28 (s, 3H), 2.15 (br. s, 1H), 1.67-1.65 (m, 1H), 1.55 (d, J=7.2 Hz 1H), 1.26 (s, 9H), 1.06 (s, 3H), 0.84 (s, 3H); LRMS(ESI) m/z 570.4 [M+H]⁺.

### Example 34. (1R,2S,5S)—N—((S)-1-Cyano-2-(6-methyl-2-oxo-1,2-dihydroquinolin-3-yl)ethyl)-3-((S)-3,3-dimethyl-2-((2-(trifluoromethyl)pyrimidin-4-yl)amino)butanoyl)-6,6-dimethyl-3-azabicyclo[3.1.0]hexane-2-carboxamide (Compound 161)

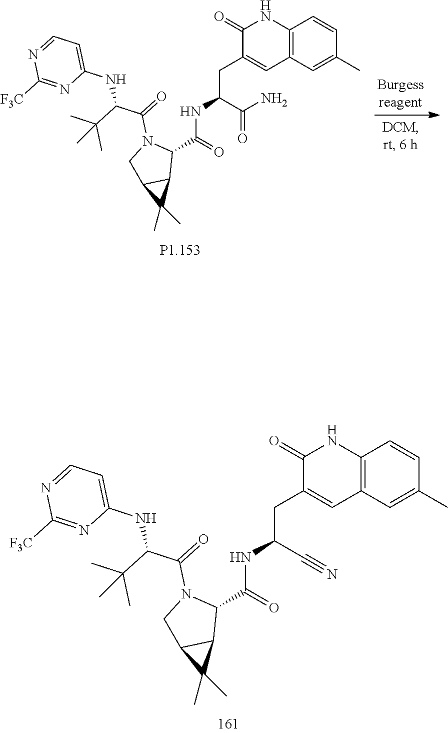 [SMILES: Cc1ccc2[nH]c(=O)c(C[C@H](NC(=O)[C@@H]3[C@@H]4[C@H](CN3C(=O)[C@@H](Nc3ccnc(C(F)(F)F)n3)C(C)(C)C)C4(C)C)C(N)=O)cc2c1 ; Cc1ccc2[nH]c(=O)c(C[C@@H](C#N)NC(=O)[C@@H]3[C@@H]4[C@H](CN3C(=O)[C@@H](Nc3ccnc(C(F)(F)F)n3)C(C)(C)C)C4(C)C)cc2c1]

To a solution of P1.153 (65 mg, 0.101 mmol) in DCM (3.0 mL) was added methyl N-(triethylammoniumsulfonyl)carbamate (85 mg, 0.354 mmol). The resulting mixture was stirred at rt for 6 h. The solution was treated with water and extracted with DCM. The combined organic layers were dried over Na₂SO₄ (s), filtered and concentrated to obtain a residue. The residue was purified by silica gel column chromatography (0-10% DCM in methanol) to give desired product 161 (38 mg, 60% yield) as a white solid. ¹H NMR (400 MHz, CD₃OD), δ: 8.12 (d, J=6.0 Hz 1H), 7.88 (s, 1H), 7.44 (s, 1H), 7.37-7.34 (m, 1H), 7.23 (d, J=8.4 Hz, 1H), 6.79 (d, J=6.0 Hz, 1H), 5.21 (dd, J=8.0, 6.8 Hz, 1H), 5.01 (s, 1H), 4.24-4.09 (m, 3H), 3.26-3.15 (m, 2H), 2.39 (s, 3H), 1.61-1.58 (m, 1H), 1.20 (d, J=7.6 Hz 1H)), 1.02 (s, 9H), 0.94 (s, 3H), 0.69 (s, 3H); LCMS (ESI) m/z 624.1 [M+H]⁺.

### Example 35. (2S)—N-((2S)-1-((1-Cyano-2-(6-methyl-2-oxo-1,2-dihydroquinolin-3-yl)ethyl)amino)-1-oxo-3-phenylpropan-2-yl)-3,3-dimethyl-2-((2,2,2-trifluoroethyl)sulfonamido)butanamide (Compound 164)

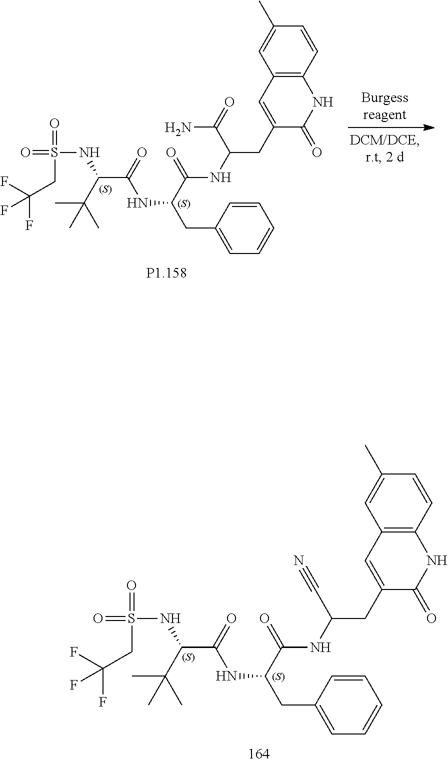

To a solution of P1.158 (93 mg, 0.142 mmol, 1.0 eq) in DCM (2 mL)/dimethoxyethane (10 mL) was added Burgess reagent (85.0 mg, 0.356 mmol, 2.5 eq) and the reaction solution was stirred at rt for 16 h. Added the eq of Burgess reagent every 4 h after started material was consumed. The reaction solution was concentrated under reduced pressure and the residue was purified by C18 flash column (5% to 100% methanol in H₂O) to afford compound 164 (27.9 mg, 31%) as a white solid. ¹H NMR (400 MHz, CD₃OD), δ: 7.82-7.78 (m, 2H), 7.42-7.34 (m, 4H), 7.24-6.97 (m, 10H), 5.17-5.09 (m, 2H), 4.59-4.53 (m, 2H), 3.78-3.51 (m, 6H), 3.16-2.71 (m, 8H), 2.38 (s, 6H), 0.92 (s, 9H), 0.75 (s, 9H).; LCMS (ESI) m/z calc. for C₃₀H₃₄F₃N₅O₅S 633.2. found, 634.1 [M+H]⁺. HPLC purity: 91.35%, t_{R}=20.842 min.

### Example 36. (2S)—N-((2S)-1-((1-Cyano-2-(6-methyl-2-oxo-1,2-dihydroquinolin-3-yl)ethyl)amino)-1-oxo-3-(2,4,5-trifluorophenyl)propan-2-yl)-3,3-dimethyl-2-((2,2,2-trifluoroethyl)sulfonamido)butanamide (Compound 166)

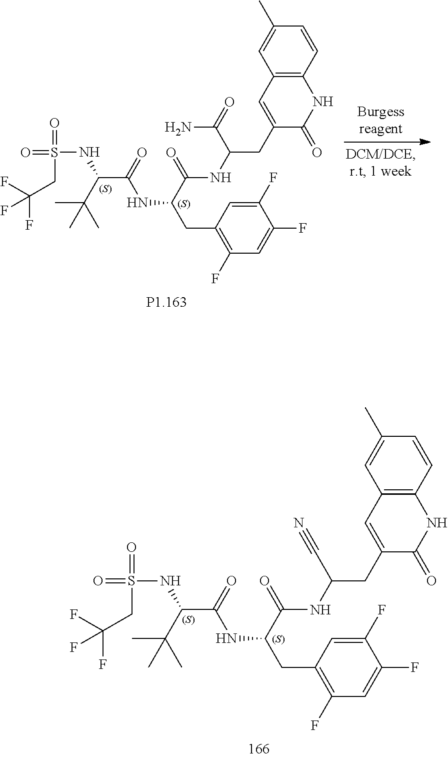

To a solution of P1.163 (40.0 mg, 0.057 mmol, 1.0 eq) in DCM (1.5 mL) and 1,2-dichloroethane (1.5 mL) was added Burgess reagent (40.5 mg, 0.170 mmol, 3.0 eq), and the resulting mixture was stirred at rt for 16 h. The reaction was added extra amount of Burgess reagent until the starting material was consumed totally. The solution was treated with water and extracted with DCM three times. The combined organic layers were washed with brine, dried over Na₂SO₄₍ₛ₎, filtered, and concentrated in vacuo to give the residue. The residue was purified by C-18 reversed-phase column chromatography (mobile phase A: water, mobile phase B: acetonitrile, UV: 214 and 254 nm, Flow rate: 35 mL/min, Gradient: 3-90% (% B)) to give compound 166 (21 mg, 54% yield) as a white solid. ¹H NMR (400 MHz, DMSO-d₆), δ: 11.86 (s, 1H), 11.85 (s, 1H), 9.02 (d, J=8.0 Hz, 1H), 8.96 (d, J=7.2 Hz, 1H), 8.39-8.38 (m, 2H), 7.85-7.77 (m, 4H), 7.44-7.04 (m, 12H), 5.15-5.03 (m, 2H), 4.59-4.56 (m, 2H), 4.04-3.65 (m, 6H), 3.04-2.55 (m, 8H), 2.33 (s, 3H), 2.32 (s, 3H), 0.86 (s, 9H), 0.73 (s, 9H).; LCMS (ESI) m/z calc. for C₃₀H₃₁F₆N₅O₅S 687.20. found, 688.1 [M+H]⁺.

### Example 37. (1R,2S,5S)—N—((S)-1-Cyano-2-(6-methyl-2-oxo-1,2-dihydroquinolin-3-yl)ethyl)-3-((S)-3,3-dimethyl-2-(methylsulfonamido)butanoyl)-N,6,6-trimethyl-3-azabicyclo[3.1.0]hexane-2-carboxamide (Compound 169)

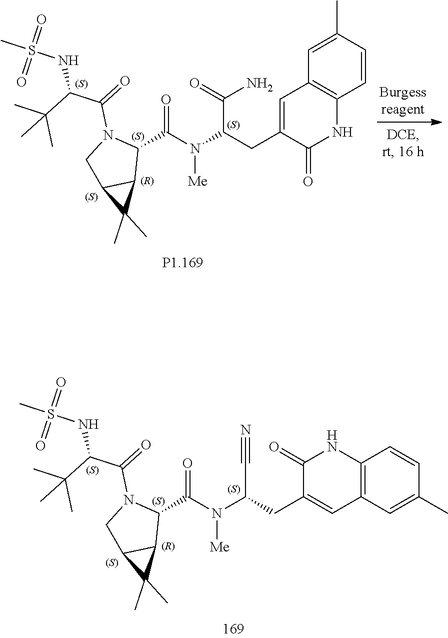

To a solution of P1.169 (75 mg, 0.128 mmol) in DCM (16 mL) was added Burgess reagent (91 mg, 0.383 mmol). After the mixture was stirred at rt for 16 h, the mixture was diluted with EtOAc and washed with NaHCO_{3(aq)} and brine. The organic layer was dried over MgSO₄, filtered, and concentrated under reduced pressure. The residue was purified by silica gel chromatography (0-5% MeOH in DCM with 1% NH₄OH_{(aq)}) and then preparative HPLC (10-100% MeOH in H₂O) to give compound 169 (17 mg, 21%) as a white solid. ¹H NMR (400 MHz, CD3OD): δ 7.93-7.81 (m, 1H), 7.48-7.41 (m, 1H), 7.40-7.31 (m, 1H), 7.25-7.20 (m, 1H), 6.07-5.69 (m, 1H), 4.66-4.58 (m, 1H), 3.98-3.67 (m, 3H), 3.29-3.09 (m, 5H), 2.87 (s, 3H), 2.41-2.36 (m, 3H), 1.40-1.06 (m, 2H), 1.04-0.90 (m, 13H), 0.78 (s, 2H); LCMS (ESI): 570.1 [M+H]⁺; HPLC purity: 66.7%, t_{R}=19.18 min; 33.3%, t_{R}=19.56 min. Note: ZE83-0347 is a mixture of two diastereoisomers. The ratio is 2 to 1 which is determined by HPLC.

### Example 38. tert-Butyl ((2S)-1-((1R,2S,5S)-2-((1-cyano-2-(6-methyl-2-oxo-1,2-dihydroquinolin-3-yl)ethyl)carbamoyl)-6,6-dimethyl-3-azabicyclo[3.1.0]hexan-3-yl)-3,3-dimethyl-1-oxobutan-2-yl)carbamate (Compound 171)

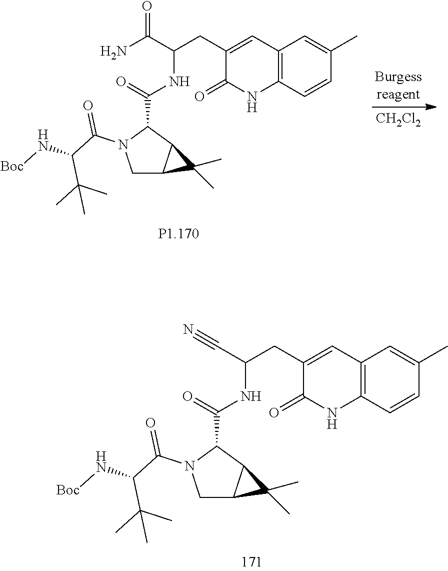 [SMILES: Cc1ccc2[nH]c(=O)c(CC(NC(=O)[C@@H]3[C@@H]4[C@H](CN3C(=O)[C@@H](NC(=O)OC(C)(C)C)C(C)(C)C)C4(C)C)C(N)=O)cc2c1 ; "CH2Cl2" ; Cc1ccc2[nH]c(=O)c(CC(C#N)NC(=O)[C@@H]3[C@@H]4[C@H](CN3C(=O)[C@@H](NC(=O)OC(C)(C)C)C(C)(C)C)C4(C)C)cc2c1]

To a solution of P1.170 (70 mg, 0.12 mmol) in DCM (1.5 mL) was added Burgess reagent (70 mg, 0.29 mmol, 2.5 eq), and the resulting mixture was stirred for overnight. The mixture was concentrated and the residue was purified by C18 column (5-100% MeOH in H₂O) to give compound 171 (38 mg, 56%) as a white solid. ¹H NMR (400 MHz, CD₃OD), δ: 7.89-7.87 (m, 1H), 7.46-7.45 (m, 1H), 7.38-7.36 (m, 1H), 7.25-7.23 (m, 1H), 5.27-5.17 (m, 1H), 4.24-4.14 (m, 2H), 4.03-3.83 (m, 2H), 3.30-3.03 (m, 2H), 2.40-2.39 (m, 3H), 1.57-1.27 (m, 11H), 0.98-0.87 (m, 15H); LCMS (ESI) m/z calc. for C₃₂H₄₃N₅O₅ 577.33. found, 478.2 [M−100+H]⁺; HPLC purity: 100%, t_{R}=23.37 min.

### Example 39. (1R,2S,5S)—N—((R)-1-Cyano-2-(2-oxo-1,2-dihydro-1,6-naphthyridin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 173)

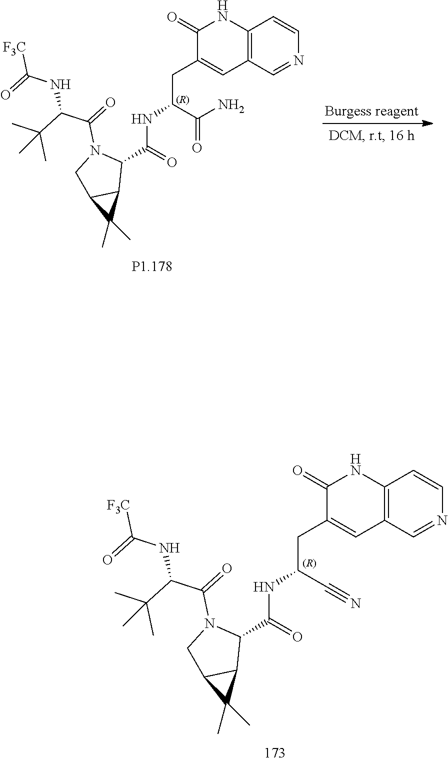

To a solution of P1.178 (56.0 mg, 0.0967 mmol, 1.0 eq) in DCM (2.0 mL) was added Burgess reagent (57.6 mg, 0.242 mmol, 2.5 eq) and the reaction solution was stirred at rt for 16 h. The reaction solution was concentrated under reduced pressure. The residue was purified by C18 flash column chromatography (5-100% methanol in H₂O) to afford compound 173 (39 mg, 72%) as a white solid. ¹H NMR (400 MHz, CD₃OD), δ: 8.82 (s, 1H), 8.44 (d, J=6.0 Hz, 1H), 8.03 (s, 1H), 7.27 (d, J=6.0 Hz, 1H), 5.23-5.19 (m, 1H), 4.53 (s, 1H), 4.25 (s, 1H), 3.93-3.78 (m, 2H), 3.29-3.10 (m, 2H), 1.40-1.37 (m, 1H), 1.12-1.05 (m, 9H), 0.98-0.87 (m, 7H); LCMS (ESI) m/z calc. for C₂₇H₃₁F₃N₆O₄ 560.24. found, 561.6 [M+H]⁺. HPLC purity: 96.99%, t_{R}=17.291 min.

### Example 40. (1R,2S,5S)—N—((S)-1-Cyano-2-(2-oxo-1,2-dihydro-1,6-naphthyridin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 174)

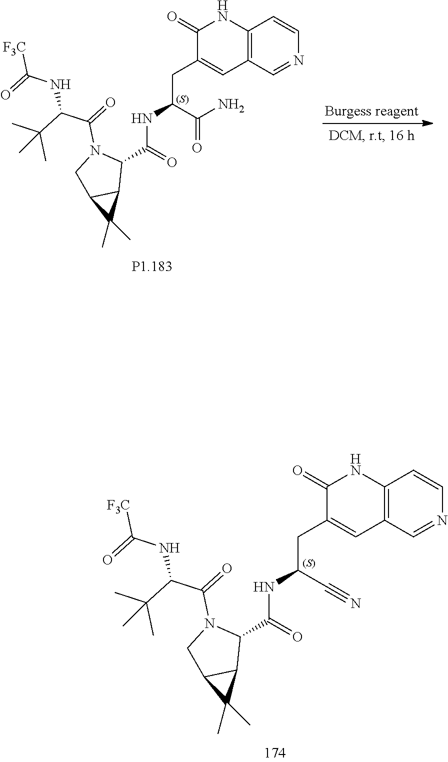

To a solution of P1.183 (59.0 mg, 0.102 mmol, 1.0 eq) in DCM (2.0 mL) was added Burgess reagent (60.7 mg, 0.255 mmol, 2.5 eq) and the reaction solution was stirred at rt for 16 h. The reaction solution was concentrated under reduced pressure and the residue was purified by C18 flash column chromatography (5-100% methanol in H₂O) to afford compound 174 (37.5 mg, 65%) as a white solid. ¹H NMR (400 MHz, CD₃OD), δ: 8.83 (s, 1H), 8.43 (d, J=6.0 Hz, 1H), 8.02 (s, 1H), 7.26 (d, J=6.0 Hz, 1H), 5.29-5.25 (m, 1H), 4.48 (s, 1H), 4.25 (s, 1H), 3.99-3.96 (m, 1H), 3.82-3.80 (m, 1H), 3.21-3.17 (m, 2H), 1.59-1.56 (m, 1H), 1.30-1.28 (m, 1H), 1.06-1.02 (m, 3H), 0.90-0.85 (m, 12H); LCMS (ESI) m/z calc. for C₂₇H₃₁F₃N₆O₄ 560.24. found, 561.1 [M+H]⁺. HPLC purity: 100%, t_{R}=17.225 min.

### Example 41. (1R,2S,5S)—N—((S)-1-Cyano-2-(2-oxo-1,2-dihydro-1,6-naphthyridin-3-yl)ethyl)-3-((S)-3,3-dimethyl-2-(methylsulfonamido)butanoyl)-6,6-dimethyl-3-azabicyclo[3.1.0]hexane-2-carboxamide (Compound 188)

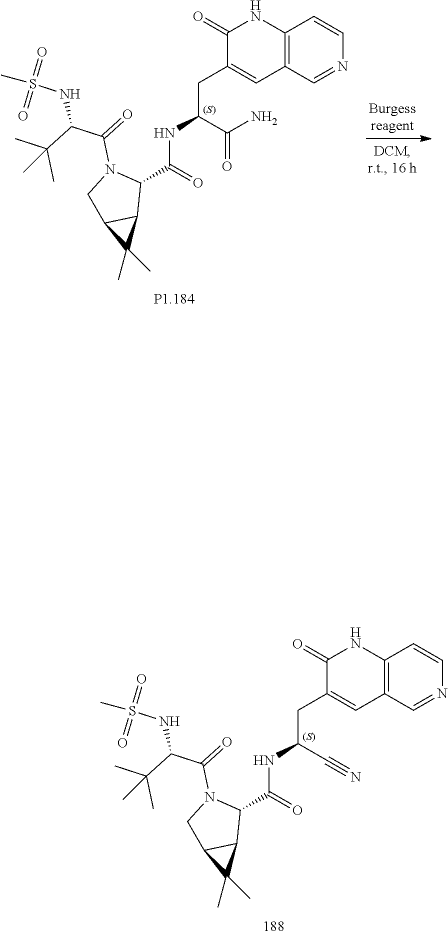

To a solution of P1.184 (TFA salt, 56.0 mg, 0.0998 mmol, 1.0 eq) in DCM (2.0 mL) was added Burgess reagent (59.5 mg, 0.249 mmol, 2.5 eq) and triethylamine (14 μL, 0.0998 mmol, 1.0 eq). The reaction solution was stirred at rt for 16 h. The reaction solution was concentrated under reduced pressure and the residue was purified by C18 flash column chromatography (5-100% methanol in H₂O) to afford 188 (16.2 mg, 30%) as a white solid. ¹H NMR (400 MHz, CD₃OD): δ 8.85 (s, 1H), 8.42 (d, J=6.0 Hz, 1H), 8.03 (s, 1H), 7.25 (d, J=6.0 Hz, 1H), 5.27-5.23 (m, 1H), 4.28 (s, 1H), 3.96-3.80 (m, 3H), 3.21-3.17 (m, 2H), 2.88 (s, 3H), 1.60-1.57 (m, 1H), 1.30-1.28 (m, 1H), 1.05-0.90 (m, 15H); LCMS (ESI) m/z calc. for C₂₆H₃₄N₆O₅S 542.23. found, 543.2 [M+H]⁺. HPLC purity: 96.85%, t_{R}=13.797 min.

### Example 42. (1R,2S,5S)—N—((R)-1-Cyano-2-(2-oxo-1,2-dihydro-1,6-naphthyridin-3-yl)ethyl)-3-((S)-3,3-dimethyl-2-(methylsulfonamido)butanoyl)-6,6-dimethyl-3-azabicyclo[3.1.0]hexane-2-carboxamide (Compound 189)

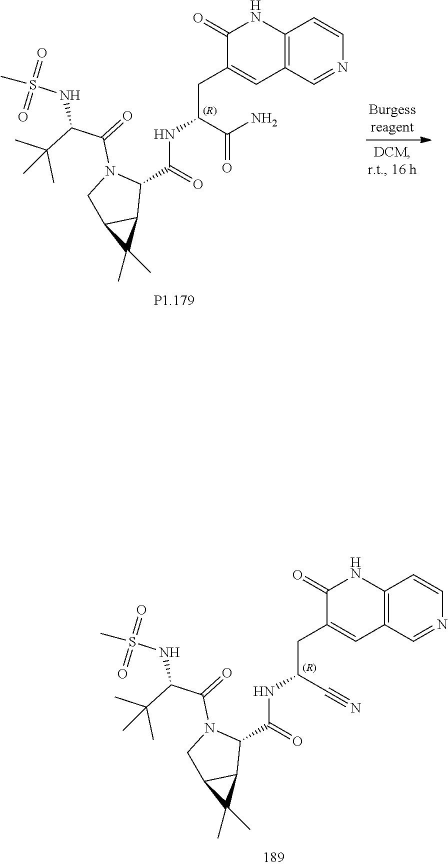

To a solution of P1.179 (93.0 mg, 0.165 mmol, 1.0 eq) in DCM (2.0 mL) was added Burgess reagent (98.7 mg, 0.414 mmol, 2.5 eq) and the reaction solution was stirred at rt for 16 h. The reaction solution was concentrated under reduced pressure and the residue was purified by C18 flash column (5% to 100% methanol in H₂O) to afford 189 (55 mg, 61%) as a yellow solid. ¹H NMR (400 MHz, CD₃OD), δ: 8.83 (s, 1H), 8.43 (d, J=6.0 Hz, 1H), 8.03 (s, 1H), 7.26 (d, J=6.0 Hz, 1H), 5.22-5.18 (m, 1H), 4.27 (s, 1H), 3.91-3.77 (m, 3H), 3.28-3.11 (m, 2H), 2.89 (s, 3H), 1.41-1.38 (m, 1H), 1.09-0.95 (m, 10H), 0.98-0.92 (m, 6H); LCMS (ESI) m/z calc. for C₂₆H₃₄N₆O₅S 542.23. found, 543.1 [M+H]⁺. HPLC purity: 93.92%, t_{R}=13.614 min.

### Example 43. (1R,2S,5S)—N—((S)-1-Cyano-2-(6-methyl-2-oxo-1,2-dihydroquinolin-3-yl)ethyl)-6,6-dimethyl-3-(2-(3-methyl-1H-pyrazol-5-yl)acetyl)-3-azabicyclo[3.1.0]hexane-2-carboxamide (Compound 190)

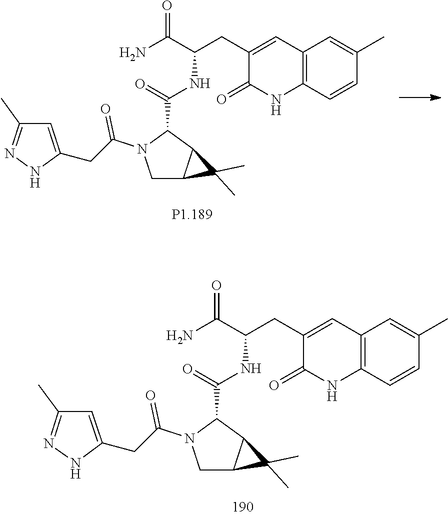

To a solution of P1.189 (24 mg, 0.048 mmol) in pyridine (1.0 mL) was added POCl₃ (0.020 mL, 0.22 mmol) at 0° C. After the mixture was stirred at rt for 16 h, MeOH was added at rt. The mixture was concentrated. The residue was purified by C18 reverse phase column (5-100% methanol in H₂O) to give a desired product 190 (8 mg, 35% yield). ¹H NMR (400 MHz, CD₃OD), δ: 7.90-7.68 (m, 1H), 7.48-7.15 (m, 3H), 5.95-5.65 (m, 1H), 5.43-5.15 (m, 1H), 4.38-4.12 (m, 1H), 3.96-3.52 (m, 4H), 3.29-3.07 (m, 2H), 2.40-2.28 (m, 3H), 2.24-2.16 (m, 3H), 1.40-1.31 (m, 1H), 1.24-1.02 (m, 1H), 0.96-0.69 (m, 6H); ¹H NMR (400 MHz, CDCl₃), δ: 7.84-7.65 (m, 1H), 7.36-7.26 (m, 1H), 7.25-7.00 (m, 2H), 5.88-5.68 (m, 1H), 5.16-5.00 (m, 1H), 4.51-4.14 (m, 1H), 3.88-3.77 (m, 1H), 3.76-3.63 (m, 1H), 3.54-3.38 (m, 2H), 3.35-3.14 (m, 2H), 2.43-2.33 (m, 3H), 2.24-2.04 (m, 3H), 1.59-1.53 (m, 1H), 1.41-1.24 (m, 1H), 1.02-0.71 (m, 6H); LCMS (ESI) m/z calc. for C₂₇H₃₀N₆O₃ 486.24. found, 487.3 [M+H]⁺; HPLC purity: 91.45%, t_{R}=16.292 min.

### Example 44. (1R,2S,5S)—N—((S)-1-Cyano-2-(6-methyl-2-oxo-1,2-dihydroquinolin-3-yl)ethyl)-6,6-dimethyl-3-(2-(3-(trifluoromethyl)-1H-pyrazol-5-yl)acetyl)-3-azabicyclo[3.1.0]hexane-2-carboxamide (Compound 191)

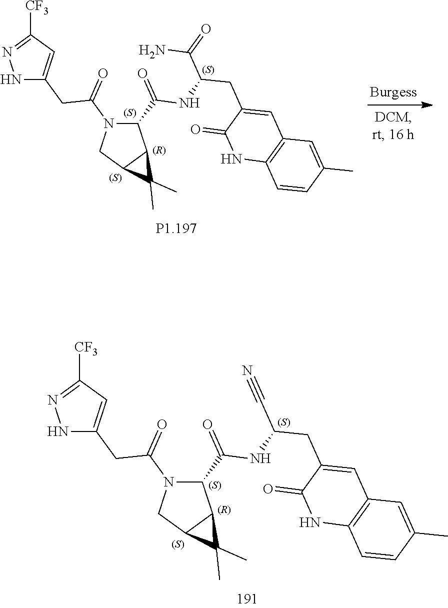

To a solution of P1.197 (141 mg, 0.253 mmol) in DCM (2.5 mL) was added Burgess reagent (181 mg, 0.758 mmol). After the mixture was stirred at rt for 16 h, the mixture was diluted with EtOAc and washed with NaHCO_{3(aq)} and brine. The organic layer was dried over MgSO₄, filtered, and concentrated under reduced pressure. The residue was purified by C18 reverse phase chromatography (0-100% MeOH in H₂O) to give 191 (55 mg, 40%) as a white solid. ¹H NMR (400 MHz, DMSO-d₆), δ: 13.47-13.20 (m, 1H), 11.93-11.76 (m, 1H), 9.15-8.83 (m, 1H), 7.81-7.73 (m, 1H), 7.46-7.09 (m, 3H), 6.52-6.22 (m, 1H), 5.28-5.05 (m, 1H), 4.28-4.01 (m, 1H), 3.91-3.42 (m, 4H), 3.18-2.94 (m, 2H), 2.35-2.17 (m, 3H), 1.49-1.26 (m, 1H), 1.13-0.94 (m, 1H), 0.91-0.73 (m, 6H); LCMS (ESI) m/z calc. for C₂₇H₂₇F₃N₆O₃ 540.21. found, 541.1 [M+H]⁺; HPLC purity: 99.8%, t_{R}=19.158 min.

### Example 45. (1R,2S,5S)—N—((S)-1-Cyano-2-(6-methyl-2-oxo-1,2-dihydroquinolin-3-yl)ethyl)-6,6-dimethyl-3-(2-(3-methylisoxazol-5-yl)acetyl)-3-azabicyclo[3.1.0]hexane-2-carboxamide (Compound 192)

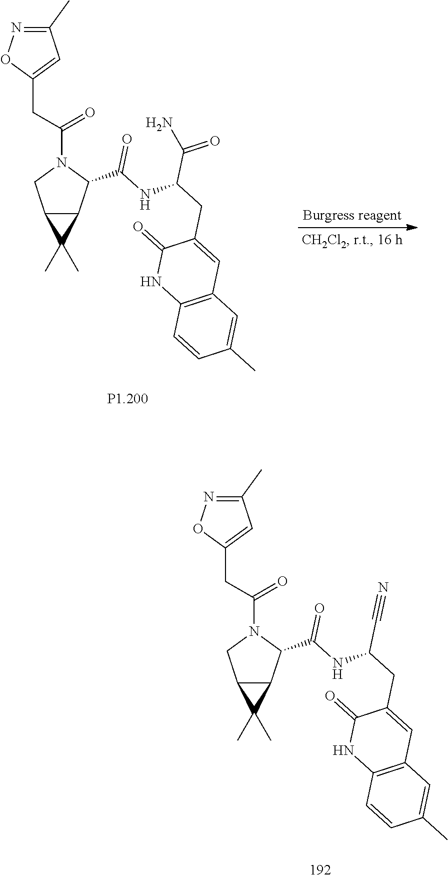 [SMILES: Cc1ccc2[nH]c(=O)c(C[C@H](NC(=O)[C@@H]3[C@@H]4[C@H](CN3C(=O)Cc3cc(C)no3)C4(C)C)C(N)=O)cc2c1 ; Cc1ccc2[nH]c(=O)c(C[C@@H](C#N)NC(=O)[C@@H]3[C@@H]4[C@H](CN3C(=O)Cc3cc(C)no3)C4(C)C)cc2c1]

To a solution of P1.200 (144.0 mg, 0.285 mmol, 1.0 eq) in DCM (2.9 mL) was added Burgess reagent (203.6 mg, 0.854 mmol, 3.0 eq), and the resulting mixture was stirred at rt for 16 h. The solution was treated with water and extracted with DCM three times. The combined organic layers were washed with brine, dried over Na₂SO₄₍ₛ₎, filtered, and concentrated in vacuo to give the residue. The residue was purified by C-18 reversed-phase column chromatography (mobile phase A: water, mobile phase B: acetonitrile, UV: 214 and 254 nm, Flow rate: 40 mL/min, Gradient: 3-90% (% B)) to give compound 192 (103 mg, 74% yield) as a white solid. ¹H NMR (400 MHz, CD₃OD), δ: 7.88-7.84 (m, 1H), 7.46 (s, 1H), 7.39-7.29 (m, 1H), 7.27-7.15 (m, 1H), 6.10-5.88 (m, 1H), 5.39-5.15 (m, 1H), 4.30-4.21 (m, 1H), 4.01-3.97 (m, 1H), 3.93-3.81 (m, 2H), 3.69-3.55 (m, 1H), 3.26-3.19 (m, 1H), 3.15-3.07 (m, 1H), 2.39-2.34 (m, 3H), 2.20-2.18 (m, 3H), 1.52-1.38 (m, 1H), 1.30-1.16 (m, 1H), 0.96-0.93 (m, 3H), 0.90-0.87 (m, 3H); LCMS (ESI) m/z calc. for C₂₇H₂₉N₅O₄ 487.22. found, 488.1 [M+H]⁺.

### Example 46. (1R,2S,5S)—N—((S)-1-Cyano-2-(6-methyl-2-oxo-1,2-dihydroquinolin-3-yl)ethyl)-6,6-dimethyl-3-(2-(3-(trifluoromethyl)isoxazol-5-yl)acetyl)-3-azabicyclo[3.1.0]hexane-2-carboxamide (Compound 193)

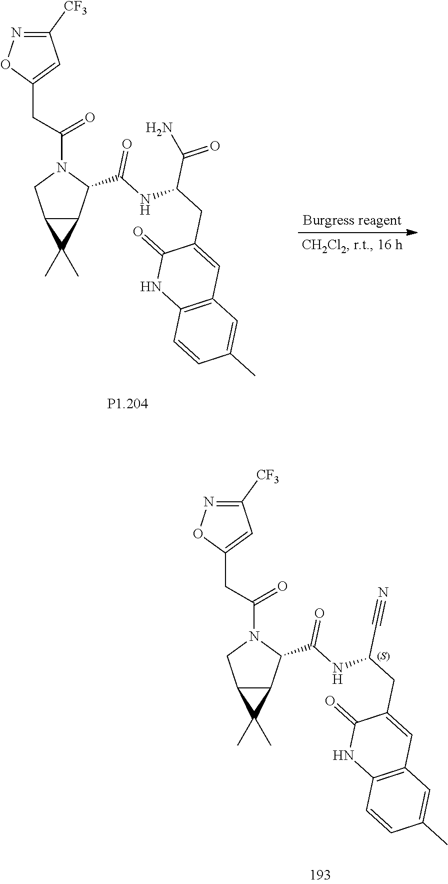

To a solution of P1.204 (93.0 mg, 0.166 mmol, 1.0 eq) in DCM (2.0 mL) was added Burgess reagent (118.8 mg, 0.499 mmol, 3.0 eq), and the resulting mixture was stirred at rt for 16 h. The solution was treated with water and extracted with DCM three times. The combined organic layers were washed with brine, dried over Na₂SO₄₍ₛ₎, filtered, and concentrated in vacuo to give the residue. The residue was purified by C-18 reversed-phase column chromatography (mobile phase A: water, mobile phase B: acetonitrile, UV: 214 and 254 nm, Flow rate: 40 mL/min, Gradient: 3-90% (% B)) to give compound 193 (63 mg, 70% yield) as a white solid. ¹H NMR (400 MHz, CDCl₃), δ: 10.19-10.03 (m, 1H), 9.40-9.18 (m, 1H), 7.83 (s, 0.1H), 7.43-7.31 (m, 1H), 7.12-7.07 (m, 1H), 6.87 (d, J=8.4 Hz, 1H), 6.53 (s, 1H), 6.37-6.25 (m, 1H), 5.24-4.97 (m, 1H), 4.55 (s, 1H), 4.24-3.93 (m, 2H), 3.92-3.72 (m, 1H), 3.64-3.56 (m, 1H), 3.50-3.33 (m, 1H), 3.16-3.02 (m, 1H), 2.40-2.31 (m, 3H), 1.62-1.23 (m, 2H), 1.08-1.04 (m, 3H), 0.93-0.88 (m, 3H); LCMS (ESI) m/z calc. for C₂₇H₂₆F₃N₅O₄ 541.19. found, 542.1 [M+H]⁺.

### Example 47. (1R,2S,5S)—N—((S)-1-Cyano-2-(6-methyl-2-oxo-1,2-dihydroquinolin-3-yl)ethyl)-6,6-dimethyl-3-(2-(3-(trifluoromethyl)isoxazol-5-yl)acetyl)-3-azabicyclo[3.1.0]hexane-2-carboxamide (Compound 194)

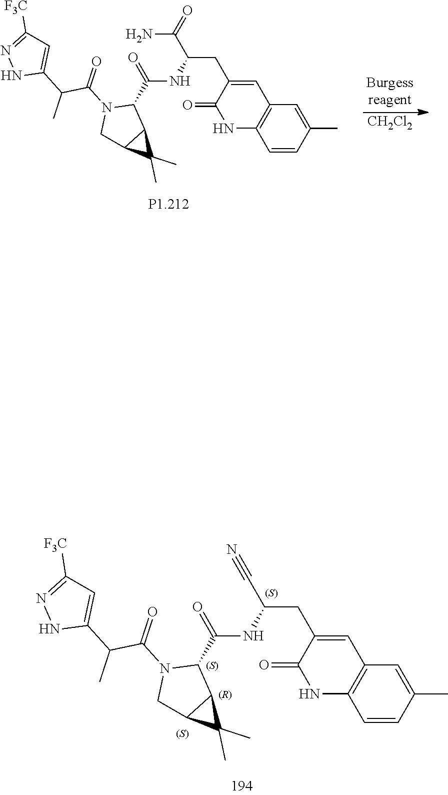

To a solution of P1.212 (70 mg, 0.122 mmol, 1.0 eq) in DCM (1.5 mL) was added Burgess reagent (72 mg, 0.301 mmol, 2.5 eq). The resulting mixture stirred at rt for 16 h. The reaction mixture was concentrated to get crude residue. The residue was purified by C-18 reversed-phase column chromatography (mobile phase A: water, mobile phase B: methanol, UV: 214 and 254 nm, Flow rate: 35 mL/min, Gradient: 70-80% (% B)) to give compound 194 (25 mg, 37% yield) as a white solid. ¹H NMR (400 MHz, CD₃OD), δ: 7.91-7.86 (m, 1H), 7.46-7.19 (m, 3H), 6.41-6.30 (m, 1H), 5.40-5.21 (m, 1H), 4.28-3.98 (m, 2H), 3.67-3.43 (m, 2H), 3.22-3.00 (m, 2H), 2.39-2.34 (m, 3H), 1.60-1.13 (m, 5H), 0.97-0.56 (m, 6H); LCMS (ESI) m/z found, 555.0 [M+H]⁺.

### Example 48. (1R,2S,5S)—N-[(1S)-1-Cyano-2-(7-methoxy-6-methyl-2-oxo-1H-quinolin-3-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 175)

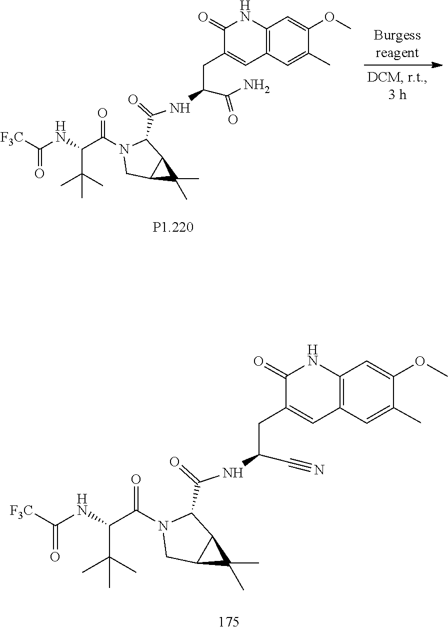 [SMILES: COc1cc2[nH]c(=O)c(C[C@H](NC(=O)[C@@H]3[C@@H]4[C@H](CN3C(=O)[C@@H](NC(=O)C(F)(F)F)C(C)(C)C)C4(C)C)C(N)=O)cc2cc1C ; COc1cc2[nH]c(=O)c(C[C@@H](C#N)NC(=O)[C@@H]3[C@@H]4[C@H](CN3C(=O)[C@@H](NC(=O)C(F)(F)F)C(C)(C)C)C4(C)C)cc2cc1C]

To a solution of P1.220 (159 mg, 0.256 mmol) in DCM (4.0 mL) was added methyl N-(triethylammoniumsulfonyl)carbamate (183 mg, 0.768 mmol). The resulting mixture stirred at rt for 3 h. The reaction mixture was treated with water and extracted with DCM. The combined organic layers dried over Na₂SO₄ (s), filtered, and concentrated to obtain a crude residue. The crude was purified by silica gel column chromatography (0-20% dichloromethane in methanol) to give desired product 175 as a white solid (138 mg, 89% yield). ¹H NMR (400 MHz, DMSO-d₆), δ: 11.79 (br. s, 1H), 9.42 (d, J=8.4 Hz, 1H), 8.86 (d, J=7.2 Hz, 1H), 7.73 (s, 1H), 7.39 (s, 1H), 6.80 (s, 1H), 5.08-5.01 (m, 1H), 4.40 (d, J=8.4 Hz, 1H), 4.13 (s, 1H), 3.97-3.93 (m, 1H), 3.81 (s, 3H), 3.63-3.61 (m, 1H), 3.00-2.98 (m, 2H), 2.14 (s, 3H), 1.54-1.50 (m, 1H), 1.08 (d, J=7.6 Hz, 1H)), 0.96-0.79 (m, 15H); LCMS (ESI): 604.1 [M+H]⁺.

### Example 49. (1R,2S,5S)—N—((S)-1-Cyano-2-(7-fluoro-6-methyl-2-oxo-1,2-dihydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 176)

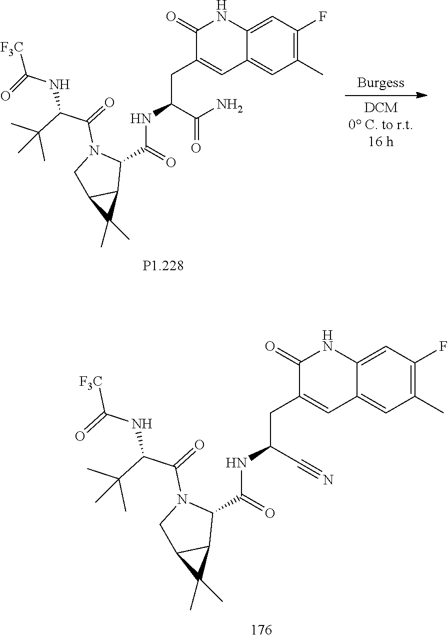 [SMILES: Cc1cc2cc(C[C@H](NC(=O)[C@@H]3[C@@H]4[C@H](CN3C(=O)[C@@H](NC(=O)C(F)(F)F)C(C)(C)C)C4(C)C)C(N)=O)c(=O)[nH]c2cc1F ; Cc1cc2cc(C[C@@H](C#N)NC(=O)[C@@H]3[C@@H]4[C@H](CN3C(=O)[C@@H](NC(=O)C(F)(F)F)C(C)(C)C)C4(C)C)c(=O)[nH]c2cc1F]

To a solution of P1.228 (165 mg, 0.27 mmol, 1.0 eq) in DCM (2.0 mL) was added Burgess reagent (161 mg, 0.68 mmol, 2.5 eq). The resulting mixture was stirred at rt for 16 h. The mixture was concentrated in vacuo to give the residue. The residue was purified by C-18 reversed-phase column chromatography (0-10% methanol in water) to give compound 176 (144 mg, 89% yield) as a white solid. ¹H NMR (400 MHz, CD₃OD), δ: 7.88 (s, 1H), 7.55 (d, J=8.0 Hz, 1H), 7.01 (d, J=10.8 Hz, 1H), 5.20 (t, J=7.4 Hz, 1H), 4.51 (s, 1H), 4.25 (s, 1H), 4.04-4.00 (m, 1H), 3.81 (d, J=10.4 Hz, 1H), 3.16 (d, J=7.5 Hz, 2H), 2.31 (s, 3H), 1.62-1.54 (m, 1H), 1.25 (d, J=7.6 Hz, 1H), 0.99-0.95 (m, 11H), 0.88 (s, 4H); LCMS (ESI) m/z calc. for C₂₉H₃₃F₄N₅O₄ 591.25. found, 592.1 [M+H]⁺; HPLC purity: 99%, t_{R}=22.342 min.

### Example 50. (1R,2S,5S)—N—((S)-1-Cyano-2-(6,7-difluoro-2-oxo-1,2-dihydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 177)

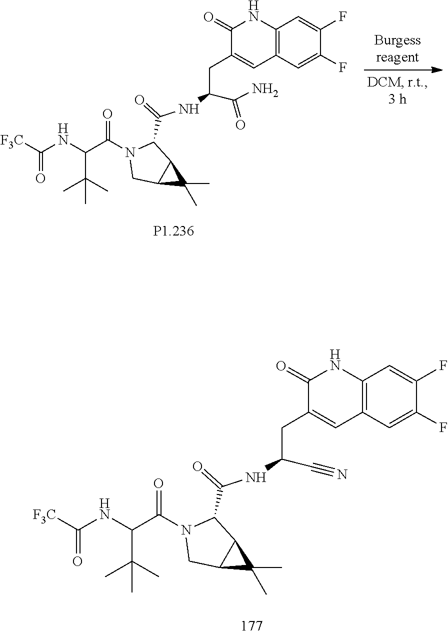 [SMILES: CC(C)(C)C(NC(=O)C(F)(F)F)C(=O)N1C[C@H]2[C@@H]([C@H]1C(=O)N[C@@H](Cc1cc3cc(F)c(F)cc3[nH]c1=O)C(N)=O)C2(C)C ; CC(C)(C)C(NC(=O)C(F)(F)F)C(=O)N1C[C@H]2[C@@H]([C@H]1C(=O)N[C@H](C#N)Cc1cc3cc(F)c(F)cc3[nH]c1=O)C2(C)C]

To a solution of P1.236 (282 mg, 0.459 mmol, 1.0 eq) in DCM (5.0 mL) was added Burgess reagent (328.1 mg, 1.377 mmol). The resulting mixture was stirred at rt for overnight. The reaction mixture was quenched by water and extracted with DCM. The combined organic layers were dried over Na₂SO₄ (s), filtered and concentrated to obtain crude residue. It was purified by silica gel column chromatography (0-20% DCM in methanol) to give desired product 177 as a light-yellow solid (138 mg, 50% yield). ¹H NMR (400 MHz, CDCl₃), δ: 10.94 (br. s, 1H), 8.76 (d, J=6.8 Hz, 1H), 7.37 (s, 1H), 7.17-7.13 (m, 1H), 6.66-6.62 (m, 2H), 5.36-5.32 (m, 1H), 5.50 (d, J=8.4 Hz, 1H), 4.46 (s, 1H), 4.13-4.09 (m, 1H), 3.91 (d, J=10.4 Hz, 1H), 3.02-2.98 (m, 1H), 1.68-1.65 (m, 1H), 1.56 (d, J=7.6 Hz, 1H)), 1.20 (s, 9H), 1.08 (s, 3H), 0.86 (s, 3H).

### Example 51. (1R,2S,5S)—N—((S)-1-Cyano-2-(4,6-dimethyl-2-oxo-1,2-dihydroquinolin-3-yl)-ethyl)-3-((S)-3,3-dimethyl-2-(2,2,2-trifluoroacetamido)butanoyl)-6,6-dimethyl-3-azabicyclo[3.1.0]hexane-2-carboxamide (Compound 178)

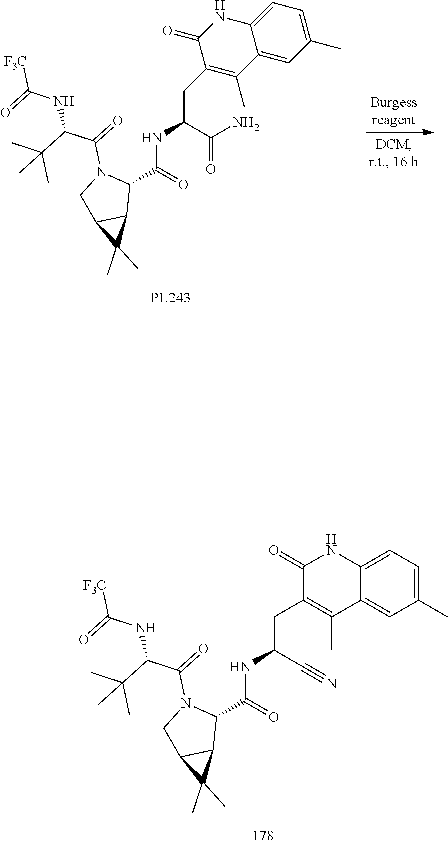 [SMILES: Cc1ccc2[nH]c(=O)c(C[C@H](NC(=O)[C@@H]3[C@@H]4[C@H](CN3C(=O)[C@@H](NC(=O)C(F)(F)F)C(C)(C)C)C4(C)C)C(N)=O)c(C)c2c1 ; Cc1ccc2[nH]c(=O)c(C[C@@H](C#N)NC(=O)[C@@H]3[C@@H]4[C@H](CN3C(=O)[C@@H](NC(=O)C(F)(F)F)C(C)(C)C)C4(C)C)c(C)c2c1]

To a solution of P1.243 (312 mg, 0.515 mmol, 1.0 eq) in DCM (5 mL) was added Burgess reagent (307 mg, 1.28 mmol, 2.5 eq) and the reaction solution was stirred at rt for 16 h. The reaction solution was concentrated under reduced pressure to give a residue. The residue was treated with methanol. The resulting precipitate was collected and washed with methanol to afford compound 178 (280 mg, 92%) as a white solid. ¹H NMR (400 MHz, DMSO-d₆), δ: 11.78 (br. s, 1H), 9.44 (d, J=8.4 Hz, 1H), 8.85 (d, J=7.6 Hz, 1H), 7.58 (s, 1H), 7.31 (d, J=8.4 Hz, 1H), 7.20 (d, J=8.4 Hz, 1H), 5.03 (q, J=7.6 Hz, 1H), 4.38 (d, J=8.4 Hz, 1H), 4.13 (s, 1H), 3.94-3.90 (m, 1H), 3.61-3.59 (m, 1H), 3.23-3.09 (m, 2H), 2.50 (s, 3H), 2.35 (s, 3H), 1.51-1.48 (m, 1H), 1.18-1.14 (m, 1H), 0.92-0.78 (m, 15H); LCMS (ESI) m/z calc. for C₃₀H₃₆F₃N₅O₄ 587.27. found, 588.1 [M+H]⁺; HPLC purity: 100.0%, t_{R}=22.491 min.

### Example 52. (1R,2S,5S)—N—((R)-1-Cyano-2-(2-oxo-1,2-dihydro-1,6-naphthyridin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 179)

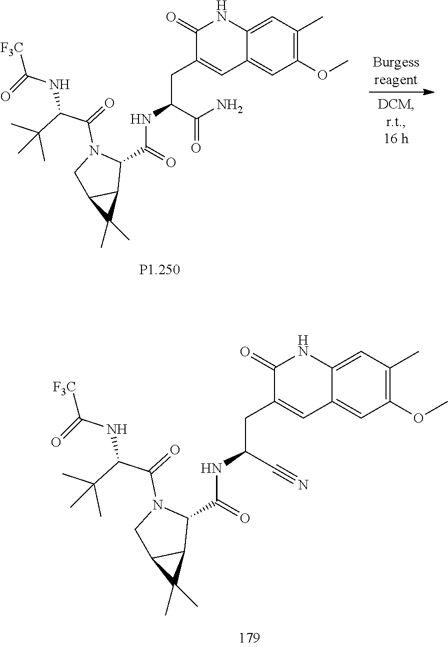 [SMILES: COc1cc2cc(C[C@H](NC(=O)[C@@H]3[C@@H]4[C@H](CN3C(=O)[C@@H](NC(=O)C(F)(F)F)C(C)(C)C)C4(C)C)C(N)=O)c(=O)[nH]c2cc1C ; COc1cc2cc(C[C@@H](C#N)NC(=O)[C@@H]3[C@@H]4[C@H](CN3C(=O)[C@@H](NC(=O)C(F)(F)F)C(C)(C)C)C4(C)C)c(=O)[nH]c2cc1C]

To a solution of P1.250 (107 mg, 0.172 mmol, 1.0 eq) in DCM (2 mL) was added Burgess reagent (102 mg, 0.430 mmol, 2.5 eq). The reaction solution was stirred at rt for 16 h. The reaction solution was concentrated under reduced pressure. The residue was purified by C18 flash column (5-100% methanol in H₂O) to afford compound 179 (50 mg, 48%) as a white solid. ¹H NMR (400 MHz, DMSO-d₆), δ: 11.84 (br. s, 1H), 9.40 (d, J=8.4 Hz, 1H), 8.90 (d, J=7.2 Hz, 1H), 7.82 (s, 1H), 7.14-7.10 (m, 2H), 5.09-5.06 (m, 1H), 4.39 (d, J=8.4 Hz, 1H), 4.14 (s, 1H), 4.09-3.93 (m, 1H), 3.80-3.62 (m, 4H), 3.16-1.15 (m, 1H), 3.04-3.00 (m, 2H), 2.21 (s, 3H), 1.54-1.51 (m, 1H), 1.10-0.79 (m, 16H); LCMS (ESI) m/z calc. for C₃₀H₃₆F₃N₅O₅ 603.27. found, 604.1 [M+H]⁺. HPLC purity: 98.88%, t_{R}=21.939 min.

### Example 53. (1R,2S,5S)—N—((S)-1-Cyano-2-(6-methyl-2-oxo-1,2-dihydroquinolin-3-yl)ethyl)-3-((S)-3,3-dimethyl-2-((2,2,2-trifluoroethyl)sulfonamido)butanoyl)-6,6-dimethyl-3-azabicyclo[3.1.0]hexane-2-carboxamide (Compound 195)

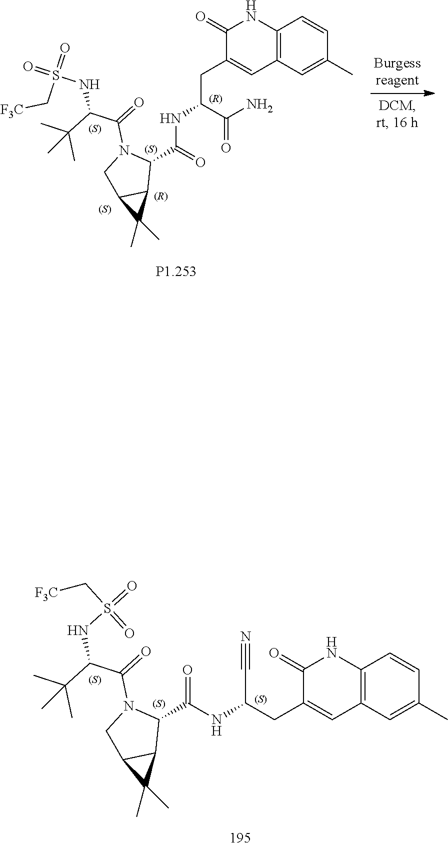

To a solution of P1.253 (1.00 g, 1.56 mmol) in DCM (16 mL) was added Burgess reagent (1.11 g, 4.68 mmol). After the mixture was stirred at rt for 16 h, the mixture was diluted with EtOAc. The solution was washed with NaHCO_{3(aq)} and brine. The organic layer was dried over MgSO₄, filtered, and concentrated under reduced pressure. The residue was purified by silica gel chromatography (0-5% MeOH in DCM with 1% NH₄OH_{(aq)}) to give compound 195 (885 mg, 91%) as white gum. ¹H NMR (400 MHz, DMSO-d₆), δ: 11.88 (s, 1H), 9.57-8.92 (m, 1H), 7.83 (s, 1H), 7.72 (d, J=8.4 Hz, 1H), 7.43 (s, 1H), 7.35-7.28 (m, 1H), 7.21 (d, J=8.0 Hz, 1H), 5.13-5.00 (m, 1H), 4.45-4.27 (m, 1H), 4.23-4.07 (m, 2H), 3.91-3.79 (m, 2H), 3.72-3.64 (m, 1H), 3.13-2.94 (m, 2H), 2.32 (s, 3H), 1.58-1.50 (m, 1H), 1.40-1.09 (m, 1H), 1.05-0.82 (m, 15H); LCMS (ESI) m/z calc. for C₂₉H₃₆F₃N₅O₅S 623.24. found, 624.1 [M+H]; HPLC purity: 98.8%, t_{R}=21.467 min.

### Example 54. (1R,2S,5S)—N—((S)-1-Cyano-2-(6-methyl-2-oxo-1,2-dihydroquinolin-3-yl)ethyl)-3-(4-methoxybenzofuran-2-carbonyl)-6,6-dimethyl-3-azabicyclo[3.1.0]hexane-2-carboxamide (Compound 196)

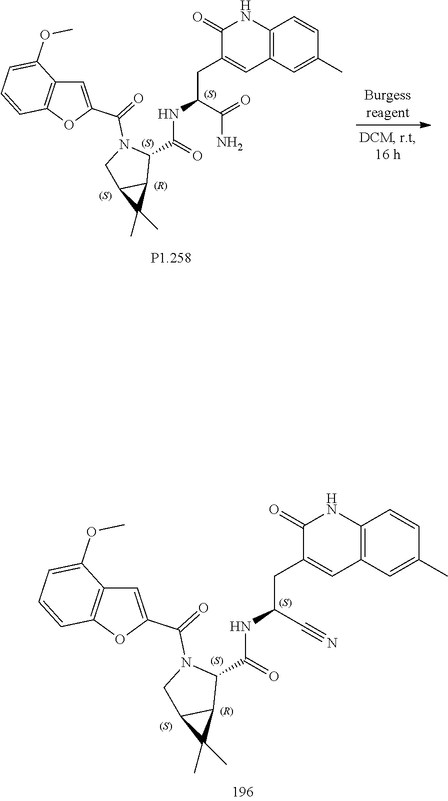

To a solution of P1.258 (200 mg, 0.359 mmol) in dichloromethane (3.6 mL) was added Burgess reagent (257 mg, 1.08 mmol). After the mixture was stirred at room temperature for 16 h, the mixture was concentrated under reduced pressure. The residue was purified by C18 reverse phase chromatography (0-100% MeOH in H₂O) to give compound 196 (112 mg, 58%) as a white solid. ¹H NMR (400 MHz, DMSO-d₆): δ 11.91-11.76 (m, 1H), 9.24-8.81 (m, 1H), 7.81-7.61 (m, 1H), 7.50-6.93 (m, 6H), 6.93-6.75 (m, 1H), 5.20-5.05 (m, 1H), 4.73-4.30 (m, 1H), 4.30-3.71 (m, 5H), 3.17-2.89 (m, 2H), 2.33-2.25 (m, 3H), 1.55-1.20 (m, 1H), 1.19-0.96 (m, 1H), 0.89-0.78 (m, 6H); LCMS (ESI) m/z calc. for C₃₁H₃₀N₄O₅ 538.22. found, 539.1 [M+H]⁺; HPLC purity: 100.0%, t_{R}=20.794 min.

### Example 55. Benzyl ((2S)-1-((3aR,6aR)-5-(((S)-1-cyano-2-(6-methyl-2-oxo-1,2-dihydroquinolin-3-yl)ethyl)carbamoyl)hexahydro-4H-furo[3,2-b]pyrrol-4-yl)-3,3-dimethyl-1-oxobutan-2-yl)carbamate (Compound 197)

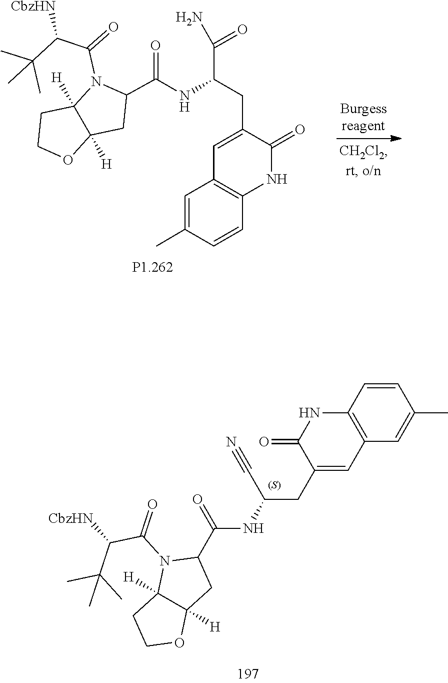

To a solution of P1.262 (92 mg, 0.146 mmol) in DCM (2.0 mL) was added methyl N-(triethylammoniumsulfonyl)carbamate (121.5 mg, 0.511 mmol). The resulting mixture was stirred at rt for overnight. The reaction mixture was diluted with water and extracted by DCM. The combined organic layers were dried over Na₂SO₄₍ₛ₎, filtered, and concentrated to obtain a residue. It was purified by silica gel column chromatography (0-20% DCM in methanol) to give target product 197 (68 mg, 76% yield). ¹H NMR (400 MHz, CDCl₃), δ: 11.17-8.45 (m, 2H), 7.77-7.60 (m, 1H), 7.31-6.77 (m, 9H), 6.14-5.40 (m, 1H), 5.30-4.25 (m, 7H), 4.11-2.80 (m, 6H), 2.38-2.34 (m, 4H), 2.23-1.94 (m, 2H), 1.04-0.93 (m, 9H). ¹H NMR (400 MHz, CD₃OD), δ: 7.89-7.77 (m, 1H), 7.46-7.17 (m, 8H), 5.17-5.03 (m, 3H), 4.83-4.27 (m, 4H), 4.03-3.90 (m, 1H), 3.58-3.41 (m, 1H), 3.19-3.12 (m, 2H), 2.89-2.65 (m, 1H), 2.42-2.37 (m, 4H), 2.16-2.11 (m, 2H), 1.08-0.96 (m, 9H). LRMS (ESI) m/z 614.1 [M+H]⁺.

### Example 56. (3aR,6aR)—N—((S)-1-Cyano-2-(6-methyl-2-oxo-1,2-dihydroquinolin-3-yl)ethyl)-4-((S)-3,3-dimethyl-2-(2,2,2-trifluoroacetamido)butanoyl)hexahydro-2H-furo[3,2-b]pyrrole-5-carboxamide (Compound 198)

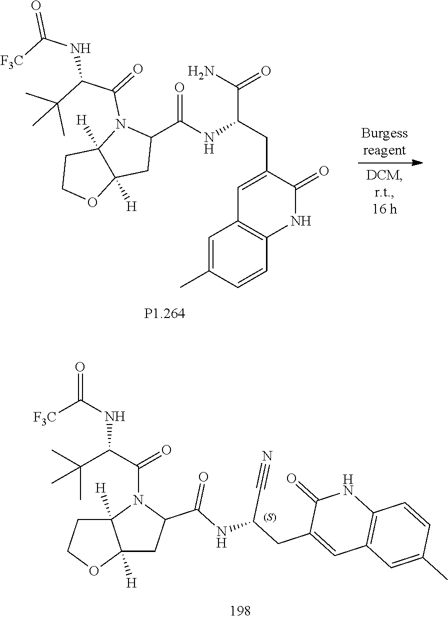

To a solution of P1.264 (63.0 mg, 0.106 mmol, 1.0 eq) in DCM (3 mL) was added Burgess reagent (63.3 mg, 0.265 mmol, 2.5 eq) and the reaction solution was stirred at rt for 16 h. The reaction solution was concentrated under reduced pressure and purified by C18 flash column chromatography (5-100% methanol in H₂O) to afford compound 198 (53.6 mg, 88%) as a white solid. ¹H NMR (400 MHz, CDCl₃), δ: 10.99-10.67 (m, 1H), 9.57 (s, 0.4H), 8.92-8.58 (m, 1H), 7.78-7.75 (m, 1H), 7.40-7.30 (m, 2H), 7.18-7.12 (m, 2H), 5.02-4.68 (m, 3H), 4.61-4.55 (m, 0.7H), 4.52-4.47 (m, 1H), 4.41-4.38 (m, 1H), 4.12-3.49 (m, 2H), 3.30-3.07 (m, 2H), 2.86-2.79 (m, 2H), 2.67-2.63 (m, 0.5H), 2.40-2.39 (m, 4H), 2.35-2.20 (m, 2H), 2.04-2.03 (m, 0.5H), 1.09-1.07 (m, 6H), 1.00-0.96 (m, 4H); LCMS (ESI) m/z calc. for C₂₈H₃₂F₃N₅O₅ 575.24. found, 576.1 [M+H]⁺; HPLC purity: 93.3%, t_{R}=19.043 and 19.972 min.

### Example 57. (1R,2S,5S)—N-(1-cyano-2-(6,7-dimethyl-2-oxo-1,2-dihydroquinolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 145)

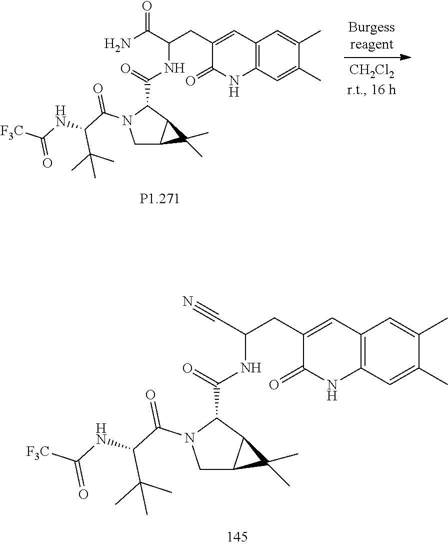 [SMILES: Cc1cc2cc(CC(NC(=O)[C@@H]3[C@@H]4[C@H](CN3C(=O)[C@@H](NC(=O)C(F)(F)F)C(C)(C)C)C4(C)C)C(N)=O)c(=O)[nH]c2cc1C ; "P1.271" ; Cc1cc2cc(CC(C#N)NC(=O)[C@@H]3[C@@H]4[C@H](CN3C(=O)[C@@H](NC(=O)C(F)(F)F)C(C)(C)C)C4(C)C)c(=O)[nH]c2cc1C]

To a solution of P1.271 (340 mg, 0.56 mmol, 1.0 eq) in DCM (3.7 mL) was added Burgess reagent (334 mg, 1.40 mmol, 2.5 eq). The resulting mixture was stirred at rt for 16 h. The mixture was concentrated under reduced pressure and purified by C-18 reversed-phase column chromatography (0-100% MeOH in water containing 1% NH₄HCO₃) to give compound 145 (272.2 mg, 82% yield) as a yellow solid. ¹H NMR (400 MHz, DMSO-d₆), δ: 11.81-11.80 (m, 2H), 9.42-9.37 (m, 2H), 8.99-8.85 (m, 2H), 7.77-7.76 (m, 2H), 7.38-7.36 (m, 2H), 7.07-7.06 (m, 2H), 5.13-5.03 (m, 2H), 4.40-4.35 (m, 2H), 4.14-4.13 (m, 2H), 3.97-3.79 (m, 2H), 3.63-3.59 (m, 2H), 3.06-2.89 (m, 4H), 2.26-2.22 (m, 12H), 1.53-1.15 (m, 3H), 0.98-0.84 (m, 25H), 0.78 (s, 6H); LCMS (ESI) m/z calc. for C₃₀H₃₆F₃N₅O₄ 587.27. found, 588.2 [M+H]⁺; HPLC purity: 99.5%, t_{R}=22.55 min.

### Example 58. (1R,2S,5S)—N-[(1S)-1-Cyano-2-(6-methyl-2-oxo-3,4-dihydro-1H-quinolin-3-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 120)

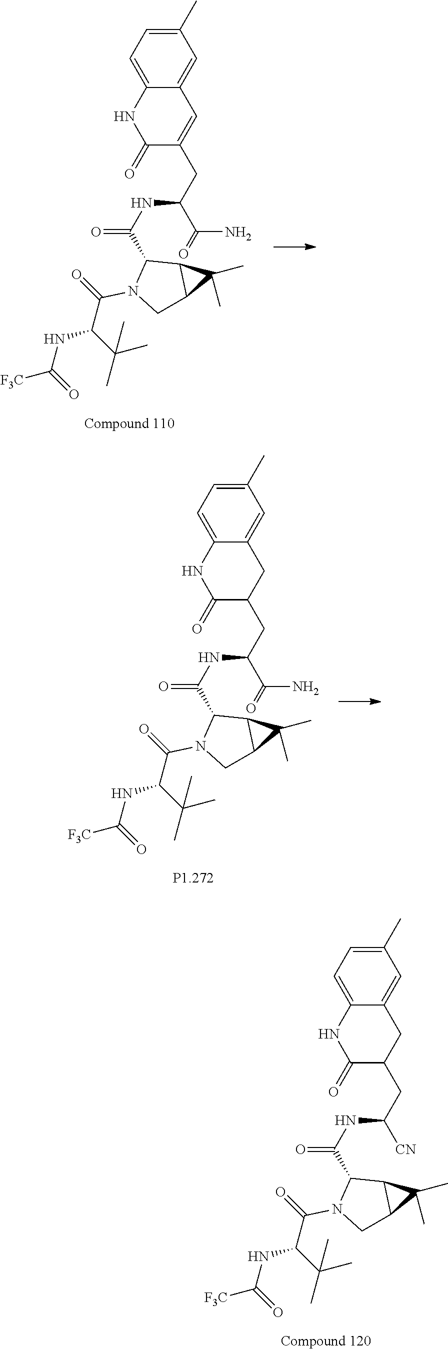 [SMILES: Cc1ccc2[nH]c(=O)c(C[C@H](NC(=O)[C@@H]3[C@@H]4[C@H](CN3C(=O)[C@@H](NC(=O)C(F)(F)F)C(C)(C)C)C4(C)C)C(N)=O)cc2c1 ; Cc1ccc2c(c1)CC(C[C@H](NC(=O)[C@@H]1[C@@H]3[C@H](CN1C(=O)[C@@H](NC(=O)C(F)(F)F)C(C)(C)C)C3(C)C)C(N)=O)C(=O)N2 ; Cc1ccc2c(c1)CC(C[C@@H](C#N)NC(=O)[C@@H]1[C@@H]3[C@H](CN1C(=O)[C@@H](NC(=O)C(F)(F)F)C(C)(C)C)C3(C)C)C(=O)N2]

A mixture of compound 110 (300 mg), 10% Pd—C (30 mg) and ethanol (10 ml) was heated in an autoclave at 90° C. under 50 atmospheres of H₂ for 15 h. The catalyst was removed by filtration and the filtrate was concentrated in vacuo to afford P1.272 (300 mg) as an off-white solid, which was used in the next stage without purification. (Another option for preparation of the compound P1.272 and analytical data presented in Preparation 271). To a solution of P1.272 (300 mg) in 5 ml DCM Burgess reagent (300 mg, 2.5 eq) was added. Reaction mixture was stirred at rt for 15 h, washed with water, saturated NaHCO₃ and 5% HCl, dried over Na₂SO₄, filtered, and concentrated under reduced pressure. The residue was purified by prep RP-HPLC on a C18 column eluting with a gradient MeCN-H2O+0.1% TFA to yield the compound 120 (39 mg, 13%). ¹H NMR (400 MHz, DMSO-d₆), δ: 10.11 (s, 1H), 9.33 (d, J=8.4 Hz, 1H), 8.91 (d, J=7.6 Hz, 1H), 7.01-6.90 (m, 2H), 6.78-6.67 (m, 1H), 5.12 (q, J=7.7 Hz, 1H), 4.45-4.33 (m, 1H), 4.16 (s, 1H), 3.89 (dd, J=10.5, 5.4 Hz, 1H), 3.65 (d, J=10.3 Hz, 1H), 2.87 (dd, J=15.9, 6.3 Hz, 1H), 2.76-2.65 (m, 1H), 2.37 (dd, J=13.8, 6.4 Hz, 1H), 2.20 (s, 3H), 1.82 (ddd, J=14.2, 9.1, 5.9 Hz, 1H), 1.56 (dd, J=7.7, 5.3 Hz, 1H), 1.29 (d, J=7.6 Hz, 1H), 1.02 (s, 3H), 0.96 (d, J=8.1 Hz, 1H), 0.90-0.80 (m, 11H), 0.80 (d, J=9.6 Hz, 1H).

### Example 59. (1R,2S,5S)—N-[1-Cyano-2-(6-methyl-2-oxo-3,4-dihydro-1H-quinolin-3-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 199)

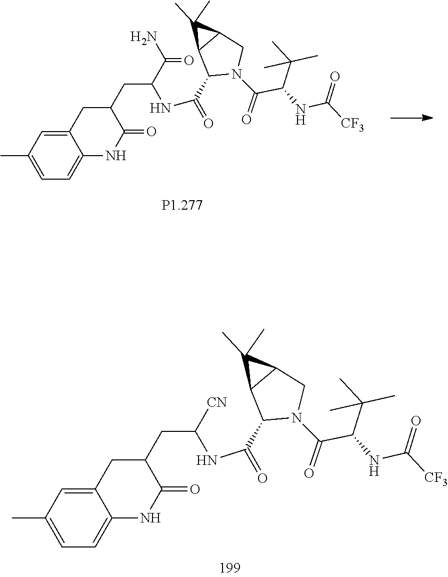 [SMILES: Cc1ccc2c(c1)CC(CC(NC(=O)[C@@H]1[C@@H]3[C@H](CN1C(=O)[C@@H](NC(=O)C(F)(F)F)C(C)(C)C)C3(C)C)C(N)=O)C(=O)N2 ; Cc1ccc2c(c1)CC(CC(C#N)NC(=O)[C@@H]1[C@@H]3[C@H](CN1C(=O)[C@@H](NC(=O)C(F)(F)F)C(C)(C)C)C3(C)C)C(=O)N2]

To a solution of Compound P1.277 (240 mg, 0.4 mmol) in 10 ml of DCM Burgess reagent (240 mg, 1 mmol) was added. Reaction mixture was stirred at rt for 15 h, washed with water, saturated NaHCO₃ and 5% HCl, dried over Na₂SO₄, filtered, and concentrated under reduced pressure. The residue was purified by prep RP-HPLC on a C18 column eluting with a gradient MeCN-H₂O+0.1% TFA. Yield of compound 199: 38 mg, 16%.

### Analytical Data of Certain Final Compounds

Compound 200. Methyl N-[(1R)-1-[(1S)-2-[[(1R)-1-cyano-2-(6-methyl-2-oxo-1H-quinolin-3-yl)ethyl]carbamoyl]-6,6-dimethyl-3-azabicyclo[3.1.0]hexane-3-carbonyl]-2,2-dimethyl-propyl]carbamate. ¹H NMR (400 MHz, DMSO-d₆), δ: 11.88 (d, J=6.8 Hz, 1H), 7.81 (s, 1H), 7.42 (d, J=9.6 Hz, 1H), 7.32 (d, J=8.8 Hz, 1H), 7.23-7.19 (m, 1H), 5.16-5.05 (m, 1H), 4.13 (s, 1H), 4.05-4.00 (m, 1H), 3.92-3.84 (m, 1H), 3.79-3.71 (m, 1H), 3.50 (s, 3H), 3.03 (d, J=6.8 Hz, 6H), 2.32 (s, 3H), 1.54-1.49 (m, 1H), 1.17-1.14 (m, 1H), 0.93 (s, 9H), 0.82 (s, 3H).

Compound 201. Ethyl N-[(1R)-1-[(1S)-2-[[(1R)-1-cyano-2-(6-methyl-2-oxo-1H-quinolin-3-yl)ethyl]carbamoyl]-6,6-dimethyl-3-azabicyclo[3.1.0]hexane-3-carbonyl]-2,2-dimethyl-propyl]carbamate. ¹H NMR (400 MHz, DMSO-d₆), δ: 11.88 (s, 1H), 8.83 (d, J=7.3 Hz, 1H), 7.81 (s, 1H), 7.43 (d, J=8.7 Hz, 1H), 7.32 (dd, J=8.3, 1.8 Hz, 1H), 7.21 (dd, J=8.4, 4.0 Hz, 1H), 7.11-7.00 (m, 1H), 5.19-5.02 (m, 1H), 4.14 (s, 1H), 4.11-3.67 (m, 5H), 3.14-2.88 (m, 2H), 2.32 (s, 3H), 1.56-1.45 (m, 1H), 1.13 (t, J=7.1 Hz, 3H), 1.09-1.02 (m, 1H), 0.92 (s, 12H), 0.83 (s, 3H).

Compound 202. Isopropyl N-[(1R)-1-[(1S)-2-[[(1R)-1-cyano-2-(6-methyl-2-oxo-1H-quinolin-3-yl)ethyl]carbamoyl]-6,6-dimethyl-3-azabicyclo[3.1.0]hexane-3-carbonyl]-2,2-dimethyl-propyl]carbamate. ¹H NMR (400 MHz, DMSO-d₆), δ: 11.93-11.82 (m, 1H), 9.06-8.74 (m, 1H), 7.81 (s, 1H), 7.42 (d, J=8.5 Hz, 1H), 7.32 (d, J=8.4 Hz, 1H), 7.21 (dd, J=8.4, 3.8 Hz, 1H), 7.01-6.89 (m, 1H), 5.19-5.02 (m, 1H), 4.79-4.61 (m, 1H), 4.14 (s, 1H), 4.11-3.96 (m, 1H), 3.96-3.69 (m, 2H), 3.14-2.87 (m, 2H), 2.32 (s, 3H), 1.57-1.45 (m, 1H), 1.20-1.02 (m, 7H), 0.92 (s, 12H), 0.82 (s, 3H).

Compound 203. (1S)—N-[(1R)-1-Cyano-2-(6-methyl-2-oxo-1H-quinolin-3-yl)ethyl]-3-[(2R)-2-(cyclopropylsulfonylamino)-3,3-dimethyl-butanoyl]-6,6-dimethyl-3-azabicyclo[3.1.0]hexane-2-carboxamide. ¹H NMR (400 MHz, DMSO-d₆), δ: 11.92-11.80 (m, 1H), 9.06-8.89 (m, 1H), 7.88-7.78 (m, 1H), 7.43 (d, J=9.0 Hz, 1H), 7.32 (dt, J=8.3, 2.5 Hz, 1H), 7.21 (dd, J=8.4, 6.0 Hz, 1H), 7.02-6.87 (m, 1H), 5.20-5.01 (m, 1H), 4.23-4.12 (m, 1H), 3.93-3.58 (m, 3H), 3.14-2.87 (m, 2H), 2.33 (s, 3H), 1.58-1.48 (m, 1H), 1.34-1.20 (m, 1H), 1.13 (d, J=7.7 Hz, 1H), 1.08-0.73 (m, 19H).

Compound 204. (1S)—N-[(1R)-1-cyano-2-(6-methyl-2-oxo-1H-quinolin-3-yl)ethyl]-3-[(2R)-3,3-dimethyl-2-(2-methylpropanoylamino)butanoyl]-6,6-dimethyl-3-azabicyclo[3.1.0]hexane-2-carboxamide. ¹H NMR (400 MHz, DMSO-d₆), δ: 11.89 (s, 1H), 8.74 (d, J=7.4 Hz, 1H), 7.86-7.72 (m, 2H), 7.43 (d, J=7.5 Hz, 1H), 7.32 (dd, J=8.4, 1.9 Hz, 1H), 7.21 (dd, J=8.4, 2.1 Hz, 1H), 5.20-5.03 (m, 1H), 4.45-4.31 (m, 1H), 4.16-4.06 (m, 1H), 3.83-3.59 (m, 2H), 3.13-2.88 (m, 2H), 2.72-2.43 (m, 1H), 2.32 (s, 3H), 1.53-1.42 (m, 1H), 1.31-1.11 (m, 1H), 1.10-0.80 (m, 18H), 0.75 (s, 3H).

Compound 206. (2S)—N-[(1S)-1-benzyl-2-[[(1S)-1-cyano-2-(6-methyl-2-oxo-1H-quinolin-3-yl)ethyl]amino]-2-oxo-ethyl]-N,3,3-trimethyl-2-[(2,2,2-trifluoroacetyl)amino]butanamide. ¹H NMR (400 MHz, CD₃OD), δ: 7.90-7.02 (m, 9H), 5.36-5.02 (m, 2H), 4.80-4.71 (m, 1H), 3.29-3.12 (m, 2H), 3.10-2.83 (m, 5H), 2.46-2.36 (m, 3H), 0.99-0.75 (m, 9H).

Compound 207. (2S)—N-[(1S)-1-benzyl-2-[[(1R)-1-cyano-2-(6-methyl-2-oxo-1H-quinolin-3-yl)ethyl]amino]-2-oxo-ethyl]-N,3,3-trimethyl-2-[(2,2,2-trifluoroacetyl)amino]butanamide. ¹H NMR (400 MHz, CD₃OD), δ: 7.94-7.78 (m, 1H), 7.54-7.32 (m, 2H), 7.32-7.09 (m, 6H), 5.58-5.39 (m, 2H), 5.22-5.12 (m, 1H), 4.83-4.56 (m, 1H), 3.74-3.32 (m, 1H), 3.27-3.00 (m, 2H), 2.99-2.86 (m, 4H), 2.47-2.34 (m, 3H), 0.93-0.61 (m, 9H).

Compound 208. (1R,2S,5S)—N-[(1R)-1-cyano-2-(6-methyl-2-oxo-1H-quinolin-3-yl)ethyl]-3-[(2S)-3,3-dimethyl-2-[(2,2,2-trifluoroacetyl)amino]butanoyl]-N,6,6-trimethyl-3-azabicyclo[3.1.0]hexane-2-carboxamide. ¹H NMR (400 MHz, DMSO-d₆), δ: 12.01-11.79 (m, 1H), 9.60-9.11 (m, 1H), 7.96-7.70 (m, 1H), 7.49-7.12 (m, 3H), 5.63 (t, J=8.0 Hz, 1H), 4.58-4.28 (m, 2H), 3.88-3.59 (m, 2H), 3.22-2.88 (m, 5H), 2.36-2.27 (m, 3H), 1.52-1.26 (m, 1H), 1.25-1.04 (m, 2H), 1.01-0.50 (m, 14H).

Compound 209. (1R,2S,5S)—N-[(1S)-1-cyano-2-(6-methyl-2-oxo-1H-quinolin-3-yl)ethyl]-3-[(2S)-3,3-dimethyl-2-[(2,2,2-trifluoroacetyl)amino]butanoyl]-N,6,6-trimethyl-3-azabicyclo[3.1.0]hexane-2-carboxamide. ¹H NMR (400 MHz, DMSO-d₆), δ: 11.82 (s, 1H), 9.30 (d, J=7.8 Hz, 1H), 7.86 (s, 1H), 7.38 (s, 1H), 7.29 (dd, J=8.4, 2.0 Hz, 1H), 7.18 (d, J=8.4 Hz, 1H), 5.84 (t, J=7.8 Hz, 1H), 4.52 (s, 1H), 4.33 (d, J=7.6 Hz, 1H), 3.68-3.57 (m, 2H), 3.15-3.06 (m, 5H), 2.32-2.28 (m, 3H), 1.24-1.20 (m, 1H), 0.98 (s, 10H), 0.79 (s, 3H), 0.73 (s, 3H).

### Biological Assays

### Example A. Primary Assay Used to Determine Potency of 3CL Protease Enzymatic Activity

For determination of 3CL Protease enzymatic activity was used Assay Kit of BPS Bioscience, San Diego, CA (Catalog #78043-1).

All samples and controls should be tested in duplicate.
1) Just before use, dilute 0.5 M DTT 500 times into the 3CL Protease Assay Buffer to obtain a DTT concentration of 1 mM. For example, add 10 μl of 0.5 M DTT to 5 ml of assay buffer. Prepare enough DTT-containing buffer as required for the assay. Store the remaining assay buffer at −20° C.
2) Thaw the 3CL Protease on ice. Briefly spin the tube containing the enzyme to recover the full content of the tube. Note: 3CL Protease enzyme is sensitive to freeze/thaw cycles. Do not re-use the diluted enzyme.
3) Dilute 3CL Protease in Assay buffer (containing 1 mM DTT) at 0.5 ng/μl (15 ng per reaction). Note: The exact concentration and volume of enzyme is lot-specific and will be indicated on the tube. Calculate required dilution from the information in the tube. It may be desirable to dilute the enzyme serially to avoid using large amounts of protease assay buffer for the dilution.
4) Add 30 μl of diluted 3CL Protease to the wells designated as “Positive Control,” “Inhibitor Control,” and “Test Inhibitor.” Add 30 μl of Assay Buffer (containing 1 mM DTT) to the “Blank” wells.
5) Dilute the 50 μg GC376 in 200 μl of Assay Buffer to obtain a 500 μM solution. Add 10 μl of GC376 (500 μM) to the wells labeled “Inhibitor Control”. Aliquot and store the remaining solution at −80° C.
6) Prepare the Test Inhibitor. The final concentration of DMSO in the assay should not exceed 1%. If the Test Inhibitor is dissolved in DMSO, make a 100-fold higher concentration of the compound than the highest desired concentration. Then make a 20-fold dilution in 1× Assay Buffer (containing DTT). At this step the compound concentration is 5-fold higher than the final concentration. If the test inhibitor is dissolved in water, make a 5-fold higher concentration of the test compound than the final desired concentration in the Assay buffer (containing DTT). Add 10 μl of Test Inhibitor to each well designated “Test Inhibitor”.
7) Diluent Solution (no inhibitor): add 10 μl of assay buffer (if the test compound is water soluble and was diluted in assay buffer) or 10 μl of 5% DMSO diluted in assay buffer (if DMSO was used to dissolve the test inhibitor) to “Blank” and “Positive Control” wells.

| | Positive | Test | Inhibitor | |
|---|---|---|---|---|
| Component | control | inhibitor | control | Blank |
| 3CL Protease (0.5 ng/μl) | 30 μl | 30 μl | 30 μl | — |
| Assay Buffer (with DTT) | — | — | — | 30 μl |
| GC376 (500 μM) | — | — | 10 μl | — |
| Test Inhibitor | — | 10 μl | — | — |
| Diluent solution | 10 μl | — | — | 10 μl |
| Total | 40 μl | 40 μl | 40 μl | 40 μl |

8) Preincubate for 30 min at room temperature with slow shaking.
9) Dilute the 25 μl of 3CL Protease substrate (10 mM) in 1.25 ml of the Assay Buffer containing DTT, to make a 200 M solution. The final concentration of the 3CL Protease substrate in a 50 μl reaction is 40 μM.
10) Start the reaction by adding 10 μl of the substrate solution to all the wells. Seal the plate with the plate sealer and incubate for 4 hours at room temperature with slow shaking.
11) Measure the fluorescence intensity in a microtiter plate-reader fluorometer (Tecan Spark Cyto) capable of excitation at a wavelength 360 nm and detection of emission at a wavelength 460 nm. The fluorescence intensity can also be measured kinetically. The “Blank” value is subtracted from all other values.
The % inhibition was then used to calculate the IC₅₀ values. The IC₅₀ values are shown in Table A, wherein “A” corresponds to IC₅₀<50.0 nm, “B” corresponds to 50.0 nm≤IC₅₀<100.0 nm, “C” corresponds to 100.0 nm≤IC₅₀<250.0 nm, “D” corresponds to 250.0 nm≤IC₅₀<500.0 nm, and “E” corresponds to 500.0 nm≤IC₅₀<1000.0 nm, and “F” corresponds to 1000.0 nm≤IC₅₀.

TABLE A
3CL Protease enzymatic activity

| Compound | |
|---|---|
| Number | IC₅₀, nM* |
| 101 | C |
| 102 | A |
| 104 | B |
| 105 | B |
| 106 | C |
| 107 | B |
| 109 | D |
| 112 | F |
| 113 | A |
| 114 | F |
| 120 | A |
| 121 | D |
| 122 | C |
| 123 | C |
| 124 | C |
| 125 | D |
| 126 | C |
| 127 | E |
| 128 | E |
| 130 | A |
| 145 | B |
| 146 | B |
| 147 | A |
| 148 | F |
| 151 | E |
| 152 | C |
| 153 | F |
| 156 | F |
| 157 | D |
| 158 | B |
| 159 | F |
| 160 | F |
| 161 | F |
| 164 | C |
| 166 | E |
| 171 | D |
| 173 | F |
| 174 | A |
| 175 | B |
| 176 | B |
| 177 | B |
| 178 | C |
| 179 | B |
| 182 | A |
| 183 | F |
| 185 | A |
| 186 | A |
| 187 | F |
| 188 | A |
| 189 | F |
| 191 | F |
| 192 | F |
| 193 | F |
| 194 | F |
| 195 | A |
| 196 | F |
| 197 | F |
| 198 | F |
| 199 | A |
| 200 | A |
| 201 | B |
| 202 | C |
| 203 | B |
| 204 | B |
| 205 | F |
| 206 | F |

*IC50 is a quantitative measure that indicates how much of a particular inhibitory substance (e.g. drug) is needed to inhibit, in vitro, a given biological process or biological component by 50%; wherein “A” corresponds to IC50 < 50.0 nm, “B” corresponds to 50.0 nm ≤ IC50 < 100.0 nm, “C” corresponds to 100.0 nm ≤ IC50 < 250.0 nm, “D” corresponds to 250.0 nm ≤ IC50 < 500.0 nm, and “E” corresponds to 500.0 nm ≤ IC50 < 1000.0 nm, and “F” corresponds to 1000.0 nm ≤ IC50.

### Example B. Comparative Example

In the Table B presented comparative data on 3CL Protease enzymatic activity of the compounds of present disclosure and known compound K:

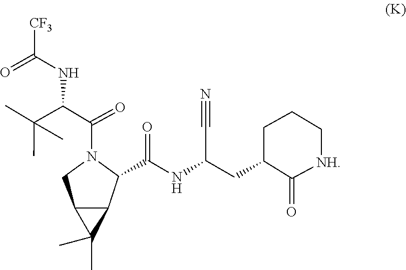 [SMILES: CC(C)(C)[C@H](NC(=O)C(F)(F)F)C(=O)N1C[C@H]2[C@@H]([C@H]1C(=O)N[C@H](C#N)C[C@@H]1CCCNC1=O)C2(C)C]

TABLE B

| Compound | |
|---|---|
| Number | IC₅₀, nM* |
| K | 1294 |
| 101 | 147 |
| 104 | 89 |
| 123 | 180 |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (A): 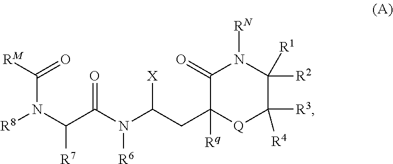
or a pharmaceutically acceptable salt, stereoisomer, solvate, prodrug, or tautomer thereof, wherein:
each R^{N} is independently selected from H, C₁-C₆ alkyl, cycloalkyl;
Q is CR⁵R^{Q} or NR^{Q};
R^{q} is H;
R^{Q} is R^{N}; or
R^{q} and R^{Q} together form a bond;
R^{M} is selected from 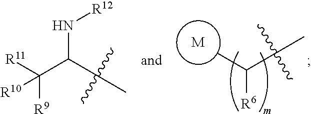
M is selected from a 5-10 membered heterocyclyl or a 5-10 membered heteroaryl, wherein the heterocyclyl or heteroaryl is optionally substituted with one or more R¹⁶;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R² is selected from hydrogen, halogen, —OH, —CN, —NO₂, —NR¹⁴R¹⁵, C₁-C₆ alkyl, C₂-C₆alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R² together are oxo;
or R² and R³ together form a bond;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁵ is selected from hydrogen, halogen, —OH, —CN, —NO₂, —NR¹⁴R¹⁵, C₁-C₆ alkyl, C₂-C₆alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
each R⁶ is independently selected from H, C₁-C₆ alkyl, cycloalkyl, —CH₂-aryl, wherein the alkyl, cycloalkyl, or aryl is optionally substituted with one or more R¹⁶;
R⁷ is —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
or R⁷ and R⁸ together with the atoms to which they are attached and any intervening atoms, form 5-7 membered monocyclic or bicyclic heterocyclyl optionally substituted with one or more R¹³;
R⁹, R¹⁰, R¹¹ are each independently selected from C₁-C₆ alkyl, cycloalkyl, halogen, —CN;
R¹² is selected from R⁶—S(O)₂—, R⁶—C(O)—, R⁶—O—C(O)—, heteroaryl, wherein the heteroaryl is optionally substituted with one or more R¹⁶;
each R¹³ is independently selected from halogen, —OH, —CN, —C₁-C₆ alkyl, —C₁-C₆ halogenalkyl;
two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
R¹⁴ and R¹⁵ are each independently selected from H, C₁-C₆ alkyl, cycloalkyl;
or R¹⁴ and R¹⁵ together with the atoms to which they are attached and any intervening atoms, form heterocyclyl optionally substituted with one or more halogen, —OH, —CN, C₁-C₆ alkyl;
each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
n is an integer selected from 0, 1, and 2;
m is an integer selected from 0 and 1;
provided that when R^{q} is H then R¹ and R⁴ together with the atoms to which they are attached and any intervening atoms, form a 3-10 membered cycloalkyl, a 6-10 membered aryl, a 3-14 membered heterocycle, or a 5-6 membered heteroaryl;
wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

2. The compound of claim 1, wherein the compound is of Formula (I): 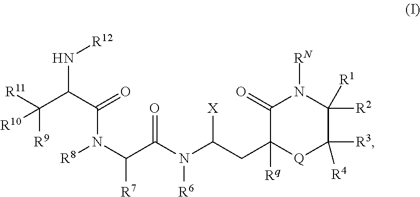
or a pharmaceutically acceptable salt, stereoisomer, solvate, prodrug, or tautomer thereof, wherein:
each R^{N} is independently selected from H, C₁-C₆ alkyl, cycloalkyl;
Q is CR⁵R^{Q} or NR^{Q};
R^{q} is H;
R^{Q} is R^{N}; or
R^{q} and R^{Q} together form a bond;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R² is selected from hydrogen, halogen, —OH, —CN, —NO₂, —NR¹⁴R¹⁵, C₁-C₆ alkyl, C₂-C₆alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R² together are oxo;
or R² and R³ together form a double bond;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁵ is selected from hydrogen, halogen, —OH, —CN, —NO₂, —NR¹⁴R¹⁵, C₁-C₆ alkyl, C₂-C₆alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
each R⁶ is independently selected from H, C₁-C₆ alkyl, cycloalkyl, —CH₂-aryl, wherein the alkyl, cycloalkyl, or aryl is optionally substituted with one or more R¹⁶;
R⁷ is —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
or R⁷ and R⁸ together with the atoms to which they are attached and any intervening atoms, form 5-7 membered monocyclic or bicyclic heterocyclyl optionally substituted with one or more R¹³;
R⁹, R¹⁰, R¹¹ are each independently selected from C₁-C₆ alkyl, cycloalkyl, halogen, —CN;
R¹² is selected from R⁶—S(O)₂—, R⁶—C(O)—, R⁶—O—C(O)—, heteroaryl, wherein the heteroaryl is optionally substituted with one or more R¹⁶;
each R¹³ is independently selected from halogen, —OH, —CN, —C₁-C₆ alkyl, —C₁-C₆ halogenalkyl;
two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
R¹⁴ and R¹⁵ are each independently selected from H, C₁-C₆ alkyl, cycloalkyl;
or R¹⁴ and R¹⁵ together with the atoms to which they are attached and any intervening atoms, form heterocyclyl optionally substituted with one or more halogen, —OH, —CN, C₁-C₆ alkyl;
each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
n is an integer selected from 0, 1, and 2.

3. The compound of claim 1, wherein the compound is of Formula (II): 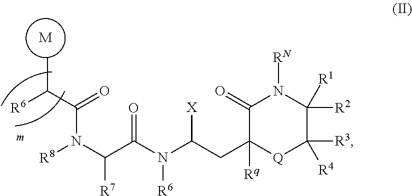
or a pharmaceutically acceptable salt, stereoisomer, solvate, prodrug, or tautomer thereof, wherein:
each R^{N} is independently selected from H, C₁-C₆ alkyl, cycloalkyl;
Q is CR⁵R^{Q} or NR^{Q};
R^{q} is H;
R^{Q} is R^{N}; or
R^{q} and R^{Q} together form a bond;
M is selected from 5-10 membered heterocyclyl or 5-10 membered heteroaryl, wherein the heterocyclyl or heteroaryl is optionally substituted with one or more R¹⁶;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R² together are oxo;
or R² and R³ together form a double bond;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁵ is selected from hydrogen, halogen, —OH, —CN, —NO₂, —NR¹⁴R¹⁵, C₁-C₆ alkyl, C₂-C₆alkenyl, C₂-C₆ alkynyl, C₁-C₆ alkoxy, —C(O)OR¹⁴, —C(O)N—NR¹⁴R¹⁵, cycloalkyl, —O— cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
each R⁶ is independently selected from H, C₁-C₆ alkyl, cycloalkyl, —CH₂-aryl, wherein the alkyl, cycloalkyl, or aryl is optionally substituted with one or more R¹⁶;
R⁷ is —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
or R⁷ and R⁸ together with the atoms to which they are attached and any intervening atoms, form 5-7 membered monocyclic or bicyclic heterocyclyl optionally substituted with one or more R¹³;
each R¹³ is independently selected from halogen, —OH, —CN, —C₁-C₆ alkyl, —C₁-C₆ halogenalkyl;
two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
R¹⁴ and R¹⁵ are each independently selected from H, C₁-C₆ alkyl, cycloalkyl;
or R¹⁴ and R¹⁵ together with the atoms to which they are attached and any intervening atoms, form heterocyclyl optionally substituted with one or more halogen, —OH, —CN, C₁-C₆ alkyl;
each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
m is an integer selected from 0 and 1;
n is an integer selected from 0, 1, 2.

4. The compound of claim 1, wherein the compound is of Formula (A′): 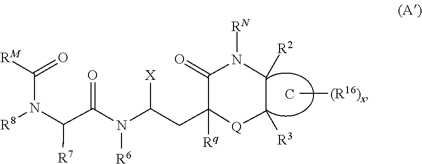
or a pharmaceutically acceptable salt, stereoisomer, solvate, prodrug, or tautomer thereof, wherein:
each R^{N} is independently selected from H, C₁-C₆ alkyl, cycloalkyl;
Q is CR⁵R^{Q} or NR^{Q};
R^{q} is H;
R^{Q} is R^{N}; or
R^{q} and R^{Q} together form a bond;
R^{M} is selected from 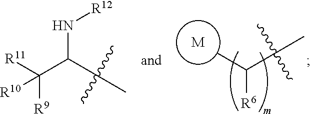
M is selected from 5-10 membered heterocyclyl or 5-10 membered heteroaryl, wherein the heterocyclyl or heteroaryl is optionally substituted with one or more R¹⁶;
C is a ring selected from a 3-10 membered cycloalkyl, a 6-10 membered aryl, a 3-14 membered heterocycle, or a 5-6 membered heteroaryl;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R² and R³ together form a bond;
R⁵ is selected from hydrogen, halogen, —OH, —CN, —NO₂, —NR¹⁴R¹⁵, C₁-C₆ alkyl, C₂-C₆alkenyl, C₂-C₆ alkynyl, C₁-C₆ alkoxy, —C(O)OR¹⁴, —C(O)N—NR¹⁴R¹⁵, cycloalkyl, —O— cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
each R⁶ is independently selected from H, C₁-C₆ alkyl, cycloalkyl, —CH₂-aryl, wherein the alkyl, cycloalkyl, or aryl is optionally substituted with one or more R¹⁶;
R⁷ is —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
or R⁷ and R⁸ together with the atoms to which they are attached and any intervening atoms, form 5-7 membered monocyclic or bicyclic heterocyclyl optionally substituted with one or more R¹³;
R⁹, R¹⁰, R¹¹ are each independently selected from C₁-C₆ alkyl, cycloalkyl, halogen, —CN;
R¹² is selected from R⁶—S(O)₂—, R⁶—C(O)—, R⁶—O—C(O)—, heteroaryl, wherein the heteroaryl is optionally substituted with one or more R¹⁶;
each R¹³ is independently selected from halogen, —OH, —CN, —C₁-C₆ alkyl, —C₁-C₆ halogenalkyl;
two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
R¹⁴ and R¹⁵ are each independently selected from H, C₁-C₆ alkyl, cycloalkyl;
or R¹⁴ and R¹⁵ together with the atoms to which they are attached and any intervening atoms, form heterocyclyl optionally substituted with one or more halogen, —OH, —CN, C₁-C₆ alkyl;
each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
n is an integer selected from 0, 1, 2;
m is an integer selected from 0 and 1;
x is an integer selected from 0, 1, 2, 3, and 4.

5. The compound of claim 2, wherein the compound is of Formula (I′): 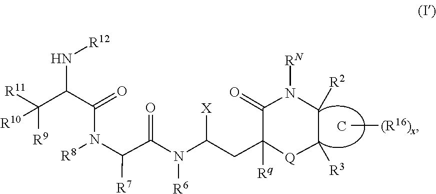
or a pharmaceutically acceptable salt, stereoisomer, solvate, prodrug, or tautomer thereof, wherein:
each R^{N} is independently selected from H, C₁-C₆ alkyl, cycloalkyl;
Q is CR⁵R^{Q} or NR^{Q};
R^{q} is H;
R^{Q} is R^{N}; or
R^{q} and R^{Q} together form a bond;
C is a ring selected from a 3-10 membered cycloalkyl, a 6-10 membered aryl, a 3-14 membered heterocycle, or a 5-6 membered heteroaryl;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R² and R³ together form a double bond;
R⁵ is selected from hydrogen, halogen, —OH, —CN, —NO₂, —NR¹⁴R¹⁵, C₁-C₆ alkyl, C₂-C₆alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
each R⁶ is independently selected from H, C₁-C₆ alkyl, cycloalkyl, —CH₂-aryl, wherein the alkyl, cycloalkyl, or aryl is optionally substituted with one or more R¹⁶;
R⁷ is selected from —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
or R⁷ and R⁸ together with the atoms to which they are attached and any intervening atoms, form 5-7 membered monocyclic or bicyclic heterocyclyl optionally substituted with one or more R¹³;
R⁹, R¹⁰, R¹¹ are each independently selected from C₁-C₆ alkyl, cycloalkyl, halogen, —CN;
R¹² is selected from R⁶—S(O)₂—, R⁶—C(O)—, R⁶—O—C(O)—, heteroaryl, wherein the heteroaryl is optionally substituted with one or more R¹⁶;
each R¹³ is independently selected from halogen, —OH, —CN, —C₁-C₆ alkyl, —C₁-C₆ halogenalkyl;
two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
R¹⁴ and R¹⁵ are each independently selected from H, C₁-C₆ alkyl, cycloalkyl;
or R¹⁴ and R¹⁵ together with the atoms to which they are attached and any intervening atoms, form heterocyclyl optionally substituted with one or more halogen, —OH, —CN, C₁-C₆ alkyl;
each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
n is an integer selected from 0, 1, 2;
x is an integer selected from 0, 1, 2, 3 and 4.

6. The compound of claim 3, wherein the compound is of Formula (II′): 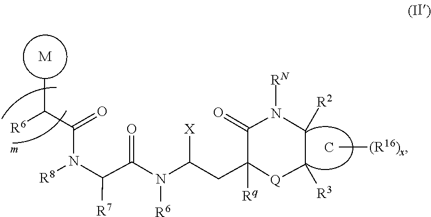
or a pharmaceutically acceptable salt, stereoisomer, solvate, prodrug, or tautomer thereof, wherein:
each R^{N} is independently selected from H, C₁-C₆ alkyl, cycloalkyl;
Q is CR⁵R^{Q} or NR^{Q};
R^{q} is H;
R^{Q} is R^{N}; or
R^{q} and R^{Q} together form a bond;
M is selected from a 5-10 membered heterocyclyl or a 5-10 membered heteroaryl, wherein the heterocyclyl or heteroaryl is optionally substituted with one or more R¹⁶;
C is a ring selected from cycloalkyl, aryl, heterocyclyl or heteroaryl;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R² and R³ together form a bond;
R⁵ is selected from hydrogen, halogen, —OH, —CN, —NO₂, —NR¹⁴R¹⁵, C₁-C₆ alkyl, C₂-C₆alkenyl, C₂-C₆ alkynyl, C₁-C₆ alkoxy, —C(O)OR¹⁴, —C(O)N—NR¹⁴R¹⁵, cycloalkyl, —O— cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
each R⁶ is independently selected from H, C₁-C₆ alkyl, cycloalkyl, —CH₂-aryl, wherein the alkyl, cycloalkyl, or aryl is optionally substituted with one or more R¹⁶;
R⁷ is selected from —(CH₂)ₙaryl, wherein aryl is optionally substituted with one or more substitutents independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl;
R⁸ is selected from hydrogen, C₁-C₆ alkyl, C₂-C₆ alkenyl, C₂-C₆ alkynyl, C₁-C₆ alkoxy, cycloalkyl, aryl, heterocyclyl, and heteroaryl;
or R⁷ and R⁸ together with the atoms to which they are attached and any intervening atoms, form 5-7 membered monocyclic or bicyclic heterocyclyl optionally substituted with one or more R¹³;
each R¹³ is independently selected from halogen, —OH, —CN, —C₁-C₆ alkyl, —C₁-C₆ halogenalkyl;
two R¹³ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
R¹⁴ and R¹⁵ are each independently selected from H, C₁-C₆ alkyl, cycloalkyl;
or R¹⁴ and R¹⁵ together with the atoms to which they are attached and any intervening atoms, form heterocyclyl optionally substituted with one or more halogen, —OH, —CN, C₁-C₆ alkyl;
each R¹⁶ is independently selected from halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)R¹⁴, —C(O)N—NR¹⁴R¹⁵, cycloalkyl, —O-cycloalkyl, aryl, heterocyclyl, and heteroaryl, wherein the alkyl, alkoxy, cycloalkyl, heterocyclyl, or heteroaryl is optionally substituted with one or more substituents independently selected from halogen, —OH, —CN, C₁-C₆ alkyl, C₁-C₆ halogenalkyl; or
two R¹⁶ together with the atoms to which they are attached and any intervening atoms, form a 5-8 membered cycloalkyl, a 6 membered aryl, a 5-8 membered heterocycle, or a 5-6 membered heteroaryl, wherein cycloalkyl, aryl, heterocycle, or heteroaryl is optionally substituted with one or more substituents independently selected from halogen, —OH, —CN, C₁-C₆ alkyl, C₁-C₆ halogenalkyl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
m is an integer selected from 0 and 1;
n is an integer selected from 0, 1, 2;
x is an integer selected from 0, 1, 2, 3, and 4.

7. The compound of claim 5, wherein the compound is of Formula (I-I-II-VI): 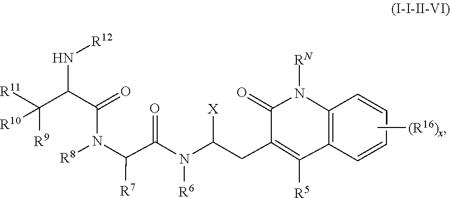
or a pharmaceutically acceptable salt, stereoisomer, solvate, or tautomer thereof.

8. The compound of claim 5, wherein the compound is of Formula (I-II-II-A-A): 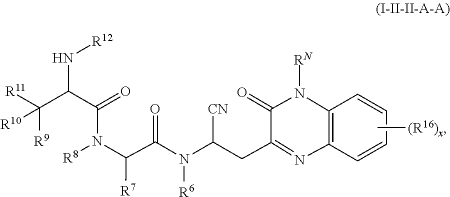
or a pharmaceutically acceptable salt, stereoisomer, solvate, or tautomer thereof.

9. A compound selected from:

| # | Structure | IUPAC Name |
|---|---|---|
| 101 | 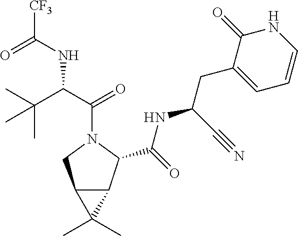 [SMILES: CC(C)(C)[C@H](NC(=O)C(F)(F)F)C(=O)N1C[C@H]2[C@H]([C@H]1C(=O)N[C@H](C#N)Cc1ccc[nH]c1=O)C2(C)C] | (1S,2S,5S)-N-[1-cyano-2-(2-oxo- 1H-pyridin-3-yl)ethyl]-3-[(2S)- 3,3-dimethyl-2-[(2,2,2- trifluoroacetyl)amino]butanoyl]- 6,6-dimethyl-3- azabicyclo[3.1.0]hexane-2- carboxamide |
| 102 | 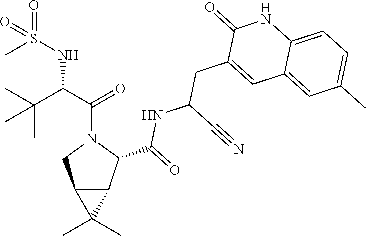 [SMILES: Cc1ccc2[nH]c(=O)c(CC(C#N)NC(=O)[C@@H]3[C@H]4[C@H](CN3C(=O)[C@@H](NS(C)(=O)=O)C(C)(C)C)C4(C)C)cc2c1] | (1S,2S,5S)-N-[1-cyano-2-(6- methyl-2-oxo-1H-quinolin-3- yl)ethyl]-3-[(2S)-2- (methanesulfonamido)-3,3- dimethyl-butanoyl]-6,6-dimethyl- azabicyclo[3.1.0]hexane-2- carboxamide |
| 103 | 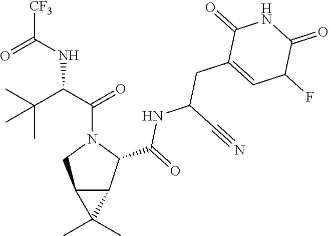 [SMILES: CC(C)(C)[C@H](NC(=O)C(F)(F)F)C(=O)N1C[C@H]2[C@H]([C@H]1C(=O)NC(C#N)CC1=CC(F)C(=O)NC1=O)C2(C)C] | (1S,2S,5S)-N-[1-cyano-2-(3- fluoro-2,6-dioxo-3H-pyridin-5- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 104 | 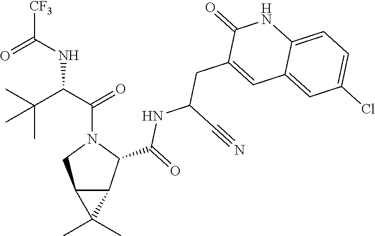 [SMILES: CC(C)(C)[C@H](NC(=O)C(F)(F)F)C(=O)N1C[C@H]2[C@H]([C@H]1C(=O)NC(C#N)Cc1cc3cc(Cl)ccc3[nH]c1=O)C2(C)C] | (1S,2S,5S)-N-[2-(6-chloro-2-oxo- 1H-quinolin-3-yl)-1-cyano-ethyl]- 3-[(2S)-3,3-dimethyl-2-[(2,2,2- trifluoroacetyl)amino]butanoyl]- 6,6-dimethyl-3- azabicyclo[3.1.0]hexane-2- carboxamide |
| 105 | 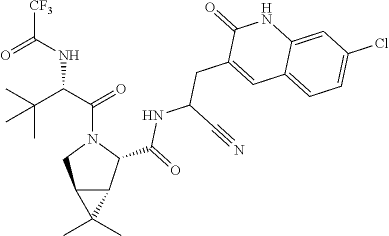 [SMILES: CC(C)(C)[C@H](NC(=O)C(F)(F)F)C(=O)N1C[C@H]2[C@H]([C@H]1C(=O)NC(C#N)Cc1cc3ccc(Cl)cc3[nH]c1=O)C2(C)C] | (1S,2S,5S)-N-[2-(7-chloro-2-oxo- 1H-quinolin-3-yl)-1-cyano-ethyl]- 3-[(2S)-3,3-dimethyl-2-[(2,2,2- trifluoroacetyl)amino]butanoyl]- 6,6-dimethyl-3- azabicyclo[3.1.0]hexane-2- carboxamide |
| 106 | 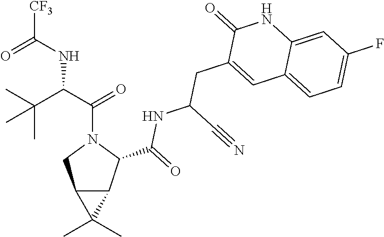 [SMILES: CC(C)(C)[C@H](NC(=O)C(F)(F)F)C(=O)N1C[C@H]2[C@H]([C@H]1C(=O)NC(C#N)Cc1cc3ccc(F)cc3[nH]c1=O)C2(C)C] | (1S,2S,5S)-N-[1-cyano-2-(7- fluoro-2-oxo-1H-quinolin-3- yl)ethyl]-3-[(2S)-3,3-dimethyl-2- [(2,2,2-trifluoroacetyl)amino]butanoyl]- 6,6-dimethyl-3- azabicyclo[3.1.0]hexane-2- carboxamide |
| 107 | 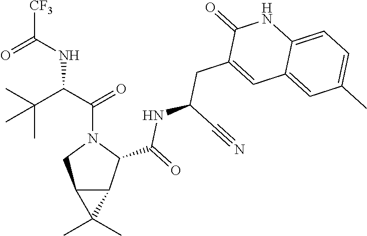 [SMILES: Cc1ccc2[nH]c(=O)c(C[C@@H](C#N)NC(=O)[C@@H]3[C@H]4[C@H](CN3C(=O)[C@@H](NC(=O)C(F)(F)F)C(C)(C)C)C4(C)C)cc2c1] | (1S,2S,5S)-N-[(1S)-1-cyano-2-(6- methyl-2-oxo-1H-quinolin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 108 | 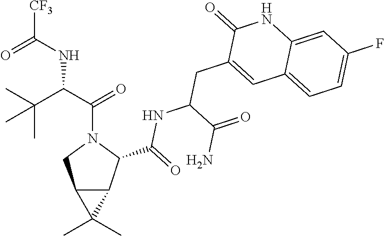 [SMILES: CC(C)(C)[C@H](NC(=O)C(F)(F)F)C(=O)N1C[C@H]2[C@H]([C@H]1C(=O)NC(Cc1cc3ccc(F)cc3[nH]c1=O)C(N)=O)C2(C)C] | (1S,2S,5S)-N-[2-amino-1-[(7- fluoro-2-oxo-1H-quinolin-3- yl)methyl]-2-oxo-ethyl]-3-[(2S)- 3,3-dimethyl-2-[(2,2,2- trifluoroacetyl)amino]butanoyl]- 6,6-dimethyl-3- azabicyclo[3.1.0]hexane-2- carboxamide |
| 109 | 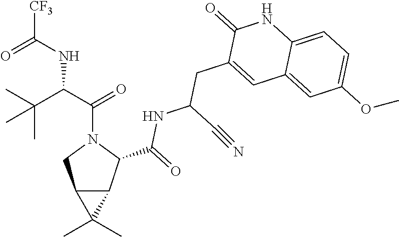 [SMILES: COc1ccc2[nH]c(=O)c(CC(C#N)NC(=O)[C@@H]3[C@H]4[C@H](CN3C(=O)[C@@H](NC(=O)C(F)(F)F)C(C)(C)C)C4(C)C)cc2c1] | (1S,2S,5S)-N-[1-cyano-2-(6- methoxy-2-oxo-1H-quinolin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 110 | 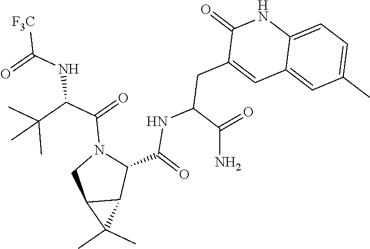 [SMILES: Cc1ccc2[nH]c(=O)c(CC(NC(=O)[C@@H]3[C@H]4[C@H](CN3C(=O)[C@@H](NC(=O)C(F)(F)F)C(C)(C)C)C4(C)C)C(N)=O)cc2c1] | (1S,2S,5S)-N-[2-amino-1-[(6- methyl-2-oxo-1H-quinolin-3- yl)methyl]-2-oxo-ethyl]-3-[(2S)- 3,3-dimethyl-2-[(2,2,2- trifluoroacetyl)amino]butanoyl]- 6,6-dimethyl-3- azabicyclo[3.1.0]hexane-2- carboxamide |
| 111 | 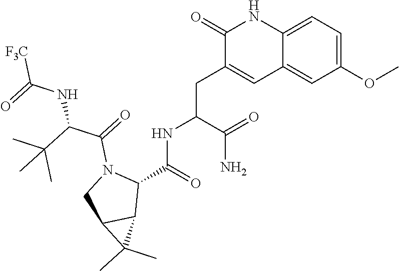 [SMILES: COc1ccc2[nH]c(=O)c(CC(NC(=O)[C@@H]3[C@H]4[C@H](CN3C(=O)[C@@H](NC(=O)C(F)(F)F)C(C)(C)C)C4(C)C)C(N)=O)cc2c1] | (1S,2S,5S)-N-[2-amino-1-[(6- methoxy-2-oxo-1H-quinolin-3- yl)methyl]-2-oxo-ethyl]-3-[(2S)- 3,3-dimethyl-2-[(2,2,2- trifluoroacetyl)amino]butanoyl]- 6,6-dimethyl-3- azabicyclo[3.1.0]hexane-2- carboxamide |
| 112 | 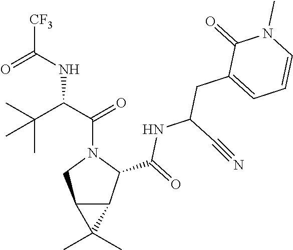 [SMILES: Cn1cccc(CC(C#N)NC(=O)[C@@H]2[C@H]3[C@H](CN2C(=O)[C@@H](NC(=O)C(F)(F)F)C(C)(C)C)C3(C)C)c1=O] | (1S,2S,5S)-N-[1-cyano-2-(1- methyl-2-oxo-3-pyridyl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 113 | 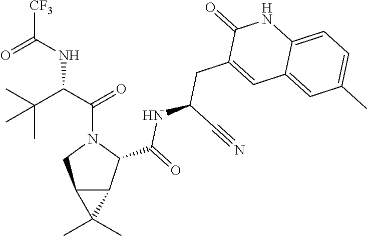 [SMILES: Cc1ccc2[nH]c(=O)c(C[C@@H](C#N)NC(=O)[C@@H]3[C@H]4[C@H](CN3C(=O)[C@@H](NC(=O)C(F)(F)F)C(C)(C)C)C4(C)C)cc2c1] | (1S,2S,5S)-N-[(1S)-1-cyano-2-(6- methyl-2-oxo-1H-quinolin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 114 | 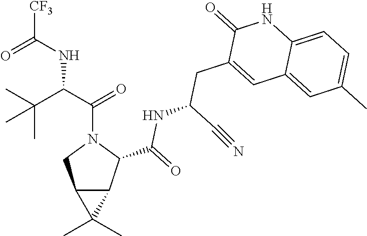 [SMILES: Cc1ccc2[nH]c(=O)c(C[C@H](C#N)NC(=O)[C@@H]3[C@H]4[C@H](CN3C(=O)[C@@H](NC(=O)C(F)(F)F)C(C)(C)C)C4(C)C)cc2c1] | (1S,2S,5S)-N-[(1R)-1-cyano-2- (6-methyl-2-oxo-1H-quinolin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 115 | 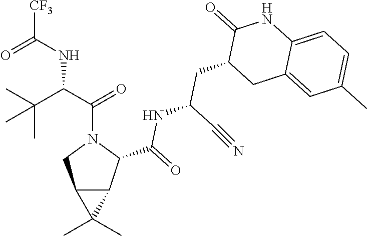 [SMILES: Cc1ccc2c(c1)C[C@H](C[C@H](C#N)NC(=O)[C@@H]1[C@H]3[C@H](CN1C(=O)[C@@H](NC(=O)C(F)(F)F)C(C)(C)C)C3(C)C)C(=O)N2] | (1S,2S,5S)-N-[(1R)-1-cyano-2- [(3R)-6-methyl-2-oxo-3,4- dihydro-1H-quinolin-3-yl]ethyl]- 3-[(2S)-3,3-dimethyl-2-[(2,2,2- trifluoroacetyl)amino]butanoyl]- 6,6-dimethyl-3- azabicyclo[3.1.0]hexane-2- carboxamide |
| 116 | 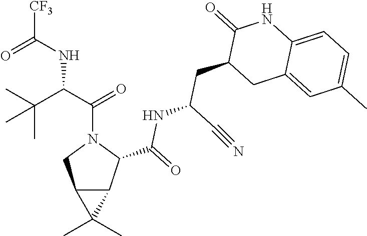 [SMILES: Cc1ccc2c(c1)C[C@@H](C[C@H](C#N)NC(=O)[C@@H]1[C@H]3[C@H](CN1C(=O)[C@@H](NC(=O)C(F)(F)F)C(C)(C)C)C3(C)C)C(=O)N2] | (1S,2S,5S)-N-[(1R)-1-cyano-2- [(3S)-6-methyl-2-oxo-3,4- dihydro-1H-quinolin-3-yl]ethyl]- 3-[(2S)-3,3-dimethyl-2-[(2,2,2- trifluoroacetyl)amino]butanoyl]- 6,6-dimethyl-3- azabicyclo[3.1.0]hexane-2- carboxamide |
| 117 | 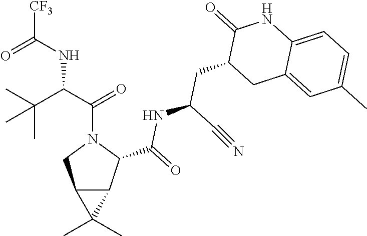 [SMILES: Cc1ccc2c(c1)C[C@H](C[C@@H](C#N)NC(=O)[C@@H]1[C@H]3[C@H](CN1C(=O)[C@@H](NC(=O)C(F)(F)F)C(C)(C)C)C3(C)C)C(=O)N2] | (1S,2S,5S)-N-[(1S)-1-cyano-2- [(3R)-6-methyl-2-oxo-3,4- dihydro-1H-quinolin-3-yl]ethyl]- 3-[(2S)-3,3-dimethyl-2-[(2,2,2- trifluoroacetyl)amino]butanoyl]- 6,6-dimethyl-3- azabicyclo[3.1.0]hexane-2- carboxamide |
| 118 | 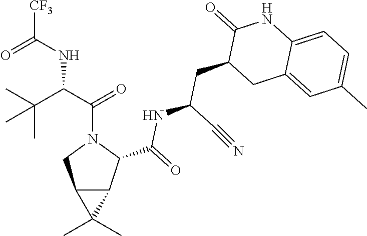 [SMILES: Cc1ccc2c(c1)C[C@@H](C[C@@H](C#N)NC(=O)[C@@H]1[C@H]3[C@H](CN1C(=O)[C@@H](NC(=O)C(F)(F)F)C(C)(C)C)C3(C)C)C(=O)N2] | (1S,2S,5S)-N-[(1S)-1-cyano-2- [(3S)-6-methyl-2-oxo-3,4- dihydro-1H-quinolin-3-yl]ethyl]- 3-[(2S)-3,3-dimethyl-2-[(2,2,2- trifluoroacetyl)amino]butanoyl]- 6,6-dimethyl-3- azabicyclo[3.1.0]hexane-2- carboxamide |
| 119 | 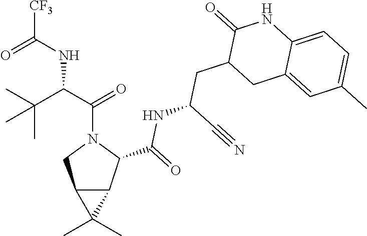 [SMILES: Cc1ccc2c(c1)CC(C[C@H](C#N)NC(=O)[C@@H]1[C@H]3[C@H](CN1C(=O)[C@@H](NC(=O)C(F)(F)F)C(C)(C)C)C3(C)C)C(=O)N2] | (1S,2S,5S)-N-[(1R)-1-cyano-2- (6-methyl-2-oxo-3,4-dihydro-1H- quinolin-3-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 120 | 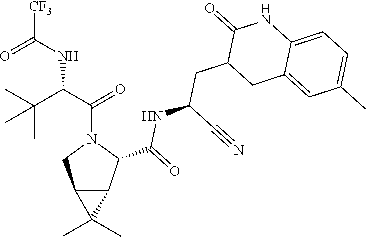 [SMILES: Cc1ccc2c(c1)CC(C[C@@H](C#N)NC(=O)[C@@H]1[C@H]3[C@H](CN1C(=O)[C@@H](NC(=O)C(F)(F)F)C(C)(C)C)C3(C)C)C(=O)N2] | (1S,2S,5S)-N-[(1S)-1-cyano-2-(6- methyl-2-oxo-3,4-dihydro-1H- quinolin-3-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 121 | 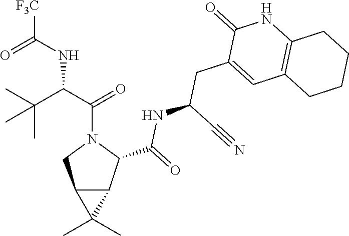 [SMILES: CC(C)(C)[C@H](NC(=O)C(F)(F)F)C(=O)N1C[C@H]2[C@H]([C@H]1C(=O)N[C@H](C#N)Cc1cc3c([nH]c1=O)CCCC3)C2(C)C] | (1S,2S,5S)-N-[(1-cyano-2-(2-oxo- 5,6,7,8-tetrahydro-1H-quinolin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 122 | 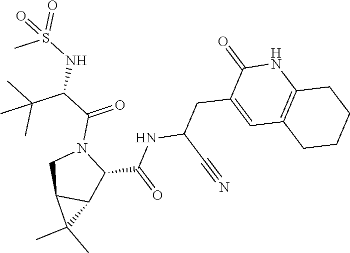 [SMILES: CC(C)(C)[C@H](NS(C)(=O)=O)C(=O)N1C[C@H]2[C@H]([C@H]1C(=O)NC(C#N)Cc1cc3c([nH]c1=O)CCCC3)C2(C)C] | (1S,2S,5S)-N-[1-cyano-2-(2-oxo- 5,6,7,8-tetrahydro-1H-quinolin-3- yl)ethyl]-3-[(2S)-2- (methanesulfonamido)-3,3- dimethyl-butanoyl]-6,6-dimethyl- 3-azabicyclo[3.1.0]hexane-2- carboxamide |
| 123 | 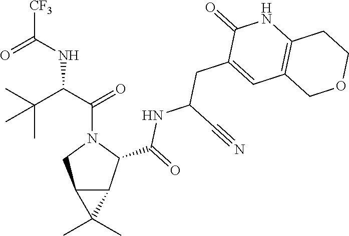 [SMILES: CC(C)(C)[C@H](NC(=O)C(F)(F)F)C(=O)N1C[C@H]2[C@H]([C@H]1C(=O)NC(C#N)Cc1cc3c([nH]c1=O)CCOC3)C2(C)C] | (1S,2S,5S)-N-[1-cyano-2-(2-oxo- 1,5,7,8-tetrahydropyran[4,3- b]pyridin-3-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 124 | 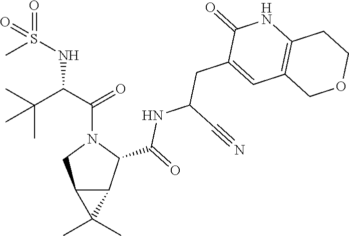 [SMILES: CC(C)(C)[C@H](NS(C)(=O)=O)C(=O)N1C[C@H]2[C@H]([C@H]1C(=O)NC(C#N)Cc1cc3c([nH]c1=O)CCOC3)C2(C)C] | (1S,2S,5S)-N-[1-cyano-2-(2-oxo- 1,5,7,8-tetrahydropyrano[4,3- b]pyridin-3-yl)ethyl]-3-[(2S)-2- (methanesulfonamido)-3,3- dimethyl-butanoyl]-6,6-dimethyl-3- azabicyclo[3.1.0]hexane-2- carboxamide |
| 125 | 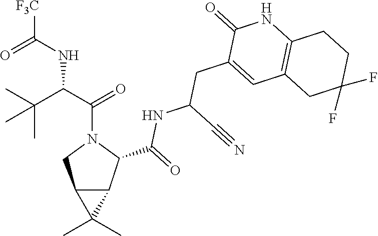 [SMILES: CC(C)(C)[C@H](NC(=O)C(F)(F)F)C(=O)N1C[C@H]2[C@H]([C@H]1C(=O)NC(C#N)Cc1cc3c([nH]c1=O)CCC(F)(F)C3)C2(C)C] | (1S,2S,5S)-N-[1-cyano-2-(6,6- difluoro-2-oxo-1,5,7,8- tetrahydroquinolin-3-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 126 | 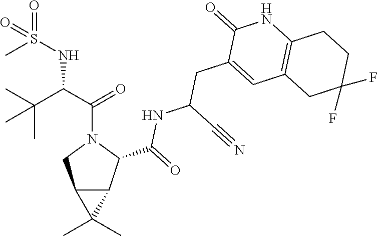 [SMILES: CC(C)(C)[C@H](NS(C)(=O)=O)C(=O)N1C[C@H]2[C@H]([C@H]1C(=O)NC(C#N)Cc1cc3c([nH]c1=O)CCC(F)(F)C3)C2(C)C] | (1S,2S,5S)-N-[1-cyano-2-(6,6- difluoro-2-oxo-1,5,7,8- tetrahydroquinolin-3-yl)ethyl]-3- [(2S)-2-(methanesulfonamido)- 3,3-dimethyl-butanoyl]-6,6- dimethyl-3- azabicyclo[3.1.0]hexane-2- carboxamide |
| 127 | 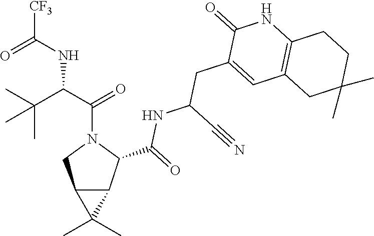 [SMILES: CC1(C)CCc2[nH]c(=O)c(CC(C#N)NC(=O)[C@@H]3[C@H]4[C@H](CN3C(=O)[C@@H](NC(=O)C(F)(F)F)C(C)(C)C)C4(C)C)cc2C1] | (1S,2S,5S)-N-[1-cyano-2-(6,6- dimethyl-2-oxo-1,5,7,8- tetrahydroquinolin-3-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 128 | 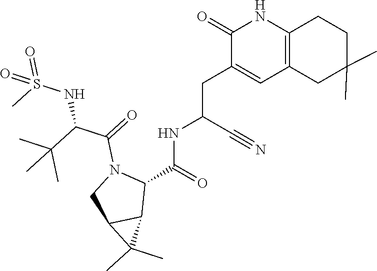 [SMILES: CC1(C)CCc2[nH]c(=O)c(CC(C#N)NC(=O)[C@@H]3[C@H]4[C@H](CN3C(=O)[C@@H](NS(C)(=O)=O)C(C)(C)C)C4(C)C)cc2C1] | (1S,2S,5S)-N-[1-cyano-2-(6,6- dimethyl-2-oxo-1,5,7,8- tetrahydroquinolin-3-yl)ethyl]-3- [(2S)-2-(methanesulfonamido)- 3,3-dimethyl-butanoyl]-6,6- dimethyl-3- azabicyclo[3.1.0]hexane-2- carboxamide |
| 129 | 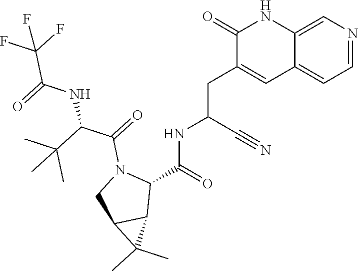 [SMILES: CC(C)(C)[C@H](NC(=O)C(F)(F)F)C(=O)N1C[C@H]2[C@H]([C@H]1C(=O)NC(C#N)Cc1cc3ccncc3[nH]c1=O)C2(C)C] | (1S,2S,5S)-N-[1-cyano-2-(2-oxo- 1H-1,6-naphthyridin-3-yl)ethyl]- 3-[(2S)-3,3-dimethyl-2-[(2,2,2- trifluoroacetyl)amino]butanoyl]- 6,6-dimethyl-3- azabicyclo[3.1.0]hexane-2- carboxamide |
| 130 | 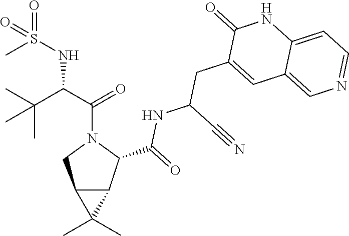 [SMILES: CC(C)(C)[C@H](NS(C)(=O)=O)C(=O)N1C[C@H]2[C@H]([C@H]1C(=O)NC(C#N)Cc1cc3cnccc3[nH]c1=O)C2(C)C] | (1S,2S,5S)-N-[1-cyano-2-(2-oxo- 1H-1,6-naphthyridin-3-yl)ethyl]- 3-[(2S)-2-(methanesulfonamido)- 3,3-dimethyl-butanoyl]- 6,6-dimethyl-3- azabicyclo[3.1.0]hexane-2- carboxamide |
| 131 | 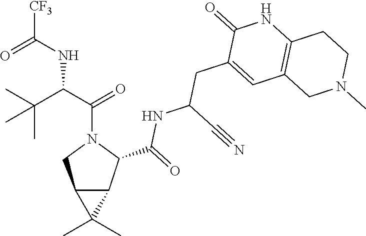 [SMILES: CN1CCc2[nH]c(=O)c(CC(C#N)NC(=O)[C@@H]3[C@H]4[C@H](CN3C(=O)[C@@H](NC(=O)C(F)(F)F)C(C)(C)C)C4(C)C)cc2C1] | (1S,2S,5S)-N-[1-cyano-2-(6- methyl-2-oxo-1,5,7,8-tetrahydro- 1,6-naphthyridin-3-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 132 | 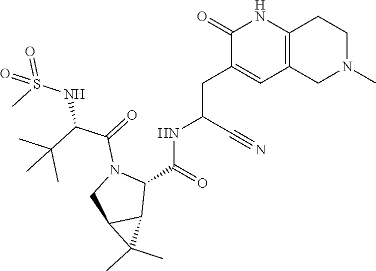 [SMILES: CN1CCc2[nH]c(=O)c(CC(C#N)NC(=O)[C@@H]3[C@H]4[C@H](CN3C(=O)[C@@H](NS(C)(=O)=O)C(C)(C)C)C4(C)C)cc2C1] | (1R,2S,5S)-N-[1-cyano-2-(6- methyl-2-oxo-1,5,7,8-tetrahydro- 1,6-naphthyridin-3-yl)ethyl]-3- [(2S)-2-(methanesulfonamido)- 3,3-dimethyl-butanoyl]- 6,6-dimethyl-3- azabicyclo[3.1.0]hexane-2- carboxamide |
| 133 | 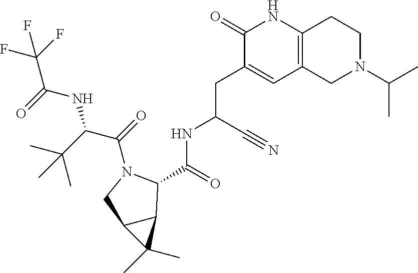 [SMILES: CC(C)N1CCc2[nH]c(=O)c(CC(C#N)NC(=O)[C@@H]3[C@@H]4[C@H](CN3C(=O)[C@@H](NC(=O)C(F)(F)F)C(C)(C)C)C4(C)C)cc2C1] | (1R,2S,5S)-N-[1-cyano-2-(6- isopropyl-2-oxo-1,5,7,8- tetrahydro-1,6-naphthyridin-3- yl)ethyl]-3-[(2S)-3,3-dimethyl-2- [(2,2,2-trifluoroacetyl)amino] butanoyl]-6,6-dimethyl-3- azabicyclo[3.1.0]hexane-2- carboxamide |
| 134 | 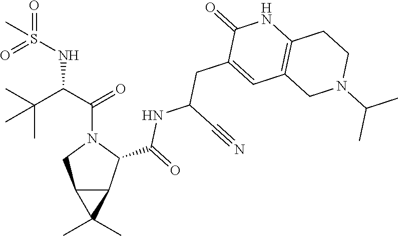 [SMILES: CC(C)N1CCc2[nH]c(=O)c(CC(C#N)NC(=O)[C@@H]3[C@@H]4[C@H](CN3C(=O)[C@@H](NS(C)(=O)=O)C(C)(C)C)C4(C)C)cc2C1] | (1R,2S,5S)-N-[1-cyano-2-(6- isopropyl-2-oxo-1,5,7,8- tetrahydro-1,6-naphthyridin-3- yl)ethyl]-3-[(2S)-2- (methanesulfonamido)-3,3- dimethyl-butanoyl]-6,6-dimethyl-3- azabicyclo[3.1.0]hexane-2- carboxamide |
| 135 | 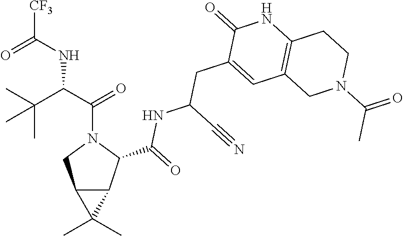 [SMILES: CC(=O)N1CCc2[nH]c(=O)c(CC(C#N)NC(=O)[C@@H]3[C@H]4[C@H](CN3C(=O)[C@@H](NC(=O)C(F)(F)F)C(C)(C)C)C4(C)C)cc2C1] | (1S,2S,5S)-N-[2-(6-acetyl-2-oxo- 1,5,7,8-tetrahydro-1,6- naphthyridin-3-yl)-1-cyano- ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 136 | 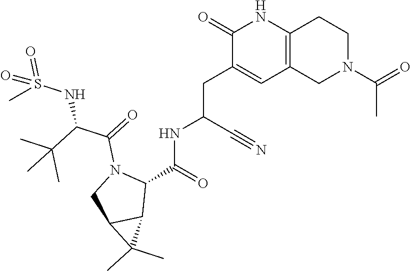 [SMILES: CC(=O)N1CCc2[nH]c(=O)c(CC(C#N)NC(=O)[C@@H]3[C@H]4[C@H](CN3C(=O)[C@@H](NS(C)(=O)=O)C(C)(C)C)C4(C)C)cc2C1] | (1S,2S,5S)-N-[2-(6-acetyl-2-oxo- 1,5,7,8-tetrahydro-1,6- naphthyridin-3-yl)-1-cyano- ethyl]-3-[(2S)-2- (methanesulfonamido)-3,3- dimethyl-butanoyl]-6,6-dimethyl-3- azabicyclo[3.1.0]hexane-2- carboxamide |
| 137 | 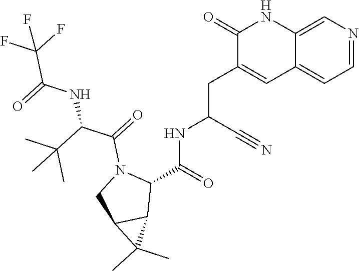 [SMILES: CC(C)(C)[C@H](NC(=O)C(F)(F)F)C(=O)N1C[C@H]2[C@H]([C@H]1C(=O)NC(C#N)Cc1cc3ccncc3[nH]c1=O)C2(C)C] | (1S,2S,5S)-N-[1-cyano-2-(2-oxo- 1H-1,7-naphthyridin-3-yl)ethyl]- 3-[(2S)-3,3-dimethyl-2-[(2,2,2- trifluoroacetyl)amino]butanoyl]- 6,6-dimethyl-3- azabicyclo[3.1.0]hexane-2- carboxamide |
| 138 | 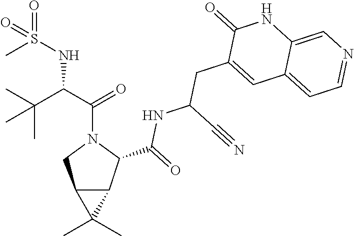 [SMILES: CC(C)(C)[C@H](NS(C)(=O)=O)C(=O)N1C[C@H]2[C@H]([C@H]1C(=O)NC(C#N)Cc1cc3ccncc3[nH]c1=O)C2(C)C] | (1S,2S,5S)-N-[1-cyano-2-(2-oxo- 1H-1,7-naphthyridin-3-yl)ethyl]- 3-[(2S)-2-(methanesulfonamido)- 3,3-dimethyl-butanoyl]-6,6- dimethyl-3- azabicyclo[3.1.0]hexane-2- carboxamide |
| 139 | 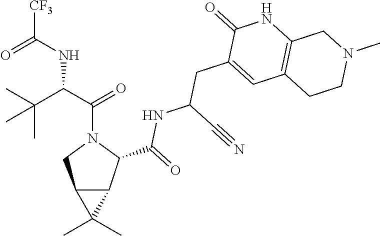 [SMILES: CN1CCc2cc(CC(C#N)NC(=O)[C@@H]3[C@H]4[C@H](CN3C(=O)[C@@H](NC(=O)C(F)(F)F)C(C)(C)C)C4(C)C)c(=O)[nH]c2C1] | (1S,2S,5S)-N-[1-cyano-2-(7- methyl-2-oxo-1,5,6,8-tetrahydro- 1,7-naphthyridin-3-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 140 | 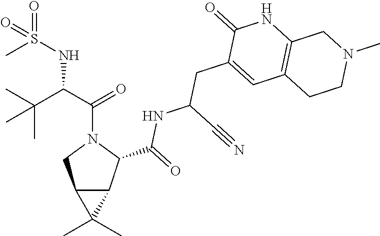 [SMILES: CN1CCc2cc(CC(C#N)NC(=O)[C@@H]3[C@H]4[C@H](CN3C(=O)[C@@H](NS(C)(=O)=O)C(C)(C)C)C4(C)C)c(=O)[nH]c2C1] | (1S,2S,5S)-N-[1-cyano-2-(7- methyl-2-oxo-1,5,6,8-tetrahydro- 1,7-naphthyridin-3-yl)ethyl]-3- [(2S)-2-(methanesulfonamido)- 3,3-dimethyl-butanoyl]- 6,6-dimethyl-3- azabicyclo[3.1.0]hexane-2- carboxamide |
| 141 | 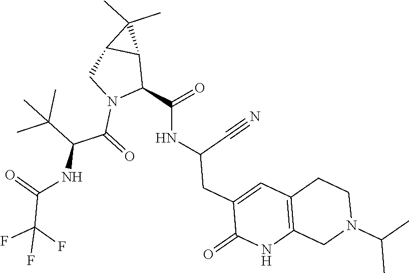 [SMILES: CC(C)N1CCc2cc(CC(C#N)NC(=O)[C@@H]3[C@@H]4[C@H](CN3C(=O)[C@@H](NC(=O)C(F)(F)F)C(C)(C)C)C4(C)C)c(=O)[nH]c2C1] | (1R,2S,5S)-N-[1-cyano-2-(7- isopropyl-2-oxo-1,5,6,8- tetrahydro-1,7-naphthyridin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 142 | 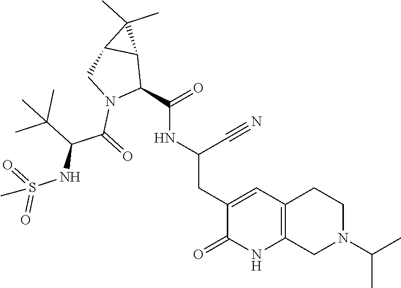 [SMILES: CC(C)N1CCc2cc(CC(C#N)NC(=O)[C@@H]3[C@@H]4[C@H](CN3C(=O)[C@@H](NS(C)(=O)=O)C(C)(C)C)C4(C)C)c(=O)[nH]c2C1] | (1R,2S,5S)-N-[1-cyano-2-(7- isopropyl-2-oxo-1,5,6,8- tetrahydro-1,7-naphthyridin-3- yl)ethyl]-3-[(2S)-2- (methanesulfonamido)-3,3- dimethyl-butanoyl]-6,6-dimethyl-3- azabicyclo[3.1.0]hexane-2- carboxamide |
| 143 | 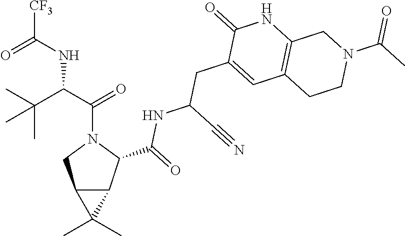 [SMILES: CC(=O)N1CCc2cc(CC(C#N)NC(=O)[C@@H]3[C@H]4[C@H](CN3C(=O)[C@@H](NC(=O)C(F)(F)F)C(C)(C)C)C4(C)C)c(=O)[nH]c2C1] | (1S,2S,5S)-N-[2-(7-acetyl-2-oxo- 1,5,6,8-tetrahydro-1,7- naphthyridin-3-yl)-1-cyano- ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 144 | 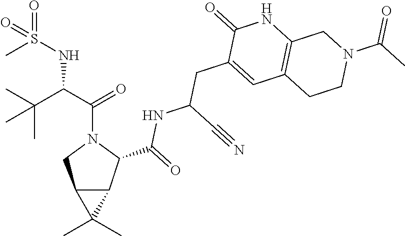 [SMILES: CC(=O)N1CCc2cc(CC(C#N)NC(=O)[C@@H]3[C@H]4[C@H](CN3C(=O)[C@@H](NS(C)(=O)=O)C(C)(C)C)C4(C)C)c(=O)[nH]c2C1] | (1S,2S,5S)-N-[2-(7-acetyl-2-oxo- 1,5,6,8-tetrahydro-1,7- naphthyridin-3-yl)-1-cyano- ethyl]-3-[(2S)-2- (methanesulfonamido)-3,3- dimethyl-butanoyl]- 6,6-dimethyl-3- azabicyclo[3.1.0]hexane-2- carboxamide |
| 145 | 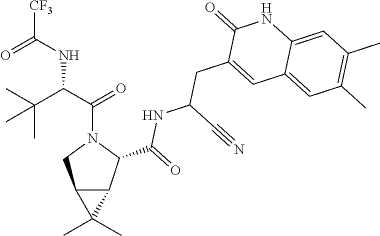 [SMILES: Cc1cc2cc(CC(C#N)NC(=O)[C@@H]3[C@H]4[C@H](CN3C(=O)[C@@H](NC(=O)C(F)(F)F)C(C)(C)C)C4(C)C)c(=O)[nH]c2cc1C] | (1S,2S,5S)-N-[1-cyano-2-(6,7- dimethyl-2-oxo-1H-quinolin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 146 | 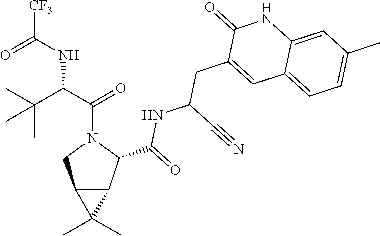 [SMILES: Cc1ccc2cc(CC(C#N)NC(=O)[C@@H]3[C@H]4[C@H](CN3C(=O)[C@@H](NC(=O)C(F)(F)F)C(C)(C)C)C4(C)C)c(=O)[nH]c2c1] | (1S,2S,5S)-N-[1-cyano-2-(7- methyl-2-oxo-1H-quinolin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 147 | 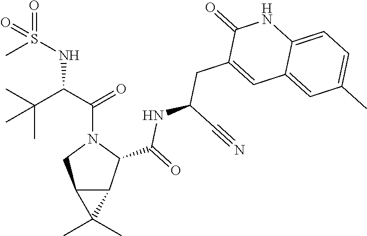 [SMILES: Cc1ccc2[nH]c(=O)c(C[C@@H](C#N)NC(=O)[C@@H]3[C@H]4[C@H](CN3C(=O)[C@@H](NS(C)(=O)=O)C(C)(C)C)C4(C)C)cc2c1] | (1S,2S,5S)-N-[(1S)-1-cyano-2-(6- methyl-2-oxo-1H-quinolin-3- yl)ethyl]-3-[(2S)-2- (methanesulfonamido)-3,3- dimethyl-butanoyl]-6,6-dimethyl- 3-azabicyclo[3.1.0]hexane-2- carboxamide |
| 148 | 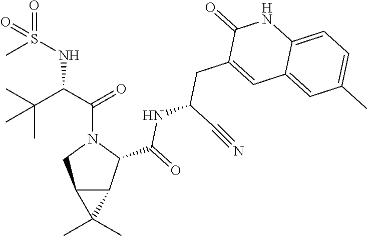 [SMILES: Cc1ccc2[nH]c(=O)c(C[C@H](C#N)NC(=O)[C@@H]3[C@H]4[C@H](CN3C(=O)[C@@H](NS(C)(=O)=O)C(C)(C)C)C4(C)C)cc2c1] | (1S,2S,5S)-N-[(1R)-1-cyano-2- (6-methyl-2-oxo-1H-quinolin-3- yl)ethyl]-3-[(2S)-2- (methanesulfonamido)-3,3- dimethyl-butanoyl]-6,6-dimethyl- 3-azabicyclo[3.1.0]hexane-2- carboxamide |
| 149 | 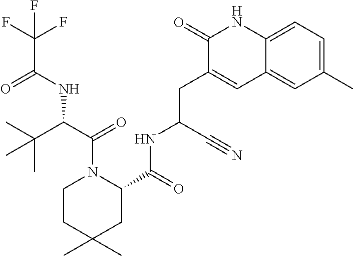 [SMILES: Cc1ccc2[nH]c(=O)c(CC(C#N)NC(=O)[C@@H]3CC(C)(C)CCN3C(=O)[C@@H](NC(=O)C(F)(F)F)C(C)(C)C)cc2c1] | (2S)-N-[1-cyano-2-(6-methyl-2- oxo-1H-quinolin-3-yl)ethyl]-1- [(2S)-3,3-dimethyl-2-[(2,2,2- trifluoroacetyl)amino]butanoyl]- 4,4-dimethyl-piperidine-2- carboxamide |
| 150 | 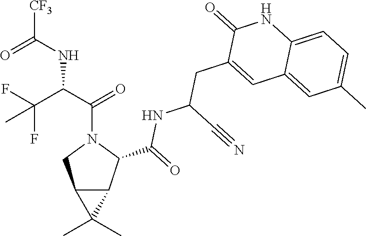 [SMILES: Cc1ccc2[nH]c(=O)c(CC(C#N)NC(=O)[C@@H]3[C@H]4[C@H](CN3C(=O)[C@@H](NC(=O)C(F)(F)F)C(C)(F)F)C4(C)C)cc2c1] | (1S,2S,5S)-N-[1-cyano-2-(6- methyl-2-oxo-1H-quinolin-3- yl)ethyl]-3-[(2R)-3,3-difluoro-2- [(2,2,2- trifluoroacetyl)amino]butanoyl]- 6,6-dimethyl-3- azabicyclo[3.1.0]hexane-2- carboxamide |
| 151 | 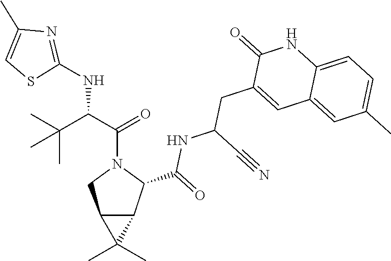 | (1S,2S,5S)-N-[1-cyano-2-(6- methyl-2-oxo-1H-quinolin-3- yl)ethyl]-3-[(2S)-3,3-dimethyl-2- [(4-methylthiazol-2- yl)amino]butanoyl]-6,6-dimethyl- 3-azabicyclo[3.1.0]hexane-2- carboxamide |
| 152 | 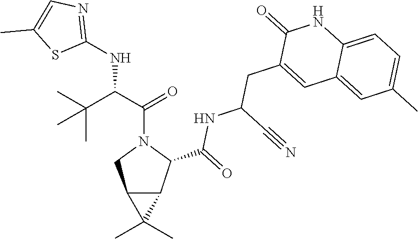 | (1S,2S,5S)-N-[1-cyano-2-(6- methyl-2-oxo-1H-quinolin-3- yl)ethyl]-3-[(2S)-3,3-dimethyl-2- [(5-methylthiazol-2- yl)amino]butanoyl]-6,6-dimethyl- 3-azabicyclo[3.1.0]hexane-2- carboxamide |
| 153 | 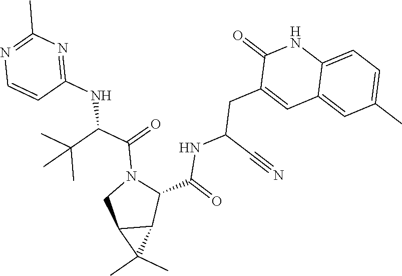 [SMILES: Cc1ccc2[nH]c(=O)c(CC(C#N)NC(=O)[C@@H]3[C@H]4[C@H](CN3C(=O)[C@@H](Nc3ccnc(C)n3)C(C)(C)C)C4(C)C)cc2c1] | (1S,2S,5S)-N-[1-cyano-2-(6- methyl-2-oxo-1H-quinolin-3- yl)ethyl]-3-[(2S)-3,3-dimethyl-2- [(2-methylpyrimidin-4- yl)amino]butanoyl]-6,6-dimethyl- 3-azabicyclo[3.1.0]hexane-2- carboxamide |
| 154 | 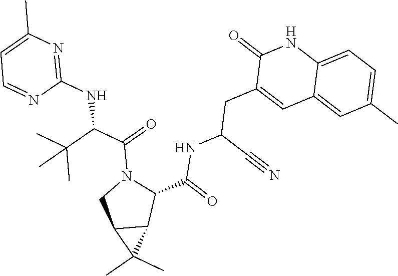 [SMILES: Cc1ccc2[nH]c(=O)c(CC(C#N)NC(=O)[C@@H]3[C@H]4[C@H](CN3C(=O)[C@@H](Nc3nccc(C)n3)C(C)(C)C)C4(C)C)cc2c1] | (1S,2S,5S)-N-[1-cyano-2-(6- methyl-2-oxo-1H-quinolin-3- yl)ethyl]-3-[(2S)-3,3-dimethyl-2- [(4-methylpyrimidin-2- yl)amino]butanoyl-6,6-dimethyl- 3-azabicyclo[3.1.0]hexane-2- carboxamide |
| 155 | 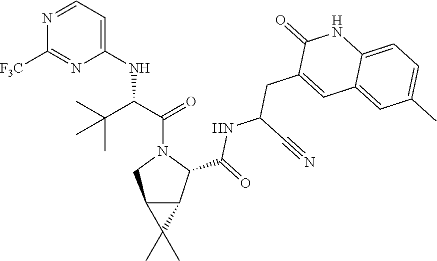 [SMILES: Cc1ccc2[nH]c(=O)c(CC(C#N)NC(=O)[C@@H]3[C@H]4[C@H](CN3C(=O)[C@@H](Nc3ccnc(C(F)(F)F)n3)C(C)(C)C)C4(C)C)cc2c1] | (1S,2S,5S)-N-[1-cyano-2-(6- methyl-2-oxo-1H-quinolin-3- yl)ethyl]-3-[(2S)-3,3-dimethyl-2- [[2-(trifluoromethyl)pyrimidin-4- yl]amino]butanoyl]-6,6-dimethyl- 3-azabicyclo[3.1.0]hexane-2- carboxamide |
| 156 | 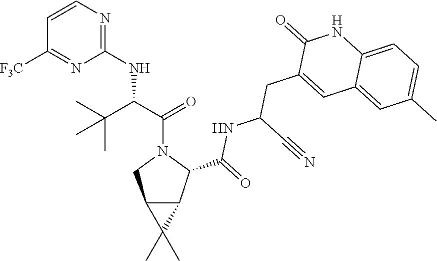 [SMILES: Cc1ccc2[nH]c(=O)c(CC(C#N)NC(=O)[C@@H]3[C@H]4[C@H](CN3C(=O)[C@@H](Nc3nccc(C(F)(F)F)n3)C(C)(C)C)C4(C)C)cc2c1] | (1S,2S,5S)-N-[1-cyano-2-(6- methyl-2-oxo-1H-quinolin-3- yl)ethyl]-3-[(2S)-3,3-dimethyl-2- [[4-(trifluoromethyl)pyrimidin-2- yl]amino]butanoyl]-6,6-dimethyl- 3-azabicyclo[3.1.0]hexane-2- carboxamide |
| 157 | 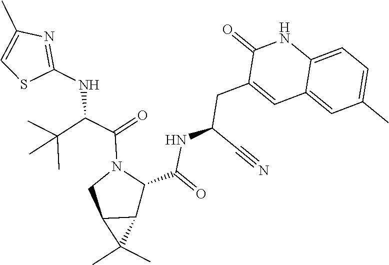 [SMILES: Cc1ccc2[nH]c(=O)c(C[C@@H](C#N)NC(=O)[C@@H]3[C@H]4[C@H](CN3C(=O)[C@@H](Nc3nc(C)cs3)C(C)(C)C)C4(C)C)cc2c1] | (1S,2S,5S)-N-[(1S)-1-cyano-2-(6- methyl-2-oxo-1H-quinolin-3- yl)ethyl]-3-[(2S)-3,3-dimethyl-2- [(4-methylthiazol-2- yl)amino]butanoyl]-6,6-dimethyl- 3-azabicyclo[3.1.0]hexane-2- carboxamide |
| 158 | 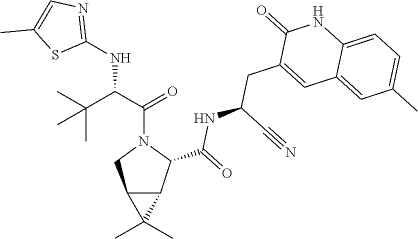 | (1S,2S,5S)-N-[(1S)-1-cyano-2-(6- methyl-2-oxo-1H-quinolin-3- yl)ethyl]-3-[(2S)-3,3-dimethyl-2- [(5-methylthiazol-2- yl)amino]butanoyl]-6,6-dimethyl- 3-azabicyclo[3.1.0]hexane-2- carboxamide |
| 159 | 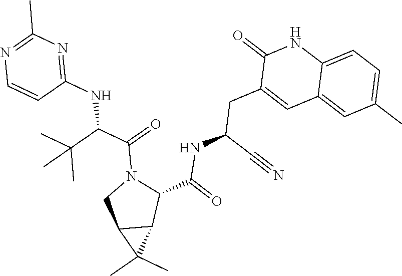 [SMILES: Cc1ccc2[nH]c(=O)c(C[C@@H](C#N)NC(=O)[C@@H]3[C@H]4[C@H](CN3C(=O)[C@@H](Nc3ccnc(C)n3)C(C)(C)C)C4(C)C)cc2c1] | (1S,2S,5S)-N-[(1S)-1-cyano-2-(6- methyl-2-oxo-1H-quinolin-3- yl)ethyl]-3-[(2S)-3,3-dimethyl-2- [(2-methylpyrimidin-4- yl)amino]butanoyl]-6,6-dimethyl- 3-azabicyclo[3.1.0]hexane-2- carboxamide |
| 160 | 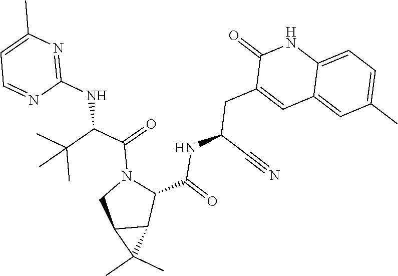 [SMILES: Cc1ccc2[nH]c(=O)c(C[C@@H](C#N)NC(=O)[C@@H]3[C@H]4[C@H](CN3C(=O)[C@@H](Nc3nccc(C)n3)C(C)(C)C)C4(C)C)cc2c1] | (1S,2S,5S)-N-[(1S)-1-cyano-2-(6- methyl-2-oxo-1H-quinolin-3- yl)ethyl]-3-[(2S)-3,3-dimethyl-2- [(4-methylpyrimidin-2- yl)amino]butanoyl]-6,6-dimethyl- 3-azabicyclo[3.1.0]hexane-2- carboxamide |
| 161 | 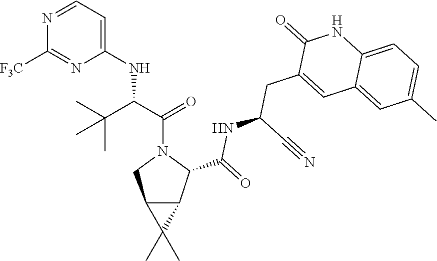 [SMILES: Cc1ccc2[nH]c(=O)c(C[C@@H](C#N)NC(=O)[C@@H]3[C@H]4[C@H](CN3C(=O)[C@@H](Nc3ccnc(C(F)(F)F)n3)C(C)(C)C)C4(C)C)cc2c1] | (1S,2S,5S)-N-[(1S)-1-cyano-2-(6- methyl-2-oxo-1H-quinolin-3- yl)ethyl]-3-[(2S)-3,3-dimethyl-2- [[2-(trifluoromethyl)pyrimidin-4- yl]amino]butanoyl]-6,6-dimethyl- 3-azabicyclo[3.1.0]hexane-2- carboxamide |
| 162 | 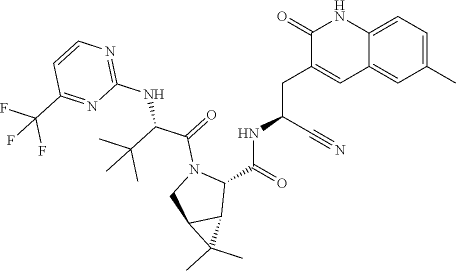 [SMILES: Cc1ccc2[nH]c(=O)c(C[C@@H](C#N)NC(=O)[C@@H]3[C@H]4[C@H](CN3C(=O)[C@@H](Nc3nccc(C(F)(F)F)n3)C(C)(C)C)C4(C)C)cc2c1] | (1S,2S,5S)-N-[(1S)-1-cyano-2-(6- methyl-2-oxo-1H-quinolin-3- yl)ethyl]-3-[(2S)-3,3-dimethyl-2- [[4-(trifluoromethyl)pyrimidin-2- yl]amino]butanoyl]-6,6-dimethyl- 3-azabicyclo[3.1.0]hexane-2- carboxamide |
| 163 | 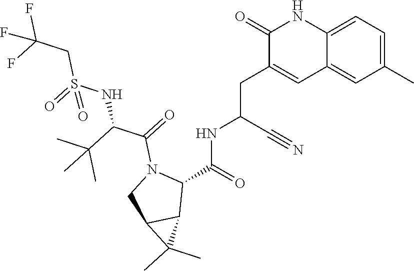 [SMILES: Cc1ccc2[nH]c(=O)c(CC(C#N)NC(=O)[C@@H]3[C@H]4[C@H](CN3C(=O)[C@@H](NS(=O)(=O)CC(F)(F)F)C(C)(C)C)C4(C)C)cc2c1] | (1S,2S,5S)-N-[1-cyano-2-(6- methyl-2-oxo-1H-quinolin-3- yl)ethyl]-3-[(2S)-3,3-dimethyl-2- (2,2,2-trifluoroethylsulfonylamino) butanoyl]-6,6-dimethyl-3- azabicyclo[3.1.0]hexane-2- carboxamide |
| 164 | 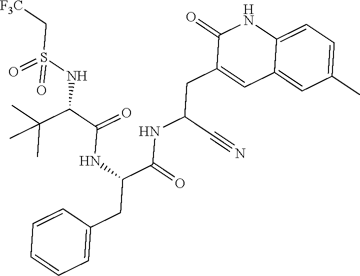 [SMILES: Cc1ccc2[nH]c(=O)c(CC(C#N)NC(=O)[C@H](Cc3ccccc3)NC(=O)[C@@H](NS(=O)(=O)CC(F)(F)F)C(C)(C)C)cc2c1] | (2S)-N-[(1S)-1-benzyl-2-[[1- cyano-2-(6-methyl-2-oxo-1H- quinolin-3-yl)ethyl]amino]-2- oxo-ethyl]-3,3-dimethyl-2-(2,2,2- trifluoroethylsulfonylamino) butanamide |
| 165 | 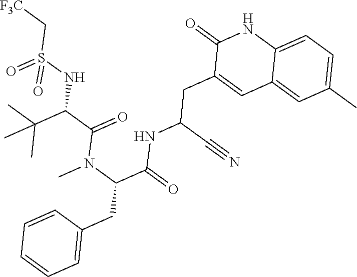 [SMILES: Cc1ccc2[nH]c(=O)c(CC(C#N)NC(=O)[C@H](Cc3ccccc3)N(C)C(=O)[C@@H](NS(=O)(=O)CC(F)(F)F)C(C)(C)C)cc2c1] | (2S)-N-[(1S)-1-benzyl-2-[[1- cyano-2-(6-methyl-2-oxo-1H- quinolin-3-yl)ethyl]amino]-2- oxo-ethyl]-N,3,3-trimethyl-2- (2,2,2- trifluoroethylsulfonylamino) butanamide |
| 166 | 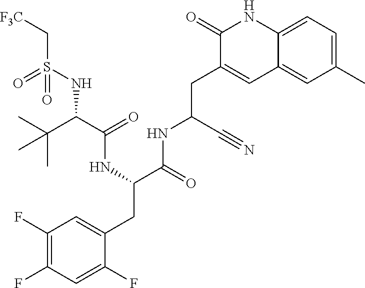 [SMILES: Cc1ccc2[nH]c(=O)c(CC(C#N)NC(=O)[C@H](Cc3cc(F)c(F)cc3F)NC(=O)[C@@H](NS(=O)(=O)CC(F)(F)F)C(C)(C)C)cc2c1] | (2S)-N-[(1S)-2-[[1-cyano-2-(6- methyl-2-oxo-1H-quinolin-3- yl)ethyl]amino]-2-oxo-1-[(2,4,5- trifluorophenyl)methyl]ethyl]- 3,3-dimethyl-2-(2,2,2- trifluoroethylsulfonylamino) butanamide |
| 167 | 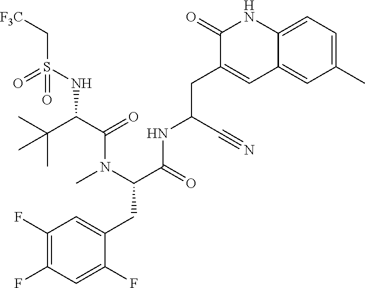 [SMILES: Cc1ccc2[nH]c(=O)c(CC(C#N)NC(=O)[C@H](Cc3cc(F)c(F)cc3F)N(C)C(=O)[C@@H](NS(=O)(=O)CC(F)(F)F)C(C)(C)C)cc2c1] | (2S)-N-[(1S)-2-[[1-cyano-2-(6- methyl-2-oxo-1H-quinolin-3- yl)ethyl]amino]-2-oxo-1-[(2,4,5- trifluorophenyl)methyl]ethyl]- N,3,3-trimethyl-2-(2,2,2- trifluoroethylsulfonylamino) butanamide |
| 168 | 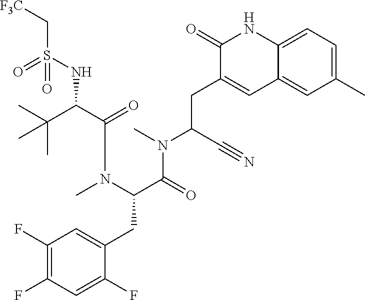 [SMILES: Cc1ccc2[nH]c(=O)c(CC(C#N)N(C)C(=O)[C@H](Cc3cc(F)c(F)cc3F)N(C)C(=O)[C@@H](NS(=O)(=O)CC(F)(F)F)C(C)(C)C)cc2c1] | (2S)-N-[(1S)-2-[[1-cyano-2-(6- methyl-2-oxo-1H-quinolin-3- yl)ethyl]-methyl-amino]-2-oxo-1- [(2,4,5- trifluorophenyl)methyl]ethyl]- N,3,3-trimethyl-2-(2,2,2- trifluoroethylsulfonylamino) butanamide |
| 169 | 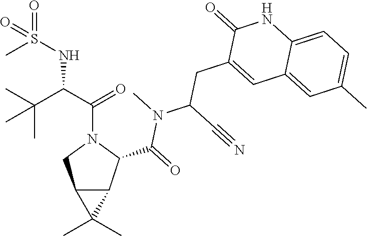 [SMILES: Cc1ccc2[nH]c(=O)c(CC(C#N)N(C)C(=O)[C@@H]3[C@H]4[C@H](CN3C(=O)[C@@H](NS(C)(=O)=O)C(C)(C)C)C4(C)C)cc2c1] | (1S,2S,5S)-N-[1-cyano-2-(6- methyl-2-oxo-1H-quinolin-3- yl)ethyl]-3-[(2S)-2- (methanesulfonamido)-3,3- dimethyl-butanoyl]-N,6,6- trimethyl-3- azabicyclo[3.1.0]hexane-2- carboxamide |
| 170 | 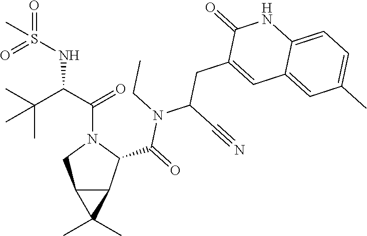 [SMILES: CCN(C(=O)[C@@H]1[C@@H]2[C@H](CN1C(=O)[C@@H](NS(C)(=O)=O)C(C)(C)C)C2(C)C)C(C#N)Cc1cc2cc(C)ccc2[nH]c1=O] | (1R,2S,5S)-N-[1-cyano-2-(6- methyl-2-oxo-1H-quinolin-3- yl)ethyl]-N-ethyl-3-[(2S)-2- (methanesulfonamido)-3,3- dimethyl-butanoyl]-6,6-dimethyl- 3-azabicyclo[3.1.0]hexane-2- carboxamide |
| 171 | 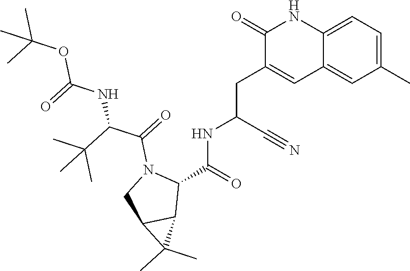 [SMILES: Cc1ccc2[nH]c(=O)c(CC(C#N)NC(=O)[C@@H]3[C@H]4[C@H](CN3C(=O)[C@@H](NC(=O)OC(C)(C)C)C(C)(C)C)C4(C)C)cc2c1] | tert-butyl N-[(1S)-1-[(1S,2S,5S)- 2-[[1-cyano-2-(6-methyl-2-oxo- 1H-quinolin-3- yl)ethyl]carbamoyl]-6,6- dimethyl-3- azabicyclo[3.1.0]hexane-3- carbonyl]-2,2-dimethyl- propyl]carbamate |
| 172 | 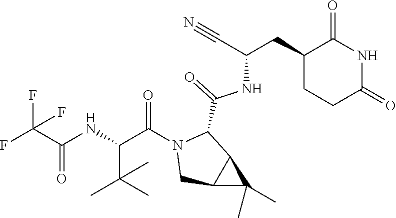 [SMILES: CC(C)(C)[C@H](NC(=O)C(F)(F)F)C(=O)N1C[C@H]2[C@@H]([C@H]1C(=O)N[C@H](C#N)C[C@@H]1CCC(=O)NC1=O)C2(C)C] | (1R,2S,5S)-N-[(1S)-1-cyano-2- [(3S)-2,6-dioxo-3- piperidyl]ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 173 | 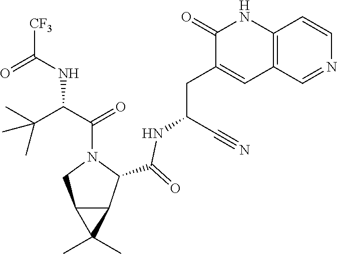 [SMILES: CC(C)(C)[C@H](NC(=O)C(F)(F)F)C(=O)N1C[C@H]2[C@@H]([C@H]1C(=O)N[C@@H](C#N)Cc1cc3cnccc3[nH]c1=O)C2(C)C] | (1R,2S,5S)-N-[(1R)-1-cyano-2- (2-oxo-1H-1,6-naphthyridin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 174 | 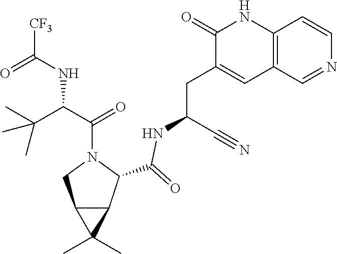 [SMILES: CC(C)(C)[C@H](NC(=O)C(F)(F)F)C(=O)N1C[C@H]2[C@@H]([C@H]1C(=O)N[C@H](C#N)Cc1cc3cnccc3[nH]c1=O)C2(C)C] | (1R,2S,5S)-N-[(1S)-1-cyano-2- (2-oxo-1H-1,6-naphthyridin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 175 | 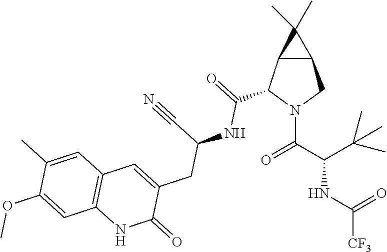 [SMILES: COc1cc2[nH]c(=O)c(C[C@@H](C#N)NC(=O)[C@@H]3[C@@H]4[C@H](CN3C(=O)[C@@H](NC(=O)C(F)(F)F)C(C)(C)C)C4(C)C)cc2cc1C] | (1R,2S,5S)-N-[(1S)-1-cyano-2- (7-methoxy-6-methyl-2-oxo-1H- quinolin-3-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 176 | 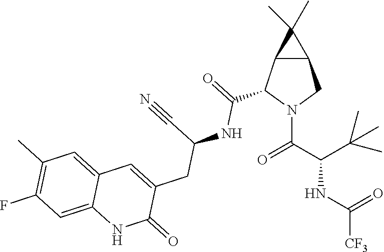 [SMILES: Cc1cc2cc(C[C@@H](C#N)NC(=O)[C@@H]3[C@@H]4[C@H](CN3C(=O)[C@@H](NC(=O)C(F)(F)F)C(C)(C)C)C4(C)C)c(=O)[nH]c2cc1F] | (1R,2S,5S)-N-[(1S)-1-cyano-2- (7-fluoro-6-methyl-2-oxo-1H- quinolin-3-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 177 | 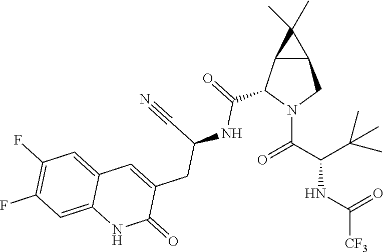 [SMILES: CC(C)(C)[C@H](NC(=O)C(F)(F)F)C(=O)N1C[C@H]2[C@@H]([C@H]1C(=O)N[C@H](C#N)Cc1cc3cc(F)c(F)cc3[nH]c1=O)C2(C)C] | (1R,2S,5S)-N-[(1S)-1-cyano-2- (6,7-difluoro-2-oxo-1H-quinolin- 3-yl)ethyl]-3-[(2S)-3,3-dimethyl- 2-[(2,2,2- trifluoroacetyl)amino]butanoyl]- 6,6-dimethyl-3- azabicyclo[3.1.0]hexane-2- carboxamide |
| 178 | 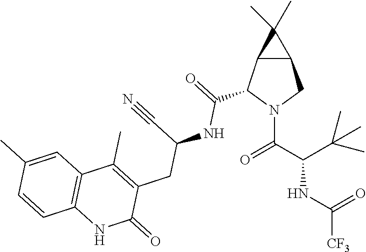 [SMILES: Cc1ccc2[nH]c(=O)c(C[C@@H](C#N)NC(=O)[C@@H]3[C@@H]4[C@H](CN3C(=O)[C@@H](NC(=O)C(F)(F)F)C(C)(C)C)C4(C)C)c(C)c2c1] | (1R,2S,5S)-N-[(1S)-1-cyano-2- (4,6-dimethyl-2-oxo-1H-quinolin- 3-yl)ethyl]-3-[(2S)-3,3-dimethyl- 2-[(2,2,2- trifluoroacetyl)amino]butanoyl]- 6,6-dimethyl-3- azabicyclo[3.1.0]hexane-2- carboxamide |
| 179 | 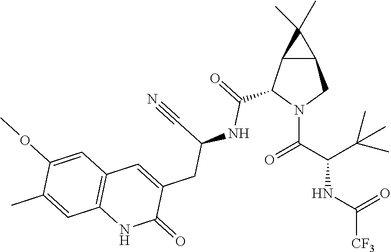 [SMILES: COc1cc2cc(C[C@@H](C#N)NC(=O)[C@@H]3[C@@H]4[C@H](CN3C(=O)[C@@H](NC(=O)C(F)(F)F)C(C)(C)C)C4(C)C)c(=O)[nH]c2cc1C] | (1R,2S,5S)-N-[(1S)-1-cyano-2- (6-methoxy-7-methyl-2-oxo-1H- quinolin-3-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 180 | 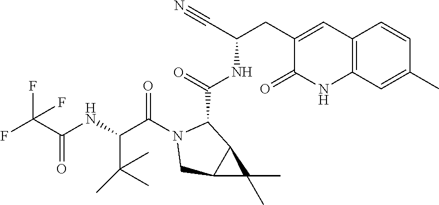 [SMILES: Cc1ccc2cc(C[C@@H](C#N)NC(=O)[C@@H]3[C@@H]4[C@H](CN3C(=O)[C@@H](NC(=O)C(F)(F)F)C(C)(C)C)C4(C)C)c(=O)[nH]c2c1] | (1R,2S,5S)-N-[(1S)-1-cyano-2- (7-methyl-2-oxo-1H-quinolin-3- yl)ethyl]-3-[(2S)-3,3-dimethyl-2- [(2,2,2- trifluoroacetyl)amino]butanoyl]- 6,6-dimethyl-3- azabicyclo[3.1.0]hexane-2- carboxamide |
| 181 | 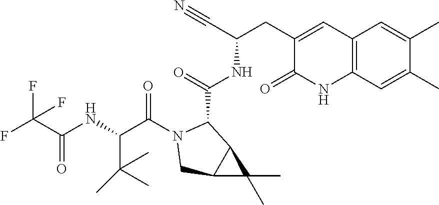 [SMILES: Cc1cc2cc(C[C@@H](C#N)NC(=O)[C@@H]3[C@@H]4[C@H](CN3C(=O)[C@@H](NC(=O)C(F)(F)F)C(C)(C)C)C4(C)C)c(=O)[nH]c2cc1C] | (1R,2S,5S)-N-[(1S)-1-cyano-2- (6,7-dimethyl-2-oxo-1H-quinolin- 3-yl)ethyl]-3-[(2S)-3,3-dimethyl- 2-[(2,2,2- trifluoroacetyl)amino]butanoyl]- 6,6-dimethyl-3- azabicyclo[3.1.0]hexane-2- carboxamide |
| 182 | 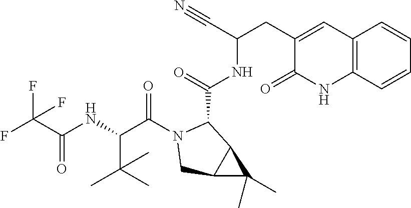 [SMILES: CC(C)(C)[C@H](NC(=O)C(F)(F)F)C(=O)N1C[C@H]2[C@@H]([C@H]1C(=O)NC(C#N)Cc1cc3ccccc3[nH]c1=O)C2(C)C] | (1R,2S,5S)-N-[1-cyano-2-(2-oxo- 1H-quinolin-3-yl)ethyl]-3-[(2S)- 3,3-dimethyl-2-[(2,2,2- trifluoroacetyl)amino]butanoyl]- 6,6-dimethyl-3- azabicyclo[3.1.0]hexane-2- carboxamide |
| 183 | 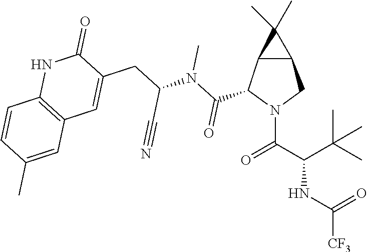 [SMILES: Cc1ccc2[nH]c(=O)c(C[C@@H](C#N)N(C)C(=O)[C@@H]3[C@@H]4[C@H](CN3C(=O)[C@@H](NC(=O)C(F)(F)F)C(C)(C)C)C4(C)C)cc2c1] | (1R,2S,5S)-N-[(1S)-1-cyano-2- (6-methyl-2-oxo-1H-quinolin-3- yl)ethyl]-3-[(2S)-3,3-dimethyl-2- [(2,2,2- trifluoroacetyl)amino]butanoyl]- N,6,6-trimethyl-3- azabicyclo[3.1.0]hexane-2- carboxamide |
| 184 | 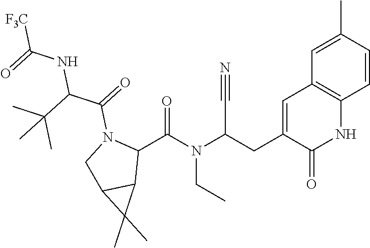 [SMILES: CCN(C(=O)C1C2C(CN1C(=O)C(NC(=O)C(F)(F)F)C(C)(C)C)C2(C)C)C(C#N)Cc1cc2cc(C)ccc2[nH]c1=O] | N-[1-cyano-2-(6-methyl-2-oxo- 1H-quinolin-3-yl)ethyl]-3-[3,3- dimethyl-2-[(2,2,2- trifluoroacetyl)amino]butanoyl]- N-ethyl-6,6-dimethyl-3- azabicyclo[3.1.0]hexane-2- carboxamide |
| 185 | 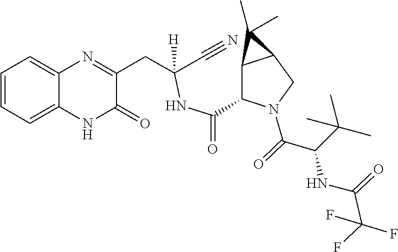 [SMILES: CC(C)(C)[C@H](NC(=O)C(F)(F)F)C(=O)N1C[C@H]2[C@@H]([C@H]1C(=O)N[C@H](C#N)Cc1nc3ccccc3[nH]c1=O)C2(C)C] | (1S,2S,5S)-N-[1-cyano-2-(3-oxo- 4H-quinoxalin-2-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 186 | 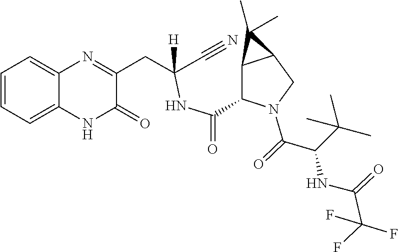 [SMILES: CC(C)(C)[C@H](NC(=O)C(F)(F)F)C(=O)N1C[C@H]2[C@@H]([C@H]1C(=O)N[C@@H](C#N)Cc1nc3ccccc3[nH]c1=O)C2(C)C] | (1S,2S,5S)-N-[1-cyano-2-(3-oxo- 4H-quinoxalin-2-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 187 | 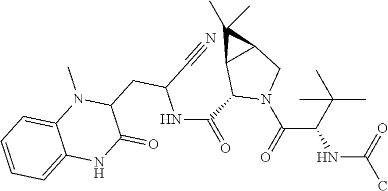 [SMILES: CC(=O)N[C@H](C(=O)N1C[C@H]2[C@@H]([C@H]1C(=O)NC(C#N)CC1C(=O)Nc3ccccc3N1C)C2(C)C)C(C)(C)C] | (1S,2S,5S)-N-[1-cyano-2-(1- methyl-3-oxo-2,4- dihydroquinoxalin-2-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 188 | 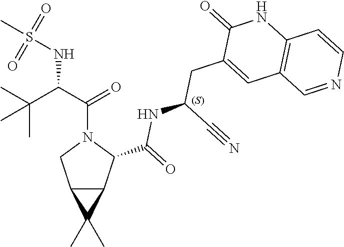 | (1S,2S,5S)-N-[(1S)-1-cyano-2-(2- oxo-1H-1,6-naphthyridin-3- yl)ethyl]-3-[(2S)-2- (methanesulfonamido)-3,3- dimethyl-butanoyl]-6,6-dimethyl-3- azabicyclo[3.1.0]hexane-2- carboxamide |
| 189 | 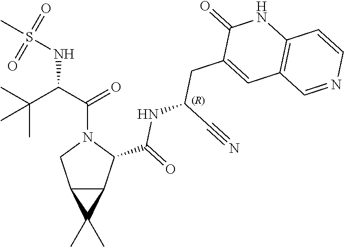 | (1S,2S,5S)-N-[(1R)-1-cyano-2- (2-oxo-1H-1,6-naphthyridin-3- yl)ethyl]-3-[(2S)-2- (methanesulfonamido)-3,3- dimethyl-butanoyl]-6,6-dimethyl- 3-azabicyclo[3.1.0]hexane-2- carboxamide |
| 190 | 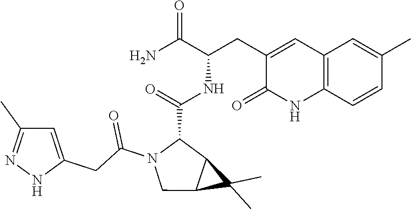 | (1R,2S,5S)-N-[(1S)-1-cyano-2- (6-methyl-2-oxo-1H-quinolin-3- yl)ethyl]-6,6-dimethyl-3-[2-(3- methyl-1H-pyrazol-5-yl)acetyl]- 3-azabicyclo[3.1.0]hexane-2- carboxamide |
| 191 | 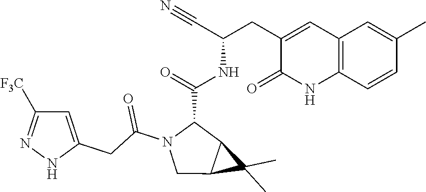 [SMILES: Cc1ccc2[nH]c(=O)c(C[C@@H](C#N)NC(=O)[C@@H]3[C@@H]4[C@H](CN3C(=O)Cc3cc(C(F)(F)F)n[nH]3)C4(C)C)cc2c1] | (1R,2S,5S)-N-[(1S)-1-cyano-2- (6-methyl-2-oxo-1H-quinolin-3- yl)ethyl]-6,6-dimethyl-3-[2-[3- (trifluoromethyl)-1H-pyrazol-5- yl]acetyl]-3- azabicyclo[3.1.0]hexane-2- carboxamide |
| 192 | 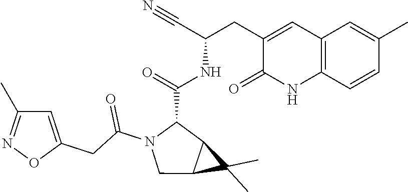 | (1R,2S,5S)-N-[(1S)-1-cyano-2- (6-methyl-2-oxo-1H-quinolin-3- yl)ethyl]-6,6-dimethyl-3-[2-(3- methylisoxazol-5-yl)acetyl]-3- azabicyclo[3.1.0]hexane-2- carboxamide |
| 193 | 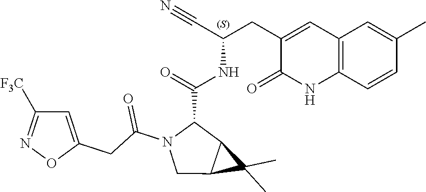 | (1R,2S,5S)-N-[(1S)-1-cyano-2- (6-methyl-2-oxo-1H-quinolin-3- yl)ethyl]-6,6-dimethyl-3-[2-[3- (trifluoromethyl)isoxazol-5- yl]acetyl]-3- azabicyclo[3.1.0]hexane-2- carboxamide |
| 194 | 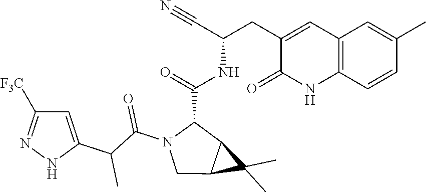 [SMILES: Cc1ccc2[nH]c(=O)c(C[C@@H](C#N)NC(=O)[C@@H]3[C@@H]4[C@H](CN3C(=O)C(C)c3cc(C(F)(F)F)n[nH]3)C4(C)C)cc2c1] | (1S,2S,5S)-N-[(1S)-1-cyano-2-(6- methyl-2-oxo-1H-quinolin-3- yl)ethyl]-6,6-dimethyl-3-[2-[3- (trifluoromethyl)-1H-pyrazol-5- yl]propanoyl]-3- azabicyclo[3.1.0]hexane-2- carboxamide |
| 195 | 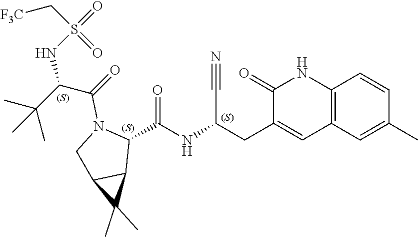 | (1S,2S,5S)-N-[(1S)-1-cyano-2-(6- methyl-2-oxo-1H-quinolin-3- yl)ethyl]-3-[(2S)-3,3-dimethyl-2- (2,2,2- trifluoroethylsulfonylamino) butanoyl]-6,6-dimethyl-3- azabicyclo[3.1.0]hexane-2- carboxamide |
| 196 | 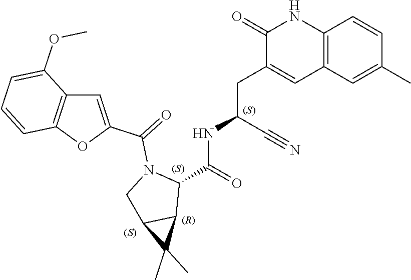 | (1R,2S,5S)-N-[(1S)-1-cyano-2- (6-methyl-2-oxo-1H-quinolin-3- yl)ethyl]-3-(4- methoxybenzofuran-2-carbonyl)- 6,6-dimethyl-3- azabicyclo[3.1.0]hexane-2- carboxamide |
| 197 | 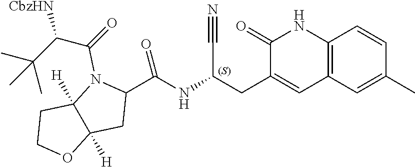 | benzyl N-[(1S)-1-[(3aR,6aR)-5- [[(1S)-1-cyano-2-(6-methyl-2- oxo-1H-quinolin-3- yl)ethyl]carbamoyl]- 2,3,3a,5,6,6a-hexahydrofuro[3,2- b]pyrrole-4-carbonyl]-2,2- dimethyl-propyl]carbamate |
| 198 | 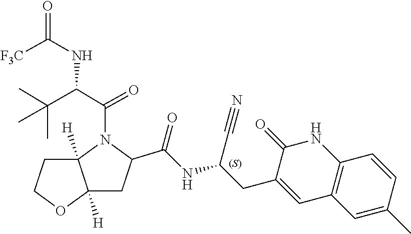 | (3aR,6aR)-N-[(1S)-1-cyano-2-(6- methyl-2-oxxo-1H-quinolin-3- yl)ethyl]-4-[(2S)-3,3-dimethyl-2- [(2,2,2- trifluoroacetyl)amino]butanoyl]- 2,3,3a,5,6,6a-hexahydrofuro[3,2- b]pyrrole-5-carboxamide |
| 199 | 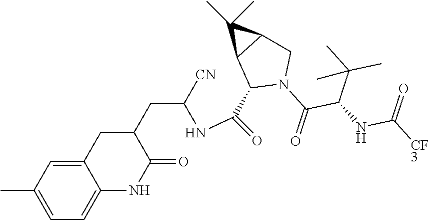 [SMILES: Cc1ccc2c(c1)CC(CC(C#N)NC(=O)[C@@H]1[C@@H]3[C@H](CN1C(=O)[C@@H](NC(=O)C(F)(F)F)C(C)(C)C)C3(C)C)C(=O)N2] | (1S,2S,5S)-N-[1-cyano-2-(6- methyl-2-oxo-3,4-dihydro-1H- quinolin-3-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 200 | 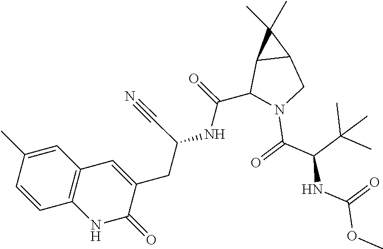 | methyl N-[(1R)-1-[(1S)-2-[[(1R)- 1-cyano-2-(6-methyl-2-oxo-1H- quinolin-3-yl)ethyl]carbamoyl]- 6,6-dimethyl-3- azabicyclo[3.1.0]hexane-3- carbonyl]-2,2-dimethyl- propyl]carbamate |
| 201 | 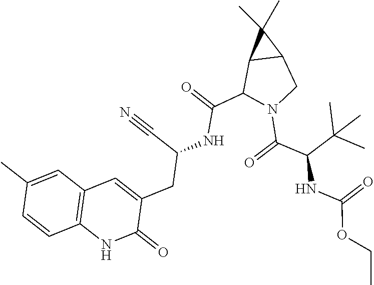 | ethyl N-[(1R)-1-[(1S)-2-[[(1R)-1- cyano-2-(6-methyl-2-oxo-1H- quinolin-3-yl)ethyl]carbamoyl]- 6,6-dimethyl-3- azabicyclo[3.1.0]hexane-3- carbonyl]-2,2-dimethyl- propyl]carbamate |
| 202 | 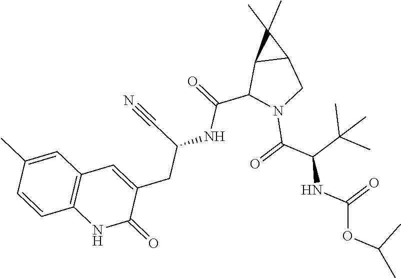 | isopropyl N-[(1R)-1-[(1S)-2- [[(1R)-1-cyano-2-(6-methyl-2- oxo-1H-quinolin-3- yl)ethyl]carbamoyl]-6,6- dimethyl-3- azabicyclo[3.1.0]hexane-3- carbonyl]-2,2-dimethyl- propyl]carbamate |
| 203 | 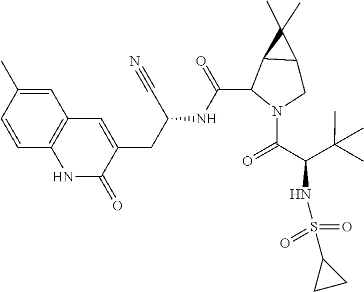 | (1S)-N-[(1R)-1-cyano-2-(6- methyl-2-oxo-1H-quinolin-3- yl)ethyl]-3-[(2R)-2- (cyclopropylsulfonylamino)-3,3- dimethyl-butanoyl]-6,6-dimethyl-3- azabicyclo[3.1.0]hexane-2- carboxamide |
| 204 | 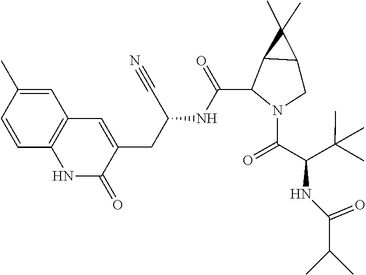 | (1S)-N-[(1R)-1-cyano-2-(6- methyl-2-oxo-1H-quinoin-3- yl)ethyl]-3-[(2R)-3,3-dimethyl-2- (2-methylpropanoylamino)butanoyl]- 6,6-dimethyl-3- azabicyclo[3.1.0]hexane-2- carboxamide |
| 205 | 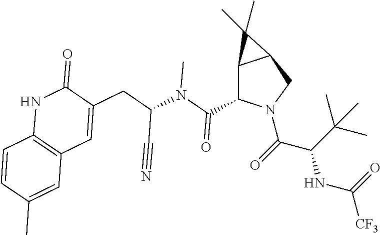 [SMILES: Cc1ccc2[nH]c(=O)c(C[C@@H](C#N)N(C)C(=O)[C@@H]3[C@@H]4[C@H](CN3C(=O)[C@@H](NC(=O)C(F)(F)F)C(C)(C)C)C4(C)C)cc2c1] | (1S,2S,5S)-N-[1-cyano-2-(6- methyl-2-oxo-1H-quinolin-3- yl)ethyl]-3-[(2S)-3,3-dimethyl-2- [(2,2,2-trifluoroacetyl)amino] butanoyl]-N,6,6-trimethyl-3- azabicyclo[3.1.0]hexane-2- carboxamide |
| 206 | 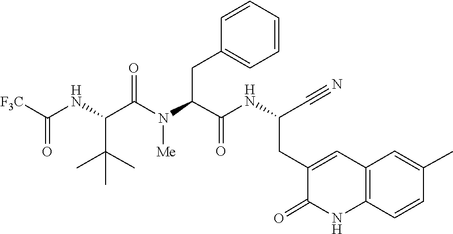 [SMILES: Cc1ccc2[nH]c(=O)c(C[C@@H](C#N)NC(=O)[C@H](Cc3ccccc3)N(C)C(=O)[C@@H](NC(=O)C(F)(F)F)C(C)(C)C)cc2c1] | (2S)-N-[(1S)-1-benzyl-2-[[(1S)-1- cyano-2-(6-methyl-2-oxo-1H- quinolin-3-yl)ethyl]amino]-2- oxo-ethyl]-N,3,3-trimethyl-2- [(2,2,2- trifluoroacetyl)amino]butanamide |
| 207 | 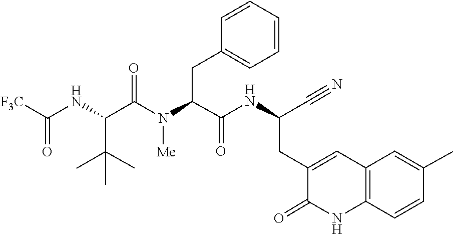 [SMILES: Cc1ccc2[nH]c(=O)c(C[C@H](C#N)NC(=O)[C@H](Cc3ccccc3)N(C)C(=O)[C@@H](NC(=O)C(F)(F)F)C(C)(C)C)cc2c1] | (2S)-N-[(1S)-1-benzyl-2-[[(1R)- 1-cyano-2-(6-methyl-2-oxo-1H- quinolin-3-yl)ethyl]amino]-2- oxo-ethyl]-N,3,3-trimethyl-2- [(2,2,2- trifluoroacetyl)amino]butanamide |
| 208 | 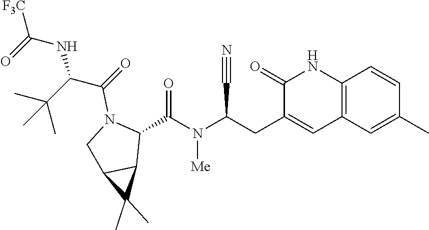 [SMILES: Cc1ccc2[nH]c(=O)c(C[C@H](C#N)N(C)C(=O)[C@@H]3[C@@H]4[C@H](CN3C(=O)[C@@H](NC(=O)C(F)(F)F)C(C)(C)C)C4(C)C)cc2c1] | (1R,2S,5S)-N-[(1R)-1-cyano-2- (6-methyl-2-oxo-1H-quinolin-3- yl)ethyl]-3-[(2S)-3,3-dimethyl-2- [(2,2,2-trifluoroacetyl)amino] butanoyl]-N,6,6-trimethyl-3- azabicyclo[3.1.0]hexane-2- carboxamide |
| 209 | 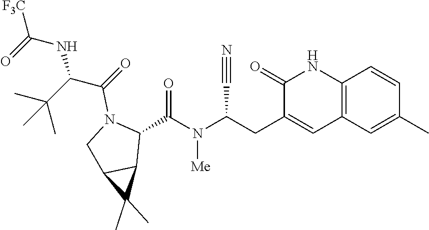 [SMILES: Cc1ccc2[nH]c(=O)c(C[C@@H](C#N)N(C)C(=O)[C@@H]3[C@@H]4[C@H](CN3C(=O)[C@@H](NC(=O)C(F)(F)F)C(C)(C)C)C4(C)C)cc2c1] | (1R,2S,5S)-N-[(1S)-1-cyano-2- (6-methyl-2-oxo-1H-quinolin-3- yl)ethyl]-3-[(2S)-3,3-dimethyl-2- [(2,2,2-trifluoroacetyl)amino]butanoyl]- N,6,6-trimethyl-3- azabicyclo[3.1.0]hexane-2- carboxamide |

or a pharmaceutically acceptable salt, stereoisomer, solvate, or tautomer thereof.

10. A pharmaceutical composition comprising the compound or pharmaceutically acceptable salt, solvate, hydrate, stereoisomer, or tautomer thereof of claim 1, and a pharmaceutically acceptable carrier.

11. The pharmaceutical composition of claim 10, further comprising an additional pharmaceutically active agent.

12. A method of inhibiting of coronavirus replication, comprising of administering to a subject a compound of claim 1.

13. A method of treating a disease or disorder associated with coronavirus infection, comprising of administering to a subject a compound of claim 1.

14. A method of treating coronavirus infection comprising of administering to a subject in need of a treatment a compound of claim 1.

15. The method of claim 14, wherein coronavirus infection is COVID-19.

16. The method of any one of claims 12-15, wherein the subject is a human.
